(19) United States
(12) Patent Application Publication
(10) Pub. No.: US 2025/0339569 A1
(43) Pub. Date: Nov. 6, 2025

(54) CARBONIC ANHYDRASE IX LIGANDS

(71) Applicant: 3B PHARMACEUTICALS GMBH, Berlin (DE)

(72) Inventors: Frank OSTERKAMP, Berlin (DE); Aileen HÖHNE, Berlin (DE); Matthias PASCHKE, Berlin (DE); Dirk ZBORALSKI, Berlin (DE); Eberhard SCHNEIDER, Michendorf OT Wildenbruch (DE); Christian HAASE, Berlin (DE); Jan UNGEWISS, Berlin (DE); Anne BREDENBECK, Berlin (DE); Christiane SMERLING, Berlin (DE); Ulrich REINEKE, Berlin (DE); Ina WILKENING, Berlin (DE)

(21) Appl. No.: 18/720,400

(22) PCT Filed: Dec. 19, 2022
(86) PCT No.: PCT/EP2022/086632

§ 371 Date: Jun. 14, 2024

(30) Foreign Application Priority Data
Dec. 17, 2021 (WO) PCT/EP2021/086592
Feb. 25, 2022 (WO) PCT/EP2022/054857
Mar. 18, 2022 (WO) PCT/EP2022/057170
Mar. 24, 2022 (WO) PCT/EP2022/057840
Jun. 24, 2022 (WO) PCT/EP2022/067424
Oct. 24, 2022 (WO) PCT/EP2022/079606
Nov. 11, 2022 (WO) PCT/EP2022/081674

(51) Int. Cl. A61K 51/08; A61P 35/00; C07K 7/64

(52) U.S. Cl. CPC A61K 51/088; A61P 35/00; C07K 7/64; A61K 2121/00; A61K 2123/00

(57) ABSTRACT

The present invention relates to a chemical compound; a peptide; a Carbonic Anhydrase IX (CAIX) binding compound; a CAIX binding peptide; a composition comprising the compound; a composition comprising the CAIX binding compound; a composition comprising the peptide; a composition comprising the CAIX peptide; the compound, CAIX binding compound, the peptide, the CAIX peptide and the compositions, respectively, for use in a method for the diagnosis of a disease; the compound, the CAIX binding compound and the compositions, respectively, for use in a method for the treatment of a disease; the compound, the CAIX binding compound, the peptide, the CAIX peptide and the compositions, respectively, for use in a method of diagnosis and treatment of a disease; the compound, the CAIX binding compound, the peptide, the CAIX peptide, and the compositions, respectively, for use in a method for delivering a radionuclide to a CAIX expressing tissue; a method for the diagnosis of a disease using the compound, the CAIX binding compound, the peptide, the CAIX peptide and the compositions, respectively; a method for the treatment of a disease using the compound, the CAIX binding compound, the peptide, the CAIX peptide and the compositions, respectively; a method for the diagnosis and treatment of a disease using the compound, the CAIX binding compound, the peptide, the CAIX peptide and the compositions, respectively; a method for the delivery of a radionuclide to a CAIX expressing tissue using the compound, the CAIX binding compound, the peptide, the CAIX peptide and the compositions, respectively.

### REFERENCE TO AN ELECTRONIC SEQUENCE LISTING

The contents of the electronic sequence listing (900307_401USPC_SeqListing.xml; Size: 12,217 bytes; and Date of Creation: Jan. 31, 2025) is herein incorporated by reference in its entirety.

### FIELD OF THE INVENTION

The present invention is related to a chemical compound; a peptide; a Carbonic Anhydrase IX (CAIX) binding compound; a Carbonic Anhydrase IX (CAIX) binding peptide; a composition comprising the compound; a composition comprising the Carbonic Anhydrase IX (CAIX) binding compound; a composition comprising the peptide; a composition comprising the Carbonic Anhydrase IX (CAIX) peptide; the compound, Carbonic Anhydrase IX (CAIX) binding compound, the peptide, the Carbonic Anhydrase IX (CAIX) peptide and the compositions, respectively, for use in a method for the diagnosis of a disease; the compound, the Carbonic Anhydrase IX (CAIX) binding compound and the compositions, respectively, for use in a method for the treatment of a disease; the compound, the Carbonic Anhydrase IX (CAIX) binding compound, the peptide, the Carbonic Anhydrase IX (CAIX) peptide and the compositions, respectively, for use in a method of diagnosis and treatment of a disease which is also referred to as “thera(g)nosis” or “thera(g)nostics”; the compound, the Carbonic Anhydrase IX (CAIX) binding compound, the peptide, the Carbonic Anhydrase IX (CAIX) peptide, and the compositions, respectively, for use in a method for delivering an effector e.g., a radionuclide to a Carbonic Anhydrase IX (CAIX) expressing tissue; a method for the diagnosis of a disease using the compound, the Carbonic Anhydrase IX (CAIX) binding compound, the peptide, the Carbonic Anhydrase IX (CAIX) peptide and the compositions, respectively; a method for the treatment of a disease using the compound, the Carbonic Anhydrase IX (CAIX) binding compound, the peptide, the Carbonic Anhydrase IX (CAIX) peptide and the compositions, respectively; a method for the diagnosis and treatment of a disease which is also referred to as “thera(g)nosis” or “thera(g)nostics”, using the compound, the Carbonic Anhydrase IX (CAIX) binding compound, the peptide, the Carbonic Anhydrase IX (CAIX) peptide and the compositions, respectively; a method for the delivery of an effector e.g., a radionuclide to a Carbonic Anhydrase IX (CAIX) expressing tissue using the compound, the Carbonic Anhydrase IX (CAIX) binding compound, the peptide, the Carbonic Anhydrase IX (CAIX) peptide and the compositions, respectively.

### BACKGROUND

Despite the increasing availability of therapeutic options, cancer is still the second leading cause of death globally. Rapidly proliferating cells have a high demand for nutrients and oxygen. This often leads to hypoxic conditions in cancer tissue since its vasculature is not able to supply them sufficiently (Brown et al., Nat Rev Cancer, 2004, 4, 437-447). Hypoxia is a feature of most solid tumors, with variable incidence and severity within a given patient population (Bhandari et al., Nat Genet, 2019, 51, 308-318).

The reduction of available oxygen triggers increased expression of hypoxia-inducible factor 1α (HIF-1α) (Cassavaugh et al., J Cell Biochem, 2011, 112, 735-744; Zhong et al., Cancer Res, 1999, 59, 5830-5835). This transcription factor induces several mechanisms to confer continued growth and drug resistance (Comerford et al., Cancer Res, 2002, 62, 3387-3394; Jing et al., Mol Cancer, 2019, 18, 157). To produce sufficient energy, cancer cells undergo a metabolic shift, triggered by HIF-1α, towards an increased glycolytic rate. This change leads to a steady supply of energy but also increases the production of acidic metabolites.

A side effect of the tumor's compensatory mechanisms to allow continued growth with an undersupply of oxygen is reduced drug and radiotherapy sensitivity. These additional effects make hypoxia a prognostic for poor patient outcomes (Walsh et al., Antioxid Redox Signal, 2014, 21, 1516-1554; van Kuijk et al., Front Oncol, 2016, 6, 69). To overcome this, specific targeting of the hypoxic cancer cells and their microenvironment is a promising approach for future therapies (Paolicchi et al., Oncotarget, 2016, 7, 13464-13478).

Human Carbonic Anhydrase IX (CAIX) was originally identified as membrane-bound protein in HeLa cells and other human carcinomas and was named “MN protein” (Zavada et al., Int J Cancer, 1993, 54, 268-274). Shortly thereafter, its extracellular carbonic anhydrase domain was identified, resulting in the renaming to Carbonic Anhydrase IX (Pastorek et al., Oncogene, 1994, 9, 2877-2888). CAIX is a major effector of the HIF-1α-mediated transcriptional response to tumor hypoxia and its critical role in tumor progression is well-recognized. In recent years, CAIX has gained notoriety as a surrogate marker of tumor hypoxia which is widely spread in solid tumors. Due to its low expression in non-cancerous tissues, it has become a target of interest for both diagnostic and therapeutic molecules (Lau et al., Theranostics, 2017, 7, 4322-4339). CAIX plays a significant role in the cellular pH homeostasis by catalyzing the interconversion between carbon dioxide and water and the dissociated ions of carbonic acid.

The human CAIX protein is encoded by the CA9 gene placed on the 9p12-13 chromosomal locus and composed of 11 exons coding for distinct structural domains (Opavský et al., Genomics, 1996, 33, 480-487). The enzyme consists of 4 domains, an N-terminal proteoglycan-like domain, a catalytic domain including the zinc ion, a transmembrane segment, and an intracytoplasmic portion. CAIX is a 459 amino acid 58/54 kDa metalloenzyme. It assembles as a dimer which is stabilized by the formation of an intermolecular disulfide bond between the same cysteine residue located on two carbonic anhydrase catalytic domains (Whittington et al., Proc Natl Acad Sci USA, 2001, 98, 9545-9550). The active site is located in a large conical cavity which spans from the surface to the center of the protein. The zinc ion is located at the bottom of this cavity (Alterio et al., Proc Natl Acad Sci USA, 2009, 106, 16233-16238). Additional post-translational modifications of the extracellular domain of CAIX include N-glycosylation by high mannose sugar chain in the catalytic domain and O-glycosylation by heparan or chondroitin sulfate glycosaminoglycan chains in the N-terminal proteoglycan-like region.

CAIX normal expression is limited to the epithelium of the stomach, bile duct, gallbladder duct, pancreatic duct, rapidly-proliferating normal cells of the small intestine, and, to a lower extent, to the CNS where it can be found mainly in the ventricular-lining cells and the choroid plexus (Zamanova et al., Expert Opin Ther Pat, 2019, 29, 509-533). On the other hand, CAIX expression is upregulated in most types of solid tumors including but not limited to breast (Storci et al., J Pathol, 2008, 214, 25-37), kidney (Luong-Player et al., Am J Clin Pathol, 2014, 141, 219-225), colon (Korkeila et al., Br J Cancer, 2009, 100, 874-880), ovarian (Choschzick et al., Virchows Arch, 2011, 459, 193-200), head-and-neck (Kappler et al., Strahlenther Onkol, 2008, 184, 393-399), pancreatic (Juhasz et al., Aliment Pharmacol Ther, 2003, 18, 837-846) and lung cancer (Ilie et al., Br J Cancer, 2010, 102, 1627-1635). In clear cell renal cell carcinomas, CAIX expression is unique compared to other cancers as it is commonly uncoupled from the hypoxia-induced signaling cascade (Shuin et al., Cancer Res, 1994, 54, 2852-2855).

TABLE 1
Distribution of CAIX in normal and pathologic tissues
(modified from Zamanova et al. (Zamanova et al.,
Expert Opin Ther Pat, 2019, 29, 509-533))

| Status | Biodistribution | Method of assessment |
|---|---|---|
| Normal | Gastrointestinal tract: | Immunostaining |
| | epithelium of stomach | |
| | bile duct | |
| | gallbladder duct | |
| | pancreatic duct | |
| | rapidly-proliferating normal | |
| | cells of the small intestine | |
| | CNS: | |
| | ventricular-lining cells | |
| | choroid plexus | |
| | Tumors: | Imaging/detection via |
| | renal cell carcinoma, colon, | CAIX antibodies and |
| | lung, breast, ovarian, head, | CAIX inhibitors, |
| | and neck, pancreatic cancer, | Immunostaining, |
| | transitional cell carcinoma | Western Blot |
| | of the urinary tract | |
| Diseased | Blood: | ELISA |
| | renal cell carcinoma | |
| | non-small cell lung cancer | |
| | Urine: | Western Blot |
| | transitional cell carcinoma | |
| | of the urinary tract | |

Carbonic anhydrases are a family of zinc metalloenzymes that catalyze the reversible hydration/dehydration of carbon dioxide/bicarbonate ion. This reaction forms the basis for the regulation of acid-base balance in organisms. During evolution, at least 15 carbonic anhydrase (CA) isoenzymes have emerged in humans which are major players in many physiological processes, including renal and male reproductive tract acidification, bone resorption, respiration, gluconeogenesis, signal transduction, and formation of gastric acid (Breton, JOP, 2001, 2, 159-164; Sly et al., Annu Rev Biochem, 1995, 64, 375-401). Three of those 15 human CA isoforms do not possess a catalytic activity because they do not contain the zinc ion and thus are called carbonic anhydrase-related proteins (CARPs). The CA isoforms possess variable levels of catalytic activity, different cellular localization, patterns of multimerization, domain organization, and attachment to membranes.

TABLE 2
Distribution of human CA isoforms (modified from Aggarwal et al.
(Aggarwal et al., J Enzyme Inhib Med Chem, 2013, 28, 267-277))

| Iso- | | |
|---|---|---|
| form | Sub-cellular | Tissue/Organ |
| CAI | Cytosol | Red blood cells, Gastrointestinal tract |
| CAII | Cytosol | Red blood cells, Gastrointestinal tract, |
| | | eyes, Osteoclasts, kidneys, lungs, |
| | | testes, brain |
| CAIII | Cytosol | Skeletal muscles, adipocytes |
| CAIV | Membrane-bound | Kidneys, lungs, pancreas, brain, |
| | | capillaries, colon, heart muscles |
| CAVA | Mitochondria | Liver |
| CAVB | Mitochondria | Heart and skeletal muscles, pancreas, |
| | | kidneys, Gastrointestinal tract, |
| | | spinal cord |
| CAVI | Secretory | Salivary and mammary glands |
| | (milk/salvia) | |
| CAVII | Cytosol | central nervous system |
| CAVIII* | Cytosol | central nervous system |
| CAIX | Transmembrane | Tumors, Gastrointestinal mucosa |
| CAPX* | Cytosol | central nervous system |
| CAXI* | Cytosol | central nervous system |
| CAXII | Transmembrane | Renal, intestinal, reproductive epithelia, |
| | | eye, tumors |
| CAXIII | Cytosol | Kidneys, brain, lungs, gut, |
| | | reproductive tract |
| CAXIV | Transmembrane | Kidneys, brain, liver |

*Carbonic anhydrase-related protein

The family of carbonic anhydrases has been divided into 5 classes: a (found in mammals, prokaryotes, algae, and fungi), R (found mainly in plants and some prokaryotes), 7 (present only in some forms of bacteria), and two other sub-classes: 6 and ((similar to class p, found in diatoms) (Aggarwal et al., Bioorg Med Chem, 2013, 21, 1526-1533). The three main classes (α, β, and γ) of CA are structurally dissimilar and are thought to have evolved independently, possibly as a result of convergent evolution. Based on cellular and subcellular location, the class of a carbonic anhydrases is classified into four different groups: cytosolic (CA I, II, III, VII, XIII); mitochondrial (CA VA, VB); secretory (CAVI), and membrane-associated (CA IV, IX, XII, XIV). The α-carbonic anhydrases are very closely related with an average of >39% of primary sequence identity amongst them (Pinard et al., Biomed Res Int, 2015, 2015, 453543). A majority of the sequence identity translates to residues located in the active site. This needs to be taken into account when developing a drug for a specific carbonic anhydrase target.

TABLE 3
Primary sequence identity in percent (bottom left) and the number of conserved
residues (top right) (CAIX is shown in bold, information adapted from Pinard
et al. (Pinard et al., Biomed Res Int, 2015, 2015, 453543))

| | I | II | III | IV | VA | VB | VI | VII | IX | XII | XIII | XIV |
|---|---|---|---|---|---|---|---|---|---|---|---|---|
| I | — | 158 | 141 | 78 | 126 | 128 | 82 | 132 | 83 | 91 | 154 | 85 |
| II | 61% | — | 152 | 88 | 133 | 138 | 90 | 147 | 85 | 89 | 157 | 96 |
| III | 54% | 59% | — | 82 | 120 | 117 | 87 | 130 | 80 | 86 | 151 | 90 |
| IV | 30% | 34% | 31% | — | 89 | 93 | 97 | 90 | 84 | 91 | 84 | 62 |
| VA | 48% | 51% | 45% | 24% | — | 184 | 93 | 131 | 83 | 84 | 124 | 88 |
| VB | 47% | 52% | 44% | 23% | 59% | — | 82 | 134 | 89 | 79 | 131 | 88 |
| VI | 32% | 34% | 32% | 27% | 28% | 24% | — | 93 | 107 | 104 | 90 | 106 |
| VII | 51% | 56% | 50% | 32% | 49% | 49% | 35% | — | 95 | 103 | 139 | 97 |
| IX | 33% | 34% | 31% | 27% | 32% | 33% | 39% | 37% | — | 101 | 90 | 113 |
| XII | 36% | 34% | 32% | 28% | 32% | 30% | 38% | 38% | 39% | — | 91 | 123 |
| XIII | 59% | 60% | 58% | 28% | 46% | 48% | 33% | 53% | 35% | 35% | — | 98 |
| XIV | 34% | 36% | 34% | 29% | 32% | 29% | 36% | 36% | 44% | 46% | 37% | — |

CAII has the widest distribution in the body, being expressed in the cytosol of cells from virtually every tissue or organ. The impact of this CA isozyme in the human body is best exemplified by CAII deficiency syndrome, a human autosomal recessive disorder characterized by osteopetrosis, renal tubular acidosis, and cerebral calcification (Shah et al., Hum Mutat, 2004, 24, 272).

CAIV is membrane-bound via a glycosylphosphatidylinositol anchor. The isozyme is expressed in bone marrow, gastrointestinal tract, liver, and gallbladder, whereas low expression is observed in the pancreas, kidney, brain, adipose, and soft tissues. CAIV mRNA expression in cancer is much lower than for other CAs (e.g. CAXIV) but can be observed in gliomas, renal cell carcinomas, thyroid cancers, and melanomas (Mboge et al., Metabolites, 2018, 8).

CAXII, similar to CAIX, is another membrane-bound isozyme, which was found to be expressed in various types of cancer and can be induced under hypoxic conditions (Wykoff et al., Cancer Res, 2000, 60, 7075-7083). It contains the N-terminal extracellular catalytic domain, an α-helical transmembrane region, and a small intracytoplasmic C-terminal domain, as does CAIX, but it does not have a proteoglycan domain (Whittington et al., Proc Natl Acad Sci USA, 2001, 98, 9545-9550). Similarly, with CAIX, it forms a dimer with the two active sites oriented towards the extracellular milieu. The catalytic domain contains two asparagine residues that can be glycosylated (Asn-52 and Asn-136). CAXII is upregulated in several cancers, including breast, renal, colorectal, non-small cell lung cancer, etc. (Waheed et al., Gene, 2017, 623, 33-40). Both CAIX and CAXII are overexpressed under hypoxic conditions. The expression patterns of CAIX and CAXII are different and they overlap only marginally.

Carbonic anhydrase XIV is another membrane-bound isozyme of CA with an extracellular catalytic domain, a single transmembrane helix, and a short intracellular polypeptide segment. It shares a more than 40% sequence identity with CAIX. CAXIV mRNA shows strong expression in the healthy brain, muscles, seminal vesicles, and retina and is upregulated in many cancers, being most often observed in melanomas, gliomas, liver, and uterine cancers (Mboge et al., Metabolites, 2018, 8).

Additionally, there are three known human catalytically inactive isoforms of α-carbonic anhydrases (VIII, X, and XI) which are known as carbonic anhydrase-related proteins (CARPs). These cytosolic isoforms lack CA activity apparently because of substitutions to one or more of the three functionally important histidine residues to coordinate the zinc atom (Tashian et al., EXS, 2000, 105-120). Most of these CARPs are predominantly expressed in the central nervous system.

Two main compound classes have been explored for targeting CAIX: antibodies and small molecules. Antibodies and their derivatives have been investigated for inhibiting expression or function of CAIX, stimulating immune response or delivery of cytotoxic payloads. CAIX-modulating small molecules with mainly inhibitory but also activating properties have been described. So far, few peptide-based approaches have been disclosed.

Typically, the compounds of the prior art targeting CAIX suffer from at least one of the following shortcomings rendering them unsuitable for use in the diagnosis and treatment, respectively, of a subject such as a human being: lack of Carbonic Anhydrase selectivity and lack of CAIX sensitivity in particular, low tumor-to-background ratio, increased background noise and low stability.

International patent application WO 2012/016713 disclosed CAIX-targeted polypeptides comprising the amino acid sequence YNTNHVPLSPKY (SEQ ID NO: 1) or a sequence variant thereof. The example part of WO 2012/016713 shows the use of ¹²⁵I-labeled CAIX-targeting peptides for visualizing their tumor-targeting abilities by means of whole-body planar imaging. The ¹³¹I-labeled version of the CAIX-targeting peptides was used for assessing their organ distribution. Those organ distribution experiments revealed low tumor-to-blood ratios and increased background noise, which is not favorable for imaging applications (Rana et al., PLoS One, 2012, 7, e38279). Another study by the same group aimed for the identification and the development of further novel peptides with affinity for regions of the extracellular domain of CAIX with no homology to other members of the CA family. A linear dodecapeptide NMPKDVTTRMSS (SEQ ID NO: 2) was identified by phage display and shown to selectively bind to the proteoglycan domain of CAIX but displayed an unfavorable biodistribution (Rana et al., Mol Imaging, 2013, 12), hampering its use as diagnostic or therapeutic agent. The reason for the poor performance of these peptides might be related to, but not limited by their low stability.

WO 2020/084305 and WO 2020/148526 disclosed polypeptides binding to CAIX with high affinity, which are covalently bound to molecular scaffolds such that two or more peptide loops are subtended between attachment points to the scaffold. The example part of WO 2020/084305 and WO 2020/148526 revealed very limited data on the in vitro activity of selected peptides in a CAIX competition binding assay and a CAIX enzyme inhibition assay. No data on CA isotype selectivity, stability or in vivo performance of the described peptides was disclosed. Demonstrating the ability to conjugate an effector to the CAIX-targeting peptide without significant loss of binding affinity to CAIX is limited to a single example, namely conjugation of the cytostatic agent DM-1 (mertansine) to 61-01-02-N003.

US2021154334A1 disclosed dual-targeted carbonic anhydrase IX complex comprising a binding peptide with the amino acid sequence NHYPLSP (SEQ ID NO: 3), or a fragment or derivative thereof, a sulfonamide derivative coupled with the binding peptide; and a metal chelating agent coupled with the binding peptide and the sulfonamide derivative. ¹¹¹In-DOTA-AAZ-CA9tp displayed high intestinal uptake at the early time points after intravenous injection, which was clearing over time, leading to gradual improvement of the initially low tumor/large intestine uptake ratio. No data on the selectivity of the compound for CAIX over other carbonic anhydrases were shown.

The above overview of the prior art attempting to provide a compound which can be used in the diagnosis and/or therapy of CAIX-expressing tumors, whereby such diagnosis and therapy typically make use of a radiolabeled version of such compound, illustrates the difficulties in designing this kind of compounds.

A preferred compound for the diagnosis and/or therapy of CAIX-expressing tumors may show at least one of the following properties, preferably two or more thereof, namely high binding affinity, high biological stability, high target selectivity as well as appropriate in vivo targeting and pharmacokinetic properties. A high binding affinity may facilitate uptake and retention of the compound in target-expressing tissues, so that it can exercises its biological effect in the tissue of interest (e.g., tumor). High biological stability is advantageous for availability of intact compound for a sufficient time to allow delivery to the tissue of interest. Compared to the intact compound, metabolites are likely to lose target affinity as well as to display a different in vivo distribution, potentially leading to loss of efficacy and occurrence of unwanted side effects. High target selectivity is desired in order to avoid off-target activity, which may contribute to side effects. Appropriate in vivo targeting and pharmacokinetic properties is helpful in ensuring appropriate delivery to and exposure of the tissue of interest with the compound, a prerequisite for its diagnostic and/or therapeutic efficacy.

### DETAILED DESCRIPTION OF THE INVENTION

The problem underlying the present invention is the provision of a compound which is suitable as a diagnostic agent and/or a therapeutic agent, particularly if conjugated to a diagnostically and/or therapeutically active radionuclide.

A further problem underlying the present invention is the provision of a compound which is suitable as a diagnostic agent and/or a therapeutic agent, particularly if it comprises a diagnostically and/or therapeutically active radionuclide, said compound having a pEC₅₀ of equal to or greater than 6.0 and/or a pIC₅₀ of equal to or greater than 6.0 for Carbonic Anhydrase IX (CAIX).

A further problem underlying the present invention is the provision of a compound which is suitable as a diagnostic agent and/or a therapeutic agent, particularly if it comprises a diagnostically and/or therapeutically active radionuclide, in the diagnosis and/or therapy of a disease where the diseased cells and/or diseased tissues express Carbonic Anhydrase IX (CAIX). A still further problem underlying the instant invention is the provision of a compound which is suitable for delivering a diagnostically and/or therapeutically effective radionuclide to a diseased cell and/or diseased tissue, respectively, and more particularly a CAIX-expressing diseased cell and/or diseased tissue, preferably the diseased tissue comprises or cancer or tumor cells.

Also, a problem underlying the present invention is the provision of a method for the diagnosis of a disease, of a method for the treatment and/or prevention of a disease, and a method for the combined diagnosis and treatment of a disease; preferably such disease is a disease involving CAIX-expressing cells and/or tissues, more particularly a CAIX-expressing diseased cell and/or diseased tissue, preferably the diseased tissue comprises or contains cancer or tumor cells.

A still further problem underlying the present invention is the provision of a method for the identification of a subject, wherein the subject is likely to respond or likely not to respond to a treatment of a disease, a method for the selection of a subject from a group of subjects, wherein the subject is likely to respond or likely not to respond to a treatment of a disease; preferably, the disease is cancer, more preferably the disease is a solid tumor.

Also, a problem underlying the present invention is the provision of a pharmaceutical composition containing a compound having the characteristics as outlined above. Furthermore, a problem underlying the present invention is the provision of a kit which is suitable for use in any of the above methods.

These and other problems are solved by the subject matter of the attached independent claims; preferred embodiments may be taken from the attached dependent claims.

The problem underlying the present invention is also solved in a first aspect, which is also a first embodiment of the first aspect, by a compound comprising a peptide selected from the group consisting of
a cyclic peptide of formula (1a)

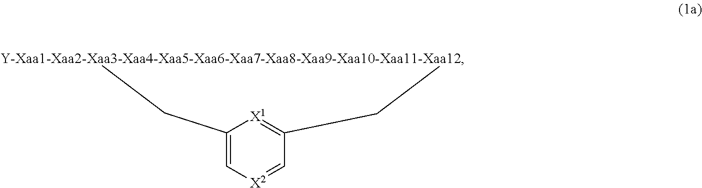

wherein, in formula (1a), the peptide sequence is drawn from left to right in N-terminal to C-terminal direction, and
   Y
      (i) is an N-terminal modification group A selected from the group consisting of R^{0a}—SO2-, R^{0a}—CO—, R^{0a}—NH—CO—, wherein
      R^{0a} is selected from the group consisting of (C₁-C₁₀)alkyl, (C₅-C₁₀)aryl, and (C₁-C₅)alkyl-(C₅-C₁₀)aryl,
      or
      (ii) comprises an effector E1, such as a chelator, wherein the effector E1 is covalently bound to Xaa1 if Xaa1 is present, or to Xaa2 if Xaa1 is absent and Xaa2 is present, or to Xaa3 if both Xaa1 and Xaa2 are absent,
      or
      (iii) is Z1, wherein Z1 comprises a linker moiety L1 and an effector E1, such as a chelator, wherein the linker moiety L1 covalently links the effector E1 to Xaa1 if Xaa1 is present, or to Xaa2 if Xaa1 is absent and Xaa2 is present, or to Xaa3 if both Xaa1 and Xaa2 are absent;
   Xaa1 is either present or absent, and if present is a residue of an aliphatic or polar L-amino acid;
   Xaa2 is either present or absent, wherein
      if Xaa2 is absent, Xaa1 is also absent and,
      if Xaa2 is present,
   (i) Xaa2 is a residue of an L-α-amino acid which is optionally N-methylated at the α-nitrogen atom,
   or,
   (ii) Xaa2 is a residue of an L-α-amino acid comprising, in addition to an amino group and a carboxy group attached to an α-C atom, a functional group FG1 forming a covalent linkage B1 with a functional group FG2 of Xaa11, wherein Xaa11 is a residue of an L-α-amino acid comprising, in addition to an amino group and a carboxy group attached to an α-C atom, the functional group FG2, wherein a bicyclic peptide of formula (1b) is formed:

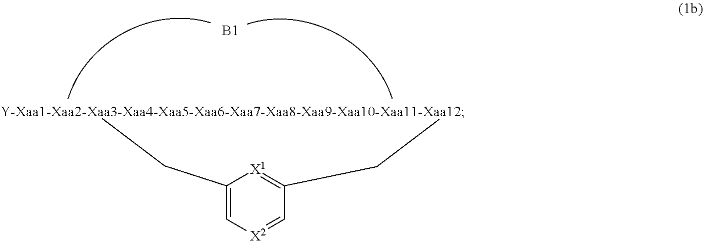

Xaa3 is a residue of an α-amino acid of formula (X)

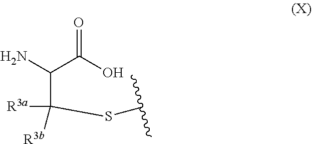

wherein
   R^{3a} and R^{3b} are each and independently selected from the group consisting of H and CH₃; and
   Xaa3 is preferably is a residue of an L-α-amino acid such as Cys;
Xaa4 is a residue of an L-α-amino acid which is optionally N-methylated at the α-nitrogen atom;
Xaa5 is a residue of an amino acid which is optionally bound to Z3, wherein Xaa5 is a residue of an amino acid selected from the group consisting of N—(C₁-C₆)alkyl glycine, Gly, a D-α-amino acid, and an α,α-dialkylamino acid,
   wherein if Xaa5 comprises Z3,
   (i) Z3 is an effector E3, such as a chelator, Xaa5 is preferably a residue of an amino acid selected from the group consisting 4-aminobutyl-glycine [Nlys], D-lys, (R)-ornithine [D-orn], (R)-2,4-diaminobutyric acid [D-dab], and (R)-2,3-diaminopropionic acid [D-dap], and the effector is attached to an N atom different from the α-nitrogen atom of any one of Nlys, D-lys, D-orn, D-dab, and D-dap, or
   (ii) Z3 comprises an effector E3, such as a chelator, and a linker moiety L3, Xaa5 is preferably a residue of an amino acid selected from the group consisting of Nlys, D-lys, D-orn, D-dab, and D-dap, and the linker moiety L3 is attached to an N atom different from the α-nitrogen atom of any one of Nlys, D-lys, D-orn, D-dab, and D-dap;
Xaa6 (i) is a residue of an amino acid which is selected from the group consisting of a polar L-α-amino acid, an aromatic L-α-amino acid, an aliphatic α-amino acid, an S-alkylated cysteine, an oxidized form of an S-alkylated cysteine, and a residue of an amino acid according to formula (3),

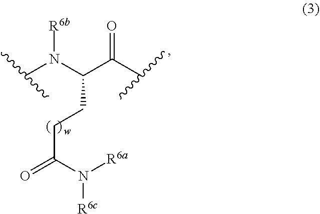

wherein
   R^{6a} is selected from the group consisting of H a moiety comprising a —(C₅-C₁₀)aryl, (C₁-C₈)alkyl, and (C₁-C₅)alkyl-(C₅-C₁₀)aryl,
   R^{6b} is selected from the group consisting of H and methyl,
   R^{6c} is H or (C₁-C₆)alkyl, and
   w is 0 or 1,
   or
   (ii) is a residue of an L-α-amino acid comprising, in addition to an amino group and a carboxy group attached to an α-C atom, a functional group FG3 forming a covalent linkage B2 with a functional group FG4 of Xaa11, wherein Xaa11 is a residue of an α-amino acid comprising, in addition to an amino group and a carboxy group attached to an α-C atom, the functional group FG4, wherein a bicyclic peptide of formula (1c) is formed:

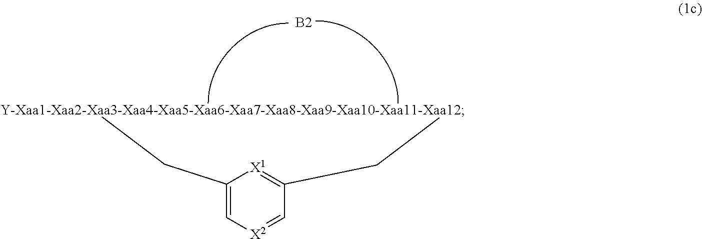

Xaa7 is a residue of an amino acid which is selected from the group consisting of an aromatic amino acid, such as a heteroaromatic L-α-amino acid, and a substituted aromatic amino acid, such as a substituted heteroaromatic L-α amino acid;
Xaa8 is a residue of an amino acid which is selected from the group consisting of an L-α-amino acid and a cyclic α,α-dialkyl amino acid;
Xaa9 is a residue of an amino acid which is selected from the group consisting of Gly and an L-α-amino acid;
Xaa10 is a residue of a heteroaromatic L-α-amino acid;
Xaa11 (i) is a residue of an amino acid which is selected from the group consisting of Gly and an L-α-amino acid, wherein the L-α-amino acid is optionally bound to Z4, wherein Z4 comprises an effector E4, such as a chelator, and a linker moiety L4, or
(ii) is a residue of an L-α-amino acid comprising, in addition to an amino group and a carboxy group attached to an α-C atom, the functional group FG2 forming the covalent linkage B1 with the functional group FG1 of Xaa2; or
(iii) is a residue of an α-amino acid comprising, in addition to an amino group and a carboxy group attached to an α-C atom, the functional group FG4 forming the covalent linkage B2 with the functional group FG3 of Xaa6;
   Xaa12 is a residue of an α-amino thiol of formula (XII):

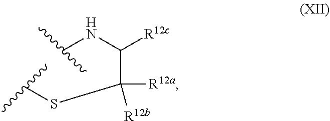

preferably of formula (XIIa):

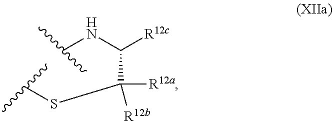

wherein
the NH of of each of formulae (XII) and (XIIa) is bound to Xaa11;
R^{12a} and R^{12b} are each and independently selected from the group consisting of H and CH₃; and
R^{12c} is selected from the group consisting of —CO—OH, CO—NH2, —CO—Z6 and —CH₂—Z6, wherein Z6 comprises a linker moiety L6 and an effector E6, such as a chelator; and
X¹ and X² are each and independently selected from the group consisting of C—H and N.

The problem underlying the present invention is solved in a second aspect, which is also a first embodiment of the second aspect, by a peptide selected from the group consisting of:
a cyclic peptide of formula (1a):

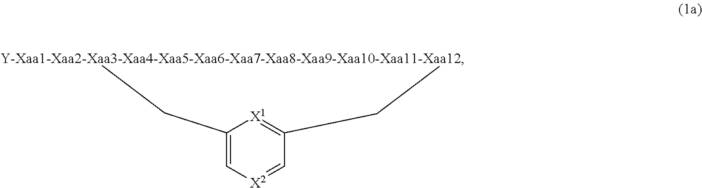

wherein, in formula (1a), the peptide sequence is drawn from left to right in N-terminal to C-terminal direction, and
   Y
      (i) is an N-terminal modification group A selected from the group consisting of R^{0a}—SO2-, R^{0a}—CO—, R^{0a}—NH—CO—, wherein
      R^{0a} is selected from the group consisting of (C₁-C₁₀)alkyl, (C₅-C₁₀)aryl, and (C₁-C₅)alkyl-(C₅-C₁₀)aryl, or
      (ii) comprises an effector E1, such as a chelator, wherein the effector E1 is covalently bound to Xaa1 if Xaa1 is present, or to Xaa2 if Xaa1 is absent and Xaa2 is present, or to Xaa3 if both Xaa1 and Xaa2 are absent,
      or
      (iii) is Z1, wherein Z1 comprises a linker moiety L1 and an effector E1, such as a chelator, wherein the linker moiety L1 covalently links the effector E1 to Xaa1 if Xaa1 is present, or to Xaa2 if Xaa1 is absent and Xaa2 is present, or to Xaa3 if both Xaa1 and Xaa2 are absent;
   Xaa1 is either present or absent, and if present is a residue of an aliphatic or polar L-amino acid;
   Xaa2 is either present or absent, wherein
      if Xaa2 is absent, Xaa1 is also absent and,
      if Xaa2 is present,
   (i) Xaa2 is a residue of an L-α-amino acid which is optionally N-methylated at the α-nitrogen atom, or,
   (ii) Xaa2 is a residue of an L-α-amino acid comprising, in addition to an amino group and a carboxy group attached to an α-C atom, a functional group FG1 forming a covalent linkage B1 with a functional group FG2 of Xaa11, wherein Xaa11 is a residue of an L-α-amino acid comprising, in addition to an amino group and a carboxy group attached to an α-C atom, the functional group FG2, wherein a bicyclic peptide of formula (1b) is formed

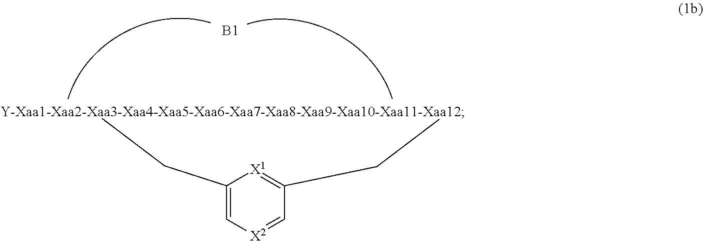

Xaa3 is a residue of an α-amino acid of formula (X):

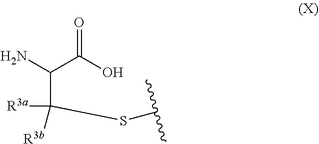

wherein
   R^{3a} and R^{3b} are each and independently selected from the group consisting of H and CH₃; and
   Xaa3 is preferably a residue of an L-α-amino acid such as Cys;
Xaa4 is a residue of an L-α-amino acid which is optionally N-methylated at the α-nitrogen atom;
Xaa5 is a residue of an amino acid which is optionally bound to Z3, wherein Xaa5 is a residue of an amino acid selected from the group consisting of N—(C₁-C₆)alkyl glycine, a D-α-amino acid, and an α,α-dialkylamino acid,
   wherein if Xaa5 comprises Z3,
   (i) Z3 is an effector E3, such as a chelator, Xaa5 is preferably a residue of an amino acid selected from the group consisting of 4-aminobutyl-glycine [Nlys], D-lys, (R)-ornithine [D-orn], (R)-2,4-diaminobutyric acid [D-dab], (R)-2,3-diaminopropionic acid [D-dap], and the chelator is attached to an N atom different from the α-nitrogen atom of any one of Nlys, D-lys, D-orn, D-dab, and D-dap, or
   (ii) Z3 comprises an effector E3, such as a chelator, and a linker moiety L3, Xaa5 is preferably a residue of an amino acid selected from the group consisting of Nlys, D-lys, D-orn, D-dab, and D-dap, and the linker moiety L3 is attached to an N atom different from the α-nitrogen atom of any one of Nlys, D-lys, D-orn, D-dab, and D-dap;
Xaa6 (i) is a residue of an amino acid which is selected from the group consisting of a polar L-α-amino acid an aromatic L-α-amino acid, an aliphatic α-amino acid, an S-alkylated cysteine, an oxidized form of an S-alkylated cysteine, and a residue of an amino acid according to formula (3):

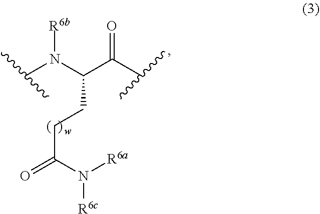

wherein
R^{6a} is selected from the group consisting of H a moiety comprising a —(C₅-C₁₀)aryl, (C₁-C₈)alkyl, and (C₁-C₅)alkyl-(C₅-C₁₀)aryl,
R^{6b} is selected from the group consisting of H and methyl,
R^{6c} is H or (C₁-C₆)alkyl, and
w is 0 or 1, or
(ii) is a residue of an L-α-amino acid comprising, in addition to an amino group and a carboxy group attached to an α-C atom, a functional group FG3 forming a covalent linkage B2 with a functional group FG4 of Xaa11, wherein Xaa11 is a residue of an α-amino acid comprising, in addition to an amino group and a carboxy group attached to an α-C atom, the functional group FG4, wherein a bicyclic peptide of formula (1c) is formed:

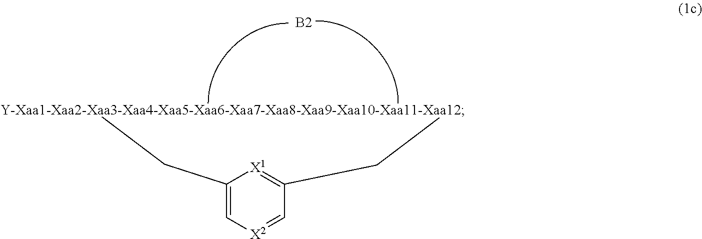

Xaa7 is a residue of an amino acid which is selected from the group consisting of an aromatic amino acid, such as a heteroaromatic L-α-amino acid and a substituted aromatic acid, such as a substituted heteroaromatic L-α amino acid;
Xaa8 is a residue of an amino acid which is selected from the group consisting of an L-α-amino acid and a cyclic α, α-dialkyl amino acid;
Xaa9 is a residue of an amino acid which is selected from the group consisting of Gly and an L-α-amino acid;
Xaa10 is a residue of a heteroaromatic L-α-amino acid;
Xaa11 (i) is a residue of an amino acid which is selected from the group consisting of Gly and an L-α-amino acid, wherein the L-α-amino acid is optionally bound to Z4, wherein Z4 comprises an effector E4, such as a chelator, and a linker moiety L4; or
   (ii) is a residue of an L-α-amino acid comprising, in addition to an amino group and a carboxy group attached to an α-C atom, the functional group FG2 forming the covalent linkage B1 with the functional group FG1 of Xaa2; or
   (iii) is a residue of an α-amino acid comprising, in addition to an amino group and a carboxy group attached to an α-C atom, the functional group FG4 forming the covalent linkage B2 with the functional group FG3 of Xaa6;
Xaa12 is a residue of an α-amino thiol of formula (XII):

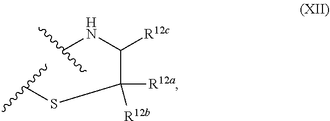

preferably of formula (XIIa)

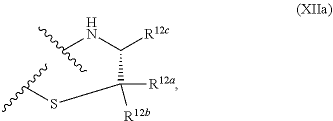

wherein
the NH of formula (XII) is bound to Xaa11;
R^{12a} and R^{12b} are each and independently selected from the group consisting of H and CH₃;
R^{12c} is selected from the group consisting of —CO—OH, CO—NH2, —CO—Z6 and —CH₂—Z6, wherein Z6 comprises a linker moiety L6 and an effector E6, such as a chelator; and
X¹ and X² are each and independently selected from the group consisting of C—H and N, and are both preferably C—H.

According to the present invention, each and any embodiment of the compound of the first aspect is also an embodiment of the peptide of the first aspect, and vice versa.

The definitions provided for Xaa1 to Xaa12 in the claims and the present specification have the meaning common in the art unless they have been specifically defined in the present specification. Insofar the definitions of Xaa1 to Xaa12 refer to expressions such as aliphatic, aromatic (e.g. heteroaromatic), polar, neutral, cyclic α,α-dialkyl amino acid, etc., reference is made to the definitions provided below in the specification and the examples given for these expressions.

Preferred embodiments of the broadest meanings used in connection with Xaa1 to Xaa12 are explained further below. Insofar the above preferred embodiments refer to “non-natural amino acids”, reference is made to the dependent claims and the following description which specify preferred non-natural amino acids for some of Xaa1 to Xaa12.

The problem underlying the present invention is also solved in a third aspect, which is also a first embodiment of the third aspect, by a compound selected from the group consisting of compound:
DOTA-PPAc-Gln-[Cys(3MeBn)-Glu-pro-Asp-Af3(Cpsu)-Leu-Thr-Trp-Ser-Cys]-NH₂ (3BP-4452, also referred to in the following description as DPI-4452) of the following formula:

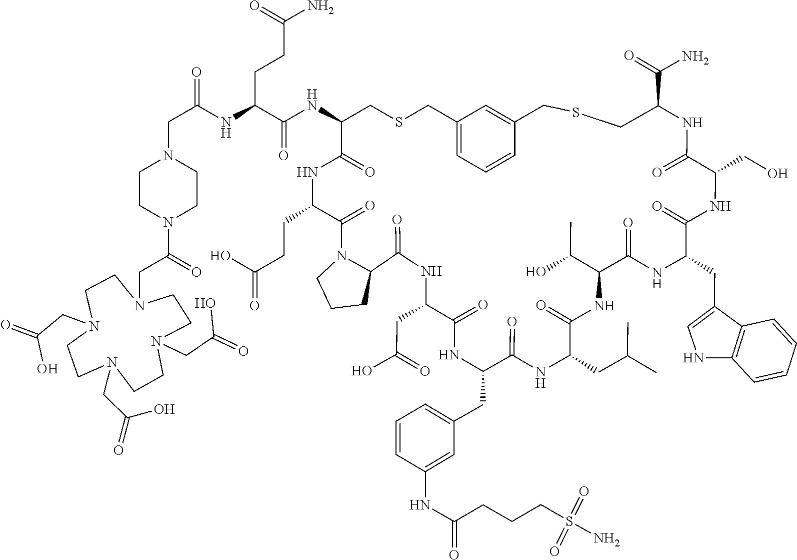 [SMILES: CC(C)C[C@@H]1NC(=O)[C@H](Cc2cccc(NC(=O)CCCS(N)(=O)=O)c2)NC(=O)[C@H](CC(=O)O)NC(=O)[C@H]2CCCN2C(=O)[C@H](CCC(=O)O)NC(=O)[C@@H](NC(=O)[C@H](CCC(N)=O)NC(=O)CN2CCN(C(=O)CN3CCN(CC(=O)O)CCN(CC(=O)O)CCN(CC(=O)O)CC3)CC2)CSCc2cccc(c2)CSC[C@@H](C(N)=O)NC(=O)[C@H](CO)NC(=O)[C@H](Cc2c[nH]c3ccccc23)NC(=O)[C@H]([C@@H](C)O)NC1=O]

compound DOTA-Gln-[Cys(3MeBn)-Glu-pro-Asp-Aph(SaPr)-Leu-Thr-Trp-Ser-Cys]-NH₂ (3BP-4501, also referred to in the following description as DPI-4501) of the following formula:

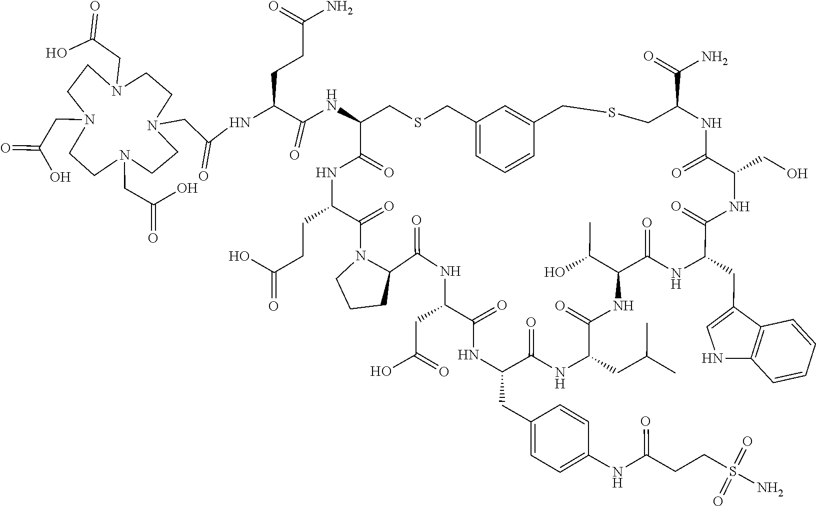 [SMILES: CC(C)C[C@@H]1NC(=O)[C@H](Cc2ccc(NC(=O)CCS(N)(=O)=O)cc2)NC(=O)[C@H](CC(=O)O)NC(=O)[C@H]2CCCN2C(=O)[C@H](CCC(=O)O)NC(=O)[C@@H](NC(=O)[C@H](CCC(N)=O)NC(=O)CN2CCN(CC(=O)O)CCN(CC(=O)O)CCN(CC(=O)O)CC2)CSCc2cccc(c2)CSC[C@@H](C(N)=O)NC(=O)[C@H](CO)NC(=O)[C@H](Cc2c[nH]c3ccccc23)NC(=O)[C@H]([C@@H](C)O)NC1=O]

compound DOTA-{Glu-[Cys(3MeBn)-Glu-pro-Asp-Af3(Cpsu)-Leu-Thr-Trp-Dap}-Cys]-NH₂ (3BP-4503, also referred to in the following description as DPI-4503) of the following formula:

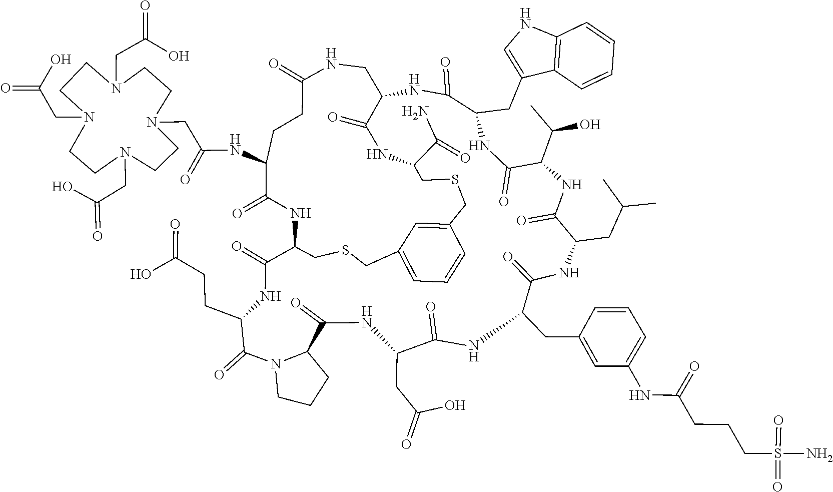 [SMILES: CC(C)C[C@@H]1NC(=O)[C@H](Cc2cccc(NC(=O)CCCS(N)(=O)=O)c2)NC(=O)[C@H](CC(=O)O)NC(=O)[C@H]2CCCN2C(=O)[C@H](CCC(=O)O)NC(=O)[C@@H]2CSCc3cccc(c3)CSC[C@@H](C(N)=O)NC(=O)[C@H](CNC(=O)CC[C@H](NC(=O)CN3CCN(CC(=O)O)CCN(CC(=O)O)CCN(CC(=O)O)CC3)C(=O)N2)NC(=O)[C@H](Cc2c[nH]c3ccccc23)NC(=O)[C@H]([C@@H](C)O)NC1=O]

The compound of the first aspect, including any embodiment thereof, the peptide of the second aspect, including any embodiment thereof, and the compound of the third aspect, including any embodiment thereof, are also referred to as the compound of the invention.

The problem underlying the present invention is also solved in a fourth aspect which is also a first embodiment of the fourth aspect, by the compound of the first aspect, the peptide of the second aspect or the compound of the third aspect, including each and any embodiment thereof, for the diagnosis of a disease.

The problem underlying the present invention is also solved in a fifth aspect which is also a first embodiment of the fifth aspect, by the compound of the first aspect, the peptide of the second aspect or the compound of the third aspect, including each and any embodiment thereof, for use in a method for the treatment of a disease.

The problem underlying the present invention is also solved in a sixth aspect which is also a first embodiment of the sixth aspect, by the compound of the first aspect, the peptide of the second aspect and the compound of the third aspect, including each and any embodiment thereof, for use in a method for the identification of a subject, wherein the subject is likely to respond or likely not to respond to a treatment of a disease, wherein the method for the identification of a subject comprises carrying out a method of diagnosis using the compound of the first aspect, the peptide of the second aspect or the compound of the third aspect, including each and any embodiment thereof.

The problem underlying the present invention is also solved in a seventh aspect which is also a first embodiment of the seventh aspect, by the compound of the first aspect, the peptide of the second aspect or the compound of the third aspect, including each and any embodiment thereof, for use in a method for the selection of a subject from a group of subjects, wherein the subject is likely to respond or likely not to respond to a treatment of a disease, wherein the method for the selection of a subject from a group of subjects comprises carrying out a method of diagnosis using the compound of the first aspect, the peptide of the second aspect, or the compound of the third aspect, including each and any embodiment thereof.

The problem underlying the present invention is also solved in an eighth aspect which is also a first embodiment of the eighth aspect, by the compound of the first aspect, the peptide of the second aspect or the compound of the third aspect, including each and any embodiment thereof, for use in a method for the stratification of a group of subjects into subjects which are likely to respond to a treatment of a disease, and into subjects which are not likely to respond to a treatment of a disease, wherein the method for the stratification of a group of subjects comprises carrying out a method of diagnosis using the compound of the first aspect, the peptide of the second aspect or the compound of the third aspect, including any embodiment thereof.

The problem underlying the present invention is solved in a ninth aspect by a composition, preferably a pharmaceutical composition, wherein the composition comprises the compound of the first aspect, the peptide of the second aspect and/or the compound of the third aspect, including any embodiment thereof, and a pharmaceutically acceptable excipient.

The problem underlying the present invention is solved in a tenth aspect by a kit comprising the compound of the first aspect, the peptide of the second aspect and/or the compound of the third aspect, including any embodiment thereof, one or more optional excipient(s) and optionally one or more device(s), whereby the device(s) is/are selected from the group comprising a labeling device, a purification device, a handling device, a radioprotection device, an analytical device or an administration device.

### 1. DEFINITIONS

The term “peptide” refers to a compound comprising a continuous sequence of at least three amino acids linked to each other via peptide linkages. The term “peptide linkage” in this connection is meant to encompass (backbone) amide bonds as well as modified linkages, which can be obtained if non-natural amino acids are introduced in the peptidic sequence. In this case, the modified linkage replaces the (backbone) amide bond which is formed in the continuous peptide sequence by reacting the amino group and the carboxyl group of two amino acid residues. For instance, the modified linkage may be an ester, an ether, thioether, a thiourea, a carbamate, or a triazole linkage (as described further below). Preferably, the amino acids forming the continuous peptide sequence are linked to each other via backbone amide bonds. The peptide may be linear or branched, e.g., cyclic. Here, the amino acids include both naturally occurring amino acids as well as non-natural (synthetic) amino acids, as described further below.

The term “C-terminal” as used herein refers to the C-terminal end of a peptide chain. The C-terminal amino acid residue of a peptide sequence is the last amino acid of the sequence which is bound via its amino group to the peptide chain wherein its carboxy group is not involved in binding to the peptide chain. The carboxy group of the C-terminal amino acid residue may be a free carboxy group or a group derived from the carboxy group like, for instance, an amide or ester group. For instance, binding of group “X” to the carboxy group of a C-terminal amino acid residue “Xaa” yields an ester or amide-type structural element—C(O)—X, wherein the carbonyl group is derived from the acid group of Xaa.

The term “N-terminal” as used herein refers to the N-terminal end of a peptide chain. The N-terminal amino acid residue of a peptide sequence is the first amino acid of the sequence which is bound via its carboxy group to the peptide chain wherein its amino group is not involved in binding to the peptide chain. The amino group of the N-terminal residue is either unmodified or modified. Modification of the “N-terminal” amino acid residue means that a covalent bond is formed between the amino group in the main chain (backbone) of the amino acid residue and the binding partner (which replaces one hydrogen atom), wherein this linkage is typically selected from the group consisting of amide, urea, carbamate, thiourea, sulfonamide and alkylamine (—CH₂—N—) linkages.

In an embodiment and as preferably used herein, a linkage is an attachment of two atoms of two independent moieties. A preferred linkage is a chemical bond or a plurality of chemical bonds. More preferably, a chemical bond is a covalent bond or a plurality of chemical bonds. Most preferably, the linkage is a covalent bond or a coordinate bond. As preferably used herein, an embodiment of a coordinate bond is a bond or group of bonds as realized when a metal is bound by a chelator. Depending on the type of atoms linked and their atomic environment different types of linkages are created. These types of linkage are defined by the type of atom arrangements created by the linkage.

For instance, the linking of a moiety comprising an amine with a moiety comprising a carboxylic acid leads to a linkage named “amide” (which is also referred to as amide linkage, —CO—N—, —N—CO—). It will be acknowledged by a person skilled in the art that this and the following examples of creating linkages are only prototypical examples and are by no means limiting the scope of the instant application. It will be acknowledged by a person in the art that the linking of a moiety comprising an isothiocyanate with a moiety comprising an amine leads to thiourea (which is also referred to as a thiourea linkage, —N—CS—N—), and linking of a moiety comprising a C atom with a moiety comprising a thiol-group (—C—SH) leads to thioether (which is also referred to as a thioether linkage, —C—S—C—). A linkage as preferably used in connection with the chelator and linker of the invention and their characteristic type of atom arrangement is presented in Table 4.

TABLE 4

| Linkage | Characteristic atom arrangement |
|---|---|
| Amide | 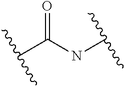 [SMILES: *NC(*)=O] |
| Ether | 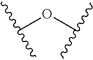 [SMILES: *O*] |
| Thioether | 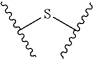 [SMILES: *S*] |
| Carbamate | 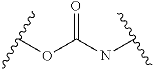 [SMILES: *NC(=O)O*] |
| Thiourea | 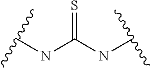 [SMILES: *NC(=S)N*] |
| Triazole | 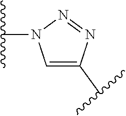 [SMILES: *c1cn(*)nn1] |
| Pyrazine | 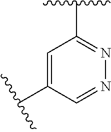 [SMILES: *c1cnnc(*)c1] or 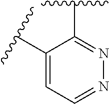 [SMILES: *c1ccnnc1*] |
| Dihydro-pyrazine | 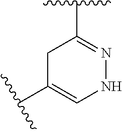 [SMILES: *C1=CNN=C(*)C1] or 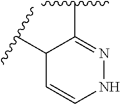 [SMILES: *C1=NNC=CC1*] and isomers |

Examples of reactive groups which, in some embodiments of the invention, are used in the formation of linkages between the effector, e.g., a chelator preferably comprising a chelated nuclide, more preferably a chelated diagnostically and/or therapeutically active radionuclide, and the remaining of the molecule are summarized in Table 5. It will, however, be understood by a person skilled in the art that neither the linkages which may be realized in embodiments for the formation of the conjugates of the invention are limited to the ones of Table 5 nor the reactive groups forming such linkages.

TABLE 5

| first | second | (type of) |
|---|---|---|
| reactive group | reactive group | linkage |
| amino | carboxylic acid | amide |
| amino | activated carboxylic acid | amide |
| carboxylic acid | amino | amide |
| sulfhydryl | Michael acceptor | thioether |
| | (e.g. Maleimide) | |
| bromo | sulfhydryl | thioether |
| isothiocyanate | amino | thiourea |
| azide | alkyne | triazole |
| isocyanate | amino | carbamate |

The following are reactive groups and functionalities which are utilized or amenable of forming linkages between moieties or structures as used in embodiments of the conjugate of the invention: primary or secondary amino, carboxylic acid, activated carboxylic acid, chloro, bromo, iodo, sulfhydryl, hydroxyl, sulfonic acid, activated sulfonic acid, sulfonic acid esters like mesylate or tosylate, Michael acceptors, strained alkenes like trans cyclooctene, isocyanate, isothiocyanate, azide, alkyne and tetrazine.

As preferably used herein, the term “activated carboxylic acid” refers to a carboxylic acid group with the general formula —CO—X, wherein X is a leaving group. For example, activated forms of a carboxylic acid group may include, but are not limited to, acyl chlorides, symmetrical or unsymmetrical anhydrides, and esters. In some embodiments, the activated carboxylic acid group is an ester with pentafluorophenol, nitrophenol, benzotriazole, azabenzotriazole, thiophenol or N-hydroxysuccinimide (NHS) as leaving group.

As preferably used herein the term “sulfonic acid ester” refers to a functional group which is characterized by —O—SO₂—R, wherein R is preferably (C₁-C₈)alkyl or aryl. Sulfonic acid esters are similarly to halogens typical leaving groups in nucleophilic substitutions.

“Michael acceptors” comprise at least one unsaturated, non-aromatic C—C-bond which is substituted by at least one electron-withdrawing group, preferably CO—, CN, NO₂ and SO₂—. These Michael acceptors are substrates for the conjugate addition of many nucleophilic partners in the well-known Michael addition reaction. Prominent examples are acrylic acids, maleimides or vinyl sulfones.

To the extent it is referred in the instant application to a range indicated by a lower integer and a higher integer such as, for example, 1-4, such range is a representation of the lower integer, the higher integer and any integer between the lower integer and the higher integer. Insofar, the range is actually an individualized disclosure of said integer. In said example, the range of 1-4 thus means 1, 2, 3 and 4.

In an embodiment, and as preferably used herein, “(C₁-C₈)alkyl” refers to a saturated or unsaturated, straight-chain, cyclic or branched hydrocarbon group having from 1 to 8 carbon atoms. Representative (C₁-C₈)alkyl groups include, but are not limited to, any of methyl, ethyl, n-propyl, isopropyl, n-butyl, isobutyl, sec-butyl, tert-butyl, n-pentyl, 2-pentyl, 2-methyl-butyl, 3-methyl-butyl, 3-pentyl, 3-methyl-but-2-yl, 2-methyl-but-2-yl, 2,2-dimethylpropyl, n-hexyl, 2-hexyl, 2-methyl-pentyl, 3-methyl-pentyl, 4-methyl-pentyl, 3-hexyl, 2-ethyl-butyl, 2-methyl-pent-2-yl, 2,2-dimethyl-butyl, 3,3-dimethyl-butyl, 3-methyl-pent-2-yl, 4-methyl-pent-2-yl, 2,3-dimethyl-butyl, 3-methyl-pent-3-yl, 2-methyl-pent-3-yl, 2,3-dimethyl-but-2-yl, 3,3-dimethyl-but-2-yl, n-heptyl, 2-heptyl, 2-methyl-hexyl, 3-methyl-hexyl, 4-methyl-hexyl, 5-methyl-hexyl, 3-heptyl, 2-ethyl-pentyl, 3-ethyl-pentyl, 4-heptyl, 2-methyl-hex-2-yl, 2,2-dimethyl-pentyl, 3,3-dimethyl-pentyl, 4,4-dimethyl-pentyl, 3-methyl-hex-2-yl, 4-methyl-hex-2-yl, 5-methyl-hex-2-yl, 2,3-dimethyl-pentyl, 2,4-dimethyl-pentyl, 3,4-dimethyl-pentyl, 3-methyl-hex-3-yl, 2-ethyl-2-methyl-butyl, 4-methyl-hex-3-yl, 5-methyl-hex-3-yl, 2-ethyl-3-methyl-butyl, 2,3-dimethyl-pent-2-yl, 2,4-dimethyl-pent-2-yl, 3,3-dimethyl-pent-2-yl, 4,4-dimethyl-pent-2-yl, 2,2,3-trimethyl-butyl, 2,3,3-trimethyl-butyl, 2,3,3-trimethyl-but-2-yl, n-octyl, 2-octyl, 2-methyl-heptyl, 3-methyl-heptyl, 4-methyl-heptyl, 5-methyl-heptyl, 6-methyl-heptyl, 3-octyl, 2-ethyl-hexyl, 3-ethyl-hexyl, 4-ethyl-hexyl, 4-octyl, 2-propyl-pentyl, 2-methyl-hept-2-yl, 2,2-dimethyl-hexyl, 3,3-dimethyl-hexyl, 4,4-dimethyl-hexyl, 5,5-dimethyl-hexyl, 3-methyl-hept-2-yl, 4-methyl-hept-2-yl, 5-methyl-hept-2-yl, 6-methyl-hept-2-yl, 2,3-dimethyl-hex-1-yl, 2,4-dimethyl-hex-1-yl, 2,5-dimethyl-hex-1-yl, 3,4-dimethyl-hex-1-yl, 3,5-dimethyl-hex-1-yl, 3,5-dimethyl-hex-1-yl, 3-methyl-hept-3-yl, 2-ethyl-2-methyl-1-yl, 3-ethyl-3-methyl-1-yl, 4-methyl-hept-3-yl, 5-methyl-hept-3-yl, 6-methyl-hept-3-yl, 2-ethyl-3-methyl-pentyl, 2-ethyl-4-methyl-pentyl, 3-ethyl-4-methyl-pentyl, 2,3-dimethyl-hex-2-yl, 2,4-dimethyl-hex-2-yl, 2,5-dimethyl-hex-2-yl, 3,3-dimethyl-hex-2-yl, 3,4-dimethyl-hex-2-yl, 3,5-dimethyl-hex-2-yl, 4,4-dimethyl-hex-2-yl, 4,5-dimethyl-hex-2-yl, 5,5-dimethyl-hex-2-yl, 2,2,3-trimethyl-pentyl, 2,2,4-trimethyl-pentyl, 2,3,3-trimethyl-pentyl, 2,3,4-trimethyl-pentyl, 2,4,4-trimethyl-pentyl, 3,3,4-trimethyl-pentyl, 3,4,4-trimethyl-pentyl, 2,3,3-trimethyl-pent-2-yl, 2,3,4-trimethyl-pent-2-yl, 2,4,4-trimethyl-pent-2-yl, 3,4,4-trimethyl-pent-2-yl, 2,2,3,3-tetramethyl-butyl, 3,4-dimethyl-hex-3-yl, 3,5-dimethyl-hex-3-yl, 4,4-dimethyl-hex-3-yl, 4,5-dimethyl-hex-3-yl, 5,5-dimethyl-hex-3-yl, 3-ethyl-3-methyl-pent-2-yl, 3-ethyl-4-methyl-pent-2-yl, 3-ethyl-hex-3-yl, 2,2-diethyl-butyl, 3-ethyl-3-methyl-pentyl, 4-ethyl-hex-3-yl, 5-methyl-hept-3-yl, 2-ethyl-3-methyl-pentyl, 4-methyl-hept-4-yl, 3-methyl-hept-4-yl, 2-methyl-hept-4-yl, 3-ethyl-hex-2-yl, 2-ethyl-2-methyl-pentyl, 2-isopropyl-pentyl, 2,2-dimethyl-hex-3-yl, 2,2,4-trimethyl-pent-3-yl and 2-ethyl-3-methyl-pentyl. A (C₁-C₈)alkyl group can be unsubstituted or substituted with one or more groups, including, but not limited to, (C₁-C₈)alkyl, —O—[(C₁-C₈)alkyl], -aryl, —CO—R′, —O—CO—R′, —COOR′, —CONH₂, —CONHR′, —CONR′₂, —NH—CO—R′, —SO₂—R′, —SO—R′, —OH, -halogen, —N₃, —NH₂, —NHR′, —NR′₂ and —CN; where each R′ is independently selected from —(C₁-C₈)alkyl and aryl.

The terms “(C₁-C₄)alkyl”, “(C₁-C₅)alkyl”, “(C₂-C₅)alkyl”, “(C₁-C₆)alkyl”, and “(C₁-C₁₀)alkyl” are in their meaning analogous to the term “(C₁-C₈)alkyl” but differ in the indicated range of number of C atoms. However, these alkyl groups can also be substituted with one or more groups, including, but not limited to, (C₁-C₈)alkyl, —O—[(C₁-C₈)alkyl], -aryl, —CO—R′, —O—CO—R′, —COOR′, —CONH₂, —CONHR′, —CONR′₂, —NH—CO—R′, —SO₂—R′, —SO—R′, —OH, -halogen, —N₃, —NH₂, —NHR′, —NR′₂ and —CN; where each R′ is independently selected from —(C₁-C₈)alkyl and aryl.

In an embodiment, and as preferably used herein, “(C₃-C₇)cycloalkyl” refers to a saturated or unsaturated, or branched hydrocarbon group comprising a carbocyclic structure having from 3 to 7 carbon atoms.

In an embodiment, and as preferably used herein, “(C₃-C₈)cycloalkyl” refers to a saturated or unsaturated, or branched hydrocarbon group comprising a carbocyclic structure having from 3 to 8 carbon atoms.

All groups which are termed “cycloalkyl”, independent of their number of C atoms, can also be substituted with one or more groups, including, but not limited to, (C₁-C₈)alkyl, —O—[(C₁-C₈)alkyl], -aryl, —CO—R′, —O—CO—R′, —COOR′, —CONH₂, —CONHR′, —CONR′₂, —NH—CO—R′, —SO₂—R′, —SO—R′, —OH, -halogen, —N₃, —NH₂, —NHR′, —NR′₂ and —CN; where each R′ is independently selected from —(C₁-C₈)alkyl and aryl.

In an embodiment, and as preferably used herein, “aryl” refers to a group comprising an aromatic system wherein the aromatic system is carbocyclic or heterocyclic, preferably consists of 5 to 10 C- or hetero-atoms in the ring and the aryl group can be unsubstituted or substituted with one or more groups including, but not limited to, —(C₁-C₈)alkyl, —O—[(C₁-C₈)alkyl], -aryl, —CO—R′, —O—CO—R′, —CO—OR′, —CO—NH₂, —CO—NHR′, —CO—NR′₂, —NH—CO—R′, —SO₂—R′, —SO—R′, —OH, -halogen, —N₃, —NH₂, —NHR′, —NR′₂ and —CN; wherein each R′ is independently selected from —(C₁-C₈)alkyl and aryl.

In an embodiment, and as preferably used herein, “heterocyclyl” refers to a heterocyclic aromatic or non-aromatic group. Examples of heterocyclic groups include, but are not limited to, furane, thiophene, pyridine, pyrimidine, benzothiophene, benzofurane, quinoline, piperidine, piperazine, morpholine, oxirane, tetrahydrofuran and pyrollidine.

In an embodiment, and as preferably used herein, “(C₅-C₁₀)heterocyclyl” refers to a heterocyclic aromatic or non-aromatic group consisting of 5 or 10 ring atoms wherein at least one atom is different from carbon, including, for example, nitrogen, sulfur or oxygen. A heterocyclic aromatic group can be unsubstituted or substituted with one or more groups including, but not limited to, —(C₁-C₈)alkyl, —O—[(C₁-C₈)alkyl], -aryl, —CO—R′, —O—CO—R′, —CO—OR′, —CO—NH₂, —CO—NHR′, —CO—NR′₂, —NH—CO—R′, —SO₂—R′, —SO—R′, —OH, -halogen, —N₃, —NH₂, —NHR′, —NR′₂ and —CN; wherein each R′ is independently selected from —(C₁-C₈)alkyl and aryl.

In an embodiment, and as preferably used herein, “heteroaryl” refers to a heterocyclic aromatic group. Examples of heteroaryl groups include, but are not limited to, furane, thiophene, pyridine, pyrimidine, benzothiophene, benzofurane, and quinoline.

In an embodiment, and as preferably used herein, “(C₅-C₁₀)heteroaryl” refers to a heterocyclic aromatic group consisting of 5 or 10 ring atoms wherein at least one atom is different from carbon, including, for example, nitrogen, sulfur or oxygen. A heterocyclic aromatic group can be unsubstituted or substituted with one or more groups including, but not limited to, —(C₁-C₈)alkyl, —O—[(C₁-C₈)alkyl], -aryl, —CO—R′, —O—CO—R′, —CO—OR′, —CO—NH₂, —CO—NHR′, —CO—NR′₂, —NH—CO—R′, —SO₂—R′, —SO—R′, —OH, -halogen, —N₃, —NH₂, —NHR′, —NR′₂ and —CN; wherein each R′ is independently selected from —(C₁-C₈)alkyl and aryl.

In an embodiment, and as preferably used herein, “(C₁-C₅)alkyl-(C₅-C₁₀)aryl” refers to a group (C₁-C₅)alkyl covalently bound to a group —(C₅-C₁₀)aryl.

In an embodiment, and as preferably used herein, “(C₃-C₇)cycloalkyl-(C₅-C₁₀)aryl” is a cycloalkyl group consisting of 3, 4, 5, 6, or 7 C atoms which is bound to a (C₅-C₁₀)aryl group.

Compounds of the invention typically contain amino acid sequences as provided herein. The term “amino acid” as used herein refers to a compound that contains or is derived from a compound containing at least one amino group and at least one acidic group, preferably a carboxy group. The distance between amino group and acidic group is not particularly limited. If not other specified, α-, β-, γ, δ-, and ε-amino acids are suitable, however, in many cases α-amino acids and especially α-amino carboxylic acids are particularly preferred. The term “amino acid” encompasses both naturally occurring amino acids such as the naturally occurring proteinogenic amino acids, as well as synthetic amino acids that are not found in nature (“non-natural amino acids”). The term “residue” or “residue of an amino acid” is used to characterize amino acids bonded to adjacent amino acids or moieties, which differ from the amino acids from which they are derived only by the structural elements responsible for bonding to adjacent amino acids or moieties.

Conventional amino acids, also referred to as “natural amino acids” are identified according to their standard three-letter codes and one-letter abbreviations, as set forth in Table 6.

TABLE 6
Natural amino acids and their abbreviations

| | 3-letter | 1-letter |
|---|---|---|
| Amino acid | abbreviation | abbreviation |
| Alanine | Ala | A |
| Arginine | Arg | R |
| Asparagine | Asn | N |
| Aspartic acid | Asp | D |
| Cysteine | Cys | C |
| Glutamic acid | Glu | E |
| Glutamine | Gln | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

Non-conventional amino acids, also referred to as “non-natural amino acids”, are any kind of non-oligomeric compound which comprises an amino group and a carboxylic group and is not a conventional amino acid. The size of non-natural amino acids is not specifically limited and may, e.g., correspond to a molecular weight of up to 500 g/mol, such as up to 400 g/mol.

Examples of non-natural amino acids and other building blocks as used for the construction of compounds of the invention are identified according to their abbreviation or name found in Table 7. The structures of some building blocks are depicted with an exemplary reagent for introducing the building block into the peptide (e.g., as carboxylic acid like) or these building blocks are shown as residue which is completely attached to another structure like a peptide or amino acid. The structures of the amino acids are shown as explicit amino acids and not as residues of the amino acids how they are presented after implementation in the peptide sequence. Some larger chemical moieties consisting of more than one moiety are also shown.

TABLE 7
Abbreviation, name and structure of non-natural amino-acid and other building
blocks and chemical moieties

| Abbreviation | Name | Structure |
|---|---|---|
| 1MW | D/L-1-Methyltryptophane | 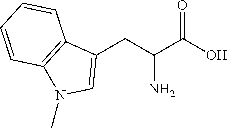 [SMILES: Cn1cc(CC(N)C(=O)O)c2ccccc21] |
| 1Ni | 3-(1-Naphthyl)alanine | 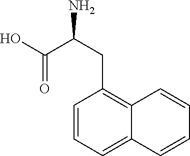 [SMILES: N[C@@H](Cc1cccc2ccccc12)C(=O)O] |
| 2Lut | 2,6-Lutidylidene (derived from 2,6-lutidine) | 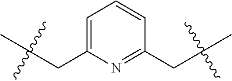 [SMILES: *Cc1cccc(C*)n1] |
| 2Py6SaNH | 6-(Sulfamoylamino)pyridine-2- caboxylic acid | 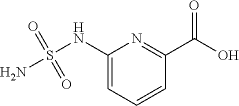 [SMILES: NS(=O)(=O)Nc1cccc(C(=O)O)n1] |
| 2Quyl | 2-Quinolinyl | 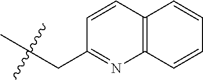 [SMILES: *Cc1ccc2ccccc2n1] |
| 2Ta5Sa | 5-Sulfamoyl-thiophene-2- carboxylic acid | 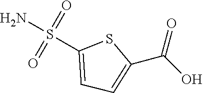 [SMILES: NS(=O)(=O)c1ccc(C(=O)O)s1] |
| 2Thz | 1,3-Thiazole-2-carboxylic acid | 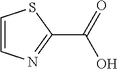 |
| 3Lut | 3,5-Lutidylidene (derived from 3,5-lutidine) | 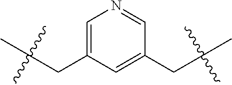 [SMILES: *Cc1cncc(C*)c1] |
| 3MeBn | 3-Methylbenzylidene | 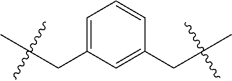 [SMILES: *Cc1cccc(C*)c1] |
| 3MeBnSpa | 3-Metyhlbenzylmercapto- propionic acid | 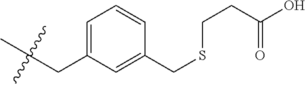 [SMILES: *Cc1cccc(CSCCC(=O)O)c1] |
| 3MSaBz | 3-(Sulfamoylmethyl)benzoic acid | 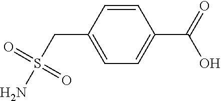 [SMILES: NS(=O)(=O)Cc1ccc(C(=O)O)cc1] |
| 3OHPr | 3-Hydroxypropionic acid | 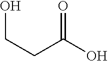 |
| 3SaBz | 3-Sulfamoylbenzoic Acid | 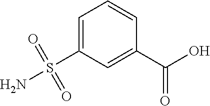 [SMILES: NS(=O)(=O)c1cccc(C(=O)O)c1] |
| 3Ta5Sa | 5-Sulfamoylthiophene-3- carboxylic acid | 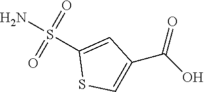 [SMILES: NS(=O)(=O)c1cc(C(=O)O)cs1] |
| 4Amc | 4-trans- Aminomethylcyclohexane carboxylic acid/Tranexamic acid | 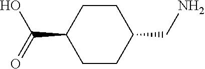 [SMILES: NC[C@H]1CC[C@H](C(=O)O)CC1] |
| 4OHPhp | 4-Hydroxyphenylpropionic acid | 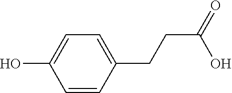 [SMILES: O=C(O)CCc1ccc(O)cc1] |
| 4Pya | 4-Pyridylacetic acid | 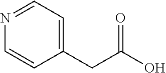 |
| 4SaBz | 4-Sulfamoyl-benzoic acid | 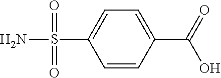 [SMILES: NS(=O)(=O)c1ccc(C(=O)O)cc1] |
| 4SaPy2Ac | (4-Sulfamoyl-pyrazol-1-yl)- acetic acid | 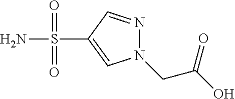 [SMILES: NS(=O)(=O)c1cnn(CC(=O)O)c1] |
| 5Clw | 5-Chloro-tryptophane | 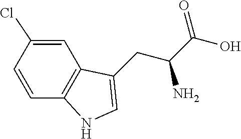 [SMILES: N[C@@H](Cc1c[nH]c2ccc(Cl)cc12)C(=O)O] |
| 5SaPyr2 | 5-sulfamoylpyridine-2- carboxylic acid | 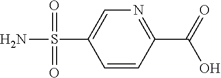 [SMILES: NS(=O)(=O)c1ccc(C(=O)O)nc1] |
| 6SaPyr3 | 6-sulfamoylpyridine-3- carboxylic acid | 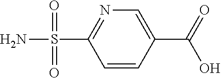 [SMILES: NS(=O)(=O)c1ccc(C(=O)O)cn1] |
| 7MW | D/L-7-Methyltryptophane | 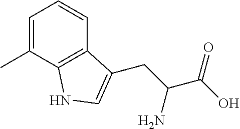 [SMILES: Cc1cccc2c(CC(N)C(=O)O)c[nH]c12] |
| 7Nw | 7-Aza-tryptophane | 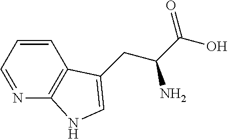 [SMILES: N[C@@H](Cc1c[nH]c2ncccc12)C(=O)O] |
| Ac | Acetic acid | 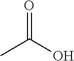 |
| Adp | Adipic acid | 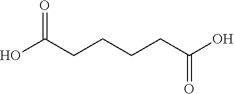 |
| Aeg | aminoethylglycine | 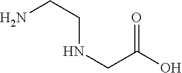 |
| AET | 2-Aminoethanethiol | 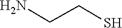 |
| Af3 | 3-Amino-phenylalanine | 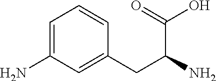 [SMILES: Nc1cccc(C[C@H](N)C(=O)O)c1] |
| AF488 | Alexa Fluor 488 Dye | 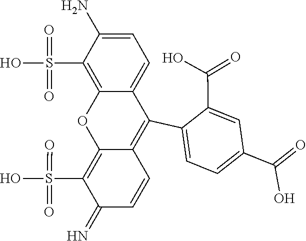 [SMILES: N=c1ccc2c(-c3ccc(C(=O)O)cc3C(=O)O)c3ccc(N)c(S(=O)(=O)O)c3oc-2c1S(=O)(=O)O] |
| AGLU | 1-amino-1-deoxy-D-glucitol | 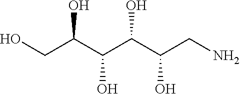 [SMILES: NC[C@H](O)[C@@H](O)[C@H](O)[C@H](O)CO] |
| Aib | 2-Amino-isobutyric acid | 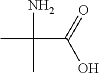 |
| Aic | 2-Aminoindane-2-carboxylic acid | 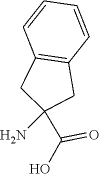 [SMILES: NC1(C(=O)O)Cc2ccccc2C1] |
| Aml | (S)-α-Methyl -leucine | 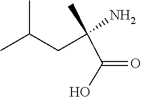 |
| APAc | 2-(4-(Amino)piperidin-1- yl)acetic acid | 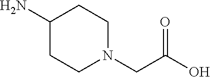 [SMILES: NC1CCN(CC(=O)O)CC1] |
| Apc | 4-amino-piperidine-4-carboxylic acid | 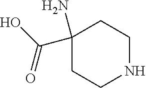 |
| Ape | Pentane-1,5-diamine | 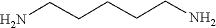 |
| Ape-DOTA | 4-[[(5-Amino-pentylcarbamoyl)- methyl]-7,10-bis- carboxymethyl-1,4,7,10tetraaza- cyclododec-1-yl]-acetic acid | 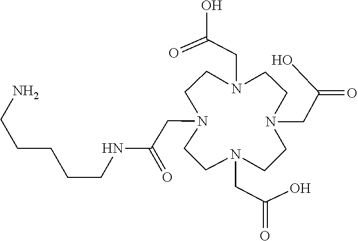 [SMILES: NCCCCCNC(=O)CN1CCN(CC(=O)O)CCN(CC(=O)O)CCN(CC(=O)O)CC1] |
| Apg | N-3-Aminopropyl-glycine | 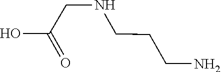 |
| Aph | 4-Aminophenylalanine | 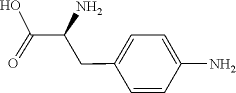 [SMILES: Nc1ccc(C[C@H](N)C(=O)O)cc1] |
| Apr | 1,3-Diaminopropane | 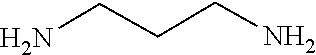 |
| Ava | 5-Amino-pentanoic acid | 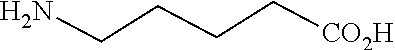 |
| Aytr | 2-{4-[(tert-butoxy)carbamoyl]- 1H-1,2,3-triazol-1-yl}acetic acid | 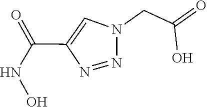 [SMILES: O=C(O)Cn1cc(C(=O)NO)nn1] |
| Bal | β-Alanine | 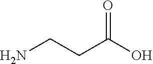 |
| Bio | D(+)-Biotin | 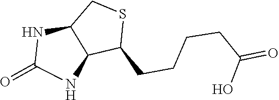 [SMILES: O=C(O)CCCC[C@@H]1SC[C@@H]2NC(=O)N[C@@H]21] |
| Bip | (S)-Biphenylalanine | 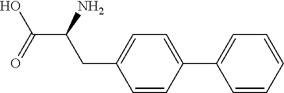 [SMILES: N[C@@H](Cc1ccc(-c2ccccc2)cc1)C(=O)O] |
| Bta | 3-Benzothienyl alanine | 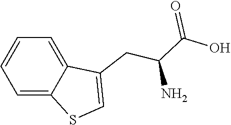 [SMILES: N[C@@H](Cc1csc2ccccc12)C(=O)O] |
| Btda | 1,1,3-Trioxo-1,2,3,4-tetrahydro- 1λ⁶-benzo[1,2,4]thiadiazine-7- carboxylic acid | 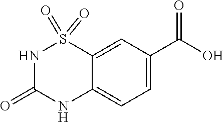 [SMILES: O=C1Nc2ccc(C(=O)O)cc2S(=O)(=O)N1] |
| Btz | 1,1,3-Trioxo-2,3-dihydro-1H- 1λ⁶-benzo[d]isothiazole-5- carboxylic acid | 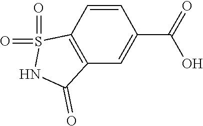 [SMILES: O=C(O)c1ccc2c(c1)C(=O)NS2(=O)=O] |
| Bz | benzoyl | 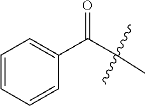 [SMILES: *C(=O)c1ccccc1] |
| Bzl | benzyl | 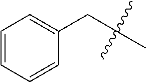 [SMILES: *Cc1ccccc1] |
| CCprAc | 1-Cyano-1- cyclopropanecarboxylic acid | 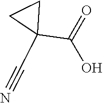 |
| Cha | Cyclohexylalanine | 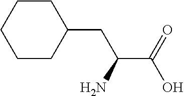 [SMILES: N[C@@H](CC1CCCCC1)C(=O)O] |
| CImPy | 6-cyanoimidazo[1,2-a]pyridine- 3-carboxylic acid | 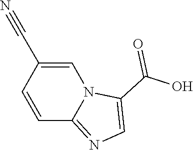 [SMILES: N#Cc1ccc2ncc(C(=O)O)n2c1] |
| Cmp | 4-Carboxymethyl-piperidine | 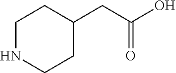 |
| CMPy | 5-cyano-1-methyl-1H-pyrazole- 4-carboxylic acid | 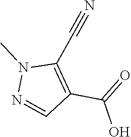 |
| Cp | Cyclopentane carboxylic acid | 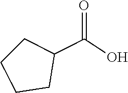 |
| Cpsu | 4-Sulfamoyl-butyric acid | 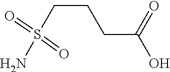 |
| Cshx | 4-sulfamoylcyclohexane-1- carboxylic acid | 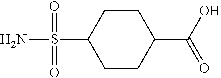 [SMILES: NS(=O)(=O)C1CCC(C(=O)O)CC1] |
| Cy5SO3 | Cy5 dye (mono SO3) | 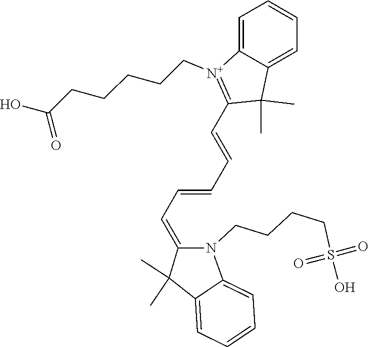 [SMILES: CC1(C)C(/C=C/C=C/C=C2\N(CCCCS(=O)(=O)O)c3ccccc3C2(C)C)=[N+](CCCCCC(=O)O)c2ccccc21] |
| Cya | (R)-Cysteic acid | 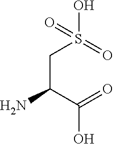 |
| Cyhx | 4-Hydroxycarbamoyl- cyclohexanecarboxylic acid | 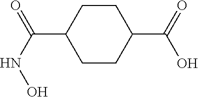 [SMILES: O=C(O)C1CCC(C(=O)NO)CC1] |
| Cys(2Lut) | | 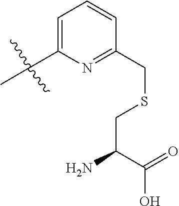 [SMILES: *c1cccc(CSC[C@H](N)C(=O)O)n1] |
| Cys(3Lut) | | 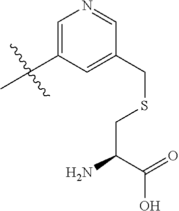 [SMILES: *c1cncc(CSC[C@H](N)C(=O)O)c1] |
| Cys(3MeBn) | | 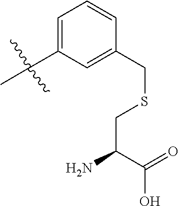 [SMILES: *c1cccc(CSC[C@H](N)C(=O)O)c1] |
| Cys(Bzl) | S-Benzylcysteine | 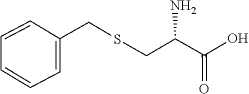 [SMILES: N[C@@H](CSCc1ccccc1)C(=O)O] |
| Cys(tMeBn (DOTA- AET)) | | 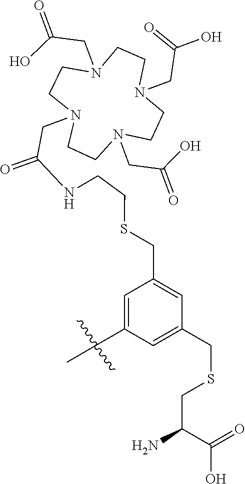 [SMILES: *c1cc(CSCCNC(=O)CN2CCN(CC(=O)O)CCN(CC(=O)O)CCN(CC(=O)O)CC2)cc(CSC[C@H](N)C(=O)O)c1] |
| Cys(tMeBn (DOTA-PP)) | | 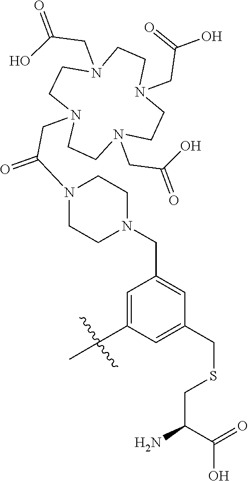 [SMILES: *c1cc(CSC[C@H](N)C(=O)O)cc(CN2CCN(C(=O)CN3CCN(CC(=O)O)CCN(CC(=O)O)CCN(CC(=O)O)CC3)CC2)c1] |
| Dab | (S)-2,4-Diaminobutyric acid | 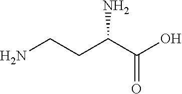 |
| Dap | (S)-2,3-Diaminopropionic acid | 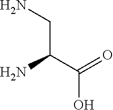 |
| Dga | Carboxymethoxy-acetic acid | 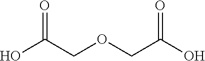 |
| DImAc | 2-{2,5-dioxo- octahydroimidazo[4,5- d]imidazolidin-1-yl}acetic acid | 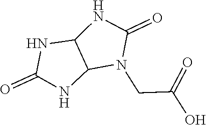 [SMILES: O=C(O)CN1C(=O)NC2NC(=O)NC21] |
| Dip | 3,3-Diphenylalanine | 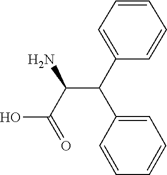 [SMILES: N[C@H](C(=O)O)C(c1ccccc1)c1ccccc1] |
| DkpAc | 2-(3,6-dioxopiperazin-2- yl)acetic acid | 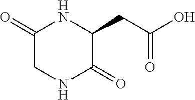 [SMILES: O=C(O)C[C@@H]1NC(=O)CNC1=O] |
| Dmo | (S)-Dimethylornithine | 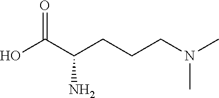 |
| dmo | (R)-Dimethylornithine | 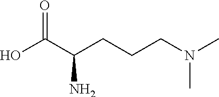 |
| DOTA | 1,4,7,10- Tetraazacyclododecane- 1,4,7,10-tetraacetic acid | 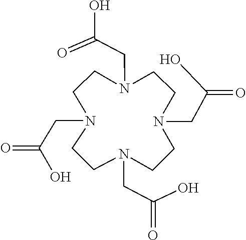 [SMILES: O=C(O)CN1CCN(CC(=O)O)CCN(CC(=O)O)CCN(CC(=O)O)CC1] |
| Eaa | 3,4-Dichlorophenylalanine | 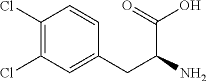 [SMILES: N[C@@H](Cc1ccc(Cl)c(Cl)c1)C(=O)O] |
| Eap | 4-(tert-Butyl)-phenylalanine | 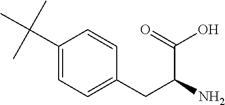 [SMILES: CC(C)(C)c1ccc(C[C@H](N)C(=O)O)cc1] |
| Eca | N1-Amino-1-cyclopentane carboxylic acid | 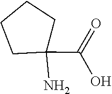 |
| Eem | S-Benzyl-cysteine-sulfone | 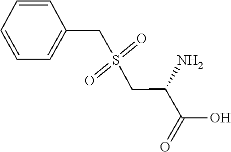 [SMILES: N[C@@H](CS(=O)(=O)Cc1ccccc1)C(=O)O] |
| Egc | 5-Methyl-DL-Tryptophan | 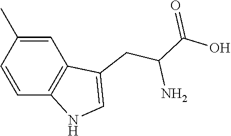 [SMILES: Cc1ccc2[nH]cc(CC(N)C(=O)O)c2c1] |
| Egm | 2,4-Dichlorophenylalanine | 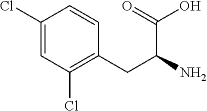 [SMILES: N[C@@H](Cc1ccc(Cl)cc1Cl)C(=O)O] |
| Egz | 1-Amino-cyclohexyl-1- carboxylic acid | 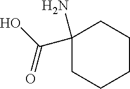 |
| EuDOTA | DOTA complexing Europium | 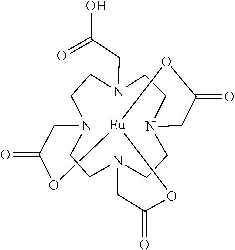 |
| FAc | fluoro acetic acid | 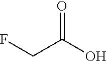 |
| FAM | 5/6-Carboxyfluorescin | 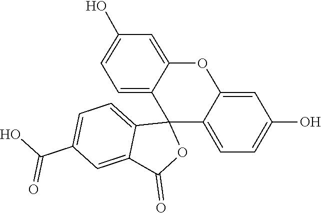 [SMILES: O=C(O)c1ccc2c(c1)C(=O)OC21c2ccc(O)cc2Oc2cc(O)ccc21] |
| FITC | Fluorescein 5/6-isothiocyanate | 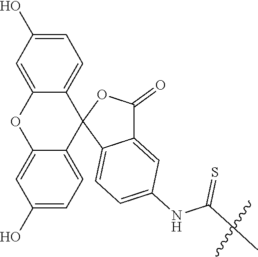 [SMILES: *C(=S)Nc1ccc2c(c1)C(=O)OC21c2ccc(O)cc2Oc2cc(O)ccc21] |
| Gab | γ-Aminobutyric acid | 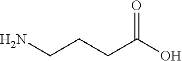 |
| GaDOTA | DOTA complexing Gallium | 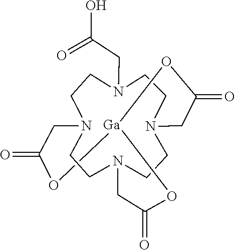 |
| Glu(AGLU) | | 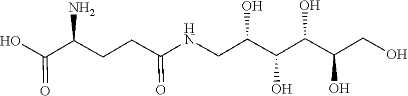 [SMILES: N[C@@H](CCC(=O)NC[C@H](O)[C@@H](O)[C@H](O)[C@H](O)CO)C(=O)O] |
| glu(AGLU) | | 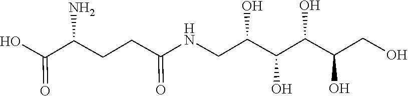 [SMILES: N[C@H](CCC(=O)NC[C@H](O)[C@@H](O)[C@H](O)[C@H](O)CO)C(=O)O] |
| Glu(Apr- DOTA) | | 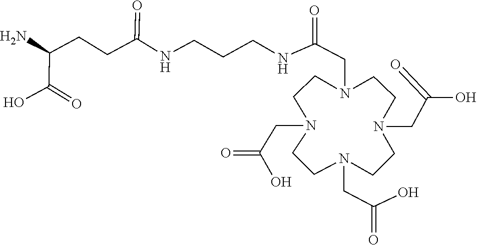 [SMILES: N[C@@H](CCC(=O)NCCCNC(=O)CN1CCN(CC(=O)O)CCN(CC(=O)O)CCN(CC(=O)O)CC1)C(=O)O] |
| Glu(Apr- O2Oc-DOTA) | | 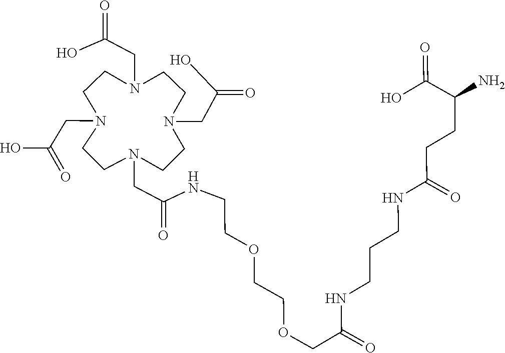 [SMILES: N[C@@H](CCC(=O)NCCCNC(=O)COCCOCCNC(=O)CN1CCN(CC(=O)O)CCN(CC(=O)O)CCN(CC(=O)O)CC1)C(=O)O] |
| Glu(Apr- O2Oc- InDOTA) | | 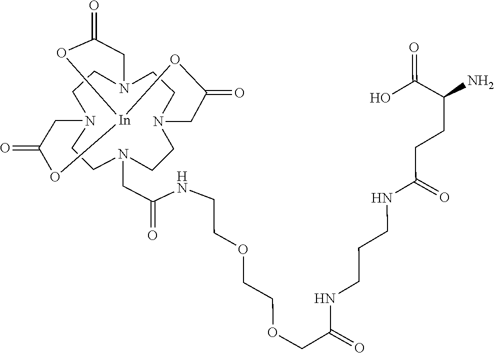 |
| Glutar | Glutaric acid | 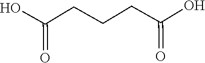 |
| H2N-Succinyl | Succinic acid amide | 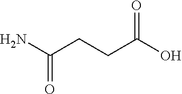 |
| Hcy | (S)-Homocysteine | 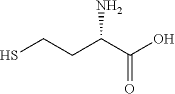 |
| Hex | Hexanoic acid | 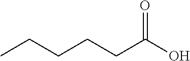 |
| HO-Succinyl | Succinic acid | 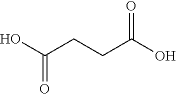 |
| Hse | (S)-Homoserine | 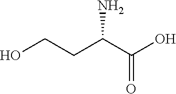 |
| Hsfu | 4-sulfamoylfuran-2-carboxylic acid | 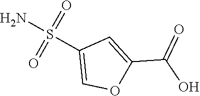 [SMILES: NS(=O)(=O)c1coc(C(=O)O)c1] |
| Hspy | 4-sulfamoyl-1H-pyrrole-2- carboxylic acid | 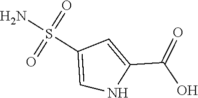 [SMILES: NS(=O)(=O)c1c[nH]c(C(=O)O)c1] |
| HYDAc | Hydantoin-5-acetic acid | 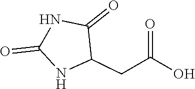 [SMILES: O=C(O)CC1NC(=O)NC1=O] |
| Hyfu | 5-[(tert- butoxy)carbamoyl]furan-3- carboxylic acid | 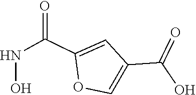 [SMILES: O=C(O)c1coc(C(=O)NO)c1] |
| HySuc | 2-(3,6-dioxo-1,2,3,6- tetrahydropyridazin-4-yl)acetic acid | 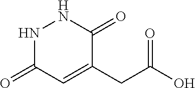 [SMILES: O=C(O)Cc1cc(=O)[nH][nH]c1=O] |
| Hyw | 5-Hydroxytryptophane | 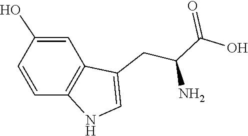 [SMILES: N[C@@H](Cc1c[nH]c2ccc(O)cc12)C(=O)O] |
| Ida | iminodiacetic acid | 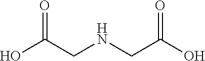 |
| Im5 | 1H-imidazole-5-carboxylic acid | 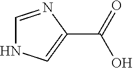 |
| InDOTA | DOTA complexing Indium | 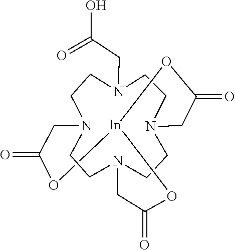 |
| iNic | Isonicotinic acid | 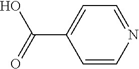 |
| Inp | Isonipecotic acid | 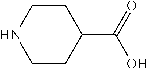 |
| Iva | Isovaleric acid | 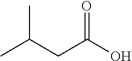 |
| LuDOTA | DOTA complexing Lutetium | 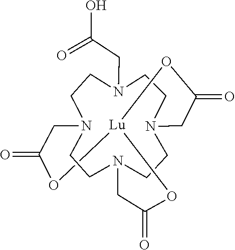 |
| Lys(DOTA) | | 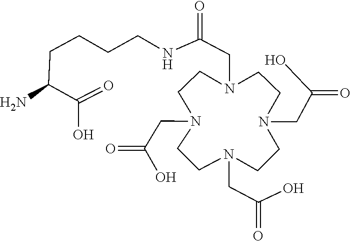 [SMILES: N[C@@H](CCCCNC(=O)CN1CCN(CC(=O)O)CCN(CC(=O)O)CCN(CC(=O)O)CC1)C(=O)O] |
| Mamb | 3-Aminomethyl-benzoic acid | 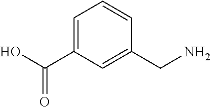 [SMILES: NCc1cccc(C(=O)O)c1] |
| MCprAc | 1-Methylcyclopropane-1- carboxylic acid | 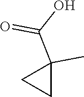 |
| MeSuc | 2-R-Methyl-succinic acid | 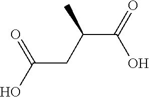 |
| mMeBz | meta-Methyl-benzoic acid | 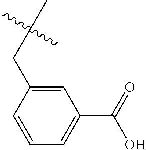 [SMILES: *Cc1cccc(C(=O)O)c1] |
| MSAc | 2-Methanesulfonyl acetic acid | 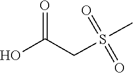 |
| Mtf | (2S)-2-Amino-3-[3- (trifluoromethyl)phenyl]propanoic acid | 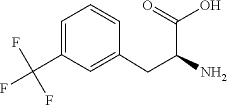 [SMILES: N[C@@H](Cc1cccc(C(F)(F)F)c1)C(=O)O] |
| N4AzPhCON H2 | 4-(3-aminoazetidin-1- yl)benzonitrile | 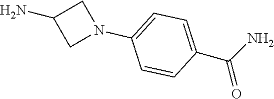 [SMILES: NC(=O)c1ccc(N2CC(N)C2)cc1] |
| N4BzlCl | (4- methanesulfonylphenyl) methanamine | 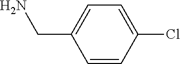 |
| N4BzlCN | 4-(aminomethyl)benzonitrile | 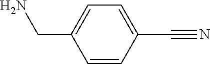 |
| N4BzlCONH2 | 4-(aminomethyl)benzamide | 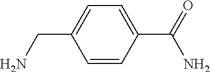 [SMILES: NCc1ccc(C(N)=O)cc1] |
| N4BzlSO2Me | (4- methanesulfonylphenyl) methanamine | 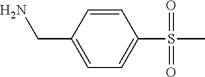 |
| N4DazPhCN | 4-(1,4-diazepan-1-yl)-3- fluorobenzonitrile hydrochloride | 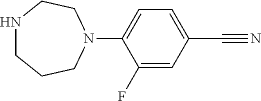 [SMILES: N#Cc1ccc(N2CCCNCC2)c(F)c1] |
| N4Inda | C-(1H-Indazol-6-yl)- methylamine | 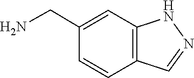 [SMILES: NCc1ccc2cn[nH]c2c1] |
| N6iQui | isoquinolin-6-ylmethanamine | 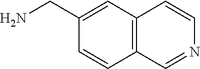 [SMILES: NCc1ccc2cnccc2c1] |
| N6MeQuion | 4-(1,4-diazepan-1-yl)-3- fluorobenzonitrile hydrochloride | 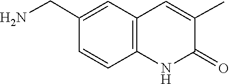 |
| NH3PhSa | 3-Aminobenzne-1-Sulfonamide | 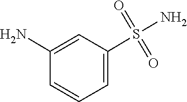 [SMILES: Nc1cccc(S(N)(=O)=O)c1] |
| NH4PhSa | 4-Aminobenzne-1-Sulfonamide | 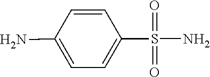 [SMILES: Nc1ccc(S(N)(=O)=O)cc1] |
| NHMe2Nph | 1-(Naphthalen-2- yl)methanamine | 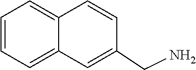 [SMILES: NCc1ccc2ccccc2c1] |
| Nic | Nicotinic acid | 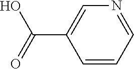 |
| NInda | 5-nitro-1H-indazole-3- carboxylic acid | 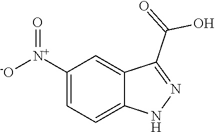 |
| Nle | (S)-Norleucine | 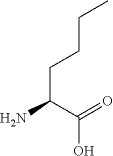 |
| Nlys | 4-Aminobutyl-glycine | 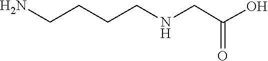 |
| Nma | (S)-N-Methyl-alanine | 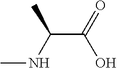 |
| nma | (R)-N-Methyl-alanine | 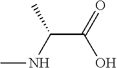 |
| Nmd | (S)-N-Methyl-aspartic acid | 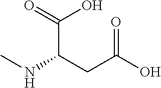 |
| Nmg | N-Methyl-glycine | 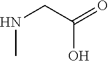 |
| Nms | (S)-N-Methyl-serine | 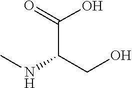 |
| Nmy | (S)-N-Methyl-tyrosine | 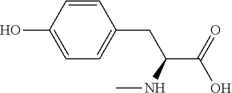 |
| NOAzOMe | [3-(methoxymethyl)-1,2-oxazol- 5-yl]methanamine | 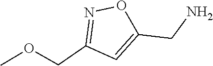 |
| Npg | Neopentyl-glycine | 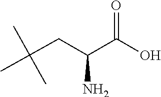 |
| O2Oc | 8-Amino-3,6-dioxaoctanoic acid | 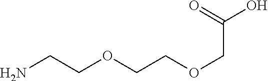 [SMILES: NCCOCCOCC(=O)O] |
| Oa5 | 1,3-Oxazole-5-carboxylic acid | 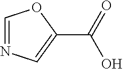 |
| OPyAc | 2(Oxopyridin-1(2H)-yl)acetic acid | 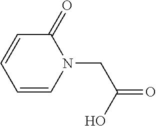 [SMILES: O=C(O)Cn1ccccc1=O] |
| Orn | (S)-Ornithine | 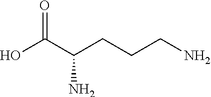 |
| orn | (R)-ornithine | 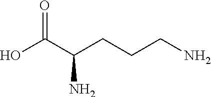 |
| Pamb | 4-Aminomethyl-benzoic acid | 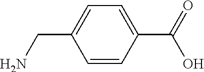 [SMILES: NCc1ccc(C(=O)O)cc1] |
| Pen | (R)-Penicillamine | 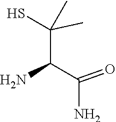 |
| PP | Piperazinyliden | 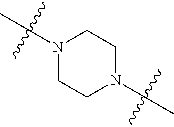 [SMILES: *N1CCN(*)CC1] |
| pGlu | L-Pyroglutamic acid | 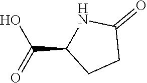 |
| Pha | Phenylacetic acic | 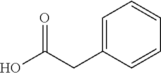 |
| Php | 3-Phenylpropionic acid | 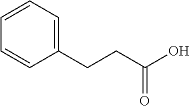 [SMILES: O=C(O)CCc1ccccc1] |
| Pif | (2S)-2-Amino-3-(4- iodophenyl)propanoic acid | 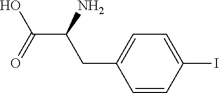 [SMILES: N[C@@H](Cc1ccc(I)cc1)C(=O)O] |
| Pip | (S)-Piperidine-2-carboxylic acid | 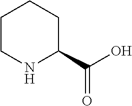 |
| pip | (R)-Piperidine-2-carboxylic acid | 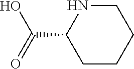 |
| PPAc | 4-Carboxymethyl piperazine | 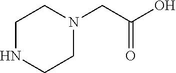 |
| PrHydr | 4-(hydroxycarbamoyl)butanoic acid | 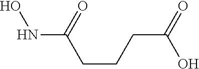 |
| Prp | Propionic acid, Propionyl | 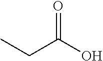 |
| Ptf | (2S)-2-amino-3-[4- (trifluoromethyl)phenyl]propanoic acid | 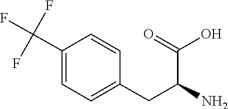 [SMILES: N[C@@H](Cc1ccc(C(F)(F)F)cc1)C(=O)O] |
| Rni | (R)-nipecotic acid | 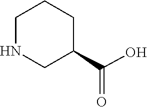 |
| SaPr | 3-Sufamoylpropanoic acid | 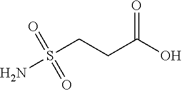 |
| Sni | (S)-nipecotic acid | 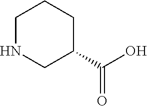 |
| Succinyl | succinic acid | 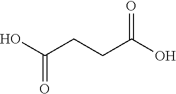 |
| Thp | 4-Amino-tetrhydropyrane-4- carboxylic acid | 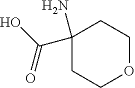 |
| Tic | (S)-1,2,3,4- Tetrahydroisoquinoline-3- carboxylic acid | 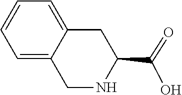 [SMILES: O=C(O)[C@@H]1Cc2ccccc2CN1] |
| Tle | (2S)-2-Amino-3,3- dimethylbutanoic acid | 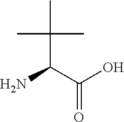 |
| tMeBn | 1,3,5-Trimethylbenzyliden | 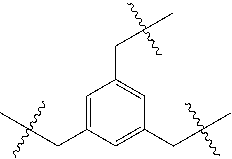 [SMILES: *Cc1cc(C*)cc(C*)c1] |
| Ttds | 1,13-Diamino-4,7,10- trioxatridecan-succinamic acid | 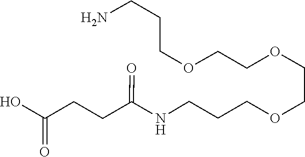 [SMILES: NCCCOCCOCCOCCCNC(=O)CCC(=O)O] |
| Tyr(Bzl) | 4-Benzyloxy-L-phenylalanine | 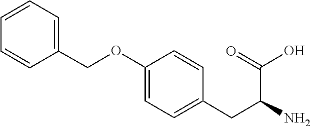 [SMILES: N[C@@H](Cc1ccc(OCc2ccccc2)cc1)C(=O)O] |
| TzPr | 3-(1H-1,2,4-triazol-3- yl)propanoic acid | 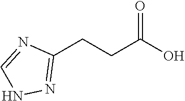 |

The amino acid sequences of the peptides provided herein are depicted in typical peptide sequence format, as would be understood by the ordinary skilled artisan. For example, the three-letter code of a natural amino acid, or the code for a non-natural amino acid or the abbreviations for additional building blocks, indicates the presence of the amino acid or building block in a specified position within the peptide sequence. The code for each amino acid or building block is connected to the code for the next and/or previous amino acid or building block in the sequence by a hyphen which (typically represents an amide linkage). If the hyphen stands before the abbreviation of the amino acid it usually symbolizes that the amino group of the amino acid is modified by a covalent bond and if the hyphen stands behind the amino acid abbreviation it usually symbolizes the modification of the former carboxyl group by a covalent bond. The remaining characteristic part of amino acids after modification by one or more covalent bonds is referred as residue of amino acid. Depending on the context the mentioning of the abbreviation of an amino acid or building block can symbolize either the full amino acid or building block or the residues of them. In connection with a use of a hyphen next to the abbreviation it is clearly specified that the residue of the amino acid or building block is addressed.

Depending on the spacing between the amino- and the carboxy group in amino acids they are classified into α-, β-, γ-, δ-, ε-, (and so forth)-amino acids, which means that these groups are typically spaced apart by 1, 2, 3, 4, and 5 atoms (typically carbon), respectively.

For amino acids, in their abbreviations the first letter indicates the stereochemistry of the C-α-atom if applicable. For example, a capital first letter indicates that the L-form of the amino acid is present in the peptide sequence, while a lower case first letter indicating that the D-form of the correspondent amino acid is present in the peptide sequence. If the abbreviation starts with a number the first letter in the abbreviation will be characteristic for the stereochemistry, if applicable. However, for enhanced clarity it is at any abbreviation an option to further clearly specify the stereochemistry of an amino acid abbreviation by adding for instance the prefix “D-”. As example “lys”, “D-Lys” or “D-lys” describe all a D-configured Lys.

For someone skilled in the art it is evident that many amino acids can be N-methylated at their amino group. These N-methyl amino acid feature can occur in combination with some other attributes like L-α- or D-α-N-methyl amino acids which are N-methylated L-α- or D-α-amino acids.

The term “α,α-dialkylamino acid” refers to amino acids which comprise independently two alkyl groups at the α-carbon atom which may in some cases form a ring-structure with each other to form a cyclic α,α-dialkylamino acid. A typical example of α,α-dialkylamino acid is 2-aminoisobutyric acid (Aib).

The term “cyclic α,α-dialkylamino acid” refers to achiral, D-, or L-α,α-dialkylamino acids wherein the two alkyl residues substituting the α-amino group combine to form a cyclic structure. The resulting cyclic structure may comprise, e.g., 4 to 7 C atoms as in 1-amino-1-cyclopentane carboxylic acid. One or more of the carbon atoms of the cyclic structure may be substituted by a heteroatom, for instance O, S, or N.

The term “aromatic amino acid” refers to amino acids which comprise an aromatic structure and this includes a heteroaromatic structure whereas the term “non-aromatic amino acid” refers to amino acids which are devoid of any aromatic structure. Preferably, the term “aromatic amino acid” refers to an amino acid selected from the group consisting of Phe, Trp, Tyr, His, Mamb, Pamb, and their derivatives, such as substituted Phe.

The term “heteroaromatic amino acid” refers to amino acids which comprise any kind of heteroaromatic structure.

An “aliphatic amino acid” is a non-aromatic amino acid which consists of only C and H atoms apart from the amino and carboxy group. Preferably, the term “aliphatic amino acid” refers to an amino acid selected from the group consisting of Gly, Ala, Val, Leu, Ile, Pro, Npg, Cha, Egz and their derivatives, more preferably from Gly, Ala, Val, Leu, Ile and Pro.

A “polar amino acid” is any kind of amino acid which comprises, apart from the amino and carboxy group, at least one functional group or atom selected from the group consisting of O, S, P, OH, and N but introduces no additional charge (at a pH ranging from about 4 to about 8) due to this functional group or atom. Preferably, the term “polar amino acid” refers to an amino acid selected from the group consisting of Asn, Gln, Ser, Thr, Cys and Tyr, more preferably from Asn, Gln, Ser, and Thr.

A “charged amino acid” is any kind of amino acid which comprises, apart from the amino and carboxy group, at least one functional group that leads to a net charge at a pH ranging from about 4 to about 8, such as COOH, phosphate, phosphonate, sulfonate, sulfate, imidazole, pyridine, guanidinium, ammonium and amino nitrogen. Preferably, the term “charged amino acid” refers to an amino acid selected from the group consisting of Asp, Glu, Lys, Arg, Orn, Dab, Dap, APac and His, more preferably from Asp, Glu, Lys and Arg.

A “neutral amino” acid is any kind of amino acid which does not have a net charge at a pH ranging from about 4 to about 8. Preferably, the term “neutral amino acid” refers to an amino acid selected from the group of aliphatic, aromatic or polar amino acids.

The expression “hydrophobic amino acids” or related terms such as “hydrophobic moieties provided by the residues of amino acids” is referring to neutral amino acids which comprise to a large extent mainly a hydrophobic moiety apart from their amino and carboxy group.

Preferably, the ratio of the sum of aliphatic, aromatic carbon and halogen atoms to heteroatoms like 0, N, and S is at least 4:1. In some embodiments, the term “hydrophobic amino acid” refers to Gly, Ala, Val, Leu, Aic, Ile, Pro, Tyr, Phe, Eaa, naphthylalanine and Trp, preferably to Ala, Val, Leu, Ile, Pro, Tyr, Phe, and Trp.

“N—(C₁-C₆)alkyl glycine” is an N-alkylated glycine wherein the alkyl rest is (C₁-C₆)alkyl which is optionally substituted, preferably with one substituent selected from the group consisting of OH, NH₂, NH, COOH, CONH₂, and S.

“S-alkylated cysteine” is a cysteine which comprises sulfur atom which is alkylated and is then part of a thioether functionality. A typical alkylating agent may be of benzylic nature.

The alkylation preferably leads to the substitution by a (C₁-C₅)alkyl-(C₅-C₁₀)aryl or (C₁-C₆)alkyl residue.

“Aza-analogue” of an aromatic amino acid is an analogue wherein one or more carbon atoms of the respective aromatic part of the amino acid are exchanged by a nitrogen atom preferably only one carbon atom is exchanged by a nitrogen atom, e.g., 7-aza-tryptophane [7Nw] is an exemplary aza-analogue of tryptophane.

If an amino acid contains more than one amino and/or carboxy group all orientations of this amino acid are in principle possible for formation of a covalent bond, but in α-amino acid the utilization of the α-amino and the α-carboxy group is preferred for the attachment to the neighbouring moieties and if other orientations are preferred they are explicitly specified.

Those skilled in the art will recognize if a stereocenter exists in the compounds disclosed herein irrespective thereof whether such stereocenter is part of an amino acid moiety or any other part or moiety of the compound of the invention. When a compound is desired as a single enantiomer or diastereomer, it may be obtained by stereospecific synthesis or by resolution of the final product or any convenient intermediate. Resolution of the final product, an intermediate, or a starting material may be affected by any suitable method known in the art. See, for example, “Stereochemistry of Organic Compounds” by E. L. Eliel, S. H. Wilen, and L. N. Mander (Wiley-Interscience, 1994).

Unless indicated to the contrary, the amino acid sequences are presented herein in N- to C-terminal direction.

It will be appreciated by a person skilled in the art that Iva, Ac, 3OHPr and 4OHPhp are building blocks comprising a carboxylic acid. They are typically incorporated into compounds of the invention by forming an amide bond with an amino group of the peptide. In preferred embodiments, they modify the N-terminus of the compounds of the inventions.

It will, for example, be appreciated by a person skilled in the art that “Ac” as abbreviation for acetic acid after forming an amide bond to its neighbor converts to “Ac-” which stands for acetyl (bound to any partner).

### Linear Peptides

A general linear peptide is typically written from the N- to C-terminal direction as shown below:

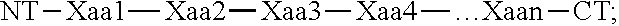

Therein
   1. Xaax is the abbreviation, descriptor or symbol for amino acids or building blocks at specific sequence position x as shown in Table 5,
   2. NT is a N-terminal group, e.g. ‘H’ (Hydrogen for a free N-terminal amino group) or an abbreviation for a specific terminating carboxylic acid like ‘Ac’ for acetic acid or other chemical group or structural formula of chemical groups linked to the N-terminal amino acid code (Xaa1) via a hyphen and
   3. CT is a C-terminal group which is typically ‘OH’ or ‘NH₂’ (as terminal carboxylic acid or amide) or an abbreviation for a specific terminating amine linked to the C-terminal amino acid code (Xaan) via a hyphen.
      Branched Peptides with Side Chains Modified by Specific Building Blocks or Peptides

A general linear, branched peptide is written from the N- to C-terminal direction as shown below:

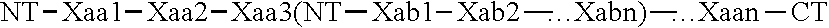

Therein the statements 1.-3. of the description of linear peptides for the specification of Xaax, NT and CT in the main chain of the branched peptide apply.

The position of a branch is specified by parentheses after a Xaax abbreviation. Branches typically occur at lysine (Lys) residues (or similar), which means that the branch is attached to side chain 8-amino function of the lysine via an amide bond.

The content of the parenthesis describes the sequence/structure of the peptide branch ‘NT-Xab1-Xab2- . . . Xabn’. Herein
1. Xabx is the abbreviation, descriptor or symbol for amino acids or building blocks at specific sequence position x of the branch as shown in Table 3,
2. NT is a N-terminal group, e.g. an abbreviation for a specific terminating carboxylic acid like ‘Ac’ for acetic acid or other chemical group or structural formula of chemical groups linked to the N-terminal amino acid code (Xab1) via a hyphen and
3. the last building block of the branch Xabn, which connects the branch with the main chain by forming an amide bond with its own carboxyl function with the side chain amino function of this lysine (or similar residue).
   Cyclic Peptides—Type I—with Direct Side Chain to Side Chain Cyclization-Connection

An exemplary general Type I cyclic peptide written from the N- to C-terminal direction is shown below:

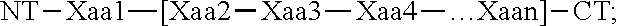

Therein the statements 1.-3. of the description of linear peptides for the specification of Xaax, NT and CT in the main chain of the cyclic peptide apply. The characteristics of the peptide cycle are specified by square brackets.
1. The opening square bracket indicates the building block at whose side chain the cycle is initiated (cycle initiation residue) and
2. the closing square bracket indicates the building block at whose side chain the cycle is terminated (cycle termination residue).

In the exemplary general cyclic peptide shown above the side chain of Xaa2 is linked directly to the side chain of Xaan.

The chemical nature of the connection between these two residues is
1. an amide bond in case that among those indicated residues one residue contains an amino function its side chain (e.g. Lys) while the other contains a carboxyl function in its side chain (e.g. Glu) or
2. a disulphide bond in case that those indicated residues/amino acids contain sulfhydryl moieties (e.g. Cys).
   Cyclic Peptides—Type II—with a Bridging Element Intramolecularly Connecting Two Side Chains by Forming a Macrocycle

An exemplary general Type II cyclic peptide with a bridging element written from the N- to C-terminal direction is shown below:

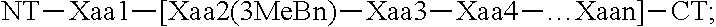

1. Therein the statements 1.-3. of the description of linear peptides for the specification of Xaax, NT and CT in the main chain of the cyclic peptide apply.
2. For the characteristics of the peptide cycle the statements 1, and 2. of the description of cyclic peptides (Type I—with direct side chain to side chain cyclization-connection) apply. In type II cycles both the cycle initiation and cycle termination residue are cysteines. Consequently, in the generic formula above Xaa2 is Cys and Xaan is Cys.
3. Furthermore the ‘3MeBn’-descriptor in parentheses right adjacent to cycle initiation residue Xaa2 indicates that a m-Xylene (3-Methylbenzylidene) unit is inserted into the peptide cycle as a bridging element. Both cysteine cycle residue side chains (Xaa2 and Xaan) are connected to the individual methyl groups of the bridging m-Xylene unit by thioether linkages.

In the exemplary general cyclic peptide with a bridging element shown above the side chain of Xaa2 is linked via 3-Methylbenzylidene-unit to the side chain of Xaan. It is obvious to the person skilled in the art that the position of the cycle initiation—as well as the cycle termination-residue can be at variable positions in the peptide sequence and are indicated in each specific sequence of the compounds of invention.

As non-limiting example, the structure of DOTA-APAc-Val-Tyr-[Cys(3MeBn)-Glu-pro-Asp-Trp-Leu-Thr-Trp-Ser-Cys]-NH₂ is depicted below.

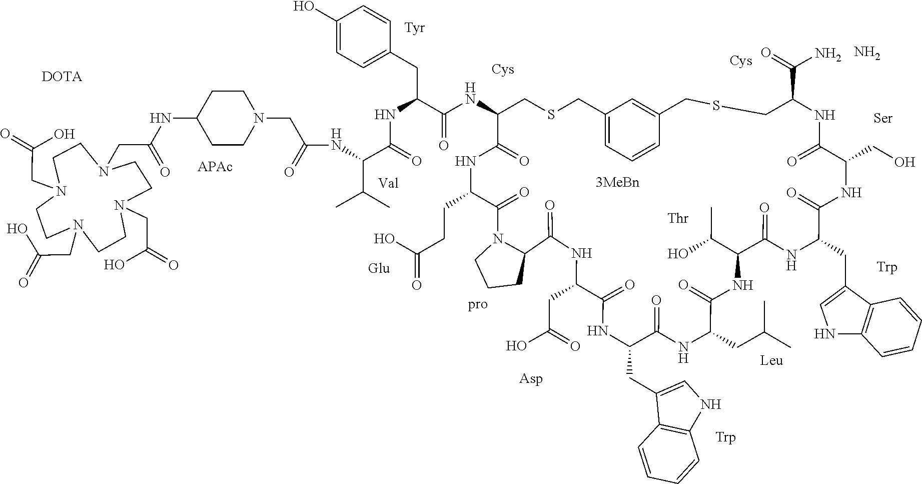

Therein
   1. DOTA and APAc correspond to NT in the general formula.
   2. Val, Tyr, Cys, Glu, pro, Asp, Trp, Leu, Thr, Trp, Ser and Cys correspond to Xaa1 to Xaa12 in the general formula.
   3. NH₂ corresponds to CT in the general formula.
   4. The opening square bracket (‘[’) adjacent to the N-terminal cysteine in the sequence indicates that at this residue the cycle is initiated (cycle initiation residue).
   5. The closing square bracket (‘]’) adjacent to the N-terminal cysteine in the sequence indicates that at this residue the cycle is terminated (cycle termination residue).
   6. 3MeBn within the parentheses adjacent to the Cys indicated as initiation residue specifies the cyclization extension element. It is further bound to the Cys indicated as cycle termination residue. The extension element is connected to said residues via thioether linkages.
      Cross Bridged Cyclic Peptides—Type III—with Both a Direct Side Chain to Side Chain Cyclization-Connection and a Cyclization with a Bridging Element Intramolecularly Connecting Two Side Chains by Forming an Additional Cross-Bridging Macrocycle

An exemplary general extended Type III cyclic peptide written from the N- to C-terminal direction is shown below:

NT-Xaa1-[Xaa2(3MeBn)-{Xaa3-Xaa4-Xaa5} . . . Xaan]-CT;
1. The statements 1.-3. of the description of linear peptides for the specification of Xaax, NT and CT in the main chain of the cyclic peptide apply.
2. The statements 2 and 3 of the description of cyclic peptides (Type II—with a bridging element intramolecularly connecting two side chains by forming a macrocycle) apply. The cycle with the bridging element is indicated by the open square bracket (‘[’) left adjacent to and the ‘3MeBn’-descriptor in parentheses right next to the cycle initiation residue, as well as the closing square bracket (‘]’) right adjacent to the cycle termination residue.
3. Furthermore the statements of the description of cyclic peptides (Type I) apply with the exceptions that
   a. an opening curly bracket (‘{’) in place of an opening square bracket indicates the cycle initiation residue and
   b. a closing curly bracket (‘}’) in place of a closing square bracket indicates the cycle termination residue.

In the exemplary general cross-bridged cyclic peptide shown above the side chain of Xaa2 is linked via 3-Methylbenzylidene-unit to the side chain of Xaan and the side chain of Xaa3 is directly linked to the side chain of Xaa5. It is obvious to the person skilled in the art that the positions of both cycle initiation—as well as both cycle termination-residues can be at variable positions in the peptide sequence and are indicated in each specific sequence of the compounds of invention.

As non-limiting example the structure of DOTA-APAc-Val-{Asp-[Cys(3MeBn)-Glu-pro-Asp-Trp-Leu-Thr-Trp-Dap}-Cys]-NH₂ is depicted below.

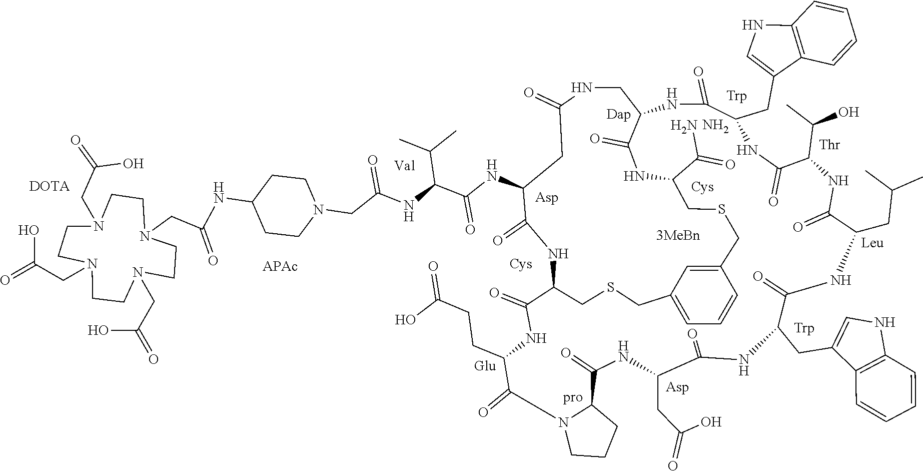

Therein
   1. DOTA and APAc correspond to NT in the general formula.
   2. Val, Asp, Cys, Glu, pro, Asp, Trp, Leu, Thr, Trp, Dap and Cys correspond to Xaa1 to Xaa12 in the general formula.
   3. NH₂ corresponds to CT in the general formula.
   4. The opening square bracket (‘[’) adjacent left to the N-terminal cysteine (Cys) in the sequence indicates that at this residue the cycle is initiated (cycle initiation residue).
   5. The closing square bracket (‘]’) adjacent right to the N-terminal cysteine (Cys) in the sequence indicates that at this residue the cycle is terminated (cycle termination residue).
   6. 3MeBn within the parentheses adjacent to the Cys indicated as initiation residue specifies the cyclization bridging element. It is further bound to the Cys indicated as cycle termination residue. The bridging element is connected to said residues via thioether linkages.
   7. The opening curly bracket (‘{’) adjacent left to the N-terminal aspartic acid (Asp) in the sequence indicates that at this residue the direct side-chain to side-chain cycle is initiated (cycle initiation residue).
   8. The closing curly bracket (‘}’) adjacent right to the diamino propionic acid (Dap) in the sequence indicates that at this residue the direct side-chain to side-chain cycle is terminated (cycle termination residue).
   9. Since the side chains of Asp and Dap are connected to each other the direct peptide cycle is a macrolactame.

In the present disclosure, where a compound of the invention is referred to by a specific code name 3BP-XYZ, such as 3BP-4452 or 3BP-4501, this code name can be interchangeably used with the code name DPI-XYZ (the two names 3BP-XYZ and DPI-XYZ thus define the same compound). Therefore, for example, a compound referred to as 3BP-4452 can also be referred to as DPI-4452, and vice versa.

The term “effector” characterizes a chemical moiety and/or element (e.g., a naturally occurring or synthetic substance) attached to the peptide for the purpose of diagnostic and/or therapeutic intervention with CAIX receptor-related diseases and/or cancer cells. In some embodiments, the term “effector” is to be understood as a moiety (e.g., chromophore, fluorophore, radiolabeled moiety, chelator comprising a chelated diagnostically active nuclide) that enables and/or facilitates the detection and/or visualization of a complementary moiety to which it is attached. For instance, the moiety can be detected and/or visualized by molecular imaging techniques known in the art such as single photon emission computed tomography (SPECT), positron emission tomography (PET), etc. In some embodiments, the term “effector” is to be understood as a pharmacologically active substance (e.g., chelator comprising a chelated therapeutically active nuclide, cytotoxic drug) which can inhibit or prevent the function of cells and/or kill cells. In some embodiments, the term “effector” is to be understood as being synonymous with other terms commonly used in the art such as “cytotoxic agent”, “toxin” or “drug” used in the field of cancer therapy.

The term “chromophore” refers to an organic or metal-organic compound which is able to absorb electromagnetic radiation in the range of from 350 nm to 1100 nm, or a subrange thereof, e.g. 350-500 nm or 500-850 nm, or 350-850 nm.

The term “phosphorophore” refers to a compound which, when excited by exposure to a particular wavelength of light, emits light at a different wavelength and lower intensity over a prolonged period of time, e.g. up to several hours.

The term “fluorophore” refers to a compound which, when excited by exposure to a particular wavelength of light, emits light at a different (higher) wavelength. Fluorophores are usually described in terms of their emission profile or “color”. For example, green fluorophores such as Cy3 or FITC generally emit at wavelengths in the range of 515-540 nm, while red fluorophores such as Cy5 or tetramethylrhodamine generally emit at wavelengths in the range of 590-690 nm. The term “fluorophore” is to be understood as encompassing, in particular, organic fluorescent dyes such as fluorescein, rhodamine, AMCA, Alexa Fluor dyes (e.g., Alexa Fluor 647), and biological fluorophores.

The term “chelator” or “chelating agent” refers to a molecule containing two or more electron donor atoms that can form coordinate bonds to a single central metal ion, e.g. to a radionuclide. Typically, chelating agents coordinate metal ions through oxygen, nitrogen, or sulfur donor atoms, or combinations thereof. After the first coordinate bond is formed, each successive donor atom that binds creates a ring containing the metal ion. A chelating agent may be bidentate, tridentate, tetradentate, etc., depending on whether it contains 2, 3, 4, or more donor atoms capable of binding to the metal ion. However, the chelating mechanism is not fully understood and depends on the chelating agent and/or radionuclide. For example, it is believed that DOTA can coordinate a radionuclide via carboxylate and amino groups (donor groups) thus forming complexes having high stability (Dai et al. Nature Com. 2018, 9, 857). The term “chelating agent” is to be understood as including the chelating agent as well as salts thereof. Chelating agents having carboxylic acid groups, e.g., DOTA, TRITA, HETA, HEXA, EDTA, DTPA etc., may, for example, be derivatized to convert one or more carboxylic acid groups to amide groups for attachment to the compound, i.e. to the reactive moiety or the linker, alternatively, for example, said compounds may be derivatized to enable attachment to the compound via one of the CH₂ groups in the chelate ring.

The term “radionuclide” as used herein refers to an atom with an unstable nucleus, which is a nucleus characterized by excess energy that is released by different types of radioactive decay. Radionuclides occur naturally or can be artificially produced. In one embodiment, references to “nuclide(s)” made in the present specification and claims are preferably to be understood as references to “radionuclide(s)”.

The expression or term “moiety derived from a drug” as used herein refers to a moiety corresponding to a native drug, which differs from the native drug only by the structural modification required for bonding to adjacent moieties, e.g. for bonding to the reactive moiety, linker or branching group comprised in the compound of the present invention. This may include covalent bonds formed by existing functional groups (available in the native drug) or covalent bonds and adjacent functional groups newly introduced for this purpose. By consequence, the drug can be used in its non-modified form (except for the replacement of e.g. a hydrogen atom by a covalent bond), or it can be chemically modified in order to incorporate one functional group allowing covalent attachment to the reactive moiety, linker, or branching group comprised in the compound of the present invention. The expression or term “moiety derived from a drug” as used herein is meant to encompass both meanings.

In an analogous manner, the term “derivative” is used to characterize moieties bonded to adjacent moieties, which moieties differ from the molecules from which they are derived only by the structural elements responsible for bonding to adjacent moieties. This may include covalent bonds formed by existing functional groups or covalent bonds and adjacent functional groups newly introduced for this purpose.

As preferably used, a “linker” refers to an element, moiety, or structure which separates or spaces apart two parts of a molecule.

A “pharmaceutically acceptable salt” of the compound of the present invention is preferably an acid salt or a base salt that is generally considered in the art to be suitable for use in contact with the tissues of human beings or animals without excessive toxicity or carcinogenicity, and preferably without irritation, allergic response, or other problem or complication. Such salts include mineral and organic acid salts of basic residues such as amines, as well as alkali or organic salts of acidic residues such as carboxylic acids. Compounds of the invention are capable of forming internal salts which are also pharmaceutically acceptable salts.

Suitable pharmaceutically acceptable salts include, but are not limited to, salts of acids such as hydrochloric, phosphoric, hydrobromic, malic, glycolic, fumaric, sulfuric, sulfamic, sulfanilic, formic, toluenesulfonic, methanesulfonic, benzene sulfonic, ethane disulfonic, 2-hydroxyethylsulfonic, nitric, benzoic, 2-acetoxybenzoic, citric, tartaric, lactic, stearic, salicylic, glutamic, ascorbic, pamoic, succinic, fumaric, maleic, propionic, hydroxymaleic, hydroiodic, phenylacetic, alkanoic such as acetic, HOOC—(CH₂)ₙ—COOH where n is any integer from 0 to 4, i.e., 0, 1, 2, 3, or 4, and the like. Similarly, pharmaceutically acceptable cations include, but are not limited to sodium, potassium, calcium, aluminum, lithium and ammonium. Those of ordinary skill in the art will recognize further pharmaceutically acceptable salts for the compounds provided herein. In general, a pharmaceutically acceptable acid or base salt can be synthesized from a parent compound that contains a basic or acidic moiety by any conventional chemical method. Briefly, such salts can be prepared by reacting the free acid or base forms of these compounds with a stoichiometric amount of the appropriate base or acid in water or in an organic solvent, or in a mixture of the two. Generally, the use of non-aqueous media, such as ether, ethyl acetate, ethanol, isopropanol or acetonitrile, is preferred.

A “pharmaceutically acceptable solvate” of the compound of the invention is preferably a solvate of the compound of the invention formed by association of one or more solvent molecules to one or more molecules of a compound of the invention. Preferably, the solvent is one which is generally considered in the art to be suitable for use in contact with the tissues of human beings or animals without excessive toxicity or carcinogenicity, and preferably without irritation, allergic response, or other problem or complication. Such solvent includes an organic solvent such as alcohols, ethers, esters and amines.

A “hydrate” of the compound of the invention is formed by association of one or more water molecules to one or more molecules of a compound of the invention. Such hydrate includes but is not limited to a hemi-hydrate, mono-hydrate, dihydrate, trihydrate and tetrahydrate.

Independent of the hydrate composition all hydrates are generally considered as pharmaceutically acceptable.

Hereinafter, in the present description of the invention and the claims, the use of the terms “containing” and “comprising” is to be understood such that additional unmentioned elements may be present in addition to the mentioned elements. However, these terms should also be understood as disclosing, as a more restricted embodiment, the term “consisting of” as well, such that no additional unmentioned elements may be present, as long as this is technically meaningful.

Unless specified otherwise or the context dictates otherwise, references to groups being “substituted” or “optionally substituted” are to be understood as references to the presence (or optional presence, as the case may be) of at least one substituent selected from F, Cl, Br, I, CN, NO₂, NH₂, NH—(C₁-C₆)alkyl, N[(C₁-C₆)alkyl]₂, —X—(C₁-C₆)alkyl, —X—(C₂-C₆)alkenyl, —X—(C₂-C₆)alkynyl, —X—(C₆-C₁₄)aryl, —X-(5-14-membered heteroalkyl with 1-3 heteroatoms selected from N, O, S), wherein X represents a single bond, —(CH₂)—, —O—, —S—, —S(O)—, —S(O)₂—, —NH—, —CO—, or any combination thereof including, for instance, —C(O)—NH—, —NH—C(O)—. The number of substituents is not particularly limited and may range from 1 to the maximum number of valences that can be saturated with substituents. It is typically 1, 2 or 3 and usually 1 or 2, most typically 1. Furthermore, e.g. in reference to Xaa7, the term “substituted” also extends to substituents NH—R^{7a} and NH—R^{7d} as defined in connection with formulae (4a) and (4b), respectively.

Unless specified otherwise, all valencies of the individual atoms of the compounds or moieties described herein are saturated. In particular, they are saturated by the indicated binding partners. If no binding partner or a too small number of binding partners is indicated, the remaining valencies of the respective atom are saturated by a corresponding number of hydrogen atoms.

Unless specified otherwise, chiral compounds and moieties may be present in the form of a pure stereoisomer or in the form of a mixture of stereoisomers, including the 50:50 racemate.

In the context of the present invention, references to specific stereoisomers are to be understood as references to compounds or moieties, wherein the designated stereoisomer is present in at least 90% enantiomeric excess (ee), more preferably at least 95% ee and most preferably 100% ee, wherein % ee is defined as (|R−S|)/(R+S)*100% with R and S representing the amount of moles of the respective enantiomers.

Unless the context dictates otherwise, and/or alternative meanings are explicitly provided herein, all terms are intended to have meanings generally accepted in the art, as reflected by IUPAC Gold Book (status of 1 Dec. 2021), or the Dictionary of Chemistry, Oxford, 8th Ed.

### 2. CHEMICAL COMPOUND, PEPTIDE, CAIX BINDING COMPOUND, CAIX BINDING PEPTIDE

The present invention relates to a chemical compound, a peptide, a Carbonic Anhydrase IX (CAIX) binding compound, and a Carbonic Anhydrase IX (CAIX) binding peptide.

The present inventors have surprisingly found that the compounds of the invention show a high affinity to Carbonic anhydrase IX. Furthermore, the present inventors have surprisingly found that the compounds of the invention show other characteristics which make them especially suitable for use in the diagnosis and therapy of diseases involving Carbonic Anhydrase IX. Such other characteristics comprise high stability in plasma and selectivity for Carbonic Anhydrase IX over other isoforms of Carbonic Anhydrase and Carbonic Anhydrase XII in particular.

Without wishing to be bound by any theory, it is considered that the cyclic peptide structure formed by amino acids Xaa3 to Xaa12 as defined herein, which includes as hydrophobic moiety an aromatic group in the bridge between the residue of amino acid Xaa3 and the residue of amino thiol Xaa12, leads to a high affinity to Carbonic anhydrase IX.

The present inventors have also found that preferred compounds of the invention comprises certain core structures or motifs.

In one embodiment (A), such core structure is formed by hydrophobic moieties provided by the residues of amino acids Xaa7, Xaa8, and Xaa10 and the aromatic group in the bridge between the residue of amino acid Xaa3 and the residue of amino thiol Xaa12, wherein Xaa1 is absent.

Without wishing to be bound by any theory, it is considered that the core structure formed by Xaa7, Xaa8 and Xaa10 confers high affinity for CAIX while the other amino acids in the cyclic peptide and the residues thereof may further enhance affinity and/or provide an appropriate and stable spacing and orientation of the mentioned fragments or groups.

In preferred modes of embodiment (A),
Xaa7 is a residue of an optionally substituted aromatic L-α-amino acid, preferably a residue of an optionally substituted Phe or a residue of an optionally substituted Trp, more preferably a residue of an optionally substituted Phe, and most preferably a residue of a substituted Phe of formula (4a) or (4b) specified herein (the term “herein” means in the present specification and/or the claims); and/or
Xaa8 is a residue of a cyclic α,α-dialkyl amino acid such as Egz, Ega, Aic, Thp, or a residue of an aliphatic L-α-amino acid, such as Leu, Npg, Nle, or Cha, more preferably a residue of a natural aliphatic L-α-amino acid, such as Leu; and/or
Xaa10 is Trp or a derivative of Trp, such as Trp substituted with a substituent selected from the group consisting of methyl, a halogen or OH, or an aza-analogue of Trp optionally substituted with methyl, a halogen or OH, preferably Trp.

In further preferred modes of embodiment (A), the above meanings of Xaa7, Xaa8 and Xaa10 are combined with at least one, e.g., 1, 2, 3, 4 or 5, preferably all, of the following preferred meanings of the remaining residues:
Xaa2 is preferably a residue of a an amino acid selected from the group consisting of a polar L-α-amino acid and a charged L-α-amino acid, more preferably a natural polar L-α-amino acid such as Gln or a natural charged amino acid such as Glu; and/or
Xaa3 is preferably a residue of an α-amino acid of formula (X) as specified herein, which has (R) configuration at the α-C-atom, more preferably L-Cys; and/or
Xaa4 is preferably a residue of an amino acid selected form the group consisting of a polar and a charged L-α-amino acid, more preferably a natural polar L-α-amino acid such as Gln or a natural charged amino acid such as Glu; and/or
Xaa5 is preferably a residue of a D-α-amino acid, Gly, Nmg, more preferably a hydrophobic D-α-amino acid, most preferably a hydrophobic D-α-amino acid such as D-pro and D-pip; and/or
Xaa6 is preferably a residue of an amino acid selected from the group consisting of a polar and a charged L-α-amino acid, more preferably a natural polar or charged (e.g. acidic) L-α-amino acid such as Asn or Asp; and/or
Xaa9 is preferably a residue of an L-α-amino acid, more preferably a polar L-α-amino acid, most preferably a polar natural L-α-amino acid such as Thr; and/or
Xaa11 is preferably a residue of a L-α-amino acid, more preferably a polar L-α-amino acid, most preferably a polar natural L-α-amino acid such as Ser; and/or
Xaa12 is preferably a residue of an amino thiol of formula (XII) as specified herein, more preferably Xaa12 is a residue of an amino thiol of formula (XIIa).

In a preferred mode of embodiment (A),
Y comprises effector E1, such as a chelator optionally comprising a chelated (radio)nuclide, wherein the effector E1 is covalently bound to Xaa2 (if Xaa1 is absent); or is Z1, wherein Z1 comprises a linker moiety L1 and an effector E1, such as chelator optionally comprising a chelated (radio)nuclide, wherein the linker moiety L1 covalently links the effector E1 to Xaa2 (if Xaa1 is absent). Preferred embodiments of the effector, chelator and optional linker L1 are described in the present specification and the claims.

In a more preferred aspect of embodiment (A), Xaa7 is an amino acid of formula (4a) or (4b) as specified herein, wherein preferably R^{7e} or R^{7g}, respectively, is (C₁-C₅)alkyl, optionally substituted with a substituent selected from the group consisting of OH, SO₂NH₂, SO₂NH—R⁷, CO(NHOH), COOH, CONH₂ and NH, more preferably —SO₂NH₂ or —COOH.

Further suitable embodiments of the above meanings of Y and Xaa2 to Xaa12 disclosed in connection with embodiment (A) are described in the present specification and claims.

In line with embodiment (Ab), compounds of embodiment (A) are modified such as to conform to bicyclic peptide structure (1b).

Compounds of embodiment (Ab) have the same preferred meanings of Y, Xaa2, Xaa3, Xaa4, Xaa5, Xaa6, Xaa7, Xaa8, Xaa9, Xaa10 and Xaa12 as specified above. As to Xaa2 and Xaa11 the following applies:
Xaa2 is a residue of an L-α-amino acid comprising, in addition to an amino group and a carboxy group attached to an α-C atom, a functional group FG1 forming a covalent linkage B1 with a functional group FG2 of Xaa11, wherein Xaa11 is a residue of an L-α-amino acid comprising, in addition to an amino group and a carboxy group attached to an α-C atom, the functional group FG2. Functional groups FG1 and FG2 can be selected from preferred embodiments described herein. FG1 is, e.g., a carboxy group as in Glu, and FG2 is, e.g., an amino group as in (S)-2,3-diaminopropionic acid [dap].

Alternatively, the 2nd cycle can be formed in embodiment (Ab), and bicyclic peptide structure (1b), with Xaa2 being Asp and Xaa11 being Dap, Xaa2 being Dap and Xaa11 being Asp, or Xaa 2 being Dap and Xaa11 being Glu.

Further suitable embodiments of the above meanings of Y and Xaa2 to Xaa12 disclosed in connection with embodiment (Ab) are described in the present specification and claims.

In one embodiment (B) of the compounds of the invention, such core structure is formed by hydrophobic moieties provided by the residues of amino acids Xaa7, Xaa8, and Xaa10 and the aromatic group in the bridge between the residue of amino acid Xaa3 and the residue of amino thiol Xaa12, wherein the compounds of embodiment (B) when compared to the compounds of embodiment (A) additionally comprise the residue of amino acid Xaa1.

In preferred modes of embodiment (B),
Xaa7 is a residue of an optionally substituted aromatic L-α-amino acid, preferably a residue of an optionally substituted Phe or a residue of an optionally substituted Trp, more preferably a residue of a substituted Phe, and most preferably a residue of a substituted Phe of formula (4a) or (4b) specified herein (the term “herein” means in the present specification and/or the claims); and/or
Xaa8 is a residue of a cyclic α,α-dialkyl amino acid such as Egz, Ega, Aic, Thp, or a residue of an aliphatic L-α-amino acid, such as Leu, Npg, Nle, or Cha, more preferably a residue of a natural aliphatic L-α-amino acid, such as Leu; and/or
Xaa10 is Trp or a derivative of Trp, such as Trp substituted with a substituent selected from the group consisting of methyl, a halogen or OH, or an aza-analogue of Trp optionally substituted with methyl, a halogen or OH, preferably Trp.

In further preferred modes of embodiment (B), the above meanings of Xaa7, Xaa8 and Xaa10 are combined with at least one, e.g., 1, 2, 3, 4 or 5, preferably all, of the following preferred meanings of the remaining residues:
Xaa1 is selected from the group consisting of Val, Ile, Tle, Thr and Ser, preferably selected from the group consisting of Val, Ile and Ser; and/or
Xaa2 is preferably a residue of an amino acid selected from the group consisting of a polar, an aromatic and a charged L-α-amino acid, preferably a natural polar L-α-amino acid such as Gln or Ser, natural aromatic L-α-amino acid such as Tyr or Phe or a natural charged amino acid such as Glu or Arg; and/or
Xaa3 is preferably a residue of an α-amino acid of formula (X) as specified herein, which has (R) configuration at the α-C-atom, preferably L-Cys; and/or
Xaa4 is preferably a residue of an amino acid selected from the group consisting of a polar and a charged L-α-amino acid, preferably a natural polar L-α-amino acid such as Gln or a natural charged amino acid such as Glu; and/or
Xaa5 is preferably a residue of a D-α-amino acid, Gly, Nmg, preferably a hydrophobic D-α-amino acid, more preferably a hydrophobic D-α-amino acid such as D-pro and D-pip; and/or
Xaa6 is preferably a residue of an amino acid selected from the group consisting of a polar and a charged L-α-amino acid, more preferably a natural polar or charged (e.g. acidic) L-α-amino acid such as Asn or Asp; and/or
Xaa9 is preferably a residue of an L-α-amino acid, more preferably a polar L-α-amino acid, most preferably a polar natural L-α-amino acid such as Thr; and/or
Xaa11 is preferably a residue of a L-α-amino acid, more preferably a polar L-α-amino acid, most preferably a polar natural L-α-amino acid such as Ser; and/or
Xaa12 is preferably a residue of an amino thiol of formula (XII) as specified herein, more preferably Xaa12 is a residue of an amino thiol of formula (XIIa).

In a preferred mode of embodiment (B),
Y comprises effector E1, such as a chelator optionally comprising a chelated (radio)nuclide, wherein the effector E1 is covalently bound to Xaa2 (if Xaa1 is absent); or is Z1, wherein Z1 comprises a linker moiety L1 and an effector E1, such as chelator optionally comprising a chelated (radio)nuclide, wherein the linker moiety L1 covalently links the effector E1 to Xaa2 (if Xaa1 is absent). Preferred embodiments of the effector, chelator and optional linker L1 are described in the present specification and the claims.

In a more preferred aspect of embodiment (B), Xaa7 is an amino acid of formula (4a) or (4b) as specified herein, wherein preferably R^{7e} or R^{7g}, respectively, is (C₁-C₅)alkyl, optionally substituted with a substituent selected from the group consisting of OH, SO₂NH₂, SO₂NH—R⁷, CO(NHOH), COOH, CONH₂ and NH, more preferably —SO₂NH₂ or —COOH.

Further suitable embodiments of the above meanings of Y and Xaa1 to Xaa12 disclosed in connection with embodiment (B) are described in the present specification and claims.

In line with embodiment (Bb), compounds of embodiment (B) are modified such as to conform to bicyclic peptide structure (1b).

Compounds of embodiment (Bb) have the same preferred meanings of Y, Xaa1, Xaa3, Xaa4, Xaa5, Xaa6, Xaa7, Xaa8, Xaa9, Xaa10 and Xaa12 as specified above. As to Xaa2 and Xaa11 the following applies:
Xaa2 is a residue of an L-α-amino acid comprising, in addition to an amino group and a carboxy group attached to an α-C atom, a functional group FG1 forming a covalent linkage B1 with a functional group FG2 of Xaa11, wherein Xaa11 is a residue of an L-α-amino acid comprising, in addition to an amino group and a carboxy group attached to an α-C atom, the functional group FG2. Functional groups FG1 and FG2 can be selected from preferred embodiments described herein. FG1 is, e.g., a carboxy group as in Glu, and FG2 is, e.g., an amino group as in (S)-2,3-diaminopropionic acid [dap].

Alternatively, the 2nd cycle can be formed in embodiment (Bb), and bicyclic peptide structure (1b), with Xaa2 being Asp and Xaa11 being Dap, Xaa2 being Dap and Xaa11 being Asp, Xaa2 being Dap and Xaa11 being Glu, Xaa2 being Glu and Xaa11 being Dap, or Xaa2 being Cys and Xaa11 being Cys.

Further suitable embodiments of the above meanings of Y and Xaa1 to Xaa12 disclosed in connection with embodiment (Bb) are described in the present specification and claims.

Further embodiments of the compound (peptide) of the present invention, as well as the broadest meanings used in connection with Y and Xaa1 to Xaa12 are explained in more detail below.

The present invention relates to a compound comprising a peptide, or to a peptide represented by the following formula (1a):

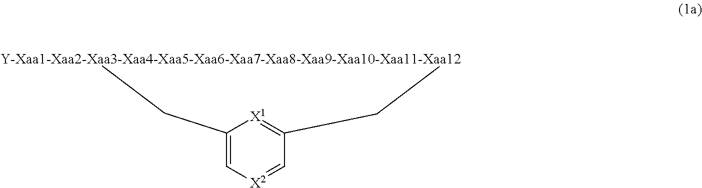

In formula (1a), Y is a moiety selected from:
(i) an N-terminal modification group A selected from the group consisting of R^{0a}—SO2-, R^{0a}—CO—, R^{0a}—NH—CO—, wherein R^{0a} is selected from the group consisting of (C₁-C₁₀)alkyl, (C₅-C₁₀)aryl, and (C₁-C₈)alkyl-(C₅-C₁₀)aryl;
(ii) a moiety comprising (or consisting of) an effector E1, wherein the effector E1 is covalently bound to Xaa1 if Xaa1 is present, or to Xaa2 if Xaa1 is absent and Xaa2 is present, or to Xaa3 if both Xaa1 and Xaa2 are absent; and
(iii) a group Z1, wherein Z1 comprises a linker moiety L1 and an effector E1, wherein the linker moiety L1 covalently links the effector E1 to Xaa1 if Xaa1 is present, or to Xaa2 if Xaa1 is absent and Xaa2 is present, or to Xaa3 if both Xaa1 and Xaa2 are absent.

In some embodiments, Y is (i) an N-terminal modification group A selected from the group consisting of 3-methyl butanoyl [Iva], Acetyl [Ac], hexanoyl [Hex], benzoyl [Bz], phenylacetyl [Pha], and propionyl [Prp]. Preferably, Y is Ac.

In some embodiments, Y is (ii) a moiety comprising (or consisting of) an effector E1, wherein the effector is selected from the group consisting of:
(α) a moiety derived from a chromophore, which is preferably selected from (a1) a phosphorophore and (a2) a fluorophore, such as fluorescein or rhodamine; and
(β) a chelator optionally comprising a chelated nuclide; and
(γ) a moiety derived from a drug, preferably from a cytotoxic drug.

In some embodiments, Y is (iii) a group Z1, wherein Z1 comprises a linker moiety L1 and an effector E1, wherein the linker moiety L1 provides (a) a carboxy group forming an amide bond with an α-amino group provided by Xaa1 if Xaa1 is present, or with an α-amino group provided by Xaa2 if Xaa1 is absent and Xaa2 is present, or with an α-amino group provided by Xaa3 if both Xaa1 and Xaa2 are absent, and (b) an amino group forming a covalent bond to the effector.

In one embodiment, the linker moiety L1 (but the following description applies also to linker moieties L3, L4 and L6) is a group comprising from 1 to 10 amino acids which is optionally cleavable, and/or the effector is as defined above. In particular, the linker may be an amino acid or a peptide consisting of up to 10 amino acids, which are independently selected from the group comprising natural amino acids, non-natural amino acids, α-amino acids and amino acids where the amino and the carboxylic group are spaced further apart such as β-amino acids, γ-amino acids, δ-amino acids, ε-amino acids, and ω-amino acids.

The linker can also be one which allows release of the effector, e.g., the conjugated drug. Preferably, the effector (e.g., drug) is released while the compound of the invention is bound to the tumor cell or resides within the tumor or in the close proximity of the tumor, e.g., in the tumor environment.

The effector (e.g., drug) may be released enzymatically, proteolytically (preferably by tumor specific proteases), by means of other enzymes (preferably tumor specific proteases), due to half-life of the conjugation (chemical or biological instability), by pH shift in the tumor environment, a tumor metabolite, a protein, a carbohydrate, a lipid or a nucleic acid present in the tumor, a co-administered agent, an external treatment or an endoscopic treatment, electromagnetic radiation (Gamma, X-ray, ultraviolet, visible, infrared, microwave radio), ultrasound, magnetic field, temperature (heat and/or cold) or physical treatment.

In an embodiment, the linker is cleavable under intracellular conditions, such that the cleavage of the linker releases the effector (e.g., drug) from the compound of the invention in the intracellular environment. In some embodiments, the linker is cleaved by a cleavable agent that is present in the intracellular environment (e.g. within a lysosome or endosome or caveola). The linker can be, e.g. a peptidyl linker that is cleaved by an intracellular peptidase or protease enzyme, including but not limited to, a lysosomal or endosomal protease. In some embodiments, the peptidyl linker is at least two amino acids long or at least three amino acids long. Cleaving agents can include cathepsins B and D and plasmin, all of which are known to hydrolyze dipeptide drug derivatives resulting in the release of effector (e.g., active drug) inside the target cells (see e.g. Dubowchik and Walker, Pharm. Therapeutics, 1999, 83, 67-123). In a specific embodiment, the peptidyl linker cleavable by an intracellular protease is a Val-Cit (valine-citrulline) linker or a Phe-Lys (phenylalanine-lysine) linker (see e.g. U.S. Pat. No. 6,214,345, which describes the synthesis of doxorubicin with the Val-Cit linker and different examples of Phe-Lys linkers). Examples of the structures of a Val-Cit and a Phe-Lys linker include but are not limited to MC-vc-PAB, MC-vc-GABA, MC-Phe-Lys-PAB or MC-Phe-Lys-GABA, wherein MC is an abbreviation for maleimido caproyl, vc is an abbreviation for Val-Cit, PAB is an abbreviation for p-aminobenzylcarbamate and GABA is an abbreviation for γ-aminobutyric acid.

An advantage of using intracellular proteolytic release of the therapeutic agent is that the agent is typically attenuated when conjugated and the serum stabilities of the conjugates are typically high. In yet another embodiment, the linker unit is not cleavable, and the drug is released by NTR1 tracer unit degradation (see US 2005/0238649). Typically, such a linker is not substantially sensitive to the extracellular environment. As used herein, “not substantially sensitive to the extracellular environment” in the context of a linker means that no more than 20%, typically no more than about 15%, more typically no more than about 10%, and even more typically no more than about 5%, no more than about 3%, or no more than about 1% of the linkers, in a sample of NTR1 tracer drug conjugate compound, are cleaved when the NTR1 tracer drug conjugate compound presents in an extracellular environment (e.g. plasma).

Whether a linker is not substantially sensitive to the extracellular environment can be determined for example by incubating the NTR1 tracer drug conjugate compound with plasma for a predetermined time period (e.g. 2, 4, 8, 16 or 24 hours) and then quantitating the amount of free drug present in the plasma.

Enzymatically cleavable sequences as shown below:
a) Dipeptides: -Phe-Lys-, -Ala-Lys-, -Val-Lys-, -Val-Cit-, -Phe-Cit-, -Ile-Cit-, -Leu-Cit-, -Trp-Cit-, -Phe-Ala-, and -Phe-Arg-; and
b) Tripeptides: -Phe-Phe-Lys-, -Val-Phe-Lys-, and -Gly-Phe-Lys-; and
c) Tetrapeptides: -Gly-Phe-Leu-Gly, and -Ala-Leu-Ala-Leu-.

The linker moiety may be optimized with regard to its sensitivity and selectivity for enzymatic cleavage by particular enzymes, for example, a tumor-associated protease. In one embodiment, the linker is one which is cleaved by cathepsin B, C or D, or by a plasmin protease.

In one embodiment, the linker is a dipeptide, tripeptide or pentapeptide. In a further embodiment, a preferred linker moiety comprises a Gly residue at the C-terminal end. In another embodiment, the linker comprises a Gly-Gly Dipeptide at the C-terminal end. In yet another embodiment, the linker comprises a C-terminal dipeptide unit capable of acting as a highly specific substrate for the exopeptidase activity of Cat B (exo-Cat B). Examples of exo-Cat B-cleavable linkers systems are described in WO 2019/096867 A1. In particular, the linker can comprise a C-terminal dipeptide unit (“Axx-Ayy” or “Ayy-Axx”) as defined in claim 1, 2 or 3 of WO 2019/096867 A1.

In this context “self-immolative” linkers are another valuable tool. The main function of these type of linker is to release the effector unit after selective trigger activation in its preferably unmodified or at least effective form via a spontaneous chemical breakdown. An often-used concept to utilize a protease cleavage as described above as initial trigger and combine this with a self-immolative linker of the para-amino-benzyl type (PAB) bound as carbamate or carbonate to the drug. A representative example of this type of combination is -Val-Cit-PAB-OC-tubulysin/cryptophycin/paclitaxene/SN-38.

In one embodiment, the linker moiety L1 is selected from the group consisting of X11 and X11-X12, wherein X11 and X12 are each and individually a residue of an amino acid, wherein if the linker moiety L1 is X11, a carboxy group is provided by X11 and if the linker moiety L1 is X11-X12, a carboxy group is provided by X12, wherein the carboxy group of L1 forms an amide bond with an α-amino group provided by Xaa1 if Xaa1 is present, or with an α-amino group provided by Xaa2 if Xaa1 is absent and Xaa2 is present, or with an α-amino group provided by Xaa3 if both Xaa1 and Xaa2 are absent and X11 provides an amino group which is forming a covalent bond to the effector.

Preferably, X11 and X12 are each and individually a residue of an amino acid selected from the group consisting of 1,13-diamino-4,7,10-trioxatridecan-succinamic acid [Ttds], 8-amino-3,6-dioxaoctanoic acid [O2Oc], 2-(4-(amino)piperidin-1-yl)acetic acid [APac], 4-Carboxymethyl piperazine [PPac], 4-trans-aminomethylcyclohexane carboxylic acid [4Amc], and an amino acid according to any one of the following formulae (32)-(34):

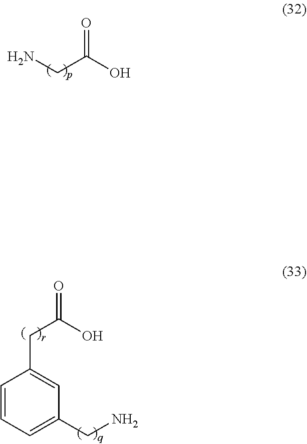

and the ortho- and para-substituted isomers thereof, and

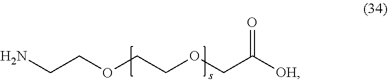

wherein
p is 2, 3, 4, 5, 6, 7, 8, 9, or 10,
q is 0, 1, 2, 3, or 4,
r is 0, 1, 2, 3, or 4,
s is 0, 1, 2, 3, 4, 5, 6, 7, 8, 9, 10, 11 or 12,
and the amino acid of formulae (32) and (33) is optionally substituted.

The amino acid of formulae (32) and (33) may be substituted with R^{X11}—CO—NH— at an α-carbon atom which is covalently bound to the COOH-group in formulae (32) and (33), wherein R^{X11} is selected from the group consisting of (C₁-C₁₀)alkyl, (C₅-C₁₀)aryl, and (C₁-C₅)alkyl-(C₅-C₁₀)aryl. Preferably, R^{X11} is methyl.

More preferably, X11 and X12 are each and individually a residue of an amino acid selected from the group consisting of 1,13-diamino-4,7,10-trioxatridecan-succinamic acid [Ttds], 8-amino-3,6-dioxaoctanoic acid [O2Oc], 2-(4-(amino)piperidin-1-yl)acetic acid [APac], 4-Carboxymethyl piperazine [PPac], 4-trans-aminomethylcyclohexane carboxylic acid [4Amc]β-Alanine [Bal], γ-Aminobutyric acid [Gab], 5-amino pentanoic acid [Ava], 6-aminohexanoic acid [Ahx], 3-aminomethyl-benzoic acid [Mamb], 4-aminomethyl-benzoic acid [Pamb] and an ε-amino acid of formula (35)

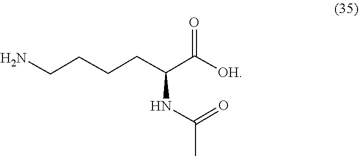

Xaa1 is either present or absent, and if present is a residue of an aliphatic or polar L-amino acid. If Xaa1 represents an aliphatic L-amino acid, the same is preferably an aliphatic L-α-amino acid, which can be selected from natural or non-natural aliphatic L-α-amino acids.

If Xaa1 represents a polar L-amino acid, the polar L-amino acid is preferably a polar L-α-amino acid, which can be selected from natural polar L-α-amino acids or non-natural polar L-α-amino acids.

In preferred embodiments, Xaa1 is selected from the group consisting of Val, Ile, (2S)-2-amino-3,3-dimethylbutanoic acid [Tle], Ser and Thr. In other preferred embodiments, Xaa1 is absent.

Xaa2 is either present or absent, wherein if Xaa2 is absent, Xaa1 is also absent and, if Xaa2 is present, (i) Xaa2 is a residue of an L-α-amino acid which is optionally N-methylated at the α-nitrogen atom, or, (ii) Xaa2 is a residue of an L-α-amino acid comprising, in addition to an amino group and a carboxy group attached to an α-C atom, a functional group FG1 forming a covalent linkage B1 with a functional group FG2 of Xaa11, wherein Xaa11 is a residue of an L-α-amino acid comprising, in addition to an amino group and a carboxy group attached to an α-C atom, the functional group FG2, wherein a bicyclic peptide of formula (1b) is formed:

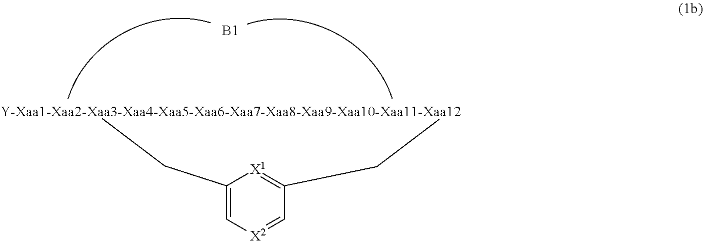

If (i) Xaa2 is a residue of an L-α-amino acid which isoptionally N-methylated at the α nitrogen atom, the same can be selected from natural or non-natural α-amino acids. According to this embodiment, Xaa2 is preferably a residue of an optionally N-methylated L-α-amino acid selected from the group consisting of an aromatic amino acid, a polar amino acid and a charged amino acid. It is further preferred that (i) Xaa2 represents a polar, optionally N-methylated L-α-amino acid, which can be selected from natural polar L-α-amino acids (e.g. Gln or Glu) or non-natural polar L-α-amino acids.

In preferred embodiments, (i) Xaa2 is a residue of an L-α-amino acid selected from the group consisting of Tyr, (S)-N-methyl-tyrosine [Nmy], Phe, Gln, Arg, (S)-dimethylornithine [Dmo], Ser, Thr, Asp, Glu and Glu(AGLU). In more preferred embodiments, (i) Xaa2 is a residue of an L-α-amino acid selected from the group consisting of Tyr, (S)-N-methyl-tyrosine [Nmy], Gln, Arg, (S)-dimethylornithine [Dmo] and Ser. Most preferably, (i) Xaa2 is Gln. According to these embodiments, Xaa1 is preferably absent.

If (ii) Xaa2 is a residue of an L-α-amino acid comprising, in addition to an amino group and a carboxy group attached to an α-C atom, a functional group FG1 forming a covalent linkage B1 with a functional group FG2 of Xaa11, the covalent linkage B1 is preferably selected from the group consisting of an amide linkage, a disulfide linkage, a thioether linkage, a thiourea linkage, a triazole linkage, a carbamate linkage, an amine linkage, a sulfonamide linkage, an ester linkage, a thioester linkage, an ether linkage, a urea linkage and a hydrocarbon linkage. More preferably, the covalent linkage B1 is selected from the group consisting of an amide linkage or a disulfide linkage. Most preferably, the covalent linkage B1 is an amide linkage.

In some embodiments, the functional group FG1 of Xaa2 forming the covalent linkage B1 with the functional group FG2 of Xaa11 is selected from the group consisting of NH₂, NH—, COOH, activated carboxylic acid, chloro, bromo, iodo, SH, OH, SOOH, activated sulfonic acid, sulfonic acid ester, Michael acceptors, isocyanate, isothiocyanate, azide, alkene, and alkyne.

In preferred embodiments, (ii) Xaa2 is a residue of an L-α-amino acid selected from the group consisting of (S)-2,3-diaminopropionic acid [Dap], (S)-2,4-diaminobutyric acid [Dab], (S)-ornithine [Orn], Lys, Cys, (S)-homocysteine [Hcy], (R)-Penicillamine [Pen], Asp and Glu. More preferably, (ii) Xaa2 is a residue of Glu.

Furthermore, the functional group FG2 of Xaa11 forming the covalent linkage B1 with the functional group FG1 of Xaa2 is preferably selected from the group consisting of NH₂, NH—, COOH, activated carboxylic acid, chloro, bromo, iodo, SH, OH, SOOH, activated sulfonic acid, sulfonic acid ester, Michael acceptors, isocyanate, isothiocyanate, azide, alkene and alkyne. In preferred embodiments, Xaa11 (which forms the covalent linkage B1 with Xaa2) is a residue of an L-α-amino acid selected from the group consisting of (S)-2,3-diaminopropionic acid [Dap], (S)-2,4-diaminobutyric acid [Dab], (S)-ornithine [Orn], Lys, Cys, (S)-homocysteine [Hcy], (R)-Penicillamine [Pen], Asp and Glu. Most preferably, Xaa11 is a residue of (S)-2,3-diaminopropionic acid [Dap]. According to these embodiments, it is preferred that Xaa1 is absent and Xaa2 is Glu.

Xaa3 is a residue of an α-amino acid, preferably of an L-α-amino acid, of formula (X):

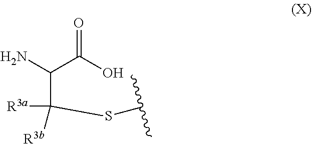

In formula (X), R^{3a} and R^{3b} are each and independently selected from the group consisting of H and CH₃. In preferred embodiments, both R^{3a} and R^{3b} are H. Most preferably, Xaa3 is a residue of (L)-Cys.

Xaa4 is a residue of an L-α-amino acid which is optionally N-methylated at the α-nitrogen atom. In preferred embodiments, Xaa4 is a residue of an L-α-amino acid selected from the group consisting of an aliphatic amino acid, a polar amino acid and a charged amino acid. In more preferred embodiments, Xaa4 is a residue of an L-α-amino acid selected from the group consisting of Ala, Ser, (S)-homoserine [Hse], (S)-N-methyl-serine [Nms], Gln, Asn, Glu, Asp, Dmo and Glu(AGLU). In even more preferred embodiments, Xaa4 is a residue of an L-α-amino acid selected from the group consisting of Ala, Ser, Glu, Gln and (S)-homoserine [Hse]. Most preferably, Xaa4 is a residue of Glu.

Xaa5 is a residue of an amino acid which is optionally bound to a moiety Z3, wherein Xaa5 is a residue of an amino acid selected from the group consisting of N—(C₁-C₆)alkyl glycine, Gly, a D-α-amino acid, and an α,α-dialkylamino acid. It is particularly preferred that Z3 is absent from (not bound to) Xaa5.

If Xaa5 comprises a moiety Z3, Z3 is (i) an effector E3, or (ii) a moiety comprising an effector E3 and a linker moiety L3, wherein the effector E3 is preferably selected from the group consisting of:
(α) a moiety derived from a chromophore, which is preferably selected from (α1) a phosphorophore and (α2) a fluorophore, such as fluorescein or rhodamine; and
(β) a chelator optionally comprising a chelated nuclide; and
(γ) a moiety derived from a drug, preferably from a cytotoxic drug.

In some embodiments Xaa5 is a residue of an amino acid wherein Z3 is absent. In this case, Xaa5 is preferably a residue of an amino acid selected from the group consisting of Gly, N-methyl-glycine [Nmg], D-ala, D-pro, (R)-piperidine-2-carboxylic acid [D-pip], (R)-azetidine-2-carboxylic acid [D-aze], (R)-N-methyl-alanine [Nma], and 2-amino-isobutyric acid [Aib], more preferably a residue of D-pro.

In some embodiments, Xaa5 is a residue of an amino acid bound to a moiety Z3, wherein Z3 (i) is an effector E3, or (ii) a moiety comprising an effector E3 and a linker moiety L3. In this case, Xaa5 is preferably a residue of an amino acid selected from the group consisting of N—(C₁-C₄)alkyl glycine, a non-aromatic D-α-amino acid, a non-aromatic N-Methyl-D-α-amino acid, a cyclic D-α-amino acid, and an α,α-dialkylamino acid, which comprises at least one functional group forming a covalent linkage with the effector E3 or the linker moiety L3. More preferably, Xaa5 is a residue of an amino acid selected from the group consisting of 4-aminobutyl-glycine [Nlys], D-lys, (R)-ornithine [D-orn], (R)-2,4-diaminobutyric acid [D-dab], and (R)-2,3-diaminopropionic acid [D-dap], and the effector E3 or linker moiety L3 is covalently attached to an N atom different from the α-nitrogen atom of any one of Nlys, D-lys, D-orn, D-dab, and D-dap.

In preferred embodiments, the bond linking the effector E3 or linker moiety L3 to the N atom different from the α-nitrogen atom is an amide bond. The linker moiety L3 may provide (a) a carboxy group forming an amide bond with the N atom different from the α-nitrogen atom of any one of 4-aminobutyl-glycine [Nlys], D-lys, (R)-ornithine [D-orn], (R)-2,4-diaminobutyric acid [D-dab], and (R)-2,3-diaminopropionic acid [D-dap], and (b) an amino group forming a covalent bond to the effector E3.

The linker moiety L3, if present, may be selected from the group consisting of X31 and X31-X32, wherein X31 and X32 are each and individually a residue of an amino acid, wherein if the linker moiety L3 is X31, a carboxy group is provided by X31 and if the linker moiety L3 is X31-X32, a carboxy group is provided by X32, wherein the carboxy group of L3 forms an amide bond with an N atom different from the α-nitrogen atom of any one of 4-aminobutyl-glycine [Nlys], D-lys, (R)-ornithine [D-orn], (R)-2,4-diaminobutyric acid [D-dab], and (R)-2,3-diaminopropionic acid [D-dap], and X3 provides an amino group which is forming a covalent bond to the effector E3. Preferably, X31 and X32 are each and individually a residue of an amino acid selected from the group consisting of 1,13-diamino-4,7,10-trioxatridecan-succinamic acid [Ttds], 8-amino-3,6-dioxaoctanoic acid [O2Oc], 2-(4-(amino)piperidin-1-yl)acetic acid [APac], 4-Carboxymethyl piperazine [PPac], 4-trans-aminomethylcyclohexane carboxylic acid [4Amc] and an amino acid according to any one of formulae (32)-(34):

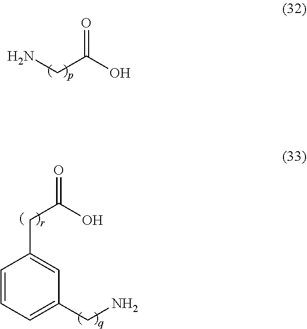

and the ortho-para-substituted isomers thereof, and

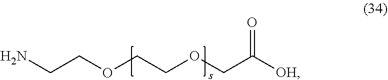

wherein
p is 2, 3, 4, 5, 6, 7, 8, 9, or 10,
q is 0, 1, 2, 3, or 4,
r is 0, 1, 2, 3, or 4,
s is 0, 1, 2, 3, 4, 5, 6, 7, 8, 9, 10, 11 or 12,
and the amino acid of formulae (32) and (33) is optionally substituted.

In some embodiments, the amino acid of formulae (32) and (33) is substituted with R^{X11}—CO—NH— at an α-carbon atom which is covalently bound to the COOH-group in formulae (32) and (33), wherein R^{X11} is selected from the group consisting of (C₁-C₁₀)alkyl, (C₅-C₁₀)aryl, and (C₁-C₅)alkyl-(C₅-C₁₀)aryl. Preferably, R^{X11} is methyl.

In preferred embodiments, X31 and X32 are each and individually a residue of an amino acid selected from the group consisting of 1,13-diamino-4,7,10-trioxatridecan-succinamic acid [Ttds], 8-amino-3,6-dioxaoctanoic acid [O2Oc], 2-(4-(amino)piperidin-1-yl)acetic acid [APac], 4-Carboxymethyl piperazine [PPac], 4-trans-aminomethylcyclohexane carboxylic acid [4Amc]β-Alanine [Bal], 7-Aminobutyric acid [Gab], 5-amino pentanoic acid [Ava], 6-aminohexanoic acid [Ahx], 3-aminomethyl-benzoic acid [Mamb], 4-aminomethyl-benzoic acid [Pamb] and an α-amino acid of formula (35):

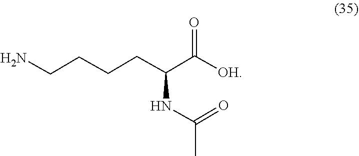

Xaa6 may be a residue of an amino acid which is selected from the group consisting of a polar L-α-amino acid, an aromatic L-α-amino acid, an aliphatic L-α-amino acid, an S-alkylated cysteine, an oxidized form of an S-alkylated cysteine, and a residue of an amino acid according to formula (3),

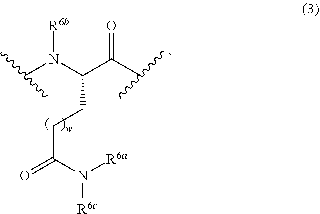

wherein
   R^{6a} is selected from the group consisting of H a moiety comprising a —(C₅-C₁₀)aryl, (C₁-C₈)alkyl, and (C₁-C₈)alkyl-(C₅-C₁₀)aryl,
   R^{6b} is selected from the group consisting of H or methyl,
   R^{6c} is H or (C₁-C₆)alkyl, and
   w is 0 or 1.

In some embodiments, Xaa6 is a residue of a polar N-methylated L-α-amino acid.

In some embodiments, Xaa6 is a residue of a aliphatic L-α-amino acid, wherein the aliphatic L-α-amino acid is preferably Ala.

In some embodiments, Xaa6 is a residue of an S-alkylated cysteine.

In some embodiments, Xaa6 is a residue of an oxidized form of an S-alkylated cysteine, preferably a sulfoxide or sulfone of an S-alkylated cysteine (meaning that the S atom present in the side chain of the S-alkylated cysteine is oxidized to form a sulfoxide or sulfone group).

In some embodiments, Xaa6 is a residue of an amino acid according to formula (3) and R^{6a} is selected from the group consisting of (C₁-C₁₀)alkyl, (C₅-C₁₀)aryl, (C₁-C₅)alkyl-(C₅-C₁₀)aryl and (C₃-C₇)cycloalkyl-(C₅-C₁₀)aryl. Preferably, R^{6c} is (C₁-C₄)alkyl.

In preferred embodiments, Xaa6 is a residue of an amino acid which is selected from the group consisting of Ala, Asp, Asn, (S)-homoserine [Hse], Gln, Glu, Lys, (S)-ornithine [Orn], (S)-2,4-diaminobutyric acid [Dab], N-Methyl-Asp, (S)-benzylcysteine [C(Bzl)], (S)-2-amino-3-(quinolin-2-ylmethylsulfanyl)-propionic acid [C(2Quyl)], (S)-benzyl-cysteine-sulfone [Eem], (S)-4-benzyloxy-L-phenylalanine [Tyr(Bzl)], and (S)-2-amino-4-[(naphthalen-1-ylmethyl)-carbamoyl]-butyric acid [E(NIMe2Nph)]. More preferably, Xaa6 is a residue of Asp.

Xaa6 may be a residue of an L-α-amino acid comprising, in addition to an amino group and a carboxy group attached to an α-C atom, a functional group FG3 forming a covalent linkage B2 with a functional group FG4 of Xaa11, wherein Xaa11 is a residue of an α-amino acid comprising, in addition to an amino group and a carboxy group attached to an α-C atom, the functional group FG4, wherein a bicyclic peptide of formula (1c) is formed:

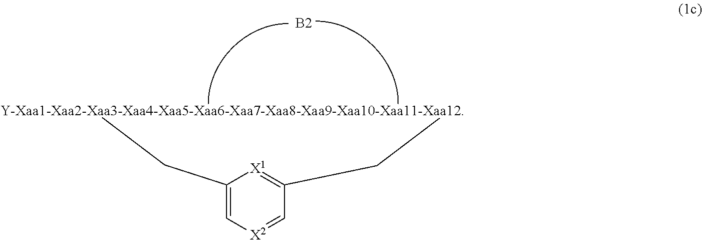

The covalent linkage B2 is preferably selected from the group consisting of an amide linkage, a disulfide linkage, a thioether linkage, a thiourea linkage, a triazole linkage, a carbamate linkage, an amine linkage, a sulfonamide linkage, an ester linkage, a thioester linkage, an ether linkage, a urea linkage and a hydrocarbon linkage, more preferably from the group consisting of an amide linkage or a disulfide linkage.

In some embodiments, the functional group FG3 of Xaa6 forming the covalent linkage B2 with the functional group FG4 of Xaa11 may be selected from the group consisting of NH₂, NH—, COOH, activated carboxylic acid, chloro, bromo, iodo, SH, OH, SOOH, activated sulfonic acid, sulfonic acid ester, Michael acceptors, isocyanate, isothiocyanate, azide, alkene, and alkyne. Xaa6 is preferably a residue of an α-amino acid selected from the group consisting of (S)-2,3-diaminopropionic acid [Dap], (S)-2,4-diaminobutyric acid [Dab], (S)-ornithine [Orn], Lys, Cys, (S)-homocysteine [Hcy], (R)-penicillamine [Pen], Asp and Glu.

In some embodiments, the functional group FG4 of Xaa11 forming the covalent linkage B2 with a functional group FG3 of Xaa6 is selected from the group consisting of NH₂, NH—, COOH, activated carboxylic acid, chloro, bromo, iodo, SH, OH, SOOH, activated sulfonic acid, sulfonic acid ester, Michael acceptors, isocyanate, isothiocyanate, azide, alkene and alkyne. Xaa11 is preferably a residue of an L-α-amino acid selected from the group consisting of (S)-2,3-diaminopropionic acid [Dap], (S)-2,4-diaminobutyric acid [Dab], (S)-ornithine [Orn], Lys, Cys, (S)-homocysteine [Hcy], (R)-penicillamine [Pen] Asp, D-asp, D-glu and Glu.

Xaa7 is a residue of an amino acid which is selected from the group consisting of an aromatic amino acid, such as a heteroaromatic L-α-amino acid, and a substituted aromatic amino acid, such as a substituted heteroaromatic L-α-amino acid. Preferably, Xaa7 is a residue of an aromatic amino acid which may be substituted at the aromatic ring system with at least one substituent. In some embodiments, the aromatic amino acid is selected from the group consisting of (S)-3-benzothienyl alanine [Bta], Trp and Phe.

In some embodiments, Xaa7 is a residue of an amino acid selected from the group consisting of substituted (S)-3-benzothienyl alanine [Bta], substituted Trp, substituted Phe, a modified 3-aminophenyl alanine [Af3(R^{7c})] of formula (4a):

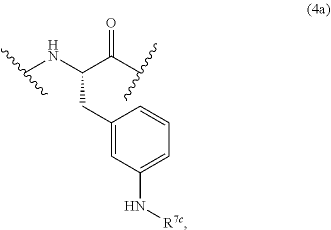

and
a modified 4-aminophenyl alanine [Aph(R^{7d})] of formula (4b):

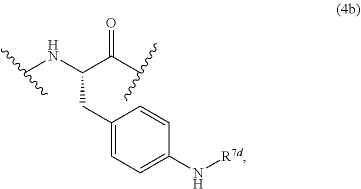

wherein
   the substituted Bta and the substituted Trp are each and individually substituted at the aromatic ring with a substituent selected from the group consisting of a halogen, methyl, and OH,
   under the proviso that, in the substituted Bta and the substituted Trp, one or two of the aromatic carbon atoms may be replaced by an N-atom,
   the substituted Phe is substituted at the aromatic ring with one, two or three substituents, wherein each and any of the substituents is individually and independently selected from the group consisting of a halogen, methyl, OH, NH₂, O—R^{7a}, wherein
      R^{7a} is (C₁-C₆)alkyl, and
wherein, in formula (4a),
   R^{7c} is —CO—R^{7e},
   wherein R^{7e} is selected from the group consisting of (C₁-C₅)alkyl, (C₅-C₁₀)aryl and (C₅-C₁₀)heterocyclyl, wherein
      (C₁-C₅)alkyl is optionally substituted with a substituent selected from the group consisting of OH, SO₂NH₂, SO₂NH—R^{7f}, CO(NHOH), COOH, CONH₂ and NH₂,
      one alkyl carbon atom of (C₁-C₅)alkyl is optionally replaced by an atom or moiety each selected from the group consisting of an ether oxygen and a sulfone (SO₂) moiety,
      (C₅-C₁₀)aryl is optionally substituted with a substituent selected from the group consisting of a halogen, OH, SO₂NH₂, SO₂NH—R^{7f}, CO(NHOH), COOH, CONH₂ and NH₂, and
      (C₅-C₁₀)heterocyclyl is optionally substituted with a substituent selected from the group consisting of a halogen, OH, SO₂NH₂, SO₂NH—R^{7f}, NH—SO—NH₂, CO(NHOH), COOH, CONH₂ and NH₂,
   wherein
   R^{7f} is (C₁-C₄)alkyl,
wherein, in formula (4b),
   R^{7d} is —CO—R^{7g},
   wherein
   R^{7g} is (C₂-C₅)alkyl, (C₅-C₁₀)aryl and (C₅-C₁₀)heterocyclyl,
   wherein
      (C₁-C₈)alkyl is optionally substituted with a substituent selected from the group consisting of OH, SO₂NH₂, SO₂NH—R^{7h}, CO(NHOH), COOH, CONH₂ and NH₂,
      one alkyl carbon atom of (C₂-C₅)alkyl is optionally replaced by an atom or moiety each selected from the group consisting of an ether oxygen and a sulfone (SO₂) moiety,
      (C₅-C₁₀)aryl is optionally substituted with a substituent selected form the group consisting of a halogen, OH, SO₂NH₂ SO₂NH—R^{7h}, CO(NHOH), COOH, CONH₂ and NH₂, and
      (C₅-C₁₀)heterocyclyl is optionally substituted with a substituent selected from the group consisting of a halogen, OH, SO₂NH₂, SO₂NH—R^{7h}, NH—SO—NH₂, CO(NHOH), COOH, CONH₂ and NH₂, and
   wherein
   R^{7h} is (C₁-C₄)alkyl.

In preferred embodiments, Xaa7 is a residue of an amino acid, wherein the amino acid is selected from the group consisting of:
modified 3-aminophenyl alanine [Af3(R^{7e})] of formula (4a):

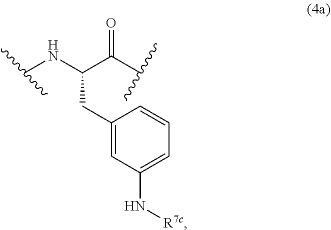

modified 4-aminophenyl alanine [Aph(R^{7d})] of formula (4b):

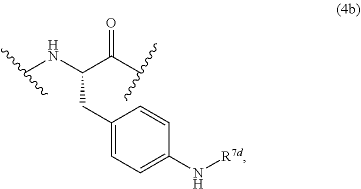

substituted Trp, substituted (S)-3-benzothienyl alanine [Bta], (S)-3-(1-naphthyl)alanine [1Ni], (S)-4-benzyloxy-L-phenylalanine [Tyr(Bzl)], Tyr, substituted Phe and (S)-benzylcysteine [Cys(Bzl)], preferably Xaa7 is a residue of modified 3-aminophenyl alanine [Af3(R^{7c})] of formula (4a) or of modified 4-aminophenyl alanine [Aph(R^{7d})] of formula (4b).

In preferred embodiments, Xaa7 is a residue of an amino acid selected from the group consisting of: D/L-1-methyltryptophane [1MW], D/L-7-methyltryptophane [7MW], 5-chloro-tryptophane [5Clw], DL-5-methyl-tryptophane [Egc], substituted [Bta], (S)-4-benzyloxy-L-phenylalanine [Tyr(Bzl)], (S)-3-(1-naphthyl)alanine [1Ni], (2S)-2-amino-3-[3-(trifluoromethyl)phenyl]propanoic acid [Mtf], (2S)-2-amino-3-[4-(trifluoromethyl)phenyl]propanoic acid [Ptf], (S)-3,4-dichlorophenylalanine [Eaa], 4-(tert-butyl)-phenylalanine [Eap], (2S)-2-amino-3-(4-iodophenyl)propanoic acid [Pif], (S)-biphenylalanine [Bip], (S)-3,3-diphenylalanine [Dip], (S)-benzylcysteine [Cys(Bzl)], the modified 3-aminophenyl alanine [Af3(R^{7c})] of formula (4a) and modified 4-aminophenyl alanine [Aph(R^{7d})] of formula (4b), wherein R^{7c} is selected from the group consisting of:

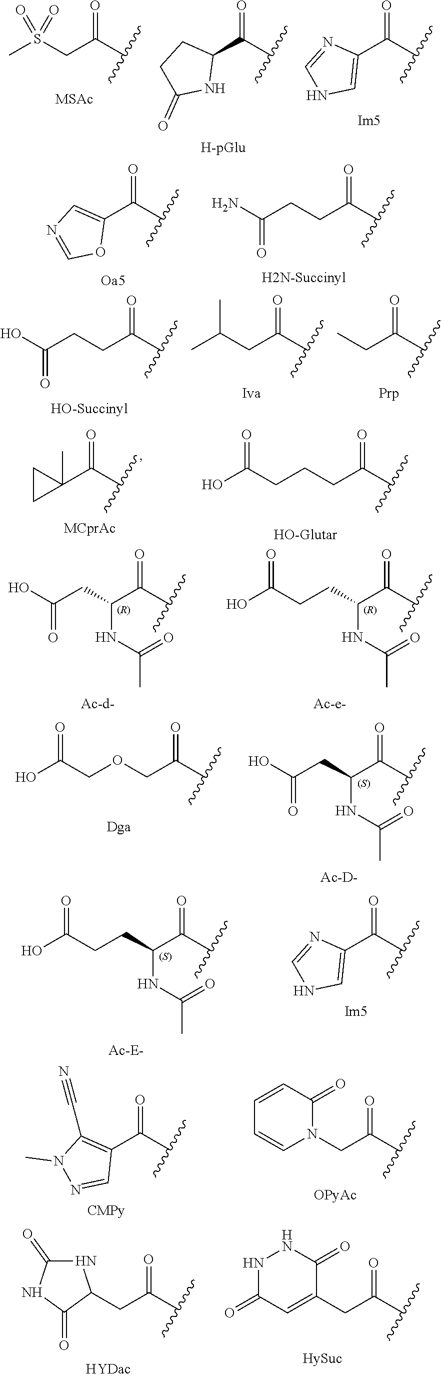

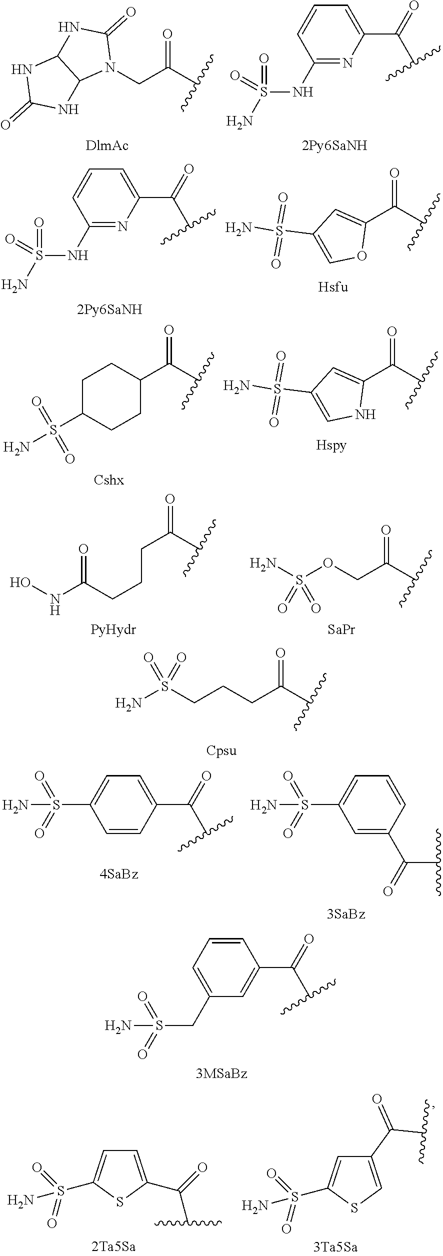

preferably R^{7c} is selected from the group consisting of:

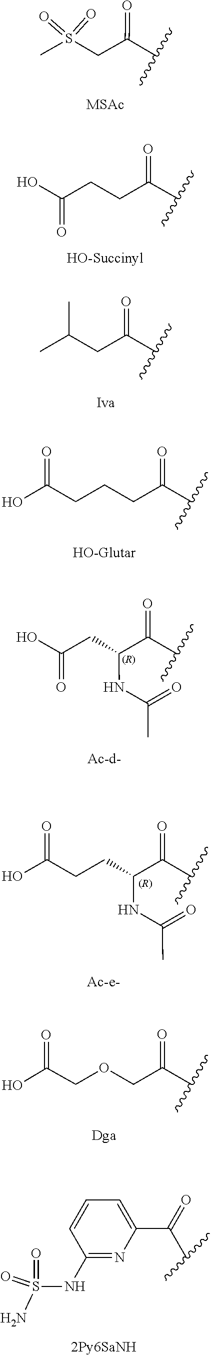

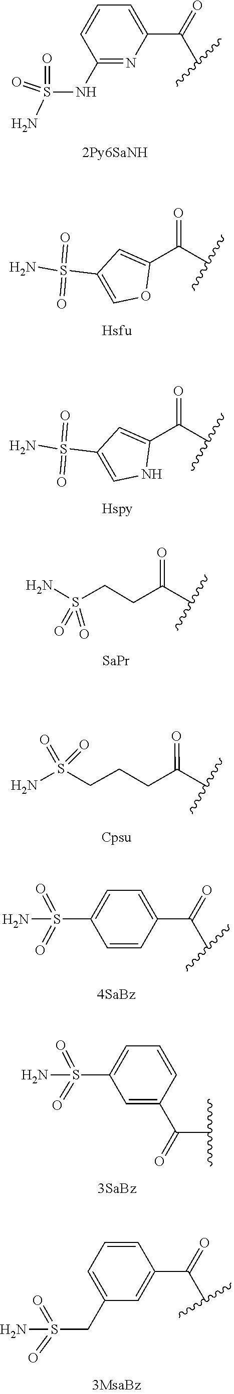

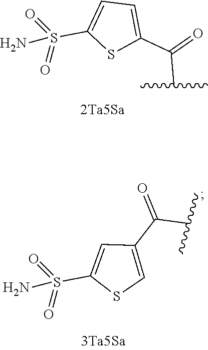

wherein R^{7d} is selected from the group consisting of:

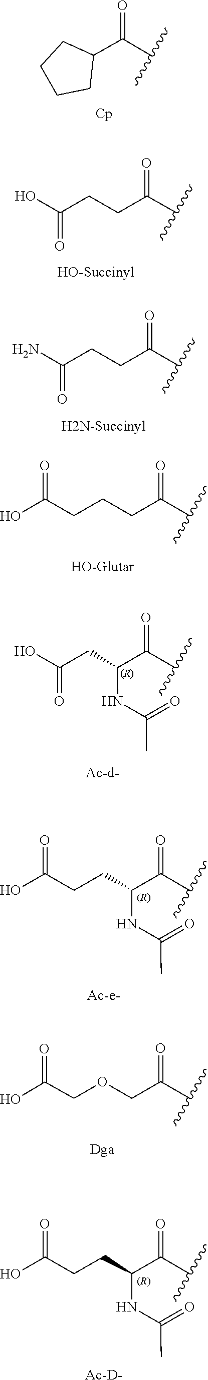

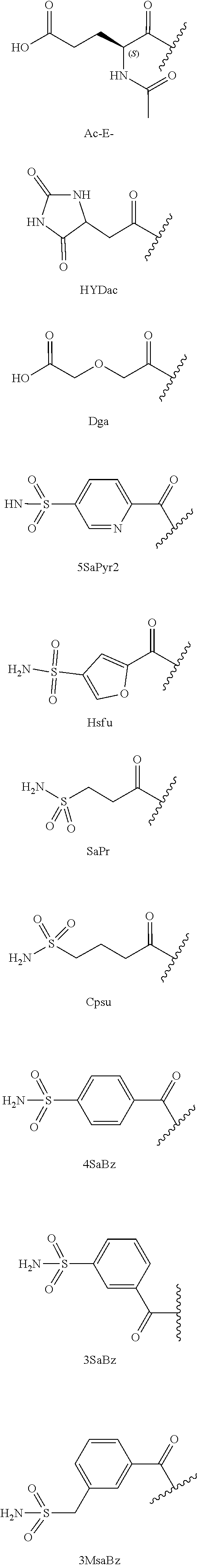

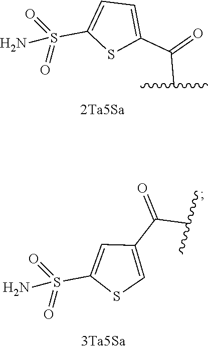 [SMILES: *C(=O)c1ccc(S(N)(=O)=O)s1 ; *C(=O)c1csc(S(N)(=O)=O)c1]

preferably R^{7d} is selected from the group consisting of:

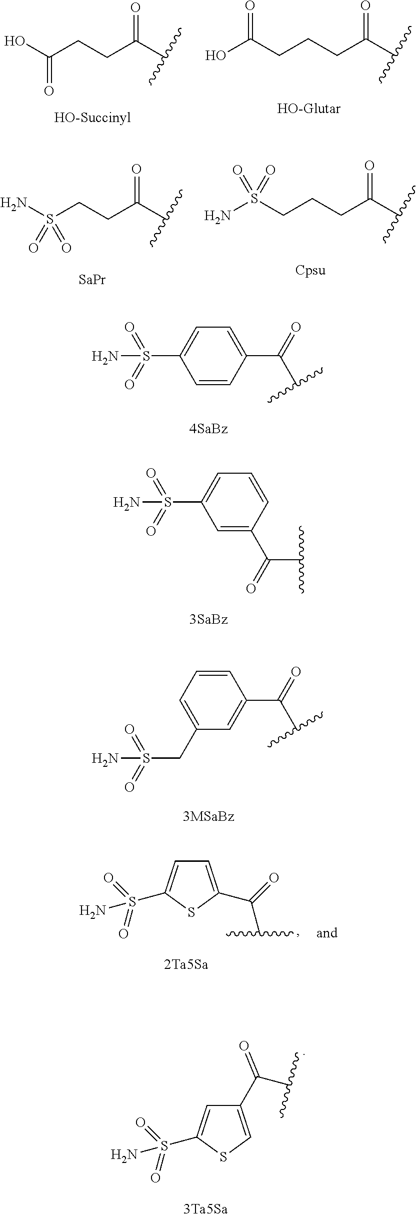

In more preferred embodiments, Xaa7 is a residue of an amino acid selected from the group consisting of the modified 3-aminophenyl alanine [Af3(R⁷)] of formula (4a) and the modified 4-aminophenyl alanine [Aph(R^{7d})] of formula (4b), wherein
R^{7c} is selected from the group consisting of:

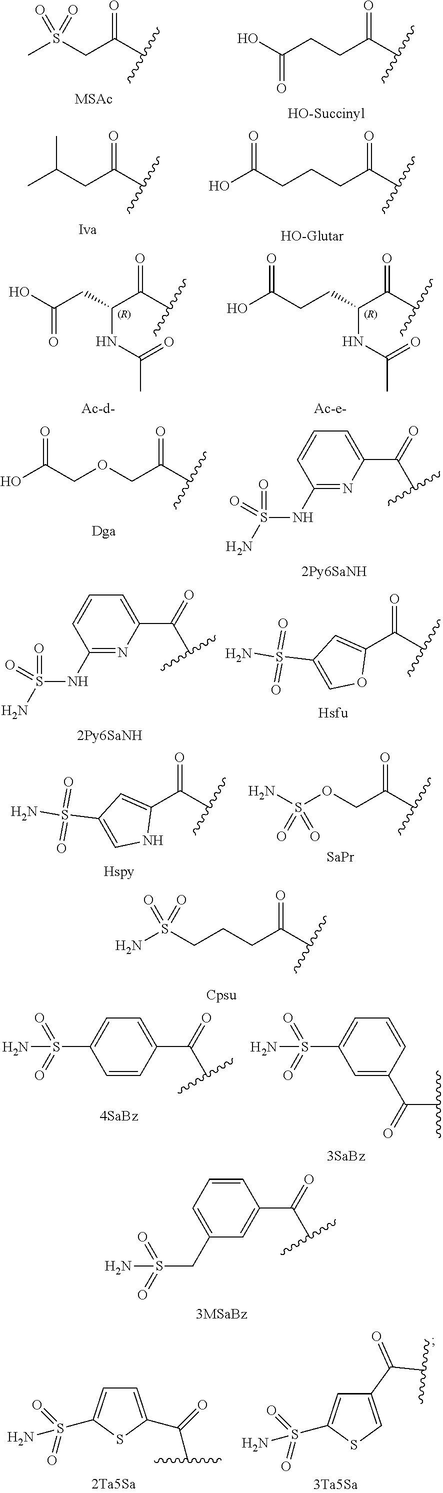

and wherein R^{7d} is selected from the group consisting of:

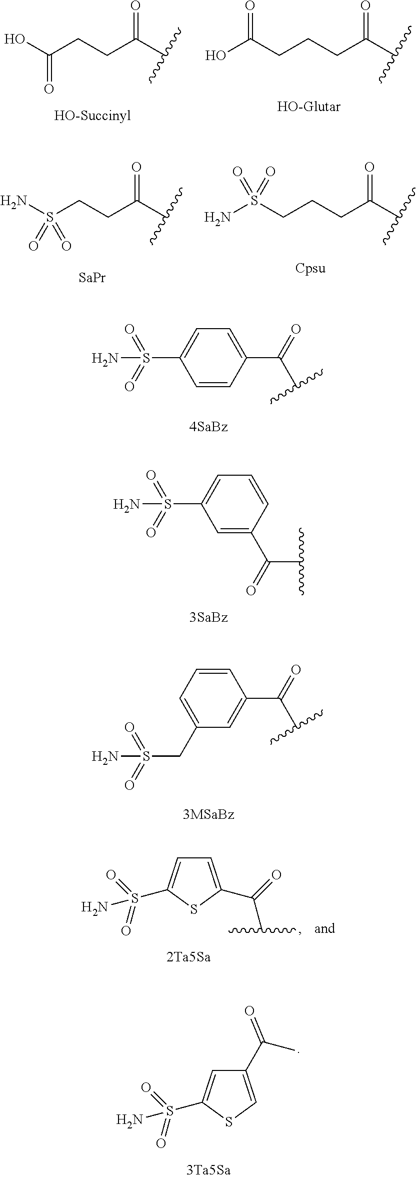

Most preferably, Xaa7 is a residue of the modified 3-aminophenyl alanine [Af3(R^{7c})] of formula (4a), wherein R^{7c} is

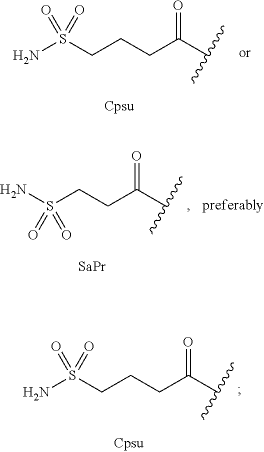 [SMILES: *C(=O)CCCS(N)(=O)=O ; *C(=O)CCS(N)(=O)=O ; *C(=O)CCCS(N)(=O)=O]

Xaa7 is a residue of the modified 4-aminophenyl alanine [Aph(R^{7d})] of formula (4b), wherein R^{7d} is

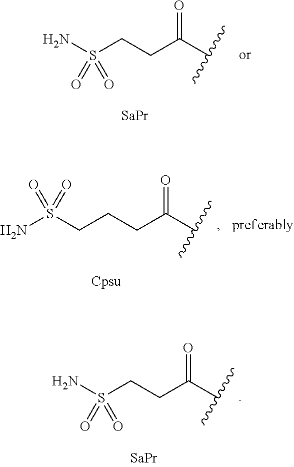 [SMILES: *C(=O)CCS(N)(=O)=O ; *C(=O)CCCS(N)(=O)=O ; *C(=O)CCS(N)(=O)=O]

According to the most preferred embodiments of Xaa7, it is further preferred that Xaa1 is absent.

Xaa8 is a residue of an amino acid which is selected from the group consisting of an L-α-amino acid and a cyclic α,α-dialkyl amino acid. In some embodiments, Xaa8 is a residue of an aliphatic L-α-amino acid of formula (1X) or an amino acid of formula (XI):

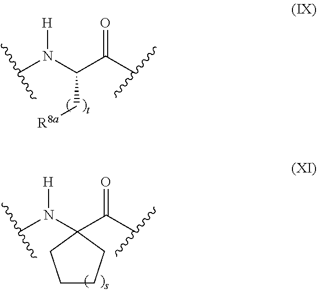

wherein
R^{8a} is selected from the group consisting of (C₁-C₄)alkyl, (C₃-C₇)cycloalkyl and H,
   t=0, 1, 2, 3, or 4
   s=0, 1, 2 or 3
wherein
in the amino acid of formula (XI) one aryl-ring is optionally annulated to a ring bond which does not include the α-C-atom, and
in the carbocyclic part of the amino acid of formula (XI) a CH₂ group which is spaced at least one carbon atom apart from the α-carbon atom is optionally replaced by an O atom or a NH group.

In preferred embodiments, Xaa8 is a residue of an amino acid selected from the group consisting of Leu, Nle, Npg, Cha, Aic, Thp, Eca, and Egz, more preferably Leu.

Xaa9 is a residue of an amino acid which is selected from the group consisting of Gly and an L-α-amino acid. In some embodiments, Xaa9 is a residue of an amino acid selected from the group consisting of Gly and an L-α-amino acid of formula (XIII):

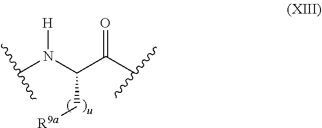

wherein
R^{9a} is selected from the group consisting of H, OH, COOH, CONH₂, N(R^{9b})₂, CONH—R^{9c}, X⁹ and —NH—CO—X⁹,
wherein
X⁹ is selected from the group consisting of (C₁-C₆)alkyl, (C₅-C₁₀)aryl and (C₃-C₁₀)heteroaryl, and X⁹ is substituted with one or two substituents each and individually selected from the group consisting of methyl, CONH₂, a halogen, NH₂ and OH;
u=1, 2, 3 or 4, wherein optionally one or two hydrogens of the 3-CH₂ group and/or of the γ-CH₂-group are each and individually substituted by methyl and/or one of the hydrogens of the β-CH₂-group is optionally substituted by OH,
R^{9b} is each and independently selected from the group consisting of (C₁-C₄)alkyl and H,
R^{9c} is selected from the group consisting of (C₁-C₈)alkyl, and (C₁-C₅)cycloalkyl optionally substituted with 1, 2, 3, 4, 5, or 6 OH-groups under the proviso and that each carbon atom is bound to no or one O or N-atom.

In preferred embodiments, Xaa9 is a residue of an amino acid selected from the group consisting of Gly, Ala, His, Thr, (S)-dimethylornithine [Dmo], and Glu(AGLU), more preferably Thr.

Xaa10 is a residue of a heteroaromatic L-α-amino acid. In some embodiments, Xaa10 is selected from the group consisting of Trp optionally substituted with a substituent selected from the group consisting of methyl, a halogen or OH, and an aza-analogue of Trp optionally substituted with methyl, a halogen or OH. Preferably, Xaa10 is a residue of an amino acid selected from the group consisting of Trp and (S)-7-aza-tryptophane [7Nw].

In some embodiments, Xaa11 may be a residue of an amino acid which is selected from the group consisting of Gly and an L-α-amino acid, wherein the L-α-amino acid is optionally bound to a moiety Z4, wherein Z4 is a moiety comprising an effector E4 and a linker moiety L4, wherein the effector E4 is preferably selected from the group consisting of:
(α) a moiety derived from a chromophore, which is preferably selected from (α1) a phosphorophore and (α2) a fluorophore, such as fluorescein or rhodamine; and
(β) a chelator optionally comprising a chelated nuclide; and
(γ) a moiety derived from a drug, preferably from a cytotoxic drug.

Preferably, Xaa11 is a residue of an amino acid which is selected from the group consisting of Gly and an L-α-amino acid, and Z4 is absent. More preferably, Xaa11 is a residue of Ser (Z4 being absent).

In some embodiments, Xaa11 may be a residue of an L-α-amino acid comprising, in addition to an amino group and a carboxy group attached to an α-C atom, the functional group FG2 forming the covalent linkage B1 with the functional group FG1 of Xaa2 such that the bicyclic peptide of formula (1b) is formed. In other embodiments, Xaa11 may be a residue of an L-α-amino acid comprising, in addition to an amino group and a carboxy group attached to an α-C atom, the functional group FG4 forming the covalent linkage B2 with the functional group FG3 of Xaa6 such that the bicyclic peptide of formula (1c) is formed.

In some embodiments, Xaa11 is a residue of an amino acid which is selected from the group consisting of Gly and an L-α-amino acid, wherein the L-α-amino acid is bound to a moiety Z4, wherein Z4 is a moiety comprising an effector E4 and a linker moiety L4, which covalently links the effector E4 to the L-α-amino acid of Xaa11. Preferably, Xaa11 is a residue of an L-α-amino acid selected from the group consisting of Glu, Gln, and an L-α-amino acid of formula (XI):

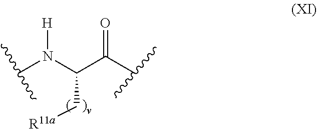

wherein
v=1, 2, 3 or 4,
R^{11a} is selected from the group consisting of H, OH, COOH, CONH₂, NH— (C═NH)—NH₂, N(R^{11b})₂, CONH—R^{11c}, —CO(Z4), X¹³ and —NH—CO—X¹³, NH—CO(Z4), O—CO(Z4), Z4 and NH—CS—Z4, wherein
X¹³ is selected from the group consisting of (C₁-C₆)alkyl, (C₅-C₆)aryl and (C₃-C₅)heteroaryl and X¹³ is optionally substituted with one or two substituents each and individually selected from the group consisting of methyl, CONH₂, a halogen, NH₂ and OH,
R^{11b} is each and independently selected from the group consisting of (C₁-C₄)alkyl and H, and
   optionally one or two hydrogens of the β-CH₂ group and/or of the γ-CH₂-group in formula (XI) are each and individually substituted by methyl, and
   one of the hydrogens of the β-CH₂-group in formula (XI) is optionally substituted by OH.

In preferred embodiments, Xaa11 is bound to Z4 and is a residue of an amino acid selected from the group consisting of Ala, Ser, Gly, Arg, Lys, (S)-dimethylornithine [Dmo], and Glu(AGLU). In this connection, it is understood that the amino acid from which Xaa11 is derived from contains a functional group which enables covalent attachment of Z4 thereto. More preferably, Xaa11 is a residue of Ser (Z4 being bound to Xaa11).

In some embodiments, Xaa11 includes a functional group FG5 different from the carboxyl group and the amino group attached to the α-C atom of Xaa11, and the linker moiety L4 covalently links the effector E4 to the functional group FG5 of the L-α-amino acid of Xaa11. Preferably, Xaa11 is a residue of an L-α-amino acid of formula (XI) and the functional group FG5 is provided by R^{11a}. In particular, the linker moiety L4 may provide (a) a first amino group forming a covalent bond with the functional group FG5 of the L-α-amino acid of Xaa11 and (b) a second amino group forming a covalent bond to the effector E4.

In some embodiments, the linker moiety L4 is either X41 or a residue selected from the group consisting of X41-X42 and X42-X41, wherein
X41 is a residue of a diamine providing a first amino group and a second amino group,
X42 is a residue of an amino acid providing an amino group and a carboxy group,
X41-X42 is a residue of a diamine, wherein the diamine provides a first amino group and a second amino group,
wherein the first amino group is the first amino group of X41,
   the second amino group is the amino group of X42, and
   the second amino group of X41 forms an amide bond with the carboxy group of X42, and
X42-X41 is a residue of a diamine, wherein the diamine provides a first amino group and a second amino group,
wherein the first amino group is the amino group of X42,
   the second amino group is the second amino group of X41, and
   the carboxy group of X42 forms an amide bond with the first amino group of X41.

In some embodiments, X41 is a residue of a linear or a cyclic diamine. In particular, Xaa11 may be a residue of an L-α-amino acid of formula (XI) and R^{11a} is selected from the group consisting of —CO(Z4), —NH—CO(Z4), —O—CO(Z4), —Z4 and —NH—CS—Z4. Preferably, L4 is covalently attached to the carbonyl or thiocarbonyl carbon atom comprised in R^{11a} by means of an amide bond.

In preferred embodiments, X41 is a residue of a diamine which is selected from the group consisting of a diamine of any one of formulae (35) to (37):

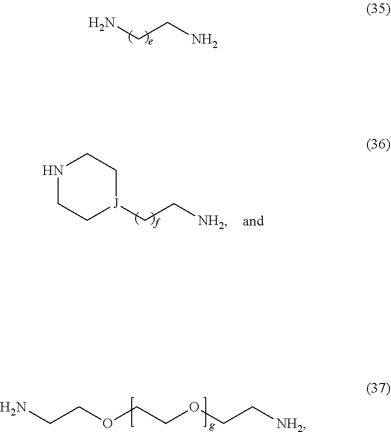

wherein
   e is 1, 2, 3, 4, 5, 6, 7, 8, 9, or 10,
   f is 0, 1, 2, 3, 4, 5 or 6,
   g is 0, 1, 2, 3, 4, 5, 6, 7, 8, 9, 10, 11, or 12,
   the diamine of any one of formulae (35) and (36) is optionally substituted with —CONH₂, and
   J is selected from the group consisting of CH and N.

In the diamine of any one of formulae (35) and (36), the carbon atom which is substituted with a nitrogen atom may be further substituted with —CONH₂.

In more preferred embodiments, X41 is a residue of a diamine selected from the group consisting of 1,3-diaminopropane [Apr], 1,5-diaminopentane [Ape], diaminobutane and ethylendiamine.

In more preferred embodiments, X42 is a residue of an amino acid selected from the group consisting of 1,13-diamino-4,7,10-trioxatridecan-succinamic acid [Ttds], 8-amino-3,6-dioxaoctanoic acid [O2Oc], 2-(4-(amino)piperidin-1-yl)acetic acid [APac], 4-carboxymethyl piperazine [PPac], 4-trans-aminomethylcyclohexane carboxylic acid [4Amc] and an amino acid of any one of formulae (32), (33) and (34):

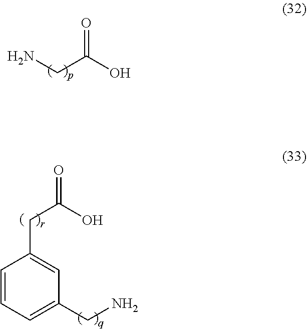

and the ortho- and para-substituted isomers thereof, and

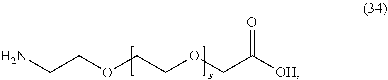

wherein
p is 2, 3, 4, 5, 6, 7, 8, 9, or 10,
q is 0, 1, 2, 3, or 4,
r is 0, 1, 2, 3, or 4,
s is 0, 1, 2, 3, 4, 5, 6, 7, 8, 9, 10, 11 or 12, and the amino acid of formulae (32) and (33) is optionally substituted.

The amino acid of formulae (32) and (33) may be substituted with R^{X11}—CO—NH— at the α-carbon atom which is covalently bound to the COOH-group in each one of formulae (32) and (33), wherein R^{X11} is selected from the group consisting of (C₁-C₁₀)alkyl, (C₅-C₁₀)aryl, and (C₁-C₈)alkyl-(C₅-C₁₀)aryl. Preferably, R^{X11} is methyl.

Most preferably, X42 is a residue of an amino acid selected from the group consisting of 1,13-diamino-4,7,10-trioxatridecan-succinamic acid [Ttds], 8-amino-3,6-dioxaoctanoic acid [O2Oc], 2-(4-(amino)piperidin-1-yl)acetic acid [APac], 4-carboxymethyl piperazine [PPac], 4-trans-aminomethylcyclohexane carboxylic acid [4Amc], β-alanine [Bal], γ-aminobutyric acid [Gab], 5-amino pentanoic acid [Ava], 6-aminohexanoic acid [Ahx], 3-aminomethyl-benzoic acid [Mamb], 4-aminomethyl-benzoic acid [Pamb] and an amino acid of formula (35):

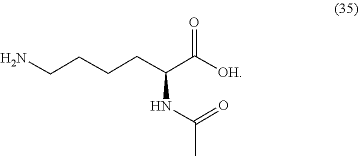

Xaa12 is a residue of an amino thiol of formula (XII):

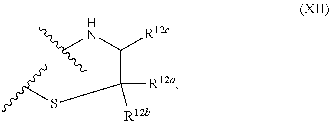

preferably of formula (XIIa):

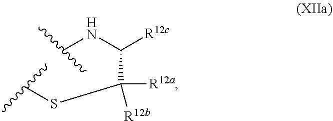

wherein
the NH of formula (XII) is bound to Xaa11;
R^{12a} and R^{12b} are each and independently selected from the group consisting of H and CH₃, preferably H;
R^{12c} is selected from the group consisting of —COOH, CONH₂, —CO—Z6 and —CH₂—Z6, wherein Z6 comprises a linker moiety L6 and an effector E6.

The effector E6 is preferably selected from the group consisting of:
(α) a moiety derived from a chromophore, which is preferably selected from (α1) a phosphorophore and (α2) a fluorophore, such as fluorescein or rhodamine; and
(β) a chelator optionally comprising a chelated nuclide; and
(γ) a moiety derived from a drug, preferably from a cytotoxic drug.

Most preferably, both R^{12a} and R^{12b} are H and Xaa12 is in the (R)-configuration.

In some embodiments, R^{12c} is selected from the group consisting of —COOH and —CONH2.

In some embodiments, R^{12c} is selected from the group consisting of —CO—Z6 and —CH₂—Z6, wherein Z6 is a moiety comprising an effector E6 and a linker moiety L6, which covalently links the effector E6 to a carbon atom of R^{12c}. Preferably, R^{12c} is —CO—Z6 and the linker moiety L6 provides (a) a first amino group forming a covalent bond to carbonyl carbon atom of R^{12c}, and (b) a second amino group forming a covalent bond to the effector.

In some embodiments, the linker moiety L6 is either X61 or a residue selected from the group consisting of X61-X62 and X62-X61, wherein
X61 is a residue of a diamine providing a first amino group and a second amino group,
X62 is a residue of an amino acid providing an amino group and a carboxy group,
X61-X62 is a residue of a diamine, wherein the diamine provides a first amino group and a second amino group,
wherein the first amino group is the first amino group of X61,
   the second amino group is the amino group of X62, and
   the second amino group of X61 forms an amide bond with the carboxy group of X62, and
X62-X61 is a residue of a diamine, wherein the diamine provides a first amino group and a second amino group,
wherein the first amino group is the amino group of X62,
   the second amino group is the second amino group of X61, and
   the carboxy group of X62 forms an amide bond with the first amino group of X61.

Preferably, X61 is a residue of a diamine which is selected from the group consisting of a diamine of any one of formulae (35-37):

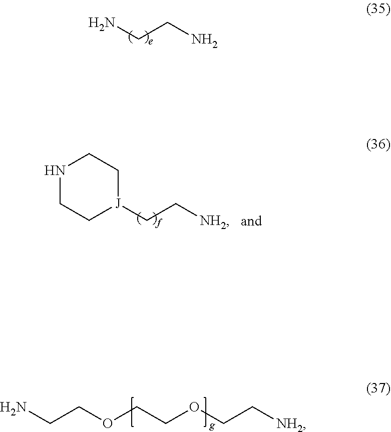

wherein
   e is 1, 2, 3, 4, 5, 6, 7, 8, 9, or 10,
   f is 0, 1, 2, 3, 4, 5 or 6,
   g is 0, 1, 2, 3, 4, 5, 6, 7, 8, 9, 10, 11, or 12, and
   the diamine of any one of formulae (35) and (36) is optionally substituted with —CONH₂, and wherein J is selected from the group consisting of CH and N.

In the diamine of any one of formulae (35) and (36), the carbon atom which is substituted with a nitrogen atom may be further substituted with —CONH₂.

More preferably, X61 is a residue of a diamine selected from the group consisting of 1,3-diaminopropane [Apr], 1,5-diaminopentane [Ape], diaminobutane, ethylenediamine, a diamine of formula (39), and a diamine of formula (40)

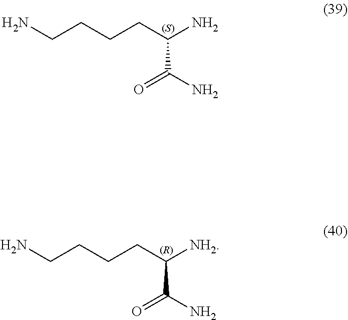

In some embodiments, X62 is a residue of an amino acid selected from the group consisting of 1,13-diamino-4,7,10-trioxatridecan-succinamic acid [Ttds], 8-amino-3,6-dioxaoctanoic acid [O2Oc], 2-(4-(amino)piperidin-1-yl)acetic acid [APac], 4-carboxymethyl piperazine [PPac], 4-trans-aminomethylcyclohexane carboxylic acid [4Amc] and an amino acid according to any one of formulae (32)-(33):

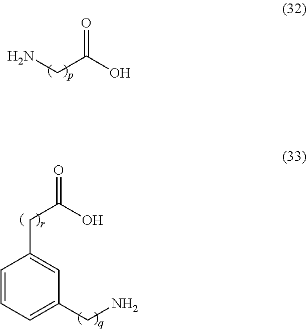

and the ortho- and para-substituted isomers thereof, and

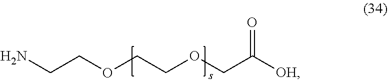

wherein
p is 2, 3, 4, 5, 6, 7, 8, 9, or 10,
q is 0, 1, 2, 3, or 4,
r is 0, 1, 2, 3, or 4,
s is 0, 1, 2, 3, 4, 5, 6, 7, 8, 9, 10, 11 or 12,
the amino acid of formula (32) and of formula (33) is each optionally substituted.

The amino acid of formula (32) and of formula (33) may each be substituted with R^{X11}—CO—NH— at the α-carbon atom which is covalently bound to the COOH-group in formulae (32) and (33), wherein R^{X11} is (C₁-C₁₀)alkyl, (C₅-C₁₀)aryl, and (C₁-C₈)alkyl-(C₅-C₁₀)aryl. Preferably, R^{X11} is methyl.

In preferred embodiments, X62 is a residue of an amino acid selected from the group consisting of 1,13-diamino-4,7,10-trioxatridecan-succinamic acid [Ttds], 8-amino-3,6-dioxaoctanoic acid [O2Oc], 2-(4-(amino)piperidin-1-yl)acetic acid [APac], 4-Carboxymethyl piperazine [PPac], 4-trans-aminomethylcyclohexane carboxylic acid [4Amc], β-alanine [Bal], γ-aminobutyric acid [Gab], 5-amino pentanoic acid [Ava], 6-aminohexanoic acid [Ahx], 3-aminomethyl-benzoic acid [Mamb], 4-aminomethyl-benzoic acid [Pamb] and an amino acid of formula (35):

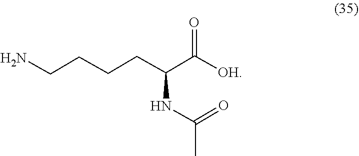

Most preferably, X62 is a residue of an amino acid selected from the group consisting of 1,13-diamino-4,7,10-trioxatridecan-succinamic acid [Ttds], 8-amino-3,6-dioxaoctanoic acid [O2Oc], 3-aminomethyl-benzoic acid [Mamb], 4-aminomethyl-benzoic acid [Pamb].

In formula (1a), X¹ and X² are each and independently selected from the group consisting of C—H and N. Preferably, at least one of X¹ and X² is C—H and N. Most preferably, both X¹ and X2 are C—H.

However, in more preferred embodiments, the compound of the invention contains only one effector selected from E1, E3, E4, and E6, which effector may be attached to the compound via a linker moiety L1, L3, L4 or L6.

According to the present invention the compound of the invention may comprise one or more effectors (i.e., E1, E3, E4, and E6) which is/are either directly or by means of a linker attached to the compound of the invention. It is, however, preferred that the compound of the invention comprises not more than two effectors, and more preferably only one effector. Most preferably, such one effector is comprised by the N-terminal group Y.

In preferred embodiments, the compound of the present invention is selected from the group consisting of:

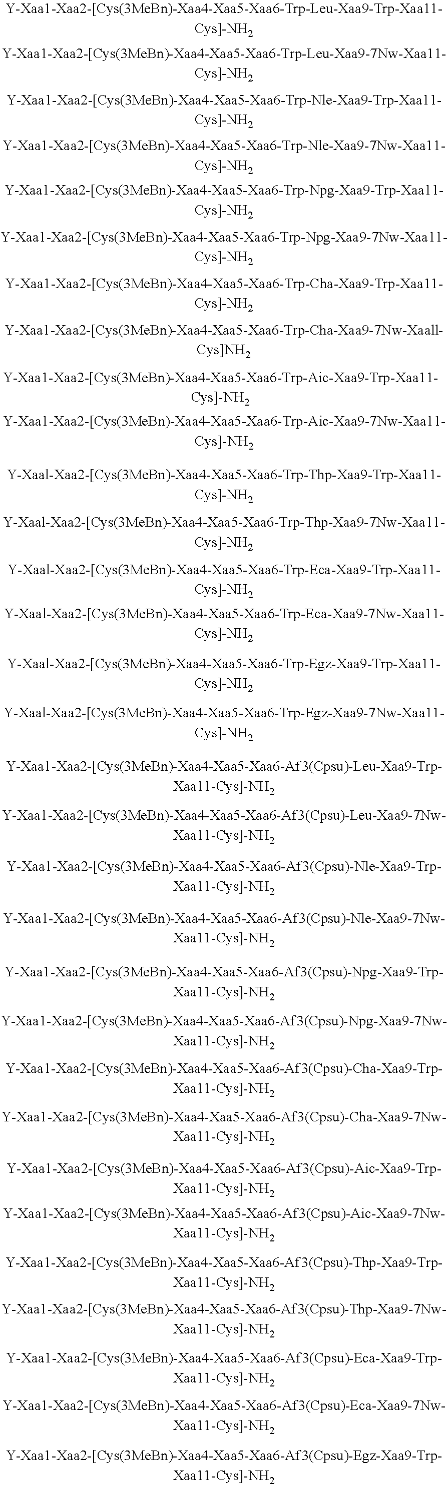

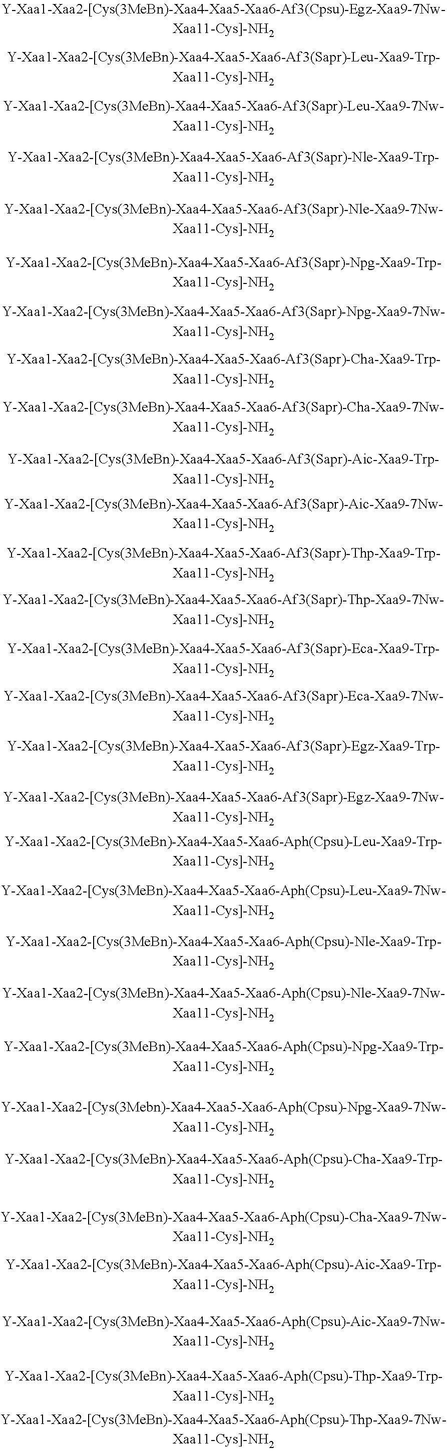

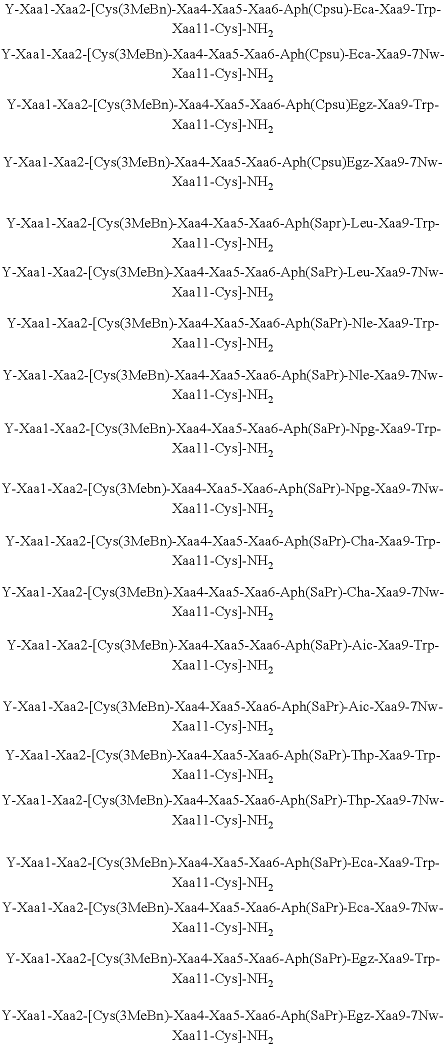

wherein Y, Xaa1, Xaa2, Xaa4, Xaa5, Xaa6, Xaa9 and Xaa11 are as defined above, and wherein preferably at least one—e.g., two, three, four, or more than four—of Y, Xaa1, Xaa2, Xaa4, Xaa5, Xaa6, Xaa9 and Xaa11 is/are defined as follows:
(a) Y is a group selected from (a1) Ac, (a2) a moiety comprising an effector E1, and (a3) Z1;
(b) Xaa1 is absent, or represents a residue of an L-α-amino acid selected from the group consisting of Val, Ile, Tle, Ser and Thr;
(c) Xaa2 is (cl) a residue of an L-α-amino acid selected from the group consisting of Tyr, Nmy, Phe, Gln, Arg, Dmo, Ser, Thr, Asp, Glu and Glu(AGLU), or (c2) is a residue of an L-α-amino acid comprising a functional group FG1 forming a covalent linkage B1 with a functional group FG2 of Xaa11, which is selected from the group consisting of Dap, Dab, Orn, Lys, Cys, Hcy, Pen, Asp and Glu;
(d) Xaa4 is a residue of an L-α-amino acid selected from the group consisting of Ala, Ser, Hse, Nms, Gln, Asn, Glu, Asp, Dmo and Glu(AGLU);
(e) Xaa5 is a residue of an amino acid wherein Z3 is absent, which is selected from the group consisting of Gly, Nmg, D-ala, D-pro, D-pip, D-aze, Nma and Aib;
(f) Xaa6 is (f1) a residue of an L-α-amino acid selected from the group consisting of Ala, Asp, Asn, Hse, Gln, Glu, Lys, Orn, Dab, N-methyl-Asp, C(Bzl), C(2Quuyl), Eem, Tyr(Bzl) and E(NHMe2Nph), or (f2) is a residue of an L-α-amino acid comprising a functional group FG3 forming a covalent linkage B2 with a functional group of Xaa11, which is selected from the group consisting of Dap, Dab, Orn, Lys, Cys, Hcy, Pen, Asp and Glu;
(g) Xaa9 is a residue of an amino acid selected from the group consisting of Gly, Ala, His, Thr, Dmo and Glu(AGLU);
(h) Xaa11 is one selected from (h1) a residue of Ser, (h2) a residue of an L-α-amino acid comprising the functional group FG2 forming the covalent linkage B1 with the functional group FG1 of Xaa2, which is selected from the group consisting of Dap, Dab, Orn, Lys, Cys, Hcy, Pen, Asp and Glu, and (h3) a residue of an L-α-amino acid comprising the functional group FG4 forming the covalent linkage B2 with the functional group FG3 of Xaa6, which is selected from the group consisting of Dap, Dab, Orn, Lys, Cys, Hcy, Pen, Asp and Glu.

In further preferred embodiments, in the above formulae, at least one—e.g., two, three, four or more than four—of Xaa1, Xaa2, Xaa4, Xaa5, Xaa6, Xaa9 and Xaa11 is/are defined as follows while Y is preferably as defined above under item (a):
(b) Xaa1 is absent, or represents a residue of Val;
(c) Xaa2 is (cl) a residue of an L-α-amino acid selected from the group consisting of Tyr, Nmy, Gln, Arg, Dmo and Ser, or (c2) is a residue of an L-α-amino acid comprising a functional group FG1 forming a covalent linkage B1 with a functional group FG2 of Xaa11, which is selected from the group consisting of Dap, Dab, Orn, Lys, Cys, Hcy, Pen, Asp and Glu;
(d) Xaa4 is a residue of an L-α-amino acid selected from the group consisting of Ala, Ser, Glu, Gln and Hse;
(e) Xaa5 is a residue of an amino acid wherein Z3 is absent, which is selected from the group consisting of Gly, Nmg, D-ala, D-pro, D-pip, D-aze, Nma and Aib;
(f) Xaa6 is (f1) a residue of an L-α-amino acid selected from the group consisting of Ala, Asp, Asn, Hse, Gln, Glu, Lys, Orn, Dab, N-methyl-Asp, C(Bzl), C(2Quuyl), Eem, Tyr(Bzl) and E(NHMe2Nph);
(g) Xaa9 is a residue of an amino acid selected from the group consisting of Gly, Ala, His, Thr, Dmo and Glu(AGLU);
(h) Xaa11 is (h1) a residue of Ser, or (h2) a residue of an L-α-amino acid comprising the functional group FG2 forming the covalent linkage B1 with the functional group FG1 of Xaa2, which is selected from the group consisting of Dap, Dab, Orn, Lys, Cys, Hcy, Pen, Asp and Glu.

In further preferred embodiments, in the above formulae, at least one—e.g., two, three, four or more than four—of Xaa1, Xaa2, Xaa4, Xaa5, Xaa6, Xaa9 and Xaa 11 is/are defined as follows while Y is preferably as defined above under item (a):
(b) Xaa1 is absent;
(c) Xaa2 is (cl) a residue of Gln, or (c2) is a residue of an L-α-amino acid comprising a functional group FG1 forming a covalent linkage B1 with a functional group FG2 of Xaa11, which Xaa2 is Glu;
(d) Xaa4 is a residue of Glu;
(e) Xaa5 is a residue of D-pro;
(f) Xaa6 is a residue of Asp;
(g) Xaa9 is a residue of Thr;
(h) Xaa11 is (h1) a residue of Ser, or (h2) a residue of an L-α-amino acid comprising the functional group FG2 forming the covalent linkage B1 with the functional group FG1 of Xaa2, which Xaa11 is Dap.

In further preferred embodiments, in the above formulae, all of Xaa1, Xaa2, Xaa4, Xaa5, Xaa6, Xaa9 and Xaa11 are defined as follows while Y is preferably as defined above under item (a):
(b) Xaa1 is absent, or represents a residue of an L-α-amino acid selected from the group consisting of Val, Ile, Tle, Ser and Thr;
(c) Xaa2 is (cl) a residue of an L-α-amino acid selected from the group consisting of Tyr, Nmy, Phe, Gln, Arg, Dmo, Ser, Thr, Asp, Glu and Glu(AGLU), or (c2) is a residue of an L-α-amino acid comprising a functional group FG1 forming a covalent linkage B1 with a functional group FG2 of Xaa11, which is selected from the group consisting of Dap, Dab, Orn, Lys, Cys, Hcy, Pen, Asp and Glu;
(d) Xaa4 is a residue of an L-α-amino acid selected from the group consisting of Ala, Ser, Hse, Nms, Gln, Asn, Glu, Asp, Dmo and Glu(AGLU);
(e) Xaa5 is a residue of an amino acid wherein Z3 is absent, which is selected from the group consisting of Gly, Nmg, D-ala, D-pro, D-pip, D-aze, Nma and Aib;
(f) Xaa6 is (f1) a residue of an L-α-amino acid selected from the group consisting of Ala, Asp, Asn, Hse, Gln, Glu, Lys, Orn, Dab, N-methyl-Asp, C(Bzl), C(2Quuyl), Eem, Tyr(Bzl) and E(NHMe2Nph), or (f2) is a residue of an L-α-amino acid comprising a functional group FG3 forming a covalent linkage B2 with a functional group of Xaa11, which is selected from the group consisting of Dap, Dab, Orn, Lys, Cys, Hcy, Pen, Asp and Glu;
(g) Xaa9 is a residue of an amino acid selected from the group consisting of Gly, Ala, His, Thr, Dmo and Glu(AGLU);
(h) Xaa11 is one selected from (h1) a residue of Ser, (h2) a residue of an L-α-amino acid comprising the functional group FG2 forming the covalent linkage B1 with the functional group FG1 of Xaa2, which is selected from the group consisting of Dap, Dab, Orn, Lys, Cys, Hcy, Pen, Asp and Glu, and (h3) a residue of an L-α-amino acid comprising the functional group FG4 forming the covalent linkage B2 with the functional group FG3 of Xaa6, which is selected from the group consisting of Dap, Dab, Orn, Lys, Cys, Hcy, Pen, Asp and Glu.

In further preferred embodiments, in the above formulae, all of Xaa1, Xaa2, Xaa4, Xaa5, Xaa6, Xaa9 and Xaa11 are defined as follows while Y is preferably as defined above under item (a):
(b) Xaa1 is absent, or represents a residue of Val;
(c) Xaa2 is (cl) a residue of an L-α-amino acid selected from the group consisting of Tyr, Nmy, Gln, Arg, Dmo and Ser, or (c2) is a residue of an L-α-amino acid comprising a functional group FG1 forming a covalent linkage B1 with a functional group FG2 of Xaa11, which is selected from the group consisting of Dap, Dab, Orn, Lys, Cys, Hcy, Pen, Asp and Glu;
(d) Xaa4 is a residue of an L-α-amino acid selected from the group consisting of Ala, Ser, Glu, Gln and Hse;
(e) Xaa5 is a residue of an amino acid wherein Z3 is absent, which is selected from the group consisting of Gly, Nmg, D-ala, D-pro, D-pip, D-aze, Nma and Aib;
(f) Xaa6 is (f1) a residue of an L-α-amino acid selected from the group consisting of Ala, Asp, Asn, Hse, Gln, Glu, Lys, Orn, Dab, N-methyl-Asp, C(Bzl), C(2Quuyl), Eem, Tyr(Bzl) and E(NHMe2Nph);
(g) Xaa9 is a residue of an amino acid selected from the group consisting of Gly, Ala, His, Thr, Dmo and Glu(AGLU);
(h) Xaa11 is (h1) a residue of Ser, or (h2) a residue of an L-α-amino acid comprising the functional group FG2 forming the covalent linkage B1 with the functional group FG1 of Xaa2, which is selected from the group consisting of Dap, Dab, Orn, Lys, Cys, Hcy, Pen, Asp and Glu.

In further preferred embodiments, in the above formulae, all of Xaa1, Xaa2, Xaa4, Xaa5, Xaa6, Xaa9 and Xaa11 are defined as follows while Y is preferably as defined above under item (a):
(b) Xaa1 is absent;
(c) Xaa2 is (cl) a residue of Gln, or (c2) is a residue of an L-α-amino acid comprising a functional group FG1 forming a covalent linkage B1 with a functional group FG2 of Xaa11, which Xaa2 is Glu;
(d) Xaa4 is a residue of Glu;
(e) Xaa5 is a residue of D-pro;
(f) Xaa6 is a residue of Asp;
(g) Xaa9 is a residue of Thr;
(h) Xaa11 is (h1) a residue of Ser, or (h2) a residue of an L-α-amino acid comprising the functional group FG2 forming the covalent linkage B1 with the functional group FG1 of Xaa2, which Xaa11 is Dap.

In preferred embodiments, the compound of the present invention is selected from the group consisting of:

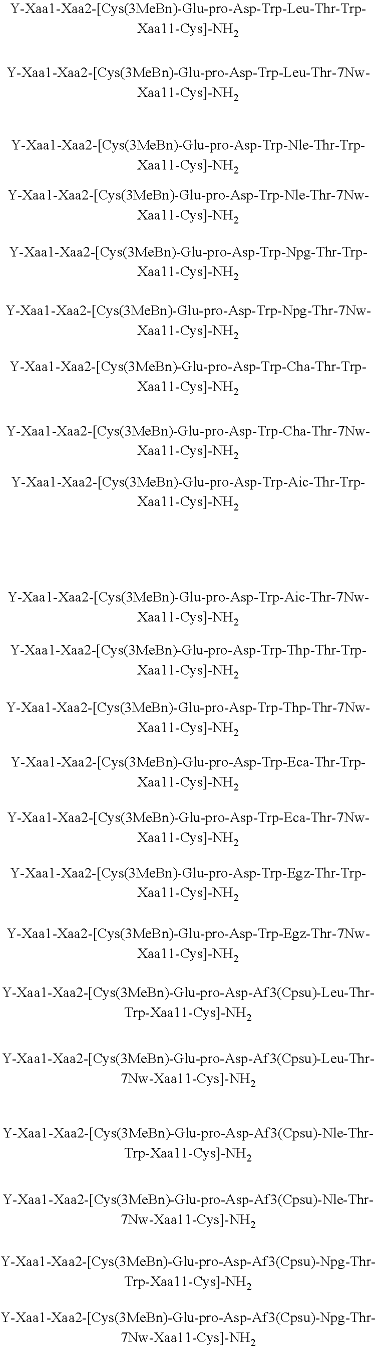

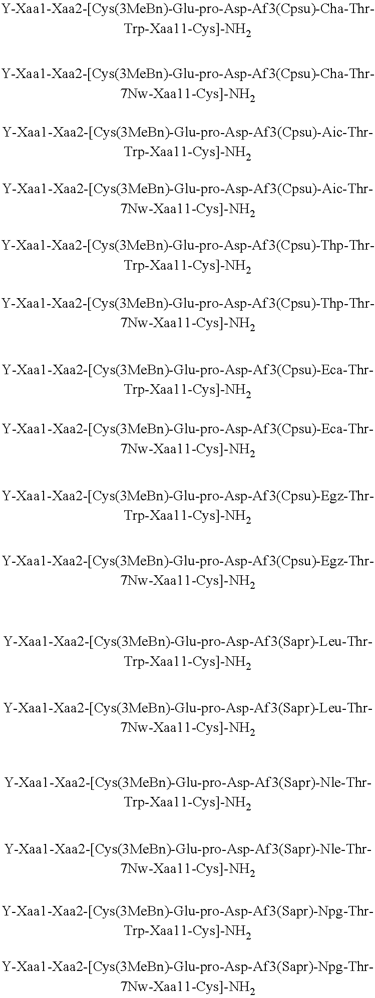

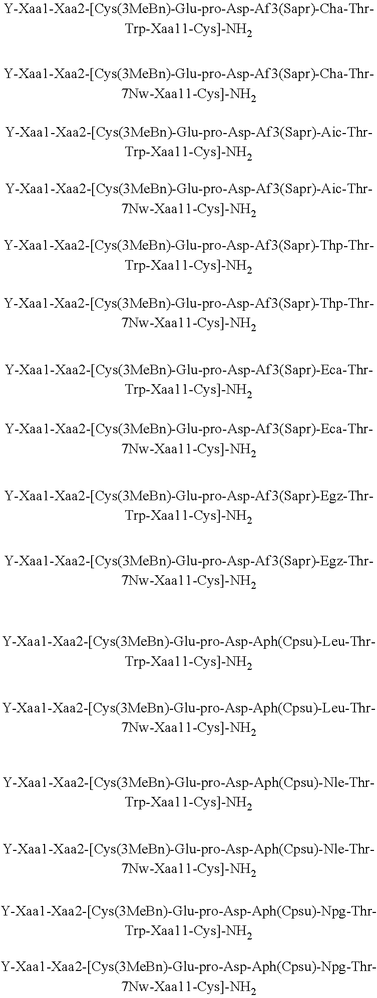

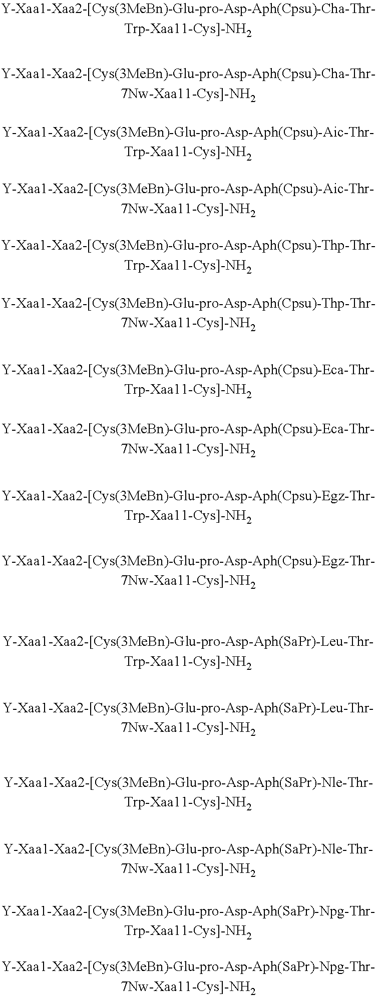

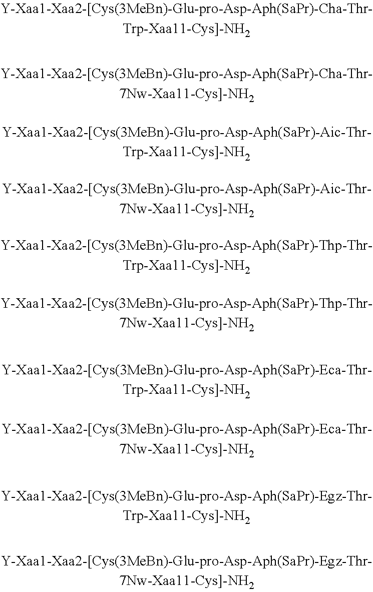

wherein Y, Xaa1, Xaa2 and Xaa11 are as defined above, and wherein preferably at least one—e.g., two, three, or four, of Y, Xaa1, Xaa2 and Xaa11 is/are defined as follows:
(a) Y is a group selected from (a1) Ac, (a2) a moiety comprising an effector E1, and (a3) Z1;
(b) Xaa1 is absent, or represents a residue of an L-α-amino acid selected from the group consisting of Val, Ile, Tle, Ser and Thr;
(c) Xaa2 is (cl) a residue of an L-α-amino acid selected from the group consisting of Tyr, Nmy, Phe, Gln, Arg, Dmo, Ser, Thr, Asp, Glu and Glu(AGLU), or (c2) is a residue of an L-α-amino acid comprising a functional group FG1 forming a covalent linkage B1 with a functional group FG2 of Xaa11, which is selected from the group consisting of Dap, Dab, Orn, Lys, Cys, Hcy, Pen, Asp and Glu;
(h) Xaa11 is one selected from (h1) a residue of Ser, (h2) a residue of an L-α-amino acid comprising the functional group FG2 forming the covalent linkage Bi with the functional group FG1 of Xaa2, which is selected from the group consisting of Dap, Dab, Orn, Lys, Cys, Hcy, Pen, Asp and Glu, and (h3) a residue of an L-α-amino acid comprising the functional group FG4 forming the covalent linkage B2 with the functional group FG3 of Xaa6, which is selected from the group consisting of Dap, Dab, Orn, Lys, Cys, Hcy, Pen, Asp and Glu.

In further preferred embodiments, in the above formulae, at least one—e.g., two or three—of Xaa1, Xaa2 and Xaa11 is/are defined as follows while Y is preferably as defined above under item (a):
(b) Xaa1 is absent, or represents a residue of Val;
(c) Xaa2 is (cl) a residue of an L-α-amino acid selected from the group consisting of Tyr, Nmy, Gln, Arg, Dmo and Ser, or (c2) is a residue of an L-α-amino acid comprising a functional group FG1 forming a covalent linkage B1 with a functional group FG2 of Xaa11, which is selected from the group consisting of Dap, Dab, Orn, Lys, Cys, Hcy, Pen, Asp and Glu;
(h) Xaa11 is (h1) a residue of Ser, or (h2) a residue of an L-α-amino acid comprising the functional group FG2 forming the covalent linkage B1 with the functional group FG1 of Xaa2, which is selected from the group consisting of Dap, Dab, Orn, Lys, Cys, Hcy, Pen, Asp and Glu.

In further preferred embodiments, in the above formulae, at least one—e.g., two or three—of Xaa1, Xaa2 and Xaa11 is/are defined as follows while Y is preferably as defined above under item (a):
(b) Xaa1 is absent;
(c) Xaa2 is (cl) a residue of Gln, or (c2) is a residue of an L-α-amino acid comprising a functional group FG1 forming a covalent linkage B1 with a functional group FG2 of Xaa11, which Xaa2 is Glu;
(h) Xaa11 is (h1) a residue of Ser, or (h2) a residue of an L-α-amino acid comprising the functional group FG2 forming the covalent linkage B1 with the functional group FG1 of Xaa2, which Xaa11 is Dap.

In more preferred embodiments, the compound of the present invention is selected from the group consisting of:

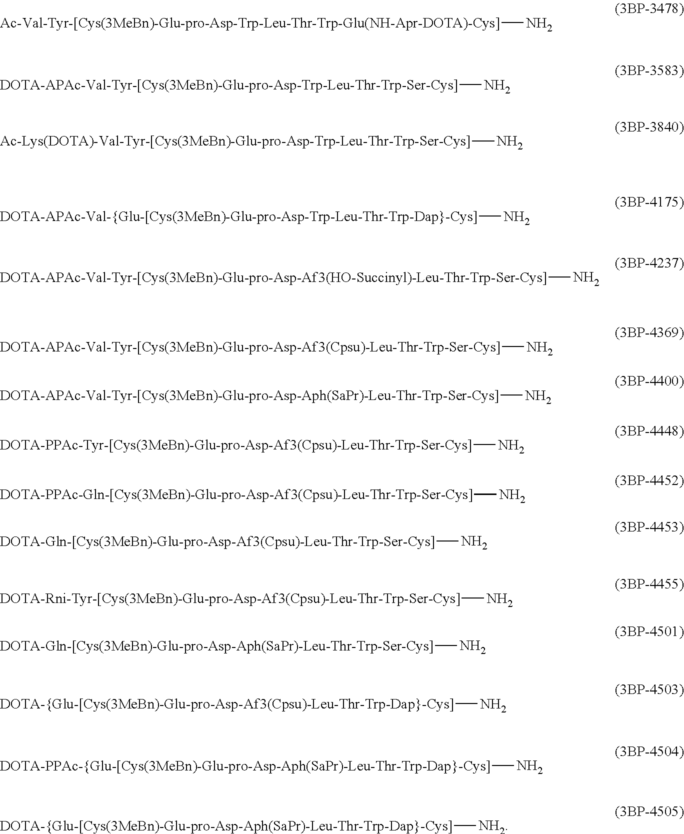

Even more preferably, the compound of the present invention is selected from the group consisting of.

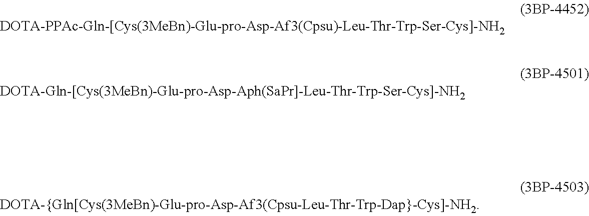

Most preferably, the compound of the present invention is:

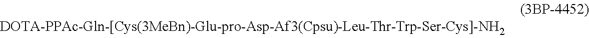

In an embodiment, a compound of the invention is a compound the amino acid sequence of which has an identity of at least 72.7% to an amino acid sequence of a compound of the invention consisting, in terms of amino acid residues, of amino acid residues Xaa2, Xaa3, Xaa4, Xaa5, Xaa6, Xaa7, Xaa8, Xaa9, Xaa10, Xaa11 and Xaa12 (in the following “reference compound of the invention”), wherein Xaa2, Xaa3, Xaa4, Xaa5, Xaa6, Xaa7, Xaa8, Xaa9, Xaa10, Xaa11 and Xaa12 have the preferred meanings according to any one of embodiments (A) and (Ab) described above. Preferably, the amino acid sequence of the reference compound of the invention is selected from the group consisting of Gln-Cys(3MeBn)-Glu-pro-Asp-Af3(Cpsu)-Leu-Thr-Trp-Ser-Cys, Gln-Cys(3MeBn)-Glu-pro-Asp-Aph(SaPr)-Leu-Thr-Trp-Ser-Cys, and Glu-Cys(3MeBn)-Glu-pro-Asp-Af3(Cpsu)-Leu-Thr-Trp-Dap-Cys. Preferably, the identity is at least 81.8% and more preferably the identity is at least 90.9%. It will be appreciated by a person skilled in the art that an identity of 72.7% means that the compound of the invention differs from the reference compound of the invention by 3 amino acid residues, that an identity of 81.8% means that the compound of the invention differs from the reference compound of the invention by 2 amino acid residues, and that an identity of 90.9% means that the compound of the invention differs from the reference compound of the invention by 1 amino acid residue.

In an embodiment, a compound of the invention is a compound the amino acid sequence of which has an identity of at least 75% to an amino acid sequence of a compound of the invention consisting, in terms of amino acid residues, of amino acid residues Xaa1, Xaa2, Xaa3, Xaa4, Xaa5, Xaa6, Xaa7, Xaa8, Xaa9, Xaa10, Xaa11 and Xaa12 (in the following “reference compound of the invention”), wherein the amino acid residues Xaa1, Xaa2, Xaa3, Xaa4, Xaa5, Xaa6, Xaa7, Xaa8, Xaa9, Xaa10, Xaa11 and Xaa12 have the preferred meanings of any one of embodiments (B) and (Bb) described above. Preferably, the amino acid sequence of the reference compound of the invention is selected from the group consisting of Val-Tyr-Cys(3MeBn)-Glu-pro-Asp-Trp-Leu-Thr-Trp-Glu-Cys, Ser-Tyr-Cys(3MeBn)-Glu-pro-Asp-Trp-Leu-Thr-Trp-Glu-Cys, Ile-Tyr-Cys(3MeBn)-Glu-pro-Asp-Trp-Leu-Thr-Trp-Glu-Cys, Thr-Tyr-Cys(3MeBn)-Glu-pro-Asp-Trp-Leu-Thr-Trp-Glu-Cys, Val-Arg-Cys(3MeBn)-Glu-pro-Asp-Trp-Leu-Thr-Trp-Glu-Cys, Val-Phe-Cys(3MeBn)-Glu-pro-Asp-Trp-Leu-Thr-Trp-Glu-Cys, Val-Gln-Cys(3MeBn)-Glu-pro-Asp-Trp-Leu-Thr-Trp-Glu-Cys, and Val-Nmy-Cys(3MeBn)-Glu-pro-Asp-Trp-Leu-Thr-Trp-Glu-Cys. Preferably, the identity is at least 83.3% and more preferably the identity is at least 92.7%. It will be appreciated by a person skilled in the art that an identity of 75% means that the compound of the invention differs from the reference compound of the invention by 3 amino acid residues, that an identity of 83.3% means that the compound of the invention differs from the reference compound of the invention by 2 amino acid residues, and that an identity of 92.7% means that the compound of the invention differs from the reference compound of the invention by 1 amino acid residue.

The identity between two amino acid sequences can be determined as known to the person skilled in the art. More specifically, a sequence comparison algorithm may be used for calculating the percent sequence identity (or homology) for the test sequence(s) relative to the reference sequence, based on the designated program parameters. The test sequence is preferably the amino acid sequence which is said to be identical or to be tested whether it is identical, and if so, to what extent, to a different amino acid sequence such as the amino acid sequence of the reference compound of the invention. Optimal alignment of amino acid sequences can be conducted, e.g., by the local homology algorithm of Smith & Waterman (Smith and Waterman (1981), Adv. Appl. Math. 2: 482) by the homology alignment algorithm of Needleman & Wunsch (Needleman and Wunsch (1970) A general method applicable to the search for similarities in the amino acid sequence of two proteins. J Mol Biol. 48(3):443-53) by the search for similarity method of Pearson & Lipman (Pearson and Lipman (1988) Improved tools for biological sequence comparison. Proc. Nat'l. Acad. Sci. USA 85: 2444), by computerized implementations of these algorithms (GAP, BESTFIT, FASTA, and TFASTA in the Wisconsin Genetics Software Package, Genetics Computer Group, 575 Science Dr., Madison, Wis.), or by visual inspection.

One example of an algorithm that is suitable for determining percent sequence identity is the algorithm used in the basic local alignment search tool (hereinafter “BLAST”), see, e.g. Altschul et al (Altschul S. F., Gish W., et al. (1990) Basic local alignment search tool. J Mol Biol. 215(3):403-10; Altschul S. F., Madden T. L., et al. (1997) Gapped BLAST and PSI-BLAST: a new generation of protein database search programs. Nucleic Acids Res. 25(17):3389-402.). Software for performing BLAST analyses is publicly available through the National Center for Biotechnology Information (hereinafter “NCBI”). The default parameters used in determining sequence identity using the software available from NCBI, e.g., BLASTN (for nucleotide sequences) and BLASTP (for amino acid sequences) are described in McGinnis et al (McGinnis S., Madden T. L. et al. (2004) BLAST: at the core of a powerful and diverse set of sequence analysis tools. Nucleic Acids Res. 32(Web Server issue):W20-5).

### 3. EFFECTOR(S)

In certain embodiments, the compound of the present invention includes one or more “effectors”. As effector we understand a chemical group and/or chemical element attached to the compound or peptide for the purpose of diagnostic and/or therapeutic intervention with CAIX receptor-related diseases/cancer cells. The effector(s) to be used is/are not particularly limited and any effector such as a label and/or pharmaceutically active molecule can be employed.

In preferred embodiments, each effector E1, E3, E4, and E6 is independently selected from the group consisting of:
(α) a moiety derived from a chromophore, wherein the chromophore is preferably selected from (α1) a phosphorophore and (α2) a fluorophore such as fluorescein or rhodamine; and
(β) a chelator optionally comprising a chelated nuclide; and
(γ) a moiety derived from a drug, preferably from a cytotoxic drug.

If the compound of formula (1a) contains more than one effector—e.g., two, three or four effectors—the effectors may be different or identical to each other. Preferably, the effectors are identical to each other. However, it is particularly preferred that the compound of the invention comprises only one effector. It is even more preferred that the effector is comprised by the N-terminal group Y.

In one embodiment, the effector is moiety derived from a chromophore, wherein the chromophore is preferably selected from a phosphorophore and a fluorophore. A fluorophore can be used, e.g., for resection surgery, i.e., operation to remove cancerous tissue wherein the fluorophore is used to make the tumour visible by the fluorescence emitted upon suitable irradiation (“glowing effect”). According to this embodiment, the compound of the present invention preferably does not comprise a chelator in addition to the fluorophore. In these embodiments, the fluorophore may be covalently bound to the cyclic peptide structure by means of linker moieties such as L1, L3, L4, or L6 (as described above).

In one embodiment, the effector is a chelator which comprises a chelated nuclide. The chelator may be covalently bound to the cyclic peptide structure by means of linker moieties such as L1, L3, L4, or L6 (as described above). In the present invention, the linker group forms covalent bonds with both the chelator group and the respective part of the compounds of invention where it is attached. The linker group may, in principle, comprise any chemical group which is capable of forming amide bonds with both the chelator group and the part of the compounds of invention at the specified positions.

In an embodiment, the effector is a chelator which does not comprise a chelated nuclide, i.e. the chelator is a chelator without a chelated nuclide.

The use of linkers usually follows a purpose. In some circumstances it is necessary to space a larger moiety apart from a bioactive molecule in order to retain high bioactivity. In other circumstances introduction of a linker opens the chance to tune physicochemical properties of the molecule by introduction of polarity or multiple charges. In certain circumstances it might be a strength and achievement if one can combine the chelator with a bioactive compound without the need for such linkers.

As preferably used herein, an amino acid is directly linked to the chelator if no linker is interspersed between the amino acid and the chelator.

Preferably, the chelator is part of the compound of the invention, whereby the chelator is either directly or indirectly such as by a linker attached to the compound of the invention. The chelator forms metal chelates preferably comprising at least one radioactive metal. The at least one radioactive metal is preferably useful in or suitable for diagnostic and/or therapeutic and/or theragnostic use and is more preferably useful in or suitable for imaging and/or radiotherapy.

It will be acknowledged by a person skilled in the art that the radioactive nuclide which is or which is to be attached to the compound of the invention, is selected taking into consideration the disease to be treated and/or the disease to be diagnosed, respectively, and/or the particularities of the patient and patient group, respectively, to be treated and to be diagnosed, respectively.

In an embodiment of the present invention, the radioactive nuclide is also referred to as radionuclide. Radioactive decay is the process by which an atomic nucleus of an unstable atom loses energy by emitting ionizing particles (ionizing radiation). There are different types of radioactive decay. A decay, or loss of energy, results when an atom with one type of nucleus, called the parent radionuclide, transforms to an atom with a nucleus in a different state, or to a different nucleus containing different numbers of protons and neutrons. Either of these products is named the daughter nuclide. In some decays the parent and daughter are different chemical elements, and thus the decay process results in nuclear transmutation (creation of an atom of a new element). For example, the radioactive decay can be alpha decay, beta decay, and gamma decay. Alpha decay occurs when the nucleus ejects an alpha particle (helium nucleus). This is the most common process of emitting nucleons, but in rarer types of decays, nuclei can eject protons, or specific nuclei of other elements (in the process called cluster decay). Beta decay occurs when the nucleus emits an electron (β⁻-decay) or positron (β⁺-decay) and a type of neutrino, in a process that changes a proton to a neutron or the other way around. By contrast, there exist radioactive decay processes that do not result in transmutation. The energy of an excited nucleus may be emitted as a gamma ray in gamma decay, or used to eject an orbital electron by interaction with the excited nucleus in a process called internal conversion, or used to absorb an inner atomic electron from the electron shell whereby the change of a nuclear proton to neutron causes the emission of an electron neutrino in a process called electron capture (EC), or may be emitted without changing its number of proton and neutrons in a process called isomeric transition (IT). Another form of radioactive decay, the spontaneous fission (SF), is found only in very heavy chemical elements resulting in a spontaneous breakdown into smaller nuclei and a few isolated nuclear particles.

In a preferred embodiment of the present invention, the radionuclide can be used for labeling of the compound of the invention.

In an embodiment of the present invention, the radionuclide is suitable for complexing with a chelator, leading to a radionuclide chelate complex.

In a further embodiment one or more atoms of the compound of the invention are of non-natural isotopic composition, preferably these atoms are radionuclides; more preferably radionuclides of carbon, oxygen, nitrogen, sulfur, phosphorus and halogens: These radioactive atoms are typically part of amino acids, in some case halogen containing amino acids, and/or building blocks and in some cases halogenated building blocks each of the compound of the invention.

In a preferred embodiment of the present invention, the radionuclide has a half-life that allows for diagnostic and/or therapeutic medical use. Specifically, the half-life is between 1 min and 100 days.

In a preferred embodiment of the present invention, the radionuclide has a decay energy that allows for diagnostic and/or therapeutic medical use. Specifically, for γ-emitting isotopes, the decay energy is between 0.004 and 10 MeV, preferably between 0.05 and 4 MeV, for diagnostic use. For positron-emitting isotopes, the decay energy is between 0.6 and 13 MeV, preferably between 1 and 6 MeV, for diagnostic use. For particle-emitting isotopes, the decay energy is between 0.04 and 10 MeV, preferably between 0.4 and 7 MeV, for therapeutic use.

In a preferred embodiment of the present invention, the radionuclide is industrially produced for medical use. Specifically, the radionuclide is available in GMP quality.

In a preferred embodiment of the present invention, the daughter nuclide(s) after radioactive decay of the radionuclide are compatible with the diagnostic and/or therapeutic medical use.

Furthermore, the daughter nuclides are either stable or further decay in a way that does not interfere with or even support the diagnostic and/or therapeutic medical use. Representative radionuclides which may be used in connection with the present invention are well known to the person skilled in the art and include, but are not limited, to the following ones: ¹¹C, ¹³N, ¹⁸F, ²⁴Na, ²⁸Mg, ³¹Si, ³²P, ³³P, ³⁸Cl, ^{34m}Cl, ³⁸Cl, ³⁹Cl, ³⁷Ar, ⁴¹Ar, ⁴⁴Ar, ⁴²K, ⁴³K, ⁴⁴K, ⁴⁵K, ⁴⁷Ca, ⁴³Sc, ⁴⁴Sc, ^{44m}Sc, ⁴⁷Sc, ⁴⁸Sc, ⁴⁹Sc, ⁴⁵Ti, ⁴⁷V, ⁴⁸V, ⁴⁸Cr, ⁴⁹Cr, ⁵¹Cr, ⁵¹Mn, ⁵²Mn, ^{52m}Mn, ⁵⁶Mn, ⁵²Fe, ⁵⁹Fe, ⁵⁵Co, ⁶¹Co, ^{62m}Co, ⁵⁶Ni, ⁵⁷Ni, ⁶⁵Ni, ⁶⁶Ni, ⁶⁰Cu, ⁶¹Cu, ⁶⁴Cu, ⁶⁷Cu, ⁶²Zn, ⁶³Zn, ⁶⁹Zn, ^{69m}Zn, ^{71m}Zn, ⁷²Zn, ⁶⁵Ga, ⁶⁶Ga, ⁶⁷Ga, ⁶⁸Ga, ⁷⁰Ga, ⁷²Ga, ⁷³Ga, ⁶⁶Ge, ⁶⁷Ge, ⁶⁹Ge, ⁷¹Ge, ⁷⁵Ge, ⁷⁷Ge, ⁷⁸Ge, ⁶⁹As, ⁷⁰As, ⁷¹As, ⁷²As, ⁷⁴As, ⁷⁶As, ⁷⁷As, ⁷⁸As, ⁷⁰Se, ⁷²Se, ⁷³Se, ^{73m}Se, ⁸¹Se, ^{81m}Se, ⁸³Se, ⁷⁴Br, ^{74m}Br, ⁷⁵Br, ⁷⁶Br, ⁷⁷Br, ⁸⁰Br, ^{80m}Br, ⁸²Br, ⁸³Br, ⁸⁴Br, ⁷⁴Kr, ⁷⁶Kr, ⁷⁷Kr, ⁷⁹Kr, ⁸⁵Kr, ⁸⁷Kr, ⁸⁸Kr, ⁷⁸Rb, ⁷⁹Rb, ⁸¹Rb, ⁸²Rb, ⁸⁴Rb, ^{84m}Rb, ⁸⁶Rb, ⁸⁸Rb, ⁸⁹Rb, ⁸⁰Sr, ⁸¹Sr, ⁸²Sr, ⁸³Sr, ^{85m}Sr, ⁸⁷Sr, ⁹¹Sr, ⁹²Sr, ⁸⁴Y, ⁸⁵Y, ^{85m}Y, ⁸⁶Y, ^{86m}Y, ⁸⁷Y, ^{87m}Y, ⁹⁰Y, ^{90m}Y, ^{91m}Y, ⁹²Y, ⁹³Y, ⁹⁴Y, ⁹⁵Y, ⁸⁶Zr, ⁸⁷Zr, ⁸⁹Zr, ⁹⁷Zr, ⁸⁸Nb, ⁸⁹Nb, ^{89m}Nb, ⁹⁰Nb, ⁹²Nb, ⁹⁵Nb, ^{95m}Nb, ⁹⁶Nb, ⁹⁷Nb, ^{98m}Nb, ¹⁰¹Mo, ¹⁰²Mo, ⁹⁰Mo, ⁹¹Mo, ^{93m}Mo, ⁹⁹Mo, ¹⁰¹Tc, ¹⁰⁴Tc, ⁹³Tc, ^{93m}Tc, ⁹⁴Tc, ^{94m}Tc, ⁹⁵Tc, ⁹% Tc, ^{99m}Tc, ¹⁰³Ru, ¹⁰⁵Ru, ⁹⁴Ru, ⁹⁵Ru, ⁹⁷Ru, ¹⁰⁰Rh, ^{101m}Rh, ¹⁰⁵Rh, ^{106m}Rh, ¹⁰⁷Rh, ⁹⁷Rh, ^{97m}Rh, ⁹⁹Rh, ^{99m}Rh, ¹⁰⁰Pd, ¹⁰¹Pd, ¹⁰³Pd, ¹⁰⁹Pd, ¹¹¹Pd, ^{111m}pd, ¹¹²Pd, ⁹⁸Pd, ⁹⁹Pd, ¹⁰¹Ag, ¹⁰³Ag, ¹⁰⁴Ag, ^{104m} Ag, ¹⁰⁵Ag, ¹⁰⁶Ag, ^{106m} Ag, ¹¹¹Ag, ¹¹²Ag, ¹¹³Ag, ¹¹⁵Ag, ¹⁰⁴Cd, ¹⁰⁵Cd, ¹⁰⁷Cd, ¹¹¹Cd, ¹¹⁵Cd, ^{115m}Cd, ¹¹⁷Cd, ^{117m}Cd, ¹¹⁸Cd, ¹⁰⁷In, ^{108m} In, ¹⁰⁹In, ¹¹⁰In, ^{110m} In, ¹¹¹In, ¹¹²In, ¹¹³In, ^{114m} In, ^{115m}In, ^{116m}In, ¹¹⁷In, ^{117m} In, ^{119m}In, ¹⁰⁸Sn, ¹⁰⁹Sn, ¹¹⁰Sn, ¹¹¹Sn, ¹¹⁷Sn, ¹²¹Sn, ^{123m}Sn, ¹²⁵Sn, ¹²⁷Sn, ¹²⁸Sn, ¹¹⁵Sb, ¹¹⁶Sb, ^{116m}Sb, ¹¹⁷Sb, ^{118m}Sb, ¹¹⁹Sb, ¹²⁰Sb, ^{120m}Sb, ¹²²Sb, ¹²⁶Sb, ^{126m}Sb, ¹²⁷Sb, ⁸Sb, ^{128m}Sb, ¹²⁹Sb, ^{129m}Sb, ¹³⁰Sb, ¹³¹Sb, ¹¹⁴Te, ¹¹⁶Te, ¹¹⁷Te, ¹¹⁸Te, ¹¹⁹Te, ^{119m}Te, ¹²¹Te, ¹²⁷Te, ¹²⁹Te, ^{129m}Te, ¹³¹Te, ^{131m}Te, ¹³²Te, ¹³³Te, ^{133m}Te, ¹³⁴Te, ¹¹⁸I, ¹¹⁹I, ¹²⁰I, ^{120m}I, ¹²¹I, ¹²³I, ¹²⁴I, ¹²⁶I, ¹²⁸I, ¹³⁰I, ¹³¹I, ¹³²I, ^{132m}I, ¹³³I, ¹³⁴I, ¹³⁵I, ¹²⁰Xe, ¹²¹Xe, ¹²²Xe, ¹²³Xe, ¹²⁵Xe, ¹²⁷Xe, ¹³³Xe, ^{133m} Xe, ¹³⁵Xe, ^{135m} Xe, ¹³⁸Xe, ¹²⁵Cs, ¹²⁷Cs, ¹²⁹Cs, ¹³⁰Cs, ¹³¹Cs, ¹³²Cs, ¹³⁴Cs, ¹²⁴Ba, ¹²⁶Ba, ¹²⁷Ba, ¹²⁸Ba, ¹²⁹Ba, ^{129m}Ba, ¹³¹Ba, ^{131m}Ba, ¹³³Ba, ¹³⁵Ba, ¹³⁹Ba, ¹⁴⁰Ba, ¹⁴¹Ba, ¹⁴²Ba, ¹²⁹La, ¹³¹La, ¹³²La, ¹³³La, ¹³⁵La, ¹⁴⁰La, ¹⁴¹La, ¹⁴²La, ¹⁴³La, ¹³⁰Ce, ¹³²Ce, ¹³³Ce, ^{133m}Ce, ¹³⁴Ce, ¹³⁵Ce, ¹³⁷Ce, ^{137m}Ce, ¹⁴¹Ce, ¹⁴³Ce, ¹⁴⁶Ce, ¹³⁴Pr, ^{134m}Pr, ¹³⁶Pr, ¹³⁷Pr, ^{138m}Pr, ¹³⁹Pr, ¹⁴²Pr, ¹⁴³Pr, ¹⁴⁴Pr, ¹⁴⁵Pr, ¹⁴⁶Pr, ¹⁴⁷Pr, ¹³⁵Nd, ¹³⁶Nd, ¹³⁷Nd, ¹³⁸Nd, ¹³⁹Nd, ^{139m}Nd, ¹⁴⁰Nd, ¹⁴¹Nd, ¹⁴⁷Nd, ¹⁴⁹Nd, ¹⁵¹Nd, ¹⁵²Nd, ¹⁴¹Pm, ¹⁴⁸Pm, ^{148m}Pm, ¹⁴⁹Pm, ¹⁵⁰Pm, ¹⁵¹Pm, ¹⁴⁰Sm, ¹⁴¹Sm, ^{141m}Sm, ¹⁴²Sm, ¹⁵³Sm, ¹⁵⁵Sm, ¹⁵⁶Sm, ¹⁴⁵Eu, ¹⁴⁶Eu, ¹⁴⁷Eu, ¹⁵⁰Eu, ^{152m}Eu, ¹⁵⁴Eu, ¹⁵⁶Eu, ¹⁵⁷Eu, ¹⁵⁸Eu, ¹⁵⁹Eu, ¹⁴⁵Gd, ¹⁴⁶Gd, ¹⁴⁷Gd, ¹⁴⁹Gd, ¹⁵⁹Gd, ¹⁴⁷Tb, ¹⁴⁸Tb, ¹⁴⁹Tb, ¹⁵⁰Tb, ¹⁵¹Tb, ¹⁵²Tb, ¹⁵³Tb, ¹⁵⁴Tb, ^{154m}Tb, ¹⁵⁵Tb, ¹⁵⁶Tb, ^{156m}Tb, ¹⁶¹Tb, ¹⁶³Tb, ¹⁵¹Dy, ¹⁵²Dy, ¹⁵³Dy, ¹⁵⁵Dy, ¹⁵⁷Dy, ¹⁶⁵Dy, ¹⁶⁶Dy, ¹⁵⁴Ho, ¹⁵⁵Ho, ¹⁵⁶Ho, ¹⁵⁷Ho, ^{158m}Ho, ¹⁵⁹Ho, ¹⁶¹Ho, ¹⁶²Ho, ^{162m}Ho, ¹⁶⁴Ho, ^{164m}Ho, ¹⁶⁶Ho, ¹⁶⁷Ho, ¹⁵⁶Er, ¹⁵⁷Er, ¹⁵⁸Er, ¹⁵⁹Er, ¹⁶⁰Er, ¹⁶¹Er, ¹⁶³Er, ¹⁶⁵Er, ¹⁶⁹Er, ¹⁷¹Er, ¹⁷²Er, ¹⁶¹Tm, ¹⁶²Tm, ¹⁶³Tm, ¹⁶⁵Tm, ¹⁶⁶Tm, ¹⁶⁷Tm, ¹⁷²Tm, ¹⁷³Tm, ¹⁷⁵Tm, ¹⁶²Yb, ¹⁶³Yb, ¹⁶⁴Yb, ¹⁶⁶Yb, ¹⁶⁷Yb, ¹⁶⁹Yb, ¹⁷⁵Yb, ¹⁷⁷Yb, ¹⁷⁸Yb, ¹⁶⁷Lu, ¹⁶⁹Lu, ¹⁷⁰Lu, ¹⁷¹Lu, ¹⁷²Lu, ^{176m}Lu, ¹⁷⁷Lu, ¹⁷⁸Lu, ^{178m}Lu, ¹⁷⁹Lu, ¹⁶⁸Hf, ¹⁷⁰Hf, ¹⁷³Hf, ^{177m}Hf, ^{179m}Hf, ^{180m}Hf, ¹⁸¹Hf ^{182m}Hf, ¹⁸³Hf, ¹⁸⁴Hf, ¹⁷²Ta, ¹⁷³Ta, ¹⁷⁴Ta, ¹⁷⁵Ta, ¹⁷⁶Ta, ¹⁷⁷Ta, ¹⁷⁸Ta, ¹⁸⁰Ta, ^{182m}Ta, ¹⁸⁶Ta, ¹⁷⁴W, ¹⁷⁵W, ¹⁸³Ta, ¹⁸⁴Ta, ¹⁸⁵Ta, ¹⁷⁷W, ¹⁷⁸W, ¹⁷⁹W, ¹⁸⁷W, ¹⁹⁰W, ¹⁷⁷Re, ¹⁷⁸Re, ⁷⁹Re, ¹⁸¹Re, ¹⁸²Re, ^{182m}Re, ¹⁸⁴Re, ¹⁸⁸Re, ¹⁸⁶Re, ¹⁸⁸Re, ^{188m}Re, ¹⁸⁹Re, ^{190m}Re, ¹⁸⁰Os, ¹⁸¹Os, ¹⁸²Os, ¹⁸³Os, ^{183m}Os, ¹⁹³Os, ¹⁹⁶Os, ¹⁹¹Os, ¹⁸²Ir, ¹⁸³Ir, ¹⁸⁴Ir, ¹⁸⁵Ir, ¹⁸⁶Ir, ^{186m} Ir, ¹⁸⁷Ir, ¹⁸⁸I r, ¹⁸⁹Ir, ¹⁹⁰Ir, ^{195m}Ir, ¹⁹⁵Ir, ^{196m}Ir, ¹⁸⁴Pt, ¹⁸⁶Pt, ¹⁸⁷Pt, ¹⁸⁸Pt, ¹⁸⁹Pt, ¹⁹¹Pt, ¹⁹⁵Pt, ¹⁹⁷Pt, ^{197m}pt, ¹⁹⁹Pt, ²⁰⁰Pt, ²⁰²Pt, ¹⁸⁶Au, ¹⁹⁰Au, ¹⁹¹Au, ¹⁹²Au, ¹⁹³Au, ¹⁹⁴Au, ¹⁹⁶Au, ^{198m} Au, ¹⁹⁹Au, ²⁰⁰Au, ^{200m} Au, ¹⁹⁰Hg, ¹⁹¹Hg, ¹⁹²Hg, ¹⁹³Hg, ¹⁹⁵Hg, ^{195m}Hg, ¹⁹⁷Hg, ^{196m} Au, ¹⁹⁸Au, ^{197m}Hg, ¹⁹⁹Hg, ²⁰³Hg, ¹⁹⁴Tl, ^{194m}Tl, ¹⁹⁵Tl, ¹⁹⁶Tl, ^{196m}Tl, ¹⁹⁷Tl, ¹⁹⁸Tl, ^{198m}Tl, ¹⁹⁹Tl, ²⁰⁰Tl, ²⁰¹Tl, ²⁰²Tl, ¹⁹⁴Pb, ¹⁹⁵Pb, ¹⁹⁶Pb, ^{197m}Pb, ¹⁹⁸Pb, ¹⁹⁹Pb, ^{199m}Pb, ²⁰⁰Pb, ²⁰¹Pb, ^{202m}Pb, ²⁰³Pb, ²⁰⁴Pb, ²⁰⁹Pb, ²¹¹Pb, ²¹²Pb, ²¹⁴Pb, ²⁰⁰Bi, ^{200m}Bi, ²⁰¹Bi, ²⁰²Bi, ²⁰³Bi, ²⁰⁴Bi, ²⁰⁵Bi, ²⁰⁶Bi, ²¹⁰Bi, ²¹²Bi, ^{212m}Bi, ²¹³Bi, ²¹⁴Bi, ²⁰⁰Po, ²⁰¹Po, ²⁰²Po, ²⁰³Po, ²⁰⁴Po, ²⁰⁵Po, ²⁰⁶Po, ²⁰⁷Po, ²⁰⁵At, ²⁰⁶At, ²⁰⁷At, ²⁰⁸At, ²⁰⁹At, ²¹⁰At, ²¹¹At, ²⁰⁸Rn, ²⁰⁹Rn, ²¹⁰Rn, ²¹¹Rn, ²¹²Rn, ²²¹Rn, ²²²Rn, ²²³Rn, ²¹²Fr, ²²²Fr, ²²³Ra, ²²⁴Ra, ²²⁵Ra, ²²⁷Ra, ²²⁴Ac, ²³⁰Ra, ²²⁵Ac, ²²⁶Ac, ²²⁸Ac, ²²⁹Ac, ²²⁶Th, ²²⁷Th, ²³¹Th, ²³³Th, ²³⁴Th, ²³⁶Th, ²²⁷Pa, ²²⁸Pa, ²²⁹Pa, ²³⁰Pa, ²³²Pa, ²³³Pa, ²³⁴Pa, ²³⁵Pa, ²²⁹U, ²³⁰U, ²³¹U, ²³⁷U, ²³⁹U, ²⁴⁰U, ²⁴²U, ²³¹Np, ²³²Np, ²³³Np, ²³⁴Np, ^{236m}Np, ²³⁸Np, ²³⁹Np, ²⁴⁰Np, ²⁴¹Np, ²³²Pu, ²³⁵Pu, ²³⁷Pu, ²⁴³Pu, ²⁴⁵Pu, ²⁴⁶Pu, ²³⁵Am, ²³⁷Am, ²³⁸Am, ²³⁹Am, ²⁴⁰Am, ²⁴²Am, ²⁴⁴Am, ^{244m} Am, ²⁴⁵Am, ²⁴⁶Am, ^{246m} Am, ²⁴⁷Am, ²³⁹Cm, ²⁴⁰Cm, ²⁴¹Cm, ²⁵¹Cm, ²⁴⁵Bk, ²⁴⁶Bk, ²⁴⁸Bk, ²⁵⁰Bk, ²⁵¹Bk, ²⁴⁴Cf, ²⁴⁵Cf, ²⁴⁶Cf, ²⁴⁷Cf, ²⁵³Cf, ²⁵⁵Cf, ²⁴⁹Es, ²⁵⁰Es, ^{250m}Es, ²⁵¹Es, ²⁵³Es, ^{254m}Es, ²⁵⁵Es, ^{256m}Es, ²⁵⁰Fm, ²⁵¹Fm, ²⁵²Fm, ²⁵⁴Fm, ²⁵⁵Fm, ²⁵⁵Md, ²⁵⁶Md, ²⁵⁷Md, ²⁵⁹No, Their properties are described in more detail, for instance, in Nuclear Data Sheets (Elsevier, Amsterdam, NL).

In an embodiment of the present invention, the radionuclide is used for diagnosis. Preferably, the radioactive isotope is selected from the group, but not limited to, comprising ⁴³Sc, ⁴⁴Sc, ⁵¹Mn, ⁵²Mn, ⁶⁴Cu, ⁶⁷Ga, ⁶⁸Ga, ⁸⁶Y, ⁸⁹Zr, ^{94m}Tc, ^{99m}Tc, ¹¹¹In, ¹⁵²Tb, ¹⁵⁵Tb, ¹⁷⁷Lu, ²⁰¹Tl, ²⁰³Pb, ¹⁸F, ⁷⁶Br, ⁷⁷Br, ¹⁴⁹Tb, ¹²³I, ¹²⁴I, and ¹²⁵I. More preferably, the radionuclide is selected from the group comprising ⁴³Sc, ⁴⁴Sc, ⁶⁴Cu, ⁶⁷Ga ⁶⁸Ga, ⁸⁶Y, ⁸⁹Zr, ¹¹¹In, ¹⁵²Tb, ¹⁵⁵Tb, and ²⁰³Pb. Even more preferably, the radionuclide is ⁶⁴Cu, ⁶⁸Ga, ¹¹In, and ²⁰³Pb. It will, however, also be acknowledged by a person skilled in the art that the use of said radionuclide is not limited to diagnostic purposes, but encompasses their use in therapy and theragnostics when conjugated to the compound of the invention.

In an embodiment of the present invention, the radionuclide is used for therapy. Preferably, the radioactive isotope is selected from the group comprising ⁴⁷Sc, ⁶⁷Cu, ⁸⁹Sr, ⁹⁰Y, ¹¹¹In ¹⁵³Sm, ¹⁴⁹Tb, ¹⁶¹Tb ¹⁷⁷Lu, ¹⁸⁶Re, ¹⁸⁸Re, ²¹²Pb, ²¹³Bi, ²²³Ra, ²²⁵Ac, ²²⁶Th, ²²⁷Th, ¹³¹I, and ²¹¹At.

More preferably, the radioactive isotope is selected from the group comprising ⁴⁷Sc, ⁶⁷Cu, ⁹⁰Y, ¹⁷⁷Lu, ²¹²Pb, ²¹³Bi, ²²⁵Ac, and ²²⁷Th. Even more preferably, the radionuclide is selected from the group comprising ⁹⁰Y, ¹⁷⁷Lu, ²¹²Pb, ²²⁵Ac, and ²²⁷Th. It will, however, also be acknowledged by a person skilled in the art that the use of said radionuclide is not limited to therapeutic purposes, but encompasses their use in diagnostic and theragnostics when conjugated to the compound of the invention.

Chelators in principle useful in and/or suitable for the practicing of the instant invention including diagnosis and/or therapy of a disease are known to the person skilled in the art. A wide variety of respective chelators is available and has been reviewed, e.g. by Banerjee et al. (Banerjee, et al., Dalton Trans, 2005, 24: 3886), and references therein (Price, et al., Chem Soc Rev, 2014, 43: 260; Wadas, et al., Chem Rev, 2010, 110: 2858). Such chelators include, but are not limited to linear, cyclic, macrocyclic, tetrapyridine, N3S, N2S2 and N₄ chelators as disclosed in U.S. Pat. Nos. 5,367,080 A, 5,364,613 A, 5,021,556 A, 5,075,099 A and 5,886,142 A.

Representative chelators and their derivatives include, but are not limited to AAZTA, BAT, CDTA, DTA, DTPA, CY-DTA, DTCBP, CTA, cyclam, cyclen, TETA, sarcophagine, CPTA, TEAMA, DO3A, DO2A, TRITA, DATA, DFO, DATA(M), DATA(P), DATA(Ph), DATA(PPh), DEDPA, H4octapa, H2dedpa, H5decapa, H2azapa, H2CHX-DEDPA, DFO-Chx-MAL, DFO-p-SCN, DFO-1AC, DFO-BAC, p-SCN-Bn-DFO, DFO-pPhe-NCS, DFO-HOPO, DFC, diphosphine, DOTA, DOTAGA, DOTA-MFCO, DOTAM-mono-acid, nitro-DOTA, nitro-PA-DOTA, p-NCS-Bz-DOTA, PA-DOTA, DOTA-NCS, DOTA-NHS, CB-DO2A, PCTA, p-NH₂-Bn-PCTA, p-SCN-Bn-PCTA, p-SCN-Bn-DOTA, DOTMA, NB-DOTA, H4NB-DOTA, H4TCE-DOTA, HOPO, 2,3-HOPO, 3,4,3-(L1-1,2-HOPO), TREN(Me-3,2-HOPO), TCE-DOTA, DOTP, DOXP, p-NCS-DOTA, p-NCS-TRITA, TRITA, TETA, 3p-C-DEPA, 3p-C-DEPA-NCS, p-NH₂-BN-OXO-DO3A, p-SCN-BN-TCMC, TCMC, 4-aminobutyl-DOTA, azido-mono-amide-DOTA, BCN-DOTA, butyne-DOTA, BCN-DOTA-GA, DOA3P, DO2a2p, DO2A(trans-H2do2a), H2DO2A, H2ODO2A, DO3A, DO3A-thiol, DO3AM-acetic acid, DO2AP, CB-DO2A, C₃B-DO2A, HP-DO3A, DOTA-NHS-ester, maleimide-DOTA-GA, maleimido-mono-aminde-DOTA, maleimide-DOTA, NH₂-DOTA-GA, NH₂-PEG4-DOTA-GA, GA, p-NH₂-Bn-DOTA, p-N₀₂-Bn-DOTA, p-SCN-Bn-DOTA, p-SCN-Bz-DOTA, TA-DOTA, TA-DOTA-GA, OTTA, DOXP, TSC, DTC, DTCBP, PTSM, ATSM, H2ATSM, H2PTSM, Dp44mT, DpC, Bp44mT, QT, hybrid thiosemicarbazone-benzothiazole, thiosemicarbazone-styrylpyridine tetradentate ligands H2L²⁻⁴, HBED, HBED-CC, dmHBED, dmEHPG, HBED-nn, SHBED, Br-Me2HBED, BPCA, HEHA, BF-HEHA, deferiprone, THP, HYNIC (2-hydrazino nicotinamide), NHS-HYNIC, HYNIC-Kp-DPPB, HYNIC-Ko-DPPB, (HYNIC)(tricine)2, (HYNIC)(EDDA)Cl, p-EDDHA, AIM, AIM A, IAM B, MAMA, MAMA-DGal, MAMA-MGal, MAMA-DA, MAMA-HAD, macropa, macropaquin, macroquin-SO₃, NxS4-x, N2S2, N3S, N4, MAG3B, NOTA, NODAGA, SCN-Bz-NOTA-R, NOT-P (NOTMP), MA-NOTMP, NOTAM, p-NCS-NOTA, TACN, TACN-TM, NETA, NETA-monoamine, p-SCN-PhPr-NE3TA, C-NE3TA-NCS, C-NETA-NCS, 3p-C-NETA, NODASA, NOPO, NODA, NO₂A, N-benzyl-NODA, C-NOTA, BCNOT-monoamine, maleimido-mono-amide-NOTA, NO₂A-azide, NO₂A-butyne, NO₂AP, NO3AP, N-NOTA, oxo-DO3A, p-NH₂-Bn-NOTA, p-NFH-Bn-oxo-DO3A, p-NO2-Bn-cyclen, p-SCN-Bn-NOTA, p-SCN-Bn-oxo-DO3A, TRAP, PEPA, BF-PEPA, pycup, pycup2A, pycuplAlBn, pycup2Bn, SarAr-R, DiAmSar, AmBaSar-R, siamSar, Sar, Tachpyr, tachpyr-(6-Me), TAM A, TAM B, TAME, TAME-Hex, THP-Ph-NCS, THP-NCS, THP-TATE, NTP, H3THP, THPN, CB-TE2A, PCB-TE1A1P, TETA-NHS, CPTA, CPTA-NHS, CB-TE1K1P, CB-TE2A, TE2A, H2CB-TE2A, TE2P, CB-TE2P, MM-TE2A, DM-TE2A, 2C-TETA, 6C-TETA, BAT, BAT-6, NHS-BAT ester, SSBAT, SCN-CHX-A-DTPA-P, SCN-TETA, TMT-amine, p-BZ-HTCPP, DCMC, DEPA, H2ATSM, PCBA, PIH, wherein
2,3-HOPO stands for 3-hydroxypyridin-2-one, 2C-TETA stands for [4,8,11-tris-carboxymethyl-12-(4-isothiocyanato-benzyl)-1,4,8,11tetraaza-cyclotetradec-1-yl]-acetic acid,
3p-C-DEPA stands for 2-[(carboxymethyl)][5-(4-nitrophenyl-1-[4,7,10-tris(carboxymethyl)-1,4,7,10-tetraazacyclododecan-1-yl]pentan-2-yl)amino]acetic acid,
3p-C-DEPA-NCS stands for 2-[(carboxymethyl)][5-(4-thiocyanatophenyl-1-[4,7,10-tris(carboxymethyl)-1,4,7,10-tetraazacyclododecan-1-yl]pentan-2-yl)amino]acetic acid,
3p-C-NE3TA-NCS stands for {4-[2-(bis-carboxymethylamino)-5-(4-isothiocyanatophenyl) pentyl]-7-carboxymethyl[1,4,7]triazonan-1-yl}acetic acid,
3p-C-NETA stands for {4-[2-(bis-carboxymethylamino)-5-(4-nitrophenyl) pentyl]-7-carboxymethyl[1,4,7]triazonan-1-yl}acetic acid, 4-aminobutyl-DOTA stands for 1,4,7,10-tetraazacyclododecane-1,4,7-tris(acetic acid)-10-(4-aminobutyl)acetamide,
^{99m}Tc(CO)₃-chelators stands for bi- or tridendate chelators capable of forming stable complexes with technetium tricarbonyl fragments,
AAZTA stands for 6-amino-6-methylperhydro-1,4-diazepine-N,N′,N″,N″-tetraacetic acid, AmBaSar stands for 4-((8-amino-3,6,10,13,16,19-hexaazabicyclo [6.6.6] icosane-1-ylamino) methyl) benzoic acid,
ATSM stands for diacetyl-bis(N₄-methylthiosemicarbazone), azido-mono-amide-DOTA stands for 1,4,7,10-tetraazacyclododecane-1,4,7-tris(acetic acid)-10-(azidopropyl ethylacetamide),
BAT stands for 3,15,27-triamino-7,19,31-trihydroxy-10,22,34-trimethyl-1,13,25-trioxa-7,19,31-triaza-cyclohexatriaconta-9,21,33-triene-2,8,14,20,26,32-hexaone,
BCN-DOTA-GA stands for 2,2′,2″-(10-(4-((2-((((1R,8S,9s)-bicyclo[6.1.0]non-4-yn-9-ylmethoxy)carbonyl)amino)ethyl)amino)-1-carboxy-4-oxobutyl)-1,4,7,10-tetraazacyclododecane-1,4,7-triyl)triacetic acid,
BF-HEHA stands for 3-(4-isothicyanatobenzyl)-1,2,7,10,13-hexaazacyclooctadecane-1,4,7,10,13,16-hexaacetic acid,
BF-PEPA stands for 2-(4-thiocyanatobenzoyl)-1, 4, 7, 10, 13-pentaazacyclopentadecane-N, N′, N″, N′″, N″″-pentaacetic acid,
Bp44mT stands for 2-benzoylpyridine-4,4-dimethyl-3-thiosemicarbazone,
BPCA stands for bipyridine-chelator,
Br-Me2HBED stands for N-(2-hydroxy-3,5-dimethylbenzyl)-Ar′-(2-hydroxy-5-(bromoacetamido)benzyl)ethylenediamine-N,-N′-diacetic acid,
butyne-DOTA stands for 1,4,7,10-tetraazacyclododecane-1,4,7-tris(acetic acid)-10-(3-butynylacetamide),
CB-DO2A stands for 4,10-bis(carboxymethyl)-1,4,7,10-tetraazabicyclo[5.5.2]tetradecane,
CB-TE1A1P stands for (1,8-diamino-3,6,10,13,16,19-hexaazabicyclo[6.6.6]icosane),
CB-TE1K1P stands for 6-amino-2-(11-phosphonomethyl-1,4,8,11-tetraaza-bicyclo[6.6.2]hexadec-4-yl)-hexanoic acid,
CB-TE2A stands for 4,11-bis-(carboxymethyl)-1,4,8,11-tetraazabicyclo[6.6.2]-hexadecane,
CB-TE2P stands for 1,4,8,11-tetraazacyclotetradecane-1,8-di(methanephosphonic acid), CDTA stands for trans-1,2-diaminocyclohexane-N,N,N′,N′-tetraacetic acid,
CHX-A″-DTPA stands for [(2-{[2-(bis-carboxymethyl-amino)-cyclohexyl]-carboxymethyl-amino}-ethyl)-carboxymethyl-amino]-acetic acid,
C-NOTA stands for [4,7-bis-carboxymethyl-2-(4-nitro-benzyl)-[1,4,7]triazonan-1-yl]-acetic acid,
CPTA stands for 4-((1,4,8,11-tetraazacyclotetradecan-1-yl)methyl)benzoic acid,
cyclam stands for 1,4,8,11-tetraazacyclotetradecane,
cyclen stands for 1,4,7,10-tetraazacyclododecane,
CY-DTA stands for trans-1,2-diaminocyclohexane-N,N,N′,N′-tetraacetic acid, monohydrate,
DATA stands for [4-carboxymethyl-6-(carboxymethyl-methyl-amino)-6-methyl-[1,4]diazepan-1-yl]-acetic acid,
DCMC stands for 1,7-bis(carbamoylmethyl)-1,4,7,10-tetraazacyclodocane,
deferiprone (also called DMHP, CP20, L1) stands for 3-hydroxy-1,2-dimethyl-4(1H)-pyridone,
DEPA stands for 7-[2-(bis-carboxymethylamino)-ethyl]-4,10-bis-carboxymethyl-1,4,7,10-tetraaza-cyclododec-1-yl-acetic acid,
DEPA stands for diethylenetriamine pentaacetic acid,
DFO stands for the Desferal or Desferrioxamine type group of chelators, the chemical name of the non-limiting example is N-[5-({3-[5-(Acetyl-hydroxy-amino)-pentylcarbamoyl]-propionyl}-hydroxy-amino)-pentyl]-N′-(5-amino-pentyl)-N′-hydroxy-succinamide,
DFO-BAC stands for bromoacetyl-desferrioxamine,
DFO-HOPO stands for N1-hydroxy-N1-(5-(4-(hydroxy(5-(1-hydroxy-6-oxo-1,6-dihydropyridine-2-carboxamido)pentyl)amino)-4-oxobutanamido)pentyl)-N4-(5-(Nhydroxyacetamido)pentyl)succinamide,
DFO-pPhe-NCS(=p-SCN-Bn-DFO) stands for 1-(4-isothiocyanatophenyl)-3-[6,17-dihydroxy-7,10,18,21-tetraoxo-27-(N-acetylhydroxylamino)-6,11,17, 22-tetraazaheptaeicosine]thiourea,
DiAmSar stands for 1,8-diamino-3,6,10,13,16,19-hexaazabicyclo[6.6.6]icosane,
dmEHPG stands for N,N-8-ethylene-bis(o-hydroxyphenylglycine) dimethyl ester,
dmHBED stands for N,N*-bis(o-hydroxybenzyl) ethylenediamine diacetic acid,
DM-TE2A stands for 1,8-N,N′-bis-(carboxymethyl)-4,11-N″,N′″-bis-(methyl)-1,4,8,11-tetraazacyclotetra decane,
DO2A stands for 1,4,7,10-tetraazacyclododecane-1,7-diacetic acid,
DO2AP stands for 4-[phosphorylmethyl]-1,4,7,10-tetrazacyclododecane-1,7-diacetic acid,
DO2a2p stands for 1,4,7,10-tetraazacyclododecane-1,7-bis(acetic acid)-4,10-bis(methylenephosphonic acid),
DO3A stands for 1,4,7,10-tetraazacyclododecane-1,4,7-triacetic acid,
DO3AM-acetic acid stands for 2-(4,7,10-tris(2-amino-2-oxoethyl)-1,4,7,10-tetraazacyclododecan-1-yl)acetic acid,
DO3AP stands for 7-[phosphorylmethyl]-1,4,7,10-tetrazacyclododecane-1,4,10-triacetic acid,
DO3A-thiol stands for 1,4,7,10-tetraazacyclododecane-1,4,7-tris(acetic acid)-10-(2-thioethyl)acetamide,
DOTA (also called tetraxetan) stands for 1,4,7,10-tetrazacyclododecane-1,4,7,10-tetraacetic acid,
DOTAGA stands for 1,4,7,10-tetraazacyclodocecane, 1-(glutaric acid)-4,7,10-triacetic acid,
DOTAM (also called TCMC) stands for 1,4,7,10-tetrakis[carbamoylmethyl]-1,4,7,10-tetracyclodecane,
DOTAM-mono-acid stands for 1,4,7,10-tetraazacyclododecane-1,4,7-tri(carbamoylmethyl)-10-acetic acid,
DOTA-NCS stands for [4,7,10-tris-carboxymethyl-6-(4-isothiocyanato-benzyl)-1,4,7,10tetraaza-cyclododec-1-yl]-acetic acid,
DOTA-NHS stands for [4,10-bis-carboxymethyl-7-(2,5-dioxo-pyrrolidin-1-yloxycarbonylmethyl)-1,4,7,10tetraaza-cyclododec-1-yl]-acetic acid,
DOTA-NHS-ester stands for 2.2′,2″-(10-(2-((2,5-dioxopyrrolidin-1-yl) oxy)-2-oxoethyl)-1,4,7,10-tetraazacyclododecane-1,4,7-triyl) triacetic acid,
DOTMA stands for 1,4,7,10-tetraazacyclododecane-1,4,7,10-tetra(methylene phosphonic acid),
DOTP stands for 1,4,7,10-tetraazacyclododecane-1,4,7,10-tetra(methylene phosphonic acid),
DOXP stands for (di-2-pyridylketone-4,4-dimethyl-3-thiosemicarbazone,
Dp44mT stands for 2-(di-2-pyridinylmethylene)-N,N-dimethyl-hydrazinecarbothioamide, di-2-pyridylketone-4,4,-dimethyl-3-thiosemicarbazone,
DpC stands for di-2-pyridylketone-4-cyclohexyl-4-methyl-3-thiosemicarbazone,
DTC stands for diethyldithiocarbamate,
DTPA stands for diethylenetriaminepentaacetic acid,
EDDA stands for ethylenediaminediacetic acid,
FSC (also called fusarine C) stands for 3,15,27-triamino-7,19,31-trihydroxy-10,22,34-trimethyl-1,13,25-trioxa-7,19,31-triaza-cyclohexatriaconta-9,21,33-triene-2,8,14,20,26,32-hexaone,
H2ATSM stands for 1,4,8,11-tetraazacyclotetradecane-1-(methanephosphonic acid)-8-(methanecarboxylic acid),
H2CB-TE2A stands for 4,11-bis(carboxymethyl)-1,4,8,11-tetraazabicyclo[6.6.2]hexadecane,
H2CHX-DEDPA stands for N,N′-(6-carboxy-2-pyridylmethyl)-N,N′-diacetic acid-1,2-diaminoethane,
H2dedpa stands for 1,2-[{6-(carboxylato)pyridin-2-yl}methylamino]ethane,
H2DO2A stands for 1,4,7,10-tetraazacyclododecane-1,7-diacetic acid,
H2ODO2A stands for 1-oxa-4,7,10-triazacyclododecane-4,10-diacetic acid,
H2PTSM stands for pyruvaldehyde bis(methy-lthiosemicarbazone),
H4octapa stands for N,N′-(6-carboxy-2-pyridylmethyl)-N,N′-diacetic acid-1,2-diaminoethane,
HBED stands for bis(2-hydroxybenzyl) ethylenediaminediacetic acid,
HBED-CC stands for N,N′-bis[2-hydroxy-5-(carboxyethyl)benzyl]ethylenediamine-N,N′-diacetic acid,
HEHA stands for 1,4,7,10,13,16-hexaazacyclooctadecane-N,N′,N″,N′″,N″″,N′″″-hexaacetic acid,
HOPO stands for the octadentate hydroxypyridinone-type group of chelators,
HP-DO3A stands for 2,2′,2″-[10-(2-hydroxypropyl)-1,4,7,10-tetraazacyclododecane-1,4,7-triyl]triacetic acid,
HYNIC stands for 6-hydrazino-nicotinic acid,
HYNIC-Ko-DPPB stands for N-ε-(2-(diphenylphosphino)benzoyl)-N-α-(6-(2-(2-Sulfonatobenzaldehyde)hydrazono)nicotinyl)lysine methyl ester,
HYNIC-Kp-DPPB stands for N-ε-(4-(diphenylphosphino)benzoyl)-N-α-(6-(2-(2-sulfonatobenzaldehyde)hydrazono)nicotinyl)lysine methyl ester,
macropa stands for N,N′-bis[(6-carboxy-2-pyridyl)methyl]-4,13-diaza-18-crown,
MAG3 stands for (N-hydroxysuccinimidyl S-acetylmercaptoacetyltriglycinate,
maleimide-DOTA stands for 2,2′,2″-(10-(1-carboxy-4-((2-(2,5-dioxo-2,5-dihydro-1H-pyrrol-1-yl)ethyl)amino)-4-oxobutyl)-1,4,7,10-tetraazacyclododecane-1,4,7-triyl)triacetic acid,
maleimide-DOTA-GA stands for 2,2′,2″-(10-(1-carboxy-4-((2-(2,5-dioxo-2,5-dihydro-1H-pyrrol-1-yl)ethyl)amino)-4-oxobutyl)-1,4,7,10-tetraazacyclododecane-1,4,7-triyl)triacetic acid,
maleimido-mono-amide-NOTA stands for 1,4,7-triazacyclononane-1,4-bis-acetic acid-7-maleimidoethylacetamide,
maleimido-mono-amine-DOTA stands for 1,4,7,10-tetraazacyclododecane-1,4,7-tris-acetic acid-10-maleimidoethylacetamide,
MAMA stands for monoamine-monoamide dithiol,
MAMA-DA stands for N-[[[(2-mercaptoethyl)amino]carbonyl]methyl]-N-(2-mercaptoethyl)-6-aminododecanoic acid,
MAMA-HA stands for N-[[[(2-mercaptoethyl)amino]carbonyl]methyl]-N-(2-mercaptoethyl)-6-aminohexanoic acid,
MAMA-HAD stands for N-[[[(2-mercaptoethyl)amino]carbonyl]methyl]-N-(2-mercaptoethyl)-6-aminohexadecanoic acid,
MA-NOTMP stands for methylaminotriazacyclononane trimethylphosphinate,
MM-TE2A stands for 1,8-N,N′-bis-(carboxymethyl)-4-N″-(methyl)-1,4,8,11-tetraazacyclotetradecane,
N2S2 stands for N,N′-bis-(2-amino-ethyl)-propane-1,3-diamine,
N3OA stands for 4,7,10-tris(carbamoylmethyl)-,4,7,10-triaza-12-crown-ether,
N3S stands for (sulfanylidenehydrazinylidene)azanide,
N4 stands for N,N′-bis-(2-amino-ethyl)-propane-1,3-diamine,
NB-DOTA stands for {4-[2-(bis-carboxymethyl-amino)-ethyl]-7-carboxymethyl-[1,4,7]triazonan-1-yl}-acetic acid,
N-benzyl-NODA stands for 1-benzyl-1,4,7-triazonane-1,4-diyl)diacetic acid,
NE3TA stands for {4-carboxymethyl-7-[2-(carboxymethyl-amino)-ethyl]-[1,4,7]triazonan-1-yl}-acetic acid,
NETA stands for {4-[2-(bis-carboxymethyl-amino)-ethyl]-7-carboxymethyl-[1,4,7]triazonan-1-yl}-acetic acid,
NH2-DOTA-GA stands for 2,2′,2″-(10-(4-((2-aminoethyl)amino)-1-carboxy-4-oxobutyl)-1,4,7,10-tetraazacyclododecane-1,4,7-triyl)triacetic acid,
NH2-PEG4-DOTA-GA stands for 2,2′,2″-(10-(1-amino-19-carboxy-16-oxo-3,6,9,12-tetraoxa-15-azanonadecan-19-yl)-1,4,7,10-tetraazacyclododecane-1,4,7-triyl)triacetic acid,
NHS-BAT ester stands for N-hydroxysuccinimide ester of 6-(4′-(4″-carboxyphenoxy)butyl)-2, 10-dimercapto-2, 10-dimethyl-4,8-diazaundecane,
NHS-HYNIC stands for N-hydroxysuccinimidyl hydrazino nicotinate hydrochloride,
nitro-DOTA stands for 2-(g-nitrobenzyl)-1,4,7,10-tetraazacyclododecane-N,N′,N″,N′″-tetraacetic acid,
nitro-PA-DOTA stands for a-(2-(g-nitrophenyl)ethyl)-1,4,7,10-tetraazacyclododecane-1-acetic-4,7,10-tris(methylacetic) acid,
NO2A stands for 1,4,7-triazacyclononane-N,N′,N″-triacetic acid,
NO2A-azide stands for 1,4,7-triazacyclononane-1,4-bis(acetic acid)-7-(3-azidopropylacetamide),
NO2A-butyne stands for 1,4,7-triazacyclononane-1,4-bis(acetic acid)-7-(3-butynylacetamide),
NO2AP stands for triazacyclononane,
NO3AP stands for 1,4,7-triazacyclononane-N-glutaric acid-N′,N″-diacetic acid,
NODA stands for 4,10-bis(carbamoylmethyl)-4,10-diaza-12-crown-ether,
NODAGA stands for 1,4,7-triazacyclononane-N-glutaric acid-N′,N″-diacetic acid,
NODA-MPAA stands for 1,4,7-triazacyclononane-1,4-diacetate-methyl phenylacetic acid,
NOPO stands for 3-{[4,7-Bis-(hydroxy-hydroxymethyl-phosphinoylmethyl)-[1,4,7]triazonan-1-ylmethyl]-hydroxy-phosphinoyl}-propionic acid
NOTA stands for 1,4,7-triazacyclononanetriacetic acid,
NOTAM stands for 2,2′,2″-(1,4,7-triazacyclononane-1,4,7-triyl)triacetamide,
NOTA-NHS stands for 3-hydroxy-2-oxopyridine,
NOTA-NHS ester stands for 2-(4,7,10-tris(2-(tert-butoxy)-2-oxoethyl)-1,4,7,10-tetraazacyclododecan-1-yl)acetic acid,
NOTP stands for 1,4,7-triazacyclononane-N,N′N″-tris(methylene phosphonic) acid),
NTP stands for {4-carboxymethyl-7-[2-(carboxymethyl-amino)-ethyl]-[1,4,7]triazonan-1-yl}-acetic acid,
NxS4-x (N4, N2S2, N₃S) stands for a group of tetradentate chelators with N-atoms (basic amine or non-basic amide) and thiols as donors stabilizing Tc-complexes, especially Tc(V)-oxo complexes,
o,p-EDDHA stands for ethylenediamine-N(o-hydroxyphenylacetic)-N′(p-hydroxyphenylacetic) acid,
OPTT stands for 9-oxa-3,6,12,15,21-pentaazatricyclo[15,3,2,1]trieicos-1(21),17,19-triene-2,7,11,16-tetradione,
OTTA stands for 1-oxa-4,7,10-triazacyclododecane-N,N′,N″-triacetic acid,
oxo-DO3A stands for 1-oxa-4,7,10-triazacyclododecane-4,7,10-triacetic acid,
PA-DOTA stands for 1,4,7,10-tetraaza-N-(1-carboxy-3-(4-nitrophenyl)propyl)-N′,N″,N′″-tris(acetic acid)cyclododecane,
p-BZ-HTCPP stands for 3,6,9,15-tetraazabicyclo[9.3.1]-pentadeca-1(15),11,13-triene-3,6,9,-triacetic acid,
PCBA stands for 1-[(1,4,7,10,13-pentaazacyclopentadec-1-yl)methyl]benzoic acid,
PCB-TE1A1P stands for 2-(11-(phosphonomethyl)-1,4,8,11-tetraazabicyclo[6.6.3]heptadecan-4-yl)acetic acid,
PCTA stands for 3,6,9,15-tetraazabicyclo[9.3.1]-pentadeca-1(15),11,13-triene-3,6,9,-triacetic acid,
PEPA stands for 1,4,7,10,13-pentaazacyclopentadecane-N, N′, N″, N′″, N″″-pentaacetic acid,
PIH stands for pyridoxal isonicotinoyl hydrazone,
p-NCS-Bz-DFO stands for N₁-hydroxy-N1-(5-(4-(hydroxy(5-(3-(4-thiocyanatobenzoyl)thioureido)pentyl)amino)-4-oxobutanamido)pentyl)-N4-(5-(N-hydroxyacetamido)pentyl)succinamide,
p-NCS-Bz-DOTA stands for S-2-(4-thiocyanatobenzoyl)-1,4,7,10-tetraazacyclododecane tetraacetic acid,
p-NH₂-Bn-DOTA stands for S-2-(4-aminobenzyl)-1,4,7,10-tetraazacyclododecane tetraacetic acid,
p-NH2-Bn-NOTA stands for 2-S-(4-aminobenzyl)-1,4,7-triazacyclononane-1,4,7-triacetic acid,
p-NFH-Bn-oxo-DO3A stands for 1-oxa-4,7,10-tetraazacyclododecane-5-S-(4-aminobenzyl)-4,7,10-triacetic acid,
p-NH₂-Bn-PCTA stands for 3,6,9,15-tetraazabicyclo[9.3.1]pentadeca-1(15),11,13-triene-4-S-(4-aminobenzyl)-3,6,9-triacetic acid,
p-NO₂-Bn-cyclen stands for S-2-(4-nitrobenzyl)-1,4,7,10-tetraazacyclododecane,
p-NO₂-Bn-DOTA stands for S-2-(4-nitrobenzyl)-1,4,7,10-tetraazacyclododecane tetraacetic acid,
p-SCN-Bn-DFO stands for N1-hydroxy-N1-(5-(4-(hydroxy(5-(3-(4-isothiocyanatophenyl)thioureido)pentyl)amino)-4-oxobutanamido)pentyl)-N4-(5-(N-hydroxyacetamido)pentyl)succinamide,
p-SCN-Bn-DOTA stands for S-2-(4-isothiocyanatobenzyl)-1,4,7,10-tetraazacyclododecane tetraacetic acid,
p-SCN-Bn-NOTA stands for 2-S-(4-isothiocyanatobenzyl)-1,4,7-triazacyclononane-1,4,7-triacetic acid,
p-SCN-Bn-oxo-DO3A stands for 1-oxa-4,7,10-tetraazacyclododecane-5-S-(4-isothiocyanatoobenzyl)-4,7,10-triacetic acid,
p-SCN-Bn-PCTA stands for 3,6,9,15-tetraazabicyclo[9.3.1]pentadeca-1(15),11,13-triene-4-S-(4-isothiocyanatobenzyl)-3,6,9-triacetic acid,
p-SCN-BN-TCMC stands for S-2-(4-isothiocyanatobenzyl)-1,4,7,10-tetraaza-1,4,7,10-tetra(2-carbamoylmethyl)cyclododecane,
p-SCN-Bz-DOTA stands for S-2-(4-isothiocyanatobenzoyl)-1,4,7,10-tetraazacyclododecane tetraacetic acid,
p-SCN-Bz-NOTA stands for 2-S-(4-isothiocyanatobenzoyl)-1,4,7-triazacyclononane-1,4,7-triacetic acid,
p-SCN-PhPr-NE3TA stands for 2,2′-(7-(2-((carboxymethyl)(3-(4-isothiocyanatophenyl)propyl)amino)ethyl)-1,4,7-triazonane-1,4-diyl)diacetic acid,
PTSM stands for pyruvaldehyde bis(N(4)-methylthiosemicarbazone),
pycup stands for 1,8-(2,6-pyridinedimethylene)-1,4,8,11-tetraazacyclo-tetradecane, pycup2Bn stands for N1-hydroxy-N1-(5-(4-(hydroxy(5-(3-(4-isothiocyanatophenyl)thioureido)pentyl)amino)-4-oxobutanamido)pentyl)-N4-(5-(N-hydroxyacetamido)pentyl)succinamide,
Sar (also called Sarcophagine) stands for 3,6,10,13,16,19-hexaazabicyclo[6.6.6]icosane,
SarAr stands for (1-N-(4-aminobenzyl)-3, 6,10,13,16,19-hexaazabicyclo[6.6.6]eicosane-1,8-diamine),
SCN-CHX-A-DTPA-P stands for [(R)-2-amino-3-(4-isothiocyanatophenyl)propyl]-trans-(S,S)-cyclohexane-1,2-diamine-pentaacetic acid,
SCN-TETA stands for 6-[p-(isothiocyanato)benzyl]-1,4,8, 11-tetraazacyclotetradecane-1,4,8,11-tetraacetic acid, SHBED stands for 3,6,10,13,16,19-hexaazabicyclo(6,6,6)icosane,
Tachpyr stands for (N,N′N″-tris(2-pyridylmethyl)-cis,cis-1,3,5-triaminocyclohexane),
tachpyr-(5-Me) stands for 1,3,5-cis,cis,-triaminocyclobexane-N,N,N-tri-(5-methyl-2-methylpyridineimine),
TACN stands for 1,4,7-triazacyclononane,
TACN-TM stands for 1,4,7-tris (2-mercaptoethyl)-1,4,7-triazacyclononane,
TA-DOTA stands for 2,2′,2″-(10-(2-((2-(5-(1,2-dithiolan-3-yl)pentanamido)ethyl)amino)-2-oxoethyl)-1,4,7,10-tetraazacyclododecane-1,4,7-triyl)triacetic acid,
TA-DOTA-GA stands for 1,1,1-tris(aminomethyl)ethane,
TAM A stands for methyl-(2-methyl-3-methylamino-2-methylaminomethyl-propyl)-amine,
TAME stands for 1,1,1-tris-(aminomethyl)ethane,
TAME-Hex stands for (1,8-N,N′-bis(carboxymethyl)-1,4,8,11-tetraazacyclotetradecane,
TBPD stands for 3,6,9,15-tetraazabicyclo[9.3.1]pentadeca-1(15),11,13-triene-2,10-dione,
TCMC stands for 1,4,8,11-tetraazacyclotetradecane-1,4,8,11-tetraacetic acid,
TE2A stands for [4,8-bis-carboxymethyl-11-(2,5-dioxo-3-sulfo-pyrrolidin-1-yloxycarbonylmethyl)-1,4,8,11tetraaza-cyclotetradec-1-yl]-acetic acid,
TEAMA stands for 6-(p-bromoacetamidobenzyl)-1,4,8,11-tetraazacyclotetradecane-1,4,8,11-tetraacetic acid,
TETA stands for 1,4,8,11-tetraazacyclododecane-1,4,8,11-tetraacetic acid,
TETAM stands for 2,2′,2″,2′″-(1,4,8,11-tetraazacyclotetradecane-1,4,8,11-tetrayl)tetraacetamide,
THP stands for hexadentate tris(3,4-hydroxypyridinone),
THPN stands for 1,3-propanediamine-N,N,N′,N′-tetrakis[(2-(aminomethyl)-3-hydroxy-1,6-dimethyl-4(1H)-pyridinone)acetamide],
THP-TATE stands for 3,3′,3″-(((1,4,7-triazonane-1,4,7-triyl)tris(methylene))tris(hydroxyphosphoryl))tripropanoic acid,
TRAP stands for 3-({4,7-bis-[(2-carboxy-ethyl)-hydroxy-phosphinoylmethyl]-[1,4,7]triazonan-1-ylmethyl}-hydroxy-phosphinoyl)-propionic acid,
Triapine stands for dipyridyl thiosemicarbazone,
Tricine stands for picolylaminediacetic acid,
TRITA stands for 2,2′,2″,2′″-(1,4,7,10-tetraazacyclotridecane-1,4,7,10-tetrayl)tetraacetic acid,
TRITRAM stands for 2,2′,2″-(1,4,7,10-tetraazacyclotridecane-1,4,7-triyl)triacetamide. HYNIC, DTPA, EDTA, DOTA, TETA, bisamino bisthiol (BAT)-based chelators as disclosed in U.S. Pat. No. 5,720,934; desferrioxamine (DFO) as disclosed in Doulias et al. (Doulias, et al., Free Radic BiolMed, 2003, 35: 719), tetrapyridine and N₃S, N₂S2 and N₄ chelators as disclosed in U.S. Pat. Nos. 5,367,080 A, 5,364,613 A, 5,021,556 A, 5,075,099 A, 5,886,142 A, whereby all of the references are included herein by reference in their entirety. 6-amino-6-methylperhydro-1,4-diazepine-N,N′,N″,N″-tetraacetic acid (AAZTA) is disclosed in Pfister et al. (Pfister, et al., EJNMMI Res, 2015, 5: 74), deferiprone, a 1,2-dimethyl-3,4-hydroxypyridinone and hexadentate tris(3,4-hydroxypyridinone) (THP) are disclosed in Cusnir et al. (Cusnir, et al., Int J Mol Sci, 2017, 18), monoamine-monoamide dithiol (MAMA)-based chelators are disclosed in Demoin et al. (Demoin, et al., Nucl Med Biol, 2016, 43: 802), macropa and analogues are disclosed in Thiele et al. (Thiele, et al., Angew Chem Int Ed Engl, 2017, 56: 14712), 1,4,7,10,13,16-hexaazacyclohexadecane-N,N′,N″,N′″,N″″,N′″″-hexaacetic acid (HEHA) and PEPA analogues are disclosed in Price and Orvig (Price, et al., Chem Soc Rev, 2014, 43: 260), pycup and analogous are disclosed in Boros et al. (Boros, et al., Mol Pharm, 2014, 11: 617), N, N-bis(2-hydroxybenzyl)ethylenediamine-N,N-diacetic acid (HBED), 1,4,7,10-tetrakis (carbamoylmethyl)-1,4,7,10-tetraazacyclododecane (TCM), 2-[(carboxymethyl)]-[5-(4-nitrophenyl-1-[4,7,10-tris-(carboxymethyl)-1,4,7,10-tetraazacyclododecan-1-yl]pentan-2-yl)-amino]acetic acid (3p-C-DEPA), CB-TE2A, TE2A, TElA1P, DiAmSar, 1-N-(4-aminobenzyl)-3,6,10,13,16,19-hexaazabicyclo[6.6.6]-eicosane-1,8-diamine (SarAr), NETA, tris(2-mercaptoethyl)-1,4,7-triazacyclononane (TACN-TM), {4-[2-(bis-carboxymethyl-amino)-ethyl]-7-carboxymethyl-[1,4,7]triazonan-1-yl}-acetic acid (NETA), diethylenetriaminepentaacetic acid (DTP), 3-({4,7-bis-[(2-carboxy-ethyl)-hydroxy-phosphinoylmethyl]-[1,4,7]triazonan-1-ylmethyl}-hydroxy-phosphinoyl)-propionic acid (TRAP), NOPO, H4octapa, SHBED, BPCA, 3,6,9,15-tetraazabicyclo[9.3.1]-pentadeca-1(15),11,13-triene-3,6,9,-triacetic acid (PCTA), and 1,4,7,10,13-pentaazacyclopentadecane-N,N′,N″,N′″,N″″-pentaacetic acid (PEPA) are disclosed in Price and Orvig (Price, et al., Chem Soc Rev, 2014, 43: 260), 1-hydroxy-2-pyridone ligand (HOPO) is disclosed in Allott et al. (Allott, et al., Chem Commun (Camb), 2017, 53: 8529), [4-carboxymethyl-6-(carboxymethyl-methyl-amino)-6-methyl-[1,4]diazepan-1-yl]-acetic acid (DATA) is disclosed in Tornesello et al. (Tornesello, et al., Molecules, 2017, 22: 1282), tetrakis(aminomethyl)methane (TAM) and analogues are disclosed in McAuley 1988 (McAuley, et al., Canadian Journal of Chemistry, 1989, 67: 1657), hexadentate tris(3,4-hydroxypyridinone) (THP) and analogues are disclosed in Ma et al. (Ma, et al., Dalton Trans, 2015, 44: 4884).

The diagnostic and/or therapeutic use of some of the above chelators is described in the prior art. For example, 2-hydrazino nicotinamide (HYNIC) has been widely used in the presence of a coligand for incorporation of ^{99m}Tc and ^{186,188}Re (Schwartz, et al., Bioconjug Chem, 1991, 2: 333; Babich, et al., J Nucl Med, 1993, 34: 1964; Babich, et al., Nucl Med Biol, 1995, 22: 25); DTPA is used in Octreoscan® for complexing ¹¹¹In and several modifications are described in the literature (L1, et al., Nucl Med Biol, 2001, 28: 145; Brechbiel, et al., Bioconjug Chem, 1991, 2: 187); DOTA-type chelators for radiotherapy applications are described by Tweedle et al. (U.S. Pat. No. 4,885,363); other polyaza macrocycles for chelating trivalent isotopes metals are described by Eisenwiener et al. (Eisenwiener, et al., Bioconjug Chem, 2002, 13: 530); and N₄-chelators such as a ^{99m}Tc-N₄-chelator have been used for peptide labeling in the case of minigastrin for targeting CCK-2 receptors (Nock, et al., J Nucl Med, 2005, 46: 1727).

In an embodiment the chelator is a metal chelator selected from the group, but not limited to, comprising DOTA, DOTAGA, DOTAM, DOTP, NOTA, NODAGA, NODA-MPAA, HBED, TETA, CB-TE2A, DTPA, CHX-A″-DTPA, DFO, Macropa, HOPO, TRAP, THP, DATA, NOPO, NOTP, PCTA, sarcophagine, FSC, NETA, NE3TA, H4octapa, pycup, HYNIC, NxS4-x (N4, N2S2, N₃S), ^{99m}Tc(CO)₃-chelators and their analogs.

The chemical structures of said chelators being as follows:

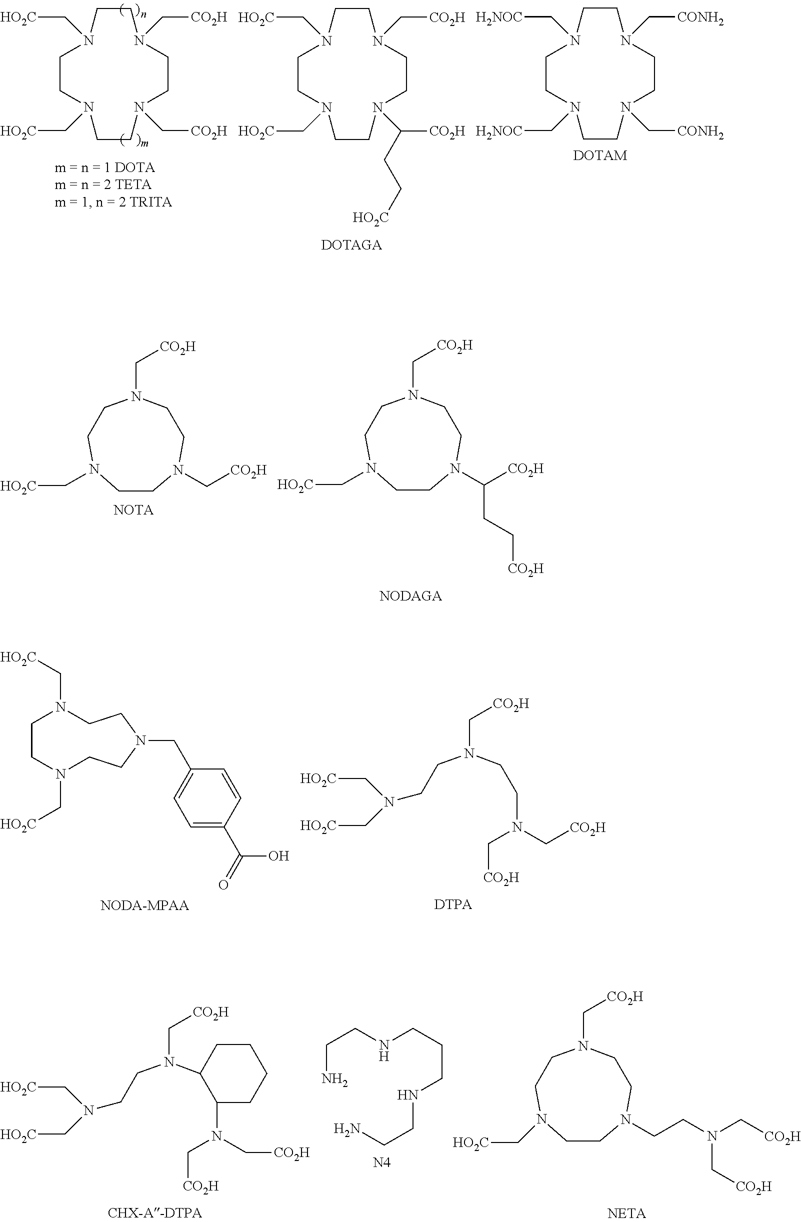

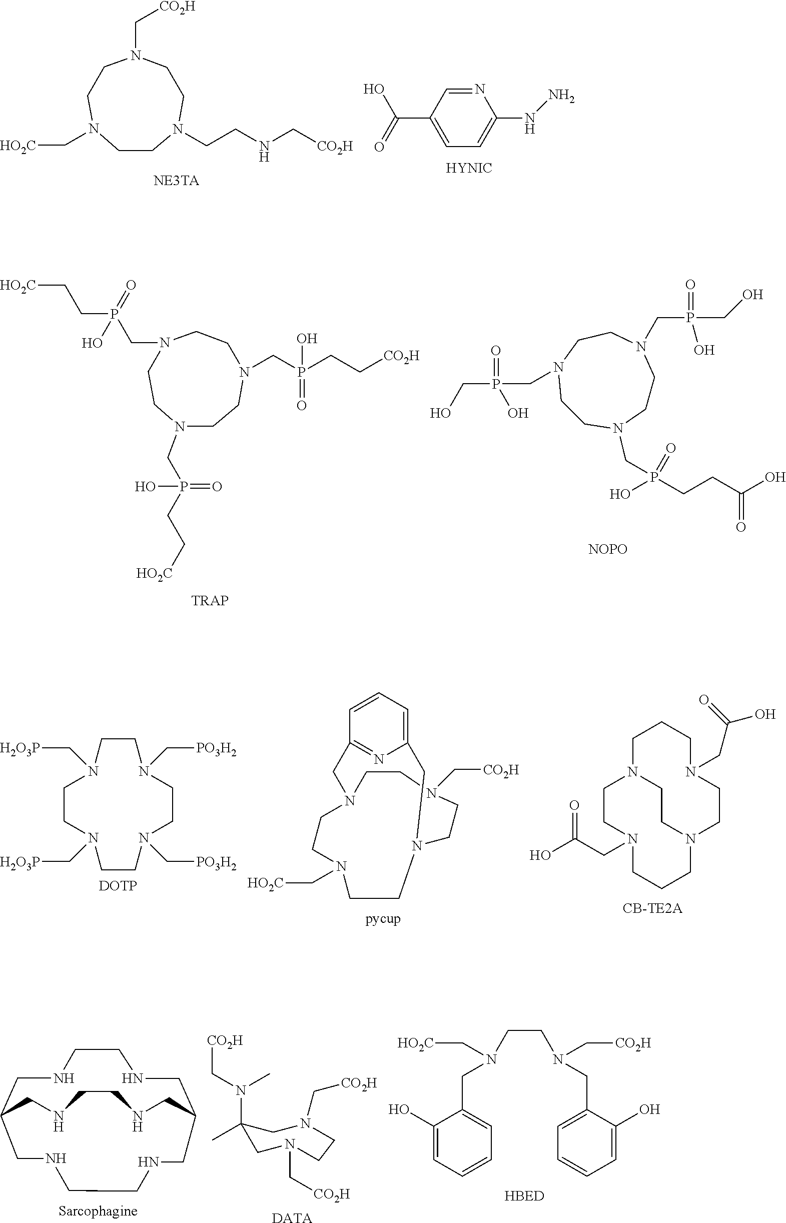

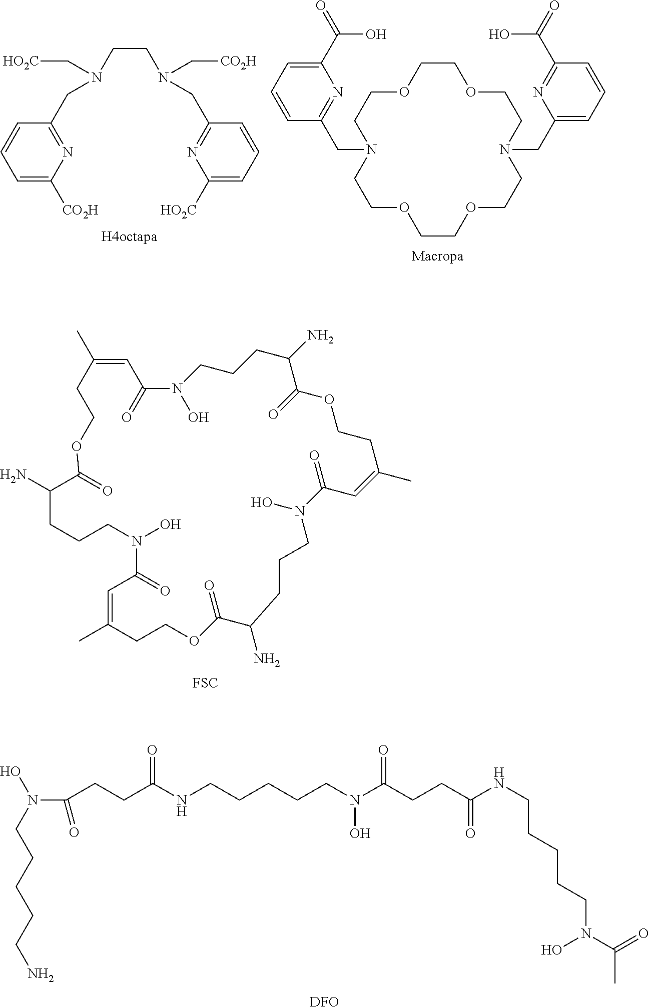

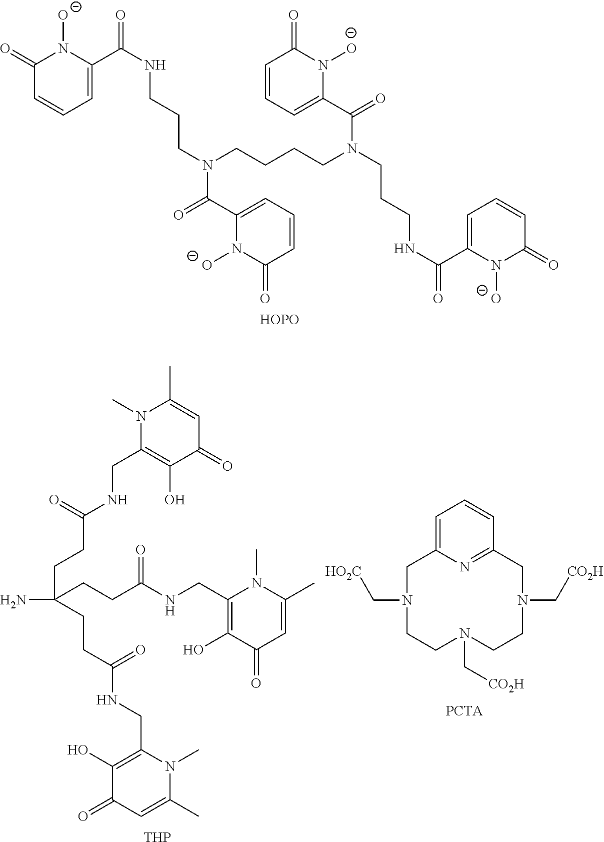

In a preferred embodiment, the metal chelator is selected from the group consisting of DOTA, DOTAGA, DOTAM, NOTA, NODAGA, NODA-MPAA, NOPO, HTBED, DTPA, CHX-A″-DTPA, CB-TE2A, Macropa, PCTA, N4, and analogs thereof.

In a more preferred embodiment, the metal chelator is selected from the group consisting of DOTA, DOTAGA, NODAGA, and macropa and their analogs thereof.

It will be acknowledged by a person skilled in the art that in an embodiment a chelator additionally comprises one or more functional groups or functionalities allowing its attachment to the compounds of the invention.

It will be acknowledged by the persons skilled in the art that the chelator, in principle, may be used regardless of whether the compound of the invention is used in or suitable for diagnosis or therapy. Such principle is, among others, outlined in international patent application WO 2009/109332 A1.

It will be further acknowledged by the persons skilled in the art that the presence of a chelator in the compound of the invention includes, if not stated otherwise, the possibility that the chelator is complexed to any metal complex partner, i.e. any metal which, in principle, can be complexed by the chelator. An explicitly mentioned chelator of a compound of the invention or the general term chelator in connection with the compound of the invention refers either to the uncomplexed chelator as such or to the chelator to which any metal complex partner is bound, wherein the metal complex partner is any radioactive or non-radioactive metal complex partner. Preferably the chelator-metal complex, i.e. the chelator to which the metal complex partner is bound, is a stable chelator-metal complex.

Non-radioactive chelator-metal complexes have several applications, e.g., for assessing properties like stability or activity which are otherwise difficult to determine. One aspect is that cold variants of the radioactive versions of the metal complex partner (e.g., non-radioactive indium complexes es described in the examples) can act as surrogates of the radioactive compounds. Furthermore, they are valuable tools for identifying metabolites in vitro or in vivo, as well as for assessing toxicity properties of the compounds of invention.

Additionally, chelator-metal complexes can be used in binding assays utilizing the fluorescence properties of some metal complexes with distinct ligands (e.g., Europium salts).

Chelators can be synthesized or are commercially available with a wide variety of (possibly already activated) groups for the conjugation to peptides or amino acids.

Direct conjugation of a chelator to an amino-nitrogen of the respective compound of invention is well possible for chelators selected from the group consisting of DTPA, DOTA, DOTAGA, NOTA, NODAGA, NODA-MPAA, HBED, TETA, CB-TE2A, DFO, DATA, sarcophagine and N4, preferably DTPA, DOTA, DOTAGA, NOTA, NODAGA, NODA-MPAA, CB-TE2A, and N₄. The preferred linkage in this respect is an amide linkage.

Direct conjugation of an isothiocyanate-functionalized chelator to an amino-nitrogen of the respective compound of invention is well possible for chelators selected from the group consisting of DOTA, DOTAGA, NOTA, NODAGA, DTPA, CHX-A″-DTPA, DFO, and THP, preferably DOTA, DOTAGA, NOTA, NODAGA, DTPA, and CHX-A″-DTPA. The preferred linkage in this respect is a thiourea linkage.

Functional groups at a chelator which are preferred precursors for the direct conjugation of a chelator to an amino-nitrogen are known to the person skilled in the art and include but are not limited to carboxylic acid, activated carboxylic acid, e.g., active ester like for instance NHS-ester, pentafluorophenol-ester, HOBt-ester, HOAt-ester, and isothiocyanate.

Functional groups at a chelator which are preferred precursors for the direct conjugation of a chelator to a carboxylic group are known to the person skilled in the art and include but are not limited to alkylamino and arylamino nitrogens. Respective chelator reagents are commercially available for some chelators, e.g., for DOTA with either alkylamino or arylamino nitrogen.

Functional groups at a chelator which are preferred precursors for the direct conjugation of a chelator to a thiol group are known to the person skilled in the art and include but are not limited to maleimide nitrogens. Respective chelator reagents are commercially available for some chelators, e.g., for DOTA with maleimide nitrogen.

Functional groups at a chelator which are preferred precursors for the direct conjugation of a chelator to an azide group are known to the person skilled in the art and include but are not limited to acyclic and cyclic alkynes. Respective chelator reagents are commercially available for some chelators, e.g., for DOTA with propargyl or butynyl.

Functional groups at a chelator which are preferred precursors for the direct conjugation of a chelator to an alkyne group are known to the person skilled in the art and include but are not limited to alkyl and aryl azines. Respective chelator reagents are commercially available for some chelators, e.g., for DOTA with azidopropyl.

In an embodiment, the compound of the invention is present as a pharmaceutically acceptable salt.

According to one embodiment, the effector is a drug, preferably a cytotoxic drug. The cytotoxic drug can be covalently bound to the cyclic peptide structure, optionally by means of linker moieties which may be cleavable or not. According to this embodiment, the compound of the present invention preferably does not comprise a chelator. In these embodiments, the drug, preferably the cytotoxic drug, may be covalently bound to the cyclic peptide structure by means of linker moieties such as L1, L3, L4, or L6 (as described above).

Hereinafter are exemplary drugs that can be used as effector in the compound of the present invention:
(A) Antineoplastic agents such as
   (A1) DNA-alkylating agents, e.g. duocarmycin (including synthetic analogues thereof: adozelesin, carzelesin, bizelesin, KW-2189 and CBI-TMI), nitrogen mustard analogues (e.g. cyclophosphamide chlorambucil, melphalan, chlormethine, ifosfamide, trofosfamide, prednimustine, bendamustine, chlornaphazine, estramustine, mechlorethamine, mechlorethamine oxide hydrochloride, mannomustine, mitolactol, novembichin, phenesterine, uracil mustard), alkyl sulphonates (e.g. busulfan, treosulfan, mannosulfan, improsulfan and piposulfan), ethylene imines (e.g. thiotepa, triaziquone, carboquone), nitrosoureas (e.g. carmustine, lomustine, semustine, streptozocin, chlorozotocin, fotemustine, nimustine, ranimustine), epoxides (e.g. etoglucid), other alkylating agents (e.g. mitobronitol, pipobroman, temozolomide, dacarbazine);
(A2) Topoisomerase inhibitors, e.g. doxorubicin, morpholino-doxorubicin, cyanomorpholino-doxorubicin, 2-pyrrolino-doxorubicin, deoxydoxorubicin, etoposide, etoposide phosphate, irinotecan and metabolites thereof such as SN-38, teniposide, topotecan, resveratrol, epipodophyllins (e.g. 9-aminocamptothecin, camptothecin, crisnatol, daunomycin, mitoxantrone, novantrone, retinoic acids (retinols), 9-nitrocamptothecin (RFS 2000));
(A3) RNA-polymerase II inhibitors, e.g. alpha-amanitin, other amatoxins;
(A4) DNA-cleaving agents, e.g. calicheamicin;
(A5) Antimitotic agents or microtubule disruptors, e.g. vinca alkaloids (e.g. vincristine, vinblastine, vindesine, vinorelbine, navelbin, vinflunide, vintafolide), taxanes (e.g. paclitaxel, docetaxel, paclitaxel polyglumex, cabazitaxel) and their analogs, maytansinoids (e.g. DM1, DM2, DM3, DM4, maytansine and ansamitocins) and their analogs, cryptophycins (e.g. cryptophycin 1 and cryptophycin 8), epothilones, eleutherobin, discodermolide, bryostatins, dolostatins, auristatins (e.g. monomethyl auristatin E (MMAE), monomethyl auristatin F), tubulysins, cephalostatins; pancratistatin, sarcodictyin, spongistatin, demecolcine, mitomycins;
(A6) Anti-metabolites, e.g. DHFR inhibitors (e.g. methotrexate, trimetrexate, denopterin, pteropterin, aminopterin (4-aminopteroic acid) or other folic acid analogues such as raltitrexed, pemetrexed, pralatrexate), IMP dehydrogenase inhibitors (e.g. mycophenolic acid, tiazofurin, ribavirin, EICAR), ribonucleotide reductase inhibitors (e.g. hydroxyurea, deferoxamine), pyrimidine analogs (e.g. cytarabine, fluorouracil, 5-fluorouracil and metabolites thereof, tegafur, carmofur, gemcitabine, capecitabine, azacitidine, decitabine, fluorouracil combinations, tegafur combinations, trifluridine combinations, cytosine arabinoside, ancitabine, floxuridine, doxifluridine), uracil analogs (e.g. 6-azauridine, deoxyuridine), cytosine analogs (e.g. enocitabine), purine analogs (e.g. azathioprine, fludarabine, mercaptopurine, thiamiprine, thioguanine, cladribine, clofarabine, nelarabine), folic acid replenisher such as folinic acid;
(A7) A kinesin spindle protein inhibitor, e.g. filanesib;
(A8) Kinase inhibitors, e.g. ipatasertib, BIBW 2992 (anti-EGFR/Erb2), imatinib, gefitinib, pegaptanib, sorafenib, dasatinib, sunitinib, erlotinib, nilotinib, lapatinib, axitinib, pazopanib, vandetanib, afatinib, vemurafenib, crizotinib, regorafenib, masitinib, dabrafenib, trametinib, ibrutinib, ceritinib, lenvatinib, nintedanib, cediranib, palbocidib, osimertinib, alectinib, alectinib, rociletinib, cobimetinib, midostaurin, olmutinib, E7080 (anti-VEGFR2), mubritinib, ponatinib (AP24534), bafetinib (INNO-406), bosutinib (SKI-606), cabozantinib, vismodegib, iniparib, ruxolitinib, CYT387, tivozanib, ispinesib, temsirolimus, everolimus, ridaforolimus;
(A9) A nicotinamide phosphoribosyltransferase inhibitor, e.g. CAS No. 2241014-82-2;
(A10) A matrix metallopeptidase 9 inhibitor, e.g. derivatives of CGS27023A;
(A11) A phosphatase inhibitor, e.g. mycrocystin-LR;
(B) Immunomodulatory agents including immunostimulants, immunosuppressants, cyclosporine, cyclosporine A, aminocaproic acid, azathioprine, bromocriptine, chlorambucil, chloroquine, cyclophosphamide, corticosteroids (e.g. amcinonide, betamethasone, budesonide, hydrocortisone, flunisolide, fluticasone propionate, fluocortolone danazol, dexamethasone, prednisone, triamcinolone acetonide, beclometasone dipropionate), DHEA, hydroxychloroquine, meloxicam, methotrexate, mofetil, mycophenylate, sirolimus, tacrolimus, everolimus, fingolimod, ibrutinib, imiquimod, resiquimod, cytokines, peptidic immunomodulators such as TLR agonists (e.g. CpG oligonucleotides);
(C) Anti-infectious disease agents including antibacterial drugs, antimycobacterial drugs and antiviral drugs. A non-limiting example of antibiotic used in an antibiotic-antibody drug conjugate is rifalogue, i.e. a rafamycin derivative;
(D) Radioisotopes, metabolites, pharmaceutically acceptable salts, and/or prodrugs of any of the aforementioned agents (A) to (C).

According to one embodiment, the effector is a moiety derived from exatecan, PNU-159682, DM4, amanitin, duocarmycin, auristatin, maytansine, tubulysin, calicheamicin, SN-38, taxol, daunomycin, vinblastine, doxorubicine, methotrexate, pyrrolobenzodiazepine, pyrrole-based kinesin spindle protein (KSP) inhibitors, indolino-benzodiazepine dimers, or radioisotopes and/or pharmaceutically acceptable salts thereof.

### 4. USE OF COMPOUND OR PEPTIDE FOR DIAGNOSTIC AND/OR THERAPEUTIC PURPOSES

In an embodiment and as preferably used herein, a diagnostically active compound is a compound which is suitable for or useful in the diagnosis of a disease.

In an embodiment and as preferably used herein, a diagnostic agent or a diagnostically active agent is a compound which is suitable for or useful in the diagnosis of a disease.

In an embodiment and as preferably used herein, a therapeutically active compound is a compound which is suitable for or useful in the treatment of a disease.

In an embodiment and as preferably used herein, a therapeutic agent or a therapeutically active agent is a compound which is suitable for or useful in the treatment of a disease.

In an embodiment and as preferably used herein, a theragnostically active compound is a compound which is suitable for or useful in both the diagnosis and therapy of a disease.

In an embodiment and as preferably used herein, a theragnostic agent or a theragnostically active agent is a compound which is suitable for or useful in both the diagnosis and therapy of a disease.

In an embodiment and as preferably used herein, theragnostics is a method for the combined diagnosis and therapy of a disease; preferably, the combined diagnostically and therapeutically active compounds used in theragnostics are radiolabeled.

In an embodiment and as preferably used herein, treatment of a disease is treatment and/or prevention of a disease.

In an embodiment and as preferably used herein, pEC50 is determined in a FACS binding assay, wherein the FACS binding assay is as described in the example part.

In an embodiment and as preferably used herein, pIC50 is determined in a FACS binding assay, wherein the FACS binding assay is as described in the example part.

In an embodiment and as preferably used herein, a disease involving CAIX is a disease where cells including but not limited to tumor cells expressing, preferably in an upregulated manner, CAIX and tissue either expressing CAIX, preferably in an upregulated manner respectively, are either a or the cause for the disease and/or the symptoms of the disease, or are part of the pathology underlying the disease. A preferred CAIX-expressing cell is a tumor cell. In an embodiment of the disease, preferably when used in connection with the treatment, treating and/or therapy of the disease, affecting the cells, the tissue and pathology, respectively, results in cure, treatment or amelioration of the disease and/or the symptoms of the disease. In an embodiment of the disease, preferably when used in connection with the diagnosis and/or diagnosing of the disease, labeling of the CAIX-expressing cells and/or of the CAIX-expressing tissue allows discriminating or distinguishing said cells and/or said tissue from healthy or CAIX-non-expressing cells and/or healthy or CAIX non-expressing tissue. More preferably such discrimination or distinction forms the basis for said diagnosis and diagnosing, respectively. In an embodiment thereof, labeling means the interaction of a detectable label either directly or indirectly with the CAIX-expressing cells and/or with the CAIX-expressing tissue or tissue containing such CAIX-expressing cells; more preferably such interaction involves or is based on the interaction of the label or a compound bearing such label with CAIX.

In an embodiment and as preferably used herein, a target cell is a cell which is expressing CAIX and is a or the cause for a disease and/or the symptoms of a disease, or is part of the pathology underlying a disease.

In an embodiment and as preferably used herein, a non-target cell is a cell which is either not expressing CAIX and/or is not a or the cause for a disease and/or the symptoms of a disease, or is part of the pathology underlying a disease.

In an embodiment and as preferably used herein, a neoplasm is an abnormal new growth of cells. The cells in a neoplasm grow more rapidly than normal cells and will continue to grow if not treated. A neoplasm may be benign or malignant.

In an embodiment and as preferably used herein, a tumor is a mass lesion that may be benign or malignant.

In an embodiment and as preferably used herein, a cancer is a malignant neoplasm.

The CAIX expression pattern in solid tumors makes it a compelling therapeutic and diagnostic target. CAIX has been reported to be upregulated in most types of solid tumors including but not limited to breast (Storci et al., J Pathol, 2008, 214, 25-37), kidney (Luong-Player et al., Am J Clin Pathol, 2014, 141, 219-225), colon (Korkeila et al., Br J Cancer, 2009, 100, 874-880), ovarian (Choschzick et al., Virchows Arch, 2011, 459, 193-200), head-and-neck (Kappler et al., Strahlenther Onkol, 2008, 184, 393-399), pancreatic (Juhasz et al., Aliment Pharmacol Ther, 2003, 18, 837-846) and lung cancer (Ilie et al., Br J Cancer, 2010, 102, 1627-1635).

One of the best-studied indications for CAIX in the field of kidney malignancies are renal cell carcinomas (RCC). CAIX expression in clear cell RCC is in contrast to other neoplasms uncoupled from the hypoxia-induced signaling cascade (Shuin et al., Cancer Res, 1994, 54, 2852-2855). In a study, 317 primary renal tumors were investigated for their CAIX expression levels via immunohistochemistry (IHC). High expression of CAIX (>85% of tumor cells) was found in 71% of RCC samples (Genega et al., Am J Clin Pathol, 2010, 134, 873-879). This finding and the fact that clear cell renal cell carcinomas account for the majority of epithelial neoplasms of the kidney make clear cell RCC an appealing indication for a targeted CAIX compound.

Furthermore, a study investigated CAIX expression in 166 rectal cancer patients treated by preoperative radio- or chemo-radiotherapy or surgery only (Korkeila et al., British Journal of Cancer, 2009, 100, 874-880). It was found that 44% of the surgical patient's tumor samples (39 out of 80) were CAIX positive.

In a broad immunohistochemically study of 1551 cases of tumor and normal samples from various organs the expression of carbonic anhydrase 9 expression was evaluated (Luong-Player et al., Am J Clin Pathol, 2014, 141, 219-225). The indications with the highest amount of CAIX positive staining was intrahepatic cholangiocarcinoma (90%) and the above discussed clear cell renal cell carcinoma (90% low grade and 86% high-grade tumors). In the range of 30-70% positive CAIX tumor samples following indications were found: endocervical adenocarcinoma (68%), pancreatic adenocarcinoma (58%), squamous cell carcinoma (57%), gastric adenocarcinoma (57%), endometrial carcinoma FIGO II (54%), colonic adenocarcinoma (51%), ovary papillary serous carcinoma (49%) endometrial carcinoma FIGO I (47%), lung adenocarcinoma mixed type (46%) esophageal adenocarcinoma (43%), infiltrating urothelial carcinoma (35%) and papillary renal cell carcinoma (30%).

Next to the expression on cancer cells, CAIX upregulation on cancer-associated fibroblasts (CAFs) was reported. CAF cells are one of the most prominent components of the tumor microenvironment (TME). This TME is a pivotal factor for the tumor's capability to continuously grow. Targeting of CAFs is a widely accepted strategy to inhibit tumor growth. CAIX expression in the tumor microenvironment opens up yet another option to target malignant tissues. The upregulation of CAIX in both pancreatic tumor cells and their surrounding cancer-associated fibroblasts has been reported (Fiaschi et al., Cell Cycle, 2013, 12, 1791-1801). Furthermore, in a study of lung adenocarcinoma CAIX positive CAF staining with immunohistochemistry was shown for 39 out of 158 tissue samples (Nakao et al., Cancer, 2009, 115, 2732-2743). Additionally, the expression of CAIX correlated with a significantly poorer outcome for patients.

The compounds of the invention have a high binding affinity to CAIX. Because of this high binding affinity, the compounds of the invention are effective as, useful as and/or suitable as a targeting agent and, if conjugated to another moiety, as a targeting moiety. As preferably used herein a targeting agent is an agent which interacts with the target molecule which is in the instant case said CAIX. In terms of cells and tissues thus targeted and targetable, respectively, by the compounds of the invention any cell and tissue, respectively, expressing said CAIX in particular is targeted and targetable, respectively. As is known from the prior art, apart from specific tissues of the gastrointestinal tract, and, to a lower extent, the CNS (Zamanova et al., Expert Opin Ther Pat, 2019, 29, 509-533), CAIX is highly expressed in a mammalian body and a human body in particular on several neoplastic cells in several tumor indications, whereas the expression of CAIX in other tissues of the mammalian and the human body is low. These CAIX-expressing tumor indications include but are not limited to breast (Storci et al., J Pathol, 2008, 214, 25-37), kidney (Luong-Player et al., Am J Clin Pathol, 2014, 141, 219-225), colon (Korkeila et al., Br J Cancer, 2009, 100, 874-880), ovarian (Choschzick et al., Virchows Arch, 2011, 459, 193-200), head-and-neck (Kappler et al., Strahlenther Onkol, 2008, 184, 393-399), pancreatic (Juhasz et al., Aliment Pharmacol Ther, 2003, 18, 837-846) and lung cancer (Ilie et al., Br J Cancer, 2010, 102, 1627-1635). In clear cell renal cell carcinomas, CAIX expression is unique compared to other cancers as it is commonly uncoupled from the hypoxia-induced signaling cascade (Shuin et al., Cancer Res, 1994, 54, 2852-2855).

Accordingly, the compounds of the invention are thus particularly suitable for and useful in the diagnosis and treatment, respectively, of these diseases. Insofar, the above indications are indications which can be treated by the compound of the invention. It will be understood by the person skilled in the art that also metastases and metastases of the above indications in particular can be treated and diagnosed by the compound of the invention and the methods of diagnosis and methods of treatment making use of the compound of the invention.

It is also within the present invention that the compound of the invention is used or is for use in a method for the treatment of a disease as disclosed herein. Such method, preferably, comprises the step of administering to a subject in need thereof a therapeutically effective amount of the compound of the invention. Such method includes, but is not limited to, curative or adjuvant cancer treatment. It is used as palliative treatment where cure is not possible and the aim is for local disease control or symptomatic relief or as therapeutic treatment where the therapy has survival benefit and it can be curative.

The method for the treatment of a disease as disclosed herein includes the treatment of the disease disclosed herein, including tumors and cancer, and may be used either as the primary therapy or as second, third, fourth or last line therapy. It is also within the present invention to combine the compound of the invention with further therapeutic approaches. It is well known to the person skilled in the art that the precise treatment intent including curative, adjuvant, neoadjuvant, therapeutic, or palliative treatment intent will depend on the tumor type, location, and stage, as well as the general health of the patient.

Without wishing to be bound by any theory, the therapeutic effect of the compounds of present invention is based on the delivery of a radionuclide to a diseased CAIX expressing cell or structure which is destroyed by the radiation emitted by the radionuclide.

Without wishing to be bound by any theory, the therapeutic use of the compounds of the invention arises from the binding of said compounds to CAIX expressing cells, cancer cells in particular, wherein said cells are killed by the radiation emitted by the radionuclide. It will also be appreciated by a person skilled in the art that CAIX is a pan-tumor target which is expressed under hypoxic conditions, whereby such hypoxic are a hallmark of cancer. Because of this, any cancer and tumor can be treated and diagnosed, respectively, preferably any hypoxic cancer and tumor. In a further embodiment, the disease is a solid cancer, preferably a hypoxic solid cancer.

Furthermore, the therapeutic use of the compounds of the invention arises from the binding of said compounds to CAIX expressing cancer-associated fibroblasts (CAFs). It will also be appreciated by a person skilled in the art that CAFs are a cell type which is present within the tumor microenvironment promoting tumorigenic features by initiating the remodelling of the extracellular matrix or by secreting cytokines. Because of this, any tumor can be treated and diagnosed, respectively, preferably any cancer and tumor, respectively, comprising CAIX-expressing CAFs. In light thereof, in a further embodiment, the disease which may be diagnosed and treated, respectively, by the compounds of the invention is a cancer comprising CAIX-expressing CAFs. Again, without wishing to be bound by any theory, the therapeutic use of the compounds of the invention arises from the binding of said compounds to CAIX-expressing CAFs, wherein the CAFs are killed by the radiation emitted by the radionuclide born by the chelator of the compound of the invention.

In an embodiment of the present invention, the disease is selected from the group comprising neoplasm nos, neoplasm, benign, neoplasm, uncertain whether benign or malignant, neoplasm, malignant, neoplasm, metastatic, neoplasm, malignant, uncertain whether primary or metastatic, tumor cells, benign, tumor cells, uncertain whether benign or malignant, tumor cells, malignant, malignant tumor, small cell type, malignant tumor, giant cell type, malignant tumor, fusiform cell type, epithelial neoplasms nos, epithelial tumor, benign, carcinoma in situ nos, carcinoma nos, carcinoma, metastatic nos, carcinomatosis, epithelioma, benign, epithelioma, malignant, large cell carcinoma nos, carcinoma, undifferentiated type nos, carcinoma, anaplastic type nos, pleomorphic carcinoma, giant cell and spindle cell carcinoma, giant cell carcinoma, spindle cell carcinoma, pseudosarcomatous carcinoma, polygonal cell carcinoma, spheroidal cell carcinoma, tumorlet, small cell carcinoma nos, oat cell carcinoma, small cell carcinoma, fusiform cell type, papillary and squamous cell neoplasms, papilloma nos, papillary carcinoma in situ, papillary carcinoma nos, verrucous papilloma, verrucous carcinoma nos, squamous cell papilloma, papillary squamous cell carcinoma, inverted papilloma, papillomatosis nos, squamous cell carcinoma in situ nos, squamous cell carcinoma nos, squamous cell carcinoma, metastatic nos, squamous cell carcinoma, keratinizing type nos, squamous cell carcinoma, large cell, nonkeratinizing type, squamous cell carcinoma, small cell, nonkeratinizing type, squamous cell carcinoma, spindle cell type, adenoid squamous cell carcinoma, squamous cell carcinoma in situ with questionable stromal invasion, squamous cell carcinoma, microinvasive, queyrat's erythroplasia, bowen's disease, lymphoepithelial carcinoma, basal cell neoplasms, basal cell tumor, basal cell carcinoma nos, multicentric basal cell carcinoma, basal cell carcinoma, morphea type, basal cell carcinoma, fibroepithelial type, basosquamous carcinoma, metatypical carcinoma, intraepidermal epithelioma of jadassohn, trichoepithelioma, trichofolliculoma, tricholemmoma, pilomatrixoma, transitional cell papillomas and carcinomas, transitional cell papilloma nos, urothelial papilloma, transitional cell carcinoma in situ, transitional cell carcinoma nos, schneiderian papilloma, transitional cell papilloma, inverted type, schneiderian carcinoma, transitional cell carcinoma, spindle cell type, basaloid carcinoma, cloacogenic carcinoma, papillary transitional cell carcinoma, adenomas and adenocarcinomas, adenoma nos, bronchial adenoma nos, adenocarcinoma in situ, adenocarcinoma nos, adenocarcinoma, metastatic nos, scirrhous adenocarcinoma, linitis plastica, superficial spreading adenocarcinoma, adenocarcinoma, intestinal type, carcinoma, diffuse type, monomorphic adenoma, basal cell adenoma, islet cell adenoma, islet cell carcinoma, insulinoma nos, insulinoma, malignant, glucagonoma nos, glucagonoma, malignant, gastrinoma nos, gastrinoma, malignant, mixed islet cell and exocrine adenocarcinoma, bile duct adenoma, cholangiocarcinoma, bile duct cystadenoma, bile duct cystadenocarcinoma, liver cell adenoma, hepatocellular carcinoma nos, hepatocholangioma, benign, combined hepatocellular carcinoma and cholangiocarcinoma, trabecular adenoma, trabecular adenocarcinoma, embryonal adenoma, eccrine dermal cylindroma, adenoid cystic carcinoma, cribriform carcinoma, adenomatous polyp nos, adenocarcinoma in adenomatous polyp, tubular adenoma nos, tubular adenocarcinoma, adenomatous polyposis coli, adenocarcinoma in adenomatous polyposis coli, multiple adenomatous polyps, solid carcinoma nos, carcinoma simplex, carcinoid tumor nos, carcinoid tumor, malignant, carcinoid tumor, argentaffin nos, carcinoid tumor, argentaffin, malignant, carcinoid tumor, nonargentaffin nos, carcinoid tumor, nonargentaffin, malignant, mucocarcinoid tumor, malignant, composite carcinoid, pulmonary adenomatosis, bronchiolo-alveolar adenocarcinoma, alveolar adenoma, alveolar adenocarcinoma, papillary adenoma nos, papillary adenocarcinoma nos, villous adenoma nos, adenocarcinoma in villous adenoma, villous adenocarcinoma, tubulovillous adenoma, chromophobe adenoma, chromophobe carcinoma, acidophil adenoma, acidophil carcinoma, mixed acidophil-basophil adenoma, mixed acidophil-basophil carcinoma, oxyphilic adenoma, oxyphilic adenocarcinoma, basophil adenoma, basophil carcinoma, clear cell adenoma, clear cell adenocarcinoma nos, hypernephroid tumor, renal cell carcinoma, clear cell adenofibroma, granular cell carcinoma, chief cell adenoma, water-clear cell adenoma, water-clear cell adenocarcinoma, mixed cell adenoma, mixed cell adenocarcinoma, lipoadenoma, follicular adenoma, follicular adenocarcinoma nos, follicular adenocarcinoma, well differentiated type, follicular adenocarcinoma, trabecular type, microfollicular adenoma, macrofollicular adenoma, papillary and follicular adenocarcinoma, nonencapsulated sclerosing carcinoma, multiple endocrine adenomas, juxtaglomerular tumor, adrenal cortical adenoma nos, adrenal cortical carcinoma, adrenal cortical adenoma, compact cell type, adrenal cortical adenoma, heavily pigmented variant, adrenal cortical adenoma, clear cell type, adrenal cortical adenoma, glomerulosa cell type, adrenal cortical adenoma, mixed cell type, endometrioid adenoma nos, endometrioid adenoma, borderline malignancy, endometrioid carcinoma, endometrioid adenofibroma nos, endometrioid adenofibroma, borderline malignancy, endometrioid adenofibroma, malignant, adnexal and skin appendage neoplasms, skin appendage adenoma, skin appendage carcinoma, sweat gland adenoma, sweat gland tumor nos, sweat gland adenocarcinoma, apocrine adenoma, apocrine adenocarcinoma, eccrine acrospiroma, eccrine spiradenoma, hidrocystoma, papillary hydradenoma, papillary syringadenoma, syringoma nos, sebaceous adenoma, sebaceous adenocarcinoma, ceruminous adenoma, ceruminous adenocarcinoma, mucoepidermoid neoplasms, mucoepidermoid tumor, mucoepidermoid carcinoma, cystic, mucinous, and serous neoplasms, cystadenoma nos, cystadenocarcinoma nos, serous cystadenoma nos, serous cystadenoma, borderline malignancy, serous cystadenocarcinoma nos, papillary cystadenoma nos, papillary cystadenoma, borderline malignancy, papillary cystadenocarcinoma nos, papillary serous cystadenoma nos, papillary serous cystadenoma, borderline malignancy, papillary serous cystadenocarcinoma, serous surface papilloma nos, serous surface papilloma, borderline malignancy, serous surface papillary carcinoma, mucinous cystadenoma nos, mucinous cystadenoma, borderline malignancy, mucinous cystadenocarcinoma nos, papillary mucinous cystadenoma nos, papillary mucinous cystadenoma, borderline malignancy, papillary mucinous cystadenocarcinoma, mucinous adenoma, mucinous adenocarcinoma, pseudomyxoma peritonei, mucin-producing adenocarcinoma, signet ring cell carcinoma, metastatic signet ring cell carcinoma, ductal, lobular, and medullary neoplasms, intraductal carcinoma, noninfiltrating nos, infiltrating duct carcinoma, comedocarcinoma, noninfiltrating, comedocarcinoma nos, juvenile carcinoma of the breast, intraductal papilloma, noninfiltrating intraductal papillary adenocarcinoma, intracystic papillary adenoma, noninfiltrating intracystic carcinoma, intraductal papillomatosis nos, subareolar duct papillomatosis, medullary carcinoma nos, medullary carcinoma with amyloid stroma, medullary carcinoma with lymphoid stroma, lobular carcinoma in situ, lobular carcinoma nos, infiltrating ductular carcinoma, inflammatory carcinoma, paget's disease, mammary, paget's disease and infiltrating duct carcinoma of breast, paget's disease, extramammary, acinar cell neoplasms, acinar cell adenoma, acinar cell tumor, acinar cell carcinoma, complex epithelial neoplasms, adenosquamous carcinoma, adenolymphoma, adenocarcinoma with squamous metaplasia, adenocarcinoma with cartilaginous and osseous metaplasia, adenocarcinoma with spindle cell metaplasia, adenocarcinoma with apocrine metaplasia, thymoma, benign, thymoma, malignant, specialized gonadal neoplasms, sex cord-stromal tumor, thecoma nos, theca cell carcinoma, luteoma nos, granulosa cell tumor nos, granulosa cell tumor, malignant, granulosa cell-theca cell tumor, androblastoma, benign, androblastoma nos, androblastoma, malignant, sertoli-leydig cell tumor, gynandroblastoma, tubular androblastoma nos, sertoli cell carcinoma, tubular androblastoma with lipid storage, leydig cell tumor, benign, leydig cell tumor nos, leydig cell tumor, malignant, hilar cell tumor, lipid cell tumor of ovary, adrenal rest tumor, paragangliomas and glomus tumors, paraganglioma nos, paraganglioma, malignant, sympathetic paraganglioma, parasympathetic paraganglioma, glomus jugulare tumor, aortic body tumor, carotid body tumor, extra-adrenal paraganglioma nos, extra-adrenal paraganglioma, malignant, pheochromocytoma nos, pheochromocytoma, malignant, glomangiosarcoma, glomus tumor, glomangioma, nevi and melanomas, pigmented nevus nos, malignant melanoma nos, nodular melanoma, balloon cell nevus, balloon cell melanoma, halo nevus, fibrous papule of the nose, neuronevus, magnocellular nevus, nonpigmented nevus, amelanotic melanoma, junctional nevus, malignant melanoma in junctional nevus, precancerous melanosis nos, malignant melanoma in precancerous melanosis, hutchinson's melanotic freckle, malignant melanoma in hutchinson's melanotic freckle, superficial spreading melanoma, intradermal nevus, compound nevus, giant pigmented nevus, malignant melanoma in giant pigmented nevus, epithelioid and spindle cell nevus, epithelioid cell melanoma, spindle cell melanoma nos, spindle cell melanoma, type a, spindle cell melanoma, type b, mixed epithelioid and spindle cell melanoma, blue nevus nos, blue nevus, malignant, cellular blue nevus, soft tissue tumors and sarcomas nos, soft tissue tumor, benign, sarcoma nos, sarcomatosis nos, spindle cell sarcoma, giant cell sarcoma, small cell sarcoma, epithelioid cell sarcoma, fibromatous neoplasms, fibroma nos, fibrosarcoma nos, fibromyxoma, fibromyxosarcoma, periosteal fibroma, periosteal fibrosarcoma, fascial fibroma, fascial fibrosarcoma, infantile fibrosarcoma, elastofibroma, aggressive fibromatosis, abdominal fibromatosis, desmoplastic fibroma, fibrous histiocytoma nos, atypical fibrous histiocytoma, fibrous histiocytoma, malignant, fibroxanthoma nos, atypical fibroxanthoma, fibroxanthoma, malignant, dermatofibroma nos, dermatofibroma protuberans, dermatofibrosarcoma nos, myxomatous neoplasms, myxoma nos, myxosarcoma, lipomatous neoplasms, lipoma nos, liposarcoma nos, fibrolipoma, liposarcoma, well differentiated type, fibromyxolipoma, myxoid liposarcoma, round cell liposarcoma, pleomorphic liposarcoma, mixed type liposarcoma, intramuscular lipoma, spindle cell lipoma, angiomyolipoma, angiomyoliposarcoma, angiolipoma nos, angiolipoma, infiltrating, myelolipoma, hibernoma, lipoblastomatosis, myomatous neoplasms, leiomyoma nos, intravascular leiomyomatosis, leiomyosarcoma nos, epithelioid leiomyoma, epithelioid leiomyosarcoma, cellular leiomyoma, bizarre leiomyoma, angiomyoma, angiomyosarcoma, myoma, myosarcoma, rhabdomyoma nos, rhabdomyosarcoma nos, pleomorphic rhabdomyosarcoma, mixed type rhabdomyosarcoma, fetal rhabdomyoma, adult rhabdomyoma, embryonal rhabdomyosarcoma, alveolar rhabdomyosarcoma, complex mixed and stromal neoplasms, endometrial stromal sarcoma, endolymphatic stromal myosis, adenomyoma, pleomorphic adenoma, mixed tumor, malignant nos, mullerian mixed tumor, mesodermal mixed tumor, mesoblastic nephroma, nephroblastoma nos, epithelial nephroblastoma, mesenchymal nephroblastoma, hepatoblastoma, carcinosarcoma nos, carcinosarcoma, embryonal type, myoepithelioma, mesenchymoma, benign, mesenchymoma nos, mesenchymoma, malignant, embryonal sarcoma, fibroepithelial neoplasms, brenner tumor nos, brenner tumor, borderline malignancy, brenner tumor, malignant, fibroadenoma nos, intracanalicular fibroadenoma nos, pericanalicular fibroadenoma, adenofibroma nos, serous adenofibroma, mucinous adenofibroma, cellular intracanalicular fibroadenoma, cystosarcoma phyllodes nos, cystosarcoma phyllodes, malignant, juvenile fibroadenoma, synovial neoplasms, synovioma, benign, synovial sarcoma nos, synovial sarcoma, spindle cell type, synovial sarcoma, epithelioid cell type, synovial sarcoma, biphasic type, clear cell sarcoma of tendons and aponeuroses, mesothelial neoplasms, mesothelioma, benign, mesothelioma, malignant, fibrous mesothelioma, benign, fibrous mesothelioma, malignant, epithelioid mesothelioma, benign, epithelioid mesothelioma, malignant, mesothelioma, biphasic type, benign, mesothelioma, biphasic type, malignant, adenomatoid tumor nos, germ cell neoplasms, dysgerminoma, seminoma nos, seminoma, anaplastic type, spermatocytic seminoma, germinoma, embryonal carcinoma nos, endodermal sinus tumor, polyembryoma, gonadoblastoma, teratoma, benign, teratoma nos, teratoma, malignant nos, teratocarcinoma, malignant teratoma, undifferentiated type, malignant teratoma, intermediate type, dermoid cyst, dermoid cyst with malignant transformation, struma ovarii nos, struma ovarii, malignant, strumal carcinoid, trophoblastic neoplasms, hydatidiform mole nos, invasive hydatidiform mole, choriocarcinoma, choriocarcinoma combined with teratoma, malignant teratoma, trophoblastic, mesonephromas, mesonephroma, benign, mesonephric tumor, mesonephroma, malignant, endosalpingioma, blood vessel tumors, hemangioma nos, hemangiosarcoma, cavernous hemangioma, venous hemangioma, racemose hemangioma, kupffer cell sarcoma, hemangioendothelioma, benign, hemangioendothelioma nos, hemangioendothelioma, malignant, capillary hemangioma, intramuscular hemangioma, kaposi's sarcoma, angiokeratoma, verrucous keratotic hemangioma, hemangiopericytoma, benign, hemangiopericytoma nos, hemangiopericytoma, malignant, angiofibroma nos, hemangioblastoma, lymphatic vessel tumors, lymphangioma nos, lymphangiosarcoma, capillary lymphangioma, cavernous lymphangioma, cystic lymphangioma, lymphangiomyoma, lymphangiomyomatosis, hemolymphangioma, osteomas and osteosarcomas, osteoma nos, osteosarcoma nos, chondroblastic osteosarcoma, fibroblastic osteosarcoma, telangiectatic osteosarcoma, osteosarcoma in paget's disease of bone, juxtacortical osteosarcoma, osteoid osteoma nos, osteoblastoma, chondromatous neoplasms, osteochondroma, osteochondromatosis nos, chondroma nos, chondromatosis nos, chondrosarcoma nos, juxtacortical chondroma, juxtacortical chondrosarcoma, chondroblastoma nos, chondroblastoma, malignant, mesenchymal chondrosarcoma, chondromyxoid fibroma, giant cell tumors, giant cell tumor of bone nos, giant cell tumor of bone, malignant, giant cell tumor of soft parts nos, malignant giant cell tumor of soft parts, miscellaneous bone tumors, ewing's sarcoma, adamantinoma of long bones, ossifying fibroma, odontogenic tumors, odontogenic tumor, benign, odontogenic tumor nos, odontogenic tumor, malignant, dentinoma, cementoma nos, cementoblastoma, benign, cementifying fibroma, gigantiform cementoma, odontoma nos, compound odontoma, complex odontoma, ameloblastic fibro-odontoma, ameloblastic odontosarcoma, adenomatoid odontogenic tumor, calcifying odontogenic cyst, ameloblastoma nos, ameloblastoma, malignant, odontoameloblastoma, squamous odontogenic tumor, odontogenic myxoma, odontogenic fibroma nos, ameloblastic fibroma, ameloblastic fibrosarcoma, calcifying epithelial odontogenic tumor, miscellaneous tumors, craniopharyngioma, pinealoma, pineocytoma, pineoblastoma, melanotic neuroectodermal tumor, chordoma, gliomas, glioma, malignant, gliomatosis cerebri, mixed glioma, subependymal glioma, subependymal giant cell astrocytoma, choroid plexus papilloma nos, choroid plexus papilloma, malignant, ependymoma nos, ependymoma, anaplastic type, papillary ependymoma, myxopapillary ependymoma, astrocytoma nos, astrocytoma, anaplastic type, protoplasmic astrocytoma, gemistocytic astrocytoma, fibrillary astrocytoma, pilocytic astrocytoma, spongioblastoma nos, spongioblastoma polare, astroblastoma, glioblastoma nos, giant cell glioblastoma, glioblastoma with sarcomatous component, primitive polar spongioblastoma, oligodendroglioma nos, oligodendroglioma, anaplastic type, oligodendroblastoma, medulloblastoma nos, desmoplastic medulloblastoma, medullomyoblastoma, cerebellar sarcoma nos, monstrocellular sarcoma, neuroepitheliomatous neoplasms, ganglioneuroma, ganglioneuroblastoma, ganglioneuromatosis, neuroblastoma nos, medulloepithelioma nos, teratoid medulloepithelioma, neuroepithelioma nos, spongioneuroblastoma, ganglioglioma, neurocytoma, pacinian tumor, retinoblastoma nos, retinoblastoma, differentiated type, retinoblastoma, undifferentiated type, olfactory neurogenic tumor, esthesioneurocytoma, esthesioneuroblastoma, esthesioneuroepithelioma, meningiomas, meningioma nos, meningiomatosis nos, meningioma, malignant, meningotheliomatous meningioma, fibrous meningioma, psammomatous meningioma, angiomatous meningioma, hemangioblastic meningioma, hemangiopericytic meningioma, transitional meningioma, papillary meningioma, meningeal sarcomatosis, nerve sheath tumor, neurofibroma nos, neurofibromatosis nos, neurofibrosarcoma, melanotic neurofibroma, plexiform neurofibroma, neurilemmoma nos, neurinomatosis, neurilemmoma, malignant, neuroma nos, granular cell tumors and alveolar soft part sarcoma, granular cell tumor nos, granular cell tumor, malignant, alveolar soft part sarcoma, lymphomas, nos or diffuse, lymphomatous tumor, benign, malignant lymphoma nos, malignant lymphoma, non-hodgkin's type, malignant lymphoma, undifferentiated cell type nos, malignant lymphoma, stem cell type, malignant lymphoma, convoluted cell type nos, lymphosarcoma nos, malignant lymphoma, lymphoplasmacytoid type, malignant lymphoma, immunoblastic type, malignant lymphoma, mixed lymphocytic-histiocytic nos, malignant lymphoma, centroblastic-centrocytic, diffuse, malignant lymphoma, follicular center cell nos, malignant lymphoma, lymphocytic, well differentiated nos, malignant lymphoma, lymphocytic, intermediate differentiation nos, malignant lymphoma, centrocytic, malignant lymphoma, follicular center cell, cleaved nos, malignant lymphoma, lymphocytic, poorly differentiated nos, prolymphocytic lymphosarcoma, malignant lymphoma, centroblastic type nos, malignant lymphoma, follicular center cell, noncleaved nos, reticulosarcomas, reticulosarcoma nos, reticulosarcoma, pleomorphic cell type, reticulosarcoma, nodular, hodgkin's disease, hodgkin's disease nos, hodgkin's disease, lymphocytic predominance, hodgkin's disease, mixed cellularity, hodgkin's disease, lymphocytic depletion nos, hodgkin's disease, lymphocytic depletion, diffuse fibrosis, hodgkin's disease, lymphocytic depletion, reticular type, hodgkin's disease, nodular sclerosis nos, hodgkin's disease, nodular sclerosis, cellular phase, hodgkin's paragranuloma, hodgkin's granuloma, hodgkin's sarcoma, lymphomas, nodular or follicular, malignant lymphoma, nodular nos, malignant lymphoma, mixed lymphocytic-histiocytic, nodular, malignant lymphoma, centroblastic-centrocytic, follicular, malignant lymphoma, lymphocytic, well differentiated, nodular, malignant lymphoma, lymphocytic, intermediate differentiation, nodular, malignant lymphoma, follicular center cell, cleaved, follicular, malignant lymphoma, lymphocytic, poorly differentiated, nodular, malignant lymphoma, centroblastic type, follicular, malignant lymphoma, follicular center cell, noncleaved, follicular, mycosis fungoides, mycosis fungoides, sezary's disease, miscellaneous reticuloendothelial neoplasms, microglioma, malignant histiocytosis, histiocytic medullary reticulosis, letterer-siwe's disease, plasma cell tumors, plasma cell myeloma, plasma cell tumor, benign, plasmacytoma nos, plasma cell tumor, malignant, mast cell tumors, mastocytoma nos, mast cell sarcoma, malignant mastocytosis, burkitt's tumor, burkitt's tumor, leukemias, leukemias nos, leukemia nos, acute leukemia nos, subacute leukemia nos, chronic leukemia nos, aleukemic leukemia nos, compound leukemias, compound leukemia, lymphoid leukemias, lymphoid leukemia nos, acute lymphoid leukemia, subacute lymphoid leukemia, chronic lymphoid leukemia, aleukemic lymphoid leukemia, prolymphocytic leukemia, plasma cell leukemias, plasma cell leukemia, erythroleukemias, erythroleukemia, acute erythremia, chronic erythremia, lymphosarcoma cell leukemias, lymphosarcoma cell leukemia, myeloid leukemias, myeloid leukemia nos, acute myeloid leukemia, subacute myeloid leukemia, chronic myeloid leukemia, aleukemic myeloid leukemia, neutrophilic leukemia, acute promyelocytic leukemia, basophilic leukemias, basophilic leukemia, eosinophilic leukemias, eosinophilic leukemia, monocytic leukemias, monocytic leukemia nos, acute monocytic leukemia, subacute monocytic leukemia, chronic monocytic leukemia, aleukemic monocytic leukemia, miscellaneous leukemias, mast cell leukemia, megakaryocytic leukemia, megakaryocytic myelosis, myeloid sarcoma, hairy cell leukemia, miscellaneous myeloproliferative and lymphoproliferative disorders, polycythemia vera, acute panmyelosis, chronic myeloproliferative disease, myelosclerosis with myeloid metaplasia, idiopathic thrombocythemia, chronic lymphoproliferative disease.

In an embodiment of the present invention, the disease is selected from the group comprising tumors of pancreas, pancreatic adenocarcinoma, pancreatic ductal adenocarcinoma, tumors of head of pancreas, of body of pancreas, of tail of pancreas, of pancreatic duct, of islets of langerhans, neck of pancreas, tumor of prostate, prostate adenocarcinoma, prostate gland, neuroendocrine tumors, brain cancer, breast cancer, tumor of central portion of breast, upper inner quadrant of breast, lower inner quadrant of breast, upper outer quadrant of breast, lower outer quadrant of breast, axillary tail of breast, overlapping lesion of breast, juvenile carcinoma of the breast, tumors of parathyroid gland, myeloma, lung cancer, small cell lung cancer, non-small cell lung cancer including, but not limited to, squamous non-small cell lung cancer (Sq. NSCLC), tumor of main bronchus, of upper lobe lung, of middle lobe lung, of lower lobe lung, colorectal carcinoma, tumor of ascending colon, of hepatic flexure of colon, of transverse colon, of splenic flexure of colon, of descending colon, of sigmoid colon, of overlapping lesion of colon, of small intestine, tumors of liver, liver cell adenoma, hepatocellular carcinoma, hepatocholangioma, cholangiocarcinoma, combined hepatocellular carcinoma and cholangiocarcinoma, hepatoblastoma, ovarian carcinoma, sarcoma, osteosarcoma, fibrosarcoma, gastrointestinal stroma tumors, gastrointestinal tract, gastric carcinoma, thyroid carcinoma, medullary thyroid carcinoma, thyroid gland, renal cell carcinoma, clear cell renal cell carcinoma, renal pelvis, tumors of bladder, bladder carcinoma, tumors of trigone bladder, of dome bladder, of lateral wall bladder, of posterior wall bladder, of ureteric orifice, of urachus, overlapping lesion of bladder, basal cell carcinoma, basal cell neoplasms, basal cell tumor, basal cell carcinoma, multicentric basal cell carcinoma, basaloid carcinoma, basal cell adenoma, squamous cell carcinoma, oral squamous cell carcinoma, squamous cell carcinoma of the larynx, cervical carcinoma, tumors of exocervix, of overlapping lesion of cervix uteri, of cervix uteri, of isthmus uteri, tumors of uterus, tumors of ovary, tumors of cervical esophagus, of thoracic esophagus, of abdominal esophagus, of upper third of esophagus, of esophagus middle third, of esophagus lower third, of overlapping lesion of esophagus, endometrial carcinoma, head and neck cancer including, but not limited to, squamous cell carcinoma of head and neck (SCCHN), lymphoma, malignant mesothelioma, mesothelial neoplasms, mesothelioma, fibrous mesothelioma, fibrous mesothelioma, epithelioid mesothelioma, epithelioid mesothelioma, duodenal carcinoma, neuroendocrine tumors, neuroendocrine tumors of the lung, neuroendocrine tumors of the pancreas, neuroendocrine tumors of the foregut, neuroendocrine tumors of the midgut, neuroendocrine tumors of the hindgut, gastroenteropancreatic neuroendocrine tumors, neuroendocrine carcinomas, neuroendocrine tumors of the breast including, but not limited to, triple-negative breast cancer (TNBC), neuroendocrine tumors of the ovaries, testicular cancer, thymic carcinoma, tumors of stomach, fundus stomach, body stomach, gastric antrum, pylorus, lesser curvature of stomach, greater curvature of stomach, overlapping lesion of stomach, paragangliomas, ganglioma, melanomas, malignant melanoma, nodular melanoma, amelanotic melanoma, superficial spreading melanoma, epithelioid cell melanoma, spindle cell melanoma, mixed epithelioid and spindle cell melanoma, glioblastoma nos, giant cell glioblastoma, glioblastoma with sarcomatous component.

In an embodiment of the present invention, the disease is selected from the group comprising or consisting of non-small cell lung cancer including Sq. NSCLC, head and neck cancer including SCCHN, and neuroendocrine tumors of the breast including TNBC. Preferably, the disease is selected from the group comprising or consisting of Sq. NSCLC, SCCHN and TNBC.

In a still further embodiment, the aforementioned indications may occur in organs and tissues selected from the group comprising external upper lip, external lower lip, external lip nos, upper lip mucosa, lower lip mucosa, mucosa lip nos, commissure lip, overlapping lesion of lip, base of tongue nos, dorsal surface tongue nos, border of tongue, ventral surface of tongue nos, anterior ⅔ of tongue nos, lingual tonsil, overlapping lesion of tongue, tongue nos, upper gum, lower gum, gum nos, anterior floor of mouth, lateral floor of mouth, overlapping lesion of floor of mouth, floor of mouth nos, hard palate, soft palate nos, uvula, overlapping lesion of palate, palate nos, cheek mucosa, vestibule of mouth, retromolar area, overlapping lesion of other and unspecified parts of mouth, mouth nos, parotid gland, submaxillary gland, sublingual gland, overlapping lesion of major salivary glands, major salivary gland nos, tonsillar fossa, tonsillar pillar, overlapping lesion of tonsil, tonsil nos, vallecula, anterior surface of epiglottis, lateral wall oropharynx, posterior wall oropharynx, branchial cleft, overlapping lesion of oropharynx, oropharynx nos, superior wall of nasopharynx, posterior wall nasopharynx, lateral wall nasopharynx, anterior wall nasopharynx, overlapping lesion of nasopharynx, nasopharynx nos, pyriform sinus, postcricoid region, hypopharyngeal aspect of aryepiglottic fold, posterior wall hypopharynx, overlapping lesion of hypopharynx, hypopharynx nos, pharynx nos, laryngopharynx, waldeyer's ring, overlapping lesion of lip oral cavity and pharynx, cervical esophagus, thoracic esophagus, abdominal esophagus, upper third of esophagus, middle third of esophagus, esophagus lower third, overlapping lesion of esophagus, esophagus nos, cardia nos, fundus stomach, body stomach, gastric antrum, pylorus, lesser curvature of stomach nos, greater curvature of stomach nos, overlapping lesion of stomach, stomach nos, duodenum, jejunum, ileum, meckel's diverticulum, overlapping lesion of small intestine, small intestine nos, cecum, appendix, ascending colon, hepatic flexure of colon, transverse colon, splenic flexure of colon, descending colon, sigmoid colon, overlapping lesion of colon, colon nos, rectosigmoid junction, rectum nos, anus nos, anal canal, cloacogenic zone, overlapping lesion of rectum anus and anal canal, liver, intrahepatic bile duct, gallbladder, extrahepatic bile duct, ampulla of vater, overlapping lesion of biliary tract, biliary tract nos, head of pancreas, body pancreas, tail pancreas, pancreatic duct, islets of langerhans, neck of pancreas, overlapping lesion of pancreas, pancreas nos, intestinal tract nos, overlapping lesion of digestive system, gastrointestinal tract nos, nasal cavity, middle ear, maxillary sinus, ethmoid sinus, frontal sinus, sphenoid sinus, overlapping lesion of accessory sinuses, accessory sinus nos, glottis, supraglottis, subglottis, laryngeal cartilage, overlapping lesion of larynx, larynx nos, trachea, main bronchus, upper lobe lung, middle lobe lung, lower lobe lung, overlapping lesion of lung, lung nos, thymus, heart, anterior mediastinum, posterior mediastinum, mediastinum nos, pleura nos, overlapping lesion of heart mediastinum and pleura, upper respiratory tract nos, overlapping lesion of respiratory system and intrathoracic organs, respiratory tract nos, upper limb long bones joints, upper limb short bones joints, lower limb long bones joints, lower limb short bones joints, overlapping lesion of bones joints and articular cartilage of limbs, bone limb nos, skull and facial bone, mandible, vertebral column, rib sternum clavicle, pelvic bone, overlapping lesion of bones joints and articular cartilage, bone nos, blood, bone marrow, spleen, reticuloendothelial system nos, hematopoietic system nos, skin lip nos, eyelid nos, external ear, skin face, skin scalp neck, skin trunk, skin limb upper, skin limb lower, peripheral nerve head neck, peripheral nerve shoulder arm, peripheral nerve leg, peripheral nerve thorax, peripheral nerve abdomen, peripheral nerve pelvis, peripheral nerve trunk, overlapping lesion of peripheral nerves and autonomic nervous system, autonomic nervous system nos, retroperitoneum, peritoneum, peritoneum nos, overlapping lesion of retroperitoneum and peritoneum, connective tissue head, connective tissue arm, connective tissue leg, connective tissue thorax, connective tissue abdomen, connective tissue pelvis, connective tissue trunk nos, overlapping lesion of connective subcutaneous and other soft tissues, connective tissue nos, nipple, central portion of breast, upper inner quadrant of breast, lower inner quadrant of breast, upper outer quadrant of breast, lower outer quadrant of breast, axillary tail of breast, overlapping lesion of breast, breast nos, labium majus, labium minus, clitoris, overlapping lesion of vulva, vulva nos, vagina nos, endocervix, exocervix, overlapping lesion of cervix uteri, cervix uteri, isthmus uteri, endometrium, myometrium, fundus uteri, overlapping lesion of corpus uteri, corpus uteri, uterus nos, ovary, fallopian tube, broad ligament, round ligament, parametrium, uterine adnexa, wolffian body, overlapping lesion of female genital organs, female genital tract nos, prepuce, glans penis, body penis, overlapping lesion of penis, penis nos, prostate gland, undescended testis, descended testis, testis nos, epididymis, spermatic cord, scrotum nos, tunica vaginalis, overlapping lesion of male genital organs, male genital organs nos, kidney nos, renal pelvis, ureter, trigone bladder, dome bladder, lateral wall bladder, posterior wall bladder, ureteric orifice, urachus, overlapping lesion of bladder, bladder nos, urethra, paraurethral gland, overlapping lesion of urinary organs, urinary system nos, conjunctiva, cornea nos, retina, choroid, ciliary body, lacrimal gland, orbit nos, overlapping lesion of eye and adnexa, eye nos, cerebral meninges, spinal meninges, meninges nos, cerebrum, frontal lobe, temporal lobe, parietal lobe, occipital lobe, ventricle nos, cerebellum nos, brain stem, overlapping lesion of brain, brain nos, spinal cord, cauda equina, olfactory nerve, optic nerve, acoustic nerve, cranial nerve nos, overlapping lesion of brain and central nervous system, nervous system nos, thyroid gland, adrenal gland cortex, adrenal gland medulla, adrenal gland nos, parathyroid gland, pituitary gland, craniopharyngeal duct, pineal gland, carotid body, aortic body, overlapping lesion of endocrine glands and related structures, endocrine gland nos, head face or neck nos, thorax nos, abdomen nos, pelvis nos, upper limb nos, lower limb nos, other illdefined sites, overlapping lesion of ill-defined sites, lymph node face head neck, intrathoracic lymph node, intra-abdominal lymph nodes, lymph node axilla arm, lymph node inguinal region leg, lymph node pelvic, lymph nodes of multiple regions, lymph node nos, unknown primary site.

In an embodiment of the present invention, the cancers listed herein are locally advanced, unresectable, metastatic, or any combination thereof.

In an embodiment, the compound of the invention is used or is for use in a method for the treatment of a cancer associated with an alteration of the von Hippel-Lindau (VHL) gene. The VHL gene is a tumor suppressor gene, which may be inactivated by genetic alteration including, e.g., VHL mutation, promoter hypermethylation, and loss of heterozygosity by allele deletion. Inactivation of VHL has been associated with increased tumorigenesis and progression, and especially with increased renal tumorigenesis and progression (Wiesener et al. Cancer Res. 2001, 61, 215-222). Furthermore, VHL mutations have been reportedly associated with high levels of CAIX expression, whereas the absence of VHL mutation has been associated with low CAIX expression and aggressive tumor characteristics (Pantuck et al. Journal of Clinical Oncology 2007, 25(18), 5042; Patard et al. Int J Cancer 2008, 123(2), 395-400). In an embodiment, the cancer is associated with a mutation of the VHL gene.

The terms “an alteration” and “a mutation” as used above are to be understood as encompassing single as well as multiple alterations and mutations, respectively, i.e., as “one or more alterations” and “one or more mutations”, respectively.

Tumor profiling can be performed by extracting DNA from the formalin-fixed, paraffin embedded (FFPE) tissue from cancer patients and determining the alteration(s) of the von Hippel-Lindau (VHL) gene by means of known gene sequencing techniques. In some aspects, VHL mutations can be identified by bi-directional sequencing analysis of all exons and short adjacent intronic sequences. Large genomic and intragenic deletions may be identified by Southern blotting, including quantitative Southern blotting, pulsed field gel electrophoresis and/or fluorescence in situ hybridization, quantitative real-time PCR (Q-RT-PCR), multiplex ligation-dependent probe amplification (MLPA), or comparative genomic hybridization (CGH) (Decker et al. European Journal of Human Genetics 2014, 22). Preferably, VHL mutations can be identified by sequencing followed by MLPA. In some aspects, tumor profiling as described above can be used to predict the response of a patient diagnosed with cancer to treatment and/or imaging with the compound of the invention.

In a further embodiment, the compound of the invention is used or is for use in a method for the treatment of a cancer associated with an alteration of the von Hippel-Lindau (VHL) gene, wherein the cancer is selected from the group consisting of clear cell renal cell carcinoma (ccRCC), renal cell carcinoma (RCC), lung cancer, colorectal carcinoma (CRC), and bladder cancer.

In yet a further embodiment, the compound of the invention is used or is for use in a method for the treatment of a cancer associated with an alteration of the von Hippel-Lindau (VHL) gene, wherein the cancer is clear cell renal cell carcinoma (ccRCC).

The subjects treated with the compounds of the invention may be treated in combination with other non-surgical anti-proliferative (e.g., anti-cancer) drug therapy. In one embodiment, the compounds may be administered in combination with an anti-cancer compound such as a cytostatic compound. A cytostatic compound is a compound (e.g., a small molecule, a nucleic acid, or a protein) that suppresses cell growth and/or proliferation. In some embodiments, the cytostatic compound is directed towards the malignant cells of a tumor.

Suitable anti-proliferative drugs or cytostatic compounds to be used in combination with the compounds of the invention include anti-cancer drugs. Numerous anti-cancer drugs which may be used are well known and include, but are not limited to: Acivicin; Aclarubicin; Acodazole Hydrochloride; Acronine; Adozelesin; Aldesleukin; Altretamine; Ambomycin; Ametantrone Acetate; Aminoglutethimide; Amsacrine; Anastrozole; Anthramycin; Asparaginase; Asperlin; Azacitidine; Azetepa; Azotomycin; Batimastat; Benzodepa; Bicalutamide; Bisantrene Hydrochloride; Bisnafide Dimesylate; Bizelesin; Bleomycin Sulfate; Brequinar Sodium; Bropirimine; Busulfan; Cactinomycin; Calusterone; Caracemide; Carbetimer; Carboplatin; Carmustine; Carubicin Hydrochloride; Carzelesin; Cedefingol; Chlorambucil; Cirolemycin; Cisplatin; Cladribine; Crisnatol Mesylate; Cyclophosphamide; Cytarabine; Dacarbazine; Dactinomycin; Daunorubicin Hydrochloride; Decitabine; Dexormaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomycin; Edatrexate; Eflornithine Hydrochloride; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Etoposide; Etoposide Phosphate; Etoprine; Fadrozole Hydrochloride; Fazarabine; Fenretinide; Floxuridine; Fludarabine Phosphate; Fluorouracil; Fluorocitabine; Fosquidone; Fostriecin Sodium; Gemcitabine; Gemcitabine Hydrochloride; Hydroxyurea; Idarubicin Hydrochloride; Ifosfamide; Ilmofosine; Interferon Alfa-2a; Interferon Alfa-2b; Interferon Alfa-n1; Interferon Alfa-n3; Interferon Beta-I a; Interferon Gamma-I b; Iproplatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Metoprine; Meturedepa; Mitindomide; Mitocarcin; Mitocromin; Mitogillin; Mitomalcin; Mitomycin; Mitosper; Mitotane; Mitoxantrone Hydrochloride; Mycophenolic Acid; Niraparib; Nocodazole; Nogalamycin; Olaparib; Ormaplatin; Oxisuran; Paclitaxel; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rogletimide; Rucaparib; Safingol; Safingol Hydrochloride; Semustine; Simtrazene; Sparfosate Sodium; Sparsomycin; Spirogermanium Hydrochloride; Spiromustine; Spiroplatin; Streptonigrin; Streptozocin; Sulofenur; Talazoparib; Talisomycin; Taxol; Taxotere; Tecogalan Sodium; Tegafur; Teloxantrone Hydrochloride; Temoporfin; Teniposide; Teroxirone; Testolactone; Thiamiprine; Thioguanine; Thiotepa; Tiazofurin; Tirapazamine; Topotecan Hydrochloride; Toremifene Citrate; Trestolone Acetate; Triciribine Phosphate; Trimetrexate; Trimetrexate Glucuronate; Tubulozole Hydrochloride; Uracil Mustard; Uredepa; Vapreotide; Velaparib; Verteporfin; Vinblastine Sulfate; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; and Zorubicin Hydrochloride.

Other anti-cancer drugs include, but are not limited to: 20-epi-1,25 dihydroxyvitamin D3; 5-ethynyluracil; abiraterone; acylfulvene; adecypenol; adozelesin; ALL-TK antagonists; ambamustine; amidox; amifostine; aminolevulinic acid; amrubicin; anagrelide; andrographolide; angiogenesis inhibitors; antagonist D; antagonist G; antarelix; anti-dorsalizing morphogenetic protein-1; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bisaziridinylspermine; bisnafide; bistratene A; breflate; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; casein kinase inhibitors (ICOS); castanospermine; cecropin B; cetrorelix; chlorins; chloroquinoxaline sulfonamide; cicaprost; cis-porphyrin; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; dehydrodidemnin B; deslorelin; dexifosfamide; dexrazoxane; dexverapamil; didemnin B; didox; diethylnorspermine; dihydro-5-azacytidine; dihydrotaxol, 9-; dioxamycin; diphenyl spiromustine; docosanol; dolasetron; doxifluridine; dronabinol; duocarmycin SA; ebselen; ecomustine; edelfosine; edrecolomab; eflomithine; elemene; emitefur; epirubicin; epristeride; estramustine analogue; estrogen agonists; estrogen antagonists; etanidazole; etoposide phosphate; exemestane; filgrastim; finasteride; flavopiridol; flezelastine; fluasterone; fludarabine; fluorodaunorunicin hydrochloride; forfenimex; formestane; fotemustine; gadolinium texaphyrin; gallium nitrate; galocitabine; ganirelix; gelatinase inhibitors; glutathione inhibitors; hepsulfam; heregulin; hexamethylene bisacetamide; hypericin; ibandronic acid; idoxifene; idramantone; ilmofosine; ilomastat; imidazoacridones; imiquimod; immunostimulant peptides; insulin-like growth factor-I receptor inhibitor; interferon agonists; interferons; interleukins; iobenguane; iododoxorubicin; ipomeanol, 4-; irinotecan; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anti cancer compound; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; 06-benzylguanine; octreotide; okicenone; oligonucleotides; onapristone; ondansetron; ondansetron; oracin; oral cytokine inducer; osaterone; oxaliplatin; oxaunomycin; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rohitukine; romurtide; roquinimex; rubiginone B1; ruboxyl; saintopin; SarCNU; sarcophytol A; sargramostim; Sdi 1 mimetics; senescence derived inhibitor 1; sense oligonucleotides; signal transduction inhibitors; signal transduction modulators; single chain antigen binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temozolomide; tetrachlorodecaoxide; tetrazomine; thaliblastine; thalidomide; thiocoraline; thrombopoietin; thrombopoietin mimetic; thymalfasin; thymopoietin receptor agonist; thymotrinan; thyroid stimulating hormone; tin ethyl etiopurpurin; titanocene dichloride; topsentin; toremifene; totipotent stem cell factor; translation inhibitors; tretinoin; triacetyluridine; triciribine; tropisetron; turosteride; tyrosine kinase inhibitors; tyrphostins; UBC inhibitors; ubenimex; urogenital sinus-derived growth inhibitory factor; urokinase receptor antagonists; variolin B; vector system, erythrocyte gene therapy; velaresol; veramine; verdins; vinorelbine; vinxaltine; vitaxin; zanoterone; zilascorb; and zinostatin stimalamer.

The compounds of the present invention can also be used in combination with any of the following treatments:

Therapy in combination with inhibitors of Poly(ADP-ribose) polymerases (PARP), a class of chemotherapeutic agents directed at targeting cancers with defective DNA-damage repair (Yuan, et al., Expert Opin Ther Pat, 2017, 27: 363). Such PARP inhibitors include but are not limited to olaparib, rupacarib, velaparib, niraparib, talazoparib, pamiparib, iniparib, E7449, and A-966492.

Therapy in combination with inhibitors of signaling pathways and mechanisms leading to repair of DNA single and double strand breaks as e.g. nuclear factor-kappaB signaling (Pilie, et al., Nat Rev Clin Oncol, 2019, 16: 81; Zhang, et al., Chin J Cancer, 2012, 31: 359). Such inhibitors include but are not limited to inhibitors of ATM and ATR kinases, checkpoint kinase 1 and 2, DNA-dependen protein kinase, and WEEl kinase (Pilie, et al., Nat Rev Clin Oncol, 2019, 16: 81).

Therapy in combination with an immunomodulator (Khalil, et al., Nat Rev Clin Oncol, 2016, 13: 394), a cancer vaccine (Hollingsworth, et al., NPJ Vaccines, 2019, 4: 7), an immune checkpoint inhibitor (e.g. PD-1, PD-L1, CTLA-4-inhibitor) (Wei, et al., Cancer Discov, 2018, 8: 1069), a Cyclin-D-Kinase 4/6 inhibitor (Goel, et al., Trends Cell Biol, 2018, 28: 911), an antibody being capable of binding to a tumor cell and/or metastases and being capable of inducing antibody-dependent cellular cytotoxicity (ADCC) (Kellner, et al., Transfus Med Hemother, 2017, 44: 327), a T cell- or NK cell engager (e.g. bispecific antibodies) (Yu, et al., J Cancer Res Clin Oncol, 2019, 145: 941), a cellular therapy using expanded autologous or allogeneic immune cells (e.g. chimeric antigen receptor T (CAR-T) cells) (Khalil, et al., Nat Rev Clin Oncol, 2016, 13: 394). Immune checkpoint inhibitors incluce but are not limited to nivolumab, ipilimumab, pembrolizumab, atezolizumab, avelumab, durvalumab, and cemiplimab.

According to the present invention, the compounds may be administered prior to, concurrent with, or following other anti-cancer compounds. The administration schedule may involve administering the different agents in an alternating fashion. In other embodiments, the compounds may be delivered before and during, or during and after, or before and after treatment with other therapies. In some cases, the compound is administered more than 24 hours before the administration of the other anti-proliferative treatment. In other embodiments, more than one anti-proliferative therapy may be administered to a subject. For example, the subject may receive the present compounds, in combination with both surgery and at least one other anti-proliferative compound. Alternatively, the compound may be administered in combination with more than one anti-cancer drug.

In an embodiment, the compounds of the present invention are used to detect cells and tissues overexpressing CAIX, whereby such detection is achieved by conjugating a detectable label to the compounds of the invention, preferably a detectable radionuclide. In a preferred embodiment, the cells and tissues detected are diseased cells and tissues and/or are either a or the cause for the disease and/or the symptoms of the disease, or are part of the pathology underlying the disease. In a further preferred embodiment, the diseased cells and tissues are causing and/or are part of an oncology indication (e.g. neoplasms, tumors, and cancers).

In another embodiment, the compounds of the present invention are used to treat cells and tissues overexpressing CAIX. In a preferred embodiment, the cells and tissues treated are diseased cells and tissues and/or are either a or the cause for the disease and/or the symptoms of the disease, or are part of the pathology underlying the disease. In a further preferred embodiment, the diseased cells and tissues are causing and/or are part of an oncology indication (e.g. neoplasms, tumors, and cancers) and the therapeutic activity is achieved by conjugating therapeutically active effector to the compounds of the present invention, preferably a therapeutically active radionuclide.

An effective amount is a dosage of the compound sufficient to provide a therapeutically or medically desirable result or effect in the subject to which the compound is administered. The effective amount will vary with the particular condition being treated, the age and physical condition of the subject being treated, the severity of the condition, the duration of the treatment, the nature of the concurrent or combination therapy (if any), the specific route of administration and like factors within the knowledge and expertise of the health practitioner. For example, in connection with methods directed towards treating subjects having a condition characterized by abnormal cell proliferation, an effective amount to inhibit proliferation would be an amount sufficient to reduce or halt altogether the abnormal cell proliferation so as to slow or halt the development of or the progression of a cell mass such as, for example, a tumor. As used in the embodiments, “inhibit” embraces all of the foregoing.

In other embodiments, a therapeutically effective amount will be an amount necessary to extend the dormancy of micrometastases or to stabilize any residual primary tumor cells following surgical or drug therapy.

In a preferred embodiment, the compound of the present invention is for use in the treatment and/or prevention of a disease, whereby such treatment is targeted radionuclide therapy. Targeted radionuclide therapy is a form of radiation therapy (also called radiotherapy) using molecules labeled with a radionuclide to deliver a toxic level of radiation to sites of disease. Targeted radionuclide therapy may be applied systemically or locally. In contrast, in external beam radiation therapy a source outside of the body is producing a high-energy beam, which is then focused at sites of disease, passing through the skin into the body. It is as well distinguished from internal radiation therapy (brachytherapy), where a radioactive implant is placed at or near the site of disease.

Preferably, radionuclide therapy makes use of or is based on different forms of radiation emitted by a radionuclide. Such radiation can, for example, be any one of alpha (α), beta (β) or gamma (γ) radiation caused by the emission of photons, emission of electrons including but not limited to β⁻-particles and Auger-electrons, emission of protons, emission of neutrons, emission of positrons or emission of α-particles. Depending on the kind of particle or radiation emitted by said radionuclide, radionuclide therapy can, for example, be distinguished as β-particle radionuclide therapy, α-particle radionuclide therapy or Auger electron radionuclide therapy. All of these forms of radionuclide therapy are encompassed by the present invention, and all of these forms of radionuclide therapy can be realized by the compound of the invention, preferably under the proviso that the radionuclide attached to the compound of the invention, more preferably as an effector, is providing for this kind of radiation.

Radionuclide therapy preferably works by damaging the DNA of cells. The damage is caused by a β-particle, α-particle, or Auger electron directly or indirectly ionizing the atoms which make up the DNA chain. Indirect ionization happens as a result of the ionization of water, forming free radicals, notably hydroxyl radicals, which then damage the DNA.

In the most common forms of radionuclide therapy, most of the radiation effect is through free radicals. Because cells have mechanisms for repairing DNA damage, breaking the DNA on both strands proves to be the most significant technique in modifying cell characteristics. Because cancer cells generally are undifferentiated and stem cell-like, they reproduce more, and have a diminished ability to repair sub-lethal damage compared to most healthy differentiated cells. The DNA damage is inherited through cell division, accumulating damage to the cancer cells, causing them to die or reproduce more slowly.

Oxygen is a potent radiosensitizer, increasing the effectiveness of a given dose of radiation by forming DNA-damaging free radicals. Therefore, use of high-pressure oxygen tanks, blood substitutes that carry increased oxygen, hypoxic cell radiosensitizers such as misonidazole and metronidazole, and hypoxic cytotoxins, such as tirapazamine may be applied.

The total radioactive dose may be fractionated, i.e. spread out over time in one or more treatments for several important reasons. Fractionation allows normal cells time to recover, while tumor cells are generally less efficient in repair between fractions. Fractionation also allows tumor cells that were in a relatively radio-resistant phase of the cell cycle during one treatment to cycle into a sensitive phase of the cycle before the next fraction is given.

It is generally known that different cancers respond differently to radiation therapy. The response of a cancer to radiation is described by its radiosensitivity. Highly radiosensitive cancer cells are rapidly killed by modest doses of radiation. These include leukemias, most lymphomas, and germ cell tumors.

Radionuclide therapy is in itself painless. Many low-dose palliative treatments cause minimal or no side effects. Treatment to higher doses may cause varying side effects during treatment (acute side effects), in the months or years following treatment (long-term side effects), or after re-treatment (cumulative side effects). The nature, severity, and longevity of side effects depends on the organs that receive the radiation, the treatment itself (type of radionuclide, dose, fractionation, concurrent chemotherapy), and the patient.

It is within the present inventions that the method for the treatment of a disease of the invention may realize each and any of the above strategies which are as such known in the art, and which insofar constitute further embodiments of the invention.

It is also within the present invention that the compound of the invention is used in a method for the diagnosis of a disease as disclosed herein. Such method, preferably, comprises the step of administering to a subject in need thereof a diagnostically effective amount of the compound of the invention.

In accordance with the present invention, an imaging method is selected from the group consisting of scintigraphy, Single Photon Emission Computed Tomography (SPECT) and Positron Emission Tomography (PET).

Scintigraphy is a form of diagnostic test or method used in nuclear medicine, wherein radiopharmaceuticals are internalized by cells, tissues and/or organs, preferably internalized in vivo, and radiation emitted by said internalized radiopharmaceuticals is captured by external detectors (gamma cameras) to form and display two-dimensional images. In contrast thereto, SPECT and PET forms and displays three-dimensional images. Because of this, SPECT and PET are classified as separate techniques to scintigraphy, although they also use gamma cameras to detect internal radiation. Scintigraphy is unlike a diagnostic X-ray where external radiation is passed through the body to form an image.

Single Photon Emission Tomography (SPECT) scans are a type of nuclear imaging technique using gamma rays. They are very similar to conventional nuclear medicine planar imaging using a gamma camera. Before the SPECT scan, the patient is injected with a radiolabeled compound emitting gamma rays that can be detected by the scanner. A computer collects the information from the gamma camera and translates this into two-dimensional cross-sections. These cross-sections can be added back together to form a three-dimensional image of an organ or a tissue. SPECT involves detection of gamma rays emitted singly, and sequentially, by the radionuclide provided by the radiolabeled compound. To acquire SPECT images, the gamma camera is rotated around the patient. Projections are acquired at defined points during the rotation, typically every 3-6 degrees. In most cases, a full 360 degree rotation is used to obtain an optimal reconstruction. The time taken to obtain each projection is also variable, but 15-20 seconds is typical. This gives a total scan time of 15-20 minutes. Multi-headed gamma cameras are faster. Since SPECT acquisition is very similar to planar gamma camera imaging, the same radiopharmaceuticals may be used.

Positron Emitting Tomography (PET) is a non-invasive, diagnostic imaging technique for measuring the biochemical, physiological and pathophysiological processes within the human body. PET is unique since it is able to produce images of the body's basic biochemistry or functions. Traditional diagnostic techniques, such as X-rays, CT scans, or MRI, produce images of the body's anatomy or structure. The premise with these techniques is that any changes in structure or anatomy associated with a disease can be seen. Biochemical and physiological processes are also altered by a disease, and may occur before any gross changes in anatomy. PET is an imaging technique that can visualize some of these early biochemical and physiological changes. PET scanners rely on radiation emitted from the patient to create the images. Each patient is given a minute amount of a radioactive compound that either closely resembles a natural substance used by the body or binds specifically to a receptor or molecular structure. As the radioisotope undergoes positron emission decay (also known as positive beta decay), it emits a positron, the antiparticle counterpart of an electron. After traveling up to a few millimeters, the positron encounters an electron and annihilates, producing a pair of annihilation (gamma) photons moving in opposite directions. These are detected when they reach a scintillation material in the scanning device, creating a burst of light, which is detected by photomultiplier tubes or silicon avalanche photodiodes. The technique depends on simultaneous or coincident detection of the pair of photons. Photons that do not arrive in pairs, i.e., within a few nanoseconds, are ignored. All coincidences are forwarded to the image processing unit where the final image data is produced using image reconstruction procedures.

SPECT/CT and PET/CT is the combination of SPECT and PET with computed tomography (CT). The key benefits of combining these modalities are improving the reader's confidence and accuracy. With traditional PET and SPECT, the limited number of photons emitted from the area of abnormality produces a very low-level background that makes it difficult to anatomically localize the area. Adding CT helps determine the location of the abnormal area from an anatomic perspective and categorize the likelihood that this represents a disease.

It is within the present inventions that the method for the diagnosis of a disease of the invention may realize each and any of the above strategies which are as such known in the art, and which insofar constitute further embodiments of the invention.

The compound of the invention has a high binding affinity to CAIX. Because of this high binding affinity, the compound of the invention is effective as, useful as and/or suitable as a targeting agent and, if conjugated to another moiety, as a targeting moiety. As preferably used herein a targeting agent is an agent which interacts with the target molecule which is in the instant case said CAIX. In terms of cells and tissues thus targeted by the compound of the invention any cell and tissue, respectively, expressing said CAIX is or may be targeted.

In an embodiment, the compound interacts with a carbonic anhydrase IX (CAIX), preferably with human CAIX having an amino acid sequence of SEQ ID NO: 4 or a homolog thereof, wherein the amino acid sequence of the homolog has an identity of CAIX that is at least 85% to the amino acid sequence of SEQ ID NO: 4. In preferred embodiments, the identity is 90%, preferably 95%, 96%, 97%, 98% or 99%.

The identity between two nucleic acid molecules can be determined as known to the person skilled in the art. More specifically, a sequence comparison algorithm may be used for calculating the percent sequence homology for the test sequence(s) relative to the reference sequence, based on the designated program parameters. The test sequence is preferably the sequence or protein or polypeptide which is said to be identical or to be tested whether it is identical, and if so, to what extent, to a different protein or polypeptide, whereby such different protein or polypeptide is also referred to as the reference sequence and is preferably the protein or polypeptide of wild type, more preferably the human CAIX of SEQ ID NO: 4.

Optimal alignment of sequences for comparison can be conducted, e.g., by the local homology algorithm of Smith & Waterman (Smith, et al., Advances in Applied Mathematics, 1981, 2: 482), by the homology alignment algorithm of Needleman & Wunsch (Needleman, et al., J Mol Biol, 1970, 48: 443), by the search for similarity method of Pearson & Lipman (Pearson, et al., Proc Natl Acad Sci USA, 1988, 85: 2444), by computerized implementations of these algorithms (GAP, BESTFIT, FASTA, and TFASTA in the Wisconsin Genetics Software Package, Genetics Computer Group, 575 Science Dr., Madison, Wis.), or by visual inspection.

One example of an algorithm that is suitable for determining percent sequence identity is the algorithm used in the basic local alignment search tool (hereinafter “BLAST”), see, e.g. Altschul et al., 1990 (Altschul, et al., J Mol Biol, 1990, 215: 403) and Altschul et al., 1997 (Altschul, et al., Nucleic Acids Res, 1997, 25: 3389). Software for performing BLAST analyses is publicly available through the National Center for Biotechnology Information (hereinafter “NCBI”). The default parameters used in determining sequence identity using the software available from NCBI, e.g., BLASTN (for nucleotide sequences) and BLASTP (for amino acid sequences) are described in McGinnis et al. (McGinnis, et al., Nucleic Acids Res, 2004, 32: W20).

Compounds of the present invention are useful to stratify patients, i.e. to create subsets within a patient population that provide more detailed information about how the patient will respond to a given drug. Stratification can be a critical component to transforming a clinical trial from a negative or neutral outcome to one with a positive outcome by identifying the subset of the population most likely to respond to a novel therapy.

Stratification includes the identification of a group of patients with shared “biological” characteristics to select the optimal management for the patients and achieve the best possible outcome in terms of risk assessment, risk prevention and achievement of the optimal treatment outcome

A compound of the present invention may be used to assess or detect, a specific disease as early as possible (which is a diagnostic use), the risk of developing a disease (which is a susceptibility/risk use), the evolution of a disease including indolent vs. aggressive (which is a prognostic use) and it may be used to predict the response and the toxicity to a given treatment (which is a predictive use).

It is also within the present invention that the compound of the invention is used in a theragnostic method. The concept of theragnostics is to combine a therapeutic agent with a corresponding diagnostic test that can increase the clinical use of the therapeutic drug. The concept of theragnostics is becoming increasingly attractive and is widely considered the key to improving the efficiency of drug treatment by helping doctors identify patients who might profit from a given therapy and hence avoid unnecessary treatments.

The concept of theragnostics is to combine a therapeutic agent with a diagnostic test that allows doctors to identify those patients who will benefit most from a given therapy. In an embodiment and as preferably used herein, a compound of the present invention is used for the diagnosis of a patient, i.e. identification and localization of the primary tumor mass as well as potential local and distant metastases. Furthermore, the tumor volume can be determined, especially utilizing three-dimensional diagnostic modalities such as SPECT or PET. Only those patients having CAIX-positive tumor masses and who, therefore, might profit from a given therapy are selected for a particular therapy and hence unnecessary treatments are avoided. Preferably, such therapy is a CAIX-targeted therapy using a compound of the present invention. In one particular embodiment, chemically identical tumor-targeted diagnostics, preferably imaging diagnostics for scintigraphy, PET or SPECT and radiotherapeutics are applied. Such compounds only differ in the radionuclide and therefore usually have a very similar if not identical pharmacokinetic profile. This can be realized using a chelator and a diagnostic or therapeutic radiometal. Alternatively, this can be realized using a precursor for radiolabeling and radiolabeling with either a diagnostic or a therapeutic radionuclide. In one embodiment diagnostic imaging is used preferably by means of quantification of the radiation of the diagnostic radionuclide and subsequent dosimetry which is known to those skilled in the art and the prediction of drug concentrations in the tumor compared to vulnerable side effect organs. Thus, a truly individualized drug dosing therapy for the patient is achieved.

In an embodiment and as preferably used herein, the theragnostic method is realized with only one theragnostically active compound such as a compound of the present invention labeled with a radionuclide emitting diagnostically detectable radiation (e.g. positrons or gamma rays) as well as therapeutically effective radiation (e.g. electrons or alpha particles).

The invention also contemplates a method of intraoperatively identifying/disclosing diseased tissues expressing CAIX in a subject. Such method uses a compound of the invention, whereby such compound of the invention preferably comprises as effector a diagnostically active agent.

According to a further embodiment of the invention, the compound of the invention, particularly if complexed with a radionuclide, may be employed as adjunct or adjuvant to any other tumor treatment including, surgery as the primary method of treatment of most isolated solid cancers, radiation therapy involving the use of ionizing radiation in an attempt to either cure or improve the symptoms of cancer using either sealed internal sources in the form of brachytherapy or external sources, chemotherapy such as alkylating agents, antimetabolites, anthracyclines, plant alkaloids, topoisomerase inhibitors, and other antitumor agents, hormone treatments that modulate tumor cell behavior without directly attacking those cells, targeted agents which directly target a molecular abnormality in certain types of cancer including monoclonal antibodies and tyrosine kinase inhibitors, angiogenesis inhibitors, immunotherapy, cancer vaccination, palliative care including actions to reduce the physical, emotional, spiritual, and psycho-social distress to improve the patient's quality of life and alternative treatments including a diverse group of health care systems, practices, and products that are not part of conventional medicine.

In an embodiment of the methods of the invention, the subject is a patient. In an embodiment, a patient is a subject which has been diagnosed as suffering from or which is suspected of suffering from or which is at risk of suffering from or developing a disease, whereby the disease is a disease as described herein and preferably a disease involving CAIX.

Dosages employed in practicing the methods for treatment and diagnosis, respectively, where a radionuclide is used and more specifically attached to or part of the compound of the invention will vary depending, e.g., on the particular condition to be treated, for example the known radiosensitivity of the tumor type, the volume of the tumor and the therapy desired. In general, the dose is calculated on the basis of radioactivity distribution to each organ and on observed target uptake. A 7-emitting complex may be administered once or at several times for diagnostic imaging. In animals, an indicated dose range may be from 0.1 ng/kg to 5 mg/kg of the compound of the invention complexed, e.g., with 1 kBq to 200 MBq of a 7-emitting radionuclide, including, but not limited to, ¹¹¹In or ⁸⁹Zr. For instance, an indicated dose range of the compound of the invention when complexed with a 7-emitting radionuclide may be from 0.2 mg/kg to 2 mg/kg, e.g., from 0.4 mg/kg to 1 mg/kg, such as about 0.6 mg/kg or 0.8 mg/kg. In an embodiment, an indicated dose range of the compound of the invention when complexed with a 7-emitting radionuclide is from 0.1 μg/kg to 10.0 μg/kg, e.g., 0.1 μg/kg to 5.0 μg/kg, e.g., 0.1 μg/kg to 2.0 μg/kg such as about 0.5 μg/kg, or 0.8 μg/kg, or 1.0 μg/kg. An α- or β-emitting complex of the compound of the invention may be administered at several time points e.g., 1 dose about every 28 days, e.g., over a period of 1 to 3 weeks or longer e.g., over a period of 16 to 32 weeks. In animals, an indicated dosage range may be of from 0.1 ng/kg to 5 mg/kg of the compound of the invention complexed, e.g., with 1 kBq to 200 MBq of an α- or β-emitting radionuclide, including, but not limited to, ²²⁵Ac or ¹⁷⁷Lu. For instance, an indicated dose range of the compound of the invention when complexed with an α- or β-emitting radionuclide may be from 0.2 mg/kg to 2 mg/kg, e.g., from 0.4 mg/kg to 1 mg/kg, such as about 0.6 mg/kg or 0.8 mg/kg. In larger mammals, for example humans, an indicated dosage range is from 0.1 to 100 μg/kg, e.g., 0.1 μg/kg to 10.0 μg/kg, e.g., 0.1 μg/kg to 5.0 μg/kg, e.g., such as about 1.0 μg/kg, or 2.0 μg/kg, or 4.0 μg/kg, of the compound of the invention complexed with, e.g., 10 to 400 MBq ¹¹¹In or ⁸⁹Zr. In larger mammals, for example humans, an indicated dosage range is of from 0.1 ng/kg to 100 μg/kg of the compound of the invention complexed with, e.g., 1 to 100000 MBq of an α- or β-emitting radionuclide, including, but not limited to, ²²⁵Ac or ¹⁷⁷Lu. Preferably, in larger mammals, for example humans, an indicated dosage range of the compound of the invention when complexed with a β-emitting radionuclide such as ¹⁷⁷Lu, e.g., with 200 to 50000 MBq, preferably 500 to 20000 MBq, more preferably 1000 to 15000 MBq, such as 1500 to 10000 MBq of β-emitting radionuclide, is from 0.01 μg/kg to 80 μg/kg, more preferably from 0.1 μg/kg to 50 μg/kg, such as about 1.0 μg/kg to 35 μg/kg, or 2.0 μg/kg to 20 μg/kg. In one aspect, in larger mammals, for example humans, the effective dose resulting from, e.g., the intravenous administration of the compound of the invention complexed with, e.g., 1 to 100000 MBq of an α- or β-emitting radionuclide, including, but not limited to, ²²⁵Ac or ¹⁷⁷Lu, is from 0.01 mSv/MBq to 10.0 mSv/MBq, e.g., 0.1 mSv/MBq to 1 mSv/MBq, such as about 0.1 mSv/MBq to 0.5 mSv/MBq, or 0.2 mSv/MBq to 0.3 mSv/MBq. In one further aspect, in larger mammals, for example humans, the effective dose resulting from, e.g., the intravenous administration of the compound of the invention complexed with a β-emitting radionuclide such as ¹⁷⁷Lu is typically less than 5.0 mSv/MBq, more typically less than 2.0 mSv/MBq, even more typically less than 1.0 mSv/MBq, and most typically less than 0.5 mSv/MBq. Furthermore, in this aspect, the effective dose may be 0.05 mSv/MBq or more, e.g., 0.08 mSv/MBq or more, e.g., 0.1 mSv/MBq or more. In an embodiment, the effective dose resulting from, e.g., the intravenous administration of the compound of the invention complexed with a β-emitting radionuclide such as ¹⁷⁷Lu is less than 1.0 mSv/MBq, e.g., less than 0.5 mSv/MBq, e.g., 0.35 mSv/MBq, such as about 0.25 mSv/MBq. Furthermore, in this embodiment, the effective dose may be 0.1 mSv/MBq or more, e.g., 0.1 mSv/MBq or more, e.g., 0.15 mSv/MBq or more. In another aspect, in larger mammals, for example humans, the radiation dose delivered to a tumor after, e.g., the intravenous administration of the compound of the invention complexed with a β-emitting radionuclide such as m ¹⁷⁷Lu results ranges from about 4.4 to about 660Gy.

### 5. COMPOSITION, PHARMACEUTICAL COMPOSITION AND KIT

In a further aspect, the instant invention is related to a composition and a pharmaceutical composition in particular, comprising the compound of the invention.

The pharmaceutical composition of the present invention comprises at least one compound of the invention and, optionally, one or more carrier substances, excipients and/or adjuvants. The pharmaceutical composition may additionally comprise, for example, one or more of water, buffers such as, e.g., neutral buffered saline or phosphate buffered saline, ethanol, mineral oil, vegetable oil, dimethylsulfoxide, carbohydrates such as e.g., glucose, mannose, sucrose or dextrans, mannitol, proteins, adjuvants, polypeptides or amino acids such as glycine, antioxidants, chelating agents such as EDTA or glutathione and/or preservatives. Furthermore, one or more other active ingredients may, but need not, be included in the pharmaceutical composition of the invention.

The pharmaceutical composition of the invention may be formulated for any appropriate route of administration, including, for example, topical such as, e.g., transdermal or ocular, oral, buccal, nasal, vaginal, rectal or parenteral administration. The term parenteral as used herein includes subcutaneous, intradermal, intravascular such as, e.g., intravenous, intramuscular, intrathecal and intraperitoneal injection, as well as any similar injection or infusion technique. A preferred route of administration is intravenous administration.

In an embodiment of the invention the compound of the invention comprising a radionuclide is administered by any conventional route, in particular intravenously, e.g. in the form of injectable solutions or suspensions. The compound of the invention may also be administered advantageously by infusion, e.g., by an infusion of 30 to 60 min.

Depending on the site of the tumor, the compound of the invention may be administered as close as possible to the tumor site, e.g. by means of a catheter. Such administration may be carried out directly into the tumor tissue or into the surrounding tissue or into the afferent blood vessels. The compound of the invention may also be administered repeatedly in doses, preferably in divided doses.

According to a preferred embodiment of the invention, a pharmaceutical composition of the invention comprises a stabilizer, e.g., a free radical scavenger, which inhibits autoradiolysis of the compound of the invention. Suitable stabilizers include, e.g., serum albumin, ascorbic acid, retinol, gentisic acid or a derivative thereof, or an amino acid infusion solution such, e.g., used for parenteral protein feeding, preferably free from electrolyte and glucose, for example a commercially available amino acid infusion such as Proteinsteril® KE Nephro. Ascorbic acid and gentisic acid are preferred.

A pharmaceutical composition of the invention may comprise further additives, e.g. an agent to adjust the pH between 7.2 and 7.4, e.g. sodium or ammonium acetate or Na₂HPO₄. Preferably, the stabilizer is added to the non-radioactive compound of the invention and introduction of the radionuclide, for instance the complexation with the radionuclide, is performed in the presence of the stabilizer, either at room temperature or, preferably, at a temperature of from 40 to 120° C. The complexation may conveniently be performed under air free conditions, e.g., under N₂ or Ar. Further stabilizer may be added to the composition after complexation.

Excretion of the compound of the invention, particularly if the effector comprises a radionuclide, essentially takes place through the kidneys. Further protection of the kidneys from radioactivity accumulation may be achieved by administration of lysine or arginine or an amino acid solution having a high content of lysine and/or arginine, e.g., a commercially available amino acid solution such as Synthamin®-14 or -10, prior to the injection of or together with the compound of the invention, particularly if the effector is a radionuclide. Protection of the kidneys may also be achieved by administration of plasma expanders such as, e.g., gelofusine, either instead of or in addition to amino acid infusion. Protection of the kidneys may also be achieved by administration of diuretics providing a means of forced diuresis which elevates the rate of urination. Such diuretics include high ceiling loop diuretics, thiazides, carbonic anhydrase inhibitors, potassium-sparing diuretics, calcium-sparing diuretics, osmotic diuretics and low ceiling diuretics. A pharmaceutical composition of the invention may contain, apart from a compound of the invention, at least one of these further compounds intended for or suitable for kidney protection, preferably kidney protection of the subject to which the compound of the invention is administered.

It will be understood by a person skilled in the art that the compound of the invention is disclosed herein for use in various methods. It will be further understood by a person skilled in the art that the composition of the invention and the pharmaceutical composition of the invention can be equally used in said various methods. It will also be understood by a person skilled in the art that the composition of the invention and the pharmaceutical composition are disclosed herein for use in various methods. It will be equally understood by a person skilled in the art that the compound of the invention can be equally used in said various methods.

It will be acknowledged by a person skilled in the art that the composition of the invention and the pharmaceutical composition of the invention contain one or more further compounds in addition to the compound of the invention. To the extent that such one or more further compounds are disclosed herein as being part of the composition of the invention and/or of the pharmaceutical composition of the invention, it will be understood that such one or more further compounds can be administered separately from the compound of the invention to the subject which is exposed to or the subject of a method of the invention. Such administration of the one or more further compounds can be performed prior, concurrently with or after the administration of the compound of the invention. It will also be acknowledged by a person skilled in the art that in a method of the invention, apart from a compound of the invention, one or more further compound may be administered to a subject. Such administration of the one or more further compounds can be performed prior, concurrently with or after the administration of the compound of the invention. To the extent that such one or more further compounds are disclosed herein as being administered as part of a method of the invention, it will be understood that such one or more further compounds are part of a composition of the invention and/or of a pharmaceutical composition of the invention. It is within the present invention that the compound of the invention and the one or more further compounds may be contained in the same or a different formulation. It is also within the present invention that the compound of the invention and the one or more further compounds are not contained in the same formulation, but are contained in the same package containing a first formulation comprising a compound of the invention, and a second formulation comprising the one or more further compounds, whereby the type of formulation may be the same or may be different.

It is within the present invention that more than one type of a compound of the invention is contained in the composition of the invention and/or the pharmaceutical composition of the invention. It is also within the present invention that more than one type of a compound of the invention is used, preferably administered, in a method of the invention.

It will be acknowledged that a composition of the invention and a pharmaceutical composition of the invention may be manufactured in conventional manner.

Radiopharmaceuticals have decreasing content of radioactivity with time, as a consequence of the radioactive decay. The physical half-life of the radionuclide is often short for radiopharmaceutical diagnostics. In these cases, the final preparation has to be done shortly before administration to the patient. This is in particular the case for positron emitting radiopharmaceuticals for tomography (PET radiopharmaceuticals). It often leads to the use of semi-manufactured products such as radionuclide generators, radioactive precursors and kits.

Preferably, a kit of the invention comprises apart from one or more than one compounds of the invention typically at least one of the followings: instructions for use, final preparation and/or quality control, one or more optional excipient(s), one or more optional reagents for the labeling procedure, optionally one or more radionuclide(s) with or without shielded containers, and optionally one or more device(s), whereby the device(s) is/are selected from the group comprising a labeling device, a purification device, an analytical device, a handling device, a radioprotection device or an administration device.

Shielded containers known as “pigs” for general handling and transport of radiopharmaceutical containers come in various configurations for holding radiopharmaceutical containers such as bottles, vials, syringes, etc. One form often includes a removable cover that allows access to the held radiopharmaceutical container. When the pig cover is in place, the radiation exposure is acceptable.

A labeling device is selected from the group of open reactors, closed reactors, microfluidic systems, nanoreactors, cartridges, pressure vessels, vials, temperature controllable reactors, mixing or shaking reactors and combinations thereof.

A purification device is preferably selected from the group of ion exchange chromatography columns or devices, size-exclusion chromatography columns or devices, affinity chromatography columns or devices, gas or liquid chromatography columns or devices, solid phase extraction columns or devices, filtering devices, centrifugations vials columns or devices.

An analytical device is preferably selected from the group of tests or test devices to determine the identity, radiochemical purity, radionuclidic purity, content of radioactivity and specific radioactivity of the radiolabelled compound.

A handling device is preferably selected from the group consisting of devices for mixing, diluting, dispensing, labeling, injecting and administering radiopharmaceuticals to a subject.

A radioprotection device is used in order to protect doctors and other personnel from radiation when using therapeutic or diagnostic radionuclides. The radioprotection device is preferably selected from the group consisting of devices with protective barriers of radiation-absorbing material selected from the group consisting of aluminum, plastics, wood, lead, iron, lead glass, water, rubber, plastic, cloth, devices ensuring adequate distances from the radiation sources, devices reducing exposure time to the radionuclide, devices restricting inhalation, ingestion, or other modes of entry of radioactive material into the body and devices providing combinations of these measures.

An administration device is preferably selected from the group of syringes, shielded syringes, needles, pumps, and infusion devices. Syringe shields are commonly hollow cylindrical structures that accommodate the cylindrical body of the syringe and are constructed of lead or tungsten with a lead glass window that allows the handler to view the syringe plunger and liquid volume within the syringe.

It is within the present invention that the term “in an embodiment” or “in an embodiment of the invention” refers to each and any aspect of the invention, including any embodiment thereof.

### 6. FIGURES

The present invention is now further illustrated by reference to the following figures and examples from which further features, embodiments and advantages, may be taken, wherein

FIG. 1 shows the amino acid sequences of human carbonic anhydrase 9 (CAIX) (SEQ ID NO: 4), human carbonic anhydrase 4 (CAIV) (SEQ ID NO: 5), human carbonic anhydrase 12 (CAXII) (SEQ ID NO: 6), human carbonic anhydrase 14 (CAXIV) (SEQ ID NO: 7), canine carbonic anhydrase 9 (CAIX) (SEQ ID NO: 8), and murine carbonic anhydrase 9 (CAIX) (SEQ ID NO:9).

FIG. 2 shows a radiochromatogram of ¹¹¹In-3BP-3478 (A) and ¹¹¹In-3BP-3583 (B), with all peaks with an HPLC area ≥0.5% labeled with their retention times.

FIG. 3 shows a radiochromatogram of ¹¹¹In-3BP-3840 (A) and ¹¹¹In-3BP-4175 (B), with all peaks with an HPLC area ≥0.5% labeled with their retention times.

FIG. 4 shows a radiochromatogram of ¹¹¹In-3BP-4237 (A) and ¹¹¹In-3BP-4452 (B), with all peaks with an HPLC area ≥0.5% labeled with their retention times.

FIG. 5 shows a radiochromatogram of ¹¹¹In-3BP-4501 (A) and ¹¹¹In-3BP-4503 (B), with all peaks with an HPLC area ≥0.5% labeled with their retention times.

FIG. 6 shows the percentage of injected dose per gram of tissue (% ID/g) uptake in the kidneys, liver, blood pool and SK-RC-52 tumor as determined by SPECT-imaging of ¹¹¹In-3BP-3478 (A) and ¹¹¹In-3BP-3583 (B) 1 h, 3 h, 6 h and 24 h post injection into the mouse model.

FIG. 7 shows the percentage of injected dose per gram of tissue (% ID/g) uptake in the kidneys, liver, blood pool and indicated tumors as determined by SPECT-imaging of ¹¹¹In-3BP-3840 (A) and ¹¹¹In-3BP-4175 (B) 1 h, 3 h, 6 h and 24 h post injection into the mouse model.

FIG. 8 shows the percentage of injected dose per gram of tissue (% ID/g) uptake in the kidneys, liver, blood pool and SK-RC-52 tumor as determined by SPECT-imaging of ¹¹¹In-3BP-4237 1 h, 4 h, 6 h and 24 h post injection into the mouse model.

FIG. 9 shows the percentage of injected dose per gram of tissue (% ID/g) uptake in the kidneys, liver, blood pool, SK-RC-52 tumor and HT-29 tumor as determined by SPECT-imaging of ¹¹¹In-3BP-4369 (A) and ¹¹¹In-3BP-4400 (B) 1 h, 3 h, 6 h and 24 h post injection into the mouse model.

FIG. 10 shows the percentage of injected dose per gram of tissue (% ID/g) uptake in the kidneys, liver, blood pool, SK-RC-52 tumor and HT-29 tumor as determined by SPECT-imaging of ¹¹¹In-3BP-4448 1 h, 4 h, and 24 h (A) and ¹¹¹In-3BP-4452 1 h, 4 h, 24 and 48 h (B) post injection into the mouse model.

FIG. 11 shows the percentage of injected dose per gram of tissue (% ID/g) uptake in the kidneys, liver, blood pool, SK-RC-52 tumor and HT-29 tumor as determined by SPECT-imaging of ¹¹¹In-3BP-4453 (A) and ¹¹¹In-3BP-4455 (B) 1 h, 4 h, and 24 h post injection into the mouse model.

FIG. 12 shows the percentage of injected dose per gram of tissue (% ID/g) uptake in the kidneys, liver, blood pool, SK-RC-52 tumor and HT-29 tumor as determined by SPECT-imaging of ¹¹¹In-3BP-4501 (A) and ¹¹¹In-3BP-4503 (B) 1 h, 4 h, 24, and 48 h post injection into the mouse model.

FIG. 13 shows the percentage of injected dose per gram of tissue (% ID/g) uptake in the kidneys, liver, blood pool, SK-RC-52 tumor and HT-29 tumor as determined by SPECT-imaging of ¹¹¹In-3BP-4504 (A) and ¹¹¹In-3BP-4505 (B) 1 h, 4 h, and 24 h post injection into the mouse model.

FIG. 14 shows SPECT-images of ¹¹¹In-3BP-3478 or ¹¹¹In-3BP-3583 3 h post injection into mice bearing SK-RC-52 tumors (A), of ¹¹¹In-3BP-4175 3 h post injection post injection into mice bearing SK-RC-52 and HT-29 tumors (B), of ¹¹¹In-3BP-4452 or ¹¹¹In-3BP-4501 or ¹¹¹In-3BP-4503 4 h post injection into mice bearing SK-RC-52 and HT-29 tumors (C). SK-RC-52 tumors are located on the right shoulder and HT-29 tumors on the left shoulder.

FIG. 15 shows the in vivo efficacy in terms of tumor volume (A), impact on relative body weight (B) and tumor uptake (C) of ¹⁷⁷Lu-DPI-4452 in the HT-29 xenograft mouse model.

FIG. 16 shows the in vivo uptake of ¹⁷⁷Lu-DPI-4452 in kidneys (A) and liver (B) as well as the comparison of the uptake of ¹⁷⁷Lu-DPI-4452 and ⁶⁸Ga-DPI-4452 in kidney, liver and tumor (C) in the HT-29 xenograft mouse model.

FIG. 17 shows the in vivo images of intravenously injected ⁶⁸Ga-DPI-4452 at 1 hour p.i. and ¹⁷⁷Lu-DPI-4452 at 4 hours p.i. in the HT29 xenograft mouse model. Representative axial, coronal and maximal intensity (bottom) projection (MIP) images of two mice are shown (FIG. 17A: first mouse, FIG. 17B: second mouse). Uptake is presented as percent injected dose per gram tissue (% ID/g).

FIG. 18 shows the in vivo efficacy in terms of tumor volume (A), impact on relative body weight (B) and tumor uptake (C) of ¹⁷⁷Lu-DPI-4452 in the SK-RC-52 xenograft mouse model.

FIG. 19 shows the in vivo uptake of ¹⁷⁷Lu-DPI-4452 in kidneys (A) and liver (B) as well as the comparison of the uptake of ¹⁷⁷Lu-DPI-4452 and ⁶⁸Ga-DPI-4452 in kidney, liver and tumor (C) in the SK-RC-52 xenograft mouse model.

FIG. 20 shows the in vivo imaging of ¹⁷⁷Lu-DPI-4452 in the SK-RC-52 xenograft mouse model. Representative axial, coronal and maximal intensity (bottom) projection (MIP) images for two mice are shown. Uptake is presented as percent injected dose per gram tissue (% ID/g).

FIG. 21 shows inclusion based on SK-RC-52 tumor volume and body weight at the day of dosing. No significant difference in tumor volume (p=0.80, one-way ANOVA) or body weight (p=0.96, one-way ANOVA) was observed between the groups. N=3/group, Mean SEM.

FIG. 22 shows inclusion based on HT-29 tumor volume and body weight at day −1 (the day before dosing). No significant difference in tumor volume (p=0.80, unpaired t-test) or body weight (p=0.32, unpaired t-test) was observed between the groups. N=3/group, Mean SEM.

FIG. 23 shows representative SPECT/CT images (axial, coronal and maximum intensity projection images) of one mouse from group A1 at 1, 4, 24 and 48 hours post injection of [¹¹¹In]In-DPI-4452.

FIG. 24 shows representative SPECT/CT images (axial, coronal and maximum intensity projection images) of one mouse from group A2 at 1, 4, 24 and 48 hours post injection of [¹¹¹In]In-DPI-4501.

FIG. 25 shows representative SPECT/CT images (axial, coronal and maximum intensity projection images) of one mouse from group A3 at 1, 4, 24 and 48 hours post injection of [¹¹¹In]In-DPI-4452+gelofusine.

FIG. 26 shows representative SPECT/CT images (axial, coronal and maximum intensity projection images) of one mouse from group A4 at 1, 4, 24 and 48 hours post injection of [¹¹¹In]In-DPI-4501+gelofusine.

FIG. 27 shows representative SPECT/CT images (axial, coronal and maximum intensity projection images) of one mouse from group Bi at 2, 4, 24 and 48 hours post injection of [¹¹¹In]In-DPI-4452.

FIG. 28 shows representative SPECT/CT images (axial, coronal and maximum intensity projections images) of one mouse from group B2 at 2, 4, 24 and 48 hours post injection of [¹¹¹In]In-DPI-4501.

FIG. 29 shows percentage of injected dose per gram of tissue (% ID/g) uptake of [¹¹¹In]In-DPI-4452 and [¹¹¹In]In-DPI-4501 in SK-RC-52 and HT-29 tumor, kidneys, liver and blood. Uptake in the SK-RC-52 tumor mouse model was compared with injection of gelofusine immediately before injection of compounds. N=3/group, Mean±SEM.

FIG. 30 shows percentage of injected dose per gram of tissue (% ID/g) uptake of [¹¹¹In]In-DPI-4452 and [¹¹¹In]In-DPI-4501 in SK-RC-52 and HT-29 tumor, kidneys, liver and blood (logarithmic scale). Uptake in the SK-RC-52 tumor model was compared with injection of gelofusine immediately before injection of compounds. N=3/group, Mean±SEM.

FIG. 31 shows [¹¹¹In]In-DPI-4452 versus [¹¹¹In]In-DPI-4501 pharmacokinetics in dog blood (% ID/g). Activity measurement in ex vivo blood samples from males (left) and females (right) after injection with In-111-labeled DPI-4452 and In-111-labeled DPI-4501. Y-axis presented in log 10. N=2/group. Plots represent mean±SEM.

FIG. 32 shows male versus female dog pharmacokinetics in blood (% ID/g). Activity measurement in ex vivo blood samples from males and females after injection with In-111-labeled DPI-4452 (left) and In-111-labeled DPI-4501 (right), respectively. Y-axis presented in log 10. N=2/group. Plots represent mean±SEM.

FIG. 33 shows [¹¹¹In]In-DPI-4452 versus [¹¹¹In]In-DPI-4501 pharmacokinetics in dog urine (% ID/g). Activity measurement in ex vivo urine samples from males (left) and females (right) after injection with In-111-labeled DPI-4452 and In-111-labeled DPI-4501. Y-axis presented in log 10. N=2/group. Plots represent mean±SEM.

FIG. 34 shows male versus female dog pharmacokinetics in urine (% ID/g). Activity measurement in ex vivo urine samples from males and females after injection with respectively In-111-labeled DPI-4452 (left) and In-111-labeled DPI-4501 (right). Y-axis presented in log 10. N=2/group. Plots represent mean±SEM.

FIG. 35 shows SPECT/CT-derived biodistribution data of [¹¹¹In]In-DPI-4452 (% ID/g and SUV) in male and female dogs. Graphs represent imaging time points of 1 h (left), 4 h (middle), and 48 h (right) post injection, respectively. X-axis present the investigated organs.

N=2/group (N=1 in female 4 h scan group). Plots represent mean±SEM.

FIG. 36 shows SPECT/CT-derived biodistribution data of [¹¹¹In]In-DPI-4501 (% ID/g and SUV) in male and female dogs. Graphs represent imaging time points of 1 h (left), 4 h (middle), and 48 h (right) post injection, respectively. X-axis present the investigated organs.

N=2/group. Plots represent mean±SEM.

FIG. 37 shows representative SPECT/CT images of [¹¹¹In]In-DPI-4452 biodistribution in female dogs. Scan images of one female beagle dog at respectively 1 hour, 4 hours and 48 hours after injection. Scalebar represents SUV values.

FIG. 38 shows representative SPECT/CT images of [¹¹¹In]In-DPI-4452 biodistribution in male dogs. Scan images of one male beagle dog at respectively 1 hour, 4 hours and 48 hours after injection. Scalebar represents SUV values.

FIG. 39 shows representative SPECT/CT images of [¹¹¹In]In-DPI-4501 biodistribution in female dogs. Scan images of one female beagle dog at respectively 1 hour, 4 hours and 48 hours after injection. Scalebar represents SUV values.

FIG. 40 shows representative SPECT/CT images of [¹¹¹In]In-DPI-4501 biodistribution in male dogs. Scan images of one male beagle dog at respectively 1 hour, 4 hours and 48 hours after injection. Scalebar represents SUV values.

FIG. 41 shows the mean total plasma concentration of DPI-4452 versus time profiles following a single i.v. bolus injection of 25, 80, 400 and 800 μg/kg DPI-4452 in male beagle dogs. N=6/group, Mean±SD.

FIG. 42 shows the mean total plasma concentration of 16, 80, and 400 μg/kg DPI-4452 versus time profiles following a single i.v. bolus injection of DPI-4452 in beagle dogs. N=2, Mean±SD.

FIG. 43 shows in vivo hematological analysis results following administration of ¹⁷⁷Lu-DPI-4452 to HT-29-xenografted mice. The X-axis represents the study day post injection. QW indicates the weekly dosing regimen.

FIG. 44 shows in vivo hematological analysis results following administration of ¹⁷⁷Lu-DPI-4452 to SK-RC-52-xenografted mice. The X-axis represents the study day post injection. QW indicates the weekly dosing regimen.

FIG. 45 shows the in vivo creatinine (μmol/L) and urea (mmol(L) levels following administration of ¹⁷⁷Lu-DPI-4452 to SK-RC-52-xenograftedmice. The X-axis represents the study day post injection. QW indicates the weekly dosing regimen.

FIG. 46 shows the in vivo efficacy in terms of tumor volume (A) and impact on relative body weight (B) of a single bolus injection of different doses of ²²⁵Ac-DPI-4452 in the HT-29 xenograft mouse model.

FIG. 47 shows ex vivo biodistribution data (assessed in an automated gamma counter after reaching secular equilibrium) following administration of ²²⁵Ac-DPI-4452 (% ID/g) to HT-29-xenografted mice. Graphs represent the time point of 4 h post injection. The X-axis represents the investigated organs.

FIG. 48 shows in vivo hematological analysis results following administration of ²²⁵Ac-DPI-4452 to HT-29-xenografted mice. The X-axis represents the study day post injection.

FIG. 49 shows the in vivo creatinine (μmol/L) and urea (mmol(L) levels following administration of ²²⁵Ac-DPI-4452 to HT-29 xenograft model mice. The X-axis represents the study day post injection.

FIG. 50 show the in vivo efficacy (A) and impact on body weight (B) of a single bolus injection of different doses of ²²⁵Ac-DPI-4452 in the SK-RC-52 xenograft mouse model.

FIG. 51 shows ex vivo derived biodistribution data (assessed in an automated gamma counter after reaching secular equilibrium) following administration of ²²⁵Ac-DPI-4452 (% ID/g) to SK-RC-52-xenografted mice. Graphs represent the time point of 4 h post injection. The X-axis represents the investigated organs.

FIG. 52 shows in vivo hematological analysis results following administration of ²²⁵Ac-DPI-4452 to SK-RC-52-xenografted mice. The X-axis represents the study day post injection.

FIG. 53 shows the in vivo creatinine (μmol/L) and urea (mmol(L) levels following administration of ²²⁵Ac-DPI-4452 to SK-RC-52-xenografted mice. The X-axis represents the study day post injection.

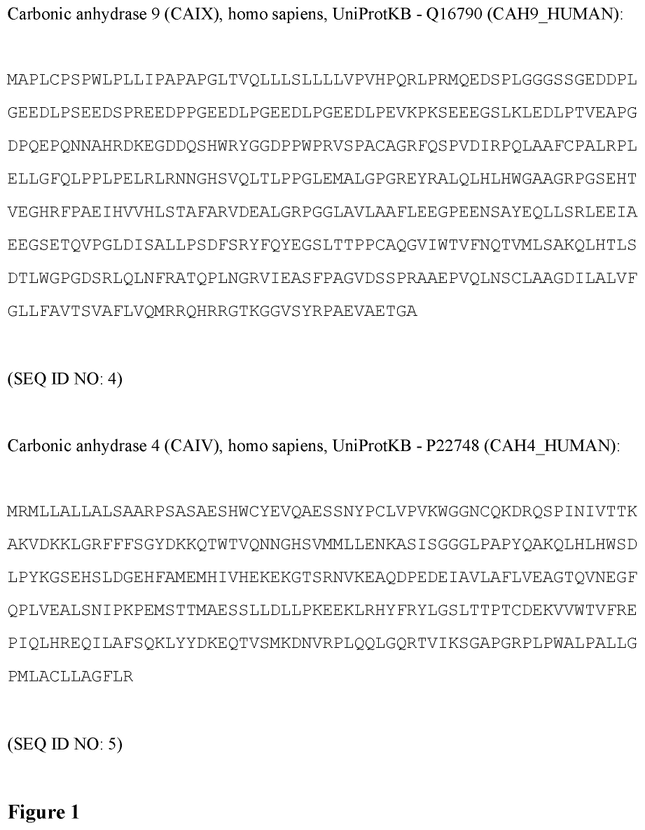

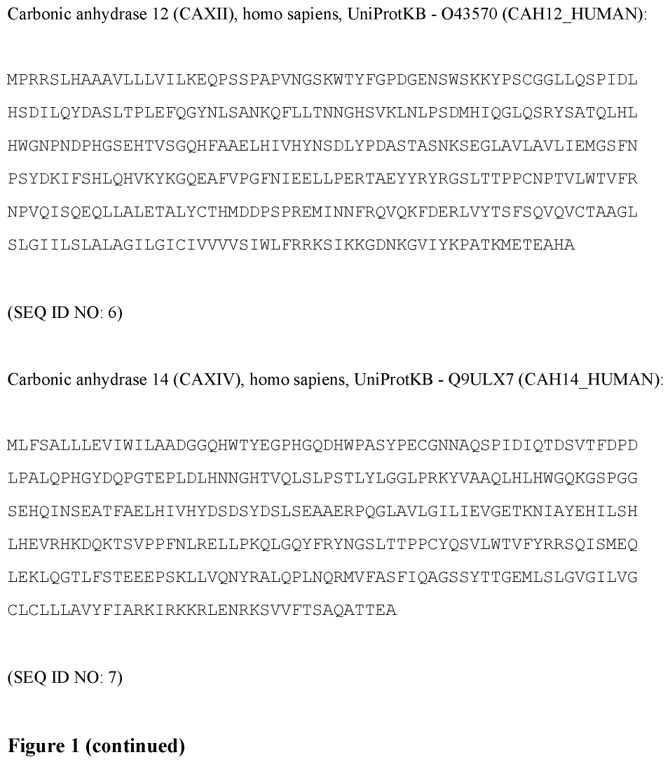

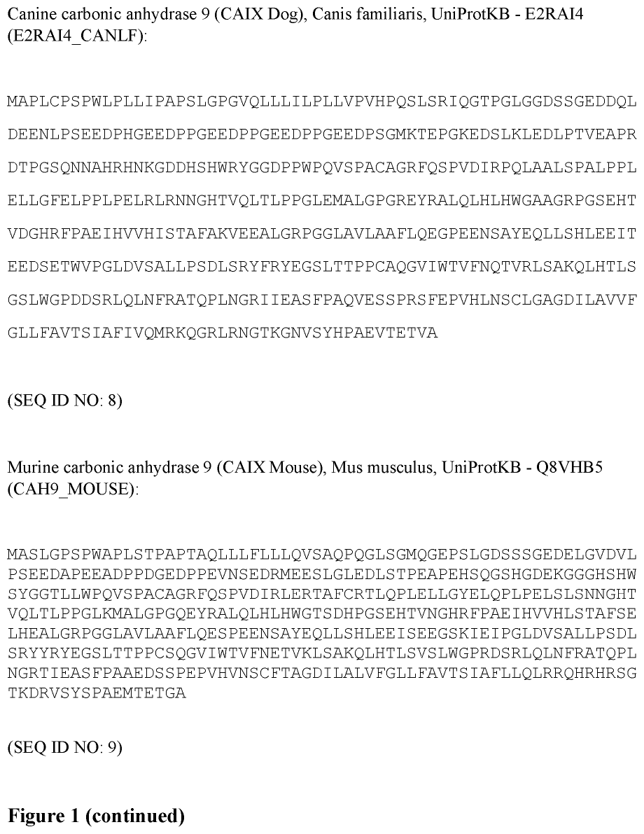

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### 7. EXAMPLES

Abbreviations used in the instant application and the following examples in particular are as follows:

| | | |
|---|---|---|
| 3MeBn | means | m-methylbenzyl |
| ACN | means | acetonitrile |
| ADCC | means | antibody-dependent cell-mediated cytotoxicity |
| ADP | means | adenosine diphosphate |
| Af3 | means | L-3-aminophenylalanine |
| Ahx | means | 6-aminohexanoic acid |
| Alloc | means | allyloxycarbonyl |
| AMC | means | 7-amino-4-methylcoumarin |
| amu | means | atomic mass unit |
| ANOVA | means | analysis of variance |
| APAc | means | 2-(4-(amino)piperidin-1-yl)acetic acid |
| APC | means | allophycocyanin |
| Ape | means | 5-aminopentanol |
| Aph | means | 4-aminophenylalanine |
| Apr | means | 3-aminopropanol |
| aq. | means | aqueous |
| AUC | means | area under the curve |
| AUC_{tLast} | means | area under the curve up to the last measured time point |
| BLAST | means | basic local alignment search tool |
| BLQ | means | below limit of quantification |
| BSA | means | bovine serum albumin |
| CA | means | carbonic anhydrase |
| CAF | means | cancer-associated fibroblast |
| CAI | means | carbonic anhydrase I |
| CAII | means | carbonic anhydrase II |
| CAIII | means | carbonic anhydrase III |
| CAIV | means | carbonic anhydrase IV |
| CAIX | means | carbonic anhydrase IX |
| calc | means | calculated |
| CARP | means | carbonic anhydrase-related proteins |
| CAR-T | means | chimeric antigen receptor T |
| CAVA | means | carbonic anhydrase V a |
| CAVB | means | carbonic anhydrase V b |
| CAVI | means | carbonic anhydrase VI |
| CAVII | means | carbonic anhydrase VII |
| CAVIII | means | carbonic anhydrase VIII |
| CAX | means | carbonic anhydrase X |
| CAXII | means | carbonic anhydrase XII |
| CAXIII | means | carbonic anhydrase XIII |
| CAXIV | means | carbonic anhydrase XIV |
| ccRCC | means | clear cell renal cell carcinoma |
| CGH | means | comparative genomic hybridization |
| CHO | means | chinese hamster ovary cell line |
| CL | means | clearance |
| CM | means | ChemMatrixTM |
| CNP | means | C-type natriuretic peptide |
| CNS | means | central nervous system |
| Cpsu | means | 3-Carboxypropanesulfonamide |
| CRC | means | colorectal cancer |
| CT | means | computed tomography |
| Cy5 | means | cyanine-5 |
| DAD | means | diode array detector |
| Dap | means | diamino propionic acid |
| DCM | means | dichloromethane |
| Dde | means | N-(1-(4,4-dimethyl-2,6-dioxocyclohexylidene)ethyl) |
| DEG | means | diethylene glycol dimethacrylate |
| DEPBT | means | (3-(diethoxyphosphoryloxy)-1,2,3-benzotriazin-4(3H)-one) |
| DIC | means | N,N′-diisopropylcarbodiimide |
| DICOM | means | digital imaging and communications in medicine |
| DIPEA | means | diisopropylethylamine |
| DMF | means | N,N-dimethylformamide |
| DMSO | means | dimethyl sulfoxide |
| DNA | means | deoxyribonucleic acid |
| DOTA | means | 1,4,7,10-tetraazacyclododecane-1,4,7,10-tetraacetic acid |
| DOTA(tBu)3-OH | means | tri-tert-butyl-1,4,7,10-tetraazacyclo-dodecane-1,4,7,10- |
| | | tetraacetate |
| dPBS | means | Dulbecco's phosphate-buffered saline |
| DTPA | means | diethylenetriamine pentaacetate |
| e.g. | means | for example (exempli gratia) |
| EC | means | electron capture |
| EC₅₀ | means | half-maximal excitatory concentration |
| ECACC | means | European Collection of Authenticated Cell Cultures |
| ECG | means | electrocardiogram |
| EDC | means | 1-ethyl-3-(3-dimethylaminopropyl)carbodiimide |
| EDT | means | 1,2-ethanedithiol |
| EDTA | means | ethylenediaminetetraacetic acid |
| ELISA | means | enzyme-linked immunosorbent assay |
| EMEM | means | Eagle's minimum essential medium |
| eq. | means | equivalent |
| ESI | means | electrospray ionization |
| Et₂O | means | diethylether |
| EtOAc | means | ethylacetate |
| EtOH | means | ethanol |
| FACS | means | fluorescence-activated cell sorting |
| Fc | means | fragment crystallizable region (of an antibody) |
| FCS | means | fetal calf serum |
| FFPE | means | formalin-fixed paraffin-embedded |
| FITC | means | 5(6)-fluorescein isothiocyanate |
| Fmoc | means | 9-fluorenylmethoxycarbonyl |
| FOB | means | functional observational battery |
| FRET | means | Fluorescence Resonance Energy Transfer |
| Gab | means | gamma-amino butyric acid |
| GABA | means | gamma-amino butyric acid |
| GBq | means | gigabecquerel |
| GLP | means | good laboratory practice |
| GMP | means | good manufacturing practices |
| h | means | hour(s) |
| HATU | means | O-(7-azabenzotriazol-1-yl)-N,N,N′,N′-tetramethyluronium |
| | | hexafluorophosphate |
| hCAIX | means | human carbonic anhydrase IX |
| HEPES | means | 4-(2-hydroxyethyl)-1-piperazineethanesulfonic acid |
| HFIP | means | hexafluoro-2-isopanol |
| HIF-1α | means | hypoxia-inducible factor 1α |
| HOAc | means | acetic acid |
| HOAt | means | 1-hydroxy-7-azabenzotriazole |
| HPLC | means | high performance liquid chromatography |
| i.e. | means | that is (id est) |
| IC50 | means | half-maximal inhibitory concentration |
| ICRP | means | International Commission on Radiation Protection |
| ID | means | injected dose |
| ID/g | means | injected dose per gram |
| IDBS | means | ID Business Solutions |
| IHC | means | immunohistochemistry |
| i.m. | means | intramuscularly |
| IS | means | isomeric transition |
| IT | means | isomeric transition |
| i.v. | means | intravenous |
| IUPAC | means | International Union of Pure and Applied Chemistry |
| KD | means | dissociation constant |
| kDa | means | kilo Dalton |
| Kᵢ | means | inhibitory constant |
| k_{off} | means | dissociation rate |
| kₒₙ | means | association rate |
| kVp | means | kilovoltage peak |
| LC-HRMS | means | liquid chromatography coupled with high resolution mass |
| | | spectrometry |
| LC-MS | means | high performance liquid chromatography coupled with mass |
| | | spectrometry |
| LC/TOF-MS | means | liquid chromatography time-of-flight mass spectrometry |
| LDH | means | lactate dehydrogenase |
| LiOH | means | lithium hydroxide |
| M | means | molar or mol per Liter |
| m/z | means | mass divided by charge |
| mAb | means | monoclonal antibody |
| max. | means | maximum |
| MBq | means | megabecquerel |
| MeOH | means | methanol |
| MeV | means | mega electron volt |
| min | means | minute(s) |
| MIP | means | maximum intensity projection |
| MIRD | means | Medical Internal Radiation Dose |
| MLPA | means | multiplex ligation-dependent probe amplification |
| MMAE | means | monomethylauristatin E |
| MMP | means | matrix metalloproteinase |
| mRNA | means | messenger ribonucleic acid |
| MS | means | mass spectrometry |
| MTBE | means | methyl-tert-butylether |
| Mtt | means | methyltrityl |
| MW | means | molecular weight |
| n.a. | means | not applicable |
| n.d | means | not determined |
| Na2SO4 | means | sodium sulfate |
| NaCl | means | sodium chloride |
| NaHCO3 | means | sodium hydrogencarbonate |
| NCA | means | non-compartmental |
| NCBI | means | National Center for Biotechnology Information |
| NEP | means | neutral endopeptidase |
| NHS | means | N-hydroxysuccinimide |
| Nlys | means | 4-aminobutyl-glycine |
| NMM | means | 4-methylmorpholine |
| NMP | means | 1-methyl-2-pyrrolidone |
| NOAEL | means | no observed adverse effect level |
| NOS | means | not otherwise specified |
| O2Oc | means | 3,6-dioxaoctanoic acid |
| O2PhiPr | means | 2-phenylisopropyl |
| Oic | means | L-octahydroindol-2-carbonsaure |
| OLINDA | means | Organ Level INternal Dose Assessment/EXponential Modeling |
| p.a. | means | for analytical purpose (quality grade) |
| PARP | means | poly ADP ribose polymerase |
| Pbf | means | 2,2,4,6,7-pentamethyl-2,3 -dihydrobenzofuran-5-sulfonyl |
| PBS | means | phosphate buffered saline |
| PBST | means | phosphate buffered saline containing Tween |
| PCR | means | polymerase chain reaction |
| PDAC | means | pancreatic ductal adenocarcinoma |
| PE | means | polyethene |
| pEC₅₀ | means | negative decadic logarithm of EC50 value when converted to |
| | | molar |
| PET | means | positron emission tomography |
| pIC₅₀ | means | negative decadic logarithm of IC50 value when converted to |
| | | molar |
| PK | means | pharmacokinetic |
| pK_{D} | means | negative decadic logarithm of dissociation constant when |
| | | converted to molar |
| POP | means | prolyl oligopeptidase |
| PREP | means | prolyl endopeptidase |
| prep. | means | preparative |
| PS | means | polystyrene |
| QC | means | quality control |
| Q-RT-PCR | means | quantitative real-time polymerase chain reaction |
| Q-TOF | means | quadrupole time-of-flight |
| QW | means | once weekly |
| RCC | means | renal cell carcinoma |
| RCP | means | radiochemical purity |
| RFU | means | relative fluorescence unit |
| RLB | means | radioligand binding assay |
| RNA | means | ribonucleic acid |
| RP | means | reversed phase |
| RRID | means | Research Resource Identifier |
| RT | means | room temperature |
| Rt | means | retention time |
| RU | means | resonance units |
| SaPr | means | 3-sufamoylpropanoic acid |
| sat. | means | saturated |
| SCCNC | means | squamous cell carcinoma of head and neck |
| scFv | means | single-chain variable fragment |
| SCK | means | single cycle kinetic |
| SD | means | standard deviation |
| sec | means | second |
| SEM | means | standard error of means |
| SF | means | spontaneous fission |
| SPECT | means | single photon emission computed tomography |
| SPPS | means | solid phase peptide synthesis |
| SPR | means | surface plasmon resonance |
| Sq. NSCLC | means | squamous non-small cell lung carcinoma |
| tBu | means | tert-butyl |
| TFA | means | trifluoroacetate or trifluoroacetic acid |
| TG | means | TentaGel |
| TIPS | means | triisopropylsilane |
| TK | means | toxicokinetics |
| TLC | means | thin layer chromatography |
| TMA | means | tissue microarray |
| TME | means | tumor microenvironment |
| TNBC | means | triple-negative breast cancer |
| UHPLC | means | ultrahigh performance liquid chromatography |
| UV | means | ultraviolet |
| VGT | means | vertical gene transfer |
| VHL | means | von Hippel-Lindau |
| Vss | means | volume of distribution at steady state |
| WBC | means | white blood cells |

### Example 1: Material and Methods

The materials and methods as well as general methods are further illustrated by the following examples.

### Solvents:

Solvents were used in the specified quality without further purification. Acetonitrile (Super Gradient, HPLC, VWR—for analytical purposes; PrepSolv, Merck—for preparative purposes); dichloromethane (synthesis, Roth); ethyl acetate (synthesis grade, Roth); N,N-dimethylformamide (peptide synthesis grade, Biosolve); 1-methyl-2-pyrolidone (peptide grade, IRIS BioTech) 1,4-dioxane (reinst, Roth); methanol (p. a., Merck).

Water: Milli-Q Plus, Millipore, demineralized.

### Chemicals:

Chemicals were synthesized according to or in analogy to literature procedures or purchased from Sigma-Aldrich-Merck (Deisenhofen, Germany), Bachem (Bubendorf, Switzerland), VWR (Darmstadt, Germany), Novabiochem (Merck Group, Darmstadt, Germany), Acros Organics (distribution company Fisher Scientific GmbH, Schwerte, Germany), Iris Biotech (Marktredwitz, Germany), Amatek Chemical (Jiangsu, China), Roth (Karlsruhe, Deutschland), Molecular Devices (Chicago, USA), Biochrom (Berlin, Germany), Peptech (Cambridge, MA, USA), Synthetech (Albany, OR, USA), Pharmacore (High Point, NC, USA), PCAS Biomatrix Inc (Saint-Jean-sur-Richelieu, Quebec, Canada), Alfa Aesar (Karlsruhe, Germany), Tianjin Nankai Hecheng S&T Co., Ltd (Tianjin, China), CheMatech (Dijon, France) and Anaspec (San Jose, CA, USA) or other companies and used in the assigned quality without further purification.

### HPLC/MS Analyses:

HPLC/MS analyses were performed by injection of 5 μl of a solution of the sample, using a 2-step gradient for all chromatograms (5-65% B in 12 min, followed by 65-90% in 0.5 min, A: 0.1% TFA in water and B: 0.1% TFA in ACN). RP columns were from Agilent (Type Poroshell 120, 2.7 μm, EC-C18, 50×3.00 mm, flow 0.8 ml, HPLC at room temperature); Mass spectrometer: Agilent 6230 LC/TOF-MS, ESI ionization. MassHunter Qualitative Analysis B.07.00 SP2 was used as software. UV detection was done at k=230 nm. Retention times (Rₜ) are indicated in the decimal system (e.g., 1.9 min=1 min 54 s) and are referring to detection in the UV spectrometer. For the evaluation of observed compound masses the ‘Find Compounds by Formula’-feature was used. In particular, the individual ‘neutral mass of a compound (in units of Daltons)’-values and the corresponding isotope distribution pattern were used to confirm compound identity. The accuracy of the mass spectrometer was approx.±5 ppm.

### Preparative HPLC:

Preparative HPLC separations were done with reversed phase columns (Kinetex 5μ XB-C18 100 Å, 150×30 mm from Phenomenex or RLRP-S 8p, 100 Å, 150×25 mm) as stationary phase. As mobile phase 0.1% TFA in water (A) and 0.1% TFA in ACN (B) were used which were mixed in linear binary gradients. The gradients are described as: “10 to 40% B in 30 min”, which means a linear gradient from 10% B (and correspondingly 90% A) to 40% B (and correspondingly 60% A) was run within 30 min. Flow-rates were within the range of 30 to 50 ml/min. A typical gradient for the purification of the compounds of the invention started at 5-25% B and ended after 30 min at 35-50% B and the difference between the percentage B at end and start was at least 10%. A commonly used gradient was “15 to 40% B in 30 min”.

### General Procedures for Automated/Semi-Automated Solid-Phase Synthesis:

Automated solid-phase of peptides and polyamides was performed on a Tetras Peptide Synthesizer (Advanced ChemTech) in 50 μmol and 100 μmol scales. Manual steps were performed in plastic syringes equipped with frits (material PE, Roland Vetter Laborbedarf OHG, Ammerbuch, Germany). The amount of reagents in the protocols described corresponds to the 100 μmol scale, unless stated otherwise.

Solid-phase synthesis was performed on polystyrene (cross linked with 1,4-divinylbenzene (PS) or di (ethylene glycol) dimethacrylate (DEG)), ChemMatrix (CM) or TentaGel (TG) resin. Resin linkers were trityl, wang and rink amide.

### Resin Loading:

In case of the trityl linker the attachment of the first building block (resin loading) was performed as follows. The resin (polystyrene (PS) trityl chloride, initial loading: 1.8 mmol/g) was swollen in DCM (5 ml) for 30 minutes and subsequently washed with DCM (3 ml, 1 minute). Then the resin was treated with a mixture of the corresponding building block (0.5 mmol, 5 eq.) and DIPEA (350 μl, 3.5 mmol, 35 eq.) in DCM (4 ml) for 1 hour. Afterwards the resin was washed with methanol (5 ml, 5 minutes) and DMF (3 ml, 2×1 minute).

In case of the Wang linker pre-loaded resins (polystyrene (PS) and TentaGel (TG)) were employed.

In case of the rink amide linker the attachment of the first residue the resin (CM, DEG) was performed with the same procedure as for the chain assembly as described below.

### Alloc Allyl-Deprotection:

After swelling in DMF, the resin was washed with DMF and DCM. DCM was de-oxygenated by passing a stream of nitrogen through the stirred solvent. The oxygen-free solvent was used to wash the resin trice. Then 2 ml of a 2 M solution of barbituric acid in oxygen-free DCM and 1 ml of a 25 μM solution of Tetrakis(triphenylphosphine)palladium(0) in oxygen-free DCM were added to the resin. The resin was agitated for 1 hour and then washed with DCM, MeOH, DMF, 5% DIPEA in DMF, 5% dithiocarbamate in DMF, DMF and DCM (each washing step was repeated 3 times with 3 ml, 1 minute).

### Fmoc-Deprotection:

After swelling in DMF, the resin was washed with DMF and then treated with piperidine/DMF (1:4, 3 ml, 2 and 20 minutes) and subsequently washed with DMF (3 ml, 5×1 minute).

### Dde-Deprotection:

After swelling in DMF, the resin was washed with DMF and then treated with hydrazine-hydrate/DMF (2/98, 3 ml 2×10 minutes) and subsequently washed with DMF (3 ml, 5×1 minute).

### Mtt-Deprotection:

After swelling in DCM, the resin was washed with DCM and then treated with HFIP/DCM (7/3, 4-6 ml, 4 hours) and subsequently washed with DCM (3 ml, 3×1 minute), DMF (3 ml, 3×1 ml) and DIPEA (0.9 M in DMF, 3 ml, 1 minute).

### Mtt O2PhiPr-deprotection:

After swelling in DCM, the resin was washed with DCM and then treated with 5% TFA, 5% TIPS in DCM (4-6 mL, 5×5 min) and subsequently washed with DCM (3 ml, 3×1 minute), DMF (3 ml, 3×1 ml) and DIPEA (0.9 M in DMF, 3 ml, 1 minute).

### Reduction of Nitro Groups on Solid Phase:

After swelling in DMF, the resin was washed with DMF and then treated with a 1M solution of SnCl₂×2 H₂O in DMF (3 mL per 100 μmol resin, 0.68 g SnCl₂×2 H₂O in 3 mL DMF) overnight. Afterward the resin was washed thoroughly with DMF.

### Solutions of Reagents:

Building Blocks (0.3 M in DMF or NMP), DIPEA (0.9 M in DMF), HATU (0.4 M in DMF), Acetic anhydride (0.75 M in DMF)

### Coupling: Coupling of Building Blocks Amino Acids (Chain Assembly):

Unless otherwise stated, coupling of building blocks was performed as follows: After subsequent addition of solutions of the corresponding building block (1.7 ml, 5 eq.), DIPEA solution (1.15 ml, 10 eq.) and HATU solution (1.25 ml, 5 eq.) the resin was shaken for 45 min. If necessary, the resin was washed with DMF (3 ml, 1 minute) and the coupling step was repeated.

### Coupling: Coupling of DOTA(tBu)₃-OH:

DOTA(tBu)₃-OH (5 eq compared to the initial resin loading, e.g. for 50 μmol resin 143.3 mg, 250 μmol) was dissolved in a 0.4 M solution of HATU in DMF (e.g. for 50 μmol resin 0.6 mL) and in a 0.9 M solution of DIPEA in DMF (e.g. for 50 μmol resin 0.65 mL). After leaving the mixture for 1 minute for pre-activation it was added to the resin. An hour later a 3.2 M solution of DIC in DMF (e.g. for 50 μmol resin 0.2 mL) was added and the gentle agitation of the resin continued for a further hour. Afterwards the resin was washed with DMF.

### Terminal Acetylation:

After addition of DIPEA solution (1.75 ml, 16 eq.) and acetic anhydride solution (1.75 ml, 13 eq.) the resin was shaken for 10 minutes. Afterwards the resin was washed with DMF (3 ml, 6×1 minutes).

Cleavage Method a: Cleavage of Protected Fragments from Trityl Resin:

After the completion of the assembly of the sequence the resin was finally washed with DCM (3 ml, 4×1 minute) and then dried in the vacuum. Then the resin was treated with HFIP/DCM (7/1, 4 ml, 4 hours) and the collected solution evaporated to dryness. The residue was purified with preparative HPLC or used without further purification.

### Cleavage Method B: Cleavage of Unprotected Fragments (Complete Resin Cleavage):

After the completion of the assembly of the sequence the resin was finally washed with DCM (3 ml, 4×1 minute), dried in the vacuum overnight and treated with TFA, EDT, water and TIPS (94/2.5/2.5/1) for 2 h (unless otherwise stated). Afterwards the cleavage solution was poured into a chilled mixture of MTBE and cyclohexane (1/1, 10-fold excess compared to the volume of cleavage solution), centrifuged at 4° C. for 5 minutes and the precipitate collected and dried in the vacuum. The residue was lyophilized from water/acetonitrile prior to purification or further modification.

### Cleavage Method C: Cleavage of Protective Groups of Peptides in Solution

The protected/partially protected compound was dissolved in TFA, water and TIPS (95/2.5/2.5) for 2 h (unless otherwise stated). Afterwards the cleavage solution was poured into a chilled mixture of MTBE and cyclohexane (1/1, 10-fold excess compared to the volume of cleavage solution), centrifuged at 4° C. for 5 minutes and the precipitate collected and dried in the vacuum. The residue was lyophilized from water/acetonitrile prior to purification or further modification.

### Cyclization Method: Dibromoxylene Cyclization

The crude peptide material was dissolved in a 1:1 mixture of ammonium bicarbonate solution (50 mM, pH=8.5) and acetonitrile. To the resulting mixture a solution of α,α′-dibromo-m-xylene in acetonitrile was added. Upon competition of the cyclization reaction which was judged by analytical LC-MS, TFA was added and the reaction solution subjected to lyophilization. The volume of solvent, amount of α,α′-dibromo-m-xylene and volume of TFA used in the reaction depended on the amount of resin used for the synthesis of the linear peptide precursor—per 50 μmol of initially used 60 mL of the solvent mixture, 14.5 mg (55 μmol) of α,α′-dibromo-m-xylene and 50 μL of TFA were used.

More relevant Fmoc-solid-phase-peptide synthesis methods are described in detail in “Fmoc Solid Phase Peptide Synthesis” Editors W. Chan, P. White, Oxford University Press, USA, 2000. Compounds were named using MestreNova version 12 Mnova IUPAC Name plugin (Mestrelab Research, S.L.), or AutoNom version 2.2 (Beilstein Informationssysteme Copyright© 1988-1998, Beilstein Institut fur Literatur der Organischen Chemie licensed to Beilstein Chemiedaten and Software GmbH), where appropriate.

General procedures for the preparation of a peptide comprising chelator-transition metal-complexes from corresponding peptides comprising uncomplexed chelator:

A 0.1 mM solution of the peptide comprising uncomplexed chelator in
0.4 M sodium acetate, pH=5 (Buffer A) (in case of In(III), Lu(III) or Ga(III) complexes) or
0.1 M ammonium acetate, pH=8 (Buffer B) (in case of Eu(III) complexes) was diluted with a solution 0.1 mM solution of the corresponding metal salt in water whereby the molar ratio of peptide to metal was adjusted to 1:3. The solution was stirred
at 50° C. for 20 minutes (also referred to herein as Condition A) (in case of In(III), Lu(III) or Ga(III) complexes) or
at room temperature overnight (also referred to herein as Condition B) (in case of Eu(III) complexes).

The solution was then applied to
HPLC purification (also referred to herein as Purification A) or
solid phase extraction (also referred to herein as Purification B).

In either case (HPLC purification or solid phase extraction) fractions containing the pure product were pooled and freeze dried.

### Preparation of Compounds:

The preparation of exemplary compounds of the invention is provided in the following examples. Unless otherwise specified, all starting materials and reagents are of standard commercial grade, and are used without further purification, or are readily prepared from such materials by routine methods. Those skilled in the art of organic synthesis will recognize in light of the instant disclosure that starting materials and reaction conditions may be varied as a matter of routine including incorporating obvious and well-known additional steps employed to produce compounds encompassed by the present invention.

### Example 2: Synthesis of Ac-Val-Tyr-[Cys(3MeBn)-Glu-pro-Asp-Trp-Leu-Thr-Trp-Ala-Cys]-Ape-NH-DOTA (3BP-3434)

50 μmol of Trityl PS resin were loaded with 1,5-Diaminopentane as described in the ‘General procedures for Automated/Semi-automated Solid-Phase Synthesis’. Thereafter the linear sequence (Ac-Val-Tyr-Cys-Glu-pro-Asp-Trp-Leu-Thr-Trp-Ala-Cys-Ape-NH₂) of the peptide was assembled. The resin was washed thoroughly and subjected to the ‘Cleavage method B’ protocol. The lyophilized remainder was subjected to ‘Cyclization method: Dibromoxylene cyclization’. Afterward an HPLC purification was performed (25 to 50% B in 30 min—Kinetex) to yield 12.45 mg (7.1 μmol, 13.3%) of the pure cyclic intermediate peptide Ac-Val-Tyr-[Cys(3MeBn)-Glu-pro-Asp-Trp-Leu-Thr-Trp-Ala-Cys]-Ape-NH₂. The latter was dissolved in DMSO (0.6 mL) and the solution neutralized by addition of DIPEA (5 μL). Then DOTA-NHS 8.2 mg (10.1 μmol) (hexafluorophosphate, TFA salt) was added and the pH value adjusted to roughly 8 by addition of DIPEA (12 μL). After completition of the reaction the product was isolated from the solution by preparative HPLC (20 to 45% B in 30 min—Kinetex) to yield 12.65 mg of the pure title compound (6.7%). HPLC: Rₜ=7.1 min. LC/TOF-MS: exact mass 2098.957 (calculated 2098.953). C₁₀₀H₁₃₈N₂₀O₂₆S₂ (MW=2100.420).

### Example 3: Synthesis of Ac-Val-Tyr-[Cys(3MeBn)-Glu-pro-Asp-Trp-Leu-Thr-Trp-Ala-Cys]-O2Oc-Lys(DOTA)-NH₂ (3BP-3474)

The sequence (Ac-Val-Tyr-Cys-Glu-pro-Asp-Trp-Leu-Thr-Trp-Ala-Cys-O2Oc-Lys(Mtt)-NH₂) of the peptide was assembled according to the ‘General procedures for Automated/Semi-automated Solid-Phase Synthesis’ in a 50 μmol scale on a Rink amide resin.

Then subsequently a ‘Mtt deprotection’ and a ‘Coupling: Coupling of DOTA(tBu)₃-OH’ as described in the ‘General procedures for Automated/Semi-automated Solid-Phase Synthesis’ were performed to liberate the 8-amino function of the C-terminal lysine residue and install the DOTA chelator on the latter position of the peptide resin. The resin was washed thoroughly and subjected to the ‘Cleavage method B’ protocol. The lyophilized remainder (linear, branched peptide Ac-Val-Tyr-Cys-Glu-pro-Asp-Trp-Leu-Thr-Trp-Ala-Cys-O2Oc-Lys(DOTA)-NH₂) was subjected to ‘Cyclization method: Dibromoxylene cyclization’. The remainder obtained after lyophilization was purified by preparative HPLC (20 to 45% B in 30 min—Kinetex) to yield 24.15 mg of the pure title compound (11.1%). HPLC: Rₜ=6.8 min. LC/TOF-MS: exact mass 2287.037 (calculated 2287.033). C₁₀₇H₁₅₀N₂₂O₃₀S₂ (MW=2288.602).

### Example 4: Synthesis of Ac-Val-Tyr-[Cys(3MeBn)-Glu-pro-Asp-Trp-Leu-Thr-Trp-Glu(NH-Apr-DOTA)-Cys]-NH₂ (3BP-3478)

The sequence (Ac-Val-Tyr-Cys-Glu-pro-Asp-Trp-Leu-Thr-Trp-Glu(OAll)-Cys-NH₂) of the peptide was assembled according to the ‘General procedures for Automated/Semi-automated Solid-Phase Synthesis’ in a 50 μmol scale on a Rink amide resin. The allyl protecting group on the glutamic acid side chain was removed by executing an ‘Alloc Allyl-deprotection’. After the liberated carboxylic acid moiety had been transformed into an Oxyma active ester by addition of Oxyma (35.7 mg, 250 μmol), DIC (38.7 μL, 250 μmol) and DIPEA (51.4 μL, 300 μmol), N-1-Fmoc-1,3-diaminopropane (HCl salt) (83.25 mg, 250 μmol) was added and the resin agitated at 50° C. for 1 hour. Another portion of DIC was added and the resin agitated for additional 30 minutes at 50° C. The Fmoc group was removed and the DOTA chelator installed by execution of the ‘Coupling: Coupling of DOTA(tBu)₃-OH’ as described in the ‘General procedures for Automated/Semi-automated Solid-Phase Synthesis’. The resin was washed thoroughly and subjected to the ‘Cleavage method B’ protocol. The lyophilized remainder was subjected to ‘Cyclization method: Dibromoxylene cyclization’. The remainder obtained after lyophilization was purified by preparative HPLC (20 to 45% B in 30 min—Kinetex) to yield 11.84 mg of the pure title compound (5.8%). HPLC: Rₜ=7.0 min. LC/TOF-MS: exact mass 2127.951 (calculated 2127.943). C₁₀₀H₁₃₇N₂₁O₂₇S₂ (MW=2129.418).

### Example 5: Synthesis of Ac-Val-Tyr-[Cys(3MeBn)-Glu-Nlys(DOTA)-Asp-Trp-Leu-Thr-Trp-Ala-Cys]-NH₂ (3BP-3562)

The sequence (Ac-Val-Tyr-Cys-Glu-Nlys-Asp-Trp-Leu-Thr-Trp-Ala-Cys-NH₂) of the peptide was assembled according to the ‘General procedures for Automated/Semi-automated Solid-Phase Synthesis’ in a 50 μmol scale on a Rink amide resin. The resin was washed thoroughly and subjected to the ‘Cleavage method B’ protocol. The lyophilized remainder was subjected to ‘Cyclization method: Dibromoxylene cyclization’. Afterward an HPLC purification was performed (20 to 45% B in 30 min—Kinetex) to yield 43.13 mg (26 μmol, 52.0%) of the pure cyclic intermediate peptide Ac-Val-Tyr-[Cys(3MeBn)-Glu-Nlys-Asp-Trp-Leu-Thr-Trp-Ala-Cys]-NH₂. The latter was dissolved in DMSO (0.9 mL) and the solution neutralized by addition of DIPEA (9 μL). Then DOTA-NHS 23.7 mg (31.2 μmol) (Hexafluorophosphate, TFA salt) was added and the pH value adjusted to roughly 8 by addition of DIPEA (18 μL). After competition of the reaction the product was isolated from the solution by preparative HPLC (20 to 45% B in 30 min—Kinetex) to yield 32.31 mg of the pure title compound (17.5%). HPLC: Rₜ=7.0 min. LC/TOF-MS: exact mass 2044.910 (calculated 2044.906). C₉₆H₁₃₂N₂₀O₂₆S₂ (MW=2288.602).

### Example 6: Synthesis of DOTA-APAc-Val-Tyr-[Cys(3MeBn)-Glu-pro-Asp-Trp-Leu-Thr-Trp-Ser-Cys]-NH₂ (3BP-3583)

The sequence (DOTA-APAc-Val-Tyr-Cys-Glu-pro-Asp-Trp-Leu-Thr-Trp-Ser-Cys-NH₂) of the peptide was assembled according to the ‘General procedures for Automated/Semi-automated Solid-Phase Synthesis’ in a 50 μmol scale on a Rink amide resin. The N-terminal DOTA was coupled as described in the general procedures section (‘Coupling: Coupling of DOTA(tBu)₃-OH’). After performing the steps of ‘Cleavage method B’ the lyophilized crude peptide residue was subjected to ‘Cyclization method: Dibromoxylene cyclization’. The remainder obtained after lyophilization was purified by preparative HPLC (20 to 45% B in 30 min—Kinetex) to yield 23.0 mg of the pure title compound (11.4%). HPLC: Rt=6.8 min.

LC/TOF-MS: exact mass 2127.951 (calculated 2127.943). C₁₀₀H₁₃₇N₂₁O₂₇S₂ (MW=2129.418).

### Example 7: Synthesis of DOTA-Tyr-[Cys(3MeBn)-Glu-pro-{Lys-Trp-Leu-Glu}-Trp-Ser-Cys]-NH₂ (3BP-3934)

The sequence (DOTA-Tyr-Cys-Glu-pro-Lys(Alloc)-Trp-Leu-Glu(OAll)-Trp-Ser-Cys-NH₂) of the peptide was assembled according to the ‘General procedures for Automated/Semi-automated Solid-Phase Synthesis’ in a 100 μmol scale on a Rink amide resin. The N-terminal DOTA was coupled as described in the general procedures section (‘Coupling: Coupling of DOTA(tBu)₃-OH’). The allyloxycarbonyl protecting group (Alloc) on the the lysine side chain and the allyl protecting group on the glutamic acid side chain were removed simultaneously by executing an ‘Alloc Allyl-deprotection’ as described in the ‘General procedures’ section.

The liberated amino and carboxylic acid function were intramolecularly connected on resin by forming an amide functionality as follows: After addition of Oxyma (28.4 mg, 200 μmol) and DIC (31 μL, 200 μmol) the resin was gently agitated overnight. The resin was washed thoroughly and subjected to the ‘Cleavage method B’ protocol. The crude intermediate macro lactame (DOTA-Tyr-Cys-Glu-pro-{Lys-Trp-Leu-Glu}-Trp-Ser-Cys-NH₂) obtained after lyophilization was subjected to ‘Cyclization method: Dibromoxylene cyclization’. After lyophilization the product was purified by preparative HPLC (20 to 45% B in 30 min—Kinetex) to yield 12.20 mg of the pure title compound (12.2%). HPLC: Rₜ=6.6 min. LC/TOF-MS: exact mass 1911.835 (calculated 1911.832). C₉₁H₁₂₁N₁₉O₂₃S₂ (MW=1913.184).

### Example 8: Synthesis of DOTA-APAc-Val-Tyr-[Cys(3MeBn)-Glu-pro-Glu(AGLU)-Trp-Leu-Thr-Trp-Ser-Cys]-NH₂ (3BP-4120)

The sequence (DOTA-APAc-Val-Tyr-Cys(Mmt)-Glu-pro-Glu(OAll)-Trp-Leu-Thr-Trp-Ser-Cys(Mmt)-NH₂) of the peptide was assembled according to the ‘General procedures for Automated/Semi-automated Solid-Phase Synthesis’ in a 100 μmol scale on a Rink amide resin. The N-terminal DOTA was coupled as described in the general procedures section (‘Coupling: Coupling of DOTA(tBu)₃-OH’). The allyl protecting group on glutamic acid was removed by executing an ‘Alloc Allyl-deprotection’. Aglu was coupled to the acid as follows: a mixture of the AGLU building block (98 mg, 375 μmol, 3.75 eq.), Oxyma (53 mg, 375 μmol, 3.75 eq.) and DIC (58 μl, 375 μmol, 3.75 eq.) in 1.7 mL DMF was added to the resin and the mixture was gently agitated at 50° C. for 90 min before the same amount of DIC was added again. Agitation was continued for 90 min at 50° C. The resin was washed thoroughly and subjected to the ‘Cleavage method B’ protocol. The lyophilized remainder was subjected to ‘Cyclization method: Dibromoxylene cyclization’. The remainder obtained after lyophilization was purified by preparative HPLC (25 to 45% B in 30 min—Kinetex) to yield 6.64 mg of the pure title compound (2.88%). HPLC: Rt=6.612 min. LC/TOF-MS: exact mass 2305.0497 (calculated 2305.0435). C₁₀₇H152N₂₂₀₃₁S2 (MW=2306.617).

### Example 9: Synthesis of DOTA-APAc-Val-{Asp-[Cys(3MeBn)-Glu-pro-Asp-Trp-Leu-Thr-Trp-Dap}-Cys]-NH₂ (3BP-4174)

The sequence (DOTA-APAc-Val-Asp(O2PhiPr)-Cys(StBu)-Glu-pro-Asp-Trp-Leu-Thr-Trp-Dap(Mtt)-Cys(StBu)-NH₂) of the peptide was assembled according to the ‘General procedures for Automated/Semi-automated Solid-Phase Synthesis’ in a 100 μmol scale on a Rink amide resin. The N-terminal DOTA was coupled as described in the general procedures section (‘Coupling: Coupling of DOTA(tBu)₃-OH’). The 2-Phenyl-iso-propyl group (O2PhiPr) on the aspartic acid side chain and the methyl trityl (Mtt) on the diamino propionic acid (Dap) side chain were removed simultaneously by executing a ‘Mtt/O2PhiPr-deprotection’ as described in the ‘General procedures’ section. The liberated amino and carboxylic acid function were coupled on resin to from an amide as follows: After addition of Oxyma (28.4 mg, 200 μmol) and DIC (31 μL, 200 μmol) the resin was gently agitated overnight. The cysteine side chains were released from the StBu protecting groups by overnight treatment of the resin with a solution of DMF, water, DIPEA and 1,4-Dithio-DL-threitol (DTT) (3 mL, 9:1:0.2:1). The resin was washed thoroughly and subjected to the ‘Cleavage method B’ protocol. The crude intermediate lactam (DOTA-APAc-Val-{Asp-Cys-Glu-pro-Asp-Trp-Leu-Thr-Trp-Dap}-Cys-NH₂) obtained after lyophilization was subjected to ‘Cyclization method: Dibromoxylene cyclization’. After lyophilization the product was purified by preparative HPLC (20 to 45% B in 30 min—Kinetex) to yield 3.82 mg of the pure title compound (2.3%). HPLC: Rₜ=7.6 min. LC/TOF-MS: exact mass 2060.9182 (calculated 2060.912). C₉₅H132N₂₂₀₂₆S2 (MW=2062.332).

### Example 10: Synthesis of DOTA-APAc-Val-Tyr-[Cys(3MeBn)-Glu-pro-Glu(NHMe2Nph)-Trp-Leu-Thr-Trp-Ser-Cys]-NH₂ (3BP-4215)

The sequence (DOTA-APAc-Val-Tyr-Cys-Glu-pro-Glu(OAll)-Trp-Leu-Thr-Trp-Ser-Cys-NH₂) of the peptide was assembled according to the ‘General procedures for Automated/Semi-automated Solid-Phase Synthesis’ in a 50 μmol scale on a Rink amide resin. The N-terminal DOTA was coupled as described in the general procedures section (‘Coupling: Coupling of DOTA(tBu)₃-OH’). The allyl protecting group on the glutamic acid side chain was removed by executing an ‘Alloc Allyl-deprotection’. After the liberated carboxylic acid moiety had been transformed into an Oxyma active ester by addition of Oxyma (35.7 mg, 250 μmol), DIC (38.7 μL, 250 μmol) and DIPEA (51.4 μL, 300 μmol), 1-(naphthalen-2-yl)methanamine (39.25 mg, 250 μmol) was added and the resin agitated at 50° C. for 1 hour. Another portion of DIC was added and the resin agitated for additional 30 minutes at 50° C. The resin was washed thoroughly and subjected to the ‘Cleavage methodB’ protocol. The lyophilized remainder was subjected to ‘Cyclization method: Dibromoxylene cyclization’. The remainder obtained after lyophilization was purified by preparative HPLC (25 to 50% B in 30 min—Kinetex) to yield 21.88 mg of the pure title compound (10.1%). HPLC: Rₜ=7.9 min. LC/TOF-MS: exact mass 2281.053 (calculated 2281.038). C₁₁₂H₁₄₈N₂₂O₂₆S₂ (MW=2282.642).

### Example 11: Synthesis of DOTA-APAc-Val-Tyr-[Cys(3MeBn)-Glu-pro-Asp-Af3(HO-Succinyl)-Leu-Thr-Trp-Ser-Cys]-NH₂ (3BP-4237)

The sequence (DOTA-APAc-Val-Tyr-Cys-Glu-pro-Asp-Nf3-Leu-Thr-Trp-Ser-Cys-NH₂) of the peptide was assembled according to the ‘General procedures for Automated/Semi-automated Solid-Phase Synthesis’ in a 50 μmol scale on a Rink amide resin. The N-terminal DOTA was coupled as described in the general procedures section (‘Coupling: Coupling of DOTA(tBu)₃-OH’). The nitro moiety of the Nf3 building block was transformed into an amino function (Af3) by executing the ‘Reduction of Nitro groups on solid phase’ procedure described in the ‘General procedures for Automated/Semi-automated Solid-Phase Synthesis’ section. The resulting amino function was acylated by coupling Mono-tert-butyl succinate. The resin was washed thoroughly and subjected to the ‘Cleavage method B’ protocol. The lyophilized remainder was subjected to ‘Cyclization method: Dibromoxylene cyclization’. The remainder obtained after lyophilization was purified by preparative HPLC (25 to 45% B in 30 min—Kinetex) to yield 14.04 mg of the pure title compound (6.7%). HPLC: Rₜ=6.2 min. LC/TOF-MS: exact mass 2203.967 (calculated 2203.959). C₁₀₂H₁₄₁N₂₁O₃₀S₂ (MW=2205.47).

### Example 12: Synthesis of DOTA-PPAc-Gln-[Cys(3MeBn)-Glu-pro-Asp-Af3(Cpsu)-Leu-Thr-Trp-Ser-Cys]-NH₂ (3BP-4452)

The sequence (DOTA-PPAc-Gln-Cys-Glu-pro-Asp-Nf3-Leu-Thr-Trp-Ser-Cys-NH₂) of the peptide was assembled according to the ‘General procedures for Automated/Semi-automated Solid-Phase Synthesis’ in a 50 μmol scale on a Rink amide resin. The N-terminal DOTA was coupled as described in the general procedures section (‘Coupling: Coupling of DOTA(tBu)₃-OH’). The nitro moiety of the Nf3 building block was transformed into an amino function (Af3) by executing the ‘Reduction of Nitro groups on solidphase’ procedure described in the ‘General procedures for Automated/Semi-automated Solid-Phase Synthesis’ section. The resulting amino function was acylated by addition of 3-carboxypropanesulfonamide (41.8 mg, 0.25 mmol, 5 eq.), HATU (95.1 mg, 0.25 mmol, 5 eq.) and DIPEA (85.6 μl, 0.5 mmol, 10 eq.) in 1.5 mL DMF. The reaction was allowed to proceed under gentle agitation at RT overnight. The resin was washed thoroughly and subjected to the ‘Cleavage method B’ protocol. The lyophilized remainder was subjected to ‘Cyclization method: Dibromoxylene cyclization’. The remainder obtained after lyophilization was purified by preparative HPLC (15 to 35% B in 20 min—Kinetex) to yield 14.00 mg of the pure title compound (13.3%). HPLC: Rt=5.69 min. LC/TOF-MS: exact mass 2104.9006 (calculated 2104.8693). C92H132N22O29S3 (MW=2106.364).

### Example 13: Synthesis of DOTA-Gln-[Cys(3MeBn)-Glu-pro-Asp-Aph(SaPr)-Leu-Thr-Trp-Ser-Cys]-NH₂ (3BP-4501)

The sequence (DOTA-Gln-Cys-Glu-pro-Asp-Nif-Leu-Thr-Trp-Ser-Cys-NH₂) of the peptide was assembled according to the ‘General procedures for Automated/Semi-automated Solid-Phase Synthesis’ in a 50 μmol scale on a Rink amide resin. The N-terminal DOTA was coupled as described in the general procedures section (‘Coupling: Coupling of DOTA(tBu)₃-OH’). The nitro moiety of the Nif building block was transformed into an amino function (Aph) by executing the ‘Reduction of Nitro groups on solidphase’ procedure described in the ‘General procedures for Automated/Semi-automated Solid-Phase Synthesis’ section. The resulting amino function was acylated by addition of 3-sufamoylpropanoic acid (38.3 mg, 0.25 mmol, 5 eq.), HATU (95.1 mg, 0.25 mmol, 5 eq.) and DIPEA (85.6 μl, 0.5 mmol, 10 eq.) in 1.5 mL DMF. The reaction was allowed to proceed under gentle agitation for 5 h at RT. The resin was washed thoroughly and subjected to the ‘Cleavage method B’ protocol. The lyophilized remainder was subjected to ‘Cyclization method: Dibromoxylene cyclization’. The remainder obtained after lyophilization was purified by preparative HPLC (15 to 40% B in 20 min—Kinetex) to yield 9.95 mg of the pure title compound (10.12%). HPLC: Rt=5.633 min. LC/TOF-MS: exact mass 1964.7734 (calculated 1964.7743). C85H120N20O28S3 (MW=1966.181).

### Example 14: Synthesis of DOTA-{Glu-[Cys(3MeBn)-Glu-pro-Asp-Af3(Cpsu)-Leu-Thr-Trp-Dap}-Cys]-NH₂ (3BP-4503)

The sequence (H-Glu(O2PhiPr)-Cys(SDmp)-Glu-pro-Asp-Nf3-Leu-Thr-Trp-Dap(Mtt)-Cys(SDmp)-NH₂) of the peptide was assembled according to the ‘General procedures for Automated/Semi-automated Solid-Phase Synthesis’ in a 50 μmol scale on a low loaded Rink amide resin. Alloc protection was achieved by adding allylchloroformate (48 μL, 450 μmol, 9 eq.) and DIPEA (77 μL, 450 μmol, 9 eq.) in DCM (4 mL) to the resin-bound peptide followed by gentle agitation for 4 h at RT. The solution was removed and the resin was washed thoroughly with DCM. For SDmp deprotection, the resin-bound peptide was treated with a solution of 20% β-mercaptoethanol in 0.1 M NMM in DMF (2.5 mL) for 2.5 h. The resin was washed thoroughly with DMF. The peptide was subjected to on-resin cyclization by addition of α,α′-dibromo-m-xylene (60 μmol, 15.8 mg, 1.2 eq.) and DIPEA (250 μmol, 42.8 μL, 5 eq.) in DMF (1.2 mL). The reaction was allowed to proceed under gentle agitation at 50° C. for 90 min. The solvent was removed and the procedure was repeated for 30 min to achieve complete conversion. The resin was washed thoroughly with DMF. The 2-phenyl-iso-propyl group (O2PhiPr) on the glutamic acid side chain and the methyl trityl (Mtt) on the diamino propionic acid (Dap) side chain were removed simultaneously by executing a ‘Mtt O2PhiPr-deprotection’ as described in the ‘General procedures’ section. The liberated amino and carboxylic acid functionalities were coupled on resin to form an amide as follows: A solution of DEPBT (29.9 mg, 0.1 mmol, 2 eq.) and DIPEA (17.4 μL, 0.1 mmol, 2 eq.) was added to the resin and the reaction was allowed to proceed overnight at room temperature under gentle agitation. The resin was washed several times with DMF. The nitro moiety of the Nf building block was transformed into an amino function (Af3) by executing the ‘Reduction of Nitro groups on solid phase’ procedure described in the ‘General procedures for Automated/Semi-automated Solid-Phase Synthesis’ section. The resulting amino function was acylated by addition of 3-carboxypropanesulfonamide (41.8 mg, 0.25 mmol, 5 eq.), HATU (95.1 mg, 0.25 mmol, 5 eq.) and DIPEA (85.6 μl, 0.5 mmol, 10 eq.) in 1.5 mL DMF. The reaction was allowed to proceed under agitation at RT for 5 h followed by DMF washes. The alloc protecting group on glutamic acid was removed by executing an ‘Alloc Allyl-deprotection’. The N-terminal DOTA was coupled as described in the general procedures section (‘Coupling: Coupling of DOTA(tBu)₃-OH’). The resin was washed thoroughly and subjected to the ‘Cleavage method B’ protocol. The crude peptide was obtained after lyophilization and purified by preparative HPLC (15 to 40% B in 20 min—Kinetex) to yield 5.18 mg of the pure title compound (5.3%). HPLC: Rt=6.21 min. LC/TOF-MS: exact mass 1960.7865 (calculated 1960.7794). C86H120N20O27S3 (MW=1962.192).

### Example 15: FACS Binding Assay

In order to determine binding of compounds according to the present invention to CAIX-expressing cells, a FACS binding assay was established.

CAIX-expressing human HT-29 colorectal cancer cells (DSMZ, RRID: CVCL_0320) were cultured in McCoys's 5A modified medium (Biochrom, #F1015) including 10% fetal calf serum (FCS), 2 mM L-glutamine, 100 U/ml penicillin and 100 μg/mL streptomycin. Cells were detached with Accutase (Biolegend, #423201) and washed in FACS buffer (PBS including 1% FCS). Cells were diluted in FACS buffer to a final concentration of 500.000 cells per ml. 200 μL of the cell suspension were transferred to a u-shaped non-binding 96-well plate (Greiner) and cells were washed in ice-cold FACS buffer.

For EC₅₀ determination, cells were incubated with various concentrations of biotinylated or fluorophore-labeled compound at 4° C. for 1 hour. For IC₅₀ determination, cells were incubated with 10 nM biotin-labeled 3BP-2776 (H-Met-Val-Tyr-Cys([3MeBn)-Glu-Gly-Asp-Trp-Leu-Thr-Trp-Gln-Cys]-Ttds-Lys(Bio)-NH₂) or 3 nM Cy5-labeled 3BP-4149 (Cy5SO3-APAc-Val-Tyr-[Cys(3MeBn)-Glu-pro-Asp-Trp-Leu-Thr-Trp-Ser-Cys]-NH₂) in the presence of increasing concentrations of non-labeled test compounds at 4° C. for 1 hour. When biotinylated compounds were used an additional incubation step with 1 μg/mL APC-Streptavidin (Miltenyi; #130-106-791) in 50 μL FACS buffer for 30 minutes on ice was performed.

Cells were washed twice with ice-cold FACS buffer and analyzed in an Attune NxT flow cytometer (Thermo Fisher). Median fluorescence intensities (MFI) of the APC/Cy5-channel were calculated by Attune NxT software. MFI values were plotted against peptide concentration and four parameter logistic (4PL) curve fitting and EC₅₀/pEC₅₀ or IC₅₀/pIC₅₀ calculations were performed using ActivityBase software.

The results of the pEC₅₀ assay are shown in Table 9 and the results of the pIC₅₀ assay in Table 10. pEC₅₀ category A stands for pEC₅₀ values >8.0, category B for pEC₅₀ values between 7.1 and 8.0, and category C for pEC₅₀ values between 6.1 and 7.0. pIC₅₀ category A stands for pIC₅₀ values >8.0, category B for pIC₅₀ values between 7.1 and 8.0, and category C for pIC₅₀ values between 6.1 and 7.0.

TABLE 9
Compound ID, sequence, exact calculated mass, exact mass found, retention time in
minutes as determined by HPLC and pEC₅₀ category of FACS binding assay.

| | | Exact | Exact | | PEC₅₀ |
|---|---|---|---|---|---|
| | | mass | mass | Rt | category |
| ID | Sequence | (calc) | (found) | (HPLC) | (activity) |
| 3BP- | H-Met-Gln-[Cys(3MeBn)-Glu-Ile-Trp-Val-Asp-Gly-Trp-Val- | 2326.066 | 2326.073 | 7.0 | B |
| 2775 | Thr-Cys]-Ttds-Lys(Bio)-NH2 | | | | |
| 3BP- | H-Met-Val-Tyr-[Cys(3MeBn)-Glu-Gly-Asp-Trp-Leu-Thr-Trp- | 2390.061 | 2390.068 | 6.6 | A |
| 2776 | Gln-Cys]-Ttds-Lys(Bio)-NH2 | | | | |
| 3BP- | H-Met-Val-Tyr-[Cys(3MeBn)-Glu-Gly-Ser-Trp-Leu-Gly-Trp- | 2277.013 | 2277.018 | 6.8 | A |
| 2777 | Ser-Cys]-Ttds-Lys(Bio)-NH2 | | | | |
| 3BP- | H-Met-Arg-[Cys(3MeBn)-Glu-Ile-Trp-Val-Asp-Gly-Trp-Val- | 2368.088 | 2368.091 | 6.8 | A |
| 2778 | Asp-Cys]-Ttds-Lys(Bio)-NH2 | | | | |
| 3BP- | Cy5SO3-APAc-Val-Tyr-[Cys(3MeBn)-Glu-pro-Asp-Trp- | 2329.058 | 2329.075 | 9.7 | A |
| 4149 | Leu-Thr-Trp-Ser-Cys]-NH2 | | | | |

TABLE 10
Compound ID, sequence, exact calculated mass, exact mass found, retention time in
minutes as determined by HPLC, Tracer ID and pIC₅₀ category of FACS binding assay.

| | | | | | | pIC₅₀ |
|---|---|---|---|---|---|---|
| | | | | | | cate- |
| | | Exact | Exact | | | gory |
| | | mass | mass | Rₜ | Tracer | (activ- |
| ID | Sequence | (calc) | (found) | (HPLC) | ID | ity) |
| 3BP- | Ac-Met-Val-Tyr-[Cys(3MeBn)-Glu-Gly-Asp-Trp-Leu-Thr-Trp- | 1775.715 | 1775.718 | 6.4 | 3BP- | C |
| 2836 | Gln-Cys]-NH2 | | | | 2776 | |
| 3BP- | Ac-Val-Tyr-[Cys(3MeBn)-Glu-Gly-Asp-Trp-Leu-Thr-Trp-Gln- | 1644.674 | 1644.678 | 7.3 | 3BP- | A |
| 2837 | Cys]-NH2 | | | | 2776 | |
| 3BP- | Ac-Val-Tyr-[Cys(3MeBn)-Glu-Gly-Asp-Trp-Leu-Thr-Trp-Gln- | 2461.133 | 2461.138 | 6.7 | 3BP- | B |
| 2949 | Cys]-Ttds-lys(DOTA)-NH2 | | | | 2776 | |
| 3BP- | DOTA-Ttds-Val-Tyr-[Cys(3MeBn)-Glu-Gly-Asp-Trp-Leu-Thr- | 2291.028 | 2291.040 | 6.7 | 3BP- | A |
| 2959 | Trp-Gln-Cys]-NH2 | | | | 2776 | |
| 3BP- | Ac-val-Tyr-[Cys(3MeBn)-Glu-Gly-Asp-Trp-Leu-Thr-Trp-Gln- | 1644.674 | 1644.677 | 7.4 | 3BP- | B |
| 3043 | Cys]-NH2 | | | | 2776 | |
| 3BP- | Ac-Val-tyr-[Cys(3MeBn)-Glu-Gly-Asp-Trp-Leu-Thr-Trp-Gln- | 1644.674 | 1644.677 | 7.4 | 3BP- | C |
| 3044 | Cys]-NH2 | | | | 2776 | |
| 3BP- | Ac-Val-Tyr-[Cys(3MeBn)-glu-Gly-Asp-Trp-Leu-Thr-Trp-Gln- | 1644.674 | 1644.677 | 7.1 | 3BP- | C |
| 3045 | Cys]-NH2 | | | | 2776 | |
| 3BP- | Ac-Val-Tyr-[Cys(3MeBn)-Glu-ala-Asp-Trp-Leu-Thr-Trp-Gln- | 1658.690 | 1658.692 | 7.4 | 3BP- | A |
| 3046 | Cys]-NH2 | | | | 2776 | |
| 3BP- | Ac-Val-Tyr-[Cys(3MeBn)-Glu-Gly-asp-Trp-Leu-Thr-Trp-Gln- | 1644.674 | 1644.675 | 7.1 | 3BP- | C |
| 3047 | Cys]-NH2 | | | | 2776 | |
| 3BP- | Ac-Val-Tyr-[Cys(3MeBn)-Glu-Gly-Asp-Trp-leu-Thr-Trp-Gln- | 1644.674 | 1644.672 | 7.4 | 3BP- | C |
| 3048 | Cys]-NH2 | | | | 2776 | |
| 3BP- | Ac-Val-Tyr-[Cys(3MeBn)-Glu-Gly-Asp-Trp-Leu-thr-Trp-Gln- | 1644.674 | 1644.679 | 7.3 | 3BP- | C |
| 3049 | Cys]-NH2 | | | | 2776 | |
| 3BP- | Ac-Val-Tyr-[Cys(3MeBn)-Ala-Gly-Asp-Trp-Leu-Thr-Trp-Gln- | 1586.669 | 1586.672 | 7.8 | 3BP- | A |
| 3052 | Cys]-NH2 | | | | 2776 | |
| 3BP- | Ac-Val-Tyr-[Cys(3MeBn)-Glu-Gly-Ala-Trp-Leu-Thr-Trp-Gln- | 1600.684 | 1600.684 | 7.4 | 3BP- | B |
| 3053 | Cys]-NH2 | | | | 2776 | |
| 3BP- | Ac-Val-Tyr-[Cys(3MeBn)-Glu-Gly-Asp-Trp-Ala-Thr-Trp-Gln- | 1602.627 | 1602.626 | 6.5 | 3BP- | C |
| 3055 | Cys]-NH2 | | | | 2776 | |
| 3BP- | Ac-Val-Tyr-[Cys(3MeBn)-Glu-Gly-Asp-Trp-Leu-Ala-Trp-Gln- | 1614.664 | 1614.665 | 7.2 | 3BP- | B |
| 3056 | Cys]-NH2 | | | | 2776 | |
| 3BP- | Ac-Val-Tyr-[Cys(3MeBn)-Glu-Gly-Asp-Trp-Leu-Thr-Trp-Ala- | 1587.653 | 1587.656 | 7.7 | 3BP | A |
| 3058 | Cys]-NH2 | | | | 2776 | |
| 3BP- | Ac-Val-Tyr-[Cys(3MeBn)-Glu-Aib-Asp-Trp-Leu-Thr-Trp-Gln- | 1672.705 | 1672.709 | 8.0 | 3BP- | A |
| 3059 | Cys]-NH2 | | | | 2776 | |
| 3BP- | Ac-Val-Tyr-[Cys(3MeBn)-Glu-Nmg-Asp-Trp-Leu-Thr-Trp-Gln- | 1658.690 | 1658.695 | 7.3 | 3BP- | A |
| 3060 | Cys]-NH2 | | | | 2776 | |
| 3BP- | Ac-Val-Tyr-[Cys(3MeBn)-Glu-Gly-Aib-Trp-Leu-Thr-Trp-Gln- | 1614.700 | 1614.705 | 7.5 | 3BP- | C |
| 3061 | Cys]-NH2 | | | | 2776 | |
| 3BP- | Ac-Val-Tyr-[Pen(3MeBn)-Glu-Gly-Asp-Trp-Leu-Thr-Trp-Gln- | 1672.705 | 1672.711 | 7.5 | 3BP- | B |
| 3063 | Cys]-NH2 | | | | 2776 | |
| 3BP- | Ac-Val-Tyr-[Cys(3MeBn)-Glu-Gly-Asp-Trp-Leu-Thr-Trp-Gln- | 1672.705 | 1672.711 | 8.0 | 3BP- | A |
| 3064 | Pen]-NH2 | | | | 2776 | |
| 3BP- | Ac-Val-Tyr-[Cys(3MeBn)-Glu-Gly-Asp-Trp-Npg-Thr-Trp-Gln- | 1658.690 | 1658.695 | 7.6 | 3BP- | A |
| 3065 | Cys]-NH2 | | | | 2776 | |
| 3BP- | Ac-Val-Tyr-[Cys(3MeBn)-Glu-Gly-Asp-Trp-Tle-Thr-Trp-Gln- | 1644.674 | 1644.679 | 6.8 | 3BP- | B |
| 3066 | Cys]-NH2 | | | | 2776 | |
| 3BP- | Hex-Val-Tyr-[Cys(3MeBn)-Glu-Gly-Asp-Trp-Leu-Thr-Trp- | 1700.737 | 1700.737 | 8.7 | 3BP- | A |
| 3107 | Gln-Cys]-NH2 | | | | 2776 | |
| 3BP- | Bz-Val-Tyr-[Cys(3MeBn)-Glu-Gly-Asp-Trp-Leu-Thr-Trp-Gln- | 1706.690 | 1706.693 | 8.3 | 3BP- | A |
| 3108 | Cys]-NH2 | | | | 2776 | |
| 3BP- | Pha-Val-Tyr-[Cys(3MeBn)-Glu-Gly-Asp-Trp-Leu-Thr-Trp- | 1720.705 | 1720.705 | 8.4 | 3BP | A |
| 3109 | Gln-Cys]-NH2 | | | | 2776 | |
| 3BP- | Php-Val-Tyr-[Cys(3MeBn)-Glu-Gly-Asp-Trp-Leu-Thr-Trp- | 1734.721 | 1734.723 | 8.7 | 3BP- | A |
| 3110 | Gln-Cys]-NH2 | | | | 2776 | |
| 3BP- | Iva-Val-Tyr-[Cys(3MeBn)-Glu-Gly-Asp-Trp-Leu-Thr-Trp- | 1686.721 | 1686.700 | 8.2 | 3BP- | A |
| 3111 | Gln-Cys]-NH2 | | | | 2776 | |
| 3BP- | DOTA-Ttds-Val-Tyr-[Cys(3MeBn)-Glu-ala-Asp-Trp-Leu-Thr- | 2305.044 | 2305.060 | 6.8 | 3BP- | A |
| 3270 | Trp-Gln-Cys]-NH2 | | | | 2776 | |
| 3BP- | DOTA-Ttds-Val-Tyr-[Cys(3MeBn)-Glu-Nmg-Asp-Trp-Leu- | 2305.044 | 2305.044 | 7.6 | 3BP- | A |
| 3271 | Thr-Trp-Gln-Cys]-NH2 | | | | 2776 | |
| 3BP- | DOTA-Ttds-Val-Tyr-[Cys(3MeBn)-Glu-Gly-Asp-Trp-Leu-Thr- | 2234.006 | 2234.020 | 7.0 | 3BP- | A |
| 3272 | Trp-Ala-Cys]-NH2 | | | | 2776 | |
| 3BP- | DOTA-Ttds-Val-Tyr-[Cys(3MeBn)-Glu-Nmg-Asp-Trp-Leu- | 2248.022 | 2248.031 | 7.1 | 3BP- | B |
| 3278 | Thr-Trp-Ala-Cys]-NH2 | | | | 2776 | |
| 3BP- | DOTA-Ttds-Val-Tyr-[Cys(3MeBn)-Glu-ala-Asp-Trp-Leu-Thr- | 2248.022 | 2248.030 | 7.0 | 3BP- | A |
| 3279 | Trp-Ala-Cys]-NH2 | | | | 2776 | |
| 3BP- | Ac-Val-Tyr-[Cys(3MeBn)-Glu-ala-Asp-Trp-Leu-Thr-Trp-Ala- | 1601.668 | 1601.677 | 7.8 | 3BP- | A |
| 3280 | Cys]-NH2 | | | | 2776 | |
| 3BP- | Ac-Val-Tyr-[Cys(3MeBn)-Glu-nma-Asp-Trp-Leu-Thr-Trp-Ala- | 1615.684 | 1615.689 | 7.8 | 3BP- | A |
| 3281 | Cys]-NH2 | | | | 2776 | |
| 3BP- | Ac-Val-Tyr-[Cys(3MeBn)-Glu-pro-Asp-Trp-Leu-Thr-Trp-Ala- | 1627.684 | 1627.689 | 7.8 | 3BP- | A |
| 3282 | Cys]-NH2 | | | | 2776 | |
| 3BP- | Ac-Val-Tyr-[Cys(3MeBn)-Glu-pip-Asp-Trp-Leu-Thr-Trp-Ala- | 1641.700 | 1641.703 | 8.2 | 3BP- | A |
| 3283 | Cys]-NH2 | | | | 2776 | |
| 3BP- | Ac-Val-Tyr-[Cys(3MeBn)-Glu-ala-Asp-Trp-Leu-Thr-Trp-Gly- | 1587.653 | 1587.656 | 7.4 | 3BP- | A |
| 3284 | Cys]-NH2 | | | | 2776 | |
| 3BP- | Ac-Val-Tyr-[Cys(3MeBn)-Glu-Nmg-Asp-5Clw-Leu-Thr-Trp- | 1692.651 | 1692.657 | 7.9 | 3BP- | A |
| 3289 | Gln-Cys]-NH2 | | | | 2776 | |
| 3BP- | Ac-Val-Tyr-[Cys(3MeBn)-Glu-Nmg-Asp-Egc-Leu-Thr-Trp- | 1672.705 | 1672.710 | 8.1 | 3BP- | A |
| 3290 | Gln-Cys]-NH2 | | | | 2776 | |
| 3BP- | Ac-Val-Tyr-[Cys(3MeBn)-Glu-Nmg-Asp-Trp-Leu-Thr-Trp-Gly- | 1587.653 | 1587.658 | 7.4 | 3BP | A |
| 3304 | Cys]-NH2 | | | | 2776 | |
| 3BP- | Ac-Val-Tyr-[Cys(3MeBn)-Glu-Nmg-Asp-Trp-Leu-Gly-Trp-Gln- | 1614.664 | 1614.671 | 7.9 | 3BP- | A |
| 3307 | Cys]-NH2 | | | | 2776 | |
| 3BP- | Ac-Val-Tyr-[Cys(3MeBn)-Glu-Nmg-Asp-Trp-Leu-ala-Trp-Gln- | 1628.679 | 1628.686 | 8.0 | 3BP- | C |
| 3309 | Cys]-NH2 | | | | 2776 | |
| 3BP- | Ac-Val-Tyr-[Cys(3MeBn)-Glu-Nmg-Asp-Hyw-Leu-Thr-Trp- | 1674.685 | 1674.688 | 7.0 | 3BP- | C |
| 3310 | Gln-Cys]-NH2 | | | | 2776 | |
| 3BP- | Ac-Val-Tyr-[Cys(3MeBn)-Glu-Nmg-Asp-Bta-Leu-Thr-Trp-Gln- | 1675.651 | 1675.655 | 8.4 | 3BP- | A |
| 3311 | Cys]-NH2 | | | | 2776 | |
| 3BP- | Ac-Val-Tyr-[Cys(3MeBn)-Glu-Nmg-Asp-Trp-Leu-Thr-5Clw- | 1692.651 | 1692.657 | 8.4 | 3BP- | C |
| 3312 | Gln-Cys]-NH2 | | | | 2776 | |
| 3BP- | Ac-Val-Tyr-[Cys(3MeBn)-Glu-Nmg-Asp-Trp-Leu-Thr-Trp-Ala- | 1601.668 | 1601.671 | 8.2 | 3BP- | A |
| 3313 | Cys]-NH2 | | | | 2776 | |
| 3BP- | Ac-Val-Tyr-[Cys(3MeBn)-Glu-Nmg-Asp-1Ni-Leu-Thr-Trp-Gln- | 1669.695 | 1669.696 | 8.3 | 3BP- | A |
| 3314 | Cys]-NH2 | | | | 2776 | |
| 3BP- | Ac-Val-Tyr-[Cys(3MeBn)-Glu-Nmg-Asp-Trp-Leu-Thr-Bta-Gln- | 1675.651 | 1675.651 | 8.4 | 3BP- | C |
| 3315 | Cys]-NH2 | | | | 2776 | |
| 3BP- | Ac-[Cys(3MeBn)-Glu-Nmg-Asp-Trp-Leu-Thr-Trp-Gln-Cys]- | 1396.558 | 1396.559 | 7.3 | 3BP- | C |
| 3325 | NH2 | | | | 2776 | |
| 3BP- | InDOTA-Ttds-Val-Tyr-[Cys(3MeBn)-Glu-Gly-Asp-Trp-Leu- | 2402.908 | 2400.913 | 6.6 | 3BP- | B |
| 3328 | Thr-Trp-Gln-Cys]-NH2 | | | | 2776 | |
| 3BP- | Ac-Lys(DOTA)-Val-Tyr-[Cys(3MeBn)-Glu-pro-Asp-Trp-Leu- | 2141.959 | 2141.978 | 6.9 | 3BP- | A |
| 3427 | Thr-Trp-Ala-Cys]-NH2 | | | | 2776 | |
| 3BP- | Ac-Lys(DOTA-O2Oc)-Val-Tyr-[Cys(3MeBn)-Glu-pro-Asp-Trp- | 2287.033 | 2287.037 | 6.9 | 3BP- | A |
| 3428 | Leu-Thr-Trp-Ala-Cys]-NH2 | | | | 2776 | |
| 3BP- | DOTA-Mamb-Val-Tyr-[Cys(3MeBn)-Glu-pro-Asp-Trp-Leu- | 2104.906 | 2104.912 | 7.2 | 3BP- | A |
| 3429 | Thr-Trp-Ala-Cys]-NH2 | | | | 2776 | |
| 3BP- | DOTA-4Amc-Val-Tyr-[Cys(3MeBn)-Glu-pro-Asp-Trp-Leu-Thr- | 2110.953 | 2110.955 | 7.1 | 3BP- | A |
| 3430 | Trp-Ala-Cys]-NH2 | | | | 2776 | |
| 3BP- | DOTA-4Amc-Val-Tyr-[Cys(3MeBn)-Glu-pro-Asp-Trp-Npg- | 2124.969 | 2124.973 | 7.5 | 3BP- | A |
| 3431 | Thr-Trp-Ala-Cys]-NH2 | | | | 2776 | |
| 3BP- | DOTA-APAc-Val-Tyr-[Cys(3MeBn)-Glu-pro-Asp-Trp-Leu-Thr- | 2111.949 | 2111.951 | 6.7 | 3BP- | A |
| 3432 | Trp-Ala-Cys]-NH2 | | | | 2776 | |
| 3BP- | Ac-Val-Tyr-[Cys(3MeBn)-Glu-pro-Asp-Trp-Leu-Thr-Trp-Ala- | 2232.006 | 2232.009 | 7.4 | 3BP- | A |
| 3433 | Cys]-Mamb-Ape-NH | | | | 2776 | |
| 3BP- | Ac-Val-Tyr-[Cys(3MeBn)-Glu-pro-Asp-Trp-Leu-Thr-Trp-Ala- | 2098.953 | 2098.957 | 7.1 | 3BP- | A |
| 3434 | Cys]-Ape-NH-DOTA′ | | | | 2776 | |
| 3BP- | Ac-Val-Tyr-[Cys(3MeBn)-Glu-pro-Asp-Trp-Leu-Thr-Trp-Ala- | 2287.033 | 2287.037 | 6.8 | 3BP- | A |
| 3474 | Cys]-O2Oc-Lys(DOTA)-NH2 | | | | 2776 | |
| 3BP- | Ac-Val-Tyr-[Cys(3MeBn)-Glu-pro-Asp-Trp-Leu-Thr-Trp-Ala- | 2275.012 | 2275.020 | 7.1 | 3BP- | A |
| 3477 | Cys]-Pamb-Lys(DOTA)-NH2 | | | | 2776 | |
| 3BP- | Ac-Val-Tyr-[Cys(3MeBn)-Glu-pro-Asp-Trp-Leu-Thr-Trp- | 2127.943 | 2127.952 | 7.0 | 3BP- | A |
| 3478 | Glu(NH-Apr-DOTA′)-Cys]-NH2 | | | | 2776 | |
| 3BP- | Ac-Val-Tyr-[Cys(3MeBn)-Glu-pro-Asp-Trp-Leu-Thr-Trp- | 2273.017 | 2273.026 | 7.0 | 3BP- | A |
| 3479 | Glu(NH-Apr-O2Oc-DOTA′)-Cys]-NH2 | | | | 2776 | |
| 3BP- | Ac-Val-Tyr-[Cys(3MeBn)-Glu-pro-Asp-Trp-Npg-Thr-Trp-Ala- | 1641.700 | 1641.702 | 8.2 | 3BP | A |
| 3490 | Cys]-NH2 | | | | 2776 | |
| 3BP- | Ac-Val-Tyr-[Cys(3MeBn)-Glu-pro-Asp-Trp-Npg-Thr-Trp-Ser- | 1657.695 | 1657.696 | 8.0 | 3BP- | A |
| 3491 | Cys]-NH2 | | | | 2776 | |
| 3BP- | Ac-Val-Tyr-[Cys(3MeBn)-Glu-pro-Asp-Trp-Leu-Thr-Trp-Ala- | 1655.715 | 1655.718 | 8.8 | 3BP- | A |
| 3492 | Pen]-NH2 | | | | 2776 | |
| 3BP- | Ac-Val-Tyr-[Cys(3MeBn)-Glu-pro-Asp-Trp-Npg-Thr-Trp-Ala- | 1669.731 | 1669.734 | 9.1 | 3BP- | A |
| 3493 | Pen]-NH2 | | | | 2776 | |
| 3BP- | Ac-Val-Tyr-[Cys(3MeBn)-Glu-Nmg-Asp-Trp-Npg-Thr-Trp-Ala- | 1643.715 | 1643.719 | 8.9 | 3BP | A |
| 3501 | Pen]-NH2 | | | | 2776 | |
| 3BP- | Ac-Val-Tyr-[Cys(3MeBn)-Glu-pro-Asp-Trp-Aib-Thr-Trp-Ala- | 1599.653 | 1599.656 | 7.4 | 3BP- | B |
| 3502 | Cys]-NH2 | | | | 2776 | |
| 3BP- | Ac-Val-Tyr-[Cys(3MeBn)-Glu-pro-Asp-Trp-Egz-Thr-Trp-Ala- | 1639.684 | 1639.688 | 8.0 | 3BP- | A |
| 3503 | Cys]-NH2 | | | | 2776 | |
| 3BP- | Ac-Val-Tyr-[Cys(3MeBn)-Glu-Nlys(DOTA)-Asp-Trp-Leu-Thr- | 2044.906 | 2044.910 | 7.0 | 3BP- | A |
| 3562 | Trp-Ala-Cys]-NH2 | | | | 2776 | |
| 3BP- | Ac-Val-Tyr-[Cys(3MeBn)-Glu-pro-Asp-Trp-Leu-Thr-Trp-Ser- | 1643.679 | 1643.684 | 7.7 | 3BP- | A |
| 3565 | Cys]-NH2 | | | | 2776 | |
| 3BP- | Ac-Ser-Tyr-[Cys(3MeBn)-Glu-pro-Asp-Trp-Leu-Thr-Trp-Ser- | 1631.643 | 1631.647 | 7.1 | 3BP- | A |
| 3566 | Cys]-NH2 | | | | 2776 | |
| 3BP- | Ac-Thr-Tyr-[Cys(3MeBn)-Glu-pro-Asp-Trp-Leu-Thr-Trp-Ser- | 1645.658 | 1645.663 | 7.1 | 3BP- | A |
| 3567 | Cys]-NH2 | | | | 2776 | |
| 3BP- | Ac-Ile-Tyr-[Cys(3MeBn)-Glu-pro-Asp-Trp-Leu-Thr-Trp-Ser- | 1657.695 | 1657.699 | 7.9 | 3BP- | A |
| 3568 | Cys]-NH2 | | | | 2776 | |
| 3BP- | Iva-Tyr-[Cys(3MeBn)-Glu-pro-Asp-Trp-Leu-Thr-Trp-Ser- | 1586.657 | 1586.661 | 8.0 | 3BP- | A |
| 3569 | Cys]-NH2 | | | | 2776 | |
| 3BP- | Ac-Val-Phe-[Cys(3MeBn)-Glu-pro-Asp-Trp-Leu-Thr-Trp-Ser- | 1627.684 | 1627.689 | 8.2 | 3BP- | A |
| 3570 | Cys]-NH2 | | | | 2776 | |
| 3BP- | Ac-Val-Ser-[Cys(3MeBn)-Glu-pro-Asp-Trp-Leu-Thr-Trp-Ser- | 1567.648 | 1567.652 | 7.5 | 3BP- | A |
| 3571 | Cys]-NH2 | | | | 2776 | |
| 3BP- | Ac-Val-Tic-[Cys(3MeBn)-Glu-pro-Asp-Trp-Leu-Thr-Trp-Ser- | 1639.684 | 1639.688 | 8.2 | 3BP- | B |
| 3572 | Cys]-NH2 | | | | 2776 | |
| 3BP- | DOTA-APAc-Val-Tyr-[Cys(3MeBn)-Glu-pro-Asp-Trp-Leu-Thr- | 2127.943 | 2127.951 | 6.8 | 3BP- | A |
| 3583 | Trp-Ser-Cys]-NH2 | | | | 2776 | |
| 3BP- | DOTA-APAc-Val-Tyr-[Cys(3MeBn)-Glu-pro-Asp-Trp-Egz-Thr- | 2139.943 | 2139.952 | 6.9 | 3BP- | A |
| 3584 | Trp-Ser-Cys]-NH2 | | | | 2776 | |
| 3BP- | DOTA-APAc-Val-Tyr-[Cys(3MeBn)-Glu-pro-Asp-Trp-Thp-Thr- | 2141.923 | 2141.933 | 6.4 | 3BP | A |
| 3585 | Trp-Ser-Cys]-NH2 | | | | 2776 | |
| 3BP- | DOTA-APAc-Val-Tyr-[Cys(3MeBn)-Glu-pro-Asp-Trp-Aic-Thr- | 2173.928 | 2173.937 | 7.2 | 3BP | A |
| 3586 | Trp-Ser-Cys]-NH2 | | | | 2776 | |
| 3BP- | DOTA-APAc-Val-Tyr-[Cys(3MeBn)-Glu-pro-Asp-Trp-Eca-Thr- | 2125.928 | 2125.934 | 6.8 | 3BP- | A |
| 3587 | Trp-Ser-Cys]-NH2 | | | | 2776 | |
| 3BP- | Ac-Val-Arg-[Cys(3MeBn)-Glu-pro-Asp-Trp-Leu-Thr-Trp-Ser- | 1636.717 | 1636.722 | 7.3 | 3BP | A |
| 3588 | Cys]-NH2 | | | | 2776 | |
| 3BP- | Ac-Val-Nmy-[Cys(3MeBn)-Glu-pro-Asp-Trp-Leu-Thr-Trp-Ser- | 1657.695 | 1657.699 | 7.6 | 3BP- | A |
| 3589 | Cys]-NH2 | | | | 2776 | |
| 3BP- | DOTA-APAc-Val-Tyr-[Cys(3MeBn)-Glu-pro-Ser-Trp-Leu-Thr- | 2099.949 | 2099.953 | 6.9 | 3BP- | A |
| 3595 | Trp-Ser-Cys]-NH2 | | | | 2776 | |
| 3BP- | DOTA-APAc-Val-Tyr-[Cys(3MeBn)-Glu-pro-Asp-Phe-Leu- | 2088.933 | 2088.937 | 6.9 | 3BP- | A |
| 3596 | Thr-Trp-Ser-Cys]-NH2 | | | | 2776 | |
| 3BP- | DOTA-APAc-Val-Tyr-[Cys(3MeBn)-Glu-pro-Asp-Trp-Leu-His- | 2163.955 | 2163.958 | 6.3 | 3BP- | A |
| 3597 | Trp-Ser-Cys]-NH2 | | | | 2776 | |
| 3BP- | DOTA-APAc-Val-Tyr-[Cys(3MeBn)-Glu-pro-Asp-Trp-Leu-Thr- | 2197.013 | 2197.020 | 6.6 | 3BP- | A |
| 3599 | Trp-Arg-Cys]-NH2 | | | | 2776 | |
| 3BP- | Ac-Val-Tyr-[cys(3mebn)-Bal-Trp-Leu-Thr-Trp-Ser-Cys]- | 1373.594 | 1373.599 | 7.9 | 3BP | C |
| 3604 | NH2 | | | | 2776 | |
| 3BP- | Iva-Asp-[Cys(3MeBn)-Glu-pro-Asp-Trp-Leu-Thr-Trp-Ser- | 1538.621 | 1538.625 | 7.8 | 3BP- | A |
| 3727 | Cys]-NH2 | | | | 2776 | |
| 3BP- | Iva-Glu-[Cys(3MeBn)-Glu-pro-Asp-Trp-Leu-Thr-Trp-Ser- | 1552.637 | 1552.641 | 7.7 | 3BP- | A |
| 3728 | Cys]-NH2 | | | | 2776 | |
| 3BP- | Ac-Tyr-[Cys(3MeBn)-Glu-pro-Asp-Trp-Leu-Thr-Trp-Ser- | 1544.611 | 1544.615 | 7.3 | 3BP- | A |
| 3729 | Cys]-NH2 | | | | 2776 | |
| 3BP- | 3OHPr-Tyr-[Cys(3MeBn)-Glu-pro-Asp-Trp-Leu-Thr-Trp-Ser- | 1574.621 | 1574.625 | 7.1 | 3BP | A |
| 3730 | Cys]-NH2 | | | | 2776 | |
| 3BP- | 4OHPhp-[Cys(3MeBn)-Glu-pro-Asp-Trp-Leu-Thr-Trp-Ser- | 1487.589 | 1487.592 | 7.5 | 3BP- | B |
| 3731 | Cys]-NH2 | | | | 2776 | |
| 3BP- | Ac-Ser-Tyr-[Cys(3MeBn)-Glu-pro-Asp-Trp-Leu-Thr-Trp- | 2260.981 | 2260.980 | 6.4 | 3BP- | A |
| 3732 | Glu(NH-Apr-O2Oc-DOTA′)-Cys]-NH2 | | | | 2776 | |
| 3BP- | Ac-Val-Phe-[Cys(3MeBn)-Glu-pro-Asp-Trp-Leu-Thr-Trp- | 2257.022 | 2257.019 | 7.2 | 3BP- | A |
| 3733 | Glu(NH-Apr-O2Oc-DOTA′)-Cys]-NH2 | | | | 2776 | |
| 3BP- | Ac-Val-Arg-[Cys(3MeBn)-Glu-pro-Asp-Trp-Leu-Thr-Trp- | 2266.055 | 2266.054 | 6.5 | 3BP- | A |
| 3734 | Glu(NH-Apr-O2Oc-DOTA′)-Cys]-NH2 | | | | 2776 | |
| 3BP- | Ac-Ser-Arg-[Cys(3MeBn)-Glu-pro-Asp-Trp-Leu-Thr-Trp- | 2254.019 | 2254.018 | 6.0 | 3BP | A |
| 3735 | Glu(NH-Apr-O2Oc-DOTA′)-Cys]-NH2 | | | | 2776 | |
| 3BP- | Iva-Tyr-[Cys(3MeBn)-Glu-pro-Asp-Trp-Leu-Thr-Trp- | 2215.996 | 2216.004 | 7.1 | 3BP- | A |
| 3740 | Glu(NH-Apr-O2Oc-DOTA′)-Cys]-NH2 | | | | 2776 | |
| 3BP- | Iva-Arg-[Cys(3MeBn)-Glu-pro-Asp-Trp-Leu-Thr-Trp- | 2209.034 | 2209.047 | 6.6 | 3BP | B |
| 3741 | Glu(NH-Apr-O2Oc-DOTA′)-Cys]-NH2 | | | | 2776 | |
| 3BP- | Iva-Phe-[Cys(3MeBn)-Glu-pro-Asp-Trp-Leu-Thr-Trp- | 2200.001 | 2200.011 | 7.7 | 3BP- | A |
| 3742 | Glu(NH-Apr-O2Oc-DOTA′)-Cys]-NH2 | | | | 2776 | |
| 3BP- | Iva-Ser-[Cys(3MeBn)-Glu-pro-Asp-Trp-Leu-Thr-Trp- | 2139.965 | 2139.973 | 6.9 | 3BP- | A |
| 3743 | Glu(NH-Apr-O2Oc-DOTA′)-Cys]-NH2 | | | | 2776 | |
| 3BP- | EuDOTA-APAc-Val-Tyr-[Cys(3MeBn)-Glu-pro-Asp-Trp-Leu- | 2277.841 | 2275.843 | 6.8 | 3BP- | A |
| 3784 | Thr-Trp-Ser-Cys]-NH2 | | | | 2776 | |
| 3BP- | Ac-Lys(DOTA)-Val-Tyr-[Cys(3MeBn)-Glu-pro-Asp-Trp-Leu- | 2157.954 | \|2157.980 | 6.9 | 3BP- | A |
| 3840 | Thr-Trp-Ser-Cys]-NH2 | | | | 2776 | |
| 3BP- | Ac-Tyr-[Cys(3MeBn)-Glu-pro-Asp-Trp-Leu-Thr-Trp-Glu(NH- | 2028.875 | 2028.896 | 6.6 | 3BP- | A |
| 3841 | Apr-DOTA′)-Cys]-NH2 | | | | 2776 | |
| 3BP- | 3OHPr-Tyr-[Cys(3MeBn)-Glu-pro-Asp-Trp-Leu-Thr-Trp- | 2058.886 | 2058.912 | 6.5 | 3BP- | A |
| 3842 | Glu(NH-Apr-DOTA′)-Cys]-NH2 | | | | 2776 | |
| 3BP- | Ac-Val-Tyr-[Cys(3MeBn)-Glu-pro-Asp-Trp-Leu-Thr-Trp- | 2384.898 | 2382.908 | 6.9 | 3BP- | A |
| 3843 | Glu(NH-Apr-O2Oc-InDOTA′)-Cys]-NH2 | | | | 2776 | |
| 3BP- | Ac-Val-Phe-[Cys(3MeBn)-Glu-pro-Asp-Trp-Leu-Thr-Trp- | 2368.903 | 2366.914 | 7.2 | 3BP- | A |
| 3844 | Glu(NH-Apr-O2Oc-InDOTA′)-Cys]-NH2 | | | | 2776 | |
| 3BP- | Ac-Val-Arg-[Cys(3MeBn)-Glu-pro-Asp-Trp-Leu-Thr-Trp- | 2377.936 | 2375.946 | 6.6 | 3BP- | A |
| 3845 | Glu(NH-Apr-O2Oc-InDOTA′)-Cys]-NH2 | | | | 2776 | |
| 3BP- | DOTA-APAc-[Cys(3MeBn)-Glu-pro-Asp-Trp-Leu-Thr-Trp- | 1865.812 | 1865.840 | 6.6 | 3BP- | A |
| 3858 | Ser-Cys]-NH2 | | | | 2776 | |
| 3BP- | DOTA-Tyr-[Cys(3MeBn)-Glu-pro-Asp-Trp-Leu-Thr-Trp-Ser- | 1888.780 | 1888.807 | 6.5 | 3BP- | A |
| 3859 | Cys]-NH2 | | | | 2776 | |
| 3BP- | DOTA-APAc-Tyr-[Cys(3MeBn)-Glu-pro-Asp-Trp-Leu-Thr-Trp- | 2028.875 | 2028.885 | 6.4 | 3BP- | A |
| 3867 | Ser-Cys]-NH2 | | | | 2776 | |
| 3BP- | DOTA-4Amc-Tyr-[Cys(3MeBn)-Glu-pro-Asp-Trp-Leu-Thr- | 2027.880 | 2027.890 | 6.7 | 3BP- | A |
| 3868 | Trp-Ser-Cys]-NH2 | | | | 2776 | |
| 3BP- | DOTA-O2Oc-Tyr-[Cys(3MeBn)-Glu-pro-Asp-Trp-Leu-Thr- | 2033.854 | 2033.862 | 6.5 | 3BP- | A |
| 3869 | Trp-Ser-Cys]-NH2 | | | | 2776 | |
| 3BP- | DOTA-Ttds-Nle-Tyr-[Cys(3MeBn)-Glu-pro-Asp-Trp-Leu-Thr- | 2304.048 | 2304.059 | 7.2 | 3BP- | A |
| 3870 | Trp-Ser-Cys]-NH2 | | | | 2776 | |
| 3BP- | Iva-Asp-[Cys(3MeBn)-Glu-pro-Asp-Trp-Leu-Thr-Trp- | 2167.960 | 2167.972 | 6.9 | 3BP- | B |
| 3871 | Glu(NH-Apr-O2Oc-DOTA′)-Cys]-NH2 | | | | 2776 | |
| 3BP- | Iva-Glu-[Cys(3MeBn)-Glu-pro-Asp-Trp-Leu-Thr-Trp- | 2181.975 | 2181.987 | 6.8 | 3BP- | B |
| 3872 | Glu(NH-Apr-O2Oc-DOTA′)-Cys]-NH2 | | | | 2776 | |
| 3BP- | Ac-Ser-Tyr-[Cys(3MeBn)-Glu-pro-Asp-Trp-Leu-Thr-Trp- | 2115.907 | 2115.920 | 6.5 | 3BP- | A |
| 3873 | Glu(NH-Apr-DOTA′)-Cys]-NH2 | | | | 2776 | |
| 3BP- | Ac-Val-Phe-[Cys(3MeBn)-Glu-pro-Asp-Trp-Leu-Thr-Trp- | 2111.949 | 2111.959 | 7.3 | 3BP- | A |
| 3874 | Glu(NH-Apr-DOTA′)-Cys]-NH2 | | | | 2776 | |
| 3BP- | Ac-Val-Tyr-[Cys(3MeBn)-{Lys-pro-Asp-Trp-Leu-Glu}- | 1652.716 | 1652.725 | 7.8 | 3BP- | B |
| 3888 | Trp-Ser-Cys]-NH2 | | | | 2776 | |
| 3BP- | Ac-Val-Tyr-[Cys(3MeBn)-Glu-pro-{Lys-Trp-Leu-Glu}- | 1666.731 | 1666.743 | 7.5 | 3BP- | A |
| 3890 | Trp-Ser-Cys]-NH2 | | | | 2776 | |
| 3BP- | Ac-Val-Tyr-[Cys(3MeBn)-{Glu-pro-Asp-Trp-Leu-Lys}- | 1652.716 | 1652.728 | 7.9 | 3BP- | C |
| 3899 | Trp-Ser-Cys]-NH2 | | | | 2776 | |
| 3BP- | Ac-Val-Tyr-[Cys(3MeBn)-Glu-pro-{Glu-Trp-Leu-Lys}- | 1666.731 | 1666.744 | 7.5 | 3BP- | B |
| 3901 | Trp-Ser-Cys]-NH2 | | | | 2776 | |
| 3BP- | InDOTA-APAc-Val-Tyr-[Cys(3MeBn)-Glu-pro-Asp-Trp-Leu- | 2239.824 | 2237.833 | 6.8 | 3BP- | A |
| 3905 | Thr-Trp-Ser-Cys]-NH2 | | | | 2776 | |
| 3BP- | InDOTA-Tyr-[Cys(3MeBn)-Glu-pro-Asp-Trp-Leu-Thr-Trp- | 2000.660 | 1998.666 | 6.6 | 3BP- | A |
| 3906 | Ser-Cys]-NH2 | | | | 2776 | |
| 3BP- | DOTA-APAc-Val-Tyr-[Cys(3MeBn)-Glu-pro-{Lys-Trp-Leu- | 2150.996 | 2150.999 | 6.5 | 3BP- | B |
| 3933 | Glu}-Trp-Ser-Cys]-NH2 | | | | 2776 | |
| 3BP- | DOTA-Tyr-[Cys(3MeBn)-Glu-pro-{Lys-Trp-Leu-Glu}-Trp- | 1911.832 | 1911.835 | 6.6 | 3BP- | A |
| 3934 | Ser-Cys]-NH2 | | | | 2776 | |
| 3BP- | Ac-Val-Tyr-[Cys(3MeBn)-Glu-pro-{Lys-Trp-Leu-glu}- | 1666.731 | 1666.739 | 8.0 | 3BP- | A |
| 3957 | Trp-Ser-Cys]-NH2 | | | | 2776 | |
| 3BP- | Ac-Val-Tyr-[Cys(3MeBn)-Glu-pro-{Orn-Trp-Leu-Glu}- | 1652.716 | 1652.723 | 7.9 | 3BP- | B |
| 3958 | Trp-Ser-Cys]-NH2 | | | | 2776 | |
| 3BP- | Ac-Val-Tyr-[Cys(3MeBn)-Glu-pro-{Dab-Trp-Leu-Glu}-Trp- | 1638.700 | 1638.717 | 8.1 | 3BP- | B |
| 3960 | Ser-Cys]-NH2 | | | | 2776 | |
| 3BP- | Ac-Val-Tyr-[Cys(3MeBn)-Glu-pro-{Lys-Trp-Egz-Glu}- | 1678.731 | 1678.748 | 8.0 | 3BP- | B |
| 3961 | Trp-Ser-Cys]-NH2 | | | | 2776 | |
| 3BP- | Ac-Val-Tyr-[Cys(3MeBn)-Glu-pro-{Lys-Trp-Thp-Glu}- | 1680.711 | 1680.718 | 7.2 | 3BP- | C |
| 3962 | Trp-Ser-Cys]-NH2 | | | | 2776 | |
| 3BP- | Ac-Val-Tyr-[Cys(3MeBn)-Glu-pro-{Lys-Trp-Leu-Glu}- | 1694.763 | 1694.781 | 8.4 | 3BP- | A |
| 3963 | Trp-Ser-Pen]-NH2 | | | | 2776 | |
| 3BP- | Ac-Val-Tyr-[Cys(3MeBn)-Glu-pro-{Orn-Trp-Leu-Asp}-Trp- | 1638.700 | 1638.705 | 8.2 | 3BP- | A |
| 4005 | Ser-Cys]-NH2 | | | | 2776 | |
| 3BP- | Ac-Val-Tyr-[Cys(3MeBn)-Glu-pro-{Lys-Trp-Leu-asp}- | 1652.716 | 1652.732 | 8.4 | 3BP- | C |
| 4043 | Trp-Ser-Cys]-NH2 | | | | 2776 | |
| 3BP- | Ac-Val-Tyr-[Cys(3MeBn)-Glu-pro-{Lys-Trp-Leu-Asp}-Trp- | 1652.716 | 1652.722 | 8.1 | 3BP- | B |
| 4046 | Ser-Cys]-NH2 | | | | 2776 | |
| 3BP- | Ac-Val-Tyr-[Cys(3MeBn)-Glu-pro-{Dab-Trp-Leu-Asp}-Trp- | 1624.684 | 1624.689 | 8.2 | 3BP- | C |
| 4050 | Ser-Cys]-NH2 | | | | 2776 | |
| 3BP- | InDOTA-APAc-Val-Tyr-[Cys(3MeBn)-Glu-pro-{Lys-Trp-Leu- | 2262.876 | 2260.886 | 7.0 | 3BP- | B |
| 4052 | Glu}-Trp-Ser-Cys]-NH2 | | | | 2776 | |
| 3BP- | InDOTA-Tyr-[Cys(3MeBn)-Glu-pro-{Lys-Trp-Leu-Glu}-Trp- | 2023.713 | 2021.721 | 6.9 | 3BP- | B |
| 4053 | Ser-Cys]-NH2 | | | | 2776 | |
| 3BP- | DOTA-APAc-Val-Dmo-[Cys(3MeBn)-Glu-pro-Asp-Trp-Leu- | 2106.991 | 2107.001 | 6.8 | 3BP- | A |
| 4095 | Thr-Trp-Ser-Cys]-NH2 | | | | 2776 | |
| 3BP- | DOTA-APAc-Val-Tyr-[Cys(3MeBn)-Dmo-pro-Asp-Trp-Leu- | 2141.011 | 2141.023 | 6.7 | 3BP- | A |
| 4096 | Thr-Trp-Ser-Cys]-NH2 | | | | 2776 | |
| 3BP- | DOTA-APAc-Val-Tyr-[Cys(3MeBn)-Glu-dmo-Asp-Trp-Leu- | 2173.001 | 2173.013 | 6.6 | 3BP- | A |
| 4097 | Thr-Trp-Ser-Cys]-NH2 | | | | 2776 | |
| 3BP- | DOTA-APAc-Val-Tyr-[Cys(3MeBn)-Glu-pro-Dmo-Trp-Leu- | 2155.027 | 2155.038 | 5.9 | 3BP- | B |
| 4098 | Thr-Trp-Ser-Cys]-NH2 | | | | 2776 | |
| 3BP- | DOTA-APAc-Val-Tyr-[Cys(3MeBn)-Glu-pro-Asp-Trp-Leu- | 2169.006 | 2169.018 | 6.6 | 3BP- | A |
| 4099 | Dmo-Trp-Ser-Cys]-NH2 | | | | 2776 | |
| 3BP- | DOTA-APAc-Val-Tyr-[Cys(3MeBn)-Glu-pro-Asp-Trp-Leu-Thr- | 2183.022 | 2183.035 | 6.8 | 3BP- | A |
| 4100 | Trp-Dmo-Cys]-NH2 | | | | 2776 | |
| 3BP- | DOTA-APAc-Val-Tyr-[Cys(3MeBn)-Hse-pro-Asp-Trp-Leu- | 2099.949 | 2099.968 | 7.0 | 3BP- | A |
| 4101 | Thr-Trp-Ser-Cys]-NH2 | | | | 2776 | |
| 3BP- | DOTA-APAc-Val-Tyr-[Cys(3MeBn)-Glu-pro-Hse-Trp-Leu-Thr- | 2113.964 | 2113.986 | 7.0 | 3BP- | A |
| 4102 | Trp-Ser-Cys]-NH2 | | | | 2776 | |
| 3BP- | DOTA-APAc-Val-Tyr-[Cys(3MeBn)-Glu-pro-Asn-Trp-Leu-Thr- | 2126.959 | 2126.965 | 7.0 | 3BP- | A |
| 4103 | Trp-Ser-Cys]-NH2 | | | | 2776 | |
| 3BP- | DOTA-APAc-Val-Tyr-[Cys(3MeBn)-Gln-pro-Asp-Trp-Leu-Thr- | 2126.959 | 2126.981 | 7.0 | 3BP- | A |
| 4104 | Trp-Ser-Cys]-NH2 | | | | 2776 | |
| 3BP- | DOTA-APAc-Val-Ser-[Cys(3MeBn)-Glu-pro-Asp-Trp-Leu- | 2051.912 | 2051.921 | 7.2 | 3BP- | A |
| 4105 | Thr-Trp-Ser-Cys]-NH2 | | | | 2776 | |
| 3BP- | DOTA-APAc-Val-Tyr-[Cys(3MeBn)-Glu-pro-Asp-Trp-Aml-Thr- | 2141.959 | 2141.965 | 7.3 | 3BP- | B |
| 4106 | Trp-Ser-Cys]-NH2 | | | | 2776 | |
| 3BP- | DOTA-APAc-Val-Tyr-[Cys(3MeBn)-Glu-pro-Nmd-Trp-Leu- | 2141.959 | 2141.980 | 7.1 | 3BP- | A |
| 4107 | Thr-Trp-Ser-Cys]-NH2 | | | | 2776 | |
| 3BP- | DOTA-APAc-Val-Tyr-[Cys(3MeBn)-Nms-pro-Asp-Trp-Leu- | 2099.949 | 2099.957 | 6.7 | 3BP- | A |
| 4108 | Thr-Trp-Ser-Cys]-NH2 | | | | 2776 | |
| 3BP- | DOTA-APAc-Val-Glu(AGLU′)-[Cys(3MeBn)-Glu-pro-Asp-Trp- | 2257.007 | 2257.023 | 6.9 | 3BP- | A |
| 4112 | Leu-Thr-Trp-Ser-Cys]-NH2 | | | | 2776 | |
| 3BP- | DOTA-APAc-Val-Tyr-[Cys(3MeBn)-Glu(AGLU′)-pro-Asp-Trp- | 2291.028 | 2291.043 | 6.8 | 3BP- | A |
| 4113 | Leu-Thr-Trp-Ser-Cys]-NH2 | | | | 2776 | |
| 3BP- | DOTA-APAc-Val-Tyr-[Cys(3MeBn)-Glu-{lys-Asp-Trp-Leu- | 2168.970 | 2168.980 | 7.2 | 3BP- | B |
| 4114 | Glu}-Trp-Ser-Cys]-NH2 | | | | 2776 | |
| 3BP- | DOTA-APAc-Val-Tyr-[Cys(3MeBn)-Glu-{lys-Ser-Trp-Leu- | 2140.975 | 2140.987 | 7.2 | 3BP- | B |
| 4115 | Glu}-Trp-Ser-Cys]-NH2 | | | | 2776 | |
| 3BP- | DOTA-APAc-Val-Tyr-[Cys(3MeBn)-Glu-glu(AGLU′)-Asp-Trp- | 2323.018 | 2323.034 | 6.5 | 3BP- | A |
| 4119 | Leu-Thr-Trp-Ser-Cys]-NH2 | | | | 2776 | |
| 3BP- | DOTA-APAc-Val-Tyr-[Cys(3MeBn)-Glu-pro-Glu(AGLU′)-Trp- | 2305.044 | 2305.060 | 6.2 | 3BP- | B |
| 4120 | Leu-Thr-Trp-Ser-Cys]-NH2 | | | | 2776 | |
| 3BP- | DOTA-APAc-Val-Tyr-[Cys(3MeBn)-Glu-pro-Asp-Trp-Leu- | 2319.023 | 2319.042 | 6.9 | 3BP- | A |
| 4121 | Glu(AGLU′)-Trp-Ser-Cys]-NH2 | | | | 2776 | |
| 3BP- | DOTA-APAc-Val-Tyr-[Cys(3MeBn)-Glu-pro-Asp-Trp-Leu-Thr- | 2333.038 | 2333.059 | 6.9 | 3BP- | A |
| 4122 | Trp-Glu(AGLU′)-Cys]-NH2 | | | | 2776 | |
| 3BP- | DOTA-APAc-Val-Tyr-[Cys(3MeBn)-Glu-pro-Eem-Trp-Leu- | 2237.962 | 2237.980 | 8.1 | 3BP | A |
| 4123 | Thr-Trp-Ser-Cys]-NH2 | | | | 2776 | |
| 3BP- | DOTA-APAc-Val-Tyr-[Cys(3MeBn)-Glu-pro-Tyr(Bzl)-Trp-Leu- | 2266.027 | 2266.035 | 8.5 | 3BP- | A |
| 4127 | Thr-Trp-Ser-Cys]-NH2 | | | | 2776 | |
| 3BP- | DOTA-APAc-Val-Tyr-[Cys(3MeBn)-Glu-pro-Cys(Bzl)-Trp- | 2205.973 | 2205.988 | 8.8 | 3BP | A |
| 4128 | Leu-Thr-Trp-Ser-Cys]-NH2 | | | | 2776 | |
| 3BP- | DOTA-APAc-Val-Tyr-[Cys(3MeBn)-Glu-pro-Cys(2Quyl)-Trp- | 2256.984 | 2257.001 | 7.4 | 3BP- | A |
| 4129 | Leu-Thr-Trp-Ser-Cys]-NH2 | | | | 2776 | |
| 3BP- | DOTA-APAc-Val-Tyr-[Cys(3MeBn)-Glu-pro-Asp-Cys(Bzl)- | 2134.920 | 2134.938 | 7.6 | 3BP- | A |
| 4130 | Leu-Thr-Trp-Ser-Cys]-NH2 | | | | 2776 | |
| 3BP- | DOTA-APAc-Val-Tyr-[Cys(3MeBn)-Glu-pro-Asp-Bip-Leu-Thr- | 2164.964 | 2164.980 | 8.3 | 3BP- | A |
| 4131 | Trp-Ser-Cys]-NH2 | | | | 2776 | |
| 3BP- | DOTA-APAc-Val-Tyr-[Cys(3MeBn)-Glu-pro-Asp-Dip-Leu-Thr- | 2164.964 | 2164.976 | 8.1 | 3BP- | A |
| 4132 | Trp-Ser-Cys]-NH2 | | | | 2776 | |
| 3BP- | DOTA-APAc-Val-Tyr-[Cys(3MeBn)-Glu-pro-Asp-Eaa-Leu- | 2156.855 | 2156.863 | 8.0 | 3BP- | A |
| 4133 | Thr-Trp-Ser-Cys]-NH2 | | | | 2776 | |
| 3BP- | DOTA-APAc-Val-Tyr-[Cys(3MeBn)-Glu-pro-Asp-Pif-Leu-Thr- | 2214.829 | 2214.839 | 8.0 | 3BP- | A |
| 4134 | Trp-Ser-Cys]-NH2 | | | | 2776 | |
| 3BP- | DOTA-APAc-Val-Tyr-[Cys(3MeBn)-Glu-pro-Asp-Mtf-Leu-Thr- | 2156.920 | 2156.929 | 7.9 | 3BP- | A |
| 4135 | Trp-Ser-Cys]-NH2 | | | | 2776 | |
| 3BP- | DOTA-APAc-Val-Tyr-[Cys(3MeBn)-Glu-pro-Asp-Tyr-Leu-Thr- | 2104.927 | 2104.946 | 6.3 | 3BP- | A |
| 4136 | Trp-Ser-Cys]-NH2 | | | | 2776 | |
| 3BP- | DOTA-APAc-Val-Tyr-[Cys(3MeBn)-Glu-pro-Asp-Tyr(Bzl)- | 2194.974 | 2194.995 | 8.4 | 3BP- | B |
| 4137 | Leu-Thr-Trp-Ser-Cys]-NH2 | | | | 2776 | |
| 3BP- | DOTA-APAc-Val-Tyr-[Cys(3MeBn)-Glu-pro-Asp-Ptf-Leu-Thr- | 2156.920 | 2156.930 | 8.0 | 3BP- | A |
| 4139 | Trp-Ser-Cys]-NH2 | | | | 2776 | |
| 3BP- | DOTA-APAc-Val-Tyr-[Cys(2Lut)-Glu-pro-Asp-Trp-Leu-Thr- | 2128.939 | 2128.944 | 6.0 | 3BP- | B |
| 4140 | Trp-Ser-Cys]-NH2 | | | | 2776 | |
| 3BP- | DOTA-APAc-Val-Tyr-[Cys(3Lut)-Glu-pro-Asp-Trp-Leu-Thr- | 2128.939 | 2128.945 | 5.9 | 3BP- | C |
| 4141 | Trp-Ser-Cys]-NH2 | | | | 2776 | |
| 3BP- | DOTA-APAc-Val-Tyr-[Cys(3MeBn)-Glu-pro-Asp-Trp-Npg- | 2141.959 | 2141.979 | 7.4 | 3BP- | A |
| 4142 | Thr-Trp-Ser-Cys]-NH2 | | | | 2776 | |
| 3BP- | DOTA-APAc-Val-Tyr-[Cys(3MeBn)-Glu-pro-Asp-Trp-Cha- | 2167.975 | 2167.994 | 7.7 | 3BP- | A |
| 4143 | Thr-Trp-Ser-Cys]-NH2 | | | | 2776 | |
| 3BP- | DOTA-APAc-Val-Tyr-[Cys(3MeBn)-Glu-pro-Asp-1MW-Leu- | 2141.959 | 2141.979 | 7.3 | 3BP- | A |
| 4144 | Thr-Trp-Ser-Cys]-NH2 | | | | 2776 | |
| 3BP- | DOTA-APAc-Val-Tyr-[Cys(3MeBn)-Glu-pro-Asp-7MW-Leu- | 2141.959 | 2141.971 | 7.1 | 3BP- | A |
| 4158 | Thr-Trp-Ser-Cys]-NH2 | | | | 2776 | |
| 3BP- | DOTA-APAc-Val-Tyr-[Cys(3MeBn)-Glu-pro-Asp-Trp-Leu-Thr- | 2128.939 | 2128.953 | 5.8 | 3BP- | A |
| 4159 | 7Nw-Ser-Cys]-NH2 | | | | 2776 | |
| 3BP- | DOTA-APAc-Val-Tyr-[Cys(3MeBn)-Glu-pro-Asp-Eap-Leu- | 2144.995 | 2145.010 | 8.4 | 3BP- | B |
| 4160 | Thr-Trp-Ser-Cys]-NH2 | | | | 2776 | |
| 3BP- | DOTA-APAc-Val-Tyr-[Cys(3MeBn)-Glu-pro-Asp-Aph(Bz)- | 2207.970 | 2207.984 | 7.5 | 3BP- | C |
| 4171 | Leu-Thr-Trp-Ser-Cys]-NH2 | | | | 2776 | |
| 3BP- | DOTA-APAc-Val-Tyr-[Cys(3MeBn)-Glu-pro-Asp-Aph(Cp)- | 2200.001 | 2200.016 | 7.6 | 3BP- | B |
| 4173 | Leu-Thr-Trp-Ser-Cys]-NH2 | | | | 2776 | |
| 3BP- | DOTA-APAc-Val-{Asp-[Cys(3MeBn)-Glu-pro-Asp-Trp-Leu- | 2060.913 | 2060.918 | 7.6 | 3BP- | A |
| 4174 | Thr-Trp-Dap}-Cys]-NH2 | | | | 2776 | |
| 3BP- | DOTA-APAc-Val-{Glu-[Cys(3MeBn)-Glu-pro-Asp-Trp-Leu- | 2074.928 | 2074.932 | 7.0 | 3BP- | A |
| 4175 | Thr-Trp-Dap}-Cys]-NH2 | | | | 2776 | |
| 3BP- | DOTA-APAc-Val-{Dap-[Cys(3MeBn)-Glu-pro-Asp-Trp-Leu- | 2060.913 | 2060.917 | 8.0 | 3BP- | A |
| 4176 | Thr-Trp-Asp}-Cys]-NH2 | | | | 2776 | |
| 3BP- | DOTA-APAc-Val-{Dap-[Cys(3MeBn)-Glu-pro-Asp-Trp-Leu- | 2074.928 | 2074.933 | 7.3 | 3BP- | A |
| 4177 | Thr-Trp-Glu}-Cys]-NH2 | | | | 2776 | |
| 3BP- | [pro-Pro-Val-Tyr-{Cys(3MeBn)-Glu-pro-Asp-Trp-Leu- | 1778.747 | 1778.751 | 9.2 | 3BP- | A |
| 4178 | Thr-Trp-Ser-Cys}] | | | | 2776 | |
| 3BP- | DOTA-APAc-Val-{Cys-[Cys(3MeBn)-Glu-pro-Asp-Trp-Leu- | 2081.851 | 2081.859 | 7.4 | 3BP- | A |
| 4179 | Thr-Trp-Cys}-Cys]-NH2 | | | | 2776 | |
| 3BP- | DOTA-APAc-Val-Tyr-[Cys(3MeBn)-Glu-pro-Asp-Aph(2Thz)- | 2214.921 | 2214.932 | 7.3 | 3BP- | C |
| 4180 | Leu-Thr-Trp-Ser-Cys]-NH2 | | | | 2776 | |
| 3BP- | DOTA-APAc-Val-Tyr-[Cys(3MeBn)-Glu-pro-Asp-Aph(iNic)- | 2208.965 | 2208.975 | 6.1 | 3BP- | C |
| 4183 | Leu-Thr-Trp-Ser-Cys]-NH2 | | | | 2776 | |
| 3BP- | DOTA-APAc-Val-Tyr-[Cys(3MeBn)-Glu-pro-Glu(NHMe2Nph)- | 2281.038 | 2281.053 | 7.9 | 3BP- | A |
| 4215 | Trp-Leu-Thr-Trp-Ser-Cys]-NH2 | | | | 2776 | |
| 3BP- | DOTA-APAc-Val-Tyr-[Cys(3MeBn)-Glu-pro-Asp- | 2185.985 | 2185.992 | 7.0 | 3BP- | A |
| 4234 | Af3(MCprAc)-Leu-Thr-Trp-Ser-Cys]-NH2 | | | | 2776 | |
| 3BP- | DOTA-APAc-Val-Tyr-[Cys(3MeBn)-Glu-pro-Asp-Af3(Prp)- | 2159.970 | 2159.979 | 6.6 | 3BP- | A |
| 4235 | Leu-Thr-Trp-Ser-Cys]-NH2 | | | | 2776 | |
| 3BP- | DOTA-APAc-Val-Tyr-[Cys(3MeBn)-Glu-pro-Asp-Af3(Iva)- | 2188.001 | 2188.008 | 7.2 | 3BP- | A |
| 4236 | Leu-Thr-Trp-Ser-Cys]-NH2 | | | | 2776 | |
| 3BP- | DOTA-APAc-Val-Tyr-[Cys(3MeBn)-Glu-pro-Asp-Af3(HO- | 2203.960 | 2203.967 | 6.2 | 3BP- | A |
| 4237 | Succinyl)-Leu-Thr-Trp-Ser-Cys]-NH2 | | | | 2776 | |
| 3BP- | DOTA-APAc-Val-Tyr-[Cys(3MeBn)-Glu-pro-Asp-Af3(H2N- | 2202.975 | 2202.975 | 6.0 | 3BP- | B |
| 4238 | Succinyl)-Leu-Thr-Trp-Ser-Cys]-NH2 | | | | 2776 | |
| 3BP- | DOTA-APAc-Val-Tyr-[Cys(3MeBn)-Glu-pro-Asp-Af3(Oa5)- | 2198.944 | 2198.944 | 6.4 | 3BP- | B |
| 4239 | Leu-Thr-Trp-Ser-Cys]-NH2 | | | | 2776 | |
| 3BP- | DOTA-APAc-Val-Tyr-[Cys(3MeBn)-Glu-pro-Asp-Af3(Im5)- | 2197.960 | 2197.959 | 5.8 | 3BP- | B |
| 4240 | Leu-Thr-Trp-Ser-Cys]-NH2 | | | | 2776 | |
| 3BP- | DOTA-APAc-Val-Tyr-[Cys(3MeBn)-Glu-pro-Asp-Af3(H-pGlu)- | 2214.975 | 2214.975 | 6.0 | 3BP- | B |
| 4241 | Leu-Thr-Trp-Ser-Cys]-NH2 | | | | 2776 | |
| 3BP- | DOTA-APAc-Val-Tyr-[Cys(3MeBn)-Glu-pro-Asp-Af3(MSAc)- | 2223.932 | 2223.931 | 6.2 | 3BP- | A |
| 4242 | Leu-Thr-Trp-Ser-Cys]-NH2 | | | | 2776 | |
| 3BP- | DOTA-APAc-Val-Tyr-[Cys(3MeBn)-Glu-Gly-{Apg-Trp-Leu- | 2096.949 | 2096.950 | 6.9 | 3BP- | B |
| 4252 | Glu}-Trp-Ser-Cys]-NH2 | | | | 2776 | |
| 3BP- | DOTA-{Asp-[Cys(3MeBn)-Glu-pro-Asp-Trp-Leu-Thr-Trp- | 1821.749 | 1821.751 | 7.0 | 3BP- | A |
| 4253 | Dap}-Cys]-NH2 | | | | 2776 | |
| 3BP- | DOTA-APAc-{Asp-[Cys(3MeBn)-Glu-pro-Asp-Trp-Leu-Thr- | 1961.844 | 1961.846 | 6.7 | 3BP- | A |
| 4254 | Trp-Dap}-Cys]-NH2 | | | | 2776 | |
| 3BP- | DOTA-PPAc-{Asp-[Cys(3MeBn)-Glu-pro-Asp-Trp-Leu-Thr- | 1947.828 | 1947.831 | 6.6 | 3BP- | A |
| 4255 | Trp-Dap}-Cys]-NH2 | | | | 2776 | |
| 3BP- | DOTA-Inp-{Asp-[Cys(3MeBn)-Glu-pro-Asp-Trp-Leu-Thr-Trp- | 1932.818 | 1932.821 | 6.9 | 3BP- | A |
| 4256 | Dap}-Cys]-NH2 | | | | 2776 | |
| 3BP- | DOTA-Ser-{Asp-[Cys(3MeBn)-Glu-pro-Asp-Trp-Leu-Thr-Trp- | 1908.781 | 1908.783 | 6.8 | 3BP- | A |
| 4257 | Dap}-Cys]-NH2 | | | | 2776 | |
| 3BP- | DOTA-4Amc-{Asp-[Cys(3MeBn)-Glu-pro-Asp-Trp-Leu-Thr- | 1960.849 | 1960.851 | 6.9 | 3BP- | A |
| 4258 | Trp-Dap}-Cys]-NH2 | | | | 2776 | |
| 3BP- | DOTA-Cmp-{Asp-[Cys(3MeBn)-Glu-pro-Asp-Trp-Leu-Thr- | 1946.833 | 1946.834 | 6.8 | 3BP- | A |
| 4259 | Trp-Dap}-Cys]-NH2 | | | | 2776 | |
| 3BP- | DOTA-Mamb-{Asp-[Cys(3MeBn)-Glu-pro-Asp-Trp-Leu-Thr- | 1954.802 | 1954.805 | 7.2 | 3BP- | A |
| 4260 | Trp-Dap}-Cys]-NH2 | | | | 2776 | |
| 3BP- | DOTA-Pamb-{Asp-[Cys(3MeBn)-Glu-pro-Asp-Trp-Leu-Thr- | 1954.802 | 1954.804 | 7.0 | 3BP- | A |
| 4261 | Trp-Dap}-Cys]-NH2 | | | | 2776 | |
| 3BP- | DOTA-O2Oc-{Asp-[Cys(3MeBn)-Glu-pro-Asp-Trp-Leu-Thr- | 1966.823 | 1966.824 | 7.4 | 3BP- | B |
| 4262 | Trp-Dap}-Cys]-NH2 | | | | 4149 | |
| 3BP- | DOTA-Gab-{Asp-[Cys(3MeBn)-Glu-pro-Asp-Trp-Leu-Thr-Trp- | 1906.802 | 1906.804 | 7.6 | 3BP- | A |
| 4263 | Dap}-Cys]-NH2 | | | | 4149 | |
| 3BP- | DOTA-Val-{Asp-[Cys(3MeBn)-Glu-pro-Asp-Trp-Leu-Thr-Trp- | 1920.817 | 1920.818 | 8.0 | 3BP- | A |
| 4264 | Dap}-Cys]-NH2 | | | | 4149 | |
| 3BP- | DOTA-APAc-Val-Tyr-[Cys(3MeBn)-Glu-pro-Asp-Af3(DkpAc)- | 2257.981 | 2257.978 | 6.2 | 3BP- | C |
| 4288 | Leu-Thr-Trp-Ser-Cys]-NH2 | | | | 4149 | |
| 3BP- | DOTA-APAc-Val-Tyr-[Cys(3MeBn)-Glu-pro-Asp-Af3(CMPy)- | 2236.971 | 2236.998 | 7.6 | 3BP- | B |
| 4305 | Leu-Thr-Trp-Ser-Cys]-NH2 | | | | 4149 | |
| 3BP- | DOTA-APAc-Val-Tyr-[Cys(3MeBn)-Glu-pro-Asp-Af3(HySuc)- | 2255.966 | 2255.979 | 6.6 | 3BP- | B |
| 4322 | Leu-Thr-Trp-Ser-Cys]-NH2 | | | | 4149 | |
| 3BP- | DOTA-APAc-Val-Tyr-[Cys(3MeBn)-Glu-pro-Asp-Af3(TzPr)- | 2226.987 | 2226.996 | 6.5 | 3BP- | B |
| 4324 | Leu-Thr-Trp-Ser-Cys]-NH2 | | | | 4149 | |
| 3BP- | DOTA-APAc-Val-Tyr-[Cys(3MeBn)-Glu-pro-Glu(N6iQui)-Trp- | 2282.033 | 2282.040 | 6.8 | 3BP- | C |
| 4328 | Leu-Thr-Trp-Ser-Cys]-NH2 | | | | 4149 | |
| 3BP- | DOTA-APAc-Val-Tyr-[Cys(3MeBn)-Glu-pro- | 2309.000 | 2309.015 | 7.5 | 3BP- | B |
| 4333 | Glu(N4BzISO2Me)-Trp-Leu-Thr-Trp-Ser-Cys]-NH2 | | | | 4149 | |
| 3BP- | DOTA-APAc-Val-Tyr-[Cys(3MeBn)-Glu-pro-Glu(N4BzICI)- | 2264.983 | 2265.002 | 8.5 | 3BP- | B |
| 4334 | Trp-Leu-Thr-Trp-Ser-Cys]-NH2 | | | | 4149 | |
| 3BP- | InDOTA-APAc-Val-{Asp-[Cys(3MeBn)-Glu-pro-Asp-Trp-Leu- | 2170.793 | 2170.801 | 7.8 | 3BP- | A |
| 4337 | Thr-Trp-Dap}-Cys]-NH2 | | | | 4149 | |
| 3BP- | InDOTA-{Asp-[Cys(3MeBn)-Glu-pro-Asp-Trp-Leu-Thr-Trp- | 1931.630 | 1931.640 | 7.8 | 3BP- | A |
| 4338 | Dap}-Cys]-NH2 | | | | 4149 | |
| 3BP- | DOTA-APAc-Val-Tyr-[Cys(3MeBn)-Glu-pro-Glu(NOAzOMe)- | 2266.023 | 2266.044 | 7.6 | 3BP- | B |
| 4339 | Trp-Leu-Thr-Trp-Ser-Cys]-NH2 | | | | 4149 | |
| 3BP- | DOTA-APAc-Val-Tyr-[Cys(3MeBn)-Glu-pro-Glu(N4BzICN)- | 2256.017 | 2256.032 | 7.9 | 3BP- | B |
| 4340 | Trp-Leu-Thr-Trp-Ser-Cys]-NH2 | | | | 4149 | |
| 3BP- | DOTA-APAc-Val-Tyr-[Cys(3MeBn)-Glu-pro-Asp-Af3(DImAc)- | 2285.987 | 2286.008 | 6.4 | 3BP- | B |
| 4341 | Leu-Thr-Trp-Ser-Cys]-NH2 | | | | 4149 | |
| 3BP- | DOTA-APAc-Val-Tyr-[Cys(3MeBn)-Glu-pro- | 2274.028 | 2274.042 | 7.3 | 3BP- | B |
| 4345 | Glu(N4BzICONH2)-Trp-Leu-Thr-Trp-Ser-Cys]-NH2 | | | | 4149 | |
| 3BP- | DOTA-APAc-Val-Tyr-[Cys(3MeBn)-Glu-pro-Asp-Aph(Prp)- | 2159.970 | 2159.981 | 7.2 | 3BP- | C |
| 4347 | Leu-Thr-Trp-Ser-Cys]-NH2 | | | | 4149 | |
| 3BP- | DOTA-APAc-Val-Tyr-[Cys(3MeBn)-Glu-pro-Asp-Aph(Iva)- | 2188.001 | 2188.013 | 8.1 | 3BP- | C |
| 4348 | Leu-Thr-Trp-Ser-Cys]-NH2 | | | | 4149 | |
| 3BP- | DOTA-APAc-Val-Tyr-[Cys(3MeBn)-Glu-pro-Asp-Aph(HO- | 2203.959 | 2203.967 | 6.9 | 3BP- | B |
| 4356 | Succinyl)-Leu-Thr-Trp-Ser-Cys]-NH2 | | | | 4149 | |
| 3BP- | DOTA-APAc-Val-Tyr-[Cys(3MeBn)-Glu-pro-Asp- | 2196.965 | 2196.973 | 6.8 | 3BP- | C |
| 4357 | Aph(CCprAc)-Leu-Thr-Trp-Ser-Cys]-NH2 | | | | 4149 | |
| 3BP- | DOTA-APAc-Val-Tyr-[Cys(3MeBn)-Glu-pro-Asp-Aph(FAc)- | 2163.945 | 2163.953 | 6.7 | 3BP- | C |
| 4358 | Leu-Thr-Trp-Ser-Cys]-NH2 | | | | 4149 | |
| 3BP- | DOTA-APAc-Val-Tyr-[Cys(3MeBn)-Glu-pro-Asp-Aph(MSAc)- | 2223.932 | 2223.940 | 6.5 | 3BP- | C |
| 4360 | Leu-Thr-Trp-Ser-Cys]-NH2 | | | | 4149 | |
| 3BP- | DOTA-APAc-Val-Tyr-[Cys(3MeBn)-Glu-pro-Asp-Af3(Ac-Glu)- | 2274.997 | 2275.005 | 6.6 | 3BP- | B |
| 4361 | Leu-Thr-Trp-Ser-Cys]-NH2 | | | | 4149 | |
| 3BP- | DOTA-APAc-Val-Tyr-[Cys(3MeBn)-Glu-pro-Asp-af3(ac-glu)- | 2274.997 | 2275.007 | 6.8 | 3BP- | B |
| 4362 | Leu-Thr-Trp-Ser-Cys]-NH2 | | | | 4149 | |
| 3BP- | DOTA-APAc-Val-Tyr-[Cys(3MeBn)-Glu-pro-Asp-Af3(Ac-Asp)- | 2260.981 | 2260.989 | 6.5 | 3BP- | B |
| 4363 | Leu-Thr-Trp-Ser-Cys]-NH2 | | | | 4149 | |
| 3BP- | DOTA-APAc-Val-Tyr-[Cys(3MeBn)-Glu-pro-Asp-af3(ac-asp)- | 2260.981 | 2260.989 | 6.7 | 3BP | B |
| 4364 | Leu-Thr-Trp-Ser-Cys]-NH2 | | | | 4149 | |
| 3BP- | DOTA-APAc-Val-Tyr-[Cys(3MeBn)-Glu-pro-Asp-Af3(HO- | 2217.975 | 2217.999 | 6.8 | 3BP- | A |
| 4367 | Glutar)-Leu-Thr-Trp-Ser-Cys]-NH2 | | | | 4149 | |
| 3BP- | DOTA-APAc-Val-Tyr-[Cys(3MeBn)-Glu-pro-Asp-Af3(HO- | 2219.954 | 2219.974 | 6.7 | 3BP- | B |
| 4368 | Dga)-Leu-Thr-Trp-Ser-Cys]-NH2 | | | | 4149 | |
| 3BP- | DOTA-APAc-Val-Tyr-[Cys(3MeBn)-Glu-pro-Asp-Af3(Cpsu)- | 2252.958 | 2252.962 | 7.1 | 3BP- | A |
| 4369 | Leu-Thr-Trp-Ser-Cys]-NH2 | | | | 4149 | |
| 3BP- | DOTA-APAc-Val-Tyr-[Cys(3MeBn)-Glu-pro-Asp-Af3(OPyAc)- | 2238.975 | 2238.999 | 6.8 | 3BP | B |
| 4370 | Leu-Thr-Trp-Ser-Cys]-NH2 | | | | 4149 | |
| 3BP- | DOTA-APAc-Val-Tyr-[Cys(3MeBn)-Glu-pro-Asp- | 2243.966 | 2243.976 | 6.4 | 3BP- | B |
| 4371 | Af3(HYDAc)-Leu-Thr-Trp-Ser-Cys]-NH2 | | | | 4149 | |
| 3BP- | DOTA-APAc-Val-{Asp-[Cys(3MeBn)-Glu-pro-Asp-Af3(HO- | 2136.928 | 2136.944 | 7.0 | 3BP- | A |
| 4377 | Succinyl)-Leu-Thr-Trp-Dap}-Cys]-NH2 | | | | 4149 | |
| 3BP- | DOTA-APAc-Val-Tyr-[Cys(3MeBn)-Glu-pro-Glu(N4Inda)-Trp- | 2271.028 | 2271.053 | 7.5 | 3BP- | B |
| 4387 | Leu-Thr-Trp-Ser-Cys]-NH2 | | | | 4149 | |
| 3BP- | DOTA-APAc-Val-Tyr-[Cys(3MeBn)-Glu-pro- | 2315.054 | 2315.070 | 7.6 | 3BP- | B |
| 4388 | Glu(N4AzPhCONH2)-Trp-Leu-Thr-Trp-Ser-Cys]-NH2 | | | | 4149 | |
| 3BP- | DOTA-APAc-Val-Tyr-[Cys(3MeBn)-Glu-pro- | 2343.066 | 2343.088 | 8.2 | 3BP- | B |
| 4389 | Glu(N4DazPhCN)-Trp-Leu-Thr-Trp-Ser-Cys]-NH2 | | | | 4149 | |
| 3BP- | DOTA-APAc-Val-Tyr-[Cys(3MeBn)-Glu-pro- | 2312.043 | 2312.058 | 7.2 | 3BP- | B |
| 4390 | Glu(N6MeQuion)-Trp-Leu-Thr-Trp-Ser-Cys]-NH2 | | | | 4149 | |
| 3BP- | DOTA-APAc-Val-Tyr-[Cys(3MeBn)-Glu-pro-Asp-Aph(4SaBz)- | 2286.942 | 2286.971 | 6.8 | 3BP- | B |
| 4398 | Leu-Thr-Trp-Ser-Cys]-NH2 | | | | 4149 | |
| 3BP- | DOTA-APAc-Val-Tyr-[Cys(3MeBn)-Glu-pro-Asp- | 2300.958 | 2300.980 | 7.1 | 3BP- | B |
| 4399 | Aph(3MSaBz)-Leu-Thr-Trp-Ser-Cys]-NH2 | | | | 4149 | |
| 3BP- | DOTA-APAc-Val-Tyr-[Cys(3MeBn)-Glu-pro-Asp-Aph(SaPr)- | 2238.942 | 2238.972 | 6.6 | 3BP- | A |
| 4400 | Leu-Thr-Trp-Ser-Cys]-NH2 | | | | 4149 | |
| 3BP- | DOTA-APAc-Val-Tyr-[Cys(3MeBn)-Glu-pro-Asp- | 2292.899 | 2292.927 | 6.9 | 3BP- | A |
| 4401 | Aph(3Ta5Sa)-Leu-Thr-Trp-Ser-Cys]-NH2 | | | | 4149 | |
| 3BP- | DOTA-APAc-Val-Tyr-[Cys(3MeBn)-Glu-pro-Asp- | 2292.899 | 2292.922 | 7.3 | 3BP- | A |
| 4402 | Aph(2Ta5Sa)-Leu-Thr-Trp-Ser-Cys]-NH2 | | | | 4149 | |
| 3BP- | DOTA-APAc-Val-Tyr-[Cys(3MeBn)-Glu-pro-Asp-Af3(4SaBz)- | 2286.942 | 2286.972 | 6.9 | 3BP- | A |
| 4405 | Leu-Thr-Trp-Ser-Cys]-NH2 | | | | 4149 | |
| 3BP- | DOTA-APAc-Val-Tyr-[Cys(3MeBn)-Glu-pro-Asp- | 2300.958 | 2300.985 | 7.2 | 3BP- | A |
| 4406 | Af3(3MSaBz)-Leu-Thr-Trp-Ser-Cys]-NH2 | | | | 4149 | |
| 3BP- | DOTA-APAc-Val-Tyr-[Cys(3MeBn)-Glu-pro-Asp-Af3(SaPr)- | 2238.942 | 2238.967 | 6.7 | 3BP- | A |
| 4407 | Leu-Thr-Trp-Ser-Cys]-NH2 | | | | 4149 | |
| 3BP- | DOTA-APAc-Val-Tyr-[Cys(3MeBn)-Glu-pro-Asp-Aph(3SaBz)- | 2286.942 | 2286.965 | 6.9 | 3BP- | A |
| 4408 | Leu-Thr-Trp-Ser-Cys]-NH2 | | | | 4149 | |
| 3BP- | DOTA-APAc-Val-Tyr-[Cys(3MeBn)-Glu-pro-Asp-Af3(3SaBz)- | 2286.942 | 2286.970 | 6.9 | 3BP- | A |
| 4409 | Leu-Thr-Trp-Ser-Cys]-NH2 | | | | 4149 | |
| 3BP- | DOTA-APAc-Val-Tyr-[Cys(3MeBn)-Glu-pro-Asp- | 2292.899 | 2292.943 | 7.1 | 3BP- | A |
| 4412 | Af3(3Ta5Sa)-Leu-Thr-Trp-Ser-Cys]-NH2 | | | | 4149 | |
| 3BP- | DOTA-APAc-Val-Tyr-[Cys(3MeBn)-Glu-pro-Asp- | 2292.899 | 2292.931 | 7.2 | 3BP- | A |
| 4413 | Af3(2Ta5Sa)-Leu-Thr-Trp-Ser-Cys]-NH2 | | | | 4149 | |
| 3BP- | DOTA-APAc-Val-Tyr-[Cys(3MeBn)-Glu-pro-Asp-Af3(Btda)- | 2327.933 | 2327.958 | 6.6 | 3BP- | C |
| 4415 | Leu-Thr-Trp-Ser-Cys]-NH2 | | | | 4149 | |
| 3BP- | DOTA-APAc-Val-{Asp-[Cys(3MeBn)-Glu-pro-Asp-Eaa-Leu- | 2089.824 | 2089.839 | 7.3 | 3BP- | A |
| 4439 | Thr-Trp-Dap}-Cys]-NH2 | | | | 4149 | |
| 3BP- | DOTA-APAc-Val-{Asp-[Cys(3MeBn)-Glu-pro-Asp-Mtf-Leu- | 2089.889 | 2089.905 | 7.3 | 3BP- | A |
| 4440 | Thr-Trp-Dap}-Cys]-NH2 | | | | 4149 | |
| 3BP- | DOTA-APAc-Val-Tyr-[Cys(3MeBn)-Glu-pro-Glu(NH3PhSa)- | 2295.979 | 2296.032 | 7.5 | 3BP- | B |
| 4441 | Trp-Leu-Thr-Trp-Ser-Cys]-NH2 | | | | 4149 | |
| 3BP- | DOTA-APAc-Val-Tyr-[Cys(3MeBn)-Glu-pro-Glu(NH4PhSa)- | 2295.979 | 2296.041 | 7.5 | 3BP- | B |
| 4442 | Trp-Leu-Thr-Trp-Ser-Cys]-NH2 | | | | 4149 | |
| 3BP- | DOTA-APAc-Val-{Asp-[Cys(3MeBn)-Glu-pro-Asp-Af3(Cpsu)- | 2185.927 | 2185.975 | 7.2 | 3BP- | A |
| 4445 | Leu-Thr-Trp-Dap}-Cys]-NH2 | | | | 4149 | |
| 3BP- | DOTA-PPAc-Tyr-[Cys(3MeBn)-Glu-pro-Asp-Trp-Leu-Thr-Trp- | 2014.859 | 2014.914 | 7.3 | 3BP- | B |
| 4446 | Ser-Cys]-NH2 | | | | 4149 | |
| 3BP- | DOTA-PPAc-Tyr-[Cys(3MeBn)-Glu-pro-Asp-Af3(Cpsu)-Leu- | 2139.874 | 2139.907 | 5.9 | 3BP- | A |
| 4448 | Thr-Trp-Ser-Cys]-NH2 | | | | 2776 | |
| 3BP- | DOTA-PPAc-Tyr-[Cys(3MeBn)-Glu-pro-Asp-Af3(Cpsu)-Npg- | 2153.890 | 2153.924 | 6.9 | 3BP | A |
| 4449 | Thr-Trp-Ser-Cys]-NH2 | | | | 2776 | |
| 3BP- | DOTA-PPAc-Ser-[Cys(3MeBn)-Glu-pro-Asp-Af3(Cpsu)-Leu- | 2063.843 | 2063.876 | 6.5 | 3BP- | A |
| 4450 | Thr-Trp-Ser-Cys]-NH2 | | | | 2776 | |
| 3BP- | DOTA-PPAc-Arg-[Cys(3MeBn)-Glu-pro-Asp-Af3(Cpsu)-Leu- | 2132.912 | 2132.950 | 6.1 | 3BP | A |
| 4451 | Thr-Trp-Ser-Cys]-NH2 | | | | 2776 | |
| 3BP- | DOTA-PPAc-Gln-[Cys(3MeBn)-Glu-pro-Asp-Af3(Cpsu)-Leu- | 2104.869 | 2104.900 | 6.5 | 3BP- | A |
| 4452 | Thr-Trp-Ser-Cys]-NH2 | | | | 2776 | |
| 3BP- | DOTA-Gln-[Cys(3MeBn)-Glu-pro-Asp-Af3(Cpsu)-Leu-Thr- | 1978.790 | 1978.820 | 6.4 | 3BP- | A |
| 4453 | Trp-Ser-Cys]-NH2 | | | | 2776 | |
| 3BP- | DOTA-Sni-Tyr-[Cys(3MeBn)-Glu-pro-Asp-Af3(Cpsu)-Leu- | 2124.863 | 2124.895 | 6.5 | 3BP- | B |
| 4454 | Thr-Trp-Ser-Cys]-NH2 | | | | 414 | |
| 3BP- | DOTA-Rni-Tyr-[Cys(3MeBn)-Glu-pro-Asp-Af3(Cpsu)-Leu- | 2124.863 | 2124.896 | 6.2 | 3BP- | A |
| 4455 | Thr-Trp-Ser-Cys]-NH2 | | | | 2776 | |
| 3BP- | DOTA-Pip-Tyr-[Cys(3MeBn)-Glu-pro-Asp-Af3(Cpsu)-Leu- | 2124.863 | 2124.900 | 6.3 | 3BP- | B |
| 4456 | Thr-Trp-Ser-Cys]-NH2 | | | | 4149 | |
| 3BP- | DOTA-PPAc-Tyr-[Cys(3MeBn)-Glu-pro-Asp-Aph(2Ta5Sa)- | 2179.815 | 2179.823 | 6.4 | 3BP- | A |
| 4457 | Leu-Thr-Trp-Ser-Cys]-NH2 | | | | 4149 | |
| 3BP- | DOTA-APAc-Val-Tyr-[Cys(3MeBn)-Glu-pro-Asp- | 2243.966 | 2244.029 | 6.3 | 3BP- | B |
| 4458 | Aph(HYDAc)-Leu-Thr-Trp-Ser-Cys]-NH2 | | | | 4149 | |
| 3BP- | DOTA-PPAc-Gln-[Cys(3MeBn)-Glu-pro-Asp-Aph(5SaPyr2)- | 2139.849 | 2139.874 | 6.7 | 3BP- | B |
| 4480 | Leu-Thr-Trp-Ser-Cys]-NH2 | | | | 4149 | |
| 3BP- | DOTA-PPAc-Gln-[Cys(3MeBn)-Glu-pro-Asp-Aph(6SaPyr3)- | 2139.849 | 2139.87 | 6.3 | 3BP- | C |
| 4481 | Leu-Thr-Trp-Ser-Cys]-NH2 | | | | 4149 | |
| 3BP- | DOTA-PPAc-Gln-[Cys(3MeBn)-Glu-pro-Asp- | 2142.860 | 2142.871 | 6.1 | 3BP- | C |
| 4482 | Aph(4SaPy2Ac)-Leu-Thr-Trp-Ser-Cys]-NH2 | | | | 4149 | |
| 3BP- | DOTA-{Asp-[Cys(3MeBn)-Glu-pro-Asp-Af3(Cpsu)-Leu-Thr- | 1946.764 | 1946.770 | 7.0 | 3BP- | A |
| 4488 | Trp-Dap}-Cys]-NH2 | | | | 4149 | |
| 3BP- | DOTA-{Asp-[Cys(3MeBn)-Glu-pro-Asp-Aph(SaPr)-Leu-Thr- | 1932.748 | 1932.759 | 6.5 | 3BP- | A |
| 4489 | Trp-Dap}-Cys]-NH2 | | | | 4149 | |
| 3BP- | DOTA-PPAc-{Asp-[Cys(3MeBn)-Glu-pro-Asp-Af3(Cpsu)-Leu- | 2072.843 | 2072.857 | 6.5 | 3BP- | B |
| 4497 | Thr-Trp-Dap}-Cys]-NH2 | | | | 2776 | |
| 3BP- | DOTA-PPAc-{Asp-[Cys(3MeBn)-Glu-pro-Asp-Aph(SaPr)- | 2058.827 | 2058.830 | 6.7 | 3BP- | B |
| 4498 | Leu-Thr-Trp-Dap}-Cys]-NH2 | | | | 4149 | |
| 3BP- | DOTA-PPAc-Gln-[Cys(3MeBn)-Glu-pro-Asp-Aph(SaPr)-Leu- | 2090.854 | 2090.868 | 6.1 | 3BP- | A |
| 4500 | Thr-Trp-Ser-Cys]-NH2 | | | | 2776 | |
| 3BP- | DOTA-Gln-[Cys(3MeBn)-Glu-pro-Asp-Aph(SaPr)-Leu-Thr- | 1964.774 | 1964.778 | 5.9 | 3BP- | A |
| 4501 | Trp-Ser-Cys]-NH2 | | | | 2776 | |
| 3BP- | DOTA-PPAc-{Glu-[Cys(3MeBn)-Glu-pro-Asp-Af3(Cpsu)-Leu- | 2086.859 | 2086.873 | 6.6 | 3BP- | A |
| 4502 | Thr-Trp-Dap}-Cys]-NH2 | | | | 2776 | |
| 3BP- | DOTA-{Glu-[Cys(3MeBn)-Glu-pro-Asp-Af3(Cpsu)-Leu-Thr- | 1960.779 | 1960.787 | 6.7 | 3BP- | A |
| 4503 | Trp-Dap}-Cys]-NH2 | | | | 2776 | |
| 3BP- | DOTA-PPAc-{Glu-[Cys(3MeBn)-Glu-pro-Asp-Aph(SaPr)-Leu- | 2072.843 | 2072.846 | 6.6 | 3BP | A |
| 4504 | Thr-Trp-Dap}-Cys]-NH2 | | | | 2776 | |
| 3BP- | DOTA-{Glu-[Cys(3MeBn)-Glu-pro-Asp-Aph(SaPr)-Leu-Thr- | 1946.764 | 1946.775 | 6.5 | 3BP- | A |
| 4505 | Trp-Dap}-Cys]-NH2 | | | | 2776 | |
| 3BP- | DOTA-PPAc-Gln-[Cys(3MeBn)-Glu-pro-Asp- | 2154.860 | 2154.905 | 6.8 | 3BP- | A |
| 4514 | Af3(2Py6SaNH)-Leu-Thr-Trp-Ser-Cys]-NH2 | | | | 2776 | |
| 3BP- | DOTA-PPAc-Gln-[Cys(3MeBn)-Glu-pro-Asp-Af3(PrHydr)- | 2084.897 | 2084.938 | 6.2 | 3BP- | B |
| 4515 | Leu-Thr-Trp-Ser-Cys]-NH2 | | | | 2776 | |
| 3BP- | GaDOTA-PPAc-Gln-[Cys(3MeBn)-Glu-pro-Asp-Af3(Cpsu)- | 2170.771 | 2170.777 | 6.1 | 3BP- | A |
| 4529 | Leu-Thr-Trp-Ser-Cys]-NH2 | | | | 2776 | |
| 3BP- | FAM-PPAc-Gln-[Cys(3MeBn)-Glu-pro-Asp-Af3(Cpsu)-Leu- | 2076.737 | 2076.718 | 7.3 | 3BP- | A |
| 4530 | Thr-Trp-Ser-Cys]-NH2 | | | | 2776 | |
| 3BP- | FAM-Ttds-PPAc-Gln-[Cys(3MeBn)-Glu-pro-Asp-Af3(Cpsu)- | 2378.921 | 2378.884 | 7.5 | 3BP- | A |
| 4531 | Leu-Thr-Trp-Ser-Cys]-NH2 | | | | 2776 | |
| 3BP- | FAM-Ttds-Ttds-PPAc-Gln-[Cys(3MeBn)-Glu-pro-Asp- | 2681.105 | 2681.060 | 7.5 | 3BP- | A |
| 4532 | Af3(Cpsu)-Leu-Thr-Trp-Ser-Cys]-NH2 | | | | 2776 | |
| 3BP- | FAM-Ttds-Ttds-Ttds-PPAc-Gln-[Cys(3MeBn)-Glu-pro-Asp- | 2983.289 | 2983.369 | 7.6 | 3BP- | A |
| 4548 | Af3(Cpsu)-Leu-Thr-Trp-Ser-Cys] | | | | 2776 | |
| 3BP- | InDOTA-PPAc-Gln-[Cys(3MeBn)-Glu-pro-Asp-Af3(Cpsu)- | 2214.750 | 2214.769 | 6.2 | 3BP- | A |
| 4554 | Leu-Thr-Trp-Ser-Cys]-NH2 | | | | 2776 | |
| 3BP- | LuDOTA-PPAc-Gln-[Cys(3MeBn)-Glu-pro-Asp-Af3(Cpsu)- | 2276.787 | 2276.806 | 6.4 | 3BP- | A |
| 4555 | Leu-Thr-Trp-Ser-Cys]-NH2 | | | | 2776 | |
| 3BP- | FITC-Ttds-PPAc-Gln-[Cys(3MeBn)-Glu-pro-Asp-Af3(Cpsu)- | 2409.909 | 2409.898 | 7.8 | 3BP- | A |
| 4578 | Leu-Thr-Trp-Ser-Cys]-NH2 | | | | 2776 | |
| 3BP- | AF488-Ttds-PPAc-Gln-[Cys(3MeBn)-Glu-pro-Asp-Af3(Cpsu)- | 2536.867 | 2536.863 | 6.4 | 3BP- | A |
| 4579 | Leu-Thr-Trp-Ser-Cys]-NH2 | | | | 2776 | |
| 3BP- | InDOTA-Gln-[Cys(3MeBn)-Glu-pro-Asp-Aph(SaPr)-Leu-Thr- | 2074.655 | 2074.656 | 6.5 | 3BP- | A |
| 4580 | Trp-Ser-Cys]-NH2 | | | | 2776 | |
| 3BP- | LuDOTA-Gln-[Cys(3MeBn)-Glu-pro-Asp-Aph(SaPr)-Leu-Thr- | 2136.692 | 2136.739 | 6.2 | 3BP- | A |
| 4581 | Trp-Ser-Cys]-NH2 | | | | 2776 | |
| 3BP- | GaDOTA-Gln-[Cys(3MeBn)-Glu-pro-Asp-Aph(SaPr)-Leu-Thr- | 2030.676 | 2030.704 | 5.9 | 3BP- | A |
| 4602 | Trp-Ser-Cys]-NH2 | | | | 2776 | |
| 3BP- | DOTA-PPAc-Gln-[Cys(3MeBn)-Glu-pro-Asp-Af3(Cshx)-Leu- | 2144.901 | 2145.031 | 5.9 | 3BP- | C |
| 4787 | Thr-Trp-Ser-Cys]-NH2 | | | | 2776 | |
| 3BP- | DOTA-PPAc-Gln-[Cys(3MeBn)-Glu-pro-Asp-Af3(Hsfu)-Leu- | 2128.833 | 2128.957 | 5.9 | 3BP- | A |
| 4788 | Thr-Trp-Ser-Cys]-NH2 | | | | 2776 | |
| 3BP- | DOTA-PPAc-Gln-[Cys(3MeBn)-Glu-pro-Asp-Af3(Hspy)-Leu- | 2127.849 | 2127.930 | 6.4 | 3BP- | B |
| 4789 | Thr-Trp-Ser-Cys]-NH2 | | | | 2776 | |
| 3BP- | DOTA-PPAc-Gln-[Cys(3MeBn)-Glu-pro-Asp-Af3(4SaPy2Ac)- | 2142.860 | 2142.980 | 5.6 | 3BP- | A |
| 4790 | Leu-Thr-Trp-Ser-Cys]-NH2 | | | | 2776 | |
| 3BP- | DOTA-PPAc-Gln-[Cys(3MeBn)-Glu-pro-Asp-Aph(Hsfu)-Leu- | 2128.833 | 2128.931 | 6.2 | 3BP- | B |
| 4813 | Thr-Trp-Ser-Cys]-NH2 | | | | 2776 | |
| 3BP- | DOTA-PPAc-Gln-[Cys(3MeBn)-Glu-pro-Asp-Aph(Hspy)-Leu- | 2127.849 | 2127.972 | 6.1 | 3BP- | C |
| 4814 | Thr-Trp-Ser-Cys]-NH2 | | | | 2776 | |
| 3BP- | DOTA-PPAc-Gln-[Cys(3MeBn)-Glu-pro-Asp-Af3(Aytr)-Leu- | 2123.883 | 2123.978 | 6.1 | 3BP- | C |
| 4847 | Thr-Trp-Ser-Cys]-NH2 | | | | 2776 | |
| 3BP- | DOTA-PPAc-Gln-[Cys(3MeBn)-Glu-pro-Asp-Af3(Hytr)-Leu- | 2123.883 | 2123.965 | 6.1 | 3BP- | C |
| 4882 | Thr-Trp-Ser-Cys]-NH2 | | | | 2776 | |
| 3BP- | DOTA-PPAc-Gln-[Cys(3MeBn)-Glu-pro-Asp-Aph(Aybu)-Leu- | 2084.897 | 2084.923 | 6.0 | 3BP- | C |
| 4884 | Thr-Trp-Ser-Cys]-NH2 | | | | 2776 | |
| 3BP- | DOTA-PPAc-Gln-[Cys(3MeBn)-Glu-pro-Asp-Af3(Aype)-Leu- | 2098.913 | 2099.041 | 5.9 | 3BP- | C |
| 4886 | Thr-Trp-Ser-Cys]-NH2 | | | | 2776 | |

### Example 16: Surface Plasmon Resonance Assay

Surface plasmon resonance (SPR) studies were performed using a Biacore™ T200 SPR system (GE Healthcare Life Sciences). Briefly, polarized light is directed towards a gold-labeled sensor surface, and minimum intensity reflected light is detected. The angle of reflected light changes as molecules bind and dissociate.

Fc-fusion protein of human carbonic anhydrase IX (hCAIX-Fc, SinoBliological, Cat #10107-H02H) was captured on a Fc-capture chip (Biacore™ CM5 sensor chip coated with ˜300 RU of an Fc-binding peptide). Recombinant carbonic anydrase was diluted in Running Buffer (PBST, 0.1% DMSO) to a final concentration 100 or 200 nM and than flushed over the Fc-capture chip to immobilized ˜1000 RUs.

Stock solutions of test compounds were prepared by dissolving each compound in DMSO. DMSO stock solution were diluted 1:1000 in Running Buffer without DMSO. Further sequencial dilutions were made with Running Buffer containing 0.1% DMSO. SPR binding analyses were performed in Single Cycle Kinetic (SCK) mode at 25° C. Flow cell coated with the Fc-binding peptide only served as reference flowcell. After each SCK run, carbonic andydrase IX was removed with 10 mM glycine buffer, pH 1.5.

In between every three SCK measurements, a blank run with Running Buffer instead of test compound was included to correct for baseline drifts (double blanking methods).

Table 11 describes the protocol steps for Fc-fusion target capturing and assessment of the binding kinetics.

TABLE 11
SPR protocol steps with hCAIX-Fc.

| | | Contact | | Flow | |
|---|---|---|---|---|---|
| Step | Injected solution | time | | rate | |
| Startup cycle (3x): | PBST, 0.1% DMSO Buffer | 60 | s | 30 | μL/min |
| Washing & surface regeneration | 10 mM glycine, pH 1.5 | 5 | s | | |
| Caputure target protein | 200 nM hCAIX-Fc | 300 | s | 5 | μL/min |
| 1. Binding kinetics of test compound | Dilution no. 5 (e.g. 0.2 nM) | 120 | s | 30 | μL/min |
| 2. Binding kinetics of test compound | Dilution no. 4 (e.g. 0.8 nM) | 120 | s | 30 | μL/min |
| 3. Binding kinetics of test compound | Dilution no. 3 (e.g. 3.1 nM) | 120 | s | 30 | μL/min |
| 4. Binding kinetics of test compound | Dilution no. 2 (e.g. 12.5 nM) | 120 | s | 30 | μL/min |
| 5. Binding kinetics of test compound | Dilution no. 1 (e.g. 50 nM) | 120 | s | 30 | μL/min |
| Dissociation cycle | PBST, 0.1% DMSO Buffer | 1200 | s | 30 | μL/min |
| Regeneration (2x) | 10 mM glycine, pH 1.5 | 20 | s | 30 | μL/min |

For each test compound, SPR raw data in the form of resonance units (RU) were plotted as sensorgrams using the Biacore™ T200 control software. The signal from the blank sensorgram was subtracted from that of the test compound sensorgram (blank corrected). The blank corrected sensorgram was corrected for baseline drift by subtracting the sensorgram of a SCK run without the test compound (running buffer only). The association rate (kₒₙ), dissociation rate (k_{off}), dissociation constant (K_{D}), and t_{1/2} were calculated from Blank-normalized SPR data using the 1:1 Langmuir binding model from the Biacore™ T200 evaluation software. Raw data and fit results were imported as text files in IDBS. The pK_{D} value (negative decadic logarithm of dissociation constant) was calculated in the IDBS excel template.

The results of this assay for exemplary of compounds of the present invention are presented in Table 12. pK_{D} category A stands for pK_{D} values >8.0, category B for pK_{D} values between 7.1 and 8.0, and category C for pK_{D} values between 6.1 and 7.0. Half-life (t_{1/2}) category A stands for t_{1/2}>15.0 minutes, category B for t_{1/2} between 5.1 and 15.0 minutes, category C for t_{1/2} between 2.1 and 5.0 minutes, and category D for t_{1/2}≤2.0 minutes.

TABLE 12
Compound ID, pK_{D} and half-life category of CAIX SPR assay.

| | | pK_{D} | t_{1/2} |
|---|---|---|---|
| | | cate- | cate- |
| | | gory | gory |
| | | (activ- | (activ- |
| ID | Sequence | ity) | ity) |
| 3BP-2959 | DOTA-Ttds-Val-Tyr-[Cys(3MeBn)-Glu-Gly-Asp-Trp-Leu-Thr-Trp-Gln- | A | D |
| | Cys]-NH2 | | |
| 3BP-3058 | Ac-Val-Tyr-[Cys(3MeBn)-Glu-Gly-Asp-Trp-Leu-Thr-Trp-Ala-Cys]-NH2 | A | C |
| 3BP-3271 | DOTA-Ttds-Val-Tyr-[Cys(3MeBn)-Glu-Nmg-Asp-Trp-Leu-Thr-Trp-Gln- | A | C |
| | Cys]-NH2 | | |
| 3BP-3272 | DOTA-Ttds-Val-Tyr-[Cys(3MeBn)-Glu-Gly-Asp-Trp-Leu-Thr-Trp-Ala- | A | C |
| | Cys]-NH2 | | |
| 3BP-3279 | DOTA-Ttds-Val-Tyr-[Cys(3MeBn)-Glu-ala-Asp-Trp-Leu-Thr-Trp-Ala- | A | B |
| | Cys]-NH2 | | |
| 3BP-3280 | Ac-Val-Tyr-[Cys(3MeBn)-Glu-ala-Asp-Trp-Leu-Thr-Trp-Ala-Cys]-NH2 | A | B |
| 3BP-3281 | Ac-Val-Tyr-[Cys(3MeBn)-Glu-nma-Asp-Trp-Leu-Thr-Trp-Ala-Cys]-NH2 | A | B |
| 3BP-3282 | Ac-Val-Tyr-[Cys(3MeBn)-Glu-pro-Asp-Trp-Leu-Thr-Trp-Ala-Cys]-NH2 | A | A |
| 3BP-3283 | Ac-Val-Tyr-[Cys(3MeBn)-Glu-pip-Asp-Trp-Leu-Thr-Trp-Ala-Cys]-NH2 | A | B |
| 3BP-3289 | Ac-Val-Tyr-[Cys(3MeBn)-Glu-Nmg-Asp-5Clw-Leu-Thr-Trp-Gln-Cys]-NH2 | A | B |
| 3BP-3311 | Ac-Val-Tyr-[Cys(3MeBn)-Glu-Nmg-Asp-Bta-Leu-Thr-Trp-Gln-Cys]-NH2 | A | C |
| 3BP-3313 | Ac-Val-Tyr-[Cys(3MeBn)-Glu-Nmg-Asp-Trp-Leu-Thr-Trp-Ala-Cys]-NH2 | A | C |
| 3BP-3328 | InDOTA-Ttds-Val-Tyr-[Cys(3MeBn)-Glu-Gly-Asp-Trp-Leu-Thr-Trp-Gln- | A | B |
| | Cys]-NH2 | | |
| 3BP-3432 | DOTA-APAc-Val-Tyr-[Cys(3MeBn)-Glu-pro-Asp-Trp-Leu-Thr-Trp-Ala- | A | A |
| | Cys]-NH2 | | |
| 3BP-3478 | Ac-Val-Tyr-[Cys(3MeBn)-Glu-pro-Asp-Trp-Leu-Thr-Trp-Glu(NH-Apr- | A | A |
| | DOTA′)-Cys]-NH2 | | |
| 3BP-3562 | Ac-Val-Tyr-[Cys(3MeBn)-Glu-Nlys(DOTA)-Asp-Trp-Leu-Thr-Trp-Ala- | A | A |
| | Cys]-NH2 | | |
| 3BP-3565 | Ac-Val-Tyr-[Cys(3MeBn)-Glu-pro-Asp-Trp-Leu-Thr-Trp-Ser-Cys]-NH2 | A | A |
| 3BP-3583 | DOTA-APAc-Val-Tyr-[Cys(3MeBn)-Glu-pro-Asp-Trp-Leu-Thr-Trp-Ser- | A | A |
| | Cys]-NH2 | | |
| 3BP-3584 | DOTA-APAc-Val-Tyr-[Cys(3MeBn)-Glu-pro-Asp-Trp-Egz-Thr-Trp-Ser- | A | B |
| | Cys]-NH2 | | |
| 3BP-3587 | DOTA-APAc-Val-Tyr-[Cys(3MeBn)-Glu-pro-Asp-Trp-Eca-Thr-Trp-Ser- | A | C |
| | Cys]-NH2 | | |
| 3BP-3599 | DOTA-APAc-Val-Tyr-[Cys(3MeBn)-Glu-pro-Asp-Trp-Leu-Thr-Trp-Arg- | A | B |
| | Cys]-NH2 | | |
| 3BP-3732 | Ac-Ser-Tyr-[Cys(3MeBn)-Glu-pro-Asp-Trp-Leu-Thr-Trp-Glu(NH-Apr- | A | B |
| | O2Oc-DOTA′)-Cys]-NH2 | | |
| 3BP-3734 | Ac-Val-Arg-[Cys(3MeBn)-Glu-pro-Asp-Trp-Leu-Thr-Trp-Glu(NH-Apr- | A | B |
| | O2Oc-DOTA′)-Cys]-NH2 | | |
| 3BP-3740 | Iva-Tyr-[Cys(3MeBn)-Glu-pro-Asp-Trp-Leu-Thr-Trp-Glu(NH-Apr- | A | B |
| | O2Oc-DOTA′)-Cys]-NH2 | | |
| 3BP-3840 | Ac-Lys(DOTA)-Val-Tyr-[Cys(3MeBn)-Glu-pro-Asp-Trp-Leu-Thr-Trp- | A | B |
| | Ser-Cys]-NH2 | | |
| 3BP-3842 | 3OHPr-Tyr-[Cys(3MeBn)-Glu-pro-Asp-Trp-Leu-Thr-Trp-Glu(NH-Apr- | A | C |
| | DOTA′)-Cys]-NH2 | | |
| 3BP-3858 | DOTA-APAc-[Cys(3MeBn)-Glu-pro-Asp-Trp-Leu-Thr-Trp-Ser-Cys]-NH2 | A | D |
| 3BP-3859 | DOTA-Tyr-[Cys(3MeBn)-Glu-pro-Asp-Trp-Leu-Thr-Trp-Ser-Cys]-NH2 | A | B |
| 3BP-3867 | DOTA-APAc-Tyr-[Cys(3MeBn)-Glu-pro-Asp-Trp-Leu-Thr-Trp-Ser-Cys]- | A | C |
| | NH2 | | |
| 3BP-3868 | DOTA-4Amc-Tyr-[Cys(3MeBn)-Glu-pro-Asp-Trp-Leu-Thr-Trp-Ser-Cys]- | A | B |
| | NH2 | | |
| 3BP-3869 | DOTA-O2Oc-Tyr-[Cys(3MeBn)-Glu-pro-Asp-Trp-Leu-Thr-Trp-Ser-Cys]- | B | C |
| | NH2 | | |
| 3BP-3870 | DOTA-Ttds-Nle-Tyr-[Cys(3MeBn)-Glu-pro-Asp-Trp-Leu-Thr-Trp-Ser- | A | B |
| | Cys]-NH2 | | |
| 3BP-3873 | Ac-Ser-Tyr-[Cys(3MeBn)-Glu-pro-Asp-Trp-Leu-Thr-Trp-Glu(NH-Apr- | A | B |
| | DOTA′)-Cys]-NH2 | | |
| 3BP-3874 | Ac-Val-Phe-[Cys(3MeBn)-Glu-pro-Asp-Trp-Leu-Thr-Trp-Glu(NH-Apr- | A | B |
| | DOTA′)-Cys]-NH2 | | |
| 3BP-3905 | InDOTA-APAc-Val-Tyr-[Cys(3MeBn)-Glu-pro-Asp-Trp-Leu-Thr-Trp-Ser- | A | A |
| | Cys]-NH2 | | |
| 3BP-3906 | InDOTA-Tyr-[Cys(3MeBn)-Glu-pro-Asp-Trp-Leu-Thr-Trp-Ser-Cys]-NH2 | A | C |
| 3BP-3934 | DOTA-Tyr-[Cys(3MeBn)-Glu-pro-{Lys-Trp-Leu-Glu}-Trp-Ser-Cys]-NH2 | A | C |
| 3BP-4053 | InDOTA-Tyr-[Cys(3MeBn)-Glu-pro-{Lys-Trp-Leu-Glu}-Trp-Ser-Cys]- | A | B |
| | NH2 | | |
| 3BP-4095 | DOTA-APAc-Val-Dmo-[Cys(3MeBn)-Glu-pro-Asp-Trp-Leu-Thr-Trp-Ser- | A | B |
| | Cys]-NH2 | | |
| 3BP-4096 | DOTA-APAc-Val-Tyr-[Cys(3MeBn)-Dmo-pro-Asp-Trp-Leu-Thr-Trp-Ser- | A | B |
| | Cys]-NH2 | | |
| 3BP-4097 | DOTA-APAc-Val-Tyr-[Cys(3MeBn)-Glu-dmo-Asp-Trp-Leu-Thr-Trp-Ser- | A | B |
| | Cys]-NH2 | | |
| 3BP-4101 | DOTA-APAc-Val-Tyr-[Cys(3MeBn)-Hse-pro-Asp-Trp-Leu-Thr-Trp-Ser- | A | B |
| | Cys]-NH2 | | |
| 3BP-4102 | DOTA-APAc-Val-Tyr-[Cys(3MeBn)-Glu-pro-Hse-Trp-Leu-Thr-Trp-Ser- | A | A |
| | Cys]-NH2 | | |
| 3BP-4103 | DOTA-APAc-Val-Tyr-[Cys(3MeBn)-Glu-pro-Asn-Trp-Leu-Thr-Trp-Ser- | A | A |
| | Cys]-NH2 | | |
| 3BP-4104 | DOTA-APAc-Val-Tyr-[Cys(3MeBn)-Gln-pro-Asp-Trp-Leu-Thr-Trp-Ser- | A | B |
| | Cys]-NH2 | | |
| 3BP-4105 | DOTA-APAc-Val-Ser-[Cys(3MeBn)-Glu-pro-Asp-Trp-Leu-Thr-Trp-Ser- | A | B |
| | Cys]-NH2 | | |
| 3BP-4107 | DOTA-APAc-Val-Tyr-[Cys(3MeBn)-Glu-pro-Nmd-Trp-Leu-Thr-Trp-Ser- | A | A |
| | Cys]-NH2 | | |
| 3BP-4112 | DOTA-APAc-Val-Glu(AGLU′)-[Cys(3MeBn)-Glu-pro-Asp-Trp-Leu-Thr- | A | B |
| | Trp-Ser-Cys]-NH2 | | |
| 3BP-4119 | DOTA-APAc-Val-Tyr-[Cys(3MeBn)-Glu-glu(AGLU′)-Asp-Trp-Leu-Thr- | A | B |
| | Trp-Ser-Cys]-NH2 | | |
| 3BP-4121 | DOTA-APAc-Val-Tyr-[Cys(3MeBn)-Glu-pro-Asp-Trp-Leu-Glu(AGLU′)- | A | A |
| | Trp-Ser-Cys]-NH2 | | |
| 3BP-4122 | DOTA-APAc-Val-Tyr-[Cys(3MeBn)-Glu-pro-Asp-Trp-Leu-Thr-Trp- | A | A |
| | Glu(AGLU′)-Cys]-NH2 | | |
| 3BP-4123 | DOTA-APAc-Val-Tyr-[Cys(3MeBn)-Glu-pro-Eem-Trp-Leu-Thr-Trp-Ser- | A | A |
| | Cys]-NH2 | | |
| 3BP-4128 | DOTA-APAc-Val-Tyr-[Cys(3MeBn)-Glu-pro-Cys(Bzl)-Trp-Leu-Thr-Trp- | A | A |
| | Ser-Cys]-NH2 | | |
| 3BP-4133 | DOTA-APAc-Val-Tyr-[Cys(3MeBn)-Glu-pro-Asp-Eaa-Leu-Thr-Trp-Ser- | A | B |
| | Cys]-NH2 | | |
| 3BP-4134 | DOTA-APAc-Val-Tyr-[Cys(3MeBn)-Glu-pro-Asp-Pif-Leu-Thr-Trp-Ser- | A | C |
| | Cys]-NH2 | | |
| 3BP-4135 | DOTA-APAc-Val-Tyr-[Cys(3MeBn)-Glu-pro-Asp-Mtf-Leu-Thr-Trp-Ser- | A | B |
| | Cys]-NH2 | | |
| 3BP-4139 | DOTA-APAc-Val-Tyr-[Cys(3MeBn)-Glu-pro-Asp-Ptf-Leu-Thr-Trp-Ser- | A | C |
| | Cys]-NH2 | | |
| 3BP-4142 | DOTA-APAc-Val-Tyr-[Cys(3MeBn)-Glu-pro-Asp-Trp-Npg-Thr-Trp-Ser- | A | A |
| | Cys]-NH2 | | |
| 3BP-4143 | DOTA-APAc-Val-Tyr-[Cys(3MeBn)-Glu-pro-Asp-Trp-Cha-Thr-Trp-Ser- | A | C |
| | Cys]-NH2 | | |
| 3BP-4144 | DOTA-APAc-Val-Tyr-[Cys(3MeBn)-Glu-pro-Asp-1MW-Leu-Thr-Trp-Ser- | A | B |
| | Cys]-NH2 | | |
| 3BP-4158 | DOTA-APAc-Val-Tyr-[Cys(3MeBn)-Glu-pro-Asp-7MW-Leu-Thr-Trp-Ser- | A | B |
| | Cys]-NH2 | | |
| 3BP-4174 | DOTA-APAc-Val-{Asp-[Cys(3MeBn)-Glu-pro-Asp-Trp-Leu-Thr-Trp-Dap}- | A | A |
| | Cys]-NH2 | | |
| 3BP-4175 | DOTA-APAc-Val-{Glu-[Cys(3MeBn)-Glu-pro-Asp-Trp-Leu-Thr-Trp-Dap}- | A | A |
| | Cys]-NH2 | | |
| 3BP-4176 | DOTA-APAc-Val-{Dap-[Cys(3MeBn)-Glu-pro-Asp-Trp-Leu-Thr-Trp-Asp}- | A | C |
| | Cys]-NH2 | | |
| 3BP-4236 | DOTA-APAc-Val-Tyr-[Cys(3MeBn)-Glu-pro-Asp-Af3(Iva)-Leu-Thr-Trp- | A | B |
| | Ser-Cys]-NH2 | | |
| 3BP-4237 | DOTA-APAc-Val-Tyr-[Cys(3MeBn)-Glu-pro-Asp-Af3(HO-Succinyl)-Leu- | A | A |
| | Thr-Trp-Ser-Cys]-NH2 | | |
| 3BP-4242 | DOTA-APAc-Val-Tyr-[Cys(3MeBn)-Glu-pro-Asp-Af3(MSAc)-Leu-Thr-Trp- | A | B |
| | Ser-Cys]-NH2 | | |
| 3BP-4253 | DOTA-{Asp-[Cys(3MeBn)-Glu-pro-Asp-Trp-Leu-Thr-Trp-Dap}-Cys]-NH2 | A | B |
| 3BP-4254 | DOTA-APAc-{Asp-[Cys(3MeBn)-Glu-pro-Asp-Trp-Leu-Thr-Trp-Dap}-Cys]- | A | C |
| | NH2 | | |
| 3BP-4255 | DOTA-PPAc-{Asp-[Cys(3MeBn)-Glu-pro-Asp-Trp-Leu-Thr-Trp-Dap}-Cys]- | A | D |
| | NH2 | | |
| 3BP-4256 | DOTA-Inp-{Asp-[Cys(3MeBn)-Glu-pro-Asp-Trp-Leu-Thr-Trp-Dap}-Cys]- | A | C |
| | NH2 | | |
| 3BP-4257 | DOTA-Ser-{Asp-[Cys(3MeBn)-Glu-pro-Asp-Trp-Leu-Thr-Trp-Dap}-Cys]- | A | C |
| | NH2 | | |
| 3BP-4258 | DOTA-4Amc-{Asp-[Cys(3MeBn)-Glu-pro-Asp-Trp-Leu-Thr-Trp-Dap}-Cys]- | A | C |
| | NH2 | | |
| 3BP-4259 | DOTA-Cmp-{Asp-[Cys(3MeBn)-Glu-pro-Asp-Trp-Leu-Thr-Trp-Dap}-Cys]- | A | D |
| | NH2 | | |
| 3BP-4260 | DOTA-Mamb-{Asp-[Cys(3MeBn)-Glu-pro-Asp-Trp-Leu-Thr-Trp-Dap}- | A | D |
| | Cys]-NH2 | | |
| 3BP-4261 | DOTA-Pamb-{Asp-[Cys(3MeBn)-Glu-pro-Asp-Trp-Leu-Thr-Trp-Dap}- | A | C |
| | Cys]-NH2 | | |
| 3BP-4263 | DOTA-Gab-{Asp-[Cys(3MeBn)-Glu-pro-Asp-Trp-Leu-Thr-Trp-Dap}-Cys]- | A | C |
| | NH2 | | |
| 3BP-4264 | DOTA-Val-{Asp-[Cys(3MeBn)-Glu-pro-Asp-Trp-Leu-Thr-Trp-Dap}-Cys]- | A | A |
| | NH2 | | |
| 3BP-4288 | DOTA-APAc-Val-Tyr-[Cys(3MeBn)-Glu-pro-Asp-Af3(DkpAc)-Leu-Thr- | B | D |
| | Trp-Ser-Cys]-NH2 | | |
| 3BP-4305 | DOTA-APAc-Val-Tyr-[Cys(3MeBn)-Glu-pro-Asp-Af3(CMPy)-Leu-Thr-Trp- | B | D |
| | Ser-Cys]-NH2 | | |
| 3BP-4322 | DOTA-APAc-Val-Tyr-[Cys(3MeBn)-Glu-pro-Asp-Af3(HySuc)-Leu-Thr- | A | B |
| | Trp-Ser-Cys]-NH2 | | |
| 3BP-4324 | DOTA-APAc-Val-Tyr-[Cys(3MeBn)-Glu-pro-Asp-Af3(TzPr)-Leu-Thr-Trp- | A | A |
| | Ser-Cys]-NH2 | | |
| 3BP-4334 | DOTA-APAc-Val-Tyr-[Cys(3MeBn)-Glu-pro-Glu(N4BzICI)-Trp-Leu-Thr- | A | A |
| | Trp-Ser-Cys]-NH2 | | |
| 3BP-4337 | InDOTA-APAc-Val-{Asp-[Cys(3MeBn)-Glu-pro-Asp-Trp-Leu-Thr-Trp- | A | A |
| | Dap}-Cys]-NH2 | | |
| 3BP-4338 | InDOTA-{Asp-[Cys(3MeBn)-Glu-pro-Asp-Trp-Leu-Thr-Trp-Dap}-Cys]- | A | D |
| | NH2 | | |
| 3BP-4341 | DOTA-APAc-Val-Tyr-[Cys(3MeBn)-Glu-pro-Asp-Af3(DlmAc)-Leu-Thr- | A | C |
| | Trp-Ser-Cys]-NH2 | | |
| 3BP-4356 | DOTA-APAc-Val-Tyr-[Cys(3MeBn)-Glu-pro-Asp-Aph(HO-Succinyl)-Leu- | B | D |
| | Thr-Trp-Ser-Cys]-NH2 | | |
| 3BP-4361 | DOTA-APAc-Val-Tyr-[Cys(3MeBn)-Glu-pro-Asp-Af3(Ac-Glu)-Leu-Thr- | A | A |
| | Trp-Ser-Cys]-NH2 | | |
| 3BP-4362 | DOTA-APAc-Val-Tyr-[Cys(3MeBn)-Glu-pro-Asp-af3(ac-glu)-Leu-Thr- | A | B |
| | Trp-Ser-Cys]-NH2 | | |
| 3BP-4363 | DOTA-APAc-Val-Tyr-[Cys(3MeBn)-Glu-pro-Asp-Af3(Ac-Asp)-Leu-Thr- | A | B |
| | Trp-Ser-Cys]-NH2 | | |
| 3BP-4364 | DOTA-APAc-Val-Tyr-[Cys(3MeBn)-Glu-pro-Asp-af3(ac-asp)-Leu-Thr- | A | B |
| | Trp-Ser-Cys]-NH2 | | |
| 3BP-4367 | DOTA-APAc-Val-Tyr-[Cys(3MeBn)-Glu-pro-Asp-Af3(HO-Glutar)-Leu- | A | A |
| | Thr-Trp-Ser-Cys]-NH2 | | |
| 3BP-4368 | DOTA-APAc-Val-Tyr-[Cys(3MeBn)-Glu-pro-Asp-Af3(HO-Dga)-Leu-Thr- | A | C |
| | Trp-Ser-Cys]-NH2 | | |
| 3BP-4369 | DOTA-APAc-Val-Tyr-[Cys(3MeBn)-Glu-pro-Asp-Af3(Cpsu)-Leu-Thr-Trp- | A | A |
| | Ser-Cys]-NH2 | | |
| 3BP-4371 | DOTA-APAc-Val-Tyr-[Cys(3MeBn)-Glu-pro-Asp-Af3(HYDAc)-Leu-Thr- | A | C |
| | Trp-Ser-Cys]-NH2 | | |
| 3BP-4377 | DOTA-APAc-Val-{Asp-[Cys(3MeBn)-Glu-pro-Asp-Af3(HO-Succinyl)-Leu- | A | A |
| | Thr-Trp-Dap}-Cys]-NH2 | | |
| 3BP-4387 | DOTA-APAc-Val-Tyr-[Cys(3MeBn)-Glu-pro-Glu(N4Inda)-Trp-Leu-Thr- | A | A |
| | Trp-Ser-Cys]-NH2 | | |
| 3BP-4398 | DOTA-APAc-Val-Tyr-[Cys(3MeBn)-Glu-pro-Asp-Aph(4SaBz)-Leu-Thr- | A | C |
| | Trp-Ser-Cys]-NH2 | | |
| 3BP-4400 | DOTA-APAc-Val-Tyr-[Cys(3MeBn)-Glu-pro-Asp-Aph(SaPr)-Leu-Thr- | A | A |
| | Trp-Ser-Cys]-NH2 | | |
| 3BP-4401 | DOTA-APAc-Val-Tyr-[Cys(3MeBn)-Glu-pro-Asp-Aph(3Ta5Sa)-Leu-Thr- | A | A |
| | Trp-Ser-Cys]-NH2 | | |
| 3BP-4402 | DOTA-APAc-Val-Tyr-[Cys(3MeBn)-Glu-pro-Asp-Aph(2Ta5Sa)-Leu-Thr- | A | A |
| | Trp-Ser-Cys]-NH2 | | |
| 3BP-4405 | DOTA-APAc-Val-Tyr-[Cys(3MeBn)-Glu-pro-Asp-Af3(4SaBz)-Leu-Thr- | A | A |
| | Trp-Ser-Cys]-NH2 | | |
| 3BP-4406 | DOTA-APAc-Val-Tyr-[Cys(3MeBn)-Glu-pro-Asp-Af3(3MSaBz)-Leu-Thr- | A | A |
| | Trp-Ser-Cys]-NH2 | | |
| 3BP-4407 | DOTA-APAc-Val-Tyr-[Cys(3MeBn)-Glu-pro-Asp-Af3(SaPr)-Leu-Thr- | A | A |
| | Trp-Ser-Cys]-NH2 | | |
| 3BP-4408 | DOTA-APAc-Val-Tyr-[Cys(3MeBn)-Glu-pro-Asp-Aph(3SaBz)-Leu-Thr- | A | A |
| | Trp-Ser-Cys]-NH2 | | |
| 3BP-4409 | DOTA-APAc-Val-Tyr-[Cys(3MeBn)-Glu-pro-Asp-Af3(3SaBz)-Leu-Thr- | A | A |
| | Trp-Ser-Cys]-NH2 | | |
| 3BP-4412 | DOTA-APAc-Val-Tyr-[Cys(3MeBn)-Glu-pro-Asp-Af3(3Ta5Sa)-Leu-Thr- | A | A |
| | Trp-Ser-Cys]-NH2 | | |
| 3BP-4413 | DOTA-APAc-Val-Tyr-[Cys(3MeBn)-Glu-pro-Asp-Af3(2Ta5Sa)-Leu-Thr- | A | A |
| | Trp-Ser-Cys]-NH2 | | |
| 3BP-4439 | DOTA-APAc-Val-{Asp-[Cys(3MeBn)-Glu-pro-Asp-Eaa-Leu-Thr-Trp-Dap}- | A | B |
| | Cys]-NH2 | | |
| 3BP-4440 | DOTA-APAc-Val-{Asp-[Cys(3MeBn)-Glu-pro-Asp-Mtf-Leu-Thr-Trp-Dap}- | A | B |
| | Cys]-NH2 | | |
| 3BP-4441 | DOTA-APAc-Val-Tyr-[Cys(3MeBn)-Glu-pro-Glu(NH3PhSa)-Trp-Leu-Thr- | A | A |
| | Trp-Ser-Cys]-NH2 | | |
| 3BP-4442 | DOTA-APAc-Val-Tyr-[Cys(3MeBn)-Glu-pro-Glu(NH4PhSa)-Trp-Leu-Thr- | A | A |
| | Trp-Ser-Cys]-NH2 | | |
| 3BP-4445 | DOTA-APAc-Val-{Asp-[Cys(3MeBn)-Glu-pro-Asp-Af3(Cpsu)-Leu-Thr- | A | A |
| | Trp-Dap}-Cys]-NH2 | | |
| 3BP-4446 | DOTA-PPAc-Tyr-[Cys(3MeBn)-Glu-pro-Asp-Trp-Leu-Thr-Trp-Ser-Cys]- | A | C |
| | NH2 | | |
| 3BP-4448 | DOTA-PPAc-Tyr-[Cys(3MeBn)-Glu-pro-Asp-Af3(Cpsu)-Leu-Thr-Trp-Ser- | A | A |
| | Cys]-NH2 | | |
| 3BP-4452 | DOTA-PPAc-Gln-[Cys(3MeBn)-Glu-pro-Asp-Af3(Cpsu)-Leu-Thr-Trp-Ser- | A | A |
| | Cys]-NH2 | | |
| 3BP-4453 | DOTA-Gln-[Cys(3MeBn)-Glu-pro-Asp-Af3(Cpsu)-Leu-Thr-Trp-Ser-Cys]- | A | A |
| | NH2 | | |
| 3BP-4454 | DOTA-Sni-Tyr-[Cys(3MeBn)-Glu-pro-Asp-Af3(Cpsu)-Leu-Thr-Trp-Ser- | A | A |
| | Cys]-NH2 | | |
| 3BP-4455 | DOTA-Rni-Tyr-[Cys(3MeBn)-Glu-pro-Asp-Af3(Cpsu)-Leu-Thr-Trp-Ser- | A | A |
| | Cys]-NH2 | | |
| 3BP-4456 | DOTA-Pip-Tyr-[Cys(3MeBn)-Glu-pro-Asp-Af3(Cpsu)-Leu-Thr-Trp-Ser- | A | A |
| | Cys]-NH2 | | |
| 3BP-4457 | DOTA-PPAc-Tyr-[Cys(3MeBn)-Glu-pro-Asp-Aph(2Ta5Sa)-Leu-Thr-Trp- | A | B |
| | Ser-Cys]-NH2 | | |
| 3BP-4480 | DOTA-PPAc-Gln-[Cys(3MeBn)-Glu-pro-Asp-Aph(5SaPyr2)-Leu-Thr-Trp- | A | C |
| | Ser-Cys]-NH2 | | |
| 3BP-4488 | DOTA-{Asp-[Cys(3MeBn)-Glu-pro-Asp-Af3(Cpsu)-Leu-Thr-Trp-Dap}- | A | A |
| | Cys]-NH2 | | |
| 3BP-4489 | DOTA-{Asp-[Cys(3MeBn)-Glu-pro-Asp-Aph(SaPr)-Leu-Thr-Trp-Dap}- | A | A |
| | Cys]-NH2 | | |
| 3BP-4497 | DOTA-PPAc-{Asp-[Cys(3MeBn)-Glu-pro-Asp-Af3(Cpsu)-Leu-Thr-Trp- | A | A |
| | Dap}-Cys]-NH2 | | |
| 3BP-4498 | DOTA-PPAc-{Asp-[Cys(3MeBn)-Glu-pro-Asp-Aph(SaPr)-Leu-Thr-Trp- | A | A |
| | Dap}-Cys]-NH2 | | |
| 3BP-4500 | DOTA-PPAc-Gln-[Cys(3MeBn)-Glu-pro-Asp-Aph(SaPr)-Leu-Thr-Trp-Ser- | A | A |
| | Cys]-NH2 | | |
| 3BP-4501 | DOTA-Gln-[Cys(3MeBn)-Glu-pro-Asp-Aph(SaPr)-Leu-Thr-Trp-Ser-Cys]- | A | A |
| | NH2 | | |
| 3BP-4502 | DOTA-PPAc-{Glu-[Cys(3MeBn)-Glu-pro-Asp-Af3(Cpsu)-Leu-Thr-Trp- | A | A |
| | Dap}-Cys]-NH2 | | |
| 3BP-4503 | DOTA-{Glu-[Cys(3MeBn)-Glu-pro-Asp-Af3(Cpsu)-Leu-Thr-Trp-Dap}- | A | A |
| | Cys]-NH2 | | |
| 3BP-4504 | DOTA-PPAc-{Glu-[Cys(3MeBn)-Glu-pro-Asp-Aph(SaPr)-Leu-Thr-Trp- | A | A |
| | Dap}-Cys]-NH2 | | |
| 3BP-4505 | DOTA-{Glu-[Cys(3MeBn)-Glu-pro-Asp-Aph(SaPr)-Leu-Thr-Trp-Dap}- | A | A |
| | Cys]-NH2 | | |
| 3BP-4514 | DOTA-PPAc-Gln-[Cys(3MeBn)-Glu-pro-Asp-Af3(2Py6SaNH)-Leu-Thr- | A | B |
| | Trp-Ser-Cys]-NH2 | | |
| 3BP-4515 | DOTA-PPAc-Gln-[Cys(3MeBn)-Glu-pro-Asp-Af3(PrHydr)-Leu-Thr-Trp- | A | C |
| | Ser-Cys]-NH2 | | |
| 3BP-4529 | GaDOTA-PPAc-Gln-[Cys(3MeBn)-Glu-pro-Asp-Af3(Cpsu)-Leu-Thr-Trp- | A | A |
| | Ser-Cys]-NH2 | | |
| 3BP-4555 | LuDOTA-PPAc-Gln-[Cys(3MeBn)-Glu-pro-Asp-Af3(Cpsu)-Leu-Thr-Trp- | A | A |
| | Ser-Cys]-NH2 | | |
| 3BP-4581 | LuDOTA-Gln-[Cys(3MeBn)-Glu-pro-Asp-Aph(SaPr)-Leu-Thr-Trp-Ser- | A | A |
| | Cys]-NH2 | | |
| 3BP-4602 | GaDOTA-Gln-[Cys(3MeBn)-Glu-pro-Asp-Aph(SaPr)-Leu-Thr-Trp-Ser- | A | A |
| | Cys]-NH2 | | |
| 3BP-4788 | DOTA-PPAc-Gln-[Cys(3MeBn)-Glu-pro-Asp-Af3(Hsfu)-Leu-Thr-Trp-Ser- | A | A |
| | Cys]-NH2 | | |
| 3BP-4789 | DOTA-PPAc-Gln-[Cys(3MeBn)-Glu-pro-Asp-Af3(Hspy)-Leu-Thr-Trp-Ser- | B | C |
| | Cys]-NH2 | | |
| 3BP-4790 | DOTA-PPAc-Gln-[Cys(3MeBn)-Glu-pro-Asp-Af3(4SaPy2Ac)-Leu-Thr-Trp- | A | A |
| | Ser-Cys]-NH2 | | |
| 3BP-4813 | DOTA-PPAc-Gln-[Cys(3MeBn)-Glu-pro-Asp-Aph(Hsfu)-Leu-Thr-Trp-Ser- | B | C |
| | Cys]-NH2 | | |

In order to test selectivity of CAIX-binding peptides toward CAIV (SinoBliological, Cat #10472-1H081H), CAXII (SinoBliological, Cat #10617-H08H or CAXIV (SinoBliological, Cat #10458-H08H), SPR assays were performed analog to the assay described above but with a different target capture procedure. For immobilization, the carbonic anhydrases were biotinylated and capture on the SPR chip using the Biotin CAPture Kit (cytiva) according to the manufacturer instructions using HBSTE, 0.1% as Running Buffer.

Table 13 describes the protocol steps for biotinylated target capturing and assessment of the binding kinetics.

TABLE 13
SPR Protocol steps with biotinylated CAIV, CAXII, or CAXIV.

| | | Contact | | Flow | |
|---|---|---|---|---|---|
| Step | Injected solution | time | | rate | |
| Startup cycle (3x): | HBSTE, 0.1% DMSO Buffer | 60 | s | 30 | μL/min |
| Washing & surface regeneration | Regeneration solution | 5 | s | | |
| Immobilize BiotinCAP reagent | Biotin Capture Reagent | 300 | s | 2 | μL/min |
| Capture target protein | 100 nM hCAIV-bio | 300 | s | 5 | μL/min |
| | 100 nM hCAXII-bio | 600 | s | 5 | μL/min |
| | 200 nM hCAXIV-bio | 600 | s | 5 | μL/min |
| 1. Binding kinetics of test compound | Dilution no. 5 (e.g. 0.2 nM) | 120 | s | 30 | μL/min |
| 2. Binding kinetics of test compound | Dilution no. 4 (e.g. 0.8 nM) | 120 | s | 30 | μL/min |
| 3. Binding kinetics of test compound | Dilution no. 3 (e.g. 3.1 nM) | 120 | s | 30 | μL/min |
| 4. Binding kinetics of test compound | Dilution no. 2 (e.g. 12.5 nM) | 120 | s | 30 | μL/min |
| 5. Binding kinetics of test compound | Dilution no. 1 (e.g. 50 nM) | 120 | s | 30 | μL/min |
| Dissociation cycle | HBSTE, 0.1% DMSO Buffer | 1200 | s | 30 | μL/min |
| Regeneration (2x) | Regeneration solution | 60 | s | 5 | μL/min |

The results of this assay for representative compounds of the the present invention are presented in Table 14. pK_{D} category A stands for pK_{D} values >8.0, category B for pK_{D} values between 7.1 and 8.0, category C for pK_{D} values between 6.1 and 7.0, and category D for pK_{D} values <6.0. Table 14 exemplifies the specificity of compounds which bind to CAIX with high affinity (pK_{D} category A), but do not bind to the related carbonic anhydrases IV, XII and XIV (all pK_{D} category D). Moreover, these examples confirm that complexation of the DOTA cage with a metal ion does not have any influence on antitarget selectivity (3BP-4555=^{nat}Lu-3BP-4552 and 3BP-4581=^{nat}Lu-3BP-4501).

TABLE 14
Compound ID and pK_{D} category of
CAIV, CAXII and CAXIV SPR assay.

pK_{D} category (affinity)
for different CA isoforms

| Compound ID | CAIV | CAIX | CAXII | CAXIV |
|---|---|---|---|---|
| 3BP-3583 | D | A | D | D |
| 3BP-4174 | D | A | D | D |
| 3BP-4452 | D | A | D | D |
| 3BP-4501 | D | A | D | D |
| 3BP-4503 | D | A | D | D |
| 3BP-4555 | D | A | D | D |
| 3BP-4581 | D | A | D | D |

### Example 17: Plasma Stability Assay

In order to determine the stability of representative compounds of the invention in human and mouse plasma, a plasma stability assay was carried out. Such plasma stability assay measures degradation of compounds of the present invention in blood plasma. This is an important characteristic of a compound as compounds, with the exception of pro-drugs, which rapidly degrade in plasma, generally show poor in vivo efficacy. The results of the plasma stability assays show that the investigated compounds are highly stable in human and mouse plasma.

The stability is sufficient for the diagnostic, therapeutic and theragnostic use of these compounds according to the present invention.

The plasma stability samples were prepared by spiking 50 μl plasma aliquots (all K2EDTA) with 1 μl of a 0.5 mM compound stock solution in DMSO. After vortexing the samples were incubated in a Thermomixer at 37° C. for 0, 4 (6 for 3BP-3599) and 24 hours. After incubation the samples were stored on ice until further treatment. All samples were prepared in duplicates.

A suitable internal standard was added to each sample (1 μl of a 0.5 mM stock solution in DMSO). Protein precipitation was performed using two different methods depending on the compound conditions as indicated in Table 15 and Table 16.

A) 250 μl of acetonitrile containing 1% trifluoroacetic acid was added. After incubation at room temperature for 30 min the precipitate was separated by centrifugation and 150 μl of the supernatant was diluted with 150 μl of 1% aqueous formic acid.

B) 150 μl of a zinc sulphate precipitation agent containing 78% 0.1 M zinc sulphate and 22% acetonitrile was added. After incubation at room temperature for 30 min the precipitate was separated by centrifugation. To 100 μl of the supernatant 10 μl of 1% formic acid was added followed by further incubation at 60° C. for 10 min to complete the formation of the zinc chelate, if the compound contains a free DOTA moiety.

The determination of the analyte in the clean sample solutions was performed on an Agilent 1290 UHPLC system coupled to an Agilent 6530 Q-TOF mass spectrometer. The chromatographic separation was carried out on a Phenomenex BioZen XB-C18 HPLC column (50×2 mm, 1.7 μm particle size) with gradient elution using a mixture of 0.1% formic acid in water as eluent A and acetonitrile as eluent B (²% B to 41% in 7 min, 800 μl/min, 40° C.). Mass spectrometric detection was performed in positive ion ESI mode by scanning the mass range from m/z 50 to 3000 with a sampling rate of 2/sec.

From the mass spectrometric raw data the ion currents for the double or triple charged monoisotopic signal was extracted for both the compound and the internal standard.

Quantitation was performed by external matrix calibration with internal standard using the integrated analyte signals.

Additionally, recovery was determined by spiking a pure plasma sample that only contained the internal standard after treatment with a certain amount of the compound.

Carry-over was evaluated by analysis of a blank sample (20% acetonitrile) after the highest calibration sample.

The results of this assay performed on exemplary compounds of the present invention are presented in the following Table 15 and Table 16. The read-out is stated as “% intact compound remaining after 4 h, 6 h or 24 h” and means that from the amount of material at the start of the experiment the stated percentage is detected as unchanged material at the end of the experiment by LC-MS quantification. Since all compounds are more than 50% intact after at least 4 h they are considered as stable enough for diagnostic and therapeutic applications.

TABLE 15
Results of the mouse plasma stability assay.

| | Protein precipitation | % intact compound remaining | |
|---|---|---|---|
| Compound ID | method | (incubation time) | |
| 3BP-2833 | A | 92% | (4 h) |
| 3BP-2837 | A | >95% | (4 h) |
| 3BP-2949 | A | 95% | (4 h) |
| 3BP-2959 | A | >95% | (4 h) |
| 3BP-3058 | A | 95% | (4 h) |
| 3BP-3427 | A | >95% | (4 h) |
| 3BP-3432 | A | 98% | (4 h) |
| 3BP-3434 | A | >95% | (4 h) |
| 3BP-3478 | A | >95% | (4 h) |
| 3BP-3491 | A | >95% | (4 h) |
| 3BP-3503 | A | >95% | (4 h) |
| 3BP-3562 | A | 89% | (4 h) |
| 3BP-3583 | B | 79% | (24 h) |
| 3BP-3584 | A | >95% | (24 h) |
| 3BP-3599 | A | 52% | (6 h) |
| 3BP-3732 | A | >95% | (24 h) |
| 3BP-3734 | A | 79% | (24 h) |
| 3BP-3740 | A | >95% | (24 h) |
| 3BP-3840 | A | >95% | (24 h) |
| 3BP-3842 | A | >95% | (24 h) |
| 3BP-3845 | B | 76% | (24 h) |
| 3BP-3859 | A | >95% | (24 h) |
| 3BP-3870 | A | 92% | (24 h) |
| 3BP-3874 | A | >95% | (24 h) |
| 3BP-3905 | A | 83% | (24 h) |
| 3BP-3906 | A | >95% | (24 h) |
| 3BP-3934 | A | >95% | (24 h) |
| 3BP-4053 | B | 91% | (24 h) |
| 3BP-4096 | B | 93% | (24 h) |
| 3BP-4101 | B | 93% | (24 h) |
| 3BP-4103 | A | 81% | (24 h) |
| 3BP-4112 | A | >95% | (24 h) |
| 3BP-4128 | A | 87% | (24 h) |
| 3BP-4133 | A | >95% | (24 h) |
| 3BP-4142 | A | 91% | (24 h) |
| 3BP-4174 | A | >95% | (24 h) |
| 3BP-4237 | A | >95% | (24 h) |
| 3BP-4242 | A | >95% | (24 h) |
| 3BP-4253 | A | >95% | (24 h) |
| 3BP-4255 | A | 81% | (24 h) |
| 3BP-4257 | A | 94% | (24 h) |
| 3BP-4258 | A | 93% | (24 h) |
| 3BP-4264 | A | >95% | (24 h) |
| 3BP-4367 | A | >95% | (24 h) |
| 3BP-4369 | A | >95% | (24 h) |
| 3BP-4503 | A | >95% | (24 h) |
| 3BP-4555 | B | >95% | (24 h) |
| 3BP-4581 | B | >95% | (24 h) |

### Example 18: Proteolytic Stability Assay Against NEP

TABLE 16
Results of the human plasma stability assay.

| Compound | Protein precipitation | % intact compound remaining | |
|---|---|---|---|
| ID | method | after 24 h incubation | |
| 3BP-3583 | B | 71% | (24 h) |
| 3BP-4503 | A | >95% | (24 h) |
| 3BP-4555 | B | >95% | (24 h) |
| 3BP-4581 | B | >95% | (24 h) |

Peptides are often sensitive to proteolytic cleavage in the blood (Werle et al., Amino Acids, 2006, 30, 351-367). If peptides degrade rapidly, in vivo elimination could be dominated by metabolism. Since metabolites often demonstrate poor binding to the target, performance of the compound during radiopharmaceutical use (diagnostic or therapeutic) can be significantly decreased. Therefore, evaluating the stability of the compound against proteases in the early stages of compound development is essential.

NEP (neutral endopeptidase, EC 3.4.24.11, CD10, CALLA, endopeptidase 24.11, enkephalinase, neprilysin, membrane metallopeptidase A) is a membrane-bound metallopeptidase representative for membrane-bound peptidases which are mainly responsible for the activation and deactivation of bioactive peptides (Antczak et al., Bioessays, 2001, 23, 251-260). It is expressed on neutrophils and highly active in blood (Antczak et al., Bioessays, 2001, 23, 251-260). In addition, the cleavage pattern of NEP is very broad, covering many different peptide hormones, with more than 50 different natural substrates already described (Bayes-Genis et al., Journal of the American College of Cardiology, 2016, 68, 639-653). NEP preferably cleaves amide bonds between a hydrophilic and hydrophobic amino acid (preferably leucine or phenylalanine), even in small cyclic peptides such as CNP. Plamboeck et al. (2005) showed a significant impact of NEP caused cleavage on the pharmacokinetic behavior of peptides (Plamboeck et al., Diabetologia, 2005, 48, 1882-1890).

To assess the stability of compounds against NEP, the compounds were subjected to an in vitro assay based on the protocol described by Edelson et al. (Edelson et al., Pulmonary pharmacology & therapeutics, 2013, 26, 229-238).

Recombinant soluble human NEP (BioTechne, Wiesbaden, Germany) at a concentration of 100 ng/mL was mixed with the compound (10 μM) and a stable internal standard (10 μM) and incubated at 37° C. After several time points, samples were taken and analyzed with LC-MS.

The determination of the analyte in the clean sample solutions was performed on an Agilent 1290 UHPLC system coupled to an Agilent 6530 Q-TOF mass spectrometer. The chromatographic separation was carried out on a Phenomenex BioZen XB-C18 HPLC column (50×2 mm, 1.7 μm particle size) with gradient elution using a mixture of 0.1% formic acid in water as eluent A and acetonitrile as eluent B (²% B to 41% in 7 min, 800 μl/min, 40° C.). Mass spectrometric detection was performed in positive ion ESI mode by scanning the mass range from m/z 50 to 3000 with a sampling rate of 2/sec.

From the mass spectrometric raw data the ion currents for the double or triple charged monoisotopic signal was extracted for both, the compound and the internal standard.

Quantitation was performed by external matrix calibration with internal standard using the integrated analyte signals.

The activity of NEP was examined using a commercially available chromogenic substrate of NEP.

The results of this assay performed on some exemplary compounds of the present invention are given in the following Table 17. The result is stated as “% intact compound remaining after 24 h incubation” and means that from the amount of material at the start of the experiment the stated percentage is detected as intact material at the end of the experiment by LC-MS quantification. Since all compounds are more than 50% intact after 24 h they are considered as stable enough for diagnostic and therapeutic applications.

TABLE 17
Results of the NEP stability assay.

| | % intact compound remaining |
|---|---|
| Compound ID | after 24 h incubation |
| 3BP-2837 | >95% |
| 3BP-2949 | 82% |
| 3BP-2959 | >95% |
| 3BP-3058 | >95% |
| 3BP-3282 | >95% |
| 3BP-3382 | >95% |
| 3BP-3427 | >95% |
| 3BP-3432 | >95% |
| 3BP-3434 | >95% |
| 3BP-3478 | 63% |
| 3BP-3491 | >95% |
| 3BP-3503 | >95% |
| 3BP-3562 | >95% |
| 3BP-3583 | >95% |

### Example 19: ¹¹¹In- and ¹⁷⁷Lu-labeling of selected compounds

In order to serve as a diagnostically, therapeutically, or theragnostically active agent, a compound needs to be labeled with a radioactive isotope. The labeling procedure needs to be appropriate to ensure a high radiochemical yield and purity of the radiolabeled compound of the invention. This example shows that the compounds of the present invention are appropriate for radiolabeling and can be labeled in high radiochemical yield and purity.

30-150 MBq of ¹¹¹InCl₃ (in 0.02 M HCl; Curium, Germany) were mixed with 1 nmol of compound (200 μM stock solution in 0.1 M HEPES pH 7) per 30 MBq and buffer (A A: 1 M sodium acetate/ascorbic acid buffer pH 5 containing 25 mg/ml methionine or B: 1 M sodium acetate buffer pH 5 containing 25 mg/ml methionine) at a final buffer concentration of 0.1-0.2 M. The mixture was heated to 80° C. for 20-30 min. After cooling down, DTPA (Heyl, Germany) and TWEEN-20 were added at a final concentration of 0.2 mM and 0.1%, respectively (Formulation A). In some instances, 20 μL of a 200 mg/mL ascorbic acid solution (Woerwag Pharma, Germany) per 100 μL reaction mixture was additionally added at the end of synthesis (Formulation B).

0.2-2.0 GBq ¹⁷⁷LuCl₃ (in 0.04 M HCl; ITM, Germany) were mixed with 1 nmol of compound (200 μM stock solution in 0.1 M HEPES pH 7) per 45 MBq and buffer (1 M sodium acetate/ascorbic acid buffer pH 5 containing 25 mg/ml methionine) at a final buffer concentration of ˜0.4 M. The mixture was heated to 90° C. for 20 min. After cooling down, DTPA and TWEEN-20 were added at a final concentration of 0.2 mM and 0.1%, respectively.

Radiochemical purity was analyzed by HPLC. 5 μl of diluted labeling solution was analyzed with a Poroshell SB-C18 2.7 μm (Agilent). Eluent A: MeCN, eluent B: H₂O, 0.1% TFA, gradient from 5% B to 70% B within 15 min, flow rate 0.5 ml/min; detector: NaI (Raytest), DAD 230 nm. The peak eluting with the dead volume represents free radionuclide, the peak eluting with the peptide-specific retention time as determined with an unlabeled sample represents radiolabeled compound.

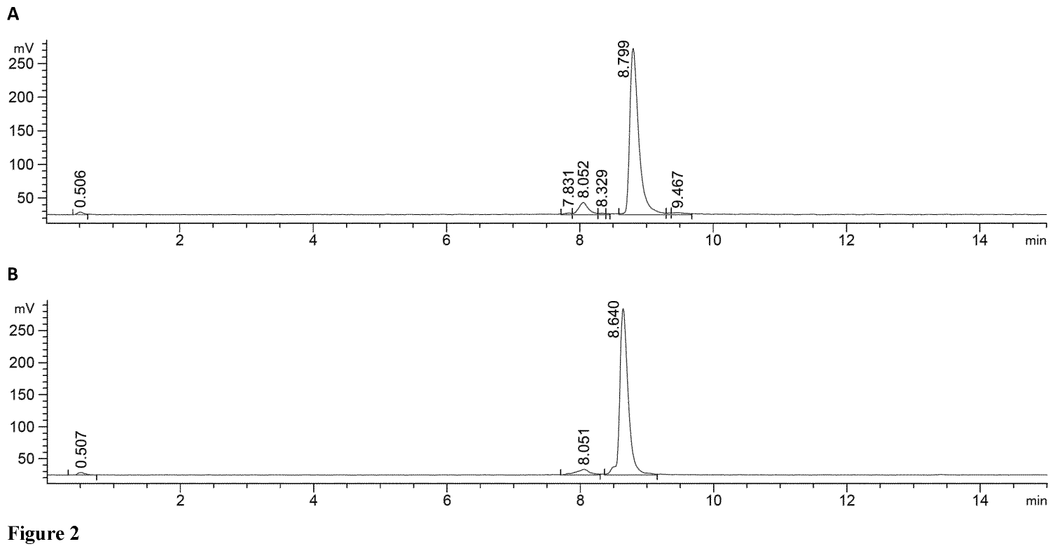

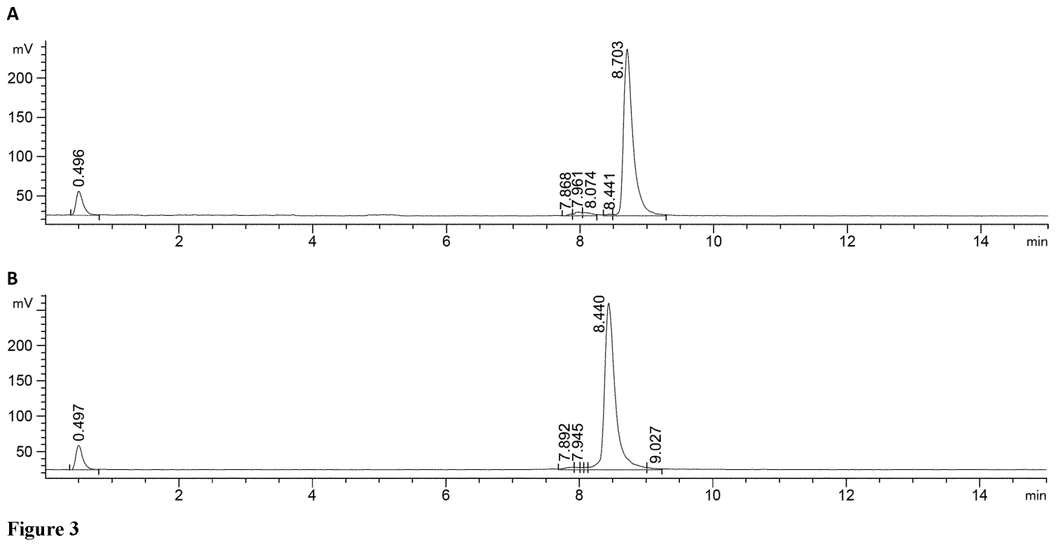

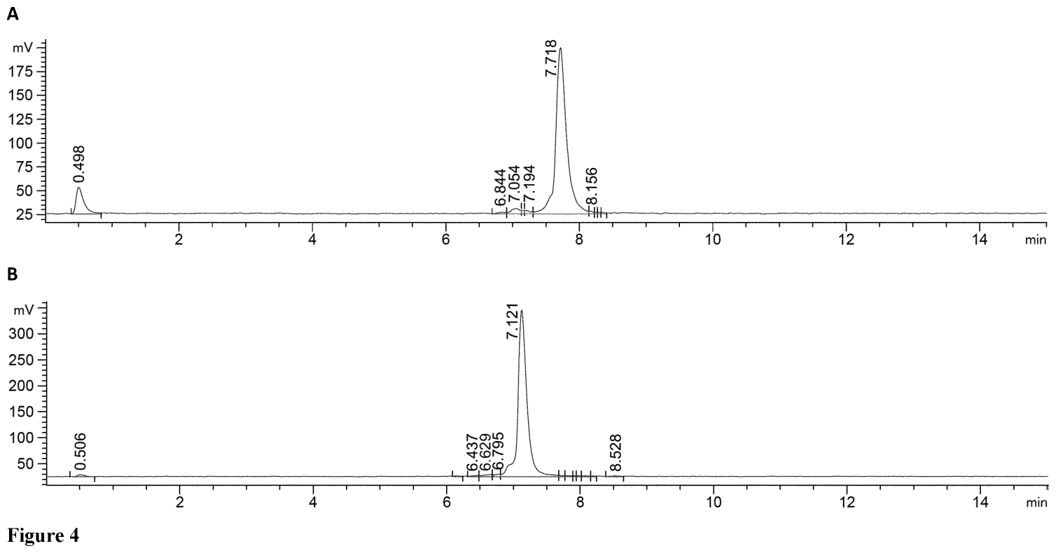

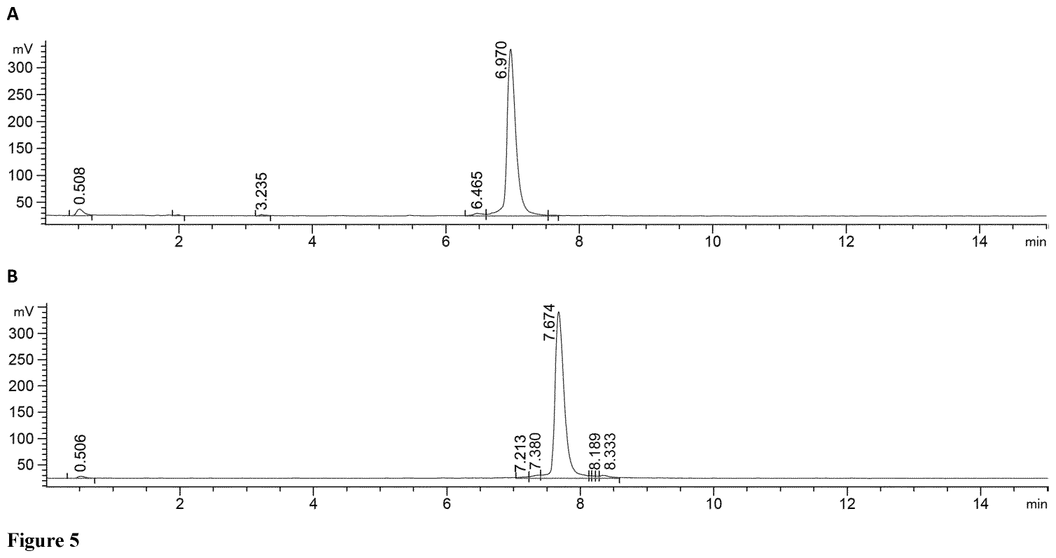

Radiochemical purity was ≥80% at end of synthesis. Exemplary radiochemical purities for selected ¹¹¹In-labeled compounds are shown in Table 18. The radiochromatograms for exemplary compounds of the invention are shown in FIGS. 2 to 5 with all peaks with an HPLC area ≥0.5% labeled with their retention times, whereby FIG. 2A shows a radiochromatogram of ¹¹¹In-3BP-3478, whereby FIG. 2B shows a radiochromatogram of ¹¹¹In-3BP-3583, whereby FIG. 3A shows a radiochromatogram of ¹¹¹In-3BP-3840, whereby FIG. 3B shows a radiochromatogram of ¹¹¹In-3BP-4175, whereby FIG. 4A shows a radiochromatogram of ¹¹¹In-3BP-4237, whereby FIG. 4B shows a radiochromatogram of ¹¹¹In-3BP-4452, whereby FIG. 5A shows a radiochromatogram of ¹¹¹In-3BP-4501, whereby FIG. 5B shows a radiochromatogram of ¹¹¹In-3BP-4503.

TABLE 18
Radiochemical purity of ¹¹¹In-labeled
compounds as measured by HPLC.

| | Labeling | | HPLC retention | HPLC |
|---|---|---|---|---|
| Compound ID | buffer | Formulation | time [min] | area % |
| ¹¹¹In-3BP-3478 | A | A | 8.8 | 87.9 |
| ¹¹¹In-3BP-3562 | A | A | 8.9 | 90.8 |
| ¹¹¹In-3BP-3583 | A | A | 8.6 | 92.9 |
| ¹¹¹In-3BP-3583 | B | B | 8.6 | 96.4 |
| ¹¹¹In-3BP-3584 | A | A | 8.7 | 89.4 |
| ¹¹¹In-3BP-3599 | A | A | 8.6 | 96.5 |
| ¹¹¹In-3BP-3734 | B | B | 8.5 | 86.3 |
| ¹¹¹In-3BP-3740 | B | B | 9.0 | 93.4 |
| ¹¹¹In-3BP-3840 | A | A | 8.7 | 86.2 |
| ¹¹¹In-3BP-3842 | A | A | 8.2 | 81.5 |
| ¹¹¹In-3BP-3859 | B | B | 8.3 | 89.1 |
| ¹¹¹In-3BP-3870 | B | B | 9.0 | 91.3 |
| ¹¹¹In-3BP-3874 | B | B | 9.1 | 91.6 |
| ¹¹¹In-3BP-3934 | B | B | 8.4 | 85.0 |
| ¹¹¹In-3BP-4096 | B | B | 8.2 | 92.7 |
| ¹¹¹In-3BP-4101 | B | B | 8.5 | 93.2 |
| ¹¹¹In-3BP-4103 | B | B | 8.6 | 93.9 |
| ¹¹¹In-3BP-4112 | B | B | 8.3 | 95.0 |
| ¹¹¹In-3BP-4128 | B | B | 10.5 | 86.6 |
| ¹¹¹In-3BP-4133 | B | B | 9.5 | 90.4 |
| ¹¹¹In-3BP-4142 | B | B | 8.8 | 96.2 |
| ¹¹¹In-3BP-4144 | B | B | 9.1 | 94.1 |
| ¹¹¹In-3BP-4174 | B | B | 9.0 | 82.6 |
| ¹¹¹In-3BP-4175 | B | B | 8.4 | 88.2 |
| ¹¹¹In-3BP-4176 | B | B | 9.4 | 80.2 |
| ¹¹¹In-3BP-4237 | B | B | 7.7 | 84.6 |
| ¹¹¹In-3BP-4242 | B | B | 7.8 | 89.0 |
| ¹¹¹In-3BP-4253 | B | B | 8.5 | 93.2 |
| ¹¹¹In-3BP-4255 | B | B | 8.2 | 94.4 |
| ¹¹¹In-3BP-4257 | B | B | 8.2 | 89.3 |
| ¹¹¹In-3BP-4261 | B | B | 8.3 | 82.4 |
| ¹¹¹In-3BP-4264 | B | B | 8.7 | 84.0 |
| ¹¹¹In-3BP-4324 | B | B | 7.4 | 93.0 |
| ¹¹¹In-3BP-4367 | B | B | 7.8 | 94.6 |
| ¹¹¹In-3BP-4369 | B | B | 7.7 | 94.9 |
| ¹¹¹In-3BP-4400 | B | B | 7.5 | 91.8 |
| ¹¹¹In-3BP-4405 | B | B | 8.2 | 90.9 |
| ¹¹¹In-3BP-4413 | B | B | 8.3 | 95.8 |
| ¹¹¹In-3BP-4445 | B | B | 8.0 | 91.2 |
| ¹¹¹In-3BP-4448 | B | B | 7.4 | 96.1 |
| ¹¹¹In-3BP-4452 | B | B | 7.1 | 93.2 |
| ¹¹¹In-3BP-4453 | B | B | 7.1 | 89.3 |
| ¹¹¹In-3BP-4455 | B | B | 7.5 | 94.6 |
| ¹¹¹In-3BP-4489 | B | B | 7.3 | 88.1 |
| ¹¹¹In-3BP-4500 | B | B | 6.9 | 88.0 |
| ¹¹¹In-3BP-4501 | B | B | 7.0 | 94.0 |
| ¹¹¹In-3BP-4503 | B | B | 7.7 | 93.4 |
| ¹¹¹In-3BP-4504 | B | B | 7.4 | 91.3 |
| ¹¹¹In-3BP-4505 | B | B | 7.5 | 86.1 |

### Example 20: Radioligand Binding Assay

In order to assess the affinity of ¹¹¹In-labeled compounds to CAIX of different species, a radioligand binding assay was established.

CHO-VGT cells were transfected with the human, dog, and mouse CAIX (InSCREENex GmbH) and cultured in Ham's F-12 medium (Sigma-Aldrich #N4888) supplemented with 10% fetal bovine serum (FCS), 2 mM L-glutamine, 100 U/mL Penicillin, and 0.1 mg/mL streptomycin. Cells were detached with Accutase (Biolegend #423201) and counted using a particle counter (CASY Model TT; Scharfe Systems, Germany). Cell concentrations were adjusted to 3×10⁵ mL⁻¹, and 1.000 μL of the suspension per well were dispensed into flat-clear-bottom 24 well plates.

Approximately 24 hours after re-seeding, the medium was aspirated and the cells were washed once with 1000 μL assay medium (Ham's medium without additives). To determine total binding, 700 μL of assay medium and 100 μL of the radioligand dilutions were added to the wells in triplicates. To determine non-specific binding, 600 μL of binding medium, 100 μL of the radioligand dilutions and 100 μL of a blocking solution containing 8 μM non-labeled compound were added to the wells in triplicates. At the end of incubation time (8 h at 37° C.), cells were washed and subsequently harvested in 300 μL RIPA buffer (Thermo Scientific #89901) containing Protease Inhibitor Cocktail Set I (Calbiochem #539131). Radioactivity of the cell lysate of each well was counted using a gamma counter (1470 Wizard, PerkinElmer, USA). An aliquot of each radioligand dilution was included in the gamma counter measurements to allow for determination of their actual concentrations via their known specific activity.

The results of the radioligand binding assay are shown in Table 19. pK_{D} category A stands for pK_{D} values >8.0, category B for pK_{D} values between 7.1 and 8.0, category C for pK_{D} values between 6.1 and 7.0, and category D for pK_{D} values ≤6.0.

TABLE 19
Results of the radioligand binding assay.

| Compound ID | Species | pK_{D} category (activity) |
|---|---|---|
| 3BP-3583 | Human | A |
| 3BP-3583 | Dog | A |
| 3BP-3583 | Mouse | C |
| 3BP-4452 | Human | A |
| 3BP-4452 | Dog | A |
| 3BP-4452 | Mouse | B |
| 3BP-4501 | Human | A |
| 3BP-4501 | Dog | A |
| 3BP-4501 | Mouse | B |
| 3BP-4503 | Human | A |
| 3BP-4503 | Dog | A |
| 3BP-4503 | Mouse | A |

### Example 21: Imaging Studies

Radioactively labeled compounds can be detected by imaging methods such as SPECT and PET. Furthermore, the data acquired by such techniques can be confirmed by direct measurement of radioactivity contained in the individual organs prepared from an animal injected with a radioactively labeled compound of the invention. Thus, the biodistribution (the measurement of radioactivity in individual organs) of a radioactively labeled compound can be determined and analyzed. This example shows that the compounds of the present invention show a biodistribution appropriate for diagnostic imaging and therapy of tumors.

All animal experiments were conducted in compliance with the German animal protection laws. Female swiss nude mice (6- to 8-week-old, Charles River Laboratories, France) were inoculated with 5×10⁶ target-positive HT-29 (DSMZ, RRID: CVCL_0320) or SK-RC-52 cells (MSKCC, RRID: CVCL_6198 in one shoulder. In some cases, both cell lines were inoculated in opposite shoulders in the same mouse. When tumors reached a size of >150 mm³ mice received ˜30 MBq ¹¹¹In-labelled compounds of the invention (diluted to 100 μL with PBS) administered intravenously via the tail vein. Images were obtained on a NanoSPECT/CT system (Mediso Medical Imaging Systems, Hungary) using exemplarily the following acquisition and reconstruction parameters (Table 20).

TABLE 20
Acquisition and reconstruction parameters
of NanoSPECT/CT imaging.
Acquistion parameters SPECT

| System | NanoSPECT/CT ™ | |
|---|---|---|
| Scan range | whole body, 3-bed holder (mouse hotel) | |
| Time per projection | 60 | s |

| | |
|---|---|
| Aperture model, | Aperture #2, 1.5 mm |
| pinhole diameter | |

Reconstruction parameters

| | |
|---|---|
| Method | HiSPECT (Scivis), iterative reconstruction |
| Smoothing | 35% |
| Iterations | 9 |
| Voxel size | 0.15 mm × 0.15 mm × 0.15 mm |

Acquisition parameters CT

| System | NanoSPECT/CT ™ | |
|---|---|---|
| Scan range | whole body, 3-bed holder (mouse hotel) | |
| Scan duration | 7 | minutes |
| Tube voltage | 45 | kVp |
| Exposure time | 500 | ms |

| | |
|---|---|
| Number of projections | 240 |

Imaging data were saved as DICOM files and analysed using VivoQuant™ software (InviCRO, USA). Results are expressed as a percentage of injected dose per gram of tissue (% ID/g). Two animals were used per time point.

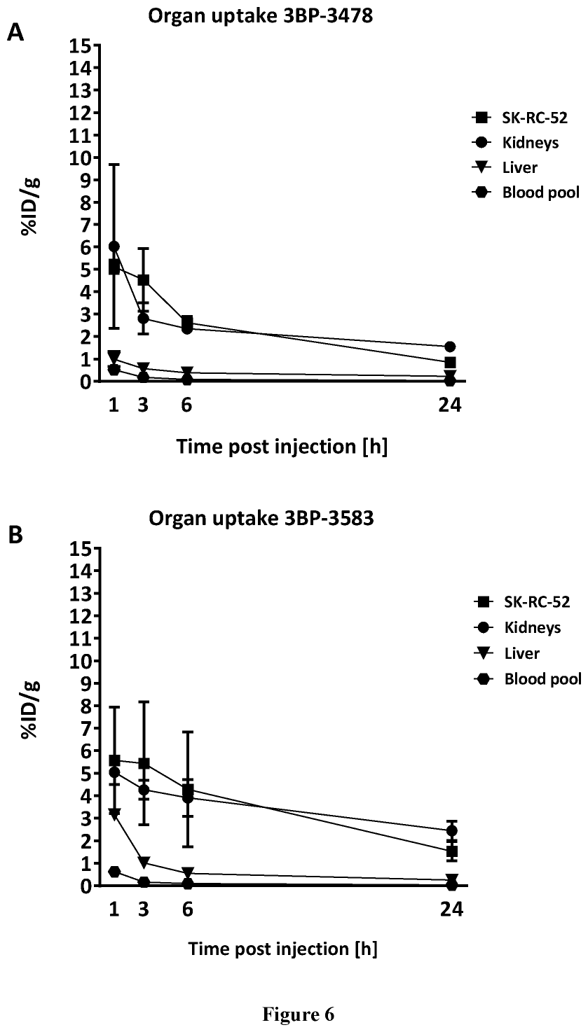

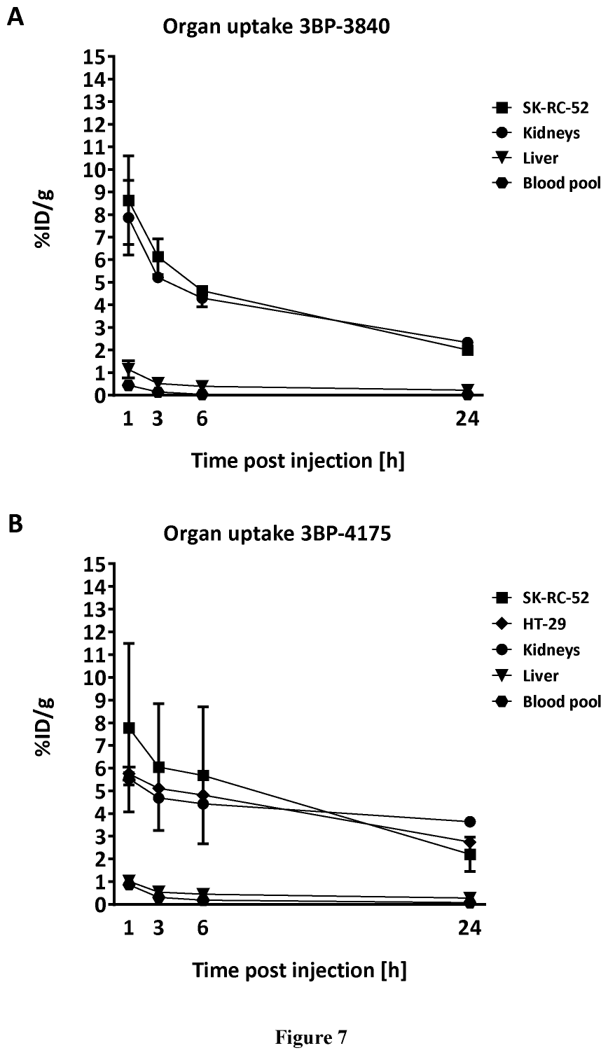

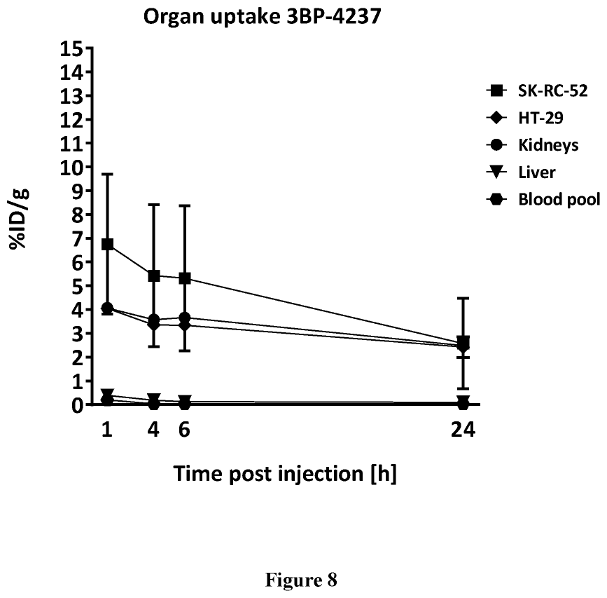

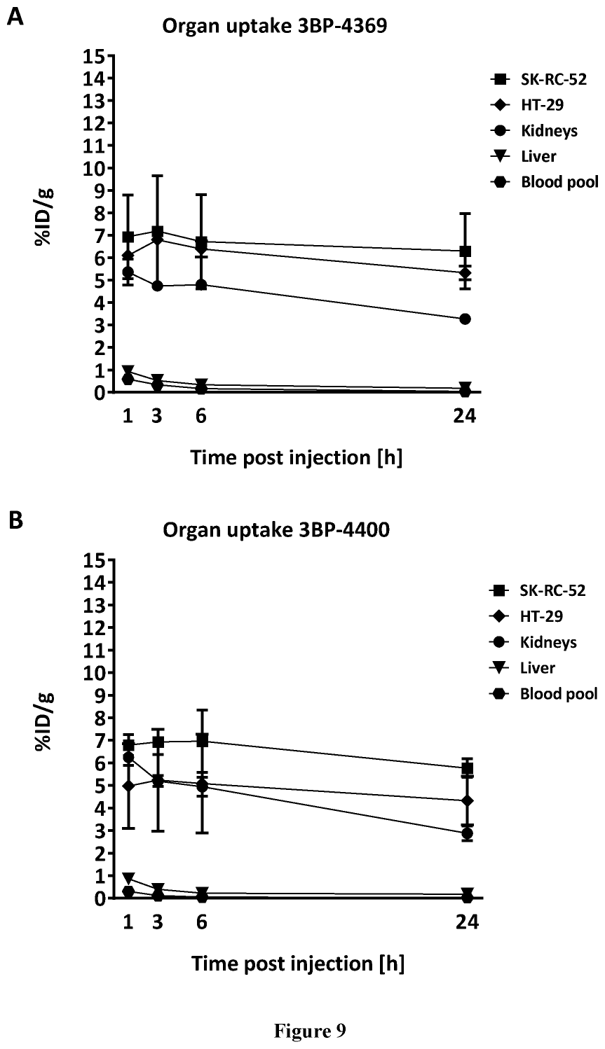

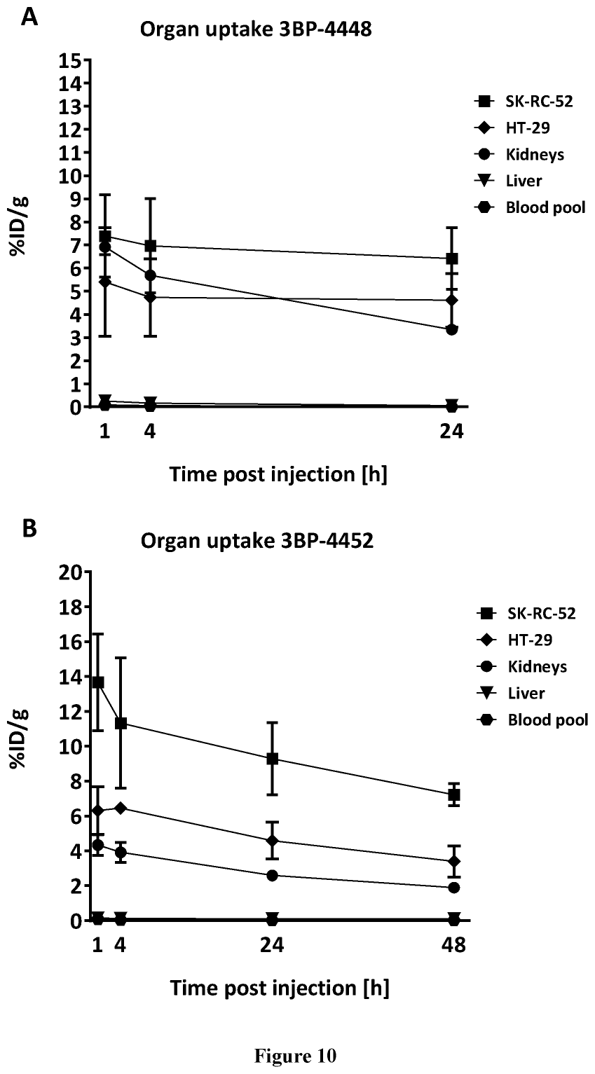

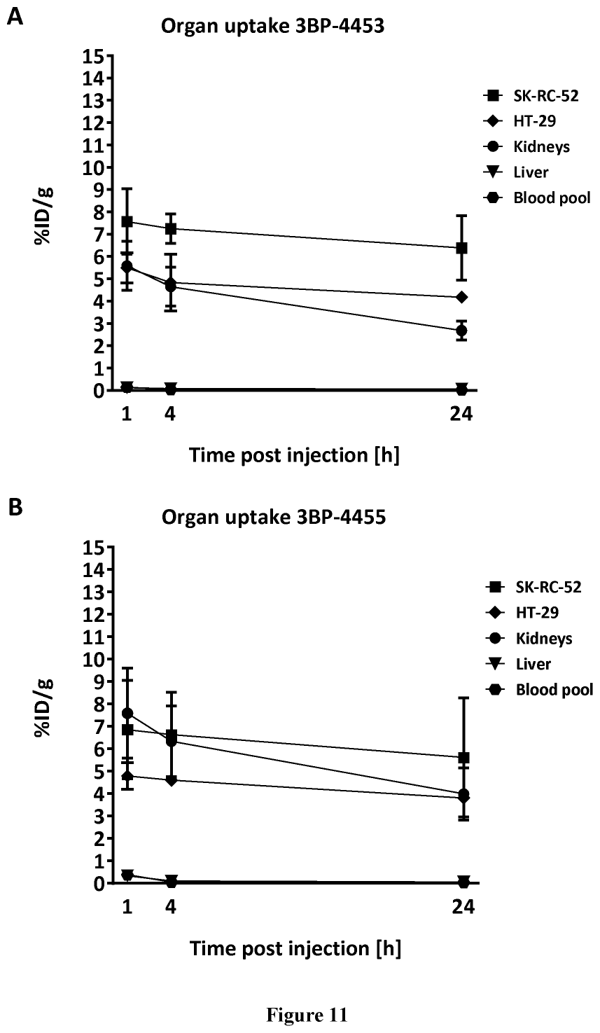

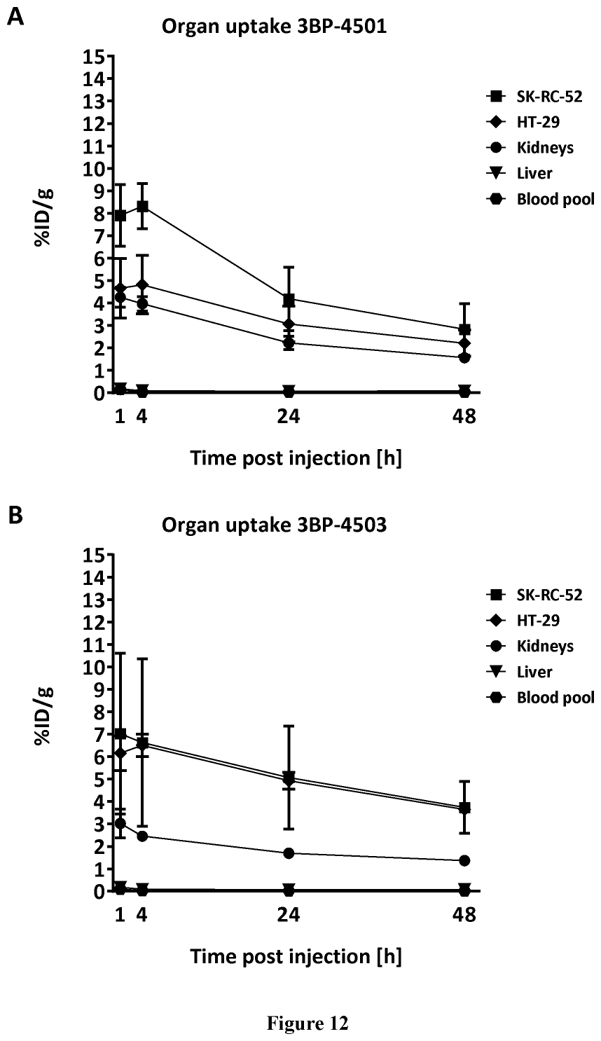

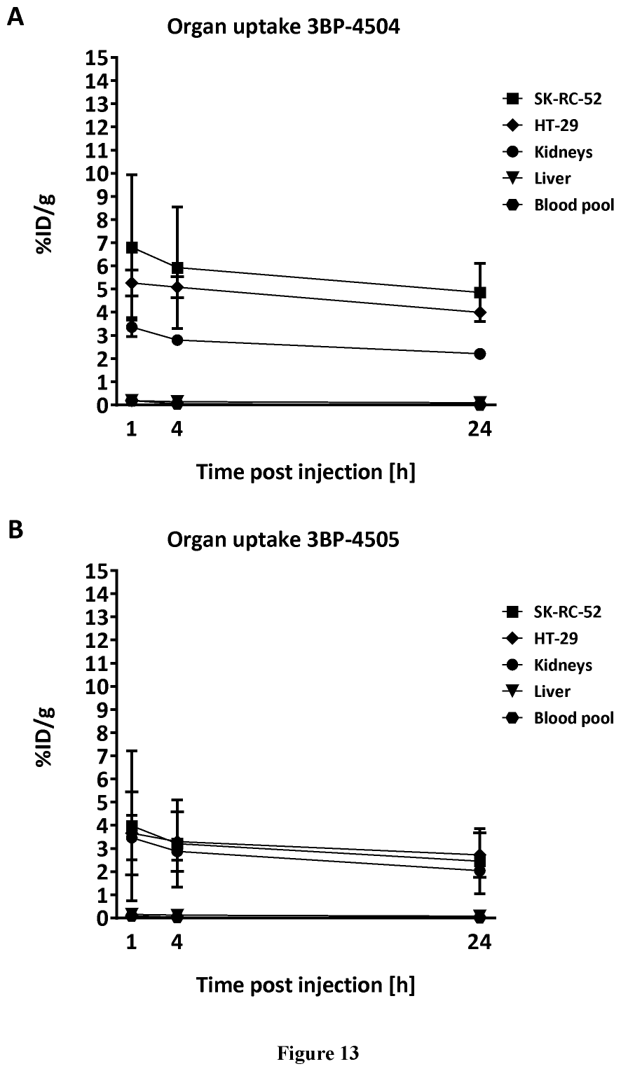

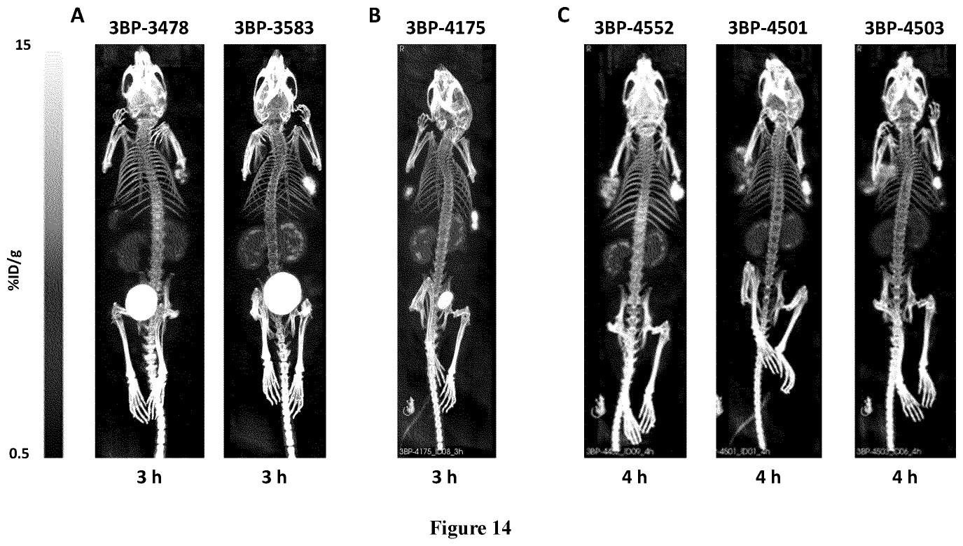

The results of the imaging studies for exemplary compounds of the invention are shown in FIGS. 6-14, demonstrating a mean peak tumor uptake of ≥4.0% ID/g and up to 13.7% ID/g for those exemplary compounds.

### Example 22: Prevalence of CAIX Expression in CRC, PDAC, Sq. NSCLC, SCCHN, TNBC and ccRCC Cancers

CAIX protein expression was assessed using a validated immunohistochemistry assay (IHC) with an anti-CAIX antibody (M75) on a panel of 30 ccRCC, 70 PDAC, 80 Sq. NSCLC, 60 SCCHN, 95 TNBC and 85 CRC tumor specimens as well as healthy tissue. H-score was calculated for each individual sample.

The IHC method was adapted from Rasheed S. et al. (Pathol Res Pract. 2009, 205(1), 1-9). Tissue Microarrays (TMAs) containing colon carcinoma specimens (#BC000110), healthy normal colon tissue (#C0727), normal lung tissue (#LCN241), lung SCC (#LC808b), mixed pancreatic tissues (#PA482, #PA805c), a breast cancer (#BR1901), a head and neck cancer (#HN601d), a normal multi-organ (#FDA999w), ccRCC specimens and non-tumoral adjacent kidney tissue (#KD601a) panel were purchased from US Biomax and used for validation.

The CA9 (mouse clone M75) assay was evaluated on a semi-quantitative scale, and the percentage of tumor cells or normal cells staining at each of the following four levels was recorded: 0 (no staining), 1+ (weak staining), 2+ (moderate staining) and 3+ (strong staining). A tumor or normal sample was considered positive if at least 1% of cells demonstrated positive expression. The subcellular localization (SCL) of staining was noted for positive samples.

### Pathologist Tumor H-Score

The Pathologist H-Score was calculated based on the summation of the product of percent of cells stained at each staining intensity using the following equation: (3×% cells staining at 3+)+(2×% cells staining at 2+)+(1×% cells staining at 1+).

The measured CAIX prevalence is shown in the table below with respect to each tumor type.

TABLE 21
Prevalence of CAIX expression in CRC, Sq.
NSCLC, PDAC, SCCHN, TNBC and ccRCC cancers

| | H-Score | | | Sq. | | | |
|---|---|---|---|---|---|---|---|
| Tissue | (max = 300) | CRC | ccRCC | NSCLC | PDAC | SCCHN | TNBC |
| Malignant | >150 | 29% | 83% | 19% | 40% | 23% | 19% |
| | | (25/85) | (25/30) | (15/79) | (26/65) | (14/60) | (10/54) |
| | >100 | 35% | 83% | 20% | 51% | 33% | 24% |
| | | (30/85) | (25/30) | (16/79) | (33/65) | (20/60) | (10/54) |
| | >40 | 56% | 87% | 42% | 60% | 62% | 37% |
| | | (48/85) | (26/30) | (33/79) | (39/65) | (37/60) | (10/54) |
| Healthy | >40 | 0% | 0% | 0% | 0% | n.d. | n.d. |
| control | | (0/21) | (0/30) | (0/22) | (0/30) | | |

### Example 23: In vitro binding assay of DPI-4452, ^{nat}Lu-DPI-4452 and ^{nat}Ga-DPI-4452 to CAIX

The affinity and the kinetics of DPI-4452 (also referred to in the present application as “3BP-4452”) binding to CAIX was evaluated in a cell-free assay using Surface Plasmon Resonance (SPR) approach. Human Fc-recombinant protein was captured on the sensor chip and DPI-4452 or ^{nat}Lu-DPI-4452 or ^{nat}Ga-DPI-4452 at different concentration were injected into the system and the association and dissociation of the molecules to the target were determined (Table 22).

TABLE 22
Affinity and kinetic binding properties of
DPI-4452 for human CAIX measured by SPR

| Compound | K_{D} (nM) * | t_{1/2} (min) |
|---|---|---|
| DPI-4452 | 0.25 | 99 |
| ^{nat}Lu-DPI-4452 | 0.16 | 123 |
| ^{nat}Ga-DPI-4452 | 0.2 | 112 |

* K_{D} = equilibrium dissociation constant, min = minutes, t_{1/2} = half-life

DPI-4452, ^{nat}Lu-DPI-4452 and ^{nat}Ga-DPI-4452 compounds bind to CAIX with subnanomolar affinity and show slow dissociation kinetics. The mean dissociation half-life of the test compounds was 99 min for DPI-4452, 123 min for ^{nat}Lu-DPI-4452 and 112 min for ^{nat}Ga-DPI-4452. These results demonstrated that labeling of DPI-4452 with lutetium or gallium does not impact its affinity and kinetic properties for CAIX binding.

### Materials and Methods

Surface Plasmon Resonance (SPR) studies were performed on a Biacore™ T200 using a ligand capture approach. Basically, anti-human Fc antibodies were immobilized on the surface of a sensor chip and CAIX was captured on the functionalized surface via its Fc Tag.

Then, each compound was injected at increasing concentrations on the captured CAIX in order to measure the in real-time interaction of the compound to its target (i.e. the captured CAIX). The in real-time monitoring of the association and dissociation of the interaction gives access to the interaction kinetics parameters (i.e. the association and dissociation rate constants and the resulting affinity constants).

For each interaction assay, the background was measured on a reference flow-cell with no captured CAIX and was subtracted to the signal measured on the active flow-cell surface. Moreover, the baseline drift was corrected by performing an entire interaction cycle with the injection of running buffer instead of the compound on the active flow-cell surface (double referencing).

### Immobilization of the Human Fc-Capture Antibody Via Amine Coupling:

A series S CM5 sensor chip was used (Cytiva).
All reagents used for the immobilization procedure were part of the Amine Coupling Kit, type 2 and/or the Human Antibody Capture Kit (Cytiva). The running buffer (HBS-EP+) was prepared by 10-fold dilution of the stock HBS-EP+ 10× solution (Cytiva).
Anti-human Fc antibodies stock solution (0.5 mg/mL in 0.15 M NaCl) was diluted in 10 mM sodium acetate pH 5.0 to a final concentration of 25 μg/mL directly into a plastic vial 7 mm.
Amine coupling reagents were transferred into plastic vials 7 mm as follow:
   95 μL of 0.4 M 1-ethyl-3-(3-dimethylaminopropyl)carbodiimide (EDC).
   95 μL of 0.1 M N-hydroxysuccinimide (NHS).
   An empty plastic vial 7 mm was added in the Biacore™ sample rack for mixing.
   150 μL of 1 M ethamolamine-HCl, pH 8.5.
Plastic vials were closed with rubber caps and the sample rack was inserted into the Biacore™ T200 sample compartment.
The Biacore™ T200 Amine Wizard was used to immobilize the anti-Human Fc antibodies on the four flow-cells of a series S CM5 sensor chip simultaneously with defined contact time of 360 seconds and flow-rate of 5 μL/min.
The immobilization procedure was performed at 25° C., following Cytiva's recommendations, as described in Table 23.

TABLE 23
Immobilization procedure of the anti-human Fc antibodies
on the series S CM5 sensorchip surface

| | | Contact | Flow-rate | Injected |
|---|---|---|---|---|
| Step | Injected solution | time (s) | (μL/min) | Volume |
| Surface activation | EDC/NHS | 420 | 10 | 70 μL |
| Ligand immobilization | Anti-human Fc | 360 | 5 | 30 μL |
| | antibodies at 25 | | | |
| | μg/mL | | | |
| Surface deactivation | 1M ethanolamine | 420 | 10 | 70 μL |

This procedure allowed to reach immobilization levels between 6000 and 9000 RU of anti-human Fc antibodies on the sensor chip surface.

### Preparation of the Human CAIX Solution:

CAIX (Sino Biological, 10107-H02H) stock solution was prepared from lyophilized material reconstituted with deionized water to a concentration of 0.25 μg/mL (3.69 μM), following the supplier recommendations.
CAIX working solution was obtained after dilution of CAIX stock solution with PBS-P+1×, 0.1% DMSO to a final concentration of 200 nM directly in a Biacore™ plastic vial.

### Preparation of the Compound Dilutions:

Preparation of intermediate 1 mM compound solutions:
   A stock solution was prepared by dissolving each compound in a respective volume of 0.1 M sodium acetate, pH 5.0 to obtain a final concentration of 1 mg/mL (474.74 μM for DPI-4452, 438.92 μM for ^{nat}Lu-DPI-4452 and 460.18 μM for ^{nat}Ga-DPI-4452).
   1 mL of intermediate 1 mM compounds solutions were prepared as follows:
      DPI-4452: 2.1 μL of stock solution was diluted in 997.9 μL of PBS-P+1×, 0.1% DMSO.
      ^{nat}Lu-DPI-4452: 2.3 μL of stock solution was diluted in 997.7 μL of PBS-P+1×, 0.1% DMSO.
      ^{nat}Ga-DPI-4452: 2.2 μL of stock solution was diluted in 997.8 μL of PBS-P+ 1×, 0.1% DMSO.
Preparation of the compounds working solutions:
   For each compound, working solutions were prepared by serial dilutions directly in the 96-well microplate as follows:
      The 50 nM working solution was prepared by dilution of 10 μL of the 1 mM intermediate dilution in 190 μL of PBS-P+1×, 0.1% DMSO.
      The 12.5 nM working solution was prepared by dilution of 50 μL of the 50 nM intermediate dilution in 150 μL of PBS-P+1×, 0.1% DMSO.
      The 3.125 nM working solution was prepared by dilution of 50 μL of the 12.5 nM intermediate dilution in 150 μL of PBS-P+1×, 0.1% DMSO.
      The 0.78 nM working solution was prepared by dilution of 50 μL of the 3.125 nM intermediate dilution in 150 μL of PBS-P+1×, 0.1% DMSO.
      The 0.19 nM working solution was prepared by dilution of 50 μL of the 0.78 nM intermediate dilution in 150 μL of PBS-P+1×, 0.1% DMSO.

### Kinetics and Affinity Constants Determination:

All closed plastic vials and the sealed 96-well microplate were placed into the sample rack and inserted into the Biacore™ T200 sample compartment.
The Biacore™ fluidic system was primed with PBS-P+1×, 0.1% DMSO and three startup cycles were performed to ensure the correct conditioning of the system before measurements.
Three different CAIX capture levels were evaluated of flow-cell 2 (˜1640 RU), flow-cell 3 (˜1150 RU) and flow-cell 4 (˜670 RU) in parallel.
A reference flow-cell (flow-cell 1) was used on which anti-human Fc antibodies were immobilized with the same density than on the active flow-cells (flow-cells 2, 3 and 4) but without the CAIX capture step.
Kinetics measurements were performed in Single Cycle Kinetics (SCK) mode at 25° C., in triplicates.
Before each measurement cycle, a blank cycle was performed by injecting running buffer (PBS-P+1×, 0.1% DMSO) instead of the compound solution through the active flow-cells.
3 M magnesium chloride (Cytiva, part of the human antibody capture kit) was injected at the end of each cycle in order to regenerate the surface.
The binding kinetics measurements procedure is detailed in Table 24.

TABLE 24
Binding kinetics measurements procedure.

| | | | Flow- | |
|---|---|---|---|---|
| | | Contact | rate | |
| Step | Injected solution | time (s) | (μL/min) | Flow-cell |
| Capture 1 | 200 nM CAIX | 200 | 5 | 2 |
| Capture 2 | 200 nM CAIX | 100 | 5 | 3 |
| Capture 3 | 200 nM CAIX | 50 | 5 | 4 |
| Compound concentration 1 | 0.19 nM dilution of compound | 120 | 30 | 1, 2, 3 and 4 |
| Compound concentration 2 | 0.78 nM dilution of compound | 120 | 30 | 1, 2, 3 and 4 |
| Compound concentration 3 | 3.125 nM dilution of compound | 120 | 30 | 1, 2, 3 and 4 |
| Compound concentration 4 | 12.5 nM dilution of compound | 120 | 30 | 1, 2, 3 and 4 |
| Compound concentration 5 | 50 nM dilution of compound | 120 | 30 | 1, 2, 3 and 4 |
| Dissociation phase | PBS-P + 1X, 0.1% DMSO | 1200 | 30 | 1, 2, 3 and 4 |
| Regeneration | 3M magnesium chloride | 120 | 20 | 1, 2, 3 and 4 |
| Stabilization phase | PBS-P + 1X, 0.1% DMSO | 120 | 20 | 1, 2, 3 and 4 |

### Data Processing and Statistical Analysis:

For each measurement, the raw surface plasmon resonance response (RU) was plotted against time (s) as a sensorgram using the Biacore™ T200 Control Software.
Raw data were corrected by double referencing with subtraction of the signal of both the reference flow-cell and the blank cycle.
The association and dissociation rate constants (kₐ and k_{d}, respectively), as well as the dissociation constant K_{D} and dissociation half-life t_{1/2} were determined from the corrected sensorgrams by fitting the data to a 1:1 Langmuir binding model with the Biacore™ T200 evaluation software.

### Example 24: In Vivo Efficacy of ¹⁷⁷Lu-DPI-4452 in HT-29 (CRC) and SK-RC-52 (ccRCC) Human Cancer Cell Line Xenograft Mouse Models

The human colorectal cancer cell line HT-29 was cultured in Modified McCoy's 5a Medium supplemented with 10% FBS+1% Pen/Strep, and the human clear cell renal cancer cell line SK-RC-52 was cultured in RPMI-1640 GlutaMax-I supplemented with 10% FBS+1% Pen/Strep.

2×10⁶ cells were suspended in 100 μL PBS and Matrigel (1:1) and subcutaneously implanted into the neck of anesthetized female immunodeficient NMRI nude mice. Tumor volume (0.52×(length×width²)) and animal weight was monitored twice weekly until 42 days post treatment initiation. Animals were humanely euthanized by cervical dislocation at predefined study or humane endpoints.

Animals were randomized into equal groups based on tumor volume and body weight. Treatments were initiated at a mean group tumor volume of 140-180 mm³ and administered intravenously in the tail vein in a 100 μL dosing volume.

For both models, treatment groups consisted of 10 mice per group and received either a A) Single administration (day 1) of vehicle, B) Single administration (day 1) of 100 MBq of ¹⁷⁷Lu-DPI-4452, C) Single administration (day 1) of 33 MBq of ¹⁷⁷Lu-DPI-4452 or D) Three administrations (day 1, 8, 15) of 33 MBq of ¹⁷⁷Lu-DPI-4452. To correlate the ⁶⁸Ga-DPI-4452 signal with the ¹⁷⁷Lu-DPI-4452 uptake in tumors, an additional satellite group E of 6 mice received a single administration (day 1) of 10 MBq of ⁶⁸Ga-DPI-4452 followed by a single administration (day 8) of 33 MBq of ¹⁷⁷Lu-DPI-4452.

In groups A-D, radioactivity uptake (as % of injected dose/gram tissue) was assessed in the tumor, kidney, and liver in 3 animals per treatment group by whole-body SPECT/CT imaging (nanoScan SPECT/CT, Mediso) at 4 h after each ¹⁷⁷Lu-DPI-4452 administration, using the following parameters:
Imaging bed: Two/three mice bed
CT acquisition
   Helical scan, 480 projections
   Pitch=1.0
   Voltage=50 kVp
   Exposure=170 ms
SPECT acquisition:
   Pinhole SPECT
   Energy windows: Primor Peak 208 full width: 20%, Secondary 112.9, Tertiary: 56.1 keV
   Acquisition time: Up to 30 min
   Frame time: 30 s

In the satellite mice (group E), radioactivity uptake (as % of injected dose/gram tissue) was assessed in the tumor, kidney, and liver in all 6 animals by both whole-body SPECT imaging at 4 h after administration of 33 MBq ¹⁷⁷Lu-DPI-4452, and by whole-body PET/CT imaging (nanoScan PET/CT, Mediso) at 1 h after 10 MBq ⁶⁸Ga-DPI-4452 administration, using the following parameters:
Animals per bed: 3
PET acquisition time: Up to 10 min
PET acquisition start: 1 hour after injection

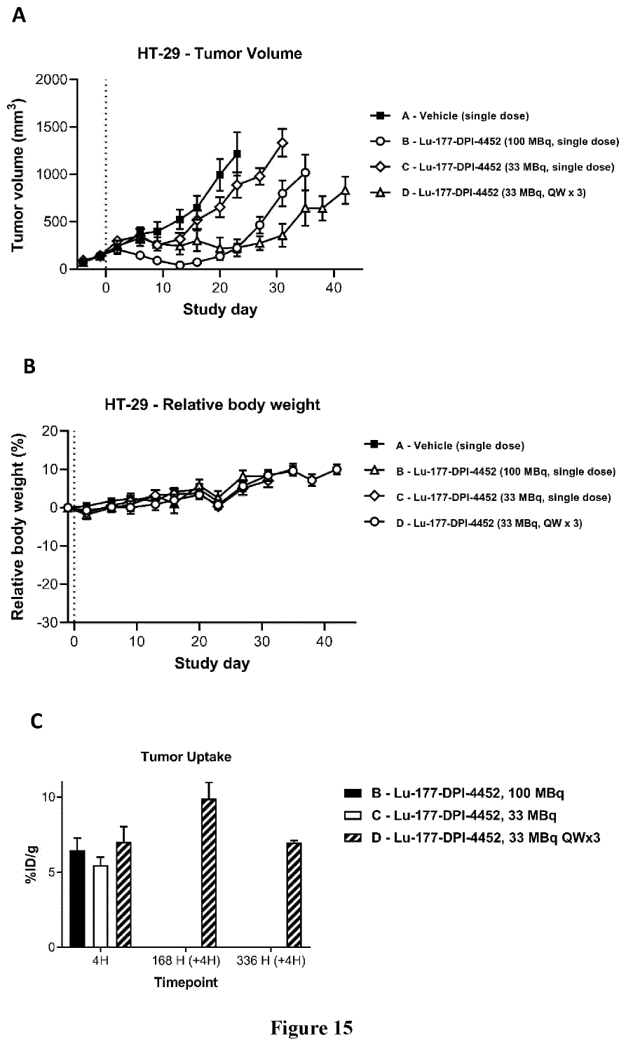

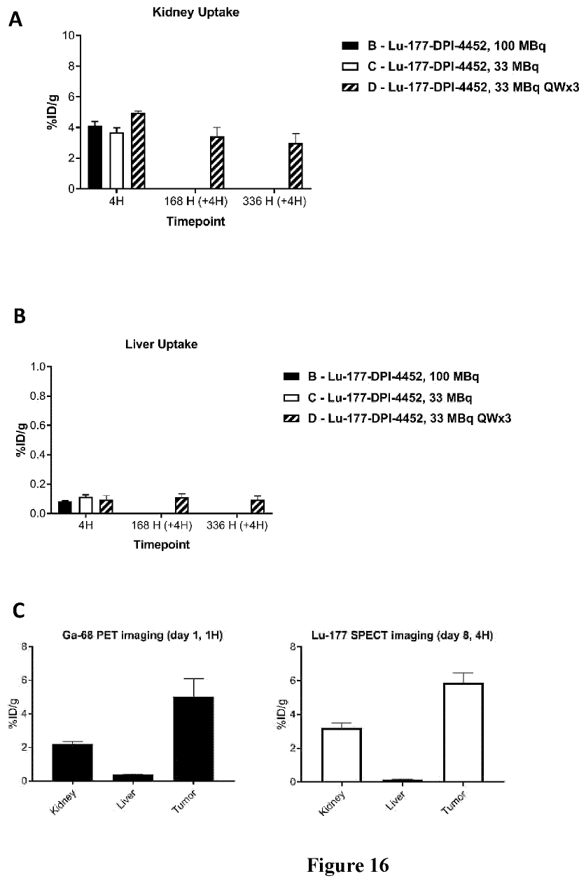

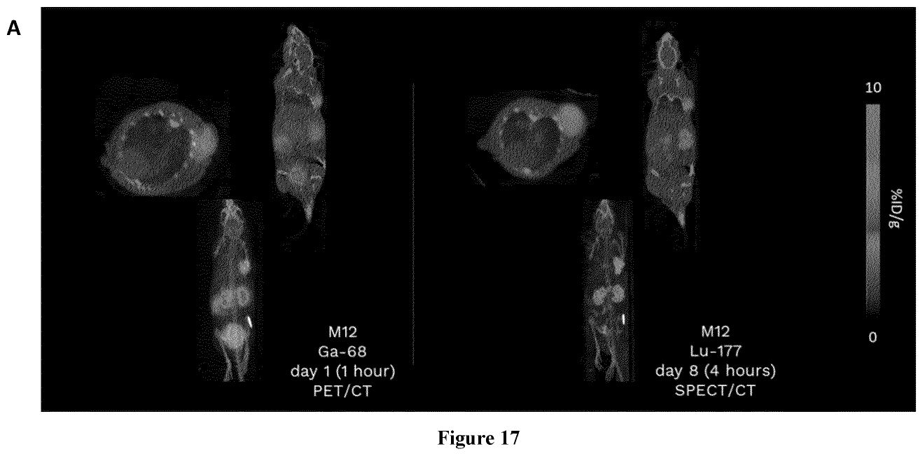

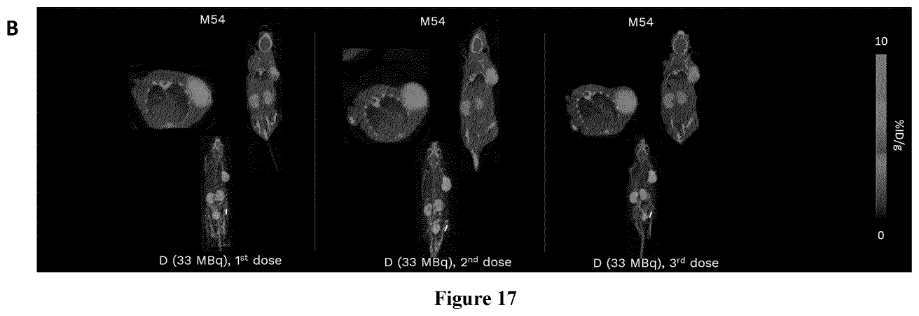

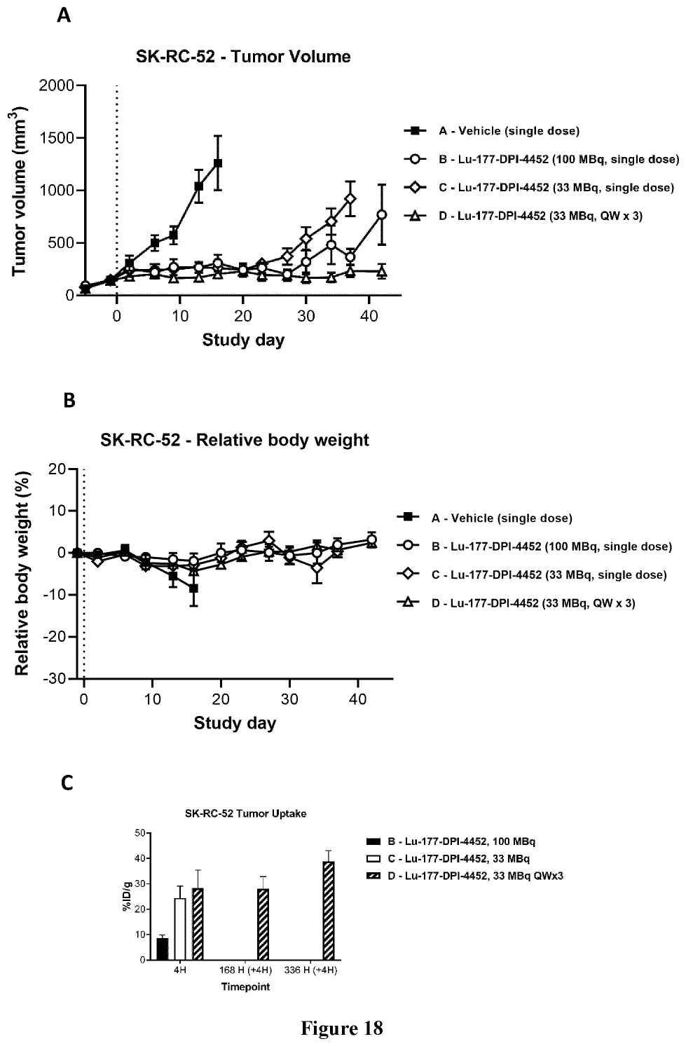

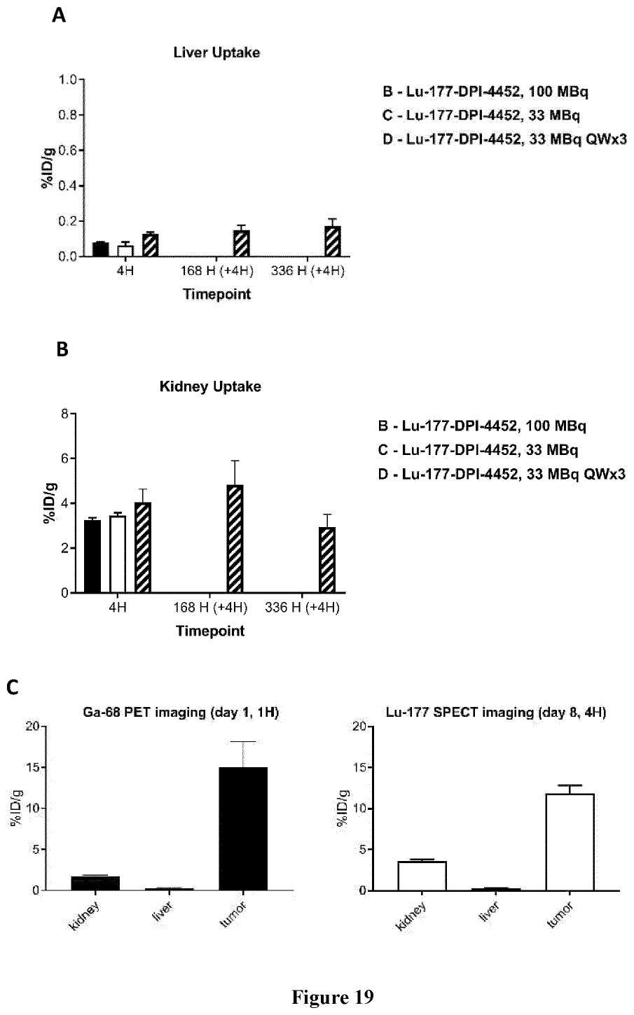

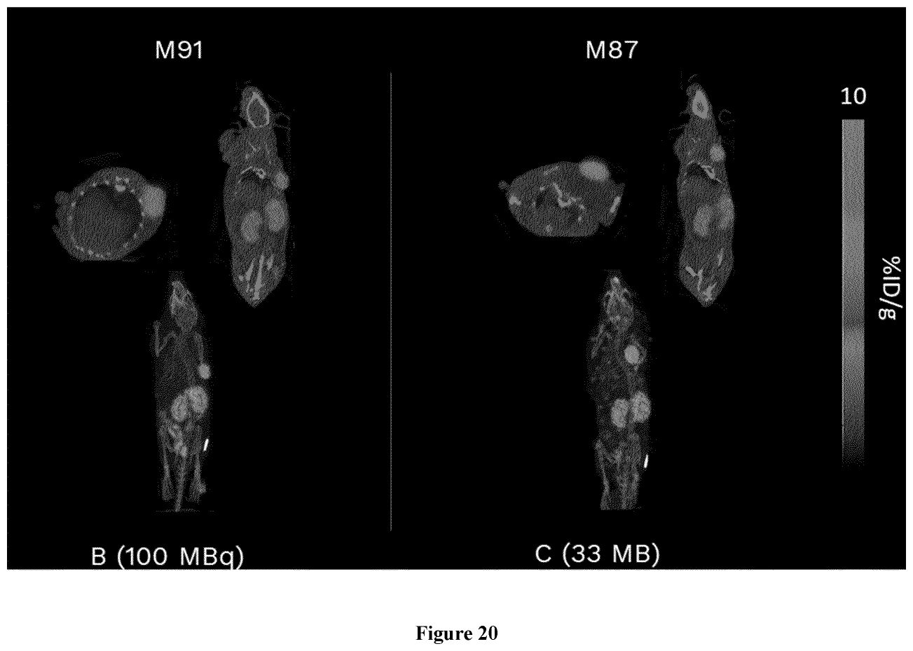

One week later the 6 satellite mice were then assessed again for radioactivity uptake by whole-body SPECT/CT imaging at 4 h after the single ¹⁷⁷Lu-DPI-4452 administration using parameters as described above. The results are shown in FIGS. 15 to 20.

For both models blood sampling for hematology was performed on all animals in group A-D at study day −1, 7, 14 and at study end. Each mouse was restrained and 200 μl blood was obtained from the sublingual or jugular vein in EDTA-tubes and analyzed on the sampling day on a ProCyte Dx Hematology Analyzer with mouse settings. In addition, for the SK-RC-52 model at study day 14 and at study end (day 43), excess blood from the hematology analysis was spun and plasma was obtained (centrifugation at 2000 g for 10 mins at 4° C. in EDTA tubes). The plasma was analyzed for creatinine and urea concentrations using a KONELAB PRIME 30i instrument (Thermo Fisher Scientific).

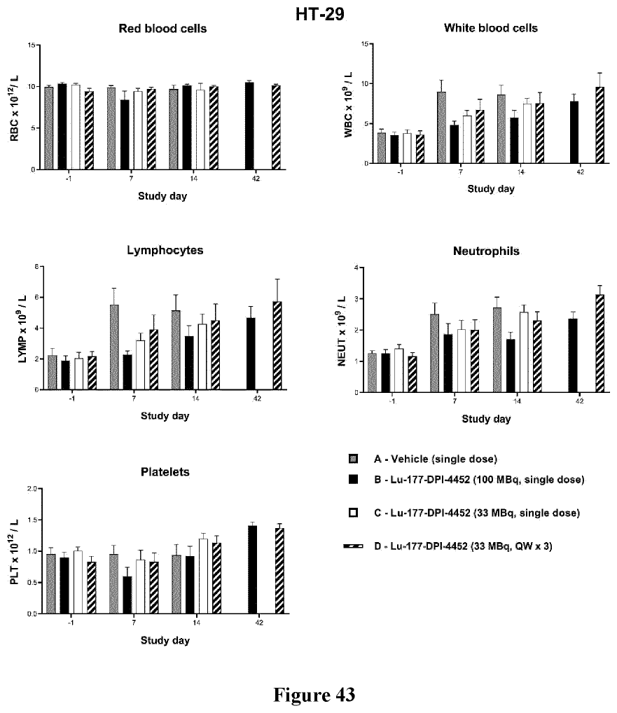

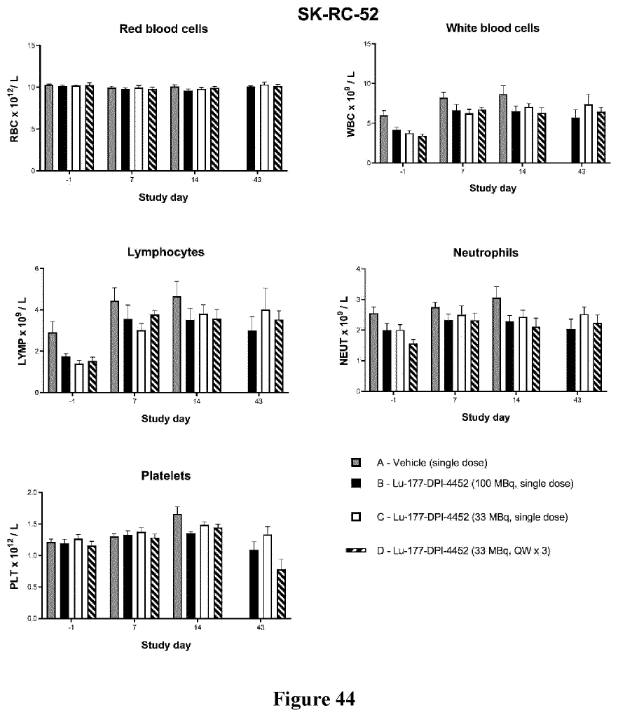

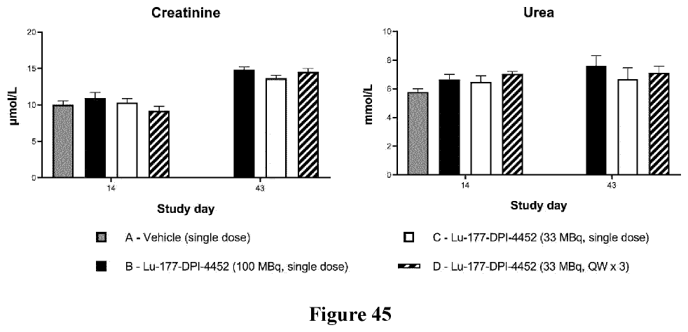

All treatments with ¹⁷⁷Lu-DPI-4452 or ⁶⁸Ga-DPI-4452 were well tolerated in both models. White blood cells, lymphocytes and neutrophils showed increased levels after treatment initiation in all groups for both tumor models when compared to the baseline. Animals dosed with ¹⁷⁷Lu-DPI-4452 (33/100 MBq) showed a relative suppression when compared to animals dosed with vehicle for both tumor models. No differences in creatinine and urea levels between treatment groups were observed at any timepoint. Creatinine values were slightly elevated at study end for the treatment groups, but no control animals were alive at this point for comparison. Results are shown in FIGS. 43 to 45.

SPECT/CT imaging of HT-29 and SK-RC-52 xenografted animals at 4 h after injection of ¹⁷⁷Lu-DPI-4452 demonstrated rapid and high tumor uptake in both models. Tumor uptake remained stable over multiple ¹⁷⁷Lu-DPI-4452 weekly injections in the groups that received three weekly doses of 33 MBq (group D; QW×3). Preferential uptake in the tumor compared to kidney and liver was observed for all groups in both models. A strong and dose-dependent tumor growth inhibition (maximal T/C<20%) was observed in the 100 MBq and the 3×33 MBq treatment groups for the HT-29 model and the 100 MBq, 3×33 MBq, and the 33 MBq treatment groups for the SK-RC-52 model. In both models the dose fractionation (3×33 MBq) seemed beneficial in the long run, and in the SK-RC-52 model led to tumor stasis sustained until study end (day 42). ⁶⁸Ga-DPI-4452 tumor, kidney and liver uptake evaluated by PET imaging closely matched the ¹⁷⁷Lu-DPI-4452 tumor uptake evaluated by SPECT imaging obtained 1 week later for the same animals. Preferential tumor uptake was observed in both models.

### Example 25: Biodistribution Study in Tumor-Bearing Mice with or without Kidney Protection

The biodistribution of [¹¹¹In]In-DPI-4452 and [¹¹¹In]In-DPI-4501 was evaluated in the SK-RC-52 and HT-29 tumor models established subcutaneously in NMRI nude mice, as well as in kidneys, liver and heart (estimate of blood). The effect on tumor uptake of prior injection of gelofusine for kidney protection was investigated in mice bearing the SK-RC-52 tumor. Biodistribution was evaluated using in vivo SPECT/CT imaging. Prior to the in vivo study the compounds (ligands) were initially radiolabeled with In-111 and the stability of radiolabeled compounds was evaluated in buffer at three different timepoints.

The study was performed in female NMRI nude mice (approximately 6 weeks of age) from Janvier Labs, France, housed up to 5 mice per cage.

Indium In-111 chloride (370 MBq/mL at activity reference time) was obtained from Curium and stored at room temperature before use. DPI-4452 and DPI-4501 (also referred to in the present application as “3BP-4452” and “3BP-4501”, respectively) were stored at −20° C. before use.

DPI-4452 (0.42 mg/mL (199 nmol/mL) solution in 0.1 M HEPES, pH 7) and DPI-4501 (0.40 mg/mL (203 nmol/mL) solution in 0.1 M HEPES, pH 7) were labeled at a molar activity of 30 MBq/nmol peptide according to the following procedure:
In-111 (in 0.02 M HCl) and ligand (i.e., either DPI-4452 or DPI-4501) were mixed, and 1 M sodium acetate, pH 5.5 was added (10% of the final volume).
The reaction mixture was stirred for 25 min at 80° C. at 600 RPM, and then cooled to room temperature and quenched with 5 μL of 50 μM DTP.
The mixture was then diluted with water to a final volume of 10 mL and transferred onto the purification column (previously conditioned with 5 mL 99.9% EtOH followed by 5 mL water). The column was washed with 2 mL water, and then the final product was eluted from the column in EtOH solution (50%)
The sample was formulated in PBS to the final concentration of 150 MBq/mL and 5 nmol/mL.

Quality control was conducted using one HPLC method and one TLC method. The HPLC QC method employed a Thermo Scientific Vanquish HPLC system including a UV detector set at 220 nm, a GABI Nova radiodetector and an XBridge C18 3.5 μm 4.6×50 mm column. Chromatography was conducted at room temperature at a flow rate of 1.5 mL/min using a mobile phase consisting of A: 0.1% trifluoroacetic acid in water; and B: 0.1% trifluoroacetic acid in acetonitrile, according to the following linear gradient: 0-7 min from 5% B to 95% B; 7-8.5 min from 95% B to 5% B, 8.5-11 min: 5% B. The TLC QC method employed 11-cm long plates with an iTLC-SG stationary phase; the mobile phase was 0.1 M citric acid, pH 5.4; the sample volume was 2 μL; the detector was a miniGita OFA Probe. The release criteria for the labeled compounds at EOS (end-of-synthesis) were ≥90% radiochemical purity from both HPLC and TLC methods.

SK-RC-52 is a human renal cell carcinoma cell line derived from one metastatic site in mediastinum of a 61-year-old female patient. HT-29 is a human colorectal adenocarcinoma cell line derived from the primary tumor of a 44-year-old female patient.

SK-RC-52 cells were cultured in RPMI 1640 with GlutaMax-I (Thermo Fisher Scientific #61870044) supplemented with 10% fetal bovine serum+1% penicillin/streptomycin, harvested, washed twice in RPMI 1640 and resuspended at 2×10⁷ cells/mL in RPMI 1640. HT-29 cells were cultured in McCoy's 5a Medium Modified (Sigma #M9309). For inoculation of HT-29 cells, cells were harvested, washed twice in PBS and resuspended at 5×10⁷ cells/mL in PBS. Cells were kept on ice until inoculation.

Animals were anaesthetized (isoflurane, 2-4% in ambient air supplemented with 100% 02) prior to tumor inoculation. The tumor cells (100 μL suspension of either SK-RC-52 cells (2×10⁶ cells/animal) or HT-29 cells (5×10⁶ cells/animal)) were subcutaneously inoculated in the right flank using a 1-mL syringe equipped with a 27 G needle. Tumor growth and animal weight were measured twice per week. Tumor size was measured by caliper and the volume was estimated using the following formula: 0.52×(length×width²). When tumor sizes reached 150-250 mm³, animals were randomized into groups (n=3) of similar average tumor volume and body weight.

Animals were injected intravenously with In-111-labeled compounds (single bolus, 22.3-31.2 MBq, ˜1 nmol ligand, injection volume: 100 μL) in the lateral tail vein using a 29 G syringe. Two additional groups of SK-RC-52 tumor-implanted mice were pretreated with intravenous injection of 100 μL 4% gelofusine, immediately before injection of [¹¹¹In]In-DPI-4452 and [¹¹¹In]In-DPI-4501, respectively.

Whole body SPECT/CT scanning (nanoScan SPECT/CT, Mediso) was performed under anesthesia (isoflurane, ²-4% in ambient air supplemented with 100% 02) at 1 h, 4 h, 24 h and 48 h after injection of [¹¹¹In]In-DPI-4452 or [¹¹¹In]In-DPI-4501 for the SK-RC-52 tumor experiments; in the experiment with the HT-29 tumor, the timepoints for SPECT-CT scan were: 1 h, 4 h, 24 h and 48 h after injection of [¹¹¹In]In-DPI-4452 or [¹¹¹In]In-DPI-4501. CT was performed with helical scan, 300 ms exposure, reconstruction resolution of 250 μm. SPECT was performed with multi-pinhole scan and a 30 s frame time. For the quantification of the radioactivity uptake, regions of interests (ROIs) were drawn over the tumor, kidney, liver, and heart (estimate of blood) identified on the images. Uptake was expressed as percent of injected dose per gram tissue (% ID/g).

### Results:

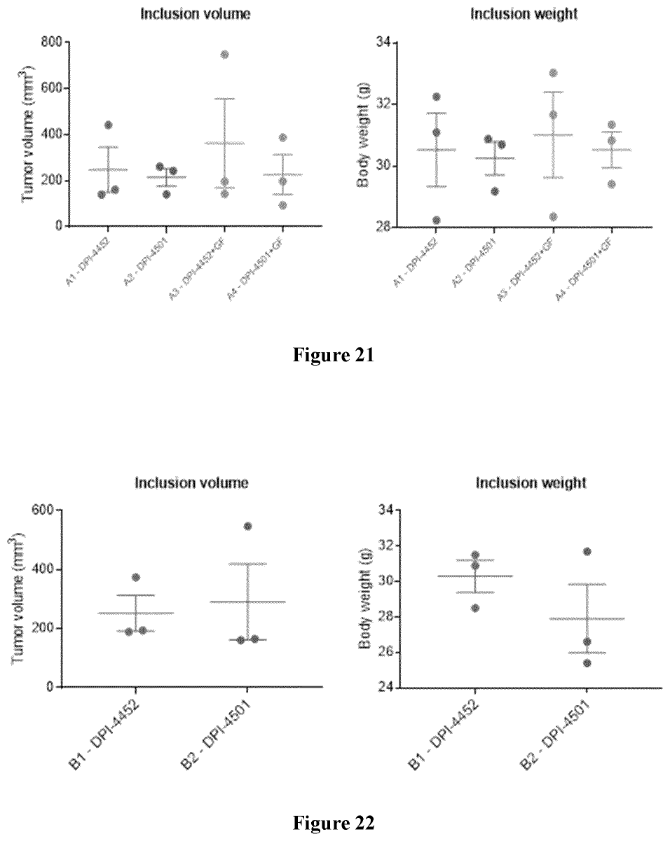

Animals with SK-RC-52 tumor (Group A1-A4) were randomized in four groups with three animals per group at the day of dosing. No significant difference in tumor volume (p=0.80, one-way ANOVA) or animal body weight (P=0.96, one-way ANOVA) was observed between the groups. Tumor volume and body weight at randomization is presented in FIG. 21.

Animals with HT-29 (B1-B2) were randomized in two groups with three animals per group at day −1 (the day before dosing). No significant difference in tumor volume (p=0.80, unpaired t-test) or animal body weight (P=0.32, unpaired t-test) was observed between the groups. Tumor volume and body weight at randomization is presented in FIG. 22.

Both compounds were labeled with an In-111 incorporation of ≥90% on both radio-HPLC and radio-TLC for all labeling preparations. The radiochemical purity (RCP) was ≥95% on radio-HPLC and 100% on radio-TLC. After dosing of all the animals, RCP was found to be ≥95% for [¹¹¹In]In-DPI-4452 and >90% for [¹¹¹In]In-DPI-4501.

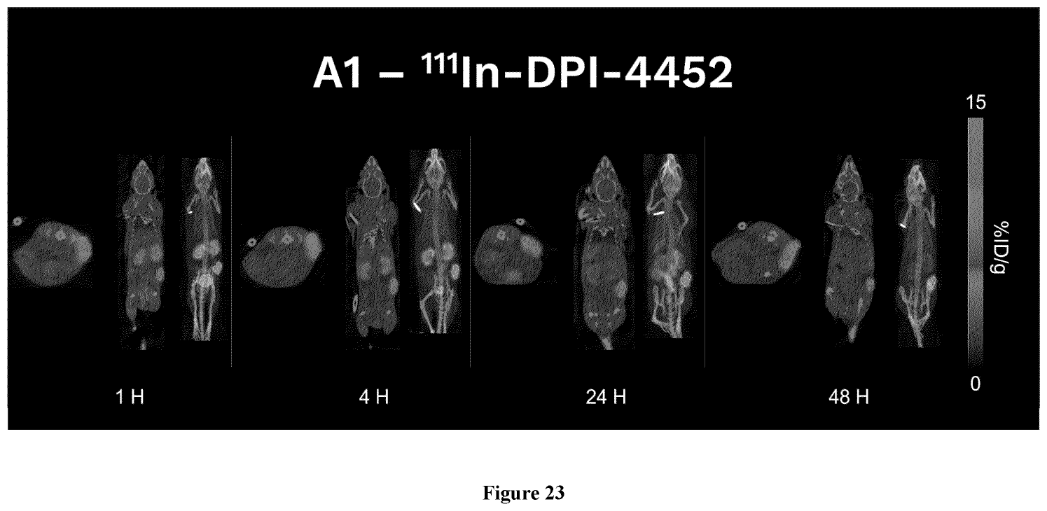

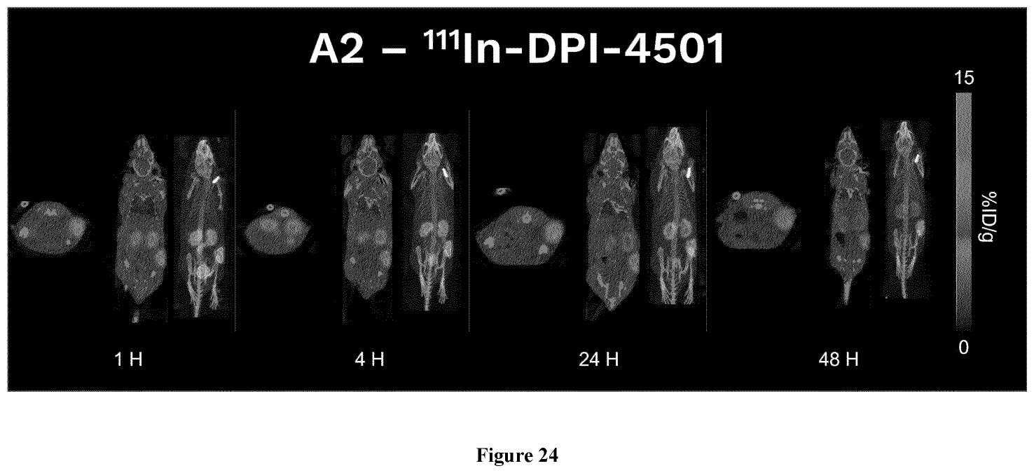

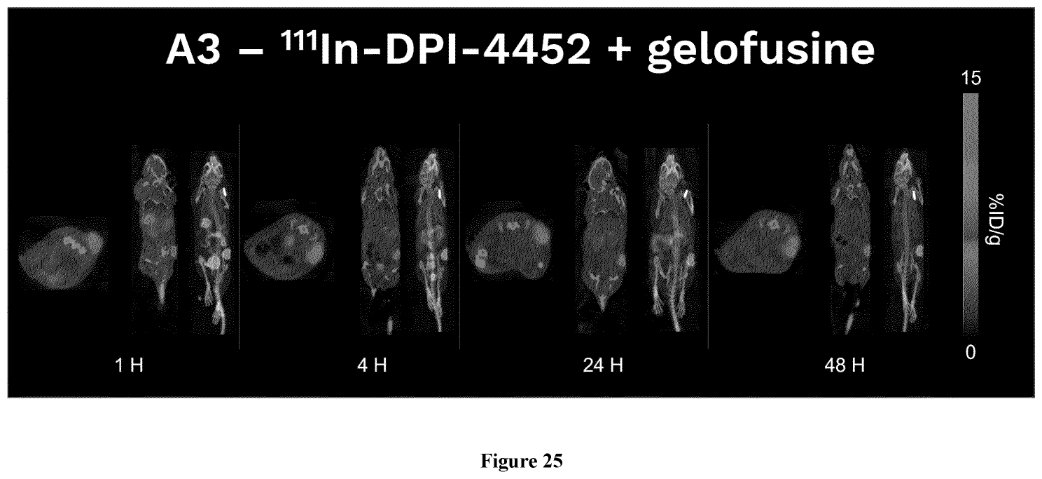

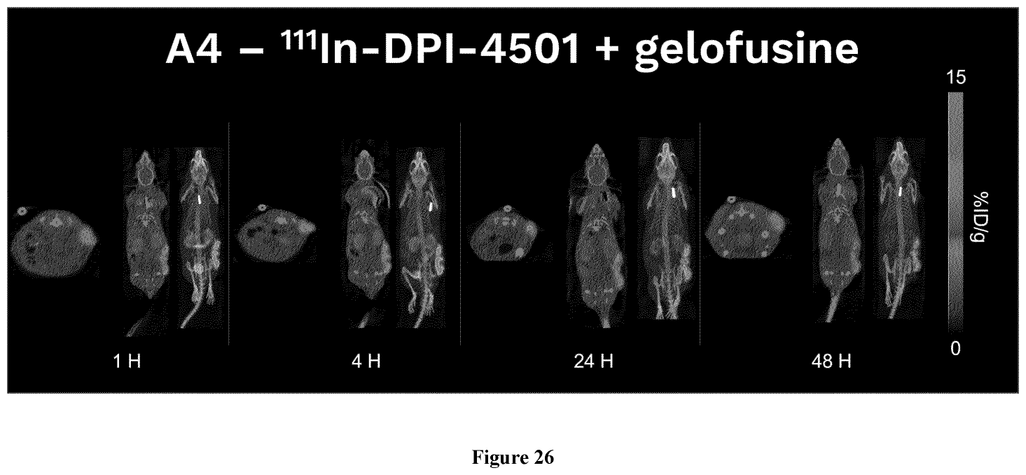

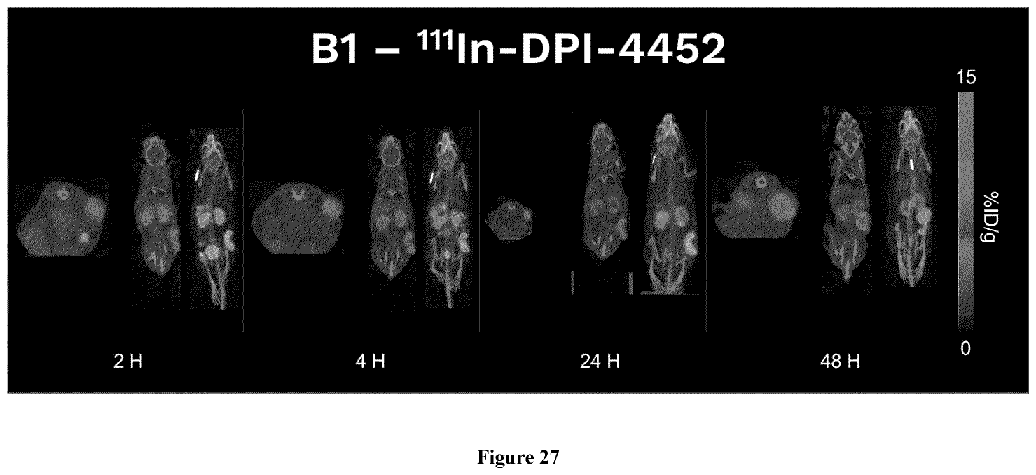

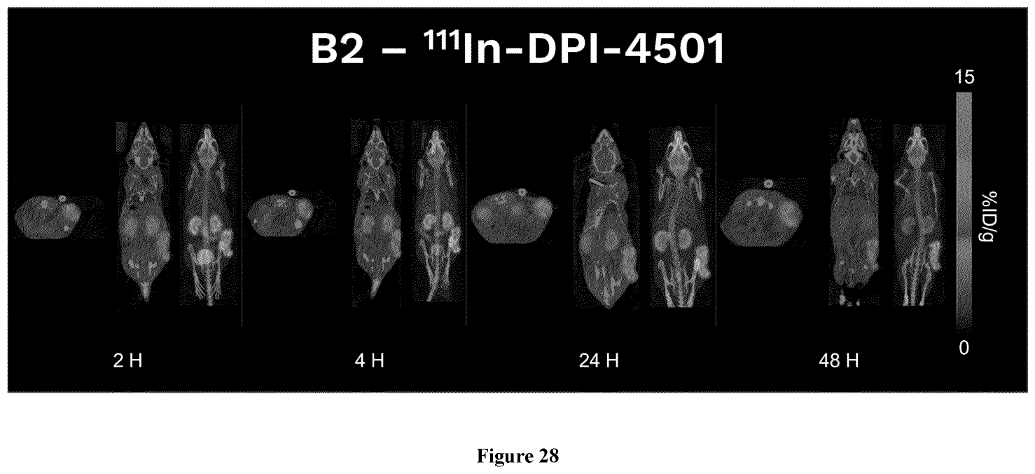

SPECT/CT scans were collected at the above-mentioned time points. Representative axial and coronal images as well as maximum intensity projection (MIP) images of one mouse from each group are shown in FIG. 23-FIG. 28.

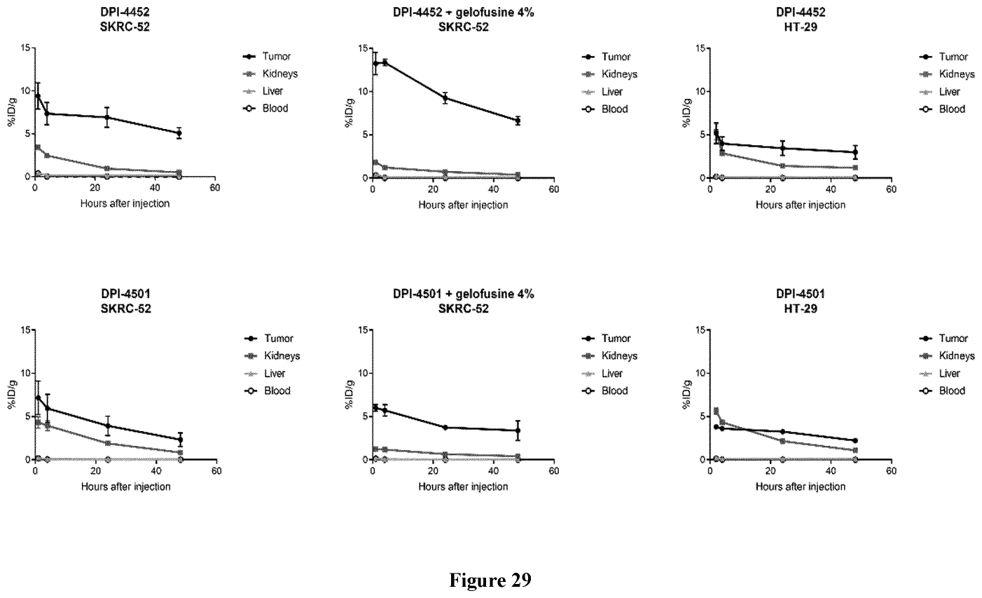

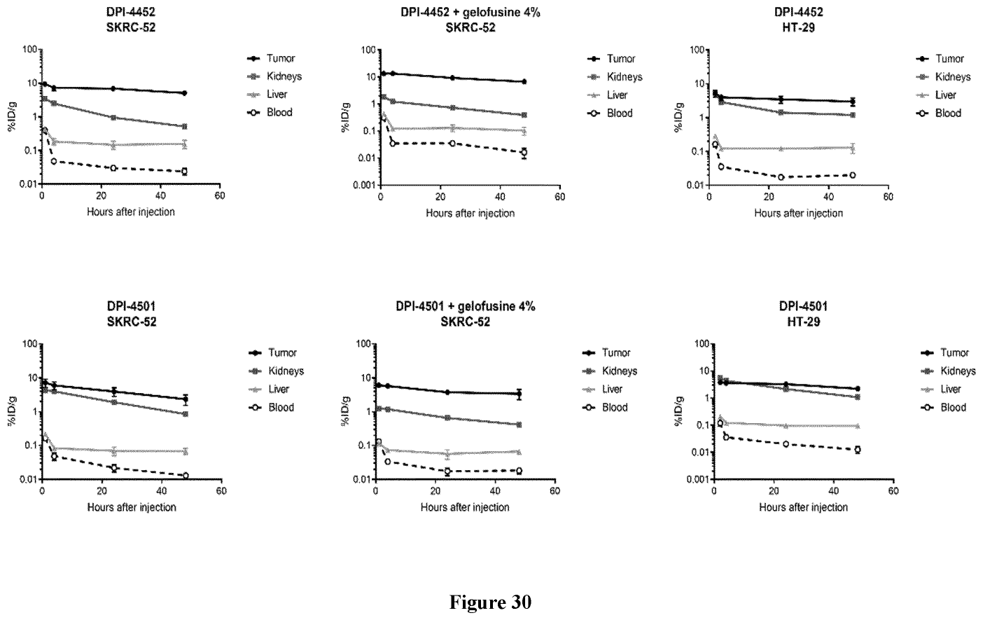

Distribution in tumor, kidneys, liver and blood measured as % ID/g are presented in FIGS. 29 and 30 on a linear and logarithmic scale, respectively. Uptake time profiles of [¹¹¹In]In-DPI-4452 and [¹¹¹In]In-DPI-5401 in SK-RC-52 and HT-29 tumors are presented in Table 25.

TABLE 25
Uptake time profiles of In-111-labeled DPI-4452 and DPI-4501 in SK-RC-52 and HT-29 tumors. Uptake in SK-RC-52 tumor
was compared with injection of 4% gelofusine immediately before injection of compounds. N = 3/group, Mean ± SEM.

| | SK-RC-52 | | | | | |
|---|---|---|---|---|---|---|
| | | A3 | | A4 | HT-29 | |
| | A1 | DPI-4452 + | A2 | DPI-4501 + | B1 | B2 |
| | DPI-4452 | gelofusine | DPI-4501 | gelofusine | DPI-4452 | DPI-4501 |
| Peak Tumor Uptake (% ID/g) | 9.42 ± 1.53 | 13.36 ± 0.39 | 7.17 ± 1.92 | 6.01 ± 0.39 | 5.21 ± 1.22 | 3.81 ± 0.26 |
| % remaining tumor uptake at 24 h p.i. | 73.3 ± 0.8 | 69.6 ± 6.6 | 54.3 ± 1.6 | 62.5 ± 1.9 | 65.8 ± 3.8 | 80 ± 4.4 |
| Tumor/kidney uptake ratio at 24 h p.i. | 7.3 ± 1.3 | 12.7 ± 1.1 | 2.0 ± 0.1 | 5.8 ± 0.3 | 2.6 ± 0.8 | 1.5 ± 0.1 |
| Tumor/Liver uptake ratio at 4 h p.i. | 43.4 ± 16.2 | 111.2 ± 9.3 | 70.2 ± 17.0 | 81.4 ± 14.0 | 32.2 ± 6.1 | 29.8 ± 2.5 |
| Tumor/Blood uptake ratio at 4 h p.i. | 153.3 ± 7.5 | 428.3 ± 115.6 | 127.9 ± 20.3 | 178.6 ± 39.3 | 111.8 ± 18.3 | 104.5 ± 10.2 |
| Peak Kidney uptake (% ID/g) | 3.43 ± 0.24 | 1.84 ± 0.14 | 4.33 ± 1.04 | 1.26 ± 0.12 | 5.37 ± 1.45 | 5.61 ± 0.57 |
| Kidney uptake at 24 h p.i. (% ID/g) | 0.95 ± 0.05 | 0.73 ± 0.2 | 1.9 ± 0.4 | 0.65 ± 0.05 | 1.41 ± 0.11 | 2.16 ± 0.36 |
| Liver uptake at 4 h p.i. (% ID/g) | 0.18 ± 0.04 | 0.12 ± 0.01 | 0.08 ± 0.01 | 0.07 ± 0.01 | 0.12 ± 0.005 | 0.12 ± 0.01 |
| Blood uptake at 4 h p.i. (% ID/g) | 0.05 ± 0.01 | 0.03 ± 0.01 | 0.05 ± 0.01 | 0.03 ± 0.003 | 0.04 ± 0.002 | 0.04 ± 0.005 |
| Time to peak uptake in tumor (h) | 1 | 4 | 1 | 1 | 2 | 2 |

For the SK-RC-52 tumor model, peak tumor uptake was observed at 1 hour post injection for [¹¹¹In]In-DPI-4452 (9.42±1.53% ID/g), [¹¹¹In]In-DPI-4501 (7.17±1.92% ID/g) and [¹¹¹In]In-DPI-4501+gelofusine (6.01±0.39% ID/g). For [¹¹¹In]In-DPI-4452+gelofusine peak tumor uptake was observed at 4 hours post injection (13.36±0.39% ID/g) (Table 25). Peak tumor uptake was higher for [¹¹¹In]In-DPI-4452 (9.42±1.53% ID/g) compared to [¹¹¹In]In-DPI-4501 (7.17±1.92% ID/g); however, the difference was not statistically significant (unpaired t-test, p=0.41). For the HT-29 tumor model, peak uptake was observed at 2 hours post injection for both [¹¹¹In]In-DPI-4452 and [¹¹¹In]In-DPI-4501. The peak tumor uptake in HT-29 was higher for [¹¹¹In]In-DPI-4452 (5.21±1.22% ID/g) than [¹¹¹In]In-DPI-4501 (3.81±0.26% ID/g); however, the difference was not statistically significant (unpaired t-test, p=0.33).

Injection of gelofusine immediately before injection of compounds in the SK-RC-52 tumor model resulted in an increased peak tumor uptake for [¹¹¹In]In-DPI-4452 (13.36±0.39% ID/g with gelofusine compared to 9.42±1.53% ID/g without gelofusine, p=0.07). Injection of gelofusine immediately before injection of [¹¹¹In]In-DPI-4501 had no significant impact on the peak tumor uptake (6.01±0.39% ID/g with gelofusine compared with 7.17±1.92% ID/g without gelofusine, p=0.59).

For both [¹¹¹In]In-DPI-4452 and [¹¹¹In]In-DPI-4501, injection of gelofusine immediately before injection of compounds significantly reduced peak kidney uptake (at 1-2 hours post injection) ([¹¹¹In]In-DPI-4452: 3.43±0.24% ID/g without gelofusine versus 1.84±0.14% ID/g with gelofusine, p=0.004; [¹¹¹In]In-DPI-4501: 4.33±1.04% ID/g without gelofusine versus 1.26±0.12% ID/g with gelofusine, p=0.04) and kidney uptake at 24 hours post injection ([¹¹¹In]In-DPI-4452: 0.95±0.05% ID/g without gelofusine versus 0.73±0.02% ID/g with gelofusine, p=0.01; [¹¹¹In]In-DPI-4501: 1.90±0.40% ID/g without gelofusine versus 0.65±0.05% ID/g with gelofusine, p=0.04).

Injection of 4% gelofusine immediately before injection of compounds resulted in an increase in tumor/kidney uptake ratio at 24 hours post injection, tumor/liver uptake ratio at 4 hours post injection and tumor/blood uptake ratios at 4 hours post injection for both DPI-4452 and DPI-4501 (Table 25).

### Conclusion:

For both [¹¹¹In]In-DPI-4452 and [¹¹¹In]In-DPI-4501, tumor uptake was higher than kidney uptake; uptake in blood and liver had decreased to background level by 4 h p.i.; peak tumor uptake was typically 7-9% ID/g tissue, yet consistently slightly higher after injection of [¹¹¹In]In-DPI-4452 than [¹¹¹In]In-DPI-4501. The two tumor models SK-RC-52 and HT-29 gave similar results, yet uptake tended to be higher in SK-RC-52 tumor.

Injection of gelofusine immediately before injection of labeled compound in the SK-RC-52 tumor model resulted in: a significant decrease in kidney uptake for both [¹¹¹In]In-DPI-4452 and [¹¹¹In]In-DPI-4501; similar ([¹¹¹In]In-DPI-4501) or increased ([¹¹¹In]In-DPI-4452) tumor uptake; significant increase in tumor/kidney uptake ratio for both compounds, despite preferential uptake in tumor already without gelofusine.

### Example 26: Biodistribution Study in Healthy Dog

The biodistribution of [¹¹¹In]In-DPI-4452 and [¹¹¹In]In-DPI-4501 in male (n=2) and female (n=2) beagle dogs after i.v. (intravenous) administration was evaluated using in vivo SPECT-CT imaging at 1, 4 and 48 hours post injection. Blood pharmacokinetics and urine excretion data were determined from radioactivity concentration data determined by gamma-counting of respective samples.

The test compound mass dose level allometrically corresponded to a human dose of around 250 μg. The radioactivity dose was selected based on experience of the scanner sensitivity for In-111.

Before dosing, the dogs were fasted for a minimum of 6 hours, and a maximum of 24 hours before dosing due to sedation/anesthesia for SPECT/CT scanning and urine sampling at 1 and 4 hours after dosing. Furthermore, the animals were fasted before imaging and urine sampling at the 48-hour timepoint. The animals had ad libitum access to domestic quality drinking water.

Indium In-111 chloride (370 MBq/mL at activity reference time) was obtained from Curium and stored at room temperature before use. DPI-4452 and DPI-4501 (also referred to in the present application as “3BP-4452” and “3BP-4501”, respectively) were stored at −20° C. before use.

DPI-4452 (0.42 mg/mL (199 nmol/mL) solution in 0.1 M HEPES, pH 7) and DPI-4501 (0.40 mg/mL (203 nmol/mL) solution in 0.1 M HEPES, pH 7) were labeled at a molar activity of 15 MBq/nmol ligand (i.e. 115:1 stochiometric ratio of ligand:In-111) according to the following procedure:
In-111 (in 0.02 M HCl) and ligand (i.e., either DPI-4452 or DPI-4501) were mixed, and 1 M sodium acetate, pH 5.5 buffer was added (10% of the final volume).
The reaction mixture was stirred for 25 min at 80° C. at 600 rpm, and then quenched with 5 μL of 50 μM DTPA and cooled to room temperature.
The mixture was then diluted with water to a final volume of 10 mL and transferred onto the purification column (previously conditioned with 5 mL 99.9% EtOH followed by 5 mL water). The column was washed with 2 mL water, and then the final product was eluted from the column in EtOH solution (50%)
The sample was formulated in dPBS to the final concentration of 125 MBq/mL.

The formulations were kept at room temperature from labeling until dosing.

Quality control was conducted using one HPLC method and one TLC method. The HPLC QC method employed a Thermo Scientific Vanquish HPLC system including a UV detector set at 220 nm, a GABI Nova radiodetector, and an XBridge C18 3.5 μm 4.6×50 mm column. Chromatography was conducted at room temperature at a flow rate of 1.5 mL/min using a mobile phase consisting of A: 0.1% trifluoroacetic acid in water; and B: 0.1% trifluoroacetic acid in acetonitrile, according to the following linear gradient: 0-7 min from 5% B to 95% B; 7-8.5 min from 95% B to 5% B, 8.5-11 min: 5% B. The TLC QC method employed 11-cm long plates with an iTLC-SG stationary phase; the mobile phase was 0.1M citric acid, pH 5.4; the sample volume was 2 μL; the detector was a miniGita OFA Probe. The release criteria for the labeled compounds at EOS (end-of-synthesis) were ≥90% radiochemical purity from both the HPLC and the TLC methods.

For blood sampling, the dogs had a venflon inserted (BD 22 G) in v. cephalica (front leg) or in v. saphena (hind leg). For dosing, the dogs had a venflon inserted on an opposite leg of the blood sampling, which was removed after dosing. The dogs received a single intravenous dose of 250 MBq In-111-labeled compound (36 and 38 nmol ligand of DPI-4452 and DPI-4501, respectively); the dose volume was 2 mL. The activity in the syringe was measured and the residual activity in the syringe and venflon was measured after dosing in a dose calibrator. The procedure for taking a sample of urine from live animals was cystocentesis with a 21 G cannula and a 5 mL syringe for females, whereas urine sample from males was taken through a urine catheter (placed 10-15 minutes prior to scheduled urine sampling timepoint). Collected urine was mixed to homogenize concentration.

Whole body SPECT/CT scanning (Clinical D670 SPECT/CT, GE) was performed under anesthesia 1, 4 and 48 hours after injection of the In-111-labeled compounds. The acquisition time enabled a minimum number of counts for good image resolution and quality while not exceeding the maximum time allowed for the animal to be in anesthesia for the 3 field of views needed to include the entire animal. During the CT part of the SPECT/CT scanning an intravenous infusion of an iodine-containing contrast medium (Iohexol, 300 mg/mL) was administrated as a 1 mL/kg volume and at a flow rate of 1 mL/sec to improve the delineation of the organs during image analysis.

The animals were transported in a sedated state to the scanner room on site. Sedation was achieved with 0.1-0.3 mg/kg i.m. (intramuscular)/i.v. (intravenous) Comfortan (methadone 10 mg/mL) and 0.002-0.01 mg/kg i.m./i.v. Dexdomitor® (dexmedetomidine 0.5 mg/mL). Then, anesthesia was induced by 3-6 mg/kg i.v. propofol (10 mg/mL). The dogs were intubated and connected to an anesthetic vaporizer and assigned 100% medicinal oxygen mixed with isoflurane (approx. 1.5-3%). The animals were sedated and anesthetized for 60-120 minutes.

For the quantification of the radioactivity uptake, a region of interest was drawn over eight (8) organs identified in the image data. The organs of interest were kidney, liver incl. gallbladder, gonads, bone marrow, lung with pleura, stomach, small intestine, and colon. Uptake was expressed as % ID/g (percent of injected dose per gram tissue) and SUV (standardized uptake value). Standardized uptake value (SUV) is widely used in clinical practice, calculated as the ratio of tissue radioactivity concentration (e.g. in kBq/ml) at a given time, and the administered dose per body weight (e.g. in MBq/kg).

Blood sampling was done at the following timepoints after dosing: 5 min, 10 min, 20 min, 30 min, 45 min, 1 h, 2 h, 4 h, 8 h, 24 h, 48 h and 72 h. Blood sampling was performed through the implanted venflon up to 4 h post injection, and then through v. jugularis with a cannula (BD 21 G) and syringe (2 mL), or through v. cephalica with a cannula (21 G) if necessary due to temperament of dog, or anatomical reasons. Activity in blood samples was measured in a calibrated gamma counter (Hidex Automatics Gamma Counter) for 60 seconds using an energy window of 15-2047 keV.

Urine sampling was performed at 1, 4 and 48 hours post injection. The urine sampling in female dogs was performed using cystocenteses with a cannula (21 G) and syringe while the dog was under sedation or anesthesia. In male dogs, urine was sampled through a urine catheter which was placed during sedation. From each urine sample approx. 50 μL-0.5 mL of urine was transferred to 5 mL scintillation vials for activity measurement in the gamma counter for 60 seconds using an energy window of 15-2047 keV.

Blood half-life values were calculated by fitting bi-exponential equation to the measured blood activity concentrations.

### Results:

DPI-4452 was labeled with an incorporation of ≥91% estimated from both radio-HPLC and radio-TLC. The radiochemical purity (RCP) at the end of synthesis (EOS) was ≥97% from radio-HPLC and 100% from radio-TLC. DPI-4501 was labeled with an incorporation of ≥92% estimated from both radio-HPLC and radio-TLC for the dosing of both female and male dogs, while RCP at EOS was ≥95% from radio-HPLC and 100% from radio-TLC. After dosing of all the animals, RCP was found to be ≥93% for [¹¹¹In]In-DPI-4452 and ≥91% for [¹¹¹In]In-DPI-4501.

The actual injected dose was 250 MBq (230 MBq in females dogs dosed with [¹¹¹In]In-DPI-4452) with 36 nmol total ligand. In the injected ¹¹¹In-labelled compound formulation, the ratio of In-complex to total ligand was 0.38% (0.41% in females dosed with [¹¹¹In]In-DPI-4452).

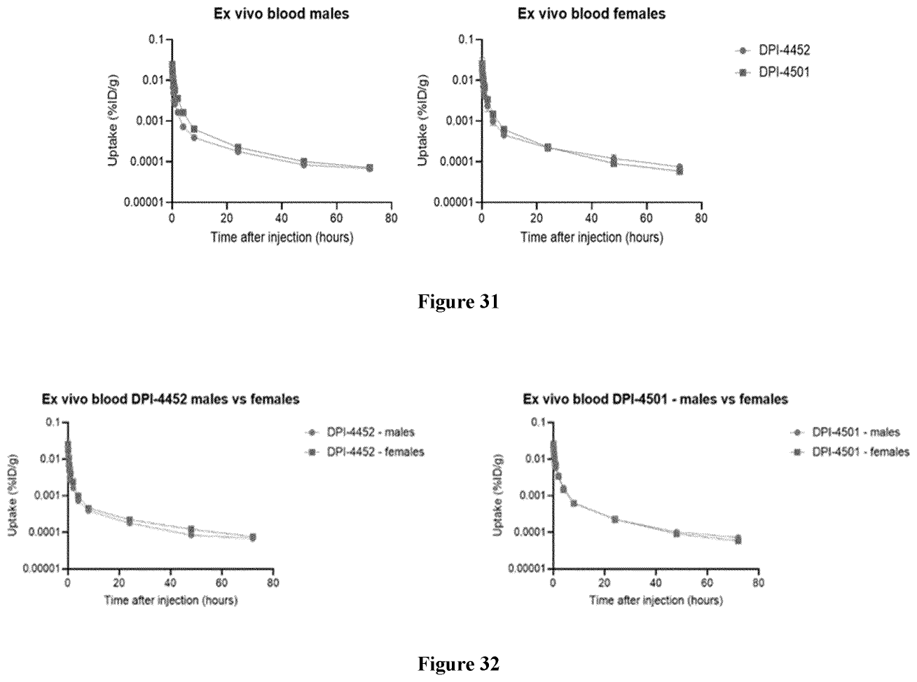

The time course of total radioactivity concentration in dog blood (expressed as % ID/g (% injected dose/g blood), corrected for radioactive decay) after a single IV dose of [¹¹¹In]In-DPI-4452 or [¹¹¹In]In-DPI-4501 is presented in FIG. 31 and FIG. 32, respectively.

There was no difference in blood pharmacokinetic profiles of males and females, after injection of either [¹¹¹In]In-DPI-4452 or [¹¹¹In]In-DPI-4501. The average blood kinetic profile of radioactivity after injection of [¹¹¹In]In-DPI-4452 followed an apparent biphasic course with one early distribution phase (timepoints from 5 minutes to 1 hour) characterized by a half-life (T_{1/2}) of 5.43 min and one late elimination phase (timepoints from 24 hours to 72 hours) with T_{1/2}=55.6 min. Similarly, the kinetic profile of radioactivity after injection of [¹¹¹In]In-DPI-4501 followed an apparent biphasic course with one early distribution phase (timepoints from 5 minutes to 1 hour) with T_{1/2}=5.88 min and one late elimination phase (timepoints from 24 hours to 72 hours) with T_{1/2}=68.9 min.

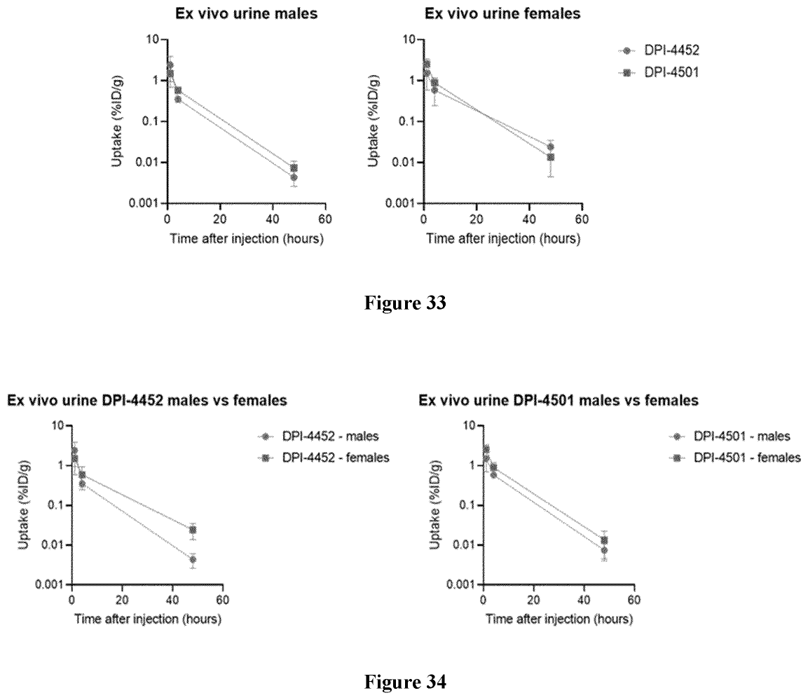

Radioactivity concentration in urine was determined at 1 h, 4 h and 4 8 h post injection (FIG. 33 and FIG. 34). Similar kinetic profiles were obtained for the two test compounds, i.e., concentration decreasing with time from about 2% ID/g down to low values of about 0.012% ID/g. No conclusion can be drawn from the apparent gender-related difference observed after injection of [¹¹¹In]In-DPI-4452 due to inter-individual differences in bladder emptying which could not be controlled in this experimental design.

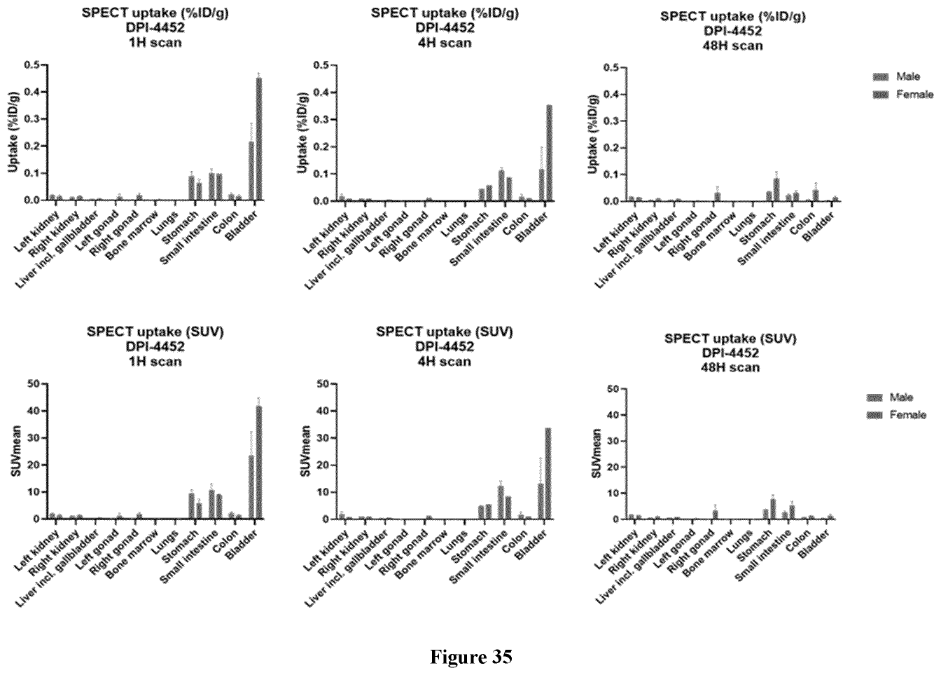

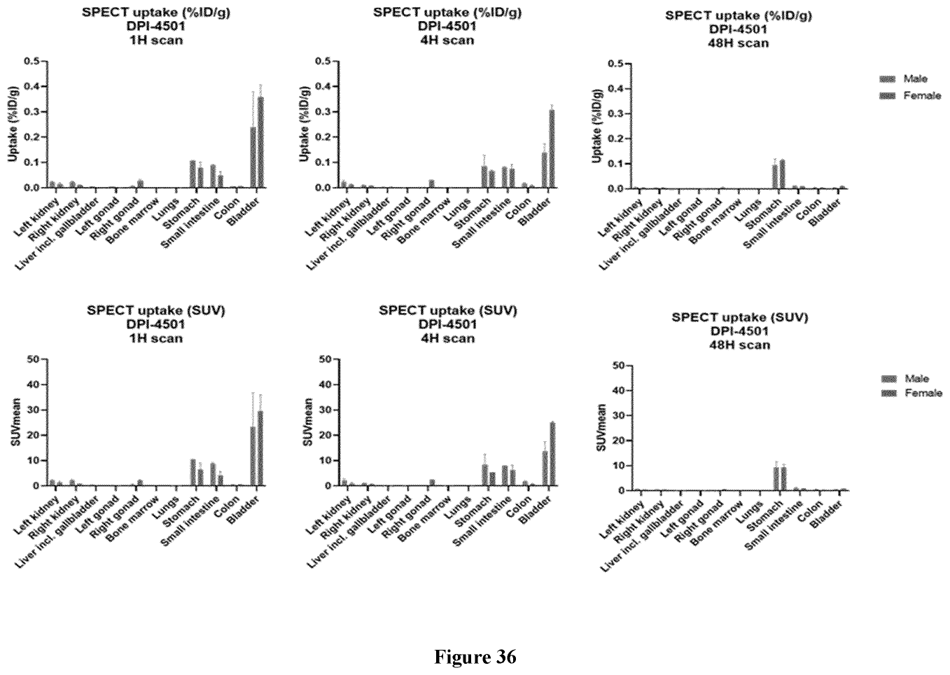

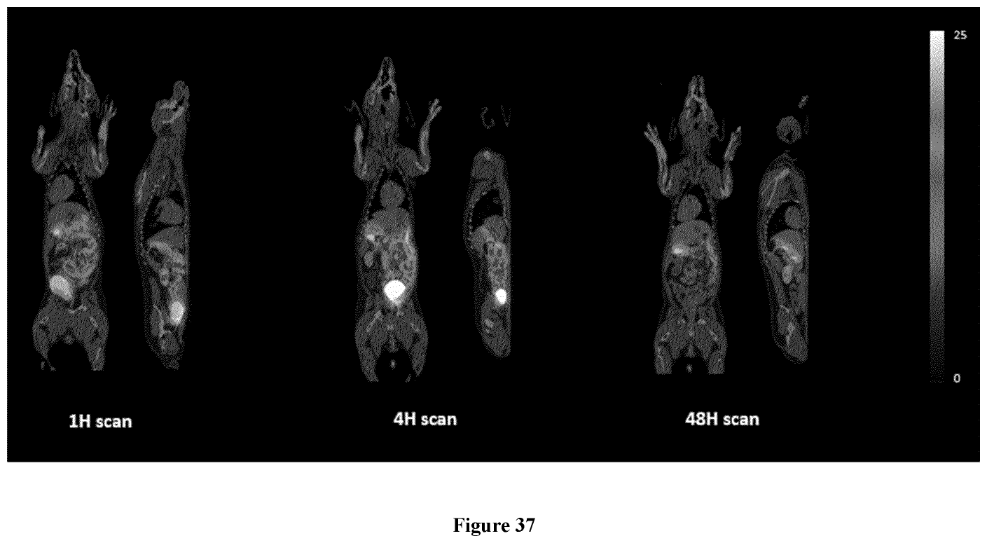

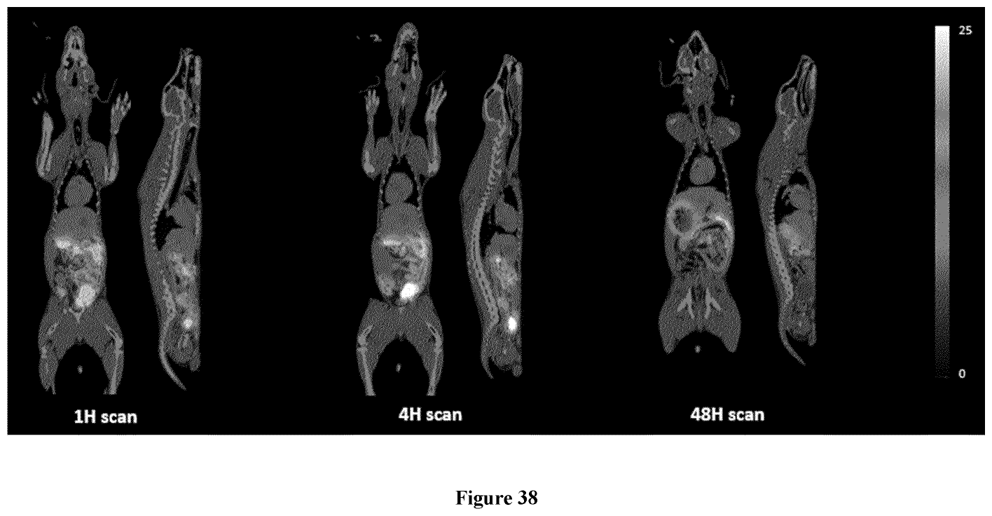

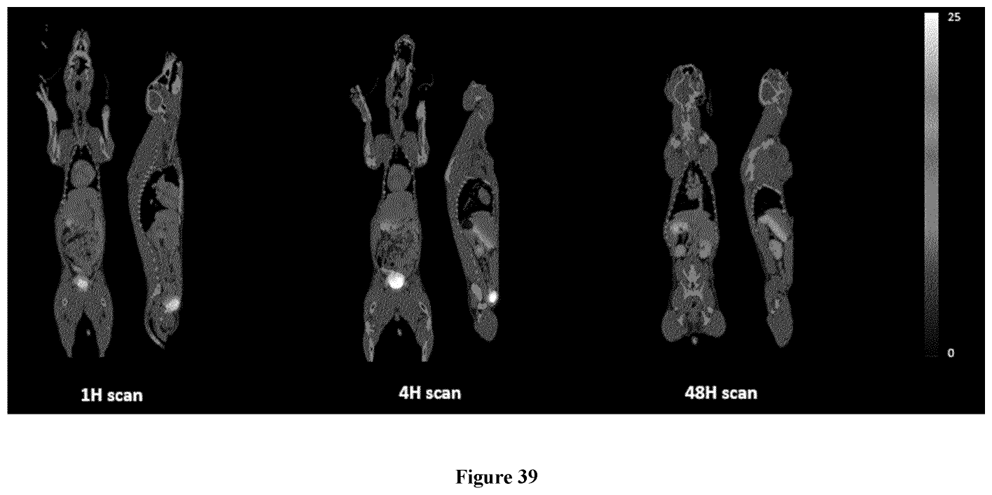

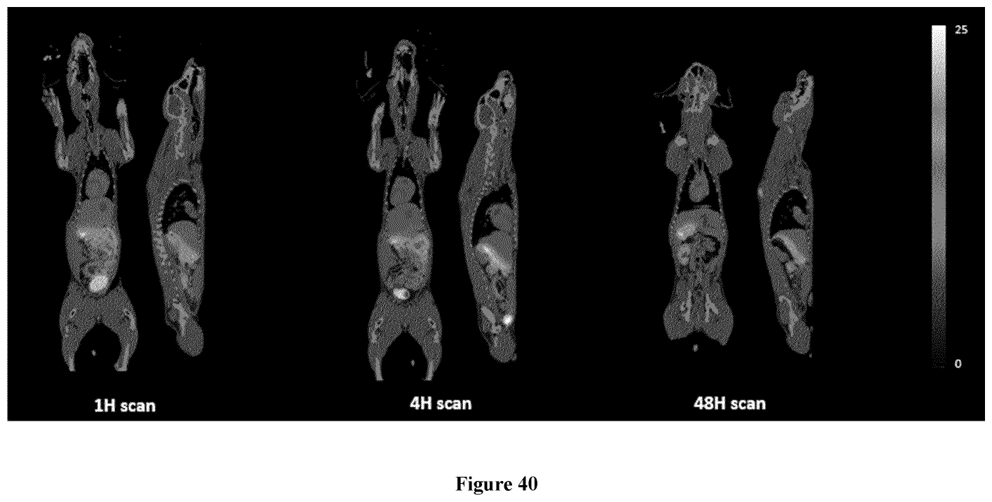

SPECT/CT scans were obtained at 1 h, 4 h and 48 h post-injection. Representative whole-body images are shown in FIG. 37 ([¹¹¹In]In-DPI-4452 in female dogs), FIG. 38 ([¹¹¹In]In-DPI-4452 in male dogs), FIG. 39 ([¹¹¹In]In-DPI-4501 in female dogs) and FIG. 40 ([¹¹¹In]In-DPI-4501 in male dogs). Organ uptake values are shown in FIG. 35 ([¹¹¹In]In-DPI-4452) and FIG. 36 ([¹¹¹In]In-DPI-4501).

Among the selected regions of interest, significant radioactivity uptake was observed in the bladder (0.2-0.5% ID/g or SUV 25-45 at 1 h p.i.), small intestine (ca. 0.1% ID/g or SUV ca. 10 at 1 h p.i.) and the stomach (ca. 0.1% ID/g or SUV ca. 10 at 1 h p.i.), whereas uptake in the other organs was very low, typically below 0.2% ID/g or SUV below 2, which may be considered as background level (gonads, kidneys, liver with gallbladder, colon, bone marrow, lungs).

Radioactivity uptake in the organs tended to decrease with time, from 1 h to 4 h and to 48 h post-injection in the bladder and in the small intestine, whereas no trend was seen in the organs with background (very low) uptake levels, and sustained uptake was observed in the stomach.

The high level of radioactivity in the bladder is likely resulting from extensive excretion of radioactivity in urine. In females, average bladder uptake tended to be approximatively twice as high as in males at 1-4 h p.i., yet variability was high, in line with inter-individual variability in the presence of not-yet excreted urine.

The high levels of radioactivity in the small intestine and stomach likely result from the presence of naturally expressed target CAIX in those organs in the dog, since both test compounds have been demonstrated to bind dog CAIX with similar potency to human CAIX (see Example 28), and expression of CAIX in those organs in the dog has been documented [The Human Protein Atlas, https://www.proteinatlas.org/ENSG00000107159-CA9/tissue]. Levels observed in the colon were low and not significantly different from background.

As apparent in FIG. 35 and FIG. 36, the right gonad of the females tended to have markedly higher uptake than the left gonad, which has been interpreted as being due to spillover from the GI tract and stomach rather than specific uptake in the gonads. The differences here should therefore be anticipated to have origin in the placement of the right gonad rather than specific target binding.

### Example 27: Dosimetry Study Based on Dog Biodistribution Data

Dosimetry of ¹¹¹In radiation based on organ uptake data from the above dog biodistribution study was conducted as follows. The area under the time activity curves was calculated using linear interpolation between datapoints, assuming residual activity at the last timepoint to decay fully in the tissue. The number of disintegrations per gram tissue per administered MBq was calculated and extrapolated to human using the % kg/g method (Kirschner et al., J. Nucl. Med. 1975, 16(3), 248-249) using individual animal body weights, ICRP89 human phantom body weights and organ masses to calculate the number of disintegrations per human organ (ICRP 89, 2002, Basic Anatomical and Physiological Data for Use in Radiological Protection Reference Values. ICRP Publication 89. Ann. ICRP 32 (3-4)), and according to the following formula:

$$[ {( \frac{\%}{{}^{g} organ} )}_{animal} \times {( {}^{kg} TBweight )}_{animal} ] \times {( \frac{{}^{g} organ}{{}^{kg} TBweight} )}_{human} = {( \frac{\%}{organ} )}_{human}$$

This was converted to residence time by division by 3.6E9 dis/MBq/h (number of decays from a constant activity of 1 MBq during a period of 1 h) and entered into OLINDA/EXM 2.0 to calculate human absorbed doses. Output ICRP 103 ED is the effective dose contribution from the individual organs/compartments. Finally, the effective dose is calculated as the average dose to males and females (MIRD Pamphlet 21; Bolch et al. J. Nucl. Med. 2009, 50(3), 477-484).

Estimation of the dosimetry of the ¹⁷⁷Lu-labeled analogue was performed by re-calculation of the individual data points, based on the difference in half-life between In-111 and Lu-177. Then, the subsequent steps were identical to ¹¹¹In dosimetry. Organ-related maximum tolerated dose values established for external beam radiotherapy (From table 3 in Tolerance of Normal Tissue to Therapeutic Radiation, Dr. Emami B, 2013) were used to calculate the maximum allowed injected radioactivity dose (a conservative approach).

Finally, at the projected maximum allowed ¹⁷⁷Lu injected radioactivity dose, the range of possible radiation doses delivered to a human tumor was estimated based on tumor uptake derived from a xenografted mouse study (described above in this document). Since the relative sizes of the xenograft tumor and the whole mouse body are not matching the situation of a human patient with tumor, two different approaches of extrapolation of xenograft tumor dosimetry were used and yielded a range of limit values. One approach consisted in keeping constant % ID (a typically reported method, see Biodistribution and radiation dosimetry of radioiodinated hypericin as a cancer therapeutic, Cona et al. 2013, and Enhancing Treatment Efficacy of ¹⁷⁷Lu-PSMA-617 with the Conjugation of an Albumin-Binding Motif: Preclinical Dosimetry and Endoradiotherapy Studies, Kuo et al., Molecular Pharmaceutics 2018, 15(11), 5183-5191), however leading to overestimation of absorbed dose in small tumors. The other approach consisted in assuming a constant activity concentration (% ID/g) in the tumor tissue, which leads to an underestimation of dose absorbed in small tumors. Thus, the real value of radiation dose absorbed in the tumor will most likely lie between these two different extreme estimates of tumor absorbed radiation dose. A tumor size of 11 g was considered, as the average tumor weight of 5 randomly chosen actual patients in the clinic from a personal discussion between the study director and a nuclear physicist. The purpose of this preclinical tumor dosimetry was to check whether a maximum allowed injected dose of [¹⁷⁷Lu]Lu-DPI-4452 or [¹⁷⁷Lu]Lu-DPI-4501 radioactivity enables to deliver a radiation dose high enough to cause significant damage to the tumor, i.e. at least 50 Gy.

### Results:

The radiation residence time in the different organs was calculated for input into OLINDA (Table 26 and Table 27, for [¹¹¹In]In-DPI-4452; Table 28 and Table 29, for [¹¹¹In]In-DPI-4501). Radiation doses absorbed in human organs were extrapolated using OLINDA (Table 30 and Table 31, for [¹¹¹In]In-DPI-4452; Table 32 and Table 33, for [¹¹¹In]In-DPI-4501). The resulting effective doses were 1.03×10-1 mSv/MBq and 8.44×10⁻² mSv/MBq, respectively (Table 34 and Table 35).

TABLE 26
Input parameters for the male dose calculation
for compound [¹¹¹In]In-DPI-4452.

| Organ | Mass(g) | Residence time (h) |
|---|---|---|
| Adrenals | 14 | 0.00E+00 |
| Brain | 1450 | 0.00E+00 |
| Esophagus | 40 | 0.00E+00 |
| Eyes | 15 | 0.00E+00 |
| Gallbladder Contents | 58 | 3.85E−02 |
| Left colon | 75 | 9.73E−02 |
| Small Intestine | 350 | 2.30E+00 |
| Stomach Contents | 250 | 1.36E+00 |
| Right colon | 150 | 1.95E−01 |
| Rectum | 75 | 9.73E−02 |
| Heart Contents | 510 | 1.70E−02 |
| Heart Wall | 330 | 0.00E+00 |
| Kidneys | 310 | 5.05E−01 |
| Liver | 1800 | 1.19E+00 |
| Lungs | 1200 | 2.55E−01 |
| Pancreas | 140 | 0.00E+00 |
| Prostate | 17 | 0.00E+00 |
| Salivary Glands | 85 | 0.00E+00 |
| Red Marrow | 1170 | 8.24E−02 |
| Cortical Bone | 4400 | 0.00E+00 |
| Trabecular Bone | 1100 | 0.00E+00 |
| Spleen | 150 | 0.00E+00 |
| Testes | 35 | 2.77E−03 |
| Thymus | 25 | 0.00E+00 |
| Thyroid | 20 | 0.00E+00 |
| Urinary Bladder Contents | 211 | 1.09E+00 |
| Total Body | 73000 | 4.09E+01 |

TABLE 27
Input parameters for the female dose calculation
for compound [¹¹¹In]In-DPI-4452.

| Organ | Mass(g) | Residence time (h) |
|---|---|---|
| Adrenals | 13 | 0.00E+00 |
| Brain | 1300 | 0.00E+00 |
| Breasts | 500 | 0.00E+00 |
| Esophagus | 35 | 0.00E+00 |
| Eyes | 15 | 0.00E+00 |
| Gallbladder Contents | 48 | 5.53E−02 |
| Left colon | 80 | 1.408−01 |
| Small Intestine | 280 | 2.65E+00 |
| Stomach Contents | 230 | 2.68E+00 |
| Right colon | 160 | 2.80E−01 |
| Rectum | 80 | 1.40E−01 |
| Heart Contents | 370 | 1.79E−02 |
| Heart Wall | 250 | 0.00E−00 |
| Kidneys | 275.5 | 4.72E−01 |
| Liver | 1400 | 1.61E+00 |
| Lungs | 950 | 3.65E−01 |
| Ovaries | 11 | 3.26E−03 |
| Pancreas | 120 | 0.00E+00 |
| Salivary Glands | 70 | 0.00E+00 |
| Red Marrow | 900 | 1.07E−01 |
| Cortical Bone | 3200 | 0.00E+00 |
| Trabecular Bone | 800 | 0.00E+00 |
| Spleen | 130 | 0.00E+00 |
| Thymus | 20 | 0.00E+00 |
| Thyroid | 17 | 0.00E+00 |
| Urinary Bladder Contents | 160 | 1.50E+00 |
| Uterus | 80 | 0.00E+00 |
| Total Body | 60000 | 2.72E+01 |

TABLE 28
Input parameters for the male dose calculation
for compound [¹¹¹In]In-DPI-4501.

| | | Kinetics Value |
|---|---|---|
| Source Organ Name | Mass [g] | [MBq-h/MBq] |
| Adrenals | 14 | 0.00E+00 |
| Brain | 1450 | 0.00E+00 |
| Esophagus | 40 | 0.00E+00 |
| Eyes | 15 | 0.00E+00 |
| Gallbladder Contents | 58 | 1.51E−02 |
| Left colon | 75 | 7.61E−02 |
| Small Intestine | 350 | 1.35E+00 |
| Stomach Contents | 250 | 3.02E+00 |
| Right colon | 150 | 1.52E−01 |
| Rectum | 75 | 7.61E−02 |
| Heart Contents | 510 | 2.51E−02 |
| Heart Wall | 330 | 0.00E+00 |
| Kidneys | 310 | 3.29E−01 |
| Liver | 1800 | 4.68E−01 |
| Lungs | 1200 | 4.77E−02 |
| Pancreas | 140 | 0.00E+00 |
| Prostate | 17 | 0.00E+00 |
| Salivary Glands | 85 | 0.00E+00 |
| Red Marrow | 1170 | 2.88E−02 |
| Cortical Bone | 4400 | 0.00E+00 |
| Trabecular Bone | 1100 | 0.00E+00 |
| Spleen | 150 | 0.00E+00 |
| Testes | 35 | 1.45E−03 |
| Thymus | 25 | 0.00E+00 |
| Thyroid | 20 | 0.00E+00 |
| Urinary Bladder Contents | 211 | 1.62E+00 |
| Total Body | 73000 | 1.74E+01 |

TABLE 29
Input parameters for the female dose calculation
for compound [¹¹¹In]In-DPI-4501.

| | | Kinetics Value |
|---|---|---|
| Source Organ Name | Mass [g] | [MBq-h/MBq] |
| Adrenals | 13 | 0.00E+00 |
| Brain | 1300 | 0.00E+00 |
| Breasts | 500 | 0.00E+00 |
| Esophagus | 35 | 0.00E+00 |
| Eyes | 15 | 0.00E+00 |
| Gallbladder Contents | 48 | 9.73E−03 |
| Left colon | 80 | 5.21E−02 |
| Small Intestine | 280 | 8.76E−01 |
| Stomach Contents | 230 | 2.85E+00 |
| Right colon | 160 | 1.04E−01 |
| Rectum | 80 | 5.21E−02 |
| Heart Contents | 370 | 1.86E−02 |
| Heart Wall | 250 | 0.00E+00 |
| Kidneys | 275.5 | 1.98E−01 |
| Liver | 1400 | 2.84E−01 |
| Lungs | 950 | 4.22E−02 |
| Ovaries | 11 | 8.86E−04 |
| Pancreas | 120 | 0.00E+00 |
| Salivary Glands | 70 | 0.00E+00 |
| Red Marrow | 900 | 3.30E−02 |
| Cortical Bone | 3200 | 0.00E+00 |
| Trabecular Bone | 800 | 0.00E+00 |
| Spleen | 130 | 0.00E+00 |
| Thymus | 20 | 0.00E+00 |
| Thyroid | 17 | 0.00E+00 |
| Urinary Bladder Contents | 160 | 2.02E+00 |
| Uterus | 80 | 0.00E+00 |
| Total Body | 60000 | 1.28E+01 |

TABLE 30
Olinda output data for the human male ICRP-89 phantom per MBq administered
[¹¹¹In]In-DPI-4452. Values are given in mGy/MBq for Beta, Gamma
and Total, whereas ICRP-103 ED and Effective dose is given in mSv/MBq.

| Target Organ | Alpha | Beta | Gamma | Total | ICRP-103 ED |
|---|---|---|---|---|---|
| Adrenals | 0.00E+00 | 1.15E−02 | 9.23E−02 | 1.04E−01 | 9.58E−04 |
| Brain | 0.00E+00 | 1.13E−02 | 4.68E−02 | 5.80E−02 | 5.80E−04 |
| Esophagus | 0.00E+00 | 1.13E−02 | 6.52E−02 | 7.65E−02 | 3.06E−03 |
| Eyes | 0.00E+00 | 1.13E−02 | 4.68E−02 | 5.80E−02 | 0.00E+00 |
| Gallbladder Wall | 0.00E+00 | 1.80E−02 | 8.64E−02 | 1.04E−01 | 9.64E−04 |
| Left colon | 0.00E+00 | 2.43E−02 | 1.07E−01 | 1.31E−01 | 6.37E−03 |
| Small Intestine | 0.00E+00 | 7.72E−02 | 1.42E−01 | 2.19E−01 | 2.02E−03 |
| Stomach Wall | 0.00E+00 | 6.60E−02 | 1.49E−01 | 2.15E−01 | 2.58E−02 |
| Right colon | 0.00E+00 | 2.43E−02 | 9.18E−02 | 1.16E−01 | 5.63E−03 |
| Rectum | 0.00E+00 | 2.43E−02 | 9.68E−02 | 1.21E−01 | 2.78E−03 |
| Heart Wall | 0.00E+00 | 1.16E−02 | 6.11E−02 | 7.27E−02 | 6.71E−04 |
| Kidneys | 0.00E+00 | 3.25E−02 | 9.61E−02 | 1.29E−01 | 1.19E−03 |
| Liver | 0.00E+00 | 1.33E−02 | 7.23E−02 | 8.56E−02 | 3.43E−03 |
| Lungs | 0.00E+00 | 4.23E−03 | 5.41E−02 | 5.83E−02 | 7.00E−03 |
| Pancreas | 0.00E+00 | 1.13E−02 | 1.24E−01 | 1.35E−01 | 1.25E−03 |
| Prostate | 0.00E+00 | 1.13E−02 | 8.99E−02 | 1.01E−01 | 4.67E−04 |
| Salivary Glands | 0.00E+00 | 1.13E−02 | 5.86E−02 | 6.99E−02 | 6.99E−04 |
| Red Marrow | 0.00E+00 | 1.00E−02 | 5.70E−02 | 6.70E−02 | 8.04E−03 |
| Osteogenic Cells | 0.00E+00 | 1.55E−02 | 9.55E−02 | 1.11E−01 | 1.11E−03 |
| Spleen | 0.00E+00 | 1.13E−02 | 8.12E−02 | 9.25E−02 | 8.54E−04 |
| Testes | 0.00E+00 | 1.57E−03 | 4.83E−02 | 4.98E−02 | 1.99E−03 |
| Thymus | 0.00E+00 | 1.13E−02 | 5.38E−02 | 6.51E−02 | 6.01E−04 |
| Thyroid | 0.00E+00 | 1.13E−02 | 5.70E−02 | 6.83E−02 | 2.73E−03 |
| Urinary Bladder Wall | 0.00E+00 | 6.32E−02 | 1.51E−01 | 2.14E−01 | 8.56E−03 |
| Total Body | 0.00E+00 | 1.33E−02 | 5.86E−02 | 7.18E−02 | 0.00E+00 |
| Effective Dose | 8.68E−02 | | | | |

TABLE 31
Olinda output data for the human female ICRP-89 phantom per MBq administered
[¹¹¹In]In-DPI-4452. Values are given in mGy/MBq for Beta, Gamma and
Total, whereas ICRP-103 ED and Effective dose is given in mSv/MBq.

| Target Organ | Alpha | Beta | Gamma | Total | ICRP-103 ED |
|---|---|---|---|---|---|
| Adrenals | 0.00E+00 | 9.28E−03 | 1.14E−01 | 1.23E−01 | 1.14E−03 |
| Brain | 0.00E+00 | 9.09E−03 | 3.87E−02 | 4.78E−02 | 4.78E−04 |
| Breasts | 0.00E+00 | 9.09E−03 | 3.56E−02 | 4.47E−02 | 5.36E−08 |
| Esophagus | 0.00E+00 | 9.09E−03 | 5.55E−02 | 6.46E−02 | 2.59E−03 |
| Eyes | 0.00E+00 | 9.09E−03 | 3.88E−02 | 4.79E−02 | 0.00E+00 |
| Gallbladder Wall | 0.00E+00 | 2.07E−02 | 1.09E−01 | 1.30E−01 | 1.20E−03 |
| Left colon | 0.00E+00 | 2.67E−02 | 1.05E−01 | 1.32E−01 | 6.40E−03 |
| Small Intestine | 0.00E+00 | 1.04E−01 | 1.56E−01 | 2.60E−01 | 2.40E−03 |
| Stomach Wall | 0.00E+00 | 1.26E−01 | 2.62E−01 | 3.88E−01 | 4.66E−02 |
| Right colon | 0.00E+00 | 2.67E−02 | 9.74E−02 | 1.24E−01 | 6.02E−03 |
| Rectum | 0.00E+00 | 2.67E−02 | 1.13E−01 | 1.40E−01 | 3.22E−03 |
| Heart Wall | 0.00E+00 | 9.59E−03 | 6.13E−02 | 7.09E−02 | 6.54E−04 |
| Kidneys | 0.00E+00 | 3.41E−02 | 1.04E−01 | 1.38E−01 | 1.28E−03 |
| Liver | 0.00E+00 | 2.31E−02 | 9.60E−02 | 1.19E−01 | 4.76E−03 |
| Lungs | 0.00E+00 | 7.63E−03 | 5.94E−02 | 6.70E−02 | 8.05E−03 |
| Ovaries | 0.00E+00 | 5.85E−03 | 8.98E−02 | 9.56E−02 | 3.83E−03 |
| Pancreas | 0.00E+00 | 9.12E−03 | 1.36E−01 | 1.45E−01 | 1.34E−03 |
| Salivary Glands | 0.00E+00 | 9.09E−03 | 4.13E−02 | 5.04E−02 | 5.04E−04 |
| Red Marrow | 0.00E+00 | 8.74E−03 | 5.36E−02 | 6.23E−02 | 7.48E−03 |
| Osteogenic Cells | 0.00E+00 | 9.57E−03 | 8.35E−02 | 9.30E−02 | 9.30E−04 |
| Spleen | 0.00E+00 | 9.13E−03 | 1.13E−01 | 1.22E−01 | 1.13E−03 |
| Thymus | 0.00E+00 | 9.13E−03 | 5.01E−02 | 5.92E−02 | 5.47E−04 |
| Thyroid | 0.00E+00 | 9.09E−03 | 4.20E−02 | 5.11E−02 | 2.05E−03 |
| Urinary Bladder Wall | 0.00E+00 | 1.03E−01 | 1.55E−01 | 2.58E−01 | 1.03E−02 |
| Uterus | 0.00E+00 | 9.09E−03 | 1.20E−01 | 1.29E−01 | 5.84E−04 |
| Total Body | 0.00E+00 | 1.24E−02 | 5.66E−02 | 6.90E−02 | 0.00E+00 |
| Effective Dose | 1.19E−01 | | | | |

TABLE 32
Olinda output data for the human male ICRP-89 phantom per MBq administered
[¹¹¹In]In-DPI-4501. Values are given in mGy/MBq for Beta, Gamma
and Total, whereas ICRP-103 ED and Effective dose is given in mSv/MBq.

| Target Organ | Alpha | Beta | Gamma | Total | ICRP-103 ED |
|---|---|---|---|---|---|
| Adrenals | 0.00E+00 | 4.94E−03 | 5.94E−02 | 6.44E−02 | 5.94E−04 |
| Brain | 0.00E+00 | 4.80E−03 | 2.00E−02 | 2.48E−02 | 2.45E−04 |
| Esophagus | 0.00E+00 | 4.80E−03 | 4.90E−02 | 5.38E−02 | 2.15E−03 |
| Eyes | 0.00E+00 | 4.80E−03 | 2.00E−02 | 2.48E−02 | 0.00E+00 |
| Gallbladder Wall | 0.00E+00 | 7.43E−03 | 4.44E−02 | 5.18E−02 | 4.78E−04 |
| Left colon | 0.00E+00 | 1.50E−02 | 6.95E−02 | 8.45E−02 | 4.10E−03 |
| Small Intestine | 0.00E+00 | 4.34E−02 | 8.21E−02 | 1.26E−01 | 1.16E−03 |
| Stomach Wall | 0.00E+00 | 1.26E−01 | 2.32E−01 | 3.58E−01 | 4.30E−02 |
| Right colon | 0.00E+00 | 1.50E−02 | 4.90E−02 | 6.40E−02 | 3.10E−03 |
| Rectum | 0.00E+00 | 1.50E−02 | 5.90E−02 | 7.40E−02 | 1.70E−03 |
| Heart Wall | 0.00E+00 | 5.29E−03 | 4.61E−02 | 5.14E−02 | 4.74E−04 |
| Kidneys | 0.00E+00 | 2.12E−02 | 5.73E−02 | 7.85E−02 | 7.24E−04 |
| Liver | 0.00E+00 | 5.21E−03 | 3.92E−02 | 4.44E−02 | 1.78E−03 |
| Lungs | 0.00E+00 | 7.94E−04 | 3.36E−02 | 3.44E−02 | 4.13E−03 |
| Pancreas | 0.00E+00 | 4.80E−03 | 1.21E−01 | 1.25E−01 | 1.16E−03 |
| Prostate | 0.00E+00 | 4.80E−03 | 5.69E−01 | 6.17E−02 | 2.85E−04 |
| Salivary Glands | 0.00E+00 | 4.80E−03 | 2.53E−02 | 3.01E−02 | 3.01E−04 |
| Red Marrow | 0.00E+00 | 4.21E−03 | 3.00E−02 | 3.42E−02 | 4.10E−03 |
| Osteogenic Cells | 0.00E+00 | 6.55E−03 | 4.68E−02 | 5.33E−02 | 5.33E−04 |
| Spleen | 0.00E+00 | 4.81E−03 | 6.11E−02 | 6.59E−02 | 6.08E−04 |
| Testes | 0.00E+00 | 8.19E−04 | 2.44E−02 | 2.52E−02 | 1.01E−03 |
| Thymus | 0.00E+00 | 4.80E−03 | 2.71E−02 | 3.19E−02 | 2.94E−04 |
| Thyroid | 0.00E+00 | 4.80E−03 | 2.58E−02 | 3.06E−02 | 1.22E−03 |
| Urinary Bladder Wall | 0.00E+00 | 8.20E−02 | 1.58E−01 | 2.40E−01 | 9.59E−03 |
| Total Body | 0.00E+00 | 6.78E−03 | 2.86E−02 | 3.53E−02 | 0.00E+00 |
| Effective Dose | 8.27E−02 | | | | |

TABLE 33
Olinda output data for the human female ICRP-89 phantom per MBq administered
[¹¹¹In]In-DPI-4501. Values are given in mGy/MBq for Beta, Gamma and
Total, whereas ICRP-103 ED and Effective dose is given in mSv/MBq.

| Target Organ | Alpha | Beta | Gamma | Total | ICRP-103 ED |
|---|---|---|---|---|---|
| Adrenals | 0.00E+00 | 4.33E−03 | 6.62E−02 | 7.05E−02 | 6.51E−04 |
| Brain | 0.00E+00 | 4.27E−03 | 1.82E−02 | 2.25E−02 | 2.28E−04 |
| Breasts | 0.00E+00 | 4.27E−03 | 1.76E−02 | 2.19E−02 | 2.62E−03 |
| Esophagus | 0.00E+00 | 4.27E−03 | 2.92E−02 | 3.35E−02 | 1.34E−03 |
| Eyes | 0.00E+00 | 4.27E−03 | 1.82E−02 | 2.25E−02 | 0.00E+00 |
| Gallbladder Wall | 0.00E+00 | 6.31E−03 | 4.37E−02 | 5.00E−02 | 4.61E−04 |
| Left colon | 0.00E+00 | 1.08E−02 | 5.81E−02 | 6.89E−02 | 3.34E−03 |
| Small Intestine | 0.00E+00 | 3.57E−02 | 7.07E−02 | 1.06E−01 | 9.82E−04 |
| Stomach Wall | 0.00E+00 | 1.28E−01 | 2.40E−02 | 3.69E−01 | 4.43E−02 |
| Right colon | 0.00E+00 | 1.08E−02 | 4.20E−02 | 5.28E−02 | 2.56E−03 |
| Rectum | 0.00E+00 | 1.08E−02 | 8.35E−02 | 9.43E−02 | 2.17E−08 |
| Heart Wall | 0.00E+00 | 4.78E−03 | 3.42E−02 | 3.90E−02 | 3.60E−04 |
| Kidneys | 0.00E+00 | 1.43E−02 | 4.92E−02 | 6.35E−02 | 5.86E−04 |
| Liver | 0.00E+00 | 4.06E−03 | 3.39E−02 | 3.80E−02 | 1.52E−03 |
| Lungs | 0.00E+00 | 8.84E−04 | 2.96E−02 | 3.05E−02 | 3.66E−03 |
| Ovaries | 0.00E+00 | 1.59E−03 | 5.23E−02 | 5.39E−02 | 2.16E−03 |
| Pancreas | 0.00E+00 | 4.28E−03 | 8.23E−02 | 8.66E−02 | 8.00E−04 |
| Salivary Glands | 0.00E+00 | 4.27E−03 | 1.94E−02 | 2.37E−02 | 2.37E−04 |
| Red Marrow | 0.00E+00 | 3.87E−03 | 2.77E−02 | 3.16E−02 | 3.79E−03 |
| Osteogenic Cells | 0.00E+00 | 4.41E−03 | 4.15E−02 | 4.59E−02 | 4.59E−04 |
| Spleen | 0.00E+00 | 4.29E−03 | 8.02E−02 | 8.45E−02 | 7.80E−04 |
| Thymus | 0.00E+00 | 4.28E−03 | 2.31E−02 | 2.74E−02 | 2.53E−04 |
| Thyroid | 0.00E+00 | 4.27E−03 | 1.96E−02 | 2.38E−02 | 9.53E−04 |
| Urinary Bladder Wall | 0.00E+00 | 1.31E−01 | 1.56E−01 | 2.87E−01 | 1.15E−02 |
| Uterus | 0.00E+00 | 4.27E−03 | 8.52E−02 | 8.95E−02 | 4.13E−04 |
| Total Body | 0.00E+00 | 6.46E−03 | 2.92E−02 | 3.57E−02 | 0.00E+00 |
| Effective Dose | 8.61E−08 | | | | |

TABLE 34
Calculated effective dose from administration of [¹¹¹In]In-DPI-445

| | Dose (mSv/MBq) |
|---|---|
| Effective dose to ICRP89 male | 8.68E−02 |
| phantom | |
| Effective eose to ICRP89 female | 1.19E−01 |
| phantom | |
| Average effective dose | 1.03E−01 |

TABLE 35
Calculated effective dose from administration
of [¹¹¹In]In-DPI-4501

| | Dose (mSv/MBq) |
|---|---|
| Effective Dose to ICRP89 male | 8.27E−02 |
| phantom | |
| Effective Dose to ICRP89 female | 8.61E−02 |
| phantom | |
| Average Effective dose | 8.44E−02 |

The estimated residence times for the ¹⁷⁷Lu-labelled DPI-4452 and DPI-4501 are presented in Table 36, Table 37, Table 38 and Table 39. Extrapolated Lu-177 radiation doses absorbed in human organs are presented in Table 40 and Table 41, for [¹⁷⁷Lu]Lu-DPI-4452, and in Table 42 and Table 43, for [¹⁷⁷Lu]Lu-DPI-4501. Estimates of the maximum allowed radioactivity dose in every organ according to tolerable limits set for external radiation beam therapy (taken by default as a conservative approach) are presented in Table 44 (for [¹⁷⁷Lu]Lu-DPI-4452) and Table 45 (for [¹⁷⁷Lu]Lu-DPI-4501).

TABLE 36
Input parameters for the male dose
calculation for [¹⁷⁷Lu]Lu-DPI-4452.

| Organ | Mass(g) | Residence time (h) |
|---|---|---|
| Adrenals | 14 | 0.00E+00 |
| Brain | 1450 | 0.00E+00 |
| Esophagus | 40 | 0.00E+00 |
| Eyes | 15 | 0.00E+00 |
| Gallbladder Contents | 58 | 9.76E−02 |
| Left colon | 75 | 1.95E−01 |
| Small Intestine | 350 | 3.95E+00 |
| Stomach Contents | 250 | 3.06E+00 |
| Right colon | 150 | 3.90E−01 |
| Rectum | 75 | 1.95E−01 |
| Heart Contents | 310 | 2.43E−02 |
| Heart Wall | 330 | 0.00E+00 |
| Kidneys | 310 | 1.15E+00 |
| Liver | 1800 | 3.03E+00 |
| Lungs | 1200 | 5.38E−01 |
| Pancreas | 140 | 0.00E+00 |
| Prostate | 17 | 0.00E+00 |
| Salivary Glands | 85 | 0.00E+00 |
| Red Marrow | 1170 | 1.59E−01 |
| Cortical Bone | 4400 | 0.00E+00 |
| Trabecular Bone | 1100 | 0.00E+00 |
| Spleen | 150 | 0.00E+00 |
| Testes | 35 | 5.84E−03 |
| Thymus | 25 | 0.00E+00 |
| Thyroid | 20 | 0.00E+00 |
| Urinary Bladder Contents | 211 | 1.13E+00 |
| Total Body | 73000 | 9.00E+01 |

TABLE 37
Extrapolated input parameters for the female
dose calculation for [¹⁷⁷Lu]Lu-DPI-4452

| Organ | Mass(g) | Residence time (h) |
|---|---|---|
| Adrenals | 13 | 0.00E+00 |
| Brain | 1300 | 0.00E+00 |
| Breasts | 500 | 0.00E+00 |
| Esophagus | 35 | 0.00E+00 |
| Eyes | 15 | 0.00E+00 |
| Gallbladder Contents | 48 | 1.37E−01 |
| Left colon | 80 | 3.35E−01 |
| Small Intestine | 280 | 5.74E+00 |
| Stomach Contents | 230 | 6.63E+00 |
| Right colon | 160 | 6.69E−01 |
| Rectum | 80 | 3.35E−01 |
| Heart Contents | 370 | 2.44E−02 |
| Heart Wall | 250 | 0.00E+00 |
| Kidneys | 275.5 | 1.15E+00 |
| Liver | 1400 | 3.99E+00 |
| Lungs | 950 | 7.53E−01 |
| Ovaries | 11 | 8.09E−03 |
| Pancreas | 120 | 0.00E+00 |
| Salivary Glands | 70 | 0.00E+00 |
| Red Marrow | 900 | 1.84E−01 |
| Cortical Bone | 3200 | 0.00E+00 |
| Trabecular Bone | 800 | 0.00E+00 |
| Spleen | 130 | 0.00E+00 |
| Thymus | 20 | 0.00E+00 |
| Thyroid | 17 | 0.00E+00 |
| Urinary Bladder Contents | 160 | 1.52E+00 |
| Uterus | 80 | 0.00E+00 |
| Total Body | 60000 | 5.94E+01 |

TABLE 38
Input parameters for the male dose
calculation for [¹⁷⁷Lu]Lu-DPI-4501

| | | Kinetics Value |
|---|---|---|
| Source Organ Name | Mass [g] | [MBq-h/MBq] |
| Adrenals | 14 | 0.00E+00 |
| Brain | 1450 | 0.00E+00 |
| Esophagus | 40 | 0.00E+00 |
| Eyes | 15 | 0.00E+00 |
| Gallbladder Contents | 58 | 3.30E−02 |
| Left colon | 75 | 1.40E−01 |
| Small Intestine | 350 | 2.10E+00 |
| Stomach Cont | 250 | 7.22E+00 |
| Right colon | 150 | 2.79E−01 |
| Rectum | 75 | 1.40E−01 |
| Heart Contents | 510 | 3.23E−02 |
| Heart Wall | 330 | 0.00E+00 |
| Kidneys | 310 | 6.12E−01 |
| Liver | 1800 | 1.03E+00 |
| Lungs | 1200 | 8.51E−02 |
| Pancreas | 140 | 0.00E+00 |
| Prostate | 17 | 0.00E+00 |
| Salivary Glands | 85 | 0.00E+00 |
| Red Marrow | 1170 | 5.02E−02 |
| Cortical Bone | 4400 | 0.00E+00 |
| Trabecular Bone | 1100 | 0.00E+00 |
| Spleen | 150 | 0.00E+00 |
| Testes | 35 | 1.97E−03 |
| Thymus | 25 | 0.00E+00 |
| Thyroid | 20 | 0.00E+00 |
| Urinary Bladder Contents | 211 | 1.66E+00 |
| Total Body | 73000 | 3.45E+01 |

TABLE 39
Input parameters for the female dose
calculation for [¹⁷⁷Lu]Lu-DPI-4501

| | | Kinetics Value |
|---|---|---|
| Source Organ Name | Mass [g] | [MBq-h/MBq] |
| Adrenal | 13 | 0.00E+00 |
| Brain | 1300 | 0.00E+00 |
| Breasts | 500 | 0.00E+00 |
| Esophagus | 35 | 0.00E+00 |
| Eyes | 15 | 0.00E+00 |
| Gallbladder Contents | 48 | 2.17E−02 |
| Left colon | 80 | 1.07E−01 |
| Sma Intestir | 280 | 1.34E+00 |
| Stomach Contents | 230 | 7.19E+00 |
| Right colon | 160 | 2.14E−01 |
| Rectum | 80 | 1.07E−01 |
| Heart Contents | 370 | 2.31E−02 |
| Heart Wall | 250 | 0.00E+00 |
| Kidneys | 275.5 | 3.95E−01 |
| Liver | 1400 | 6.34E−01 |
| Lungs | 950 | 8.76E−02 |
| Ovaries | 11 | 1.92E−03 |
| Pancreas | 120 | 0.00E+00 |
| Salivary Glands | 20 | 0.00E+00 |
| Red Marrow | 800 | 4.45E−02 |
| Cortical Bone | 3200 | 0.00E+00 |
| Trabecular Bone | 800 | 0.00E+00 |
| Spleen | 130 | 0.00E+00 |
| Thymus | 20 | 0.00E+00 |
| Thyroid | 17 | 0.00E+00 |
| Urinary Bladder Contents | 160 | 2.06E+00 |
| Uterus | 80 | 0.00E+00 |
| Total Body | 60000 | 2.86E+01 |

TABLE 40
Extrapolated calculated OLINDA output data for the human male
ICRP-89 phantom per MBq administered [¹⁷⁷Lu]Lu-DPI-4452.
Values are given in mGy/MBq for Beta, Gamma and Total, whereas
ICRP-103 ED and effective dose is given in mSv/MBq.

| Target Organ | Alpha | Beta | Gamma | Total | ICRP-103 ED |
|---|---|---|---|---|---|
| Adrenals | 0.00E+00 | 1.07E−01 | 1.75E−02 | 1.25E−01 | 1.15E−03 |
| Brain | 0.00E+00 | 1.05E−01 | 1.05E−01 | 1.14E−01 | 1.14E−03 |
| Esophagus | 0.00E+00 | 1.05E−01 | 1.23E−02 | 1.17E−01 | 4.70E−03 |
| Eyes | 0.00E+00 | 1.05E−01 | 8.60E−03 | 1.14E−01 | 0.00E+00 |
| Gallbladder Wall | 0.00E+00 | 1.77E−01 | 1.67E−02 | 1.94E−01 | 1.79E−03 |
| Left colon | 0.00E+00 | 2.16E−01 | 1.90E−02 | 2.35E−01 | 1.14E−02 |
| Small Intestine | 0.00E+00 | 5.86E−01 | 2.29E−02 | 6.09E−01 | 5.62E−03 |
| Stomach Wall | 0.00E+00 | 6.28E−01 | 2.60E−02 | 6.54E−01 | 7.85E−02 |
| Right colon | 0.00E+00 | 2.16E−01 | 1.65E−02 | 2.33E−01 | 1.13E−02 |
| Rectum | 0.00E+00 | 2.16E−01 | 1.64E−02 | 2.33E−01 | 5.35E−03 |
| Heart Wall | 0.00E+00 | 1.07E−01 | 1.19E−02 | 1.19E−01 | 1.10E−03 |
| Kidneys | 0.00E+00 | 3.16E−01 | 1.73E−02 | 3.33E−01 | 3.07E−03 |
| Liver | 0.00E+00 | 1.43E−01 | 1.42E−02 | 1.57E−01 | 6.30E−03 |
| Lungs | 0.00E+00 | 3.80E−02 | 1.03E−02 | 4.83E−02 | 5.80E−03 |
| Pancreas | 0.00E+00 | 1.05E−01 | 2.29E−02 | 1.28E−01 | 1.18E−03 |
| Prostate | 0.00E+00 | 1.05E−01 | 1.52E−02 | 1.20E−01 | 5.56E−04 |
| Salivary Glands | 0.00E+00 | 1.05E−01 | 1.09E−02 | 1.16E−01 | 1.16E−03 |
| Red Marrow | 0.00E+00 | 8.55E−02 | 1.06E−02 | 9.61E−02 | 1.15E−02 |
| Osteogenic Cells | 0.00E+00 | 1.15E−01 | 2.02E−02 | 1.35E−01 | 1.35E−03 |
| Spleen | 0.00E+00 | 1.05E−01 | 1.52E−02 | 1.20E−01 | 1.11E−03 |
| Testes | 0.00E+00 | 1.41E−02 | 8.69E−03 | 2.28E−02 | 9.11E−04 |
| Thymus | 0.00E+00 | 1.05E−01 | 9.92E−03 | 1.15E−01 | 1.06E−03 |
| Thyroid | 0.00E+00 | 1.05E−01 | 1.06E−02 | 1.16E−01 | 4.63E−03 |
| Urinary Bladder Wall | 0.00E+00 | 3.34E−01 | 1.87E−02 | 3.53E−01 | 1.41E−02 |
| Total Body | 0.00E+00 | 1.21E−01 | 1.07E−02 | 1.32E−01 | 0.00E+00 |
| Effective Dose | 1.75E−01 | | | | |

TABLE 41
Extrapolated calculated OLINDA output data for the human female
ICRP-89 phantom per MBq administered [¹⁷⁷Lu]Lu-DPI-4452.
Values are given in mGy/MBq for Beta, Gamma and Total, whereas
ICRP-103 ED and effective dose is given in mSv/MBq.

| | | | | | ICRP- |
|---|---|---|---|---|---|
| Target Organ | Alpha | Beta | Gamma | Total | 103 ED |
| Adrenals | 0.00E+00 | 8.60E−02 | 2.31E−02 | 1.09E−01 | 1.01E−03 |
| Brain | 0.00E+00 | 8.45E−02 | 7.05E−03 | 9.15E−02 | 9.15E−04 |
| Breast | 0.00E+00 | 8.45E−02 | 6.45E−03 | 9.09E−02 | 1.09E−02 |
| Esophagus | 0.00E+00 | 8.45E−02 | 1.08E−02 | 9.53E−02 | 3.81E−03 |
| Eyes | 0.00E+00 | 8.45E−02 | 7.05E−03 | 9.15E−02 | 0.00E+00 |
| Gallbladder | 0.00E+00 | 2.06E−01 | 2.16E−02 | 2.28E−01 | 2.10E−03 |
| Wall | | | | | |
| Left colon | 0.00E+00 | 2.63E−01 | 2.02E−02 | 2.83E−01 | 1.37E−02 |
| Small | 0.00E+00 | 9.59E−01 | 2.79E−02 | 9.87E−01 | 9.11E−03 |
| Intestine | | | | | |
| Stomach | 0.00E+00 | 1.31E+00 | 4.84E−02 | 1.36E+00 | 1.64E−01 |
| Wall | | | | | |
| Right colon | 0.00E+00 | 2.63E−01 | 1.84E−02 | 2.81E−01 | 1.36E−02 |
| Rectum | 0.00E+00 | 2.63E−01 | 1.73E−02 | 2.80E−01 | 6.44E−03 |
| Heart Wall | 0.00E+00 | 8.74E−02 | 1.22E−02 | 9.96E−02 | 9.19E−04 |
| Kidneys | 0.00E+00 | 3.54E−01 | 2.00E−02 | 3.74E−01 | 3.45E−03 |
| Liver | 0.00E+00 | 2.43E−01 | 1.90E−02 | 2.62E−01 | 1.05E−02 |
| Lungs | 0.00E+00 | 6.72E−02 | 1.15E−02 | 7.87E−02 | 9.44E−03 |
| Ovaries | 0.00E+00 | 6.19E−02 | 1.59E−02 | 7.78E−02 | 3.11E−03 |
| Pancreas | 0.00E+00 | 8.48E−02 | 2.75E−02 | 1.12E−01 | 1.04E−03 |
| Salivary | 0.00E+00 | 8.45E−02 | 7.40E−03 | 9.19E−02 | 9.19E−04 |
| Glands | | | | | |
| Red Marrow | 0.00E+00 | 7.24E−02 | 1.01E−02 | 8.25E−02 | 9.90E−03 |
| Osteogenic | 0.00E+00 | 6.97E−02 | 1.79E−02 | 8.76E−02 | 8.76E−04 |
| Cells | | | | | |
| Spleen | 0.00E+00 | 8.48E−02 | 2.27E−02 | 1.07E−01 | 9.92E−04 |
| Thymus | 0.00E+00 | 8.48E−02 | 9.33E−03 | 9.41E−02 | 8.69E−04 |
| Thyroid | 0.00E+00 | 8.45E−02 | 7.58E−03 | 9.21E−02 | 3.68E−03 |
| Urinary | 0.00E+00 | 4.91E−01 | 1.90E−02 | 5.10E−01 | 2.04E−02 |
| Bladder Wall | | | | | |
| Uterus | 0.00E+00 | 8.45E−02 | 1.90E−02 | 1.04E−01 | 4.78E−04 |
| Total Body | 0.00E+00 | 1.15E−01 | 1.05E−02 | 1.26E−01 | 0.00E+00 |
| Effective | 2.92E−01 | | | | |
| Dose | | | | | |

TABLE 41b
Extrapolated calculated effective dose from
administration of [¹⁷⁷Lu]Lu-DPI-4452

| | Dose (mSv/MBq) |
|---|---|
| Effective Dose to ICRP89 male phantom | 1.75E−01 |
| Effective Dose to ICRP89 female phantom | 2.92E−01 |
| Average Effective dose | 2.34E−01 |

TABLE 42
Extrapolated calculated OLINDA output data for the human male
ICRP-89 phantom per MBq administered [¹⁷⁷Lu]Lu-DPI-4501.
Values are given in mGy/MBq for Beta, Gamma and Total, whereas
ICRP-103 ED and effective dose is given in mSv/MBq.

| | | | | | ICRP- |
|---|---|---|---|---|---|
| Target Organ | Alpha | Beta | Gamma | Total | 103 ED |
| Adrenals | 0.00E+00 | 4.13E−02 | 1.08E−02 | 5.20E−02 | 4.80E−04 |
| Brain | 0.00E+00 | 4.03E−02 | 3.31E−03 | 4.36E−02 | 4.36E−04 |
| Esophagus | 0.00E+00 | 4.03E−02 | 9.27E−03 | 4.96E−02 | 1.98E−03 |
| Eyes | 0.00E+00 | 4.03E−02 | 3.32E−03 | 4.36E−02 | 0.00E+00 |
| Gallbladder | 0.00E+00 | 6.48E−02 | 7.83E−03 | 7.26E−02 | 6.70E−04 |
| Wall | | | | | |
| Left colon | 0.00E+00 | 1.20E−01 | 1.19E−02 | 1.32E−01 | 6.38E−03 |
| Small | 0.00E+00 | 2.96E−01 | 1.21E−02 | 3.08E−01 | 2.85E−03 |
| Intestine | | | | | |
| Stomach | 0.00E+00 | 1.27E+00 | 4.05E−02 | 1.31E+00 | 1.58E−01 |
| Wall | | | | | |
| Right colon | 0.00E+00 | 1.20E−01 | 7.73E−03 | 1.27E−01 | 6.18E−03 |
| Rectum | 0.00E+00 | 1.20E−01 | 8.29E−03 | 1.28E−01 | 2.94E−03 |
| Heart Wall | 0.00E+00 | 4.30E−02 | 8.92E−03 | 5.19E−02 | 4.79E−04 |
| Kidneys | 0.00E+00 | 1.68E−01 | 9.12E−03 | 1.77E−01 | 1.63E−03 |
| Liver | 0.00E+00 | 4.85E−02 | 7.17E−03 | 5.56E−02 | 2.23E−03 |
| Lungs | 0.00E+00 | 6.04E−03 | 6.28E−03 | 1.23E−02 | 1.48E−03 |
| Ovaries | 0.00E+00 | 4.03E−02 | 2.29E−02 | 6.32E−02 | 5.83E−04 |
| Pancreas | 0.00E+00 | 4.03E−02 | 7.83E−03 | 4.81E−02 | 2.22E−04 |
| Salivary | 0.00E+00 | 4.03E−02 | 4.23E−03 | 4.45E−02 | 4.45E−04 |
| Glands | | | | | |
| Red Marrow | 0.00E+00 | 3.23E−02 | 5.04E−03 | 3.74E−02 | 4.48E−03 |
| Osteogenic | 0.00E+00 | 4.37E−02 | 9.04E−03 | 5.27E−02 | 5.27E−04 |
| Cells | | | | | |
| Spleen | 0.00E+00 | 4.04E−02 | 1.16E−02 | 5.19E−02 | 4.79E−04 |
| Testes | 0.00E+00 | 4.75E−03 | 3.69E−03 | 8.44E−03 | 3.38E−04 |
| Thymus | 0.00E+00 | 4.03E−02 | 4.70E−03 | 4.50E−02 | 4.15E−04 |
| Thyroid | 0.00E+00 | 4.03E−02 | 4.38E−03 | 4.47E−02 | 1.79E−03 |
| Urinary | 0.00E+00 | 3.76E−01 | 1.47E−02 | 3.90E−01 | 1.56E−02 |
| Bladder Wall | | | | | |
| Total Body | 0.00E+00 | 5.59E−02 | 4.69E−03 | 6.06E−02 | 0.00E+00 |
| Effective | 2.10E−01 | | | | |
| Dose | | | | | |

TABLE 43a
Extrapolated calculated OLINDA output data for the human female
ICRP-89 phantom per MBq administered[¹⁷⁷Lu]Lu-DPI-4501.
Values are given in mGy/MBq for Beta, Gamma and Total, whereas
ICRP-103 ED and effective dose is given in mSv/MBq.

| | | | | | ICRP- |
|---|---|---|---|---|---|
| Target Organ | Alpha | Beta | Gamma | Total | 103 ED |
| Adrenals | 0.00E+00 | 4.11E−02 | 1.35E−02 | 5.46E−02 | 5.04E−04 |
| Brain | 0.00E+00 | 4.07E−02 | 3.39E−03 | 4.41E−02 | 4.41E−04 |
| Breast | 0.00E+00 | 4.07E−02 | 3.28E−03 | 4.40E−02 | 5.27E−03 |
| Esophagus | 0.00E+00 | 4.07E−02 | 5.84E−03 | 4.65E−02 | 1.86E−03 |
| Eyes | 0.00E+00 | 4.07E−02 | 3.39E−03 | 4.41E−02 | 0.00E+00 |
| Gallbladder | 0.00E+00 | 6.00E−02 | 8.13E−03 | 6.81E−02 | 6.29E−04 |
| Wall | | | | | |
| Left colon | 0.00E+00 | 9.77E−02 | 1.08E−02 | 1.09E−01 | 5.26E−03 |
| Small | 0.00E+00 | 2.44E−01 | 1.14E−02 | 2.55E−01 | 2.36E−03 |
| Intestine | | | | | |
| Stomach | 0.00E+00 | 1.37E+00 | 4.44E−02 | 1.42E+00 | 1.70E−01 |
| Wall | | | | | |
| Right colon | 0.00E+00 | 9.77E−02 | 7.30E−03 | 1.05E−01 | 5.09E−03 |
| Rectum | 0.00E+00 | 9.77E−02 | 1.03E−02 | 1.08E−01 | 2.48E−03 |
| Heart Wall | 0.00E+00 | 4.33E−02 | 7.04E−03 | 5.04E−02 | 4.65E−04 |
| Kidneys | 0.00E+00 | 1.22E−01 | 8.86E−03 | 1.30E−01 | 1.20E−03 |
| Liver | 0.00E+00 | 3.85E−02 | 6.62E−03 | 4.51E−02 | 1.81E−03 |
| Lungs | 0.00E+00 | 7.83E−03 | 5.98E−03 | 1.38E−02 | 1.66E−03 |
| Ovaries | 0.00E+00 | 1.47E−02 | 7.94E−03 | 2.26E−02 | 9.05E−04 |
| Pancreas | 0.00E+00 | 4.07E−02 | 1.68E−02 | 5.75E−02 | 5.31E−04 |
| Salivary | 0.00E+00 | 4.07E−02 | 3.57E−03 | 4.42E−02 | 4.42E−04 |
| Glands | | | | | |
| Red Marrow | 0.00E+00 | 3.25E−02 | 5.02E−03 | 3.75E−02 | 4.50E−03 |
| Osteogenic | 0.00E+00 | 3.27E−02 | 8.82E−03 | 4.15E−02 | 4.15E−04 |
| Cells | | | | | |
| Spleen | 0.00E+00 | 4.08E−02 | 1.65E−02 | 5.72E−03 | 5.28E−04 |
| Thymus | 0.00E+00 | 4.07E−02 | 4.40E−03 | 4.51E−02 | 4.16E−04 |
| Thyroid | 0.00E+00 | 4.07E−02 | 3.61E−03 | 4.43E−02 | 1.77E−03 |
| Urinary | 0.00E+00 | 5.89E−01 | 1.49E−02 | 6.04E−01 | 2.42E−02 |
| Bladder Wall | | | | | |
| Uterus | 0.00E+00 | 4.07E−02 | 1.09E−02 | 5.15E−02 | 2.38E−04 |
| Total Body | 0.00E+00 | 5.80E−02 | 5.27E−03 | 6.33E−02 | 0.00E+00 |
| Effective | 2.33E−01 | | | | |
| Dose | | | | | |

TABLE 43b
Extrapolated calculated effective dose from
administration of [¹⁷⁷Lu]Lu-DPI-4501

| | Dose (mSv/MBq) |
|---|---|
| Effective Dose to ICRP89 male phantom | 2.10E−01 |
| Effective Dose to ICRP89 female phantom | 2.33E−01 |
| Average Effective dose | 2.22E−01 |

TABLE 44
Estimate of the maximum allowed injected radioactivity dose
of [¹⁷⁷Lu]Lu-DPI-4452, based on individual organ dose
limits and the calculated absorbed radiation dose per injection/MBq.
The limiting organ is written in bold. Note that allowed
injected radioactivity dose refers to the maximum administered
radioactivity enabling the radiation dose absorbed in an
organ to remain below the given dose limit.

| | | | Maximum |
|---|---|---|---|
| | Absorbed radiation | Absorbed | allowed injected |
| | dose per injected | dose | radioactivity |
| Organ | activity (mGy/MBq) | limit (Gy) | dose (MBq) |
| Left colon | 2.35E−01 | 25 | 1.06E+05 |
| Small Intestine | 6.09E−01 | 18 | 2.96E+04 |
| Stomach Wall | 6.54E−01 | 28 | 4.28E+04 |
| Right Colon | 2.33E−01 | 25 | 1.07E+05 |
| Rectum | 2.33E−01 | 25 | 1.07E+05 |
| Heart Wall | 1.19E−01 | 32 | 2.69E+05 |
| Kidneys | 3.33E−01 | 23 | 6.91E+04 |
| Liver | 1.57E−01 | 21 | 1.34E+05 |
| Lungs | 4.83E−02 | 12 | 2.48E+05 |
| Urinary Bladder | 3.53E−01 | 18.3 | 5.18E+04 |
| Wall | | | |

TABLE 45
Estimate of the maximum allowed injected radioactivity dose
of [¹⁷⁷Lu]Lu-DPI-4501, based on individual organ dose
limits and the calculated absorbed radiation dose/per injection/MBq.
The limiting organ is written in bold. Note that allowed
injected radioactivity dose refers to the maximum administered
radioactivity enabling the radiation dose absorbed in an
organ to remain below the given dose limit.

| | | | Maximum |
|---|---|---|---|
| | Absorbed radiation | Absorbed | allowed injected |
| | dose per injected | dose | radioactivity |
| Organ | activity (mGy/MBq) | limit (Gy) | dose (MBq) |
| Left colon | 1.32E−01 | 25 | 1.89E+05 |
| Small Intestine | 3.08E−01 | 18 | 5.84E+04 |
| Stomach Wall | 1.31E+00 | 28 | 2.14E+04 |
| Right Colon | 1.27E−01 | 25 | 1.97E+05 |
| Rectum | 1.28E−01 | 25 | 1.95E+05 |
| Heart Wall | 5.19E−02 | 32 | 6.17E+05 |
| Kidneys | 1.77E−01 | 23 | 1.30E+05 |
| Liver | 5.56E−02 | 21 | 3.78E+05 |
| Lungs | 1.23E−02 | 12 | 9.76E+05 |
| Urinary Bladder | 3.90E−01 | 18.3 | 4.69E+04 |
| Wall | | | |

The above extrapolations were made under the assumption that CAIX expression levels in humans are similar to dogs.

For [¹⁷⁷Lu]Lu-DPI-4452, the dose-limiting organ appeared to be the small intestine, and the maximum allowed radioactivity dose would be 29.6 GBq. For this administered radioactivity dose, the estimated radiation dose delivered to a representative 11.0-g tumor is within the range 12.2-660 Gy (Table 46), which is compatible with antitumoral effect in humans.

TABLE 46
Tumor radiation dose at maximum injected
activity for [¹⁷⁷Lu]Lu-DPI-4452.

| | Dose at Max | Dose at Max |
|---|---|---|
| | tolerated | tolerated |
| Tumor | activity % ID | activity % ID/g |
| mass (g) | method (Gy) | method (Gy) |
| 3.00E+00 | 2.40E+03 | 1.22E+01 |
| 1.10E+01 | 6.60E+02 | 1.22E+01 |
| 3.00E+01 | 2.43E+02 | 1.23E+01 |
| 1.00E+02 | 7.34E+01 | 1.24E+01 |
| 5.94E+02 | 1.26E+01 | 1.26E+01 |

For [¹⁷⁷Lu]Lu-DPI-4501, the dose-limiting organ appeared to be the stomach wall, and the maximum allowed radioactivity dose would be 21.4 GBq. For this administered radioactivity dose, the estimated radiation dose delivered to a representative 11.0-g tumor is within the range 4.4-205 Gy (Table 47).

TABLE 47
Tumor radiation dose at maximum injected
activity for [¹⁷⁷Lu]Lu-DPI-4501.

| | Dose at Max | Dose at Max |
|---|---|---|
| | tolerated | tolerated |
| Tumor | activity % ID | activity % ID/g |
| mass (g) | method (Gy) | method (Gy) |
| 3.00E+00 | 7.46E+02 | 4.32E+00 |
| 1.10E+01 | 2.05E+02 | 4.36E+00 |
| 3.00E+01 | 7.55E+01 | 4.36E+00 |
| 1.00E+02 | 2.29E+01 | 4.40E+00 |
| 5.94E+02 | 4.47E+00 | 4.47E+00 |

### Example 28: Binding Study to Human, Dog, and Mouse CAIX

The species cross-reactivity of DPI-4452 and DPI-4501 (also referred to in the present application as “3BP-4452” and “3BP-4501”, respectively) for CAIX was investigated by measuring the equilibrium dissociation constant Kd in CHO cells transfected with human, dog or mouse CAIX in a radioligand binding assay, employing the ¹¹¹In-labeled versions of DPI-4452 and DPI-4501 at 8 different concentrations. After attainment of equilibrium, the cells were harvested, and the bound fraction of the compounds was measured. The resulting saturation binding data were analyzed using Graph Pad Prism 8.3.

CHO cells transfected with human, dog, and mouse CAIX (CHO-huCA9 T04J-1/20 K1, CHO-dogCA9 T05J-9/20 K4, CHO-murCA9 T05J-3/20 K4) were obtained from InSCREENex (Germany).

For radiolabeling, 200 M stock solutions of DPI-4452 and DPI-4501 were prepared by dissolution in 0.1 M HEPES, aliquoted and stored at −20° C. A molar excess of 3BP-3565 was used as a blocking peptide to assess non-specific binding in autoradiographic studies. 3BP-3565 binds with a similar affinity to CAIX and blocks binding sites of test compounds. For the blocking solution, a 10 mM stock solution of 3BP-3565 was prepared by dissolution in DMSO.

TABLE 48
List of test and reference compounds,
sequence formulae and metals used

| Compound | | |
|---|---|---|
| Id | Structure | Metal |
| DPI-4452 | DOTA-PPAc-Q-[C(3MeBn)-EPD-Af3(Cpsu)- | (none) |
| | LTWSC]-NH₂ | |
| [¹¹¹In]In- | ¹¹¹In-DOTA-PPAc-Q-[C(3MeBn)- | ¹¹¹In |
| DPI-4452 | EPD-Af3(Cpsu)-LTWSC]-NH₂ | |
| DPI-4501 | DOTA-Q-[C(3MeBn)-EPD-Aph(SaPr)- | (none) |
| | LTWSC]-NH₂ | |
| [¹¹¹In]In- | ¹¹¹In-DOTA-Q-[C(3MeBn)- | ¹¹¹In |
| DPI-4501 | EPD-Aph(SaPr)-LTWSC]-NH₂ | |
| 3BP-3565 | AcVY-[C(3MeBn)-EPDWLTWSC]-NH₂ | (none) |

CHO cells were maintained in Ham's medium supplemented with 10% fetal bovine serum, 2 mM L-glutamine, 100 U/mL Penicillin and 0.1 mg/mL streptomycin under standard cell culture conditions. The cells were grown in uncoated cell culture flasks (150 cm², Biochrom) to subconfluence and then split 1:2-1:3. Approximately 24 hours before the assay, cells were detached by incubation with Accutase and carefully tapping the flasks. Detached cells were resuspended in medium and collected by centrifugation (300 g, 5 min, RT). Cell pellets were resuspended in cell culture medium and counted using a particle counter (CASY Model TT, Scharfe Systems, Germany). Cell concentrations were adjusted to 3×10⁵ mL⁻¹, and 1.000 μL per well of the suspension were dispensed into poly-D-lysine-coated flat-clear-bottom 24 well plates.

Compound radiolabeling was performed as follows. At the start of synthesis, volumes of radionuclide solution (¹¹¹InCl₃ in 20 mM HCl) containing required amounts of activity were mixed with appropriate volumes of 200 M DPI-4452 and DPI-4501 stock solutions to yield a specific activity of 45 MBq/nmol. Then, 25 mg/mL methionine in 1 M sodium acetate buffer pH 5 was added at a final concentration of 0.1 M sodium acetate. Subsequently, the mixture was heated to 80° C. for 25 min, followed by cooling for 5 minutes. Finally, 20 μL of 200 mg/mL ascorbic acid solution, 2.5 μL of 5 mg/mL DTPA and 2.5 μL of 5% TWEEN-20 per 100 μL reaction mixture were added, resulting in the concentrated study solution. For quality control, an aliquot of the labeling solution was diluted 1:40 with 0.1% TWEEN-20 in 0.1 M sodium acetate buffer pH 5. Five microliters of the diluted labeling solution were injected onto a Poroshell SB-C18 2.1×50 mm, 2.7 μm column. HPLC analysis was performed as follows: Gradient A: H₂O, 0.1% TFA, Gradient B: Acetonitrile (MeCN), gradient from 5% B to 70% B within 15 min, flow rate of 0.5 mL/min; detector: NaI, DAD 215 nm. The peak eluting with the dead volume represents free radionuclide, the peak eluting with the ligand-specific retention time as determined with a non-labeled sample represents the radiolabeled compound. The radiochemical purities (RCP's) of [¹¹¹In]In-DPI-4452 and [¹¹¹In]In-DPI-4501 according to HPLC were ≥88% and ≥86%, respectively.

The assessment of the time needed until attainment of equilibration on CHO-huCAIX and CHO-dgCAIX was performed as follows. The 10 mM 3BP-3565 stock solution was diluted with assay medium (Ham's medium without additives) to prepare an 8-μM blocking working solution. The radiolabeling mixtures were diluted with assay medium to prepare 160 nM radioligand working solutions. Subsequently, 1.6 and 3.2 nM radioligand dilutions were prepared by diluting the radioligand working solutions 1:100 and 1:50, respectively, with assay medium. Approximately 24 hours after re-seeding (3×10⁵ mL⁻¹ cells per well), the medium was aspirated, and the cells were washed once with assay medium (700 μL). To determine total binding, 700 μL of binding medium and 100 μL of the radioligand dilutions were added to the wells in triplicates.

To determine non-specific binding, 600 μL of binding medium, 100 μL of the 8 M 3BP-3565 blocking solution and 100 μL of the radioligand dilutions were added to the wells in triplicates. The plates were incubated for 1 h, 3 h, 6 h, and 8 h at 37° C. under standard cell culture conditions (5% CO2). At the end of the incubation time, the plates were placed on ice while aspirating the radioligand solutions. Subsequently, the cells were washed with ice-cold PBS (0.5 mL, 1 mL, 1 mL). 300 μL of RIPA buffer containing PIC was added to each well and the plates were placed on a shaker for 10 min at ambient temperature. 200 μL of the cell lysate of each well was transferred to gamma-counting tubes (12×75 mm; e.g., VWR 212-1809 with caps 217-7004). Their associated radioactivity was counted using a gamma counter. An aliquot of each radioligand dilution was included in the gamma counter measurements to allow for determination of their actual concentrations.

The radioligand saturation binding on CHO-huCAIX and dgCAIX was determined as follows. The 10 mM 3BP-3565 stock solution was diluted with assay medium (Ham's medium without additives) to prepare a 5 M blocking working solution. The radiolabeling mixtures were diluted with assay medium to prepare 160 nM radioligand working solutions. Subsequently, following radioligand dilutions were prepared by diluting the radioligand working solutions with assay medium: i) 80 nM radioligand solution ii) 40 nM radioligand solution iii) 20 nM radioligand solution iv) 10 nM radioligand solution v) 5.0 nM radioligand solution vi) 2.5 nM radioligand solution vii) 1.3 nM radioligand solution Approximately 24 hours after re-seeding (3×10⁵ mL⁻¹ cells per well), the medium was aspirated, and the cells were washed once with assay medium (1 mL). To determine total binding, 700 μL of binding medium and 100 μL of the radioligand dilutions were added to the wells in triplicates. To determine non-specific binding, 600 μL of binding medium, 100 μL of the 5 M 3BP-3565 blocking solution and 100 μL of the radioligand dilutions were added to the wells in triplicates to determine their non-specific binding to the cells. The plates were incubated for 8 h at 37° C. under standard cell culture conditions (5% CO2). At the end of the incubation time the plates were placed on ice while aspirating the radioligand solutions. Aliquots of the 20 nM supernatants were transferred to HPLC vials for analysis of radioligand stability over the incubation time. Subsequently, the cells were washed with ice-cold PBS (0.5 mL, 1 mL, 1 mL). 300 μL of RIPA buffer containing PIC was added to each well and the plates were placed on a shaker for 10 min at ambient temperature. 200 μL of the cell lysate of each well was transferred to gamma-counting tubes. Their associated radioactivity was counted using a gamma counter and normalized to the measured protein concentration of each well (see chapter 6.3.7 BCA protein assay). An aliquot of each radioligand dilution was included in the gamma counter measurements to allow for determination of the actual radioligand concentrations in the dilution series.

The radioligand saturation binding on CHO-msCAIX was determined as follows. The 10 mM 3BP-3565 stock solution was diluted with assay medium (Ham's medium without additives) to prepare a 5 M blocking working solution. The radiolabeling mixtures were diluted with assay medium to prepare 500 nM radioligand working solutions. Subsequently, following radioligand dilutions were prepared by diluting the radioligand working solutions with assay medium: i) 250 nM radioligand solution ii) 125 nM radioligand solution iii) 63 nM radioligand solution iv) 31 nM radioligand solution v) 16 nM radioligand solution vi) 7.8 nM radioligand solution vii) 3.9 nM radioligand solution Approximately 24 hours after re-seeding (3×10⁵ mL¹ cells per well), the medium was aspirated, and the cells were washed once with assay medium (1 mL). To determine total binding, 700 μL of binding medium and 100 μL of the radioligand dilutions were added to the wells in triplicates. To determine non-specific binding, 600 μL of binding medium, 100 μL of the 5 M 3BP-3565 blocking solution and 100 μL of the radioligand dilutions were added to the wells in triplicates to determine their non-specific binding to the cells. The plates were incubated for 8 h at 37° C. under standard cell culture conditions (5% CO2). At the end of the incubation time, the plates were placed on ice while aspirating the radioligand solutions. Subsequently, the cells were washed with ice-cold PBS (0.5 mL, 1 mL, 1 mL). 300 μL of RIPA buffer containing PIC was added to each well and the plates were placed on a shaker for 10 min at ambient temperature. 200 μL of the cell lysate of each well was transferred to gamma-counting tubes. Their associated radioactivity was counted using a gamma counter and normalized to the measured protein concentration of each well. An aliquot of each radioligand dilution was included in the gamma counter measurements to allow for determination of the actual radioligand concentrations in the dilution series.

The protein concentration per well was determined via the BCA protein assay. To this end, 10 μL of each cell lysate was transferred to a 96-well microplate in duplicates before 200 μL of BCA working solution per well was added (microplate procedure according to manufacturer's instruction) and the plate was placed on a plate shaker for 30 seconds. Subsequently, the plate was incubated at 37° C. for 30 min. After cooling to ambient temperature, the absorbance at 562 nm was measured on a plate reader to determine the total protein content of each sample.

Data were analyzed using GraphPad Prism 8.3. The actual radioligand concentrations in the radioligand dilutions were calculated according to following equation:

$$c = Radioactivity concentration ( cpm / μ L ) / Specific activity ( cpm / f mol )$$

For the determination of the equilibration time, the model: Association kinetics—two or more conc. of hot. (Association kinetics at two or more concentrations of radioligand) was used that yielded the corresponding association (kₒₙ) and dissociation (k_{off}) rate constants. The equilibration time (t_{eq}) was then calculated using the following equation (Hulme et al. Br. J. Pharmacol. 2010, 161, 1219-1237):

$${t}_{eq} = 5 \times ln 2 / {k}_{off}$$

For the determination of the equilibrium dissociation constant (Kd) and the concentration of specific binding sites (Bₘₐₓ), the model: One site—Total, accounting for ligand depletion was used. The Kd provided in nM was converted into pKd (negative log of the Kd [M]). The Bmax value in cpm was converted into fmol/μg protein using the following equation:

$${B}_{max} ( f mol / μg prot ) = {B}_{max} ( cpm ) / \{ specific activity ( cpm / f mol ) \times protein content ( μg prot ) \}$$

### Results:

The stability of both test compounds during the radioligand binding assay was confirmed by HPLC analysis. To this end, aliquots of the supernatant were removed for quality control at the end of the incubation time following assay procedure.

After measurement of the dissociation rate constant k_{off} and equilibration time t_{eq} (Table 49), the incubation time for the determination of the equilibrium dissociation constant (Kd) on human, dog, and murine CAIX of [¹¹¹In]In-DPI-4452 and [¹¹¹In]In-DPI-4501 was set to be 8 h.

TABLE 49
Dissociation rate constants and calculated equilibration times

| | CHO-huCAIX | | CHO-dgCAIX | |
|---|---|---|---|---|
| | k_{off} (min⁻¹) | t_{eq} (h) | k_{off} (min⁻¹) | t_{eq} (h) |
| [¹¹¹In]In-DPI-4452 | 5.9 × 10⁻³ | 9.8 | 5.89 × 10⁻³ | 9.9 |
| [¹¹¹In]In-DPI-4501 | 7.2 × 10⁻³ | 8.0 | 9.6 × 10⁻³ | 6.0 |

For the determination of the equilibrium dissociation constants (K_{d}), the total as well as the non-specifically bound fraction of the radioligand of interest to CHO cells expressing CAIX from different species was plotted against the initial radioligand concentration. Due to [¹¹¹In]In-DPI-4501 data not showing the expected saturation binding pattern at the two highest concentrations (10 nM and 20 nM), only the lower six concentrations (0.16-5.0 nM) were used for CHO-huCAIX and CHO-dgCAIX data analysis of both test compounds. Since, The One site—Total model in GraphPad Prism, accounting for ligand depletion, was used for the analysis of all saturation binding curves since radioligand depletion in CHO-huCAIX and CHO-dgCAIX was significant (up to 64%) and provided the equilibrium dissociation constant (Kd) as well as the concentration of specific binding sites (Bₘₐₓ). Binding of the test compounds to CHO-msCAIX was low and not, or only partially blockable.

Table 50 and Table 51 summarize the equilibrium dissociation constants (pKd) as well as the concentration of specific binding sites (Bₘₐₓ) on CHO cells expressing human, dog and mouse CAIX for compounds [¹¹¹In]In-DPI-4452 and [¹¹¹In]In-DPI-4501. Two independent experiments were carried out with CHO-huCAIX und CHO-dgCAIX, and a single experiment with CHO-msCAIX.

TABLE 50
pK_{d} and Bₘₐₓ values of [¹¹¹In]In-DPI-4452

| | CHO- | CHO- | CHO- |
|---|---|---|---|
| Cell line | huCAIX | dgCAIX | msCAIX |
| i) pKd | 9.7 | 9.7 | 7.2 |
| ii) pKd | 9.3 | 9.5 | n.d. |
| Mean pKd ± SD | 9.5 ± 0.2 | 9.6 ± 0.1 | 7.2 |
| i) Bₘₐₓ [fmol/μg prot] | 12.3 | 9.0 | 5.6 |
| ii) Bₘₐₓ [fmol/μg prot] | 13.3 | 9.6 | n.d. |
| Mean Bₘₐₓ [fmol/μg prot] | 12.8 ± 0.5 | 9.3 ± 0.3 | 5.6 |

TABLE 51
pK_{d} and Bₘₐₓ values of [¹¹¹In]In-DPI-4501

| | CHO- | CHO- | CHO- |
|---|---|---|---|
| Cell line | huCAIX | dgCAIX | msCAIX |
| iii) pKd | 9.8 | 9.6 | 7.9 |
| iv) pKd | 9.4 | 9.3 | n.d. |
| Mean pKd ± SD | 9.6 ± 0.2 | 9.4 ± 0.1 | 7.9 |
| iii) Bₘₐₓ [fmol/μg prot] | 6.4 | 3.3 | 2.1 |
| iv) Bₘₐₓ [fmol/μg prot] | 8.8 | 6.1 | n.d. |
| Mean Bₘₐₓ [fmol/μg prot] | 7.6 ± 1.2 | 4.7 ± 1.4 | 2.1 |

### Conclusion:

Compounds [¹¹¹In]In-DPI-4452 and [¹¹¹In]In-DPI-4501 were found to be potent binders on human and dog CAIX expressed in CHO cells, displaying subnanomolar dissociation constants (pKd ≥9). For both compounds, the binding affinities for human and dog CAIX were comparable, qualifying dog as a suitable species for non-clinical toxicology studies, in which side effects mediated by specific and non-specific binding of a development candidate are to be assessed. In contrast, the dissociation constants with murine CAIX were approximately two magnitudes higher compared to human CAIX, disqualifying mouse as a suitable species for non-clinical toxicology studies. Moreover, binding of the test compounds to CHO-msCAIX was largely non-specific as evidenced by the nominal degree of blocking achievable via addition of an excess of non-labeled compound.

### Example 29: Dose Range Finding Study by Intravenous Route

DPI-4452 was administered by intravenous (i.v.) bolus injection to male beagle dogs at ascending dose levels of 25, 80, 400, and 800 μg/kg/day to one group of two dogs as one single-dose followed by 3 days of wash-out period.

The following parameters and endpoints were evaluated: mortality, clinical observations, body weights, food consumption, local reactions, clinical pathology parameters (hematology, coagulation, and clinical chemistry), organ weights, and macroscopic examinations. Both animals were sampled for toxicokinetics (TK) on each dosing occasion (i.e., on day 1, day 5, day 9 and day 13) at: pre-dose, 15 min, 30 min, 1 h, 6 h and 24 h. Blood samples were collected in K2EDTA tubes. Plasma was prepared by centrifugation (2500 g for 10 minutes, +4° C.) within 1 hour after collection, and then frozen within 1 hour after centrifugation and stored at −80° C. The quantification of DPI-4452 concentration in the dog plasma samples was performed using DPI-4501, an analog compound, as internal standard, and using solid-phase extraction followed by liquid chromatography—high-resolution mass spectrometry (LC-HRMS) analysis (quantification range of 2.00 ng/mL to 1000 ng/mL). Chromatographic separation was achieved using a Waters Acquity UPLC system with a Waters Acquity HSS T3 C18 2.1×50 mm, 1.8 μm column. Chromatography was conducted at 50° C. at a flow rate of 0.7 mL/min using a mobile phase consisting of A: acetonitrile, and B: 1% formic acid in water, according to the following linear gradient: 0 to 0.1 min: 90% B; 0.1 to 4.1 min: from 90% B to 74% B; 4.1 to 4.2 min: from 74% B to 5% B; 4.2 to 4.6 min: 5% B; 4.6 to 4.7 min: from 5% B to 90% B; 4.7-5.1 min; 90% B. Detection was performed using a Sciex API6600 TOF mass spectrometer. Toxicokinetic parameters were estimated from concentration-time data using Phoenix pharmacokinetic software (version 6.4, Certara L. P.). A non-compartmental approach consistent with the bolus intravenous injection was used for parameter estimation.

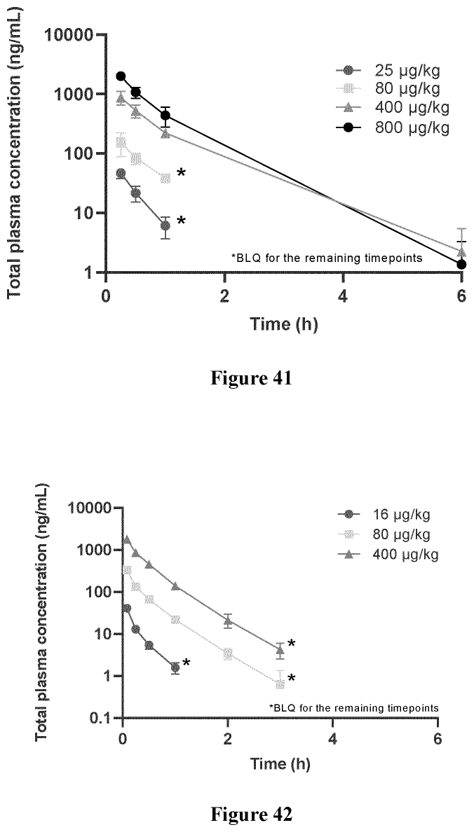

Administrations up to 800 μg/kg were well tolerated systemically and locally. Neither in-life parameters nor clinical pathology parameters were affected. In addition, there were no related macroscopic observations noted at necropsy. The TK parameters are presented in Table 52. The data suggest that exposure increased more than dose-proportionally (FIG. 41). In conclusion, doses up to 800 μg/kg/day were tolerated.

TABLE 52
Mean plasma toxicocokinetic parameters of DPI-4452 in the dog (n = 2)

| | Dose | | Dose- | | | | | |
|---|---|---|---|---|---|---|---|---|
| | level | AUCₗₐₛₜ | normalized | C_{15 min} | tₗₐₛₜ | CL | Vₛₛ | t_{1/2} |
| Period | (mg/kg) | (h × ng/mL) | AUCₗₐₛₜ | (ng/mL) | (h) | (mL/min/kg) | (L/kg) | (h) |
| Day 1 | 0.025 | 34.0 | 1.36 | 46.8 | 1; 1 | 11.7 | 0.22 | 0.25 |
| Day 5 | 0.08 | 117 | 1.46 | 156 | 1; 1 | n.a. | n.a. | n.a. |
| Day 9 | 0.4 | 926 | 2.31 | 880 | 1; 6 | 6.83 | 0.24 | 0.49 |
| Day 13 | 0.8 | 3530 | 4.42 | 2000 | 3.2; 6 | 3.52 | 0.32 | 0.88 |

AUCₗₐₛₜ = area under the plasma concentration-time curve until the last sample, Dose-normalized AUCₗₐₛₜ = reported AUCₗₐₛₜ divided by the dose level in μg/kg, CL = clearance, C_{15 min} = measured concentration at 15 min post injection, n.a. = not applicable, t_{1/2} = half-life, tₗₐₛₜ = time to last measurable concentration, Vss = volume of distribution at steady state.

### Example 30: Extended Single-Dose Toxicity Study Including Safety Pharmacology Endpoints by Intravenous Bolus Administration

In this GLP-compliant study, DPI-4452 was administered in an extended single i.v. dose in beagle dogs, including safety pharmacology endpoints at 16, 80, or 400 μg/kg in 2 subsets as described in Table 53.

TABLE 53
Experimental design of the extended single-
dose toxicity study in beagle dogs

| | | | Number and Sex of the Animals | |
|---|---|---|---|---|
| | | | Subset A: FOB and | Subset B: External |
| | | Dose | Toxicokinetics | Telemetry |
| Group | Test | Level | Early Terminal | Late Terminal |
| Number | Material | (μg/kg) | Sacrifice, Day 2 | Sacrifice, Day 16 |
| 1 | Control | 0 | 3M + 3F | 2M + 2F |
| | item | | | |
| 2 | DPI-4452 | 16 | 3M + 3F | 2M + 2F |
| 3 | DPI-4452 | 80 | 3M + 3F | 2M + 2F |
| 4 | DPI-4452 | 400 | 3M + 3F | 2M + 2F |

M = male,
F = female,
FOB = Functional Observational Battery

The following parameters and endpoints were evaluated: mortality, clinical observations, body weights, food consumption, body temperature, local reactions, ophthalmology, clinical pathology parameters (hematology, coagulation, clinical chemistry, and urinalysis), cardiovascular and respiratory safety pharmacology endpoints along with FOB evaluation, organ weights, and macroscopic and microscopic examinations, and TK parameters. The animals from subset A (3 males and 3 females per group) were sampled for toxicokinetics (TK) at: pre-dose, 5 min, 15 min, 30 min, 1 h, 2 h, 3 h and 6 h. Blood samples were collected in K2EDTA tubes. Plasma was prepared by centrifugation (2500 g for 10 minutes, +4° C.) within 1 hour after collection, and then frozen within 1 hour after centrifugation and stored at −80° C. The quantification of DPI-4452 concentration in the dog plasma samples was performed using (¹³C₆-¹⁵N)-DPI-4452 as internal standard and using solid-phase extraction followed by liquid chromatography—high-resolution mass spectrometry (LC-HRMS) analysis (lower limit of quantification: 1 ng/mL). Chromatographic separation was achieved using a Waters Acquity UPLC system with a Waters Acquity HSS T3 C18 2.1×50 mm, 1.8 μm column. Chromatography was conducted at 50° C. at a flow rate of 0.7 mL/min using a mobile phase consisting of A: 1% formic acid in acetonitrile, and B: 1% formic acid in water, according to the following linear gradient: 0 to 0.1 min: 90% B; 0.1 to 4.1 min: from 90% B to 74% B; 4.1 to 4.2 min: from 74% B to 5% B; 4.2 to 4.6 min: 5% B; 4.6 to 4.7 min: from 5% B to 90% B; 4.7-5.1 min; 90% B. Detection was performed using a Sciex API6600 TOF mass spectrometer. Toxicokinetic parameters were estimated from concentration-time data using Phoenix pharmacokinetic software (version 6.4, Certara L. P.). A non-compartmental approach consistent with the bolus intravenous injection was used for parameter estimation.

No unscheduled deaths occurred during the study, and no DPI-4452 treatment-related clinical signs or local reactions were observed. The test item treatment led to unaffected body weights, weight gains, and food intake. Likewise, no treatment-related abnormalities were reported during ophthalmic examinations, urinalysis, blood biochemistry, coagulation, and hematology investigations. There were no related organ weight changes and macroscopic and microscopic observations at early and late necropsies. Jacketed external telemetry was used to evaluate the potential cardiovascular and respiratory effects in the Subset B. Cardiorespiratory telemetry recordings were performed during the pre-test, Day 1 and 14. The electrocardiogram (ECG) parameters (heart rate, PQ interval duration, QRS complex duration, and QT interval duration) were collected continuously after dosing over 10-minute periods up to 6 h and over 15-minute periods up to 24 h. Miyazaki's QT correction method (Miyazaki, H. & Tagawa, M. Japanese Association for Laboratory Animal Science 2002, 51, 465-75) was used to correct the influence of heart rate. ECG abnormalities were checked over one minute at pre-dose, around Tmax (5, 15, and 30 min), and 24 h after the test item administration. Arterial blood pressure parameters (systolic and diastolic blood pressure and mean arterial pressure) were measured continuously during consecutive 30-minute periods at pre-dose and for up to 6 h after dosing and over consecutive 60-minute periods up to 24 h after dosing. The respiratory rate was monitored on the pre-test, Day 1 and Day 14 at 0.5, 1, 2, 3, 4, 6, 8, 12, 16, 20, and 24 h. There was no related effect on cardiovascular or respiratory parameters, including heart rate, arterial blood pressure, ECG parameters, qualitative abnormalities, or the respiratory rate at any dose or timepoints. The potential effects on central nervous system parameters were evaluated under restrained and non-restrained conditions using a FOB in Subset A. Neurologic, autonomic, and behavioral investigations were performed in the pre-test, pre-dose, and 1 hour after dosing on Day 1. No effects were observed on the central nervous system, including neurologic, autonomic, and behavioral domains.

The TK parameters of DPI-4452 are summarized in Table 54. No appreciable difference in DPI-4452 exposure was observed between male and female dogs at all dose levels. An apparent, more than dose-proportional increase in area under the plasma concentration-time curve (AUC) was observed between the low and the intermediate dose levels (at least partly explained by the fewer quantified points at the low dose), whereas a dose-proportional increase in AUC was observed between the intermediate and high dose levels (FIG. 42).

In conclusion, a single i.v. bolus injection of DPI-4452 up to 400 μg/kg was well tolerated in dogs. There were no treatment-related findings reported in clinical pathology, histopathology, FOB evaluation, and cardiovascular and respiratory safety pharmacology assessment. Based on these results, the NOAEL was considered 400 μg/kg. At the NOAEL, the mean AUCtlast values were 854 and 771 ng·h/mL for males and females, respectively. The dog's NOAEL of 400 μg/kg has a human equivalent dose of 13.3 mg, considering a 60 kg human. Since the total ligand mass dose will be 500 μg/patient, the NOAEL covers more than 25 times the predicted human dose.

TABLE 54
Summary (mean ± SD; n = 3) of toxicokinetic parameters
of DPI-4452 following a single i.v. bolus injection of DPI-
4452 at 16, 80 or 400 μg/kg in male and female beagle dogs

| | | | | | | | |
|---|---|---|---|---|---|---|---|
| | | | | AUCₗₐₛₜ/ | | | |
| | | | | Dose | | | |
| | C_{5 min} | tₗₐₛₜ* | AUCₗₐₛₜ | (h · ng/mL)/ | t_{1/2}* | CL | Vss |
| Sex | (ng/mL) | (h) | (h · ng/mL) | (μg/kg) | (h) | (mL/min/kg) | (L/kg) |

Dose level: 16 μg/kg

| | | | | | | | |
|---|---|---|---|---|---|---|---|
| Female | 37.7 | 1 | 12.4 | 0.774 | 0.239 | 21.2 | 0.30 |
| | (6.83) | [1-1] | (2.13) | (0.133) | | (4.0) | (0.06) |
| Male | 43.5 | 1 | 14.0 | 0.873 | 0.259 | 18.3 | 0.26 |
| | (2.75) | [1-1] | (1.29) | (0.0806) | | (1.9) | (0.04) |
| F/M | n.a. | n.a. | 1.13 | n.a. | n.a. | n.a. | n.a. |

Dose level: 80 μg/kg

| | | | | | | | |
|---|---|---|---|---|---|---|---|
| Female | 346 | 3 | 144 | 1.80 | 0.386 | 9.27 | 0.21 |
| | (34.8) | [2-3] | (15.3) | (0.192) | | (1.0) | (0.02) |
| Male | 332 | 2 | 132 | 1.65 | 0.361 | 10.1 | 0.21 |
| | (14.7) | [2-3] | (5.49) | (0.0687) | | (0.4) | (0.01) |
| F/M | n.a. | n.a. | 0.917 | n.a. | n.a. | n.a. | n.a. |

Dose level: 400 μg/kg

| | | | | | | | |
|---|---|---|---|---|---|---|---|
| Female | 1760 | 3 | 771 | 1.93 | 0.363 | 8.63 | 0.19 |
| | (180) | [3-3] | (40.6) | (0.101) | | (0.4) | (0.02) |
| Male | 1840 | 3 | 854 | 2.13 | 0.395 | 7.88 | 0.19 |
| | (257) | [3-3] | (120) | (0.300) | | (1.0) | (0.02) |
| F/M | n.a. | n.a. | 1.11 | NA | n.a. | n.a. | n.a. |

AUCₗₐₛₜ = area under the concentration curve to last measurable concentration, n.a. = not applicable, SD = Standard Deviation, C_{5 min} = measured concentration at 5 min post injection; F/M = Female-to-Male AUC ratio, tₗₐₛₜ = time to last measureable concentration.
*Median [Min-Max] for tₗₐₛₜ, harmonic mean for t_{1/2}.

### Example 31: Pharmacokinetic Characterization in Healthy Mice

The pharmacokinetics of DPI-4452 after a single intravenous (i.v.) dose were investigated in healthy mice. DPI-4452 was administered to male CD-1 mice via the i.v. route at 0.7 and 5.5 mg/kg. Plasma and urine samples collected after dosing were analyzed using a LC-HRMS (liquid chromatography coupled with high-resolution mass spectrometry) assay method. Concentration values were used for pharmacokinetic calculations.

DPI-4452 was formulated in 0.1 M HEPES pH 7.1 at the final concentrations of 0.35 and 2.75 mg/mL for dosing at 0.7 and 5.5 mg/kg, respectively. Male CD-1 mice (3 animals per time point and per dose level; overall, 28.2-37.6 g body weight) were administered intravenously in the tail vein in a 2 mL/kg dosing volume. Blood samples were collected in K2EDTA tubes at 5 min, 15 min, 30 min, 1 h, 2 h, 4 h, 8 h, and urine was collected over 8 hours post dose. The blood samples were kept on wet ice until plasma separation by centrifugation at room temperature; 10 min; 2500 G, which was performed within 60 min after sampling. The plasma samples were transferred into plastic tubes, frozen and stored at −80° C. until analysis.

Plasma and urine samples were analyzed by LC-HRMS after solid phase extraction, using a stable isotope-labeled internal standard (¹³C₆, ¹⁵N-DPI-4452). Chromatographic separation was run on a Waters Acquity HSS T3 C18 2.1×50 mm, 1.8 μm column. Detection was achieved on a Sciex API6600 TOF mass spectrometer. The lower limit of quantification was 1.00 ng/mL in plasma and urine.

The non-compartmental (NCA) pharmacokinetic analysis of plasma concentration data was conducted using Phoenix 64 WinNonlin (Build 8.3.3.33) software. Nominal doses were used for all animals. The terminal phase half-life (T_{1/2}) was calculated by least-squares regression analysis of the terminal linear part of the log concentration-time curve. The area under the plasma concentration-time curve (AUC) was determined with the linear trapezoidal rule for increasing values and log trapezoidal rule for decreasing values up to the last measurable concentration.

### Results:

After i.v. administration of DPI-4452 at a dose of 0.7 mg/kg, plasma concentrations declined in an apparent bi-phasic manner with a terminal elimination half-life of 0.280 h. Systemic exposure as measured by AUC_{0-inf} was 424 h*ng/mL. Clearance (CL) and volume of distribution (V_{d}) were 27.5 mL/min/kg and 0.408 L/kg, respectively. On average, 3.57% of the injected DPI-4452 dose was retrieved from urine unchanged over 8 hours.

After i.v. administration of DPI-4452 at a dose of 5.5 mg/kg, plasma concentrations declined in an apparent bi-phasic manner with a terminal elimination half-life of 0.465 h. Systemic exposure as measured by AUC_{0-inf} was 2820 h*ng/ml. Clearance (CL) and volume of distribution (V_{d}) were 32.5 ml/min/kg and 0.455 L/kg, respectively. On average, 8.89% of the injected DPI-4452 dose was retrieved from urine unchanged over 8 hours.

### Example 32: Pharmacokinetic Characterization in Healthy Dogs

The pharmacokinetics of DPI-4452 after a single intravenous (i.v.) dose was investigated in healthy dogs. DPI-4452 was administered to male beagle dogs via the i.v. route at 0.1 and 0.8 mg/kg. Plasma and urine samples collected after dosing were analyzed using a LC-HRMS (liquid chromatography coupled with high-resolution mass spectrometry) assay method. Concentration values were used for pharmacokinetic calculations.

DPI 4452 was formulated in 0.1 M HEPES pH 7.0 at the final concentrations of 0.05 and 0.4 mg/mL for dosing at 0.1 and 0.8 mg/kg, respectively. Male beagle dogs (3 animals per dose level; overall 8.9 to 12.4 kg body weight) were administered intravenously (bolus) in a 2 mL/kg dosing volume. Blood samples were collected in K₂EDTA tubes at 5 min, 15 min, 30 min, 1 h, 2 h, 4 h, 6 h, 24 h, and urine was collected over 24 hours post dose. The blood samples were kept on wet ice until plasma separation by centrifugation within 60 min after sampling. The plasma samples were transferred into plastic tubes, frozen and stored at −80° C. until analysis.

Plasma and urine samples were analyzed by LC-IRMS after solid phase extraction, using DPI-4501 as internal standard. Chromatographic separation was run on a Waters Acquity HSS T3 C18 2.1×50 mm, 1.8 μm column. Detection was achieved on a Sciex API6600 TOF mass spectrometer. The lower limit of quantification was 2.00 ng/mL in urine and plasma.

The NCA pharmacokinetic analysis of plasma concentration data was conducted using Phoenix WinNonlin 6.3 software. Nominal doses were used for all animals. The terminal phase half-life (T_{1/2}) was calculated by least-squares regression analysis of the terminal linear part of the log concentration-time curve. The area under the plasma concentration-time curve (AUC) was determined with the linear trapezoidal rule for increasing values and log trapezoidal rule for decreasing values up to the last measurable concentration.

### Results:

DPI-4452 plasma concentration was quantifiable up to 2 h and 4 h after i.v. administration at 0.1 and 0.8 mg/kg, respectively.

Systemic exposure as measured by Cₘₐₓ and AUC_{inf} after intravenous administration at 0.1 mg/kg was 377 ng/mL and 133 hr*ng/mL.

Systemic exposure as measured by Cₘₐₓ and AUC_{inf} after intravenous administration at 0.8 mg/kg was 4430 ng/mL and 1760 hr*ng/mL.

Following i.v. administration at 0.1 mg/kg, DPI-4452 showed low inter-animal variability. Clearance was 12.7 mL/min/kg and volume of distribution was 0.26 L/kg with a terminal half-life (T_{1/2}) of 0.38 h.

Following i.v. administration at 0.8 mg/kg, DPI-4452 showed low inter-animal variability. Clearance was 7.6 mL/min/kg and volume of distribution was 0.19 L/kg with a terminal half-life (T_{1/2}) of 0.48 h.

The mean amount of unchanged DPI-4452 excreted in urine after intravenous administration at a dose level of 0.1 mg/kg was 26.8 ng/mL, corresponding to 0.29% of dose excreted unchanged. This results in an estimated renal clearance of 34.4 μL/min/kg.

The mean amount of unchanged DPI-4452 excreted in urine after intravenous administration at a dose level of 0.8 mg/kg was 191 ng/mL, corresponding to 0.14% of dose excreted unchanged. This results in an estimated renal clearance of 11.4 μL/min/kg.

### Example 33: Allometry and Prediction of Human Exposure and PK Parameters

Based on available mouse and dog PK data, human PK parameters (CL, V_{d} and T_{1/2}) for DPI-4452 were predicted by allometry (Zou P et al., AAPS Journal, 14:262-281 (2012)). From the variety of CL, V_{d} and T_{1/2} determined in the animal studies, the highest and lowest values for each species were retained, and allometry was conducted on all the combinations to obtain a range for each predicted human PK parameter. The following predicted human PK parameters were obtained: clearance (CL) 3.57-13.0 mL/min/kg; volume of distribution (V_{d}) 189-455 mL/kg; terminal half-life (T_{1/2}) 0.17-1.47 h.

Simulations of human PK profiles were done with the open-source software PK Tool in order to estimate a value for C_{5 min}, the plasma concentration of DPI-4452 in humans at 5 min post dose (practical Cₘₐₓ value after i.v. dosing), after a single intravenous dose of 250 μg. The missing input parameters were dealt with as follows. Regarding blood:plasma concentration ratio, the frequent default values of 1.0 and 0.8 were considered and found to make no difference to the outputs, so it was left at 1.0. Plasma protein binding: a free fraction fᵤₚ of 1 was considered in order to estimate the highest C_{5 min} values.

The following predicted maximum human concentration was obtained: C_{5 min} of 7.91-19.0 ng/mL after an intravenous dose of 250 μg; 15.8-38.0 ng/mL after an intravenous dose of 500 μg (assuming dose-linearity between 250 and 500 μg).

### Example 34: In Vivo Efficacy of ²²⁵Ac-DPI-4452 in HT-29 (CRC) and SK-RC-52 (ccRCC) Human Cancer Cell Line Xenograft Mouse Models

The human colorectal cancer cell line HT-29 was cultured in Modified McCoy's 5a Medium supplemented with 10% FBS+1% Pen/Strep, and the human clear cell renal cancer cell line SK-RC-52 was cultured in RPMI-1640 GlutaMax-I supplemented with 10% FBS+1% Pen/Strep. 2×10⁶ cells were suspended in 100 μL PBS and Matrigel (1:1) and subcutaneously implanted into the neck of anesthetized female immunodeficient NMRI nude mice. Tumor volume (0.52×(length×width²)) and animal weight was monitored twice weekly until 42 days post treatment initiation. Animals were humanely euthanized by cervical dislocation at predefined study or humane endpoints.

Animal were randomized into equal groups based on tumor volume and body weight. Treatments were initiated at a mean group tumor volume of 140-180 mm³ and administered intravenously in the tail vein in a 100 μL dosing volume.

Treatment groups consisted of 10 mice per group for HT-29, and of 7 mice per group for SK-RC-52. For both models, treatment groups received a single administration on day 1 of either A) vehicle, B) 15 kBq of ²²⁵Ac-DPI-4452, C) 45 kBq of ²²⁵Ac-DPI-4452 or D) 135 kBq of ²²⁵Ac-DPI-4452. A satellite group of 5 mice (group E) received a single administration on day 1 of 15 kBq of ²²⁵Ac-DPI-4452 and radioactivity uptake (as % of injected dose/gram tissue) was assessed in the tumor, kidney, stomach, small intestine and colon at 4 h after administration in an automated gamma counter (Hidex Automatic Gamma Counter) for 60 seconds after reaching secular equilibrium.

For both models blood sampling for hematology was performed on all animals in group A-D at study day −1, 14 and at study end. Each mouse was restrained and 200 μL blood was obtained from the sublingual or jugular vein in EDTA-tubes and analyzed on the sampling day on a ProCyte Dx Hematology Analyzer with mouse settings.

In addition, for both models at study day 14, excess blood from the hematology analysis was spun and plasma was obtained (centrifugation at 2000 g for 10 mins at 4° C. in EDTA tubes). The plasma was analyzed for creatinine and urea concentrations using a KONELAB PRIME 30i instrument (Thermo Fisher Scientific).

All treatments with ²²⁵Ac-DPI-4452 were well tolerated in both models. No notable effect of therapy on the red blood cells and platelets levels was observed. White blood cells (WBC), lymphocytes and neutrophils showed transiently increased levels after treatment initiation for the SK-RC-52 model on study day 14, but not for the HT-29 model. For both tumor models, creatinine values were slightly elevated for animals dosed with ²²⁵Ac-DPI-4452 (135 kBq) when compared to animals dosed with vehicle. No differences in urea levels between the treatment groups were observed for both tumor models.

For the HT-29 model, animals treated with 45 kBq ²²⁵Ac-DPI-4452 or 135 kBq ²²⁵Ac-DPI-4452 had a significantly lower tumor volume compared to the vehicle control group at study day 14 (p<0.05, ordinary one-way ANOVA, corrected for multiple comparisons using Dunnett). For the SK-RC-52 model, all treatment groups had a significantly lower tumor volume compared to vehicle at study day 14 (p<0.001, ordinary one-way ANOVA, corrected for multiple comparisons using Dunnett).

Comparisons among treatment groups in both models at study day 14 and 25 showed that animals treated with 45 kBq ²²⁵Ac-DPI-4452 or 135 kBq ²²⁵Ac-DPI-4452 had a significantly lower tumor volume compared to animals treated with 15 kBq ²²⁵Ac-DPI-4452 (One-way ANOVA, corrected for multiple comparisons using Tukey).

In the satellite animals, tumor uptake 4 hours after dosing of ²²⁵Ac-DPI-4452 (15 kBq) was almost four times higher in the SK-RC-52 tumor model (39.1±1.6% ID/g) compared to the HT-29 tumor model (11.3±1.3% ID/g). The kidney uptake was 7.9±1.3 and 4.9±0.2% ID/g for ²²⁵Ac-DPI-4452 (15 kBq) for the HT-29 and SK-RC-52 models, respectively. Colon, small intestine, and stomach showed uptake of less than 1.1% ID/g in both models.

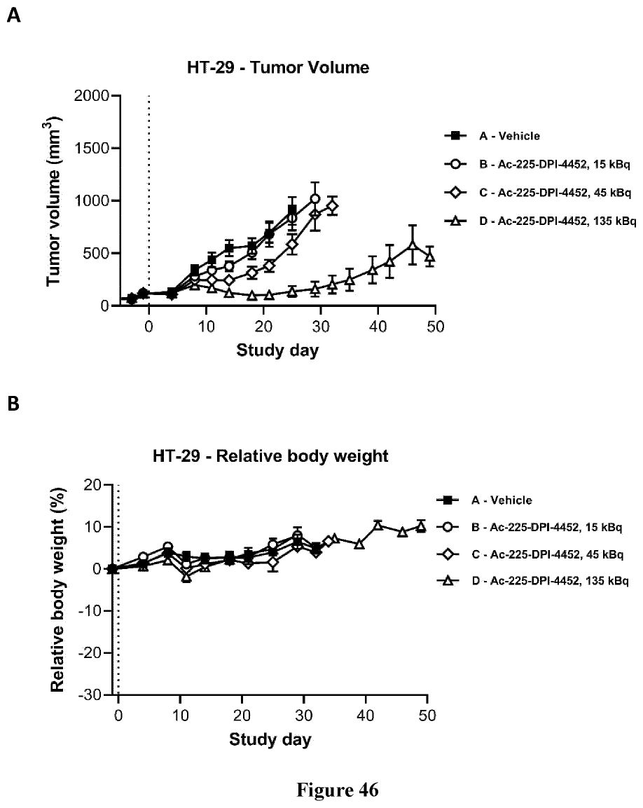

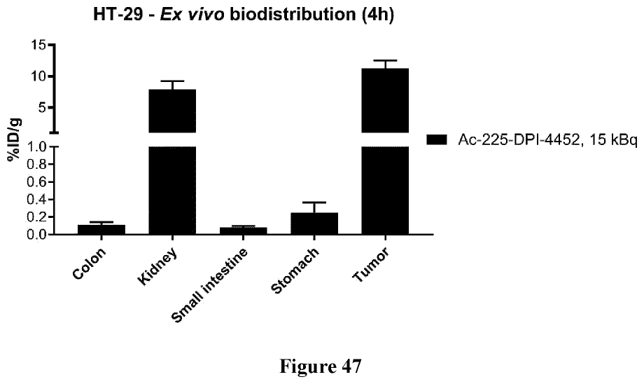

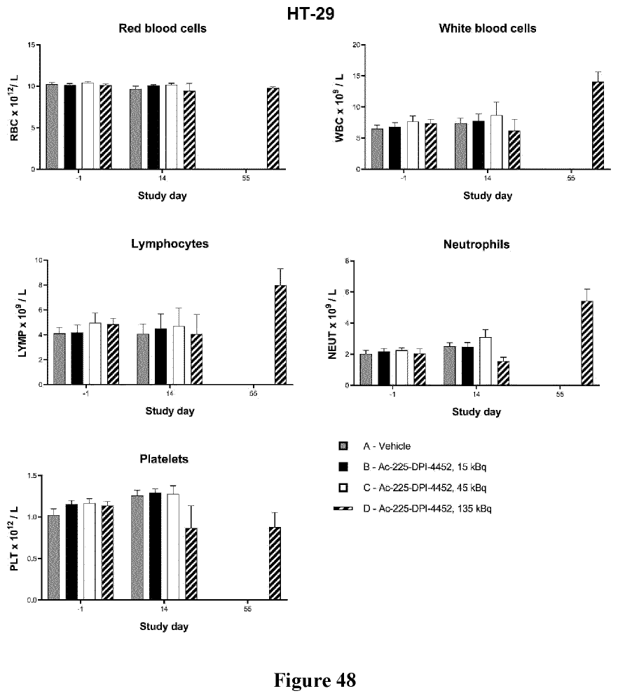

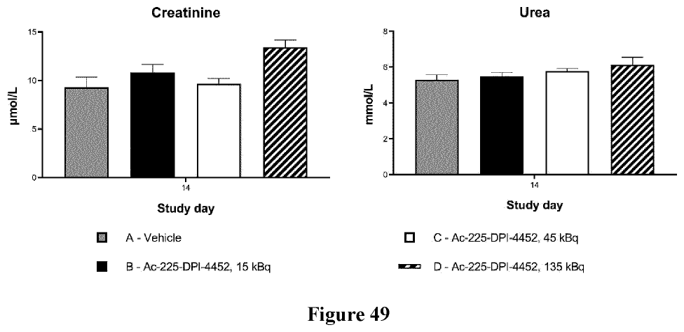

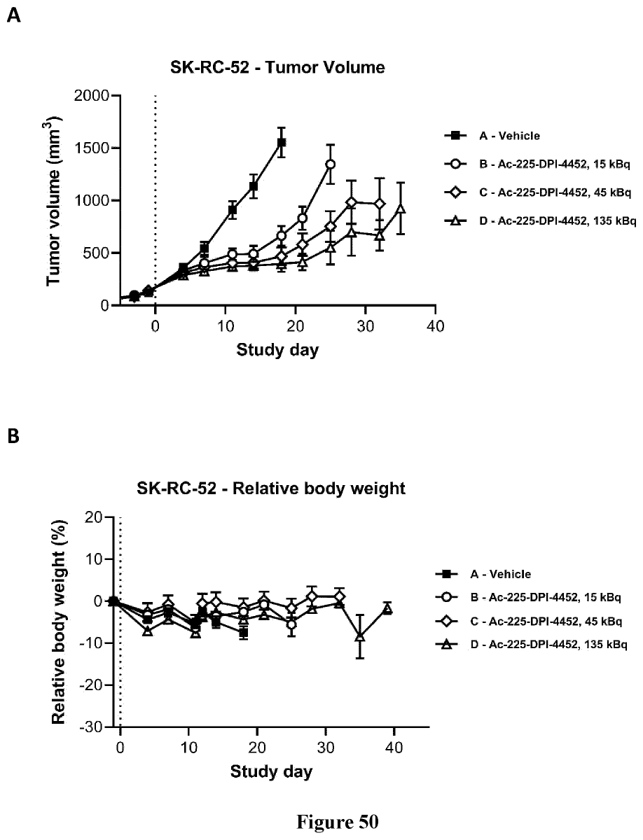

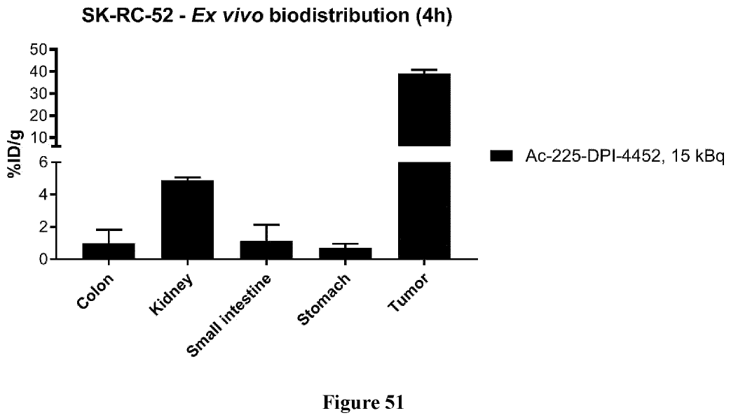

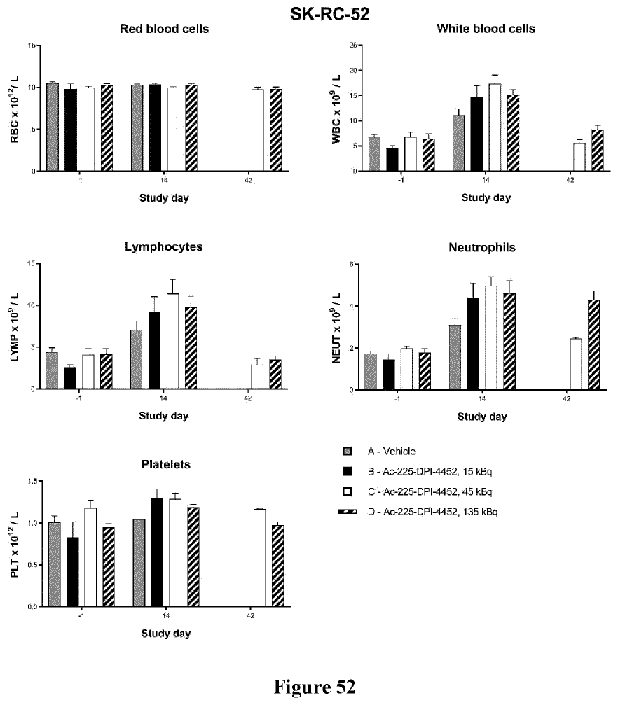

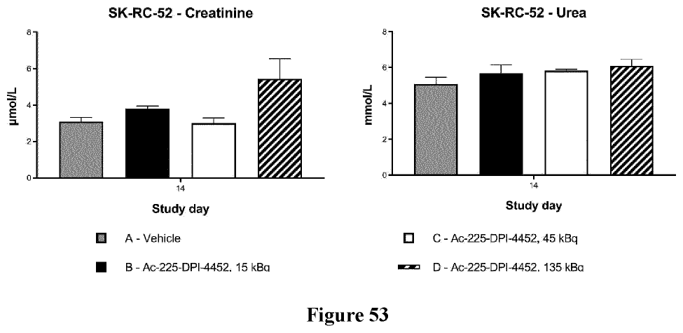

The results are shown in FIGS. 46 to 53.

The disclosure of each and any references recited herein is incorporated herein by reference.

1. A compound comprising a peptide selected from the group consisting of:
a cyclic peptide of formula (1a) 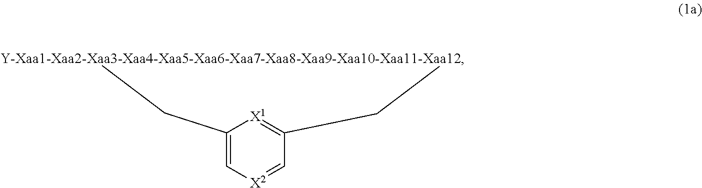
wherein, in formula (1a), the peptide sequence is drawn from left to right in N-terminal to C-terminal direction, and
Y
(iii) is Z1, wherein Z1 comprises a linker moiety L1 and an effector E1, such as a chelator, wherein the linker moiety L1 covalently links the effector E1 to Xaa1 if Xaa1 is present, or to Xaa2 if Xaa1 is absent and Xaa2 is present, or to Xaa3 if both Xaa1 and Xaa2 are absent,
or
(i) is an N-terminal modification group A selected from the group consisting of R^{0a}—SO2-, R^{0a}—CO—, R^{0a}—NH—CO—, wherein R^{0a} is selected from the group consisting of (C₁-C₁₀)alkyl, (C₅-C₁₀)aryl, and (C₁-C₅)alkyl-(C₅-C₁₀)aryl, A being preferably selected from the group consisting of 3-methyl butanoyl [Iva], Acetyl [Ac], hexanoyl [Hex], benzoyl [Bz], phenylacetyl [Pha], and propionyl [Prp],
or
(ii) comprises an effector E1, such as a chelator, wherein the effector E1 is covalently bound to Xaa1 if Xaa1 is present, or to Xaa2 if Xaa1 is absent and Xaa2 is present, or to Xaa3 if both Xaa1 and Xaa2 are absent, the effector E1 being preferably selected from the group consisting of:
(α) a moiety derived from a chromophore, wherein the chromophore is preferably selected from (α1) a phosphorophore and (α2) a fluorophore such as fluorescein or rhodamine; and
(β) a chelator optionally comprising a chelated nuclide; and
(γ) a moiety derived from a drug, preferably from a cytotoxic drug;
Xaa1 is either absent or present, and if present is a residue of an aliphatic or polar L-amino acid; preferably Xaa1 is absent or is a residue selected from the group consisting of Val, Ile, (2S)-2-amino-3,3-dimethylbutanoic acid [Tle], Ser and Thr;
Xaa2 is either present or absent, wherein
if Xaa2 is present,
(i) Xaa2 is a residue of an L-α-amino acid which is optionally N-methylated at the α-nitrogen atom,
or,
(ii) Xaa2 is a residue of an L-α-amino acid comprising, in addition to an amino group and a carboxy group attached to an α-C atom, a functional group FG1 forming a covalent linkage B1 with a functional group FG2 of Xaa11, wherein Xaa11 is a residue of an L-α-amino acid comprising, in addition to an amino group and a carboxy group attached to an α-C atom, the functional group FG2, wherein a bicyclic peptide of formula (1b) is formed: 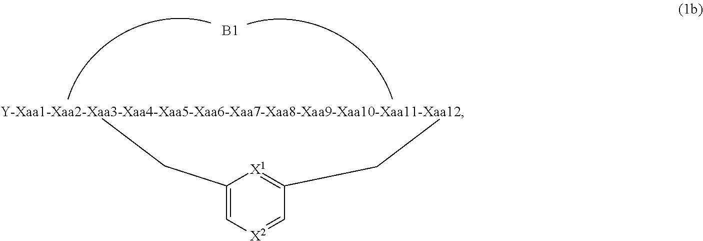
and
if Xaa2 is absent, Xaa1 is also absent;
Xaa3 is a residue of an α-amino acid of formula (X) 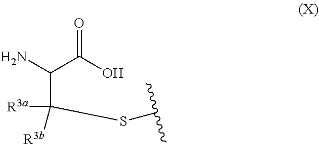
wherein
R^{3a} and R^{3b} are each and independently selected from the group consisting of H and CH₃; and
Xaa3 is preferably a residue of an L-α-amino acid such as Cys;
Xaa4 is a residue of an L-α-amino acid which is optionally N-methylated at the α-nitrogen atom;
Xaa5 is a residue of an amino acid which is optionally bound to Z3, wherein Xaa5 is a residue of an amino acid selected from the group consisting of a D-α-amino acid, N—(C₁-C₆)alkyl glycine, Gly, and an α,α-dialkylamino acid,
wherein if Xaa5 comprises Z3,
(i) Z3 is an effector E3, such as a chelator, and Xaa5 is preferably a residue of an amino acid selected from the group consisting 4-aminobutyl-glycine [Nlys], D-lys, (R)-ornithine [D-orn], (R)-2,4-diaminobutyric acid [D-dab], and (R)-2,3-diaminopropionic acid [D-dap], and the effector E3 is attached to an N atom different from the α-nitrogen atom of any one of Nlys, D-lys, D-orn, D-dab, and D-dap, or
(ii) Z3 comprises an effector E3, such as a chelator, and a linker moiety L3, Xaa5 is preferably a residue of an amino acid selected from the group consisting of Nlys, D-lys, D-orn, D-dab, and D-dap, and the linker moiety L3 is attached to an N atom different from the α-nitrogen atom of any one of Nlys, D-lys, D-orn, D-dab, and D-dap;
Xaa6 (i) is a residue of an amino acid which is selected from the group consisting of a polar L-α-amino acid, an aromatic L-α-amino acid, an aliphatic L-α-amino acid, an S-alkylated cysteine, an oxidized form of an S-alkylated cysteine, and a residue of an amino acid according to formula (3): 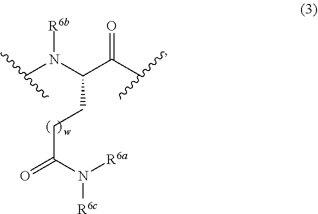
wherein
R^{6a} is selected from the group consisting of H a moiety comprising a —(C₅-C₁₀)aryl, (C₁-C₈)alkyl, and (C₁-C₅)alkyl-(C₅-C₁₀)aryl,
R^{6b} is selected from the group consisting of H or methyl,
R^{6c} is H or (C₁-C₆)alkyl, and
w is 0 or 1,
or
(ii) is a residue of an L-α-amino acid comprising, in addition to an amino group and a carboxy group attached to an α-C atom, a functional group FG3 forming a covalent linkage B2 with a functional group FG4 of Xaa11, wherein Xaa11 is a residue of an α-amino acid comprising, in addition to an amino group and a carboxy group attached to an α-C atom, the functional group FG4, wherein a bicyclic peptide of formula (1c) is formed, 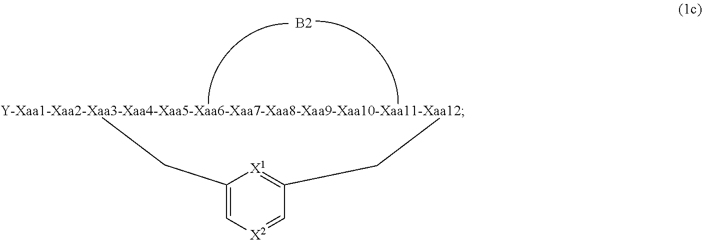
Xaa7 is a residue of an amino acid which is selected from the group consisting of a substituted aromatic amino acid, such as a substituted heteroaromatic L-α-amino acid, and an aromatic amino acid, such as a heteroaromatic L-α-amino acid;
Xaa8 is a residue of an amino acid which is selected from the group consisting of an L-α-amino acid and a cyclic α,α-dialkyl amino acid;
Xaa9 is a residue of an amino acid which is selected from the group consisting of an L-α-amino acid and Gly;
Xaa10 is a residue of a heteroaromatic L-α-amino acid;
Xaa11 (i) is a residue of an amino acid which is selected from the group consisting of an L-α-amino acid and Gly, wherein the L-α-amino acid is optionally bound to Z4, wherein Z4 comprises an effector E4, such as a chelator, and a linker moiety L4, or
(ii) is a residue of an L-α-amino acid comprising, in addition to an amino group and a carboxy group attached to an α-C atom, the functional group FG2 forming the covalent linkage B1 with the functional group FG1 of Xaa2, or
(iii) is a residue of an α-amino acid comprising, in addition to an amino group and a carboxy group attached to an α-C atom, the functional group FG4 forming the covalent linkage B2 with the functional group FG3 of Xaa6;
Xaa12 is a residue of an amino thiol of formula (XII): 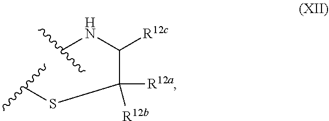
preferably of formula (XIIa): 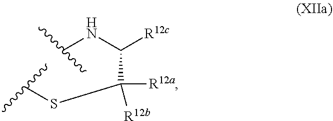
wherein
the NH of each of formulae (XII) and (XIIa) is bound to Xaa11;
R^{12a} and R^{12b} are each and independently selected from the group consisting of H and CH₃;
R^{12c} is selected from the group consisting of —CONH₂, —COOH, —CO—Z6 and —CH₂—Z6, wherein Z6 comprises a linker moiety L6 and an effector E6, such as a chelator; preferably R^{12c} is —CONH₂, and
X¹ and X² are each and independently selected from the group consisting of C—H and N, and are both preferably C—H.

2-9. (canceled)

10. The compound of claim 1, wherein the linker moiety L1 provides (a) a carboxy group forming an amide bond with an α-amino group provided by Xaa2 if Xaa1 is absent and Xaa2 is present, or with an α-amino group provided by Xaa1 if Xaa1 is present, or with an α-amino group provided by Xaa3 if both Xaa1 and Xaa2 are absent, and (b) an amino group forming a covalent bond to the effector; and wherein preferably the linker moiety L1 is a group comprising from 1 to 12 amino acids which is optionally cleavable, and/or the effector is as defined in claim 1;
wherein the linker moiety L1 is preferably selected from the group consisting of X11 and X11-X12, wherein X11 and X12 are each and individually a residue of an amino acid, wherein if the linker moiety L1 is X11, a carboxy group is provided by X11 and if the linker moiety L1 is X11-X12, a carboxy group is provided by X12, wherein the carboxy group of L1 forms an amide bond with an α-amino group provided by Xaa1 if Xaa1 is present, or with an α-amino group provided by Xaa2 if Xaa1 is absent and Xaa2 is present, or with an α-amino group provided by Xaa3 if both Xaa1 and Xaa2 are absent and X11 provides an amino group which is forming a covalent bond to the effector, and
wherein X11 and X12 are preferably each and individually a residue of an amino acid selected from the group consisting of 4-Carboxymethyl piperazine [PPac], 1,13-diamino-4,7,10-trioxatridecan-succinamic acid [Ttds], 8-amino-3,6-dioxaoctanoic acid [O2Oc], 2-(4-(amino)piperidin-1-yl)acetic acid [APac], 4-trans-aminomethylcyclohexane carboxylic acid [4Amc], and an amino acid according to any one of the following formulae (32)-(34): 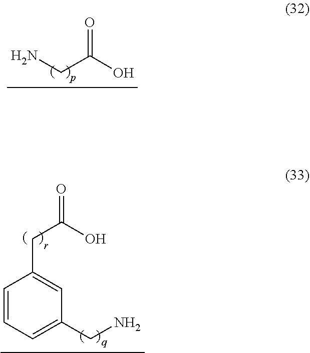
and the ortho- and para-substituted isomers thereof, and 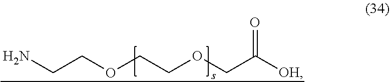
wherein
p is 2, 3, 4, 5, 6, 7, 8, 9, or 10,
q is 0, 1, 2, 3, or 4,
r is 0, 1, 2, 3, or 4,
s is 0, 1, 2, 3, 4, 5, 6, 7, 8, 9, 10, 11 or 12,
and the amino acid of formulae (32) and (33) is optionally substituted,
wherein the amino acid of formulae (32) and (33) is preferably substituted with R^{X11}—CO—NH— at an α-carbon atom which is covalently bound to the COOH-group in formulae (32) and (33), wherein R^{X11} is selected from the group consisting of (C₁-C₁₀)alkyl, (C₅-C₁₀)aryl, and (C₁-C₅)alkyl-(C₅-C₁₀)aryl, R^{X11} being preferably methyl,
wherein X11 and X12 are preferably each and individually a residue of an amino acid selected from the group consisting of 4-Carboxymethyl piperazine [PPac], 1,13-diamino-4,7,10-trioxatridecan-succinamic acid [Ttds], 8-amino-3,6-dioxaoctanoic acid [O2Oc], 2-(4-(amino)piperidin-1-yl)acetic acid [APac], 4-trans-aminomethylcyclohexane carboxylic acid [4Amc] μ-Alanine [Bal], γ-Aminobutyric acid [Gab], 5-amino pentanoic acid [Ava], 6-aminohexanoic acid [Ahx], 3-aminomethyl-benzoic acid [Mamb], 4-aminomethyl-benzoic acid [Pamb] and an ε-amino acid of formula (35):

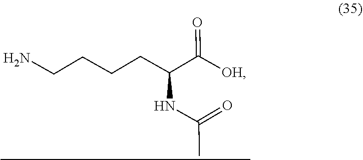

11-17. (canceled)

18. The compound of claim 1, wherein Xaa2 is a residue of an L-α-amino acid selected from the group consisting of a polar amino acid, an aromatic amino acid, and a charged amino acid, wherein Xaa2 is preferably a residue of an L-α-amino acid selected from the group consisting of Gln, Tyr, (S)-N-methyl-tyrosine [Nmy], Phe, Arg, (S)-dimethylornithine [Dmo], Ser, Thr, Asp, Glu and 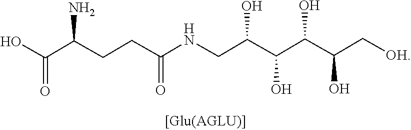 [SMILES: N[C@@H](CCC(=O)NC[C@H](O)[C@@H](O)[C@H](O)[C@H](O)CO)C(=O)O]
wherein Xaa2 is more preferably a residue of an L-α-amino acid selected from the group consisting of Gln, Tyr, (S)-N-methyl-tyrosine [Nmy], Arg, (S)-dimethylornithine [Dmo] and Ser, and wherein Xaa2 is most preferably a residue of Gln.

19-22. (canceled)

23. The compound of claim 1, wherein Xaa2 is a residue of an L-α-amino acid comprising, in addition to an amino group and a carboxy group attached to an α-C atom, a functional group FG1 forming a covalent linkage B1 with a functional group FG2 of Xaa11, wherein Xaa11 is a residue of an L-α-amino acid comprising, in addition to an amino group and a carboxy group attached to an α-C atom, the functional group FG2, such that a bicyclic peptide of formula (1b) is formed: 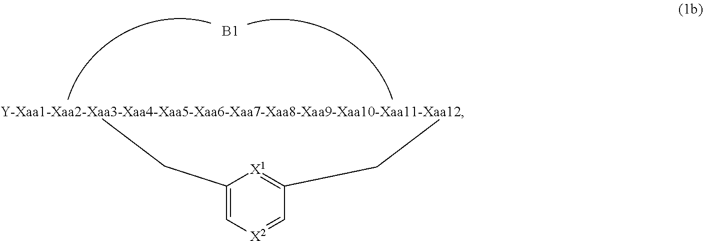
wherein the covalent linkage B1 is preferably selected from the group consisting of an amide linkage, a disulfide linkage, a thioether linkage, a thiourea linkage, a triazole linkage, a carbamate linkage, an amine linkage, a sulfonamide linkage, an ester linkage, a thioester linkage, an ether linkage, a urea linkage and a hydrocarbon linkage,
wherein the covalent linkage B1 is more preferably selected from the group consisting of an amide linkage or a disulfide linkage, and
wherein the functional group FG1 of Xaa2 forming the covalent linkage B1 with the functional group FG2 of Xaa11 is preferably selected from the group consisting of NH₂, NH—, COOH, activated carboxylic acid, chloro, bromo, iodo, SH, OH, SOOH, activated sulfonic acid, sulfonic acid ester, Michael acceptors, isocyanate, isothiocyanate, azide, alkene, and alkyne, and/or
wherein the functional group FG2 of Xaa11 forming the covalent linkage B1 with the functional group FG1 of Xaa2 is selected from the group consisting of NH₂, NH—, COOH, activated carboxylic acid, chloro, bromo, iodo, SH, OH, SOOH, activated sulfonic acid, sulfonic acid ester, Michael acceptors, isocyanate, isothiocyanate, azide, alkene and alkyne;
wherein Xaa2 is preferably a residue of an L-α-amino acid selected from the group consisting of (S)-2,3-diaminopropionic acid [Dap], (S)-2,4-diaminobutyric acid [Dab], (S)-ornithine [Orn], Lys, Cys, (S)-homocysteine [Hcy], (R)-Penicillamine [Pen], Asp and Glu, wherein Xaa2 is more preferably a residue of Glu.

24-29. (canceled)

30. The compound of claim 23, wherein Xaa11 is a residue of an L-α-amino acid selected from the group consisting of (S)-2,3-diaminopropionic acid [Dap], (S)-2,4-diaminobutyric acid [Dab], (S)-ornithine [Orn], Lys, Cys, (S)-homocysteine [Hcy], (R)-Penicillamine [Pen], Asp and Glu, wherein Xaa11 is preferably a residue of (S)-2,3-diaminopropionic acid [Dap].

31. (canceled)

32. (canceled)

33. The compound of claim 1, wherein Xaa4 is a residue of an L-α-amino acid selected from the group consisting of a charged amino acid, an aliphatic amino acid, and a polar amino acid and, wherein Xaa4 is preferably a residue of an L-α-amino acid selected from the group consisting of Glu, Ala, Ser, (S)-homoserine [Hse], (S)-N-methyl-serine [Nms], Gln, Asn, Asp, Dmo and 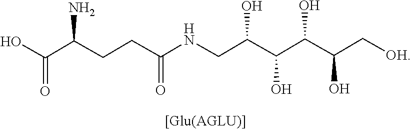 [SMILES: N[C@@H](CCC(=O)NC[C@H](O)[C@@H](O)[C@H](O)[C@H](O)CO)C(=O)O]
wherein Xaa4 is more preferably a residue of an L-α-amino acid selected from the group consisting of Glu, Ala, Ser, Gln and (S)-homoserine [Hse], and wherein Xaa4 is most preferably a residue of Glu.

34-36. (canceled)

37. The compound of claim 1, wherein Z3 is absent from Xaa5, wherein Xaa5 is preferably a residue of an amino acid selected from the group consisting of D-pro, Gly, N-methyl-glycine [Nmg], D-ala, (R)-piperidine-2-carboxylic acid [D-pip], (R)-azetidine-2-carboxylic acid [D-aze], (R)-N-methyl-alanine [Nma], and 2-amino-isobutyric acid [Aib], and wherein Xaa5 is more preferably a residue of D-pro.

38. (canceled)

39. (canceled)

40. The compound of claim 1, wherein Xaa5 is a residue of an amino acid bound to Z3, wherein Z3 comprises an effector E3, such as a chelator, and a linker moiety L3-, wherein Xaa5 is preferably a residue of an amino acid selected from the group consisting of N—(C₁-C₄)alkyl glycine, a non-aromatic D-α-amino acid, a non-aromatic N-Methyl-D-α-amino acid, a cyclic D-α-amino acid, and an α,α-dialkylamino acid, which comprises at least one functional group forming a covalent linkage with the linker moiety L3.

41. (canceled)

42. The compound of claim 40, wherein Z3 is an effector E3, wherein Xaa5 is preferably a residue of an amino acid selected from the group consisting of 4-aminobutyl-glycine [Nlys], D-lys, (R)-ornithine [D-orn], (R)-2,4-diaminobutyric acid [D-dab], and (R)-2,3-diaminopropionic acid [D-dap], and the effector E3 is covalently attached to an N atom different from the α-nitrogen atom of any one of Nlys, D-lys, D-orn, D-dab, and D-dap, and wherein the bond linking the effector E3 to the N atom different from the α-nitrogen atom is preferably an amide bond.

43. (canceled)

44. (canceled)

45. The compound of claim 40, wherein Z3 comprises an effector E3 and a linker moiety L3-, wherein Xaa5 is preferably a residue of an amino acid selected from the group consisting of 4-aminobutyl-glycine [Nlys], D-lys, (R)-ornithine [D-orn], (R)-2,4-diaminobutyric acid [D-dab], and (R)-2,3-diaminopropionic acid [D-dap], and the chelator is covalently attached to an N atom different from the α-nitrogen atom of any one of Nlys, D-lys, D-orn, D-dab, and D-dap, and/or,
wherein the linker moiety L3 preferably provides (a) a carboxy group forming an amide bond with the N atom different from the α-nitrogen atom of any one of 4-aminobutyl-glycine [Nlys], D-lys, (R)-ornithine [D-orn], (R)-2,4-diaminobutyric acid [D-dab], and (R)-2,3-diaminopropionic acid [D-dap], and (b) an amino group forming a covalent bond to the effector E3,
wherein the linker moiety L3 is preferably selected from the group consisting of X31 and X31-X32, wherein X31 and X32 are each and individually a residue of an amino acid, wherein if the linker moiety L3 is X31, a carboxy group is provided by X31 and if the linker moiety L3 is X31-X32, a carboxy group is provided by X32, wherein the carboxy group of L3 forms an amide bond with an N atom different from the α-nitrogen atom of any one of 4-aminobutyl-glycine [Nlys], D-lys, (R)-ornithine [D-orn], (R)-2,4-diaminobutyric acid [D-dab], and (R)-2,3-diaminopropionic acid [D-dap], and X3 provides an amino group which is forming a covalent bond to the effector E3,
wherein X31 and X32 are preferably each and individually a residue of an amino acid selected from the group consisting of 1,13-diamino-4,7,10-trioxatridecan-succinamic acid [Ttds], 8-amino-3,6-dioxaoctanoic acid [O2Oc], 2-(4-(amino)piperidin-1-yl)acetic acid [APac], 4-Carboxymethyl piperazine [PPac], 4-trans-aminomethylcyclohexane carboxylic acid [4Amc] and an amino acid according to any one of formulae (32)-(34); 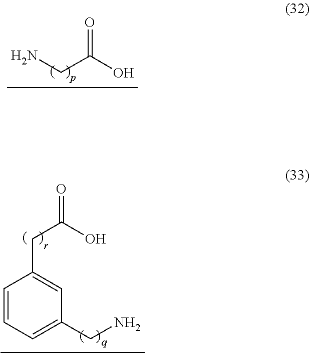
and the ortho-para-substituted isomers thereof, and 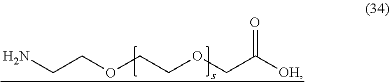
wherein
p is 2, 3, 4, 5, 6, 7, 8, 9, or 10,
q is 0, 1, 2, 3, or 4,
r is 0, 1, 2, 3, or 4,
s is 0, 1, 2, 3, 4, 5, 6, 7, 8, 9, 10, 11 or 12,
and the amino acid of formulae (32) and (33) is optionally substituted, and
wherein the amino acid of formulae (32) and (33) is preferably substituted with R^{X11}—CO—NH— at an α-carbon atom which is covalently bound to the COOH-group in formulae (32) and (33), wherein R^{X11} is selected from the group consisting of (C₁-C₁₀)alkyl, (C₅-C₁₀)aryl, and (C₁-C₅)alkyl-(C₅-C₁₀)aryl, R^{X11} being preferably methyl,
wherein X31 and X32 are preferably each and individually a residue of an amino acid selected from the group consisting of 1,13-diamino-4,7,10-trioxatridecan-succinamic acid [Ttds], 8-amino-3,6-dioxaoctanoic acid [O2Oc], 2-(4-(amino)piperidin-1-yl)acetic acid [APac], 4-Carboxymethyl piperazine [PPac], 4-trans-aminomethylcyclohexane carboxylic acid [4Amc] β-Alanine [Bal], γ-Aminobutyric acid [Gab], 5-amino pentanoic acid [Ava], 6-aminohexanoic acid [Ahx], 3-aminomethyl-benzoic acid [Mamb], 4-aminomethyl-benzoic acid [Pamb] and an ε-amino acid of formula (35):

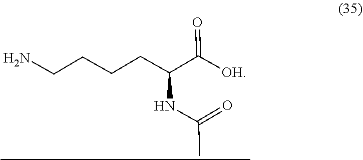

46-52. (canceled)

53. The compound of claim 40, wherein the effector E3 is selected from the group consisting of:
(α) a moiety derived from a chromophore, wherein the chromophore is preferably selected from (α1) a phosphorophore and (α2) a fluorophore such as fluorescein or rhodamine; and
(β) a chelator optionally comprising a chelated nuclide; and
(γ) a moiety derived from a drug, preferably from a cytotoxic drug.

54. The compound of claim 1, wherein Xaa6 is a residue selected from:
a residue of a polar N-methylated L-α-amino acid, a residue of a neutral α-amino acid, and wherein the neutral α-amino acid is preferably Ala,
a residue of an S-alkylated cysteine, and a residue of a sulfoxide or sulfone of an S-alkylated cysteine.

55-58. (canceled)

59. The compound of claim 1, wherein Xaa6 is a residue of an amino acid according to formula (3) and R^{6a} is selected from the group consisting of (C₁-C₁₀)alkyl, (C₅-C₁₀)aryl, (C₁-C₅)alkyl-(C₅-C₁₀)aryl and (C₃-C₇)cycloalkyl-(C₅-C₁₀)aryl, wherein R^{6c} is preferably (C₁-C₄)alkyl.

60. (canceled)

61. The compound of claim 1, wherein Xaa6 is a residue of an amino acid which is selected from the group consisting of Asp Ala, Asn, (S)-homoserine [Hse], Gln, Glu, Lys, (S)-ornithine [Orn], (S)-2,4-diaminobutyric acid [Dab], N-Methyl-Asp, (S)-benzylcysteine [C(Bzl)], (S)-2-amino-3-(quinolin-2-ylmethylsulfanyl)-propionic acid [C(2Quyl)], (S)-benzyl-cysteine-sulfone [Eem], (S)-4-benzyloxy-L-phenylalanine [Tyr(Bzl)], and (S)-2-amino-4-[(naphthalen-1-ylmethyl)-carbamoyl]-butyric acid [E(NHMe2Nph)], wherein Xaa6 is preferably a residue of Asp.

62. (canceled)

63. The compound of claim 1, wherein Xaa6 is a residue of an L-α-amino acid comprising, in addition to an amino group and a carboxy group attached to an α-C atom, a functional group FG3 forming a covalent linkage B2 with a functional group FG4 of Xaa11, wherein Xaa11 is a residue of an α-amino acid comprising, in addition to an amino group and a carboxy group attached to an α-C atom, the functional group FG4, wherein a bicyclic peptide of formula (1c) is formed 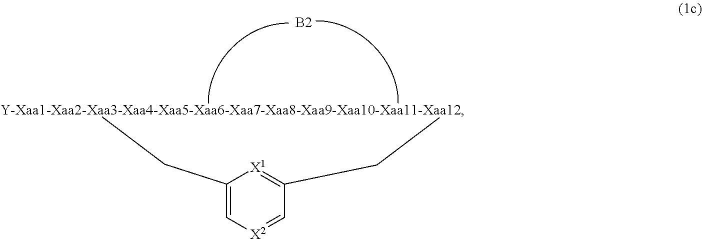
wherein the covalent linkage B2 is preferably selected from the group consisting of an amide linkage, a disulfide linkage, a thioether linkage, a thiourea linkage, a triazole linkage, a carbamate linkage, an amine linkage, a sulfonamide linkage, an ester linkage, a thioester linkage, an ether linkage, a urea linkage and a hydrocarbon linkage, wherein the covalent linkage B2 is more preferably selected from the group consisting of an amide linkage or a disulfide linkage, and
wherein the functional group FG3 of Xaa6 forming the covalent linkage B2 with the functional group FG4 of Xaa11 is preferably selected from the group consisting of NH₂, NH—, COOH, activated carboxylic acid, chloro, bromo, iodo, SH, OH, SOOH, activated sulfonic acid, sulfonic acid ester, Michael acceptors, isocyanate, isothiocyanate, azide, alkene, and alkyne, and/or
wherein the functional group FG4 of Xaa11 forming the covalent linkage B2 with a functional group FG3 of Xaa6 is selected from the group consisting of NH₂, NH—, COOH, activated carboxylic acid, chloro, bromo, iodo, SH, OH, SOOH, activated sulfonic acid, sulfonic acid ester, Michael acceptors, isocyanate, isothiocyanate, azide, alkene and alkyne,
wherein Xaa6 is preferably a residue of an L-α-amino acid selected from the group consisting of (S)-2,3-diaminopropionic acid [Dap], (S)-2,4-diaminobutyric acid [Dab], (S)-ornithine [Orn], Lys, Cys, (S)-homocysteine [Hcy], (R)-penicillamine [Pen], Asp and Glu, and/or wherein Xaa11 is a residue of an L-α-amino acid selected from the group consisting of (S)-2,3-diaminopropionic acid [Dap], (S)-2,4-diaminobutyric acid [Dab], (S)-ornithine [Orn], Lys, Cys, (S)-homocysteine [Hcy], (R)-penicillamine [Pen] Asp, D-asp, D-glu and Glu.

64-69. (canceled)

70. The compound of claim 1, wherein Xaa7 is a residue of an aromatic amino acid which may be substituted at the aromatic ring system with at least one substituent.

71. (canceled)

72. The compound of claim 1, wherein Xaa7 is a residue of an amino acid selected from the group consisting of a modified 3-aminophenyl alanine [Af3(R^{7c})] of formula (4a): 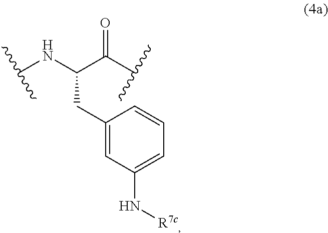
a modified 4-aminophenyl alanine [Aph(R^{7d})] of formula (4b): 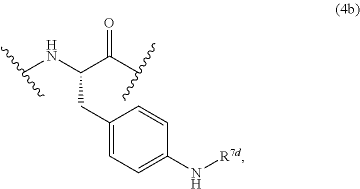
a substituted (S)-3-benzothienyl alanine [Bta], a substituted Trp, and a substituted Phe,
wherein
the substituted Bta and the substituted Trp are each and individually substituted at the aromatic ring with a substituent selected from the group consisting of a halogen, methyl, and OH,
wherein in the substituted Bta and the substituted Trp, one or two of the aromatic carbon atoms may be replaced by an N-atom,
the substituted Phe is substituted at the aromatic ring with one, two or three substituents, wherein each and any of the substituents is individually and independently selected from the group consisting of a halogen, methyl, OH, NH₂, O—R^{7a}, wherein
R^{7a} is (C₁-C₆)alkyl, and
wherein, in formula (4a),
R^{7c} is —CO—R^{7e},
wherein
R^{7e} is selected from the group consisting of (C₁-C₅)alkyl, (C₅-C₁₀)aryl and (C₅-C₁₀)heterocyclyl, wherein
(C₁-C₅)alkyl is optionally substituted with a substituent selected from the group consisting of OH, SO₂NH₂, SO₂NH—R^{7f}, CO(NHOH), COOH, CONH₂ and NH₂,
one alkyl carbon atom of (C₁-C₅)alkyl is optionally replaced by an atom or moiety each selected from the group consisting of an ether oxygen and a sulfone (SO₂) moiety,
(C₅-C₁₀)aryl is optionally substituted with a substituent selected from the group consisting of a halogen, OH, SO₂NH₂, SO₂NH—R^{7f}, CO(NHOH), COOH, CONH₂ and NH₂, and
(C₅-C₁₀)heterocyclyl is optionally substituted with a substituent selected from the group consisting of a halogen, OH, SO₂NH₂, SO₂NH—R^{7f}, NH—SO—NH₂, CO(NHOH), COOH, CONH₂ and NH₂,
wherein
R^{7f} is (C₁-C₄)alkyl,
wherein, in formula (4b),
R^{7d} is —CO—R^{7g},
wherein
R^{7g} is (C₁-C₅)alkyl, (C₅-C₁₀)aryl and (C₅-C₁₀)heterocyclyl,
wherein
(C₁-C₅)alkyl is optionally substituted with a substituent selected from the group consisting of OH, SO₂NH₂, SO₂NH—R^{7h}, CO(NHOH), COOH, CONH₂ and NH₂,
one alkyl carbon atom of (C₂-C₅)alkyl is optionally replaced by an atom or moiety each selected from the group consisting of an ether oxygen and a sulfone (SO₂) moiety,
(C₅-C₁₀)aryl is optionally substituted with a substituent selected form the group consisting of a halogen, OH, SO₂NH₂ SO₂NH—R^{7h}, CO(NHOH), COOH, CONH₂ and NH₂, and
(C₅-C₁₀)heterocyclyl is optionally substituted with a substituent selected from the group consisting of a halogen, OH, SO₂NH₂, SO₂NH—R^{7h}, NH—SO—NH₂, CO(NHOH), COOH, CONH₂ and NH₂, and
wherein R^{7h} is (C₁-C₄)alkyl.

73. The compound of claim 72, wherein Xaa7 is a residue of an amino acid, wherein the amino acid is selected from the group consisting of:
modified 3-aminophenyl alanine [Af3(R^{7c})] of formula (4a): 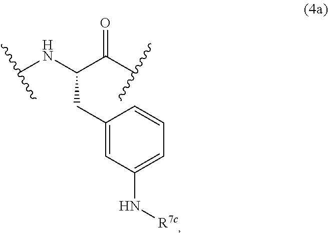
modified 4-aminophenyl alanine [Aph(R^{7d})] of formula (4b): 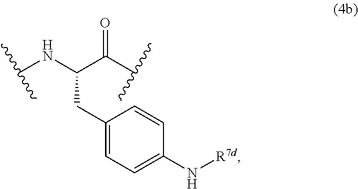
substituted Trp, substituted (S)-3-benzothienyl alanine [Bta], (S)-3-(1-naphthyl)alanine [1Ni], (S)-4-benzyloxy-L-phenylalanine [Tyr(Bzl)], Tyr, substituted Phe and (S)-benzylcysteine [Cys(Bzl)], preferably Xaa7 is a residue of modified 3-aminophenyl alanine [Af3(R^{7c})] of formula (4a) or of modified 4-aminophenyl alanine [Aph(R^{7d})] of formula (4b),
wherein Xaa7 is preferably a residue of an amino acid selected from the group consisting of: D/L-1-methyltryptophane [1MW], D/L-7-methyltryptophane [7MW], 5-chloro-tryptophane [5Clw], DL-5-methyl-tryptophane [Egc], substituted [Bta], (S)-4-benzyloxy-L-phenylalanine [Tyr(Bzl)], (S)-3-(1-naphthyl)alanine [1Ni], (2S)-2-amino-3-[3-(trifluoromethyl)phenyl]propanoic acid [Mtf], (2S)-2-amino-3-[4-(trifluoromethyl)phenyl]propanoic acid [Ptf], (S)-3,4-dichlorophenylalanine [Eaa], 4-(tert-butyl)-phenylalanine [Eap], (2S)-2-amino-3-(4-iodophenyl)propanoic acid [Pif], (S)-biphenylalanine [Bip], (S)-3,3-diphenylalanine [Dip], (S)-benzylcysteine [Cys(Bzl)], the modified 3-aminophenyl alanine [Af3(R^{7c})] of formula (4a) and modified 4-aminophenyl alanine [Aph(R^{7d})] of formula (4b),
wherein R^{7c} is selected from the group consisting of: 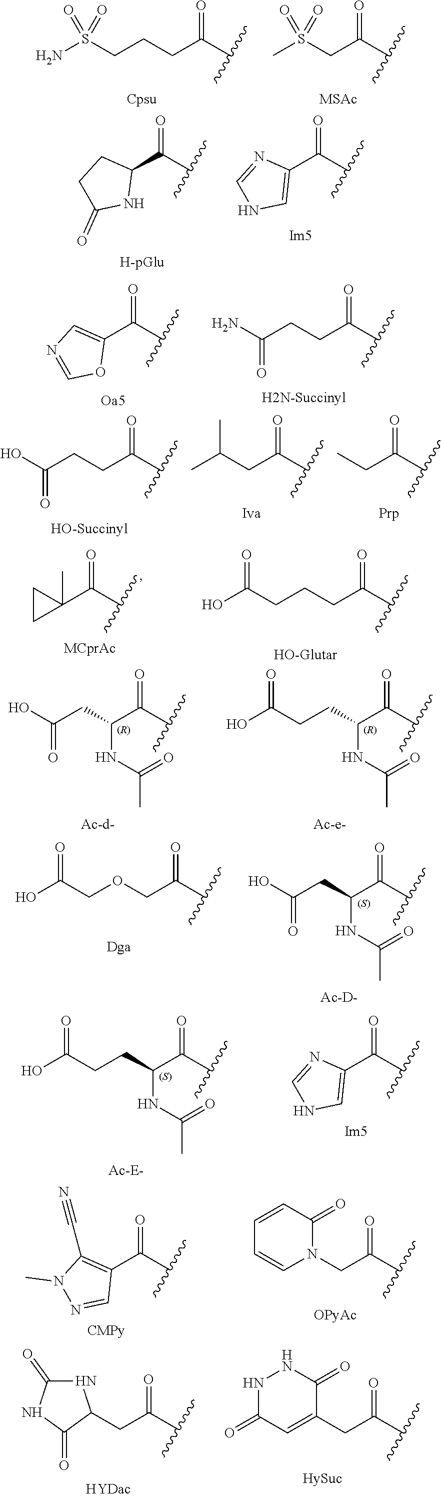 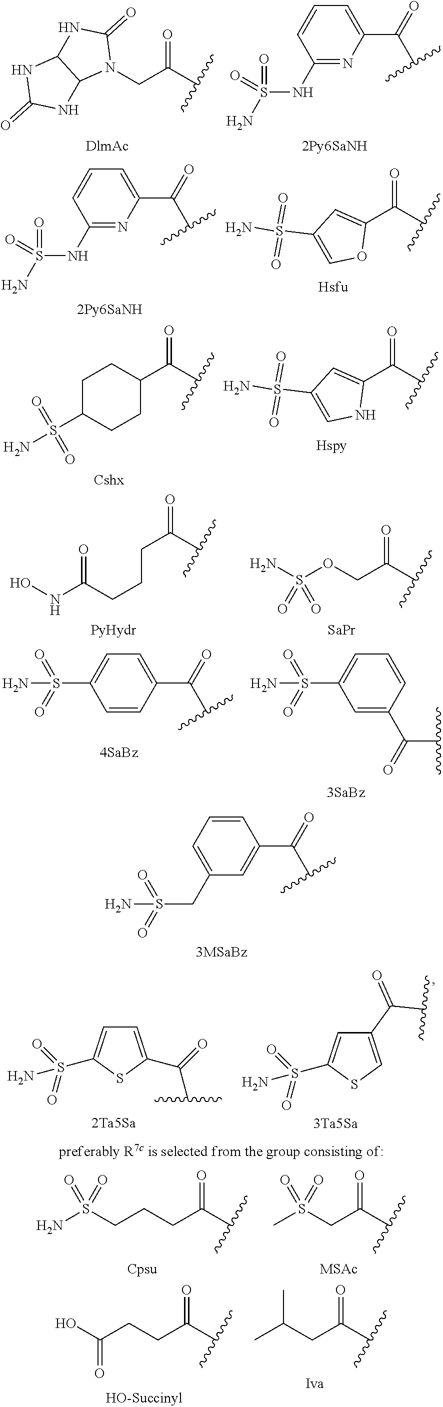 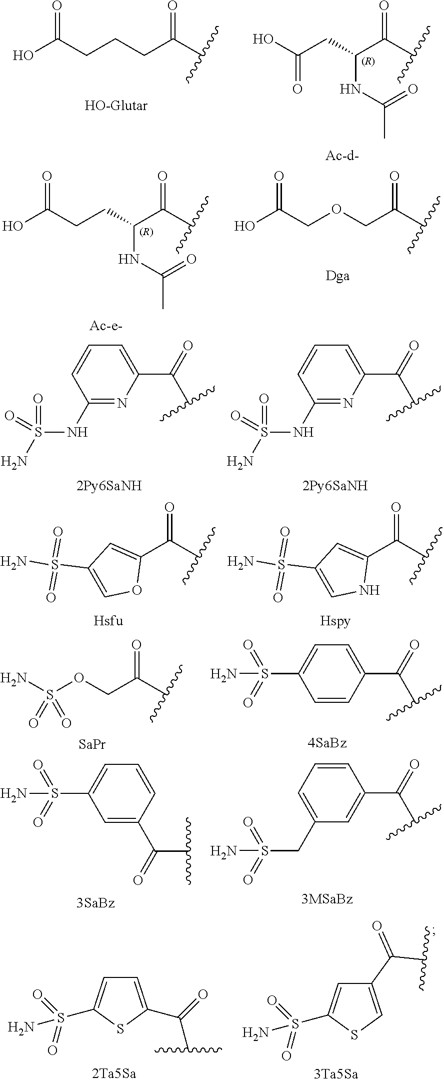
wherein R^{7d} is selected from the group consisting of: 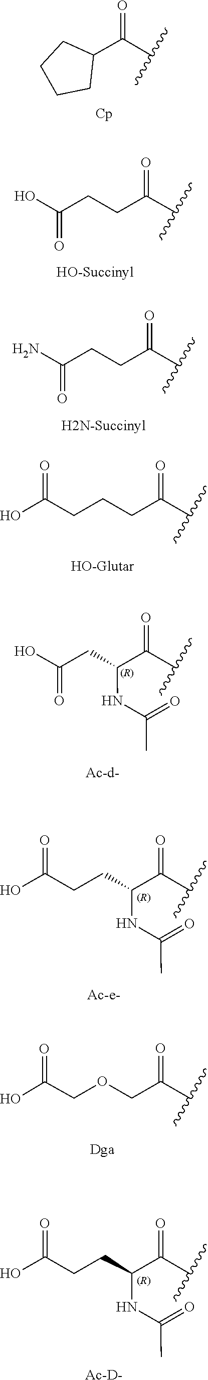 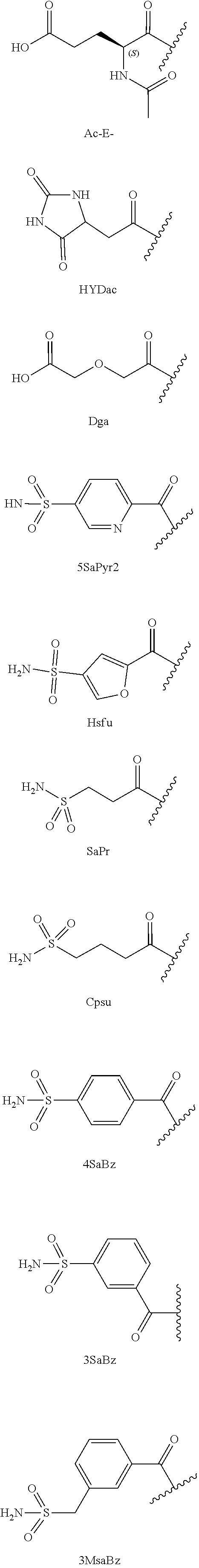 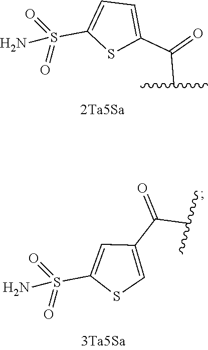 [SMILES: *C(=O)c1ccc(S(N)(=O)=O)s1 ; *C(=O)c1csc(S(N)(=O)=O)c1]
preferably R^{7d} is selected from the group consisting of: 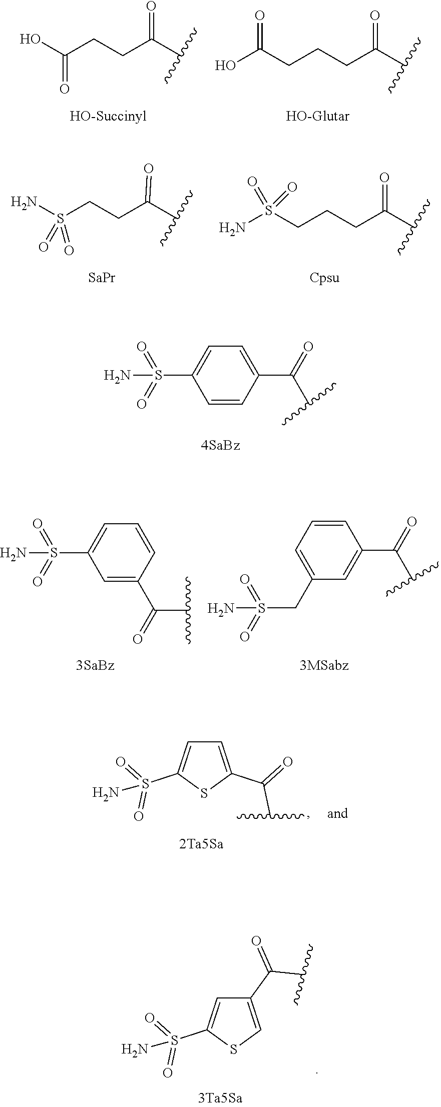
wherein Xaa7 is more preferably a residue of an amino acid selected from the group consisting of the modified 3-aminophenyl alanine [Af3(R^{7c})] of formula (4a) and the modified 4-aminophenyl alanine [Aph(R^{7d})] of formula (4b), wherein
R^{7c} is selected from the group consisting of: 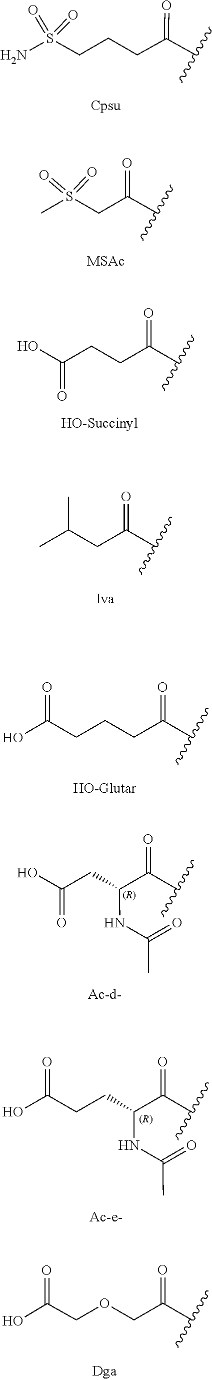 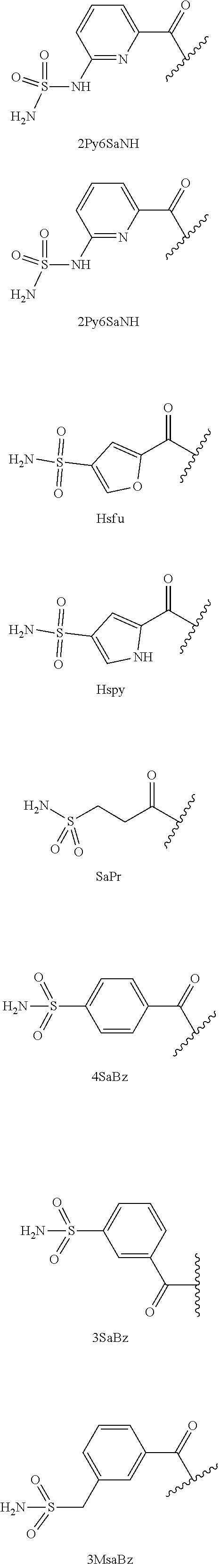 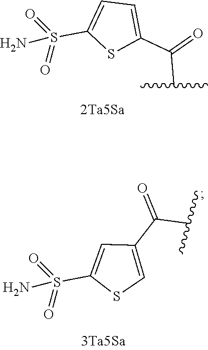 [SMILES: *C(=O)c1ccc(S(N)(=O)=O)s1 ; *C(=O)c1csc(S(N)(=O)=O)c1]
and wherein R^{7d} is selected from the group consisting of:

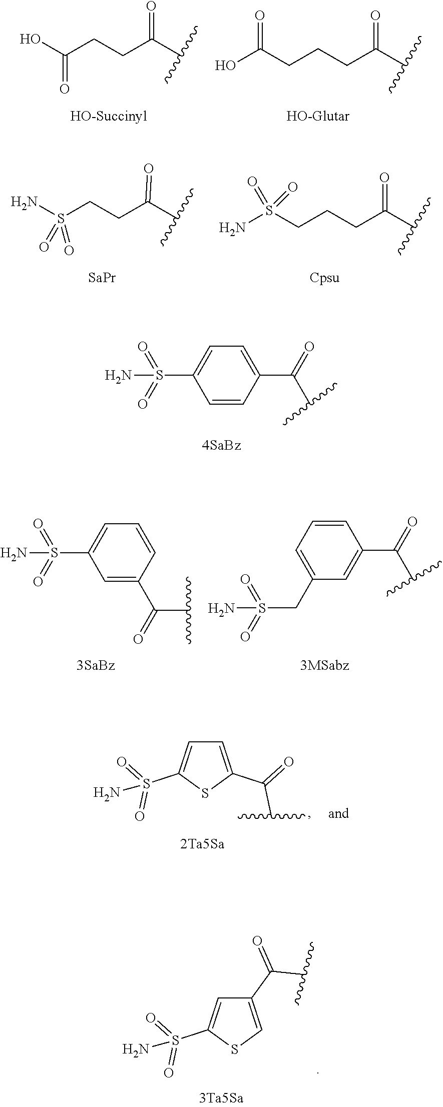

74. (canceled)

75. (canceled)

76. The compound of claim 73,
wherein Xaa7 is:
a residue of the modified 3-aminophenyl alanine [Af3(R^{7c})] of formula (4a), wherein R^{7c} is 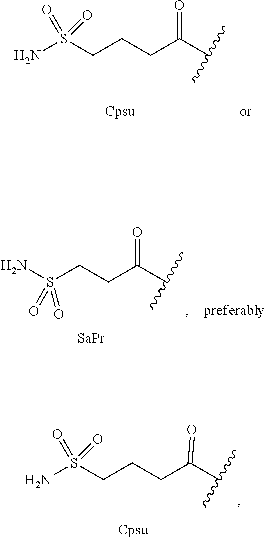 [SMILES: *C(=O)CCCS(N)(=O)=O ; *C(=O)CCS(N)(=O)=O ; *C(=O)CCCS(N)(=O)=O]
a residue of the modified 4-aminophenyl alanine [Aph(R^{7d})] of formula (4b), wherein R^{7d} is 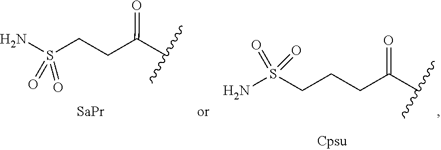 [SMILES: *C(=O)CCS(N)(=O)=O ; *C(=O)CCCS(N)(=O)=O]
preferably SaPr

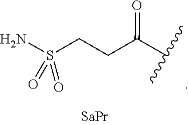 [SMILES: *C(=O)CCS(N)(=O)=O]

77. (canceled)

78. The compound of claim 70, wherein Xaa7 is a residue of an aromatic amino acid selected from the group consisting of (S)-3-benzothienyl alanine [Bta], Trp and Phe.

79. The compound of claim 1, wherein Xaa8 is a residue of an aliphatic L-α-amino acid of formula (IX) or an amino acid of formula (XI): 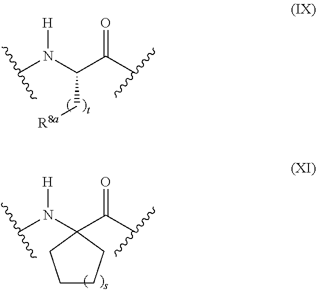
wherein
R^{8a} is selected from the group consisting of (C₁-C₄)alkyl, (C₃-C₇)cycloalkyl and H,
t=0, 1, 2, 3, or 4
s=0, 1, 2 or 3
wherein
in the amino acid of formula (XI) one aryl-ring is optionally annulated to a ring bond which does not include the α-C-atom, and
in the carbocyclic part of the amino acid of formula (XI) a CH₂ group which is spaced at least one carbon atom apart from the α-carbon atom is optionally replaced by an O atom or a NH group,
wherein Xaa8 is preferably a residue of an amino acid selected from the group consisting of Leu, Nle, Npg, Cha, Aic, Thp, Eca, and Egz, and,
wherein Xaa8 is more preferably a residue of Leu.

80. (canceled)

81. (canceled)

82. The compound of claim 1, wherein Xaa9 is a residue of an amino acid selected from the group consisting of an L-α-amino acid of formula (XIII) and Gly: 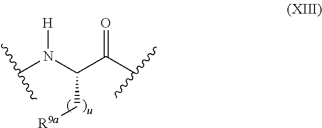
wherein
R^{9a} is selected from the group consisting of X⁹, H, OH, COOH, CONH₂, N(R^{9b})₂, CONH—R^{9c} and —NH—CO—X⁹,
wherein
X⁹ is selected from the group consisting of (C₁-C₆)alkyl, (C₅-C₁₀)aryl and (C₃-C₁₀)heteroaryl, and X⁹ is substituted with one or two substituents each and individually selected from the group consisting of OH methyl, CONH₂, a halogen, and NH₂;
u=1, 2, 3 or 4, wherein optionally one or two hydrogens of the 3-CH₂ group and/or of the γ-CH₂-group are each and individually substituted by methyl and/or one of the hydrogens of the β-CH₂-group is optionally substituted by OH,
R^{9b} is each and independently selected from the group consisting of (C₁-C₄)alkyl and H,
R^{9c} is selected from the group consisting of (C₁-C₈)alkyl, and (C₁-C₈)cycloalkyl optionally substituted with 1, 2, 3, 4, 5, or 6 OH-groups under the proviso and that each carbon atom is bound to no or one O or N-atom-,
wherein Xaa9 is preferably a residue of an amino acid selected from the group consisting of Thr, Gly, Ala, His, (S)-dimethylornithine [Dmo], and 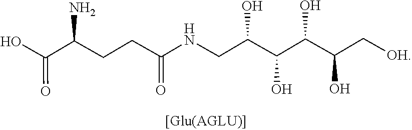 [SMILES: N[C@@H](CCC(=O)NC[C@H](O)[C@@H](O)[C@H](O)[C@H](O)CO)C(=O)O]
wherein Xaa9 is more preferably a residue of Thr.

83. (canceled)

84. (canceled)

85. The compound of claim 1, wherein Xaa10 is selected from the group consisting of Trp optionally substituted with a substituent selected from the group consisting of methyl, a halogen or OH, and an aza-analogue of Trp optionally substituted with methyl, a halogen or OH, and wherein Xaa10 is preferably a residue of an amino acid selected from the group consisting of Trp and (S)-7-aza-tryptophane [7Nw].

86. (canceled)

87. The compound of claim 1, wherein Xaa11 is a residue of an amino acid which is selected from the group consisting of an L-α-amino acid and Gly and Z4 is absent, wherein Xaa11 is preferably a residue of an L-α-amino acid and the L-α-amino acid is Ser.

88. (canceled)

89. The compound of claim 1,
wherein Xaa11 is:
a residue of an L-α-amino acid comprising, in addition to an amino group and a carboxy group attached to an α-C atom, the functional group FG2, and Xaa2 is a residue of an L-α-amino acid comprising, in addition to an amino group and a carboxy group attached to an α-C atom, the functional group FG1 forming the covalent linkage B1 with the functional group FG2 of Xaa11, such that the bicyclic peptide of formula (1b) is formed: 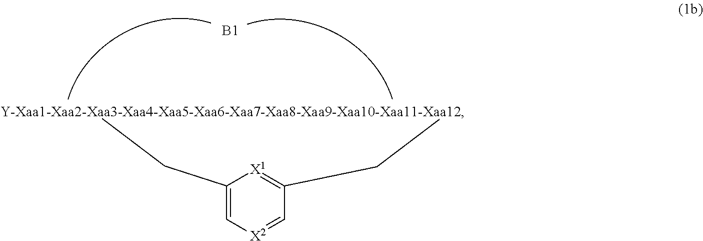
is a residue of an L-α-amino acid comprising, in addition to an amino group and a carboxy group attached to an α-C atom, the functional group FG4, and Xaa6 is a reside of an L-α-amino acid comprising, in addition to an amino group and a carboxy group attached to an α-C atom, the functional group FG3 forming the covalent linkage B2 with the functional group FG4 of Xaa11, wherein the bicyclic peptide of formula (1c) is formed:

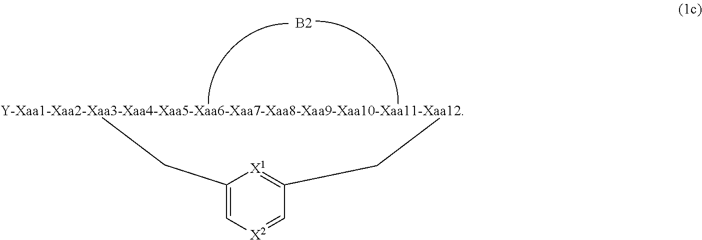

90. (canceled)

91. The compound of claim 1, wherein Xaa11 is a residue of an amino acid which is selected from the group consisting of Gly and an L-α-amino acid, wherein the L-α-amino acid is bound to Z4, wherein Z4 comprises an effector E4 and a linker moiety L4, wherein Xaa11 is preferably a residue of an L-α-amino acid selected from the group consisting of Glu, Gln, and an L-α-amino acid of formula (XI): 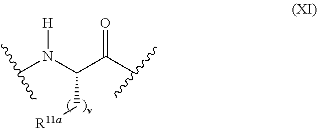
wherein
v=1, 2, 3 or 4,
R^{11a} is selected from the group consisting of H, OH, COOH, CONH₂, NH—(C═NH)—NH₂, N(R^{11b})₂, CONH—R^{11c}, —CO(Z4), X¹³ and —NH—CO—X¹³, NH—CO(Z4), O—CO(Z4), Z4 and NH—CS—Z4, wherein
X¹³ is selected from the group consisting of (C₁-C₆)alkyl, (C₅-C₆)aryl and (C₃-C₅)heteroaryl and X¹³ is optionally substituted with one or two substituents each and individually selected from the group consisting of methyl, CONH₂, a halogen, NH₂ and OH,
R^{11b} is each and independently selected from the group consisting of (C₁-C₄)alkyl and H, and
R^{11c} is selected from the group consisting of (C₁-C₈)alkyl, and (C₁-C₈)cycloalkyl optionally substituted by 1, 2, 3, 4, 5, or 6 OH-groups under the proviso that each carbon atom is bound to no or one O or N-atom,
optionally one or two hydrogens of the β-CH₂ group and/or of the γ-CH₂-group in formula (XI) are each and individually substituted by methyl, and
one of the hydrogens of the β-CH₂-group in formula (XI) is optionally substituted by OH,
wherein Xaa11 is more preferably a residue of an amino acid selected from the group consisting of Ala, Ser, Gly, Arg, Lys, (S)-dimethylornithine [Dmo], and 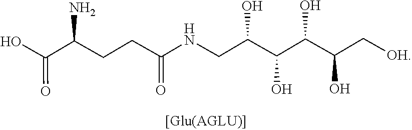 [SMILES: N[C@@H](CCC(=O)NC[C@H](O)[C@@H](O)[C@H](O)[C@H](O)CO)C(=O)O]
and
wherein Xaa11 is most preferably a residue of Ser.

92-94. (canceled)

95. The compound of claim 91, wherein the linker moiety L4 covalently links the chelator to the L-α-amino acid of Xaa11, wherein the L-α-amino acid Xaa11 preferably includes a functional group FG5 different from the carboxyl group and the amino group attached to the α-C atom of Xaa11, and the linker moiety L4 covalently links the effector E4 to the functional group FG5 of the L-α-amino acid of Xaa11, wherein Xaa11 is more preferably a residue of an L-α-amino acid of formula (XI) and the functional group FG5 is provided by R^{11a}, and
wherein the linker moiety L4 preferably provides (a) a first amino group forming a covalent bond with the functional group FG5 of the L-α-amino acid of Xaa11 and (b) a second amino group forming a covalent bond to the effector E4.

96-98. (canceled)

99. The compound of claim 91, wherein the linker moiety L4 is either X41 or a residue selected from the group consisting of X41-X42 and X42-X41, wherein
X41 is a residue of a diamine providing a first amino group and a second amino group,
X42 is a residue of an amino acid providing an amino group and a carboxy group,
X41-X42 is a residue of a diamine, wherein the diamine provides a first amino group and a second amino group,
wherein the first amino group is the first amino group of X41,
the second amino group is the amino group of X42, and
the second amino group of X41 forms an amide bond with the carboxy group of X42, and
X42-X41 is a residue of a diamine, wherein the diamine provides a first amino group and a second amino group,
wherein the first amino group is the amino group of X42,
the second amino group is the second amino group of X41, and
the carboxy group of X42 forms an amide bond with the first amino group of X41,
wherein X41 is preferably a residue of a linear or a cyclic diamine.

100. (canceled)

101. The compound of claim 91, wherein Xaa11 is a residue of an L-α-amino acid of formula (XI) and R^{11a} is selected from the group consisting of —CO(Z4), —NH—CO(Z4), —O—CO(Z4), —Z4 and —NH—CS—Z4, wherein R^{11a} is preferably —CO(Z4) and L4 is covalently attached to the carbonyl carbon atom comprised in R^{11a} by means of an amide bond.

102. (canceled)

103. The compound of claim 99, wherein X41 is a residue of a diamine which is selected from the group consisting of a diamine of any one of formulae (35) to (37) 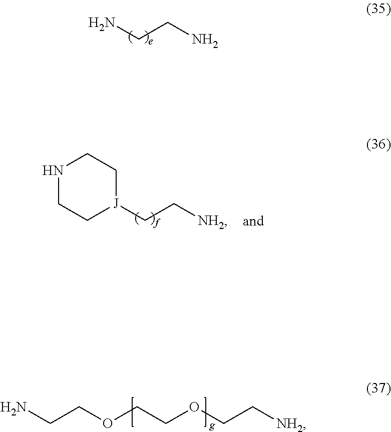
wherein
e is 1, 2, 3, 4, 5, 6, 7, 8, 9, or 10,
f is 0, 1, 2, 3, 4, 5 or 6,
g is 0, 1, 2, 3, 4, 5, 6, 7, 8, 9, 10, 11, or 12,
the diamine of any one of formulae (35) and (36) is optionally substituted with —CONH₂, and
J is selected from the group consisting of CH and N,
wherein, in the diamine of any one of formulae (35) and (36), the carbon atom which is substituted with a nitrogen atom is preferably further substituted with —CONH₂.

104. (canceled)

105. The compound of claim 99, wherein X41 is a residue of a diamine selected from the group consisting of 1,3-diaminopropane [Apr], 1,5-diaminopentane [Ape], diaminobutane and ethylendiamine, and/or
wherein X42 is a residue of an amino acid selected from the group consisting of 1,13-diamino-4,7,10-trioxatridecan-succinamic acid [Ttds], 8-amino-3,6-dioxaoctanoic acid [O2Oc], 2-(4-(amino)piperidin-1-yl)acetic acid [APac], 4-carboxymethyl piperazine [PPac], 4-trans-aminomethylcyclohexane carboxylic acid [4Amc] and an amino acid of any one of formulae (32), (33) and (34): 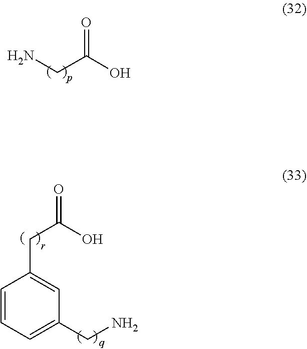
and the ortho- and para-substituted isomers thereof, and 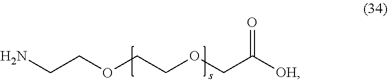
wherein
p is 2, 3, 4, 5, 6, 7, 8, 9, or 10,
g is 0, 1, 2, 3, or 4,
r is 0, 1, 2, 3, or 4,
s is 0, 1, 2, 3, 4, 5, 6, 7, 8, 9, 10, 11 or 12, and
the amino acid of formulae (32) and (33) is optionally substituted, wherein the amino acid of formulae (32) and (33) is preferably substituted with R^{X11}—CO—NH— at the α-carbon atom which is covalently bound to the COOH-group in each one of formulae (32) and (33), wherein R^{X11} is selected from the group consisting of (C₁-C₁₀)alkyl, (C₅-C₁₀)aryl, and (C₁-C₅)alkyl-(C₅-C₁₀)aryl, R^{X11} being preferably methyl, and
wherein X42 is more preferably a residue of an amino acid selected from the group consisting of 1,13-diamino-4,7,10-trioxatridecan-succinamic acid [Ttds], 8-amino-3,6-dioxaoctanoic acid [O2Oc], 2-(4-(amino)piperidin-1-yl)acetic acid [APac], 4-carboxymethyl piperazine [PPac], 4-trans-aminomethylcyclohexane carboxylic acid [4Amc], β-alanine [Bal], γ-aminobutyric acid [Gab], 5-amino pentanoic acid [Ava], 6-aminohexanoic acid [Ahx], 3-aminomethyl-benzoic acid [Mamb], 4-aminomethyl-benzoic acid [Pamb] and an amino acid of formula (35):

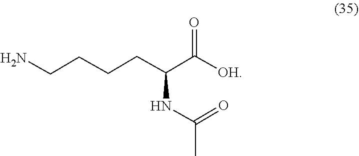

106-109. (canceled)

110. The compound of claim 91, wherein the effector E4 is selected from the group consisting of:
(α) a moiety derived from a chromophore, wherein the chromophore is preferably selected from (α1) a phosphorophore and (α2) a fluorophore such as fluorescein or rhodamine; and
(β) a chelator optionally comprising a chelated nuclide; and
(γ) a moiety derived from a drug, preferably from a cytotoxic drug.

111. The compound of claim 1, wherein the cyclic peptide is a cyclic peptide of formula (1g): 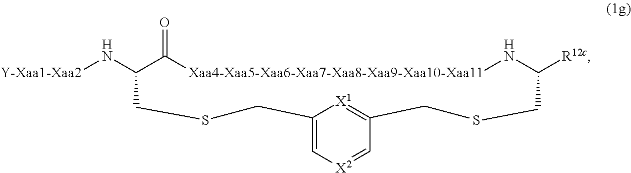
wherein R^{12c} is preferably selected from:
the group consisting of —CONH₂ and —COOH, or
the group consisting of —CO—Z6 and —CH₂—Z6, and Z6 comprises an effector E6 and a linker moiety L6,
wherein the linker moiety L6 preferably covalently links the effector E6 to a carbon atom of R^{12c},
and wherein R^{12c} is more preferably —CO—Z6 and the linker moiety L6 provides (a) a first amino group forming a covalent bond to carbonyl carbon atom of R^{12c}, and (b) a second amino group forming a covalent bond to the effector.

112-115. (canceled)

116. The compound of claim 111, wherein the linker moiety L6 is either X61 or a residue selected from the group consisting of X61-X62 and X62-X61, wherein:
X61 is a residue of a diamine providing a first amino group and a second amino group,
X62 is a residue of an amino acid providing an amino group and a carboxy group,
X61-X62 is a residue of a diamine, wherein the diamine provides a first amino group and a second amino group,
wherein the first amino group is the first amino group of X61,
the second amino group is the amino group of X62, and
the second amino group of X61 forms an amide bond with the carboxy group of X62, and
X62-X61 is a residue of a diamine, wherein the diamine provides a first amino group and a second amino group,
wherein the first amino group is the amino group of X62,
the second amino group is the second amino group of X61, and
the carboxy group of X62 forms an amide bond with the first amino group of X61,
wherein X61 is preferably a residue of a diamine which is selected from the group consisting of a diamine of any one of formulae (35-37): 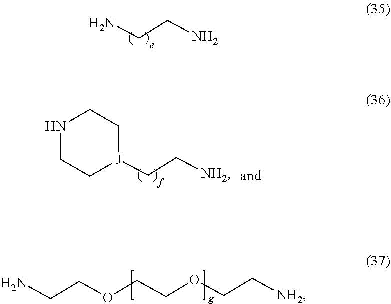
wherein
e is 1, 2, 3, 4, 5, 6, 7, 8, 9, or 10,
f is 0, 1, 2, 3, 4, 5 or 6,
g is 0, 1, 2, 3, 4, 5, 6, 7, 8, 9, 10, 11, or 12,
the diamine of any one of formulae (35) and (36) is optionally substituted with —CONH₂, and wherein J is selected from the group consisting of CH and N, and
wherein, in the diamine of any one of formulae (35) and (36), the carbon atom which is substituted with a nitrogen atom is preferably further substituted with —CONH₂.

117. (canceled)

118. (canceled)

119. The compound of claim 116, wherein X61 is a residue of a diamine selected from the group consisting of 1,3-diaminopropane [Apr], 1,5-diaminopentane [Ape], diaminobutane, ethylendiamine, a diamine of formula (39), and a diamine of formula (40): 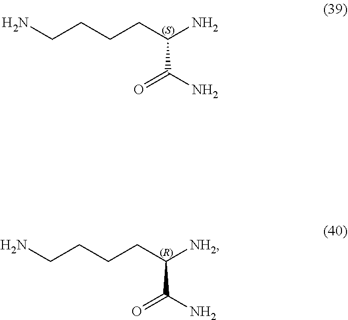
and/or
wherein X62 is a residue of an amino acid selected from the group consisting of 1,13-diamino-4,7,10-trioxatridecan-succinamic acid [Ttds], 8-amino-3,6-dioxaoctanoic acid [O2Oc], 2-(4-(amino)piperidin-1-yl)acetic acid [APac], 4-carboxymethyl piperazine [PPac], 4-trans-aminomethylcyclohexane carboxylic acid [4Amc] and an amino acid according to any one of formulae (32)-(33): 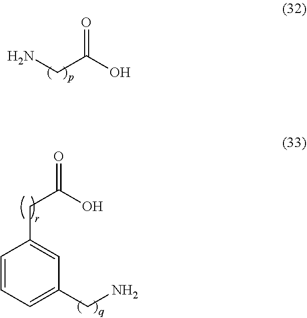
and the ortho- and para-substituted isomers thereof, and 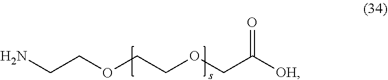
wherein
p is 2, 3, 4, 5, 6, 7, 8, 9, or 10,
g is 0, 1, 2, 3, or 4,
r is 0, 1, 2, 3, or 4,
s is 0, 1, 2, 3, 4, 5, 6, 7, 8, 9, 10, 11 or 12,
the amino acid of formula (32) and of formula (33) is each optionally substituted,
wherein the amino acid of formula (32) and of formula (33) is preferably each substituted with R^{X11}—CO—NH— at the α-carbon atom which is covalently bound to the COOH-group in formulae (32) and (33), wherein R^{X11} is (C₁-C₁₀)alkyl, (C₅-C₁₀)aryl, and (C₁-C₅)alkyl-(C₅-C₁₀)aryl, R^{X11} being preferably methyl,
wherein X62 is more preferably a residue of an amino acid selected from the group consisting of 1,13-diamino-4,7,10-trioxatridecan-succinamic acid [Ttds], 8-amino-3,6-dioxaoctanoic acid [O2Oc], 2-(4-(amino)piperidin-1-yl)acetic acid [APac], 4-Carboxymethyl piperazine [PPac], 4-trans-aminomethylcyclohexane carboxylic acid [4Amc], β-alanine [Bal], γ-aminobutyric acid [Gab], 5-amino pentanoic acid [Ava], 6-aminohexanoic acid [Ahx], 3-aminomethyl-benzoic acid [Mamb], 4-aminomethyl-benzoic acid [Pamb] and an amino acid of formula (35): 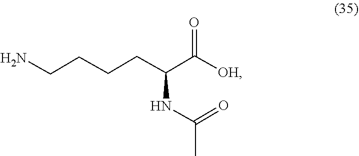
and
wherein X62 is most preferably a residue of an amino acid selected from the group consisting of 1,13-diamino-4,7,10-trioxatridecan-succinamic acid [Ttds], 8-amino-3,6-dioxaoctanoic acid [O2Oc], 3-aminomethyl-benzoic acid [Mamb], 4-aminomethyl-benzoic acid [Pamb].

120-124. (canceled)

125. The compound of claim 111, wherein the effector E6 is selected from the group consisting of:
(α) a moiety derived from a chromophore, wherein the chromophore is preferably selected from (α1) a phosphorophore and (α2) a fluorophore such as fluorescein or rhodamine; and
(β) a chelator optionally comprising a chelated nuclide; and
(γ) a moiety derived from a drug, preferably from a cytotoxic drug.

126. The compound of claim 1, wherein the compound is selected from the group consisting of:
compound DOTA-PPAc-Gln-[Cys(3MeBn)-Glu-pro-Asp-Af3(Cpsu)-Leu-Thr-Trp-Ser-Cys]-NH₂ (3BP-4452) of the following formula: 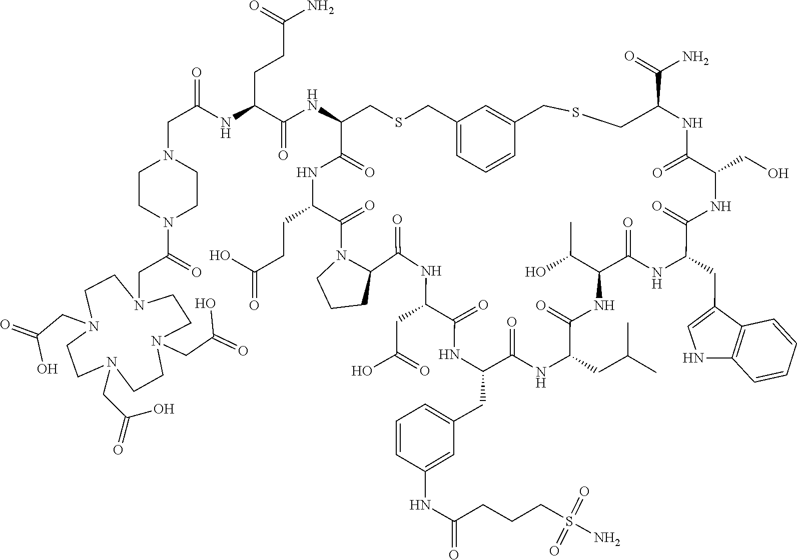 [SMILES: CC(C)C[C@@H]1NC(=O)[C@H](Cc2cccc(NC(=O)CCCS(N)(=O)=O)c2)NC(=O)[C@H](CC(=O)O)NC(=O)[C@H]2CCCN2C(=O)[C@H](CCC(=O)O)NC(=O)[C@@H](NC(=O)[C@H](CCC(N)=O)NC(=O)CN2CCN(C(=O)CN3CCN(CC(=O)O)CCN(CC(=O)O)CCN(CC(=O)O)CC3)CC2)CSCc2cccc(c2)CSC[C@@H](C(N)=O)NC(=O)[C@H](CO)NC(=O)[C@H](Cc2c[nH]c3ccccc23)NC(=O)[C@H]([C@@H](C)O)NC1=O]
compound DOTA-Gln-[Cys(3MeBn)-Glu-pro-Asp-Aph(SaPr)-Leu-Thr-Trp-Ser-Cys]-NH₂ (3BP-4501) of the following formula: 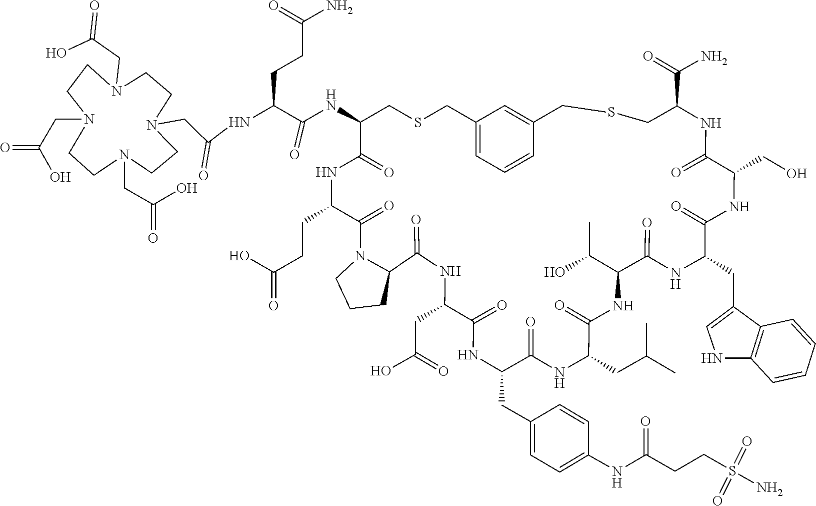 [SMILES: CC(C)C[C@@H]1NC(=O)[C@H](Cc2ccc(NC(=O)CCS(N)(=O)=O)cc2)NC(=O)[C@H](CC(=O)O)NC(=O)[C@H]2CCCN2C(=O)[C@H](CCC(=O)O)NC(=O)[C@@H](NC(=O)[C@H](CCC(N)=O)NC(=O)CN2CCN(CC(=O)O)CCN(CC(=O)O)CCN(CC(=O)O)CC2)CSCc2cccc(c2)CSC[C@@H](C(N)=O)NC(=O)[C@H](CO)NC(=O)[C@H](Cc2c[nH]c3ccccc23)NC(=O)[C@H]([C@@H](C)O)NC1=O]
compound DOTA-{Glu-[Cys(3MeBn)-Glu-pro-Asp-Af3(Cpsu)-Leu-Thr-Trp-Dap}-Cys]-NH₂ (3BP-4503) of the following formula: 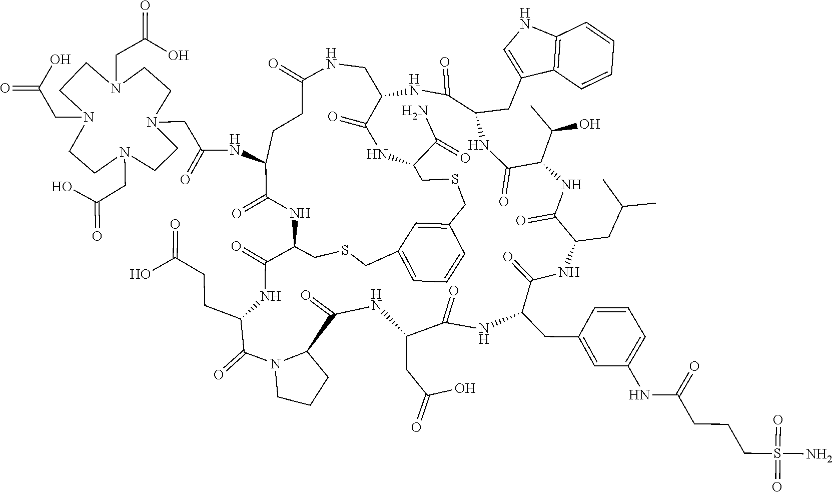 [SMILES: CC(C)C[C@@H]1NC(=O)[C@H](Cc2cccc(NC(=O)CCCS(N)(=O)=O)c2)NC(=O)[C@H](CC(=O)O)NC(=O)[C@H]2CCCN2C(=O)[C@H](CCC(=O)O)NC(=O)[C@@H]2CSCc3cccc(c3)CSC[C@@H](C(N)=O)NC(=O)[C@H](CNC(=O)CC[C@H](NC(=O)CN3CCN(CC(=O)O)CCN(CC(=O)O)CCN(CC(=O)O)CC3)C(=O)N2)NC(=O)[C@H](Cc2c[nH]c3ccccc23)NC(=O)[C@H]([C@@H](C)O)NC1=O]
compound Ac-Val-Tyr-[Cys(3MeBn)-Glu-pro-Asp-Trp-Leu-Thr-Trp-Glu(NH-Apr-DOTA)-Cys]-NH₂ (3BP-3478) of the following formula: 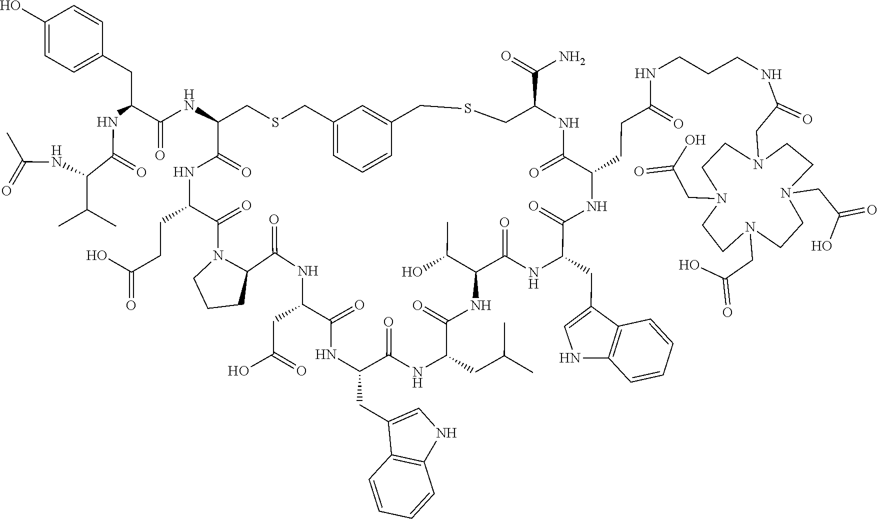 [SMILES: CC(=O)N[C@H](C(=O)N[C@@H](Cc1ccc(O)cc1)C(=O)N[C@H]1CSCc2cccc(c2)CSC[C@@H](C(N)=O)NC(=O)[C@H](CCC(=O)NCCCNC(=O)CN2CCN(CC(=O)O)CCN(CC(=O)O)CCN(CC(=O)O)CC2)NC(=O)[C@H](Cc2c[nH]c3ccccc23)NC(=O)[C@H]([C@@H](C)O)NC(=O)[C@H](CC(C)C)NC(=O)[C@H](Cc2c[nH]c3ccccc23)NC(=O)[C@H](CC(=O)O)NC(=O)[C@H]2CCCN2C(=O)[C@H](CCC(=O)O)NC1=O)C(C)C]
compound DOTA-APAc-Val-Tyr-[Cys(3MeBn)-Glu-pro-Asp-Trp-Leu-Thr-Trp-Ser-Cys]-NH₂ (3BP-3583) of the following formula: 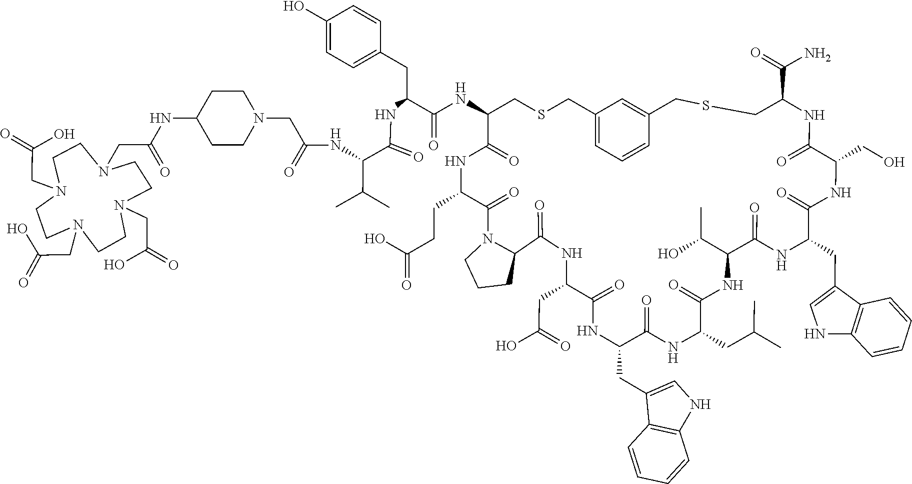 [SMILES: CC(C)C[C@@H]1NC(=O)[C@H](Cc2c[nH]c3ccccc23)NC(=O)[C@H](CC(=O)O)NC(=O)[C@H]2CCCN2C(=O)[C@H](CCC(=O)O)NC(=O)[C@@H](NC(=O)[C@H](Cc2ccc(O)cc2)NC(=O)[C@@H](NC(=O)CN2CCC(NC(=O)CN3CCN(CC(=O)O)CCN(CC(=O)O)CCN(CC(=O)O)CC3)CC2)C(C)C)CSCc2cccc(c2)CSC[C@@H](C(N)=O)NC(=O)[C@H](CO)NC(=O)[C@H](Cc2c[nH]c3ccccc23)NC(=O)[C@H]([C@@H](C)O)NC1=O]
compound Ac-Lys(DOTA)-Val-Tyr-[Cys(3MeBn)-Glu-pro-Asp-Trp-Leu-Thr-Trp-Ser-Cys]-NH₂ (3BP-3840) of the following formula: 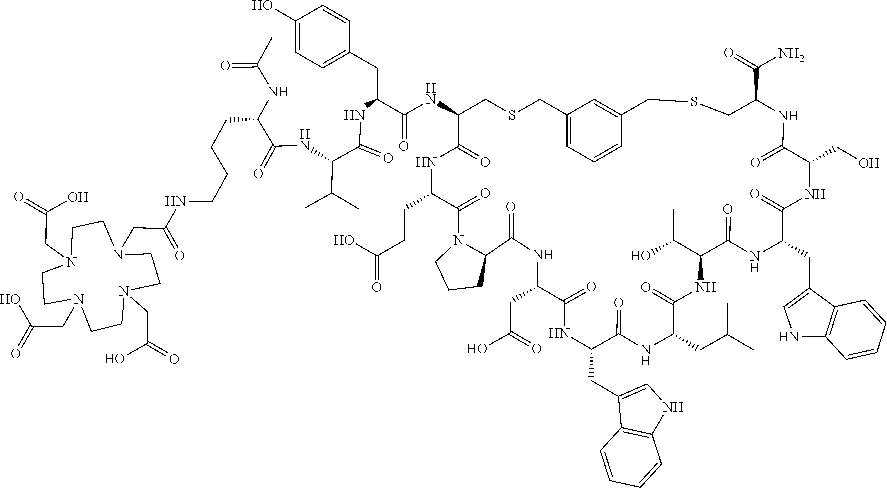 [SMILES: CC(=O)N[C@@H](CCCCNC(=O)CN1CCN(CC(=O)O)CCN(CC(=O)O)CCN(CC(=O)O)CC1)C(=O)N[C@H](C(=O)N[C@@H](Cc1ccc(O)cc1)C(=O)N[C@H]1CSCc2cccc(c2)CSC[C@@H](C(N)=O)NC(=O)[C@H](CO)NC(=O)[C@H](Cc2c[nH]c3ccccc23)NC(=O)[C@H]([C@@H](C)O)NC(=O)[C@H](CC(C)C)NC(=O)[C@H](Cc2c[nH]c3ccccc23)NC(=O)[C@H](CC(=O)O)NC(=O)[C@H]2CCCN2C(=O)[C@H](CCC(=O)O)NC1=O)C(C)C]
compound DOTA-APAc-Val-{Glu-[Cys(3MeBn)-Glu-pro-Asp-Trp-Leu-Thr-Trp-Dap}-Cys]-NH₂ (3BP-4175) of the following formula: 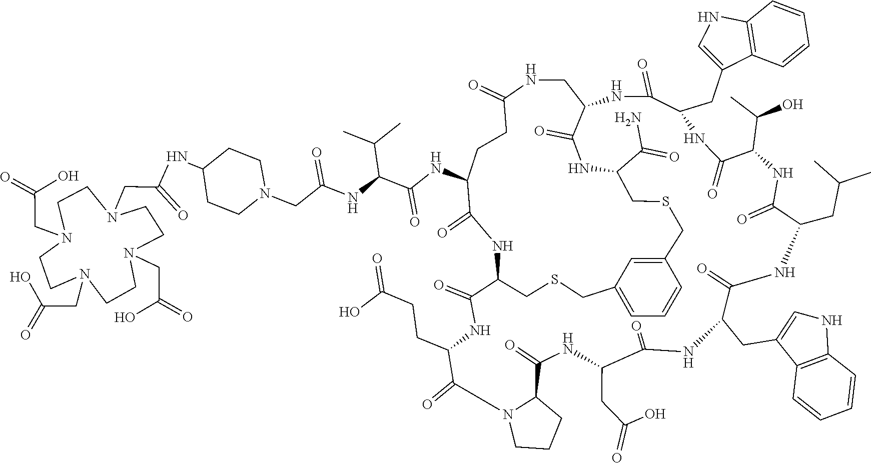 [SMILES: CC(C)C[C@@H]1NC(=O)[C@H](Cc2c[nH]c3ccccc23)NC(=O)[C@H](CC(=O)O)NC(=O)[C@H]2CCCN2C(=O)[C@H](CCC(=O)O)NC(=O)[C@@H]2CSCc3cccc(c3)CSC[C@@H](C(N)=O)NC(=O)[C@H](CNC(=O)CC[C@H](NC(=O)[C@@H](NC(=O)CN3CCC(NC(=O)CN4CCN(CC(=O)O)CCN(CC(=O)O)CCN(CC(=O)O)CC4)CC3)C(C)C)C(=O)N2)NC(=O)[C@H](Cc2c[nH]c3ccccc23)NC(=O)[C@H]([C@@H](C)O)NC1=O]
compound DOTA-APAc-Val-Tyr-[Cys(3MeBn)-Glu-pro-Asp-Af3(HO-Succinyl)-Leu-Thr-Trp-Ser-Cys]-NH₂ (3BP-4237) of the following formula: 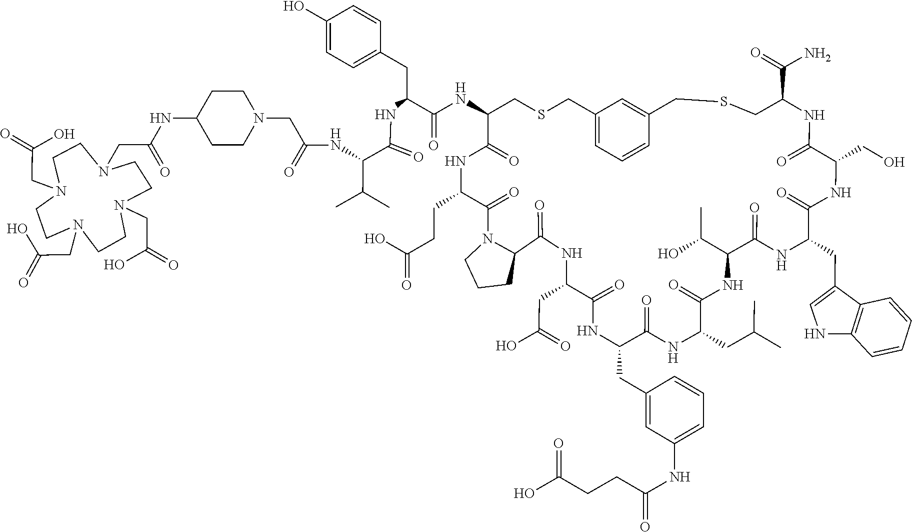 [SMILES: CC(C)C[C@@H]1NC(=O)[C@H](Cc2cccc(NC(=O)CCC(=O)O)c2)NC(=O)[C@H](CC(=O)O)NC(=O)[C@H]2CCCN2C(=O)[C@H](CCC(=O)O)NC(=O)[C@@H](NC(=O)[C@H](Cc2ccc(O)cc2)NC(=O)[C@@H](NC(=O)CN2CCC(NC(=O)CN3CCN(CC(=O)O)CCN(CC(=O)O)CCN(CC(=O)O)CC3)CC2)C(C)C)CSCc2cccc(c2)CSC[C@@H](C(N)=O)NC(=O)[C@H](CO)NC(=O)[C@H](Cc2c[nH]c3ccccc23)NC(=O)[C@H]([C@@H](C)O)NC1=O]
compound DOTA-APAc-Val-Tyr-[Cys(3MeBn)-Glu-pro-Asp-Af3(Cpsu)-Leu-Thr-Trp-Ser-Cys]-NH₂ (3BP-4369) of the following formula: 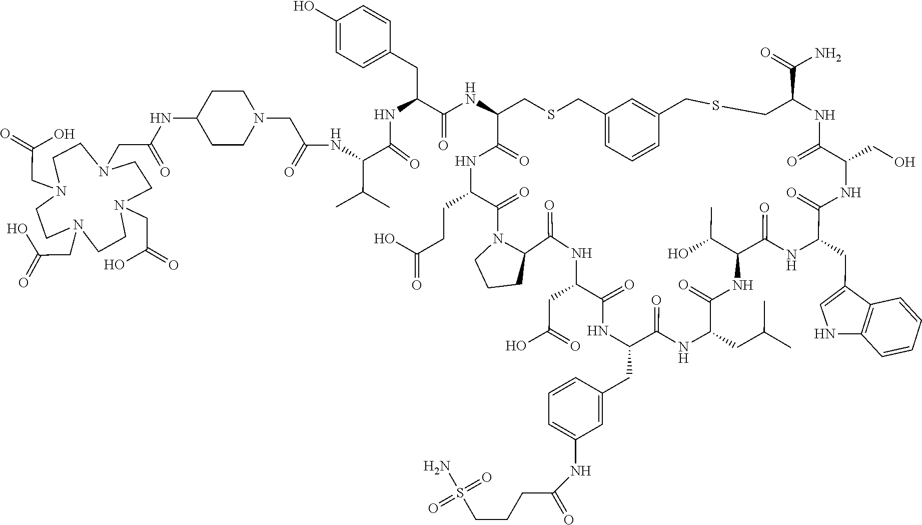 [SMILES: CC(C)C[C@@H]1NC(=O)[C@H](Cc2cccc(NC(=O)CCCS(N)(=O)=O)c2)NC(=O)[C@H](CC(=O)O)NC(=O)[C@H]2CCCN2C(=O)[C@H](CCC(=O)O)NC(=O)[C@@H](NC(=O)[C@H](Cc2ccc(O)cc2)NC(=O)[C@@H](NC(=O)CN2CCC(NC(=O)CN3CCN(CC(=O)O)CCN(CC(=O)O)CCN(CC(=O)O)CC3)CC2)C(C)C)CSCc2cccc(c2)CSC[C@@H](C(N)=O)NC(=O)[C@H](CO)NC(=O)[C@H](Cc2c[nH]c3ccccc23)NC(=O)[C@H]([C@@H](C)O)NC1=O]
compound DOTA-APAc-Val-Tyr-[Cys(3MeBn)-Glu-pro-Asp-Aph(SaPr)-Leu-Thr-Trp-Ser-Cys]-NH₂ (3BP-4400) of the following formula: 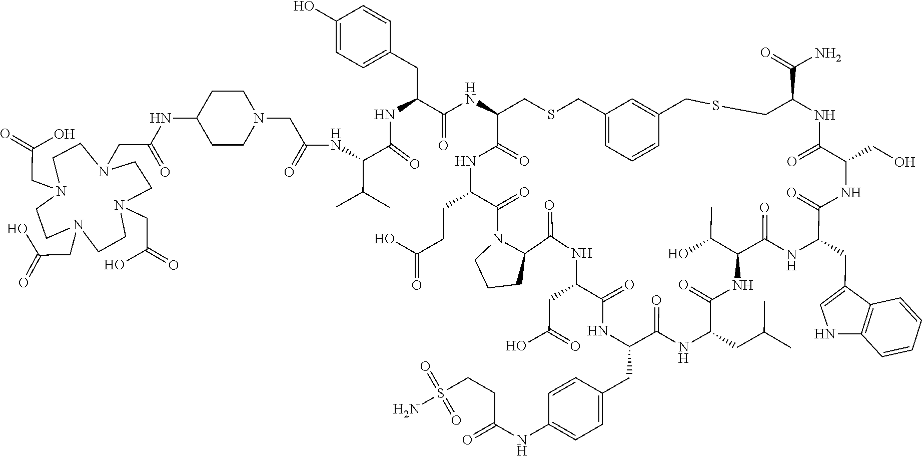 [SMILES: CC(C)C[C@@H]1NC(=O)[C@H](Cc2ccc(NC(=O)CCS(N)(=O)=O)cc2)NC(=O)[C@H](CC(=O)O)NC(=O)[C@H]2CCCN2C(=O)[C@H](CCC(=O)O)NC(=O)[C@@H](NC(=O)[C@H](Cc2ccc(O)cc2)NC(=O)[C@@H](NC(=O)CN2CCC(NC(=O)CN3CCN(CC(=O)O)CCN(CC(=O)O)CCN(CC(=O)O)CC3)CC2)C(C)C)CSCc2cccc(c2)CSC[C@@H](C(N)=O)NC(=O)[C@H](CO)NC(=O)[C@H](Cc2c[nH]c3ccccc23)NC(=O)[C@H]([C@@H](C)O)NC1=O]
compound DOTA-PPAc-Tyr-[Cys(3MeBn)-Glu-pro-Asp-Af3(Cpsu)-Leu-Thr-Trp-Ser-Cys]-NH₂ (3BP-4448) of the following formula: 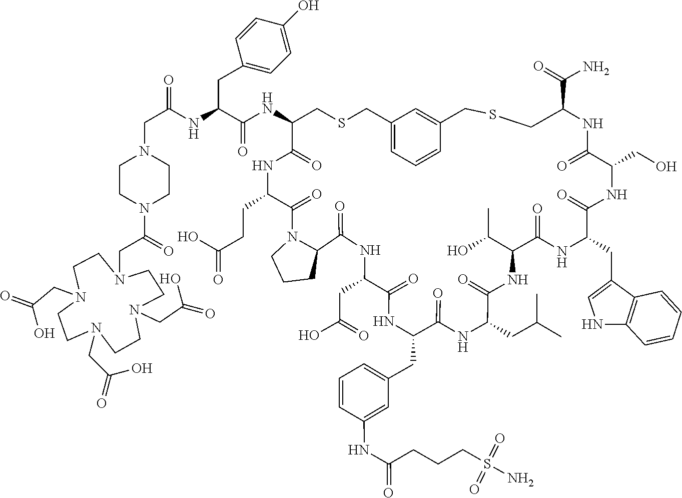 [SMILES: CC(C)C[C@@H]1NC(=O)[C@H](Cc2cccc(NC(=O)CCCS(N)(=O)=O)c2)NC(=O)[C@H](CC(=O)O)NC(=O)[C@H]2CCCN2C(=O)[C@H](CCC(=O)O)NC(=O)[C@@H](NC(=O)[C@H](Cc2ccc(O)cc2)NC(=O)CN2CCN(C(=O)CN3CCN(CC(=O)O)CCN(CC(=O)O)CCN(CC(=O)O)CC3)CC2)CSCc2cccc(c2)CSC[C@@H](C(N)=O)NC(=O)[C@H](CO)NC(=O)[C@H](Cc2c[nH]c3ccccc23)NC(=O)[C@H]([C@@H](C)O)NC1=O]
compound DOTA-Gln-[Cys(3MeBn)-Glu-pro-Asp-Af3(Cpsu)-Leu-Thr-Trp-Ser-Cys]-NH₂ (3BP-4453) of the following formula: 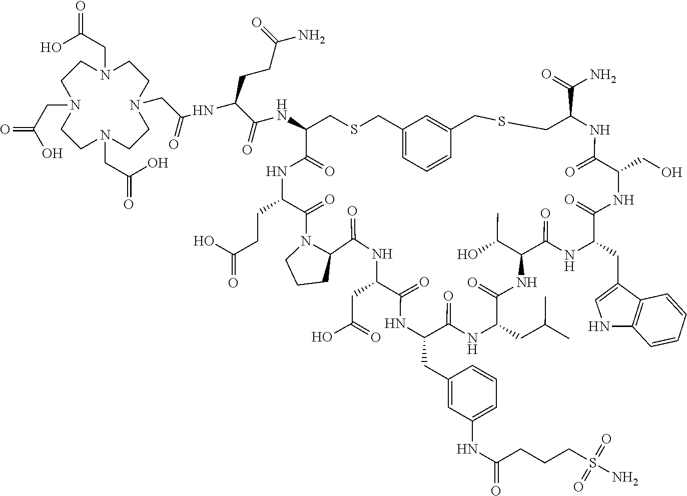 [SMILES: CC(C)C[C@@H]1NC(=O)[C@H](Cc2cccc(NC(=O)CCCS(N)(=O)=O)c2)NC(=O)[C@H](CC(=O)O)NC(=O)[C@H]2CCCN2C(=O)[C@H](CCC(=O)O)NC(=O)[C@@H](NC(=O)[C@H](CCC(N)=O)NC(=O)CN2CCN(CC(=O)O)CCN(CC(=O)O)CCN(CC(=O)O)CC2)CSCc2cccc(c2)CSC[C@@H](C(N)=O)NC(=O)[C@H](CO)NC(=O)[C@H](Cc2c[nH]c3ccccc23)NC(=O)[C@H]([C@@H](C)O)NC1=O]
compound DOTA-Rni-Tyr-[Cys(3MeBn)-Glu-pro-Asp-Af3(Cpsu)-Leu-Thr-Trp-Ser-Cys]-NH₂ (3BP-4455) of the following formula: 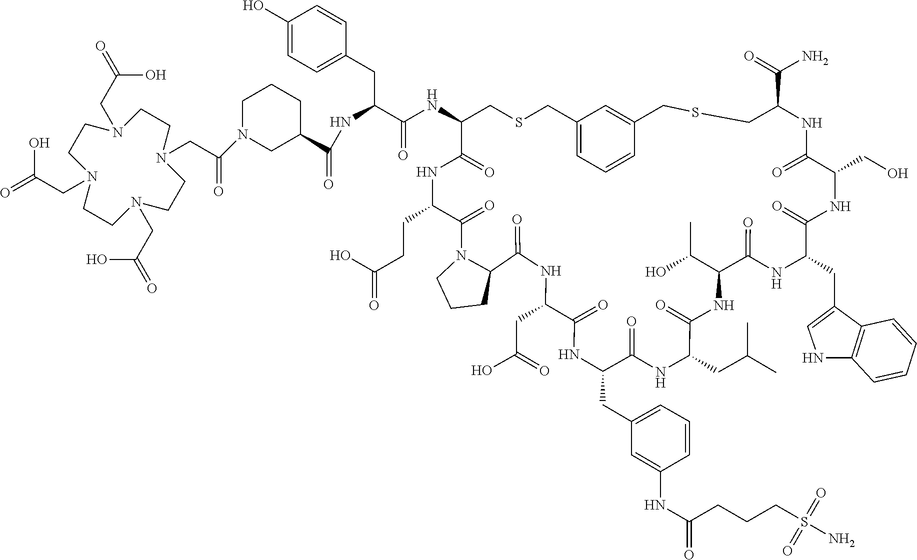 [SMILES: CC(C)C[C@@H]1NC(=O)[C@H](Cc2cccc(NC(=O)CCCS(N)(=O)=O)c2)NC(=O)[C@H](CC(=O)O)NC(=O)[C@H]2CCCN2C(=O)[C@H](CCC(=O)O)NC(=O)[C@@H](NC(=O)[C@H](Cc2ccc(O)cc2)NC(=O)[C@@H]2CCCN(C(=O)CN3CCN(CC(=O)O)CCN(CC(=O)O)CCN(CC(=O)O)CC3)C2)CSCc2cccc(c2)CSC[C@@H](C(N)=O)NC(=O)[C@H](CO)NC(=O)[C@H](Cc2c[nH]c3ccccc23)NC(=O)[C@H]([C@@H](C)O)NC1=O]
compound DOTA-PPAc-{Glu-[Cys(3MeBn)-Glu-pro-Asp-Aph(SaPr)-Leu-Thr-Trp-Dap}-Cys]-NH₂ (3BP-4504) of the following formula: 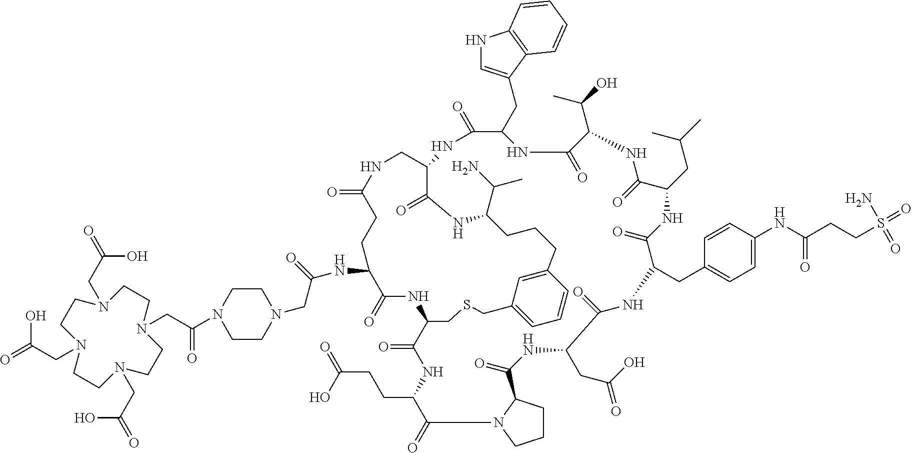
compound DOTA-{Glu-[Cys(3MeBn)-Glu-pro-Asp-Aph(SaPr)-Leu-Thr-Trp-Dap}-Cys]-NH₂ (3BP-4505) of the following formula: 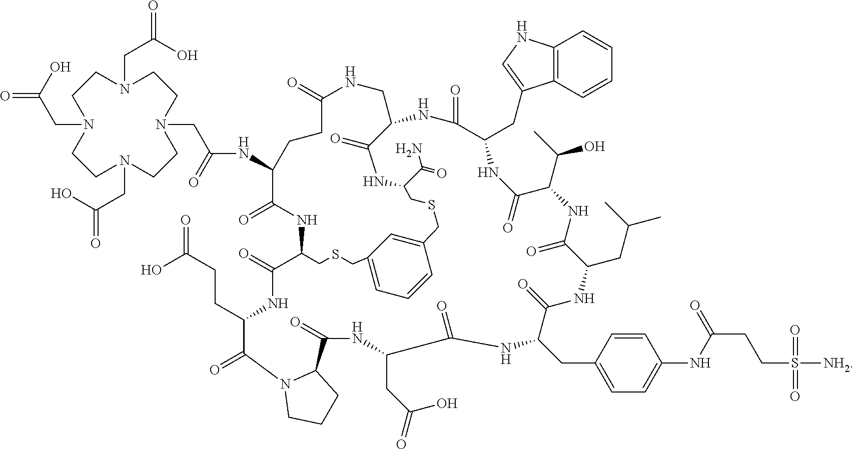 [SMILES: CC(C)C[C@@H]1NC(=O)[C@H](Cc2ccc(NC(=O)CCS(N)(=O)=O)cc2)NC(=O)[C@H](CC(=O)O)NC(=O)[C@H]2CCCN2C(=O)[C@H](CCC(=O)O)NC(=O)[C@@H]2CSCc3cccc(c3)CSC[C@@H](C(N)=O)NC(=O)[C@H](CNC(=O)CC[C@H](NC(=O)CN3CCN(CC(=O)O)CCN(CC(=O)O)CCN(CC(=O)O)CC3)C(=O)N2)NC(=O)[C@H](Cc2c[nH]c3ccccc23)NC(=O)[C@H]([C@@H](C)O)NC1=O]
wherein the compound is preferably selected from the group consisting of:
compound DOTA-PPAc-Gln-[Cys(3MeBn)-Glu-pro-Asp-Af3(Cpsu)-Leu-Thr-Trp-Ser-Cys]-NH₂ (3BP-4452) of the following formula: 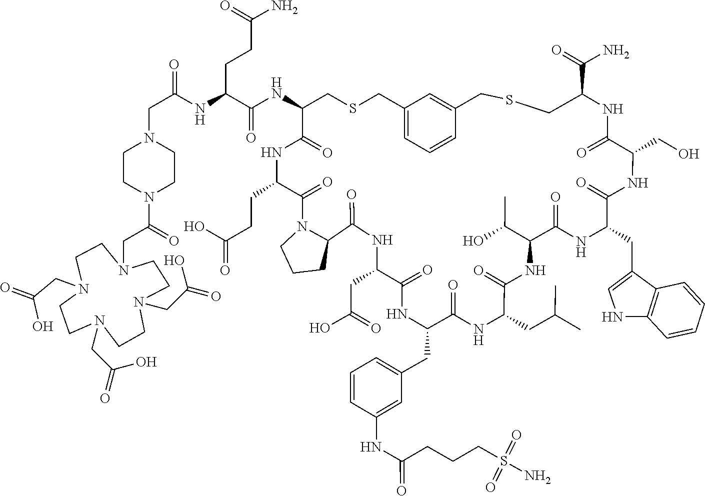 [SMILES: CC(C)C[C@@H]1NC(=O)[C@H](Cc2cccc(NC(=O)CCCS(N)(=O)=O)c2)NC(=O)[C@H](CC(=O)O)NC(=O)[C@H]2CCCN2C(=O)[C@H](CCC(=O)O)NC(=O)[C@@H](NC(=O)[C@H](CCC(N)=O)NC(=O)CN2CCN(C(=O)CN3CCN(CC(=O)O)CCN(CC(=O)O)CCN(CC(=O)O)CC3)CC2)CSCc2cccc(c2)CSC[C@@H](C(N)=O)NC(=O)[C@H](CO)NC(=O)[C@H](Cc2c[nH]c3ccccc23)NC(=O)[C@H]([C@@H](C)O)NC1=O]
compound DOTA-Gln-[Cys(3MeBn)-Glu-pro-Asp-Aph(SaPr)-Leu-Thr-Trp-Ser-Cys]-NH₂ (3BP-4501) of the following formula: 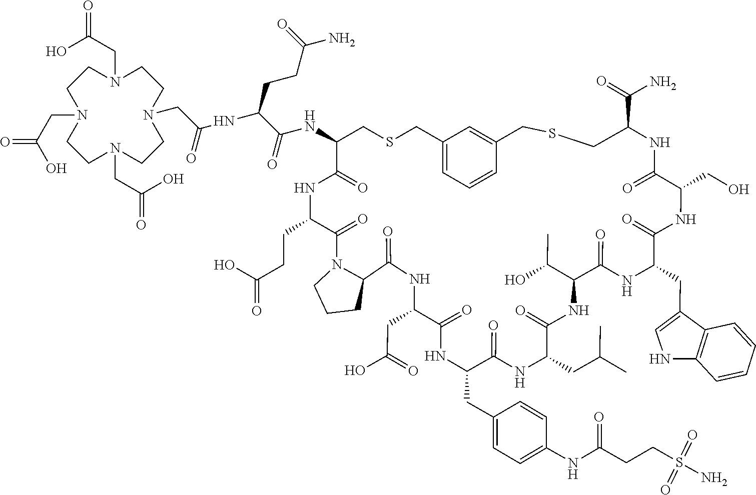 [SMILES: CC(C)C[C@@H]1NC(=O)[C@H](Cc2ccc(NC(=O)CCS(N)(=O)=O)cc2)NC(=O)[C@H](CC(=O)O)NC(=O)[C@H]2CCCN2C(=O)[C@H](CCC(=O)O)NC(=O)[C@@H](NC(=O)[C@H](CCC(N)=O)NC(=O)CN2CCN(CC(=O)O)CCN(CC(=O)O)CCN(CC(=O)O)CC2)CSCc2cccc(c2)CSC[C@@H](C(N)=O)NC(=O)[C@H](CO)NC(=O)[C@H](Cc2c[nH]c3ccccc23)NC(=O)[C@H]([C@@H](C)O)NC1=O]
compound DOTA-{Glu-[Cys (3MeBn)-Glu-pro-Asp-Af3(Cpsu)-Leu-Thr-Trp-Dap}-Cys]-NH₂ (3BP-4503) of the following formula: 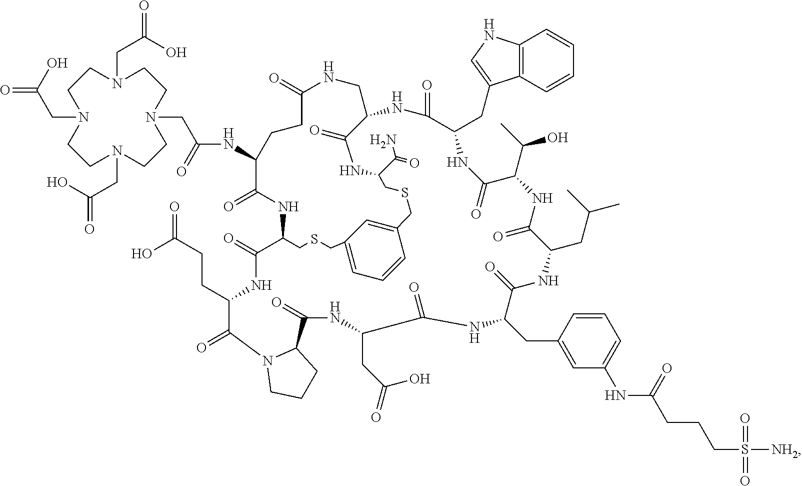 [SMILES: CC(C)C[C@@H]1NC(=O)[C@H](Cc2cccc(NC(=O)CCCS(N)(=O)=O)c2)NC(=O)[C@H](CC(=O)O)NC(=O)[C@H]2CCCN2C(=O)[C@H](CCC(=O)O)NC(=O)[C@@H]2CSCc3cccc(c3)CSC[C@@H](C(N)=O)NC(=O)[C@H](CNC(=O)CC[C@H](NC(=O)CN3CCN(CC(=O)O)CCN(CC(=O)O)CCN(CC(=O)O)CC3)C(=O)N2)NC(=O)[C@H](Cc2c[nH]c3ccccc23)NC(=O)[C@H]([C@@H](C)O)NC1=O]
wherein the compound is more preferably:
compound DOTA-PPAc-Gln-[Cys(3MeBn)-Glu-pro-Asp-Af3(Cpsu)-Leu-Thr-Trp-Ser-Cys]-NH₂ (3BP-4452) of the following formula: 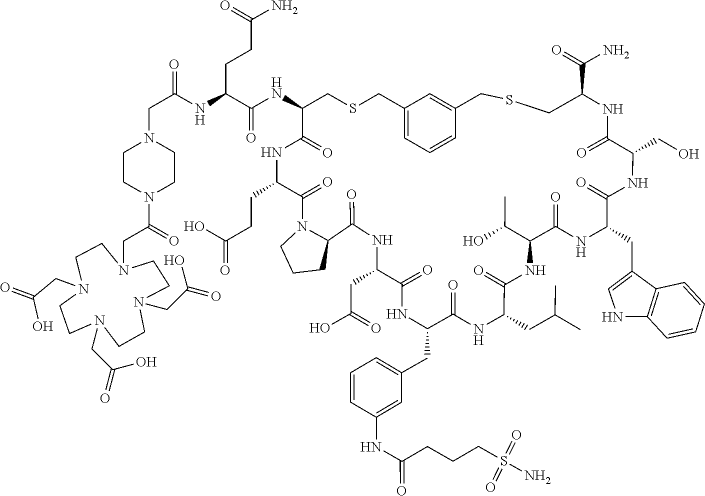
wherein, in the above compounds, DOTA may be replaced by a chelator selected from the group consisting of DOTAGA, DOTAM, DOTP, NOTA, NODAGA, NODA-MPAA, HBED, TETA, CB-TE2A, DTPA, CHX-A″-DTPA, DFO, Macropa, HOPO, TRAP, THP, DATA, NOPO, NOTP, PCTA, sarcophagine, FSC, NETA, NE3TA, H4octapa, pycup, HYNIC, NxS4-x (N₄, N2S2, N3S), ^{99m}Tc(CO)₃-chelators and their analogs, preferably from the group consisting of DOTAGA, DOTAM, NOTA, NODAGA, NODA-MPAA, NOPO, HBED, DTPA, CHX-A″-DTPA, CB-TE2A, Macropa, PCTA, N4, and analogs thereof, and more preferably from the group consisting of DOTAGA, NODAGA, and macropa, and their analogs thereof.

127-129. (canceled)

130. The compound of claim 1, wherein each effector E1, E3, E4 and E6, if present, is independently a chelator optionally comprising a chelated nuclide, the chelator being preferably selected from the group comprising DOTA, DOTAGA, DOTAM, DOTP, NOTA, NODAGA, NODA-MPAA, HBED, TETA, CB-TE2A, DTPA, CHX-A″-DTPA, DFO, Macropa, HOPO, TRAP, THP, DATA, NOPO, NOTP, PCTA, sarcophagine, FSC, NETA, NE3TA, H4octapa, pycup, HYNIC, NxS4-x (N4, N2S2, N3S), ^{99m}Tc(CO)₃-chelators and their analogs-, wherein the chelator is preferably selected from the group comprising DOTA, DOTAGA, DOTAM, NOTA, NODAGA, NODA-MPAA, NOPO, HBED, DTPA, CHX-A″-DTPA, CB-TE2A, Macropa, PCTA, N4, and analogs thereof, wherein the chelator is more preferably selected from the group comprising DOTA, DOTAGA, NODAGA, and macropa, and their analogs thereof.

131. (canceled)

132. (canceled)

133. The compound of claim 126, wherein the chelator comprises a chelated nuclide.

134. The compound of claim 133, wherein the chelated nuclide is a diagnostically active nuclide-, wherein the diagnostically active nuclide is preferably a diagnostically active radionuclide, wherein the nuclide is preferably selected from the group comprising ⁴³Sc, ⁴⁴Sc ⁵¹Mn, ⁵²Mn, ⁶⁴Cu, ⁶⁷Ga ⁶⁸Ga, ⁸⁶Y ⁸⁹Zr, ^{94m}Tc, ^{99m}Tc ¹¹¹In, ¹⁵²Tb ¹⁵⁵Tb, ¹⁷⁷Lu, ²⁰¹Tl, ²⁰³Pb, ¹⁸F, ⁷⁶Br, ⁷⁷Br, ¹²³I, ¹²⁴I, and ¹²⁵I, wherein the nuclide is more preferably selected form the group comprising ⁴³Sc, ⁴⁴Sc, ⁶⁴Cu, ⁶⁷Ga, ⁶⁸Ga, ⁸⁶Y ⁸⁹Zr, ¹¹¹In, ¹⁵²Tb ¹⁵⁵Tb, and ²⁰³Pb, and wherein the nuclide is most preferably selected from the group comprising ⁶⁴Cu, ⁶⁸Ga, ¹¹¹In, and ²⁰³Pb.

135-138. (canceled)

139. The compound of claim 133, wherein the chelated nuclide is a therapeutically active nuclide-, wherein the therapeutically active nuclide is preferably a therapeutically active radionuclide, wherein the nuclide is preferably selected from the group comprising ⁴⁷Sc, ⁶⁷Cu, ⁸⁹Sr, ⁹⁰Y, ¹¹¹In, ¹⁵³Sm, ¹⁴⁹Tb, ¹⁶¹Tb, ¹⁷⁷Lu, ¹⁸⁶Re, ¹⁸⁸Re, ²¹²Pb, ²¹³Bi, ²²³Ra, ²²⁵Ac, ²²⁶Th, ²²⁷Th, ¹³¹I, and ²¹¹At, wherein the nuclide is more preferably selected from the group comprising ⁴⁷Sc, ⁶⁷Cu, ⁹⁰Y ¹⁶¹Tb, ¹⁷⁷Lu, ²¹²Pb, ²¹³Bi, ²²⁵Ac, and ²²⁷Th, wherein the nuclide is most preferably selected from the group comprising ⁹⁰Y, ¹⁶¹Tb, ¹⁷⁷Lu, ²¹²Pb, ²²⁵Ac, and ²²⁷Th.

140-143. (canceled)

144. The compound of claim 126, wherein the chelator comprises a chelated diagnostically active nuclide selected from ¹¹¹In, and ⁶⁸Ga.

145. The compound of claim 126, wherein the chelator comprises a chelated therapeutically active nuclide selected from ¹⁶¹Tb, ¹⁷⁷Lu, ²¹²Pb, and ²²⁵Ac.

146. (canceled)

147. (canceled)

148. A method of diagnosing a disease, wherein the compound of claim 144 is administered to a patient.

149. A method for the treatment of a disease wherein the compound of claim 145 is administered to a patient.

150. A method selected from:
a method for the identification of a subject, wherein the subject is likely to respond or likely not to respond to a treatment of a disease, wherein the method for the identification of a subject comprises carrying out a method of diagnosing a disease in which the compound of claim 144 is administered to a patient,
a method for the selection of a subject from a group of subjects, wherein the subject is likely to respond or likely not to respond to a treatment of a disease, wherein the method for the selection of a subject from a group of subjects comprises carrying out a method of diagnosing a disease in which the compound of claim 144 is administered to a patient,
a method for the stratification of a group of subjects into subjects which are likely to respond to a treatment of a disease, and into subjects which are not likely to respond to a treatment of a disease, wherein the method for the stratification of a group of subjects comprises carrying out a method of diagnosing a disease in which the compound of claim 144 is administered to a patient.

151. (canceled)

152. (canceled)

153. The compound for use of claim 150, wherein the disease is cancer-, wherein the cancer preferably is a solid cancer or a solid tumor, wherein the cancer is more preferably a hypoxic cancer, wherein the cancer is most preferably carbonic anhydrase IX expressing cancer.

154-156. (canceled)

157. The compound for use of claim 153, wherein the cancer is selected from the group consisting of clear cell renal cell carcinoma (ccRCC), colorectal carcinoma (CRC), pancreatic ductal adenocarcinoma (PDAC), glioblastoma (GBM), mesothelioma, cholangiocarcinoma (CCA), ovarian carcinoma, non-small cell lung cancer (NSCLC), brain cancer, pancreatic cancer, thyroid cancer, lung cancer, renal cancer, breast cancer, head and neck cancer, urothelial carcinoma and bladder cancer-, wherein the cancer is preferably selected from the group consisting of squamous non-small cell lung cancer (Sq. NSCLC), triple-negative breast cancer (TNBC), squamous cell carcinoma of head and neck (SCCHN), clear cell renal cell carcinoma (ccRCC), colorectal carcinoma (CRC), and pancreatic ductal adenocarcinoma (PDAC).

158. (canceled)

159. The compound for use of claim 153, wherein the cancer comprises CAIX expressing cancer-associated fibroblasts (CAFs).

160. The compound for claim 148, wherein the disease is a cancer associated with an alteration of the von Hippel-Lindau gene, wherein the cancer is preferably selected from the group consisting of clear cell renal cell carcinoma (ccRCC), renal cell carcinoma (RCC), lung cancer, colorectal carcinoma (CRC), and bladder cancer, wherein the cancer is more preferably clear cell renal cell carcinoma (ccRCC).

161. (canceled)

162. (canceled)

163. A compositing comprising a compound of claim 1 and a pharmaceutically acceptable excipient, wherein the composition is preferably a pharmaceutical composition.

164. (canceled)

165. A kit comprising a compound of claim 1 and one or more optional excipient(s) and optionally one or more device(s), wherein the device(s) is/are preferably selected from the group comprising a labeling device, a purification device, a handling device, a radioprotection device, an analytical device or an administration device.

166. (canceled)


SEQUENCE LISTING (9 sequences)

SEQ ID NO: 1 - protein, 12 aa, synthetic construct
  REGION 1..12: note = Synthetic
YNTNHVPLSPKY

SEQ ID NO: 2 - protein, 12 aa, synthetic construct
  REGION 1..12: note = Synthetic
NMPKDVTTRMSS

SEQ ID NO: 3 - protein, 7 aa, synthetic construct
  REGION 1..7: note = Synthetic
NHYPLSP

SEQ ID NO: 4 - protein, 459 aa, Homo sapiens
MAPLCPSPWLPLLIPAPAPGLTVQLLLSLLLLVPVHPQRLPRMQEDSPLGGGSSGEDDPLGEEDLPSEEDSPREEDPPGEEDLPGEEDLPGEEDLPEVKPKSEEEGSLKLEDLPTVEAPGDPQEPQNNAHRDKEGDDQSHWRYGGDPPWPRVSPACAGRFQSPVDIRPQLAAFCPALRPLELLGFQLPPLPELRLRNNGHSVQLTLPPGLEMALGPGREYRALQLHLHWGAAGRPGSEHTVEGHRFPAEIHVVHLSTAFARVDEALGRPGGLAVLAAFLEEGPEENSAYEQLLSRLEEIAEEGSETQVPGLDISALLPSDFSRYFQYEGSLTTPPCAQGVIWTVFNQTVMLSAKQLHTLSDTLWGPGDSRLQLNFRATQPLNGRVIEASFPAGVDSSPRAAEPVQLNSCLAAGDILALVFGLLFAVTSVAFLVQMRRQHRRGTKGGVSYRPAEVAETGA

SEQ ID NO: 5 - protein, 312 aa, Homo sapiens
MRMLLALLALSAARPSASAESHWCYEVQAESSNYPCLVPVKWGGNCQKDRQSPINIVTTKAKVDKKLGRFFFSGYDKKQTWTVQNNGHSVMMLLENKASISGGGLPAPYQAKQLHLHWSDLPYKGSEHSLDGEHFAMEMHIVHEKEKGTSRNVKEAQDPEDEIAVLAFLVEAGTQVNEGFQPLVEALSNIPKPEMSTTMAESSLLDLLPKEEKLRHYFRYLGSLTTPTCDEKVVWTVFREPIQLHREQILAFSQKLYYDKEQTVSMKDNVRPLQQLGQRTVIKSGAPGRPLPWALPALLGPMLACLLAGFLR

SEQ ID NO: 6 - protein, 354 aa, Homo sapiens
MPRRSLHAAAVLLLVILKEQPSSPAPVNGSKWTYFGPDGENSWSKKYPSCGGLLQSPIDLHSDILQYDASLTPLEFQGYNLSANKQFLLTNNGHSVKLNLPSDMHIQGLQSRYSATQLHLHWGNPNDPHGSEHTVSGQHFAAELHIVHYNSDLYPDASTASNKSEGLAVLAVLIEMGSFNPSYDKIFSHLQHVKYKGQEAFVPGFNIEELLPERTAEYYRYRGSLTTPPCNPTVLWTVFRNPVQISQEQLLALETALYCTHMDDPSPREMINNFRQVQKFDERLVYTSFSQVQVCTAAGLSLGIILSLALAGILGICIVVVVSIWLFRRKSIKKGDNKGVIYKPATKMETEAHA

SEQ ID NO: 7 - protein, 337 aa, Homo sapiens
MLFSALLLEVIWILAADGGQHWTYEGPHGQDHWPASYPECGNNAQSPIDIQTDSVTFDPDLPALQPHGYDQPGTEPLDLHNNGHTVQLSLPSTLYLGGLPRKYVAAQLHLHWGQKGSPGGSEHQINSEATFAELHIVHYDSDSYDSLSEAAERPQGLAVLGILIEVGETKNIAYEHILSHLHEVRHKDQKTSVPPFNLRELLPKQLGQYFRYNGSLTTPPCYQSVLWTVFYRRSQISMEQLEKLQGTLFSTEEEPSKLLVQNYRALQPLNQRMVFASFIQAGSSYTTGEMLSLGVGILVGCLCLLLAVYFIARKIRKKRLENRKSVVFTSAQATTEA

SEQ ID NO: 8 - protein, 461 aa, Canis lupus
MAPLCPSPWLPLLIPAPSLGPGVQLLLILPLLVPVHPQSLSRIQGTPGLGGDSSGEDDQLDEENLPSEEDPHGEEDPPGEEDPPGEEDPPGEEDPSGMKTEPGKEDSLKLEDLPTVEAPRDTPGSQNNAHRHNKGDDHSHWRYGGDPPWPQVSPACAGRFQSPVDIRPQLAALSPALPPLELLGFELPPLPELRLRNNGHTVQLTLPPGLEMALGPGREYRALQLHLHWGAAGRPGSEHTVDGHRFPAEIHVVHISTAFAKVEEALGRPGGLAVLAAFLQEGPEENSAYEQLLSHLEEITEEDSETWVPGLDVSALLPSDLSRYFRYEGSLTTPPCAQGVIWTVFNQTVRLSAKQLHTLSGSLWGPDDSRLQLNFRATQPLNGRIIEASFPAQVESSPRSFEPVHLNSCLGAGDILAVVFGLLFAVTSIAFIVQMRKQGRLRNGTKGNVSYHPAEVTETVA

SEQ ID NO: 9 - protein, 437 aa, Mus musculus
MASLGPSPWAPLSTPAPTAQLLLFLLLQVSAQPQGLSGMQGEPSLGDSSSGEDELGVDVLPSEEDAPEEADPPDGEDPPEVNSEDRMEESLGLEDLSTPEAPEHSQGSHGDEKGGGHSHWSYGGTLLWPQVSPACAGRFQSPVDIRLERTAFCRTLQPLELLGYELQPLPELSLSNNGHTVQLTLPPGLKMALGPGQEYRALQLHLHWGTSDHPGSEHTVNGHRFPAEIHVVHLSTAFSELHEALGRPGGLAVLAAFLQESPEENSAYEQLLSHLEEISEEGSKIEIPGLDVSALLPSDLSRYYRYEGSLTTPPCSQGVIWTVFNETVKLSAKQLHTLSVSLWGPRDSRLQLNFRATQPLNGRTIEASFPAAEDSSPEPVHVNSCFTAGDILALVFGLLFAVTSIAFLLQLRRQHRHRSGTKDRVSYSPAEMTETGA